SEQUENCE LISTING (7 sequences)

SEQ ID NO: 1 - DNA, 1450 bp, Homo sapiens
  CDS (273)..(1316)
gtgagcttgctccttaagttacaggaactctccttataatagacacttcattttcctagtccatccctcatgaaaaatgactgaccactgctgggcagcaggagggatgataatcctaactccaatcactggcaactcctgagatcagaggaaaaccagcaacagcgtgggagtttggggagaggcattccataccagattctgtggcctgcaggtgacatgctgcctaagagaagcaggagtctgtgacagccaccccaacacgtgacgtcatggccagtagggaagatgagctgaggaactgtgtggtatgtggggaccaagccacaggctaccactttaatgcgctgacttgtgagggctgcaagggtttcttcaggagaacagtcagcaaaagcattggtcccacctgcccctttgctggaagctgtgaagtcagcaagactcagaggcgccactgcccagcctgcaggttgcagaagtgcttagatgctggcatgaggaaagacatgatactgtcggcagaagccctggcattgcggcgagcaaagcaggcccagcggcgggcacagcaaacacctgtgcaactgagtaaggagcaagaagagctgatccggacactcctgggggcccacacccgccacatgggcaccatgtttgaacagtttgtgcagtttaggcctccagctcatctgttcatccatcaccagcccttgcccaccctggcccctgtgctgcctctggtcacacacttcgcagacatcaacactttcatggtactgcaagtcatcaagtttactaaggacctgcccgtcttccgttccctgcccattgaagaccagatctcccttctcaagggagcagctgtggaaatctgtcacatcgtactcaataccactttctgtctccaaacacaaaacttcctctgcgggcctcttcgctacacaattgaagatggagcccgtgtggggttccaggtagagtttttggagttgctctttcacttccatggaacactacgaaaactgcagctccaagagcctgagtatgtgctcttggctgccatggccctcttctctcctgaccgacctggagttacccagagagatgagattgatcagctgcaagaggagatggcactgactctgcaaagctacatcaagggccagcagcgaaggccccgggatcggtttctgtatgcgaagttgctaggcctgctggctgagctccggagcattaatgaggcctacgggtaccaaatccagcacatccagggcctgtctgccatgatgccgctgctccaggagatctgcagctgaggccatgctcacttccttccccagctcacctggaacaccctggatacactggagtgggaaaatgctgggaccaaagattgggccgggttcaaagggagcccagtggttgcaatgaaagactaaagcaaaac

SEQ ID NO: 2 - protein, 348 aa, Homo sapiens
MASREDELRNCVVCGDQATGYHFNALTCEGCKGFFRRTVSKSIGPTCPFAGSCEVSKTQRRHCPACRLQKCLDAGMRKDMILSAEALALRRAKQAQRRAQQTPVQLSKEQEELIRTLLGAHTRHMGTMFEQFVQFRPPAHLFIHHQPLPTLAPVLPLVTHFADINTFMVLQVIKFTKDLPVFRSLPIEDQISLLKGAAVEICHIVLNTTFCLQTQNFLCGPLRYTIEDGARVGFQVEFLELLFHFHGTLRKLQLQEPEYVLLAAMALFSPDRPGVTQRDEIDQLQEEMALTLQSYIKGQQRRPRDRFLYAKLLGLLAELRSINEAYGYQIQHIQGLSAMMPLLQEICS

SEQ ID NO: 3 - DNA, 714 bp, Homo sapiens
  CDS (1)..(714)
cctgtgcaactgagtaaggagcaagaagagctgatccggacactcctgggggcccacacccgccacatgggcaccatgtttgaacagtttgtgcagtttaggcctccagctcatctgttcatccatcaccagcccttgcccaccctggcccctgtgctgcctctggtcacacacttcgcagacatcaacactttcatggtactgcaagtcatcaagtttactaaggacctgcccgtcttccgttccctgcccattgaagaccagatctcccttctcaagggagcagctgtggaaatctgtcacatcgtactcaataccactttctgtctccaaacacaaaacttcctctgcgggcctcttcgctacacaattgaagatggagcccgtgtggggttccaggtagagtttttggagttgctctttcacttccatggaacactacgaaaactgcagctccaagagcctgagtatgtgctcttggctgccatggccctcttctctcctgaccgacctggagttacccagagagatgagattgatcagctgcaagaggagatggcactgactctgcaaagctacatcaagggccagcagcgaaggccccgggatcggtttctgtatgcgaagttgctaggcctgctggctgagctccggagcattaatgaggcctacgggtaccaaatccagcacatccagggcctgtctgccatgatg

SEQ ID NO: 4 - protein, 238 aa, Homo sapiens
PVQLSKEQEELIRTLLGAHTRHMGTMFEQFVQFRPPAHLFIHHQPLPTLAPVLPLVTHFADINTFMVLQVIKFTKDLPVFRSLPIEDQISLLKGAAVEICHIVLNTTFCLQTQNFLCGPLRYTIEDGARVGFQVEFLELLFHFHGTLRKLQLQEPEYVLLAAMALFSPDRPGVTQRDEIDQLQEEMALTLQSYIKGQQRRPRDRFLYAKLLGLLAELRSINEAYGYQIQHIQGLSAMM

SEQ ID NO: 5 - protein, 11 aa, Artificial
  Amino acid sequence of an artificial His tag
MKKGHHHHHHG

SEQ ID NO: 6 - DNA, 69 bp, Artificial
  Forward primer for amplifying amino acids 103-348, including sequences encoding a His tag, and including an NdeI restriction site
cggcggcgccatatgaaaaaaggtcatcatcatcatcatcatggtcctgtgcaactgagtaaggagcaa

SEQ ID NO: 7 - DNA, 45 bp, Artificial
  Reverse primer for amplifying amino acids 103-348, incorporating a BamHI site
cggcggcgcggatccttattagctgcagatctcctggagcagcgg

(19) United States
(12) Patent Application Publication
(10) Pub. No.: US 2007/0003976 A1
(43) Pub. Date: Jan. 4, 2007

(54) Car ligand-binding domain polypeptide co-crystallized with a ligand, and methods of designing ligands that modulate car activity

(75) Inventors: Jon Loren Collins, Durham, NC (US); Millard Hurst Lambert III, Durham, NC (US); George Bruce Wisely, Durham, NC (US); Xiaoyun Xu, Durham, NC (US)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(73) Assignee: SMITHKLINE BEECHAM CORPORATION, Philadelphia, PA (US)

(21) Appl. No.: 10/565,020

(22) PCT Filed: Jul. 16, 2004
(86) PCT No.: PCT/US04/23092

§ 371 Date: Jan. 18, 2006

Related U.S. Application Data
(60) Provisional application No. 60/488,415, filed on Jul. 18, 2003.

(51) Int. Cl. C40B 30/06; C40B 40/04; C07D 235/28; C07K 14/705

(52) U.S. Cl. 435/7.1; 530/350; 548/307.1

(57) ABSTRACT

The present invention provides a crystalline form of a substantially pure constitutive androstane receptor (CAR) polypeptide. Also provided is a crystalline form of a substantially pure constitutive androstane receptor (CAR) polypeptide in complex with a ligand. Also provided are methods for generating the crystalline forms of the present invention and methods for identifying and designing CAR ligands and modulators. Also provided are scalable three-dimensional configurations of points and computer readable storage media containing digitally encoded structural data.

### TECHNICAL FIELD

The present invention relates generally to the structure of the ligand-binding domain of CAR, and more particularly to the structure of the ligand-binding domain of CAR in complex with a ligand. The present invention also relates to CAR binding compounds and to the design of compounds that bind to CAR.

### Abbreviations

amu—atomic mass unit(s)

ATP—adenosine triphosphate

ADP—adenosine diphosphate

BSA—bovine serum albumin

CaMV—cauliflower mosaic virus

CAR—constitutive androstane receptor

CARαa—constitutive androstane receptor alpha

CBP—CREB binding protein

CCDB—Cambridge Crystallographic Data Bank

cDNA—complementary DNA

CPU—central processing unit

RAM—random access memory

CRT—cathode-ray tube

DBD—DNA binding domain

DMSO—dimethyl sulfoxide

DNA—deoxyribonucleic acid

DTT—dithiothreitol

EDTA—ethylenediaminetetraacetic acid

Et₂O—diethyl ether

FEDs—field emission displays

GST—glutathione S-transferase

HEPES—N-2-hydroxyethylpiperazine-N′-2-ethanesulfonic acid

kDa—kilodalton(s)

LBD—ligand-binding domain

LCDs—liquid crystal displays

LED—light emitting diode

MPD—methyl-pentanediol

MCAR—mouse constitutive androstane receptor

MIR—multiple isomorphous replacement

MPD—methyl pentanediol

N-COR—nuclear co-repressor

NDP—nucleotide diphosphate

NR—nuclear receptor

nt—nucleotide(s)

NTP—nucleotide triphosphate

PAGE—polyacrylamide gel electrophoresis

PCR—polymerase chain reaction

PEG—polyethylene glycol

pI—isoelectric point

PXR—pregnane X receptor

PBREM—phenobarbital-responsive enhancer module

RAR—retinoic acid receptor

RAREs—retinoic acid response elements

rCAR—rat constitutive androstane receptor

RUBISCO—ribulose bisphosphate carboxylase

RXR—retinoid X receptor

SDS—sodium dodecyl sulfate

SDS-PAGE—sodium dodecyl sulfate polyacrylamide gel electrophoresis

SMRT—silencing mediator for retinoid and thyroid receptors

SRC-1—steroid receptor coactivator-1

SR—steroid receptor

TFA—trifluoroacetic acid

TMV—tobacco mosaic virus

TR—thyroid receptor

VDR—vitamin D receptor

Amino Acid Abbreviations, Codes, and
Functionally Equivalent Codons

| | 3- | 1- | |
|---|---|---|---|
| Amino Acid | Letter | Letter | Codons |
| Alanine | Ala | A | GCA GCC GCG GCU |
| Arginine | Arg | R | AGA AGG CGA CGC CGG CGU |
| Asparagine | Asn | N | AAC AAU |
| Aspartic Acid | Asp | D | GAC GAU |
| Cysteine | Cys | C | UGC UGU |
| Glutamic acid | Glu | E | GAA GAG |
| Glutamine | Gln | Q | CAA CAG |
| Glycine | Gly | G | GGA GGC GGG GGU |
| Histidine | His | H | CAC CAU |
| Isoleucine | Ile | I | AUA AUC AUU |
| Leucine | Leu | L | UUA UUG CUA CUC CUG CUU |
| Lysine | Lys | K | AAA AAG |
| Methionine | Met | M | AUG |
| Phenylalanine | Phe | F | UUC UUU |
| Proline | Pro | P | CCA CCC CCG CCU |
| Serine | Ser | S | ACG AGU UCA UCC UCG UCU |
| Threonine | Thr | T | ACA ACC ACG ACU |
| Tryptophan | Trp | W | UGG |
| Tyrosine | Tyr | Y | UAC UAU |
| Valine | Val | V | GUA GUC GUG GUU |

### BACKGROUND

The constitutive androstane receptor (CAR; Unified Nomenclature Committee designation NR1I3) was isolated in 1994 by screening a human liver library with a degenerate oligonucleotide probe based on the P box region (Baes et al., 1994). CAR was subsequently shown to be a heterodimer partner for RXR that acts as a specific, retinoid-independent activator of a subset of retinoic acid response elements (RAREs). The mouse CAR homologue was also isolated in 1994 (Honkakoski et al., 1998). Mouse CAR studies showed that RXR and CAR bind to a site in the phenobarbital-responsive enhancer module (PBREM) of the cytochrome P-450 Cyp2b10 gene in response to phenobarbital induction. Expression of RXR and CAR in mammalian cell lines activated PBREM, indicating that a CAR-RXR heterodimer is a trans-acting factor for the mouse Cyp2b10 gene. These studies were the first to indicate that CAR might play a role in response to xenobiotics.

The ability to respond to a wide range of potentially toxic chemicals is essential in a complex environment. Evidence is accumulating that CAR and its closest mammalian homologue, the pregnane X receptor (PXR; Unified Nomenclature Committee designation NR1I2), evolved to detect xenobiotics as part of the body's detoxification machinery (Waxman, 1999). Both receptors are highly expressed in the liver and intestine and both regulate the expression of specific detoxification genes. PXR and CAR regulate genes whose protein products are involved in the hydroxylation (phase I), conjugation (phase II), and transport of xenobiotics (phase III). CAR is activated by some of the same ligands as PXR (Moore et al., 2000), regulates at least partially overlapping sets of genes (e.g. CYP3A and CYP2B; Xie et al., 2000a), and can signal through the same response elements (Goodwin et al., 2001; Handschin et al., 2001).

Despite these similarities, CAR differs from PXR in several respects. CAR ligand binding has been shown to be more restricted than that of PXR (Moore et al., 2000). Furthermore, CAR displays a high basal level of activity relative to PXR that can be reduced by the binding of either naturally occurring androstanes or xenobiotics such as clotrimazole (Baes et al., 1994; Moore et al., 2000). Finally, CAR displays fundamental differences from PXR with regard to its cellular regulation. In mouse primary hepatocytes and in mouse liver in vivo, CAR is cytoplasmic in the naive state and translocates to the nucleus upon activation (Kawamoto et al., 1999), a process thought to be regulated in part by dephosphorylation of the receptor (Honkakoski et al., 1998). Induction of CAR nuclear translocation does not necessarily depend upon ligand-binding, as phenobarbital has been shown to be an activator of CAR in vivo and in hepatocytes, but does not appear to interact directly with the CAR ligand-binding domain (Moore et al., 2000). Thus, CAR has a high basal level of transcriptional activity even in the absence of an exogenous ligand. An important goal of future efforts will be to further differentiate the physical and functional properties of CAR from PXR, and to ultimately distinguish the unique physiological role of CAR.

Towards this goal, the CAR gene has recently been “knocked-out” by targeted gene disruption (Xie et al., 2000b). The loss of CAR expression did not result in any overt phenotype. Homozygous CAR^{-/-} animals were born at the expected Mendelian frequency, and both male and female CAR-deficient animals were fertile. It was further demonstrated that the nuclear receptor CAR mediates the Cyp2b10 gene response evoked by phenobarbital-like inducers, as well as by the more potent TCPOBOP compound (Xie et al., 2000b). When challenged, these animals showed decreased metabolism of the classic CYP substrate zoxazolamine and a complete loss of the liver hypertrophic and hyperplastic responses to these compounds. These experiments were thus consistent with the notion that at least one aspect of the physiological role of CAR involves xenobiotic metabolism.

Further insight into CAR is expected to be gleaned from CAR structural studies. The availability of the CAR structure will allow an understanding of ligand modulation of CAR activity and will facilitate the design of novel CAR ligands. The present invention addresses these and other needs in the art.

### SUMMARY OF THE INVENTION

The present invention provides a crystalline form comprising a substantially pure constitutive androstane receptor (CAR) ligand-binding domain polypeptide. In one embodiment, the crystalline form comprises a substantially pure constitutive androstane receptor (CAR) ligand-binding domain polypeptide in complex with a ligand. In one embodiment, a ligand is 2-(benzhydrylamino)-1-(2-phenylethyl)-1H-benzimidazole-6-carboxamide.

The present invention also provides a method of generating a crystalline form comprising a constitutive androstane receptor (CAR) ligand-binding domain polypeptide in complex with a ligand, the method comprising: (a) incubating a solution comprising a constitutive androstane receptor (CAR) ligand-binding domain and a ligand with an equal volume of reservoir; and (b) crystallizing the constitutive androstane receptor (CAR) ligand-binding domain polypeptide and ligand using the hanging drop method, whereby a crystalline form of a constitutive androstane receptor (CAR) ligand-binding domain polypeptide in complex with a ligand is generated. Also provided is a crystalline form formed by the above-recited method. In one embodiment, a ligand is 2-(benzhydrylamino)-1-(2-phenylethyl)-1H-benzimidazole-6-carboxamide.

The present invention also provides a method of designing a chemical compound that modulates the biological activity of a target constitutive androstane receptor (CAR) polypeptide. In one embodiment, the method comprises: obtaining one or more three-dimensional structures for the ligand-binding domain (LBD) of constitutive androstane receptor (CAR) in a repressed conformation, and one or more three-dimensional structures of the LBD of constitutive androstane receptor (CAR) in an activated conformation; rotating and translating the three-dimensional structures as rigid bodies so as to superimpose corresponding backbone atoms of a core region of the constitutive androstane receptor (CAR) LBD; comparing one or both of: (i) the superimposed three-dimensional structures to identify volume near the ligand-binding pocket of the constitutive androstane receptor (CAR) LBD that is available to a ligand in the one or more activated structures, or in one or more repressed structures, but that is not available to the ligand in one or more structures of the opposite class; and (ii) the superimposed three-dimensional structures to identify interactions that a ligand could make in one or more of the activated structures, or in one or more of the repressed structures, but which the ligand could not make in one or more structures of the opposite class; and designing a chemical compound that occupies the volume, makes the interaction, or both occupies the volume and makes the interaction.

Optionally the method further comprises synthesizing the designed chemical compound; and testing the designed chemical compound in a biological assay to determine whether it acts as a ligand of constitutive androstane receptor (CAR) with an effect on constitutive androstane receptor (CAR) biological activities, whereby a ligand of a constitutive androstane receptor (CAR) polypeptide is designed.

In another embodiment, the volume or interaction is available in one or more of the repressed structures of constitutive androstane receptor (CAR), but not available in one or more of the activated structures of constitutive androstane receptor (CAR). In another embodiment, the method further comprises designing a chemical compound that promotes the binding of co-repressor to the constitutive androstane receptor (CAR) LBD by making direct favorable interactions with the co-repressor. In another embodiment, the method further comprises designing a chemical compound that reduces binding of a co-repressor to the constitutive androstane receptor (CAR) LBD by making direct unfavorable interactions with the co-repressor. In another embodiment, the method further comprises designing a chemical compound that promotes coactivator binding by displacing an AF2 helix of the constitutive androstane receptor (CAR) LBD and making direct favorable interactions with a coactivator, where the designing allows for an expected movement of the coactivator within a coactivator/co-repressor binding pocket. In yet another embodiment, the method further comprises designing a chemical compound by considering a known agonist of the constitutive androstane receptor (CAR) and adding a substituent that protrudes into the volume identified in step (c) or that makes a desired interaction.

The present invention also provides a binding site in a human constitutive androstane receptor (CAR) polypeptide for a constitutive androstane receptor ligand, wherein the ligand is in van der Waals, hydrogen binding, or van der Waals and hydrogen binding contact with at least one residue of the human constitutive androstane receptor polypeptide.

The present invention also provides a complex of a human constitutive androstane receptor (CAR) ligand-binding domain and a ligand, wherein the ligand is in van der Waals, hydrogen bonding, or both van der Waals and hydrogen bonding contact with at least one of the following residues of the human constitutive androstane receptor polypeptide: Phe161, Ile164, Asn165, Val199, His203, Phe217, Trp224, Thr225, Ile226, Asp228, Gly229, Gln234, Phe238, Leu239, Leu242, Phe243, Tyr326, Met339, Met340.

The present invention also provides a crystal of a complex of a human constitutive androstane receptor (CAR) ligand-binding domain and a ligand, wherein the ligand is in van der Waals, hydrogen bonding, or both van der Waals and hydrogen bonding contact with at least one of the following residues of the human constitutive androstane receptor polypeptide: Phe161, Ile164, Asn165, Val199, His203, Phe217, Trp224, Thr225, Ile226, Asp228, Gly229, Gln234, Phe238, Leu239, Leu242, Phe243, Tyr326, Met339, Met340. In one embodiment, the constitutive androstane receptor is a human constitutive androstane receptor and the crystal has the following physical measurements: space group P2₁2₁2₁ and unit cell: a=83.0 angstroms, b=116.8 angstroms, c=131.9 angstroms, and α=β=γ=90 degrees.

The present invention also provides a method for designing a ligand of a constitutive androstane receptor (CAR) polypeptide, the method comprising: (a) forming a complex of a compound bound to the constitutive androstane receptor (CAR) polypeptide; (b) determining a structural feature of the complex formed in (a); wherein the structural feature is of a binding site for the compound; and (c) using the structural feature determined in (b) to design a ligand of a constitutive androstane receptor (CAR) polypeptide capable of binding to the binding site of the present invention. In one embodiment, the method of the present invention further comprises using a computer-based model of the complex formed in (a) in designing the ligand.

The present invention also provides a method of designing a ligand that selectively modulates the activity of a constitutive androstane receptor (CAR) polypeptide, the method comprising: (a) evaluating a three-dimensional structure of a crystallized constitutive androstane receptor (CAR) ligand-binding domain polypeptide in complex with a ligand; and (b) synthesizing a potential ligand based on the three-dimensional structure of the crystallized constitutive androstane receptor (CAR) catalytic polypeptide in complex with a ligand, whereby a ligand that selectively modulates the activity of a constitutive androstane receptor (CAR) polypeptide is designed. In one embodiment, the constitutive androstane receptor (CAR) ligand-binding domain polypeptide comprises the amino acid sequence of SEQ ID NO: 4. In one embodiment, the crystalline form is such that the three-dimensional structure of the crystallized constitutive androstane receptor (CAR) ligand-binding domain polypeptide in complex with a ligand can be determined to a resolution of about 2.15 Å or better. In one embodiment, the method further comprises contacting a constitutive androstane receptor (CAR) ligand-binding domain polypeptide with the potential ligand and a ligand; and assaying the constitutive androstane receptor (CAR) ligand-binding domain polypeptide for binding of the potential ligand, for a change in activity of the constitutive androstane receptor (CAR) ligand-binding domain polypeptide, or both. In one embodiment, the ligand is 2-(benzhydrylamino)-1-(2-phenylethyl)-1H-benzimidazole-6-carboxamide.

The present invention also provides a method of screening a plurality of compounds for a ligand of a constitutive androstane receptor (CAR) ligand-binding domain polypeptide, the method comprising: (a) providing a library of test samples; (b) contacting a crystalline form comprising a constitutive androstane receptor (CAR) polypeptide in complex with a ligand with each test sample; (c) detecting an interaction between a test sample and the crystalline constitutive androstane receptor (CAR) polypeptide in complex with a ligand; (d) identifying a test sample that interacts with the crystalline constitutive androstane receptor (CAR) polypeptide in complex with a ligand; and (e) isolating a test sample that interacts with the crystalline constitutive androstane receptor (CAR) polypeptide in complex with a ligand, whereby a plurality of compounds is screened for a ligand of a constitutive androstane receptor (CAR) ligand-binding domain polypeptide. In one embodiment, the CAR polypeptide comprises a CAR ligand-binding domain. In another embodiment, the CAR polypeptide is a human CAR polypeptide. In yet another embodiment, the CAR polypeptide comprises the amino acid sequence of SEQ ID NO: 4. In one embodiment, the library of test samples is bound to a substrate. In another embodiment, the library of test samples is synthesized directly on a substrate. In one embodiment, the ligand is 2-(benzhydrylamino)-1-(2-phenylethyl)-1H-benzimidazole-6-carboxamide,

The present invention also provides a method for identifying a constitutive androstane receptor (CAR) ligand, the method comprising: (a) providing atomic coordinates of a constitutive androstane receptor (CAR) ligand-binding domain in complex with a ligand to a computerized modeling system; and (b) modeling a ligand that fits spatially into the binding pocket of the constitutive androstane receptor (CAR) ligand-binding domain to thereby identify a constitutive androstane receptor (CAR) ligand. In one embodiment, the method further comprises identifying in an assay for constitutive androstane receptor (CAR)-mediated activity a modeled ligand that increases or decreases the activity of the constitutive androstane receptor (CAR). In one embodiment, the CAR is a human CAR. In one embodiment, the CAR ligand-binding domain comprises the amino acid sequence of SEQ ID NO: 4. In one embodiment, the ligand is 2-(benzhydrylamino)-1-(2-phenylethyl)-1H-benzimidazole-6-carboxamide.

The present invention also provides a method of identifying a constitutive androstane receptor (CAR) ligand that selectively binds a constitutive androstane receptor (CAR) polypeptide compared to other polypeptides, the method comprising: (a) providing atomic coordinates of a constitutive androstane receptor (CAR) ligand-binding domain in complex with a ligand to a computerized modeling system; and (b) modeling a ligand that fits into the binding pocket of a constitutive androstane receptor (CAR) ligand-binding domain and that interacts with residues of a constitutive androstane receptor (CAR) ligand-binding domain that are conserved among constitutive androstane receptor (CAR) subtypes to thereby identify a constitutive androstane receptor (CAR) ligand that selectively binds a constitutive androstane receptor (CAR) polypeptide compared to other polypeptides. In one embodiment, the method further comprises identifying in a biological assay for constitutive androstane receptor (CAR) activity a modeled ligand that selectively binds to said constitutive androstane receptor (CAR) and increases or decreases the activity of the constitutive androstane receptor (CAR). In one embodiment, the CAR ligand-binding domain comprises the amino acid sequence shown in SEQ ID NO: 4. In one embodiment, the ligand is 2-(benzhydrylamino)-1-(2-phenylethyl)-1H-benzimidazole-6-carboxamide.

The present invention also provides a method of designing a ligand of a constitutive androstane receptor (CAR) polypeptide, the method comprising: (a) selecting a candidate constitutive androstane receptor (CAR) ligand; (b) determining which amino acid or amino acids of a constitutive androstane receptor (CAR) polypeptide interact with the ligand using a three-dimensional model of a crystallized protein, the model comprising a constitutive androstane receptor (CAR) ligand-binding domain in complex with a ligand; (c) identifying in a biological assay for constitutive androstane receptor (CAR) activity a degree to which the ligand modulates the activity of the constitutive androstane receptor (CAR) polypeptide; (d) selecting a chemical modification of the ligand wherein the interaction between the amino acids of the constitutive androstane receptor (CAR) polypeptide and the ligand is predicted to be modulated by the chemical modification; (e) synthesizing a ligand having the chemical modified to form a modified ligand; (f) contacting the modified ligand with the constitutive androstane receptor (CAR) polypeptide; (g) identifying in a biological assay for constitutive androstane receptor (CAR) activity a degree to which the modified ligand modulates the biological activity of the constitutive androstane receptor (CAR) polypeptide; and (h) comparing the biological activity of the constitutive androstane receptor (CAR) polypeptide in the presence of modified ligand with the biological activity of the constitutive androstane receptor (CAR) polypeptide in the presence of the unmodified ligand, whereby a ligand of a constitutive androstane receptor (CAR) polypeptide is designed. In one embodiment, wherein the method further comprises repeating steps (a) through (f), if the biological activity of the constitutive androstane receptor (CAR) polypeptide in the presence of the modified ligand varies from the biological activity of the constitutive androstane receptor (CAR) polypeptide in the presence of the unmodified ligand.

The present invention also provides a crystallized, recombinant polypeptide comprising: (a) an amino acid sequence set forth in SEQ ID NO: 2 or SEQ ID NO: 4; (b) an amino acid sequence having at least about 95% identity with the amino acid sequence set forth in SEQ ID NO: 2 or SEQ ID NO: 4; or (c) an amino acid sequence encoded by a polynucleotide that hybridizes under stringent conditions to the complementary strand of a polynucleotide having SEQ ID NO: 1 or SEQ ID NO: 3 and has at least one biological activity of constitutive androstane receptor (CAR); wherein the polypeptide of (a), (b) or (c) is in crystal form. In one embodiment, the crystallized, recombinant polypeptide diffracts X-rays to a resolution of about 2.5 Å or better. In another embodiment, the polypeptide comprises at least one heavy atom label. In another embodiment, the polypeptide is labeled with seleno-methionine.

The present invention also provides a method for designing a modulator for the prevention or treatment of a disease or disorder, comprising: (a) providing a three-dimensional structure for a crystallized, recombinant polypeptide; (b) identifying a potential modulator for the prevention or treatment of a disease or disorder by reference to the three-dimensional structure; (c) contacting a polypeptide or a constitutive androstane receptor (CAR) with the potential modulator; and (d) assaying the activity of the polypeptide after contact with the modulator, wherein a change in the activity of the polypeptide indicates that the modulator can be useful for prevention or treatment of a disease or disorder.

The present invention also provides a method for obtaining structural information of a crystallized polypeptide, the method comprising: (a) crystallizing a recombinant polypeptide, wherein the polypeptide comprises: (1) an amino acid sequence set forth in SEQ ID NO: 2 or SEQ ID NO: 4; (2) an amino acid sequence having at least about 95% identity with the amino acid sequence set forth in SEQ ID NO: 2 or SEQ ID NO: 4; or (3) an amino acid sequence encoded by a polynucleotide that hybridizes under stringent conditions to the complementary strand of a polynucleotide having SEQ ID NO: 1 or SEQ ID NO: 3 and has at least one biological activity of human constitutive androstane receptor (CAR); and wherein the crystallized polypeptide is capable of diffracting X-rays to a resolution of 2.5 Å or better; and (b) analyzing the crystallized polypeptide by X-ray diffraction to determine the three-dimensional structure of at least a portion of the crystallized polypeptide. In one embodiment, the three-dimensional structure of the portion of the crystallized polypeptide is determined to a resolution of 2.5 Å or better.

The present invention also provides a method for identifying a druggable region of a polypeptide, the method comprising: (a) obtaining crystals of a polypeptide comprising (1) an amino acid sequence set forth in SEQ ID NO: 2 or SEQ ID NO: 4; (2) an amino acid sequence having at least about 95% identity with the amino acid sequence set forth in SEQ ID NO: 2 or SEQ ID NO: 4; or (3) an amino acid sequence encoded by a polynucleotide that hybridizes under stringent conditions to the complementary strand of a polynucleotide having SEQ ID NO: 1 or SEQ ID NO: 3 and has at least one biological activity of human constitutive androstane receptor (CAR), such that the three dimensional structure of the crystallized polypeptide can be determined to a resolution of 2.5 Å or better; (b) determining the three dimensional structure of the crystallized polypeptide using X-ray diffraction; and (c) identifying a druggable region of the crystallized polypeptide based on the three-dimensional structure of the crystallized polypeptide. In one embodiment, the druggable region is an active site. In another embodiment, the druggable region is on the surface of the polypeptide.

The present invention also provides a crystalline human constitutive androstane receptor (CAR) comprising a crystal having unit cell dimensions a=83.0 Å; b=116.8 Å; c=131.9 Å; α=β=γ=90°; with an orthorhombic space group P2₁2₁2₁ and 4 molecules per asymmetric unit.

The present invention also provides a crystallized polypeptide comprising: (1) an amino acid sequence set forth in SEQ ID NO: 2 or SEQ ID NO: 4; (2) an amino acid sequence having at least about 95% identity with the amino acid sequence set forth in SEQ ID NO: 2 or SEQ ID NO: 4; or (3) an amino acid sequence encoded by a polynucleotide that hybridizes under stringent conditions to the complementary strand of a polynucleotide having SEQ ID NO: 1 or SEQ ID NO: 3 and has at least one biological activity of human constitutive androstane receptor (CAR); wherein the crystal has a P2₁2₁2₁ space group.

The present invention also provides a crystallized polypeptide comprising a structure of a polypeptide that is defined by a substantial portion of the atomic coordinates set forth in Table 2 or Table 3.

The present invention also provides a method for determining the crystal structure of a homolog of a polypeptide, the method comprising: (a) providing the three dimensional structure of a first crystallized polypeptide comprising (1) an amino acid sequence set forth in SEQ ID NO: 2 or SEQ ID NO: 4; (2) an amino acid sequence having at least about 95% identity with the amino acid sequence set forth in SEQ ID NO: 2 or SEQ ID NO: 4; or (3) an amino acid sequence encoded by a polynucleotide that hybridizes under stringent conditions to the complementary strand of a polynucleotide having SEQ ID NO: 1 or SEQ ID NO: 3 and has at least one biological activity of human constitutive androstane receptor (CAR); (b) obtaining crystals of a second polypeptide comprising an amino acid sequence that is at least 70% identical to the amino acid sequence set forth in SEQ ID NO: 2 or SEQ ID NO: 4, such that the three dimensional structure of the second crystallized polypeptide can be determined to a resolution of 2.5 Å or better; and (c) determining the three dimensional structure of the second crystallized polypeptide by X-ray crystallography based on the atomic coordinates of the three dimensional structure provided in step (a). In one embodiment, the atomic coordinates for the second crystallized polypeptide have a root mean square deviation from the backbone atoms of the first polypeptide of not more than 1.5 Å for all backbone atoms shared in common with the first polypeptide and the second polypeptide.

The present invention also provides a method for homology modeling a homolog of human constitutive androstane receptor (CAR), comprising: (a) aligning the amino acid sequence of a homolog of human constitutive androstane receptor (CAR) with an amino acid sequence of SEQ ID NO: 2 or SEQ ID NO: 4 and incorporating the sequence of the homolog of human CAR into a model of human constitutive androstane receptor (CAR) derived from structure coordinates as listed in Table 2 or Table 3 to yield a preliminary model of the homolog of human CAR; (b) subjecting the preliminary model to energy minimization to yield an energy minimized model; (c) remodeling regions of the energy minimized model where stereochemistry restraints are violated to yield a final model of the homolog of human constitutive androstane receptor (CAR).

The present invention also provides a method for obtaining structural information about a molecule or a molecular complex of unknown structure comprising: (a) crystallizing the molecule or molecular complex; (b) generating an X-ray diffraction pattern from the crystallized molecule or molecular complex; (c) applying at least a portion of the structure coordinates set forth in Table 2 or Table 3 to the X-ray diffraction pattern to generate a three-dimensional electron density map of at least a portion of the molecule or molecular complex whose structure is unknown.

The present invention also provides a method for attempting to make a crystallized complex comprising a polypeptide and a modulator having a molecular weight of less than 5 kDa, the method comprising: (a) crystallizing a polypeptide comprising (1) an amino acid sequence set forth in SEQ ID NO: 2 or SEQ ID NO: 4; (2) an amino acid sequence having at least about 95% identity with the amino acid sequence set forth in SEQ ID NO: 2 or SEQ ID NO: 4; or (3) an amino acid sequence encoded by a polynucleotide that hybridizes under stringent conditions to the complementary strand of a polynucleotide having SEQ ID NO: 1 or SEQ ID NO: 3 and has at least one biological activity of human constitutive androstane receptor (CAR); such that crystals of the crystallized polypeptide will diffract X-rays to a resolution of 5 Å or better; and (b) soaking the crystals in a solution comprising a potential modulator having a molecular weight of less than 5 kDa.

The present invention also provides a method for incorporating a potential modulator in a crystal of a polypeptide, comprising placing a hexagonal crystal of human constitutive androstane receptor (CAR) having unit cell dimensions a=83.0 Å; b=116.8 Å; c=131.9 Å, a=b=g=90°, with an orthorhombic space group P212121, in a solution comprising the potential modulator.

The present invention also provides a computer readable storage medium comprising digitally encoded structural data, wherein the data comprises structural coordinates as listed in Table 2 or Table 3 for the backbone atoms of at least about six amino acid residues from a druggable region of human constitutive androstane receptor (CAR).

The present invention also provides a scalable three-dimensional configuration of points, at least a portion of the points derived from some or all of the structure coordinates as listed in Table 2 or Table 3 for a plurality of amino acid residues from a druggable region of human constitutive androstane receptor (CAR). In one embodiment, the structure coordinates as listed in Table 2 or Table 3 for the backbone atoms of at least about five amino acid residues from a druggable region of human constitutive androstane receptor (CAR) are used to derive part or all of the portion of points. In another embodiment, the structure coordinates as listed in Table 2 or Table 3 for the backbone and optionally the side chain atoms of at least about ten amino acid residues from a druggable region of human constitutive androstane receptor (CAR) are used to derive part or all of the portion of points. In another embodiment, the structure coordinates as listed in Table 2 or Table 3 for the backbone atoms of at least about fifteen amino acid residues from a druggable region of human constitutive androstane receptor (CAR) are used to derive part or all of the portion of points. In another embodiment, substantially all of the points are derived from structure coordinates as listed in Table 2 or Table 3. In still another embodiment, the structure coordinates as listed in Table 2 or Table 3 for the atoms of the amino acid residues from any of the above-described druggable regions of human constitutive androstane receptor (CAR) are used to derive part or all of the portion of points.

The present invention also provides a scalable three-dimensional configuration of points, comprising points having a root mean square deviation of less than about 1.5 Å from the three dimensional coordinates as listed in Table 2 or Table 3 for the backbone atoms of at least five amino acid residues, wherein the five amino acid residues are from a druggable region of human constitutive androstane receptor (CAR). In one embodiment, any point-to-point distance, calculated from the three dimensional coordinates as listed in Table 2 or Table 3, between one of the backbone atoms for one of the five amino acid residues and another backbone atom of a different one of the five amino acid residues is not more than about 10 Å.

The present invention also provides a scalable three-dimensional configuration of points comprising points having a root mean square deviation of less than about 1.5 Å from the three dimensional coordinates as listed in Table 2 or Table 3 for the atoms of the amino acid residues from any of the above-described druggable regions of human constitutive androstane receptor (CAR).

The present invention also provides a computer readable storage medium comprising digitally encoded structural data, wherein the data comprise the identity and three-dimensional coordinates as listed in Table 2 or Table 3 for the atoms of the amino acid residues from any of the above-described druggable regions of human constitutive androstane receptor (CAR).

The present invention also provides a scalable three-dimensional configuration of points, wherein the points have a root mean square deviation of less than about 1.5 Å from the three dimensional coordinates as listed in Table 2 or Table 3 for the atoms of the amino acid residues from any of the above-described druggable regions of human constitutive androstane receptor (CAR), wherein up to one amino acid residue in each of the regions can have a conservative substitution thereof.

The present invention also provides a scalable three-dimensional configuration of points derived from a druggable region of a polypeptide, wherein the points have a root mean square deviation of less than about 1.5 Å from the three dimensional coordinates as listed in Table 2 or Table 3 for the backbone atoms of at least ten amino acid residues that participate in the intersubunit contacts of human constitutive androstane receptor (CAR).

The present invention also provides a computer-assisted method for identifying an inhibitor of the activity of human constitutive androstane receptor (CAR), comprising: (a) supplying a computer modeling application with a set of structure coordinates as listed in Table 2 or Table 3 for the atoms of the amino acid residues from any of the above-described druggable regions of human constitutive androstane receptor (CAR) so as to define part or all of a molecule or complex; (b) supplying the computer modeling application with a set of structure coordinates of a chemical entity; and (c) determining whether the chemical entity is expected to bind to or interfere with the molecule or complex. In one embodiment, determining whether the chemical entity is expected to bind to or interfere with the molecule or complex comprises performing a fitting operation between the chemical entity and a druggable region of the molecule or complex, followed by computationally analyzing the results of the fitting operation to quantify the association between the chemical entity and the druggable region. In one embodiment, the method further comprises screening a library of chemical entities.

The present invention also provides a computer-assisted method for designing an inhibitor of constitutive androstane receptor (CAR) activity comprising: (a) supplying a computer modeling application with a set of structure coordinates having a root mean square deviation of less than about 1.5 Å from the structure coordinates as listed in Table 2 or Table 3 for the atoms of the amino acid residues from any of the above-described druggable regions of human constitutive androstane receptor (CAR) so as to define part or all of a molecule or complex; (b) supplying the computer modeling application with a set of structure coordinates for a chemical entity; (c) evaluating the potential binding interactions between the chemical entity and the molecule or complex; (d) structurally modifying the chemical entity to yield a set of structure coordinates for a modified chemical entity; and (e) determining whether the modified chemical entity is an inhibitor expected to bind to or interfere with the molecule or complex, wherein binding to or interfering with the molecule or molecular complex is indicative of potential inhibition of constitutive androstane receptor (CAR) activity. In one embodiment, determining whether the modified chemical entity is an inhibitor expected to bind to or interfere with the molecule or complex comprises performing a fitting operation between the chemical entity and the molecule or complex, followed by computationally analyzing the results of the fitting operation to evaluate the association between the chemical entity and the molecule or complex. In another embodiment, the set of structure coordinates for the chemical entity is obtained from a chemical library.

The present invention also provides a computer-assisted method for designing an inhibitor of constitutive androstane receptor (CAR) activity de novo comprising: (a) supplying a computer modeling application with a set of three-dimensional coordinates derived from the structure coordinates as listed in Table 2 or Table 3 for the atoms of the amino acid residues from any of the above-described druggable regions of human constitutive androstane receptor (CAR) so as to define part or all of a molecule or complex; (b) computationally building a chemical entity represented by a set of structure coordinates; and (c) determining whether the chemical entity is an inhibitor expected to bind to or interfere with the molecule or complex, wherein binding to or interfering with the molecule or complex is indicative of potential inhibition of constitutive androstane receptor (CAR) activity. In one embodiment, determining whether the chemical entity is an inhibitor expected to bind to or interfere with the molecule or complex comprises performing a fitting operation between the chemical entity and a druggable region of the molecule or complex, followed by computationally analyzing the results of the fitting operation to quantify the association between the chemical entity and the druggable region.

The present invention also provides a method for identifying a potential modulator for the prevention or treatment of a disease or disorder, the method comprising: (a) providing the three dimensional structure of a crystallized polypeptide comprising: (1) an amino acid sequence set forth in SEQ ID NO: 2 or SEQ ID NO: 4; (2) an amino acid sequence having at least about 95% identity with the amino acid sequence set forth in SEQ ID NO: 2 or SEQ ID NO: 4; or (3) an amino acid sequence encoded by a polynucleotide that hybridizes under stringent conditions to the complementary strand of a polynucleotide having SEQ ID NO: 1 or SEQ ID NO: 3 and has at least one biological activity of human constitutive androstane receptor (CAR); (b) obtaining a potential modulator for the prevention or treatment of a disease or disorder based on the three dimensional structure of the crystallized polypeptide; (c) contacting the potential modulator with a second polypeptide comprising: (i) an amino acid sequence set forth in SEQ ID NO: 2 or SEQ ID NO: 4; (ii) an amino acid sequence having at least about 95% identity with the amino acid sequence set forth in SEQ ID NO: 2 or SEQ ID NO: 4; or (iii) an amino acid sequence encoded by a polynucleotide that hybridizes under stringent conditions to the complementary strand of a polynucleotide having SEQ ID NO: 1 or SEQ ID NO: 3 and has at least one biological activity of human constitutive androstane receptor (CAR); which second polypeptide can optionally be the same as the crystallized polypeptide; and (d) assaying the activity of the second polypeptide, wherein a change in the activity of the second polypeptide indicates that the compound can be useful for prevention or treatment of a disease or disorder.

The present invention also provides a method for designing a candidate modulator for screening for inhibitors of a polypeptide, the method comprising: (a) providing the three dimensional structure of a druggable region of a polypeptide comprising (1) an amino acid sequence set forth in SEQ ID NO: 2 or SEQ ID NO: 4; (2) an amino acid sequence having at least about 95% identity with the amino acid sequence set forth in SEQ ID NO: 2 or SEQ ID NO: 4; or (3) an amino acid sequence encoded by a polynucleotide that hybridizes under stringent conditions to the complementary strand of a polynucleotide having SEQ ID NO: 1 or SEQ ID NO: 3 and has at least one biological activity of human constitutive androstane receptor (CAR); and (b) designing a candidate modulator based on the three dimensional structure of the druggable region of the polypeptide.

The present invention also provides a method for identifying a potential modulator of a polypeptide from a database, the method comprising: (a) providing the three-dimensional coordinates for a plurality of the amino acids of a polypeptide comprising (1) an amino acid sequence set forth in SEQ ID NO: 2 or SEQ ID NO: 4; (2) an amino acid sequence having at least about 95% identity with the amino acid sequence set forth in SEQ ID NO: 2 or SEQ ID NO: 4; or (3) an amino acid sequence encoded by a polynucleotide that hybridizes under stringent conditions to the complementary strand of a polynucleotide having SEQ ID NO: 1 or SEQ ID NO: 3 and has at least one biological activity of human constitutive androstane receptor (CAR); (b) identifying a druggable region of the polypeptide; and (c) selecting from a database at least one potential modulator comprising three dimensional coordinates which indicate that the modulator can bind or interfere with the druggable region. In one embodiment, the modulator is a small molecule.

The present invention also provides a method for preparing a potential modulator of a druggable region contained in a polypeptide, the method comprising: (a) using the atomic coordinates for the backbone atoms of at least about six amino acid residues from a polypeptide of SEQ ID NO: 4, with a root mean square deviation from the backbone atoms of the amino acid residues of not more than 1.5 Å, to generate one or more three-dimensional structures of a molecule comprising a druggable region from the polypeptide; (b) employing one or more of the three dimensional structures of the molecule to design or select a potential modulator of the druggable region; and (c) synthesizing or obtaining the modulator.

The present invention also provides an apparatus for determining whether a compound is a potential modulator of a polypeptide, the apparatus comprising: (a) a memory that comprises: (i) the three dimensional coordinates and identities of at least about fifteen atoms from a druggable region of a polypeptide comprising (1) an amino acid sequence set forth in SEQ ID NO: 2 or SEQ ID NO: 4; (2) an amino acid sequence having at least about 95% identity with the amino acid sequence set forth in SEQ ID NO: 2 or SEQ ID NO: 4; or (3) an amino acid sequence encoded by a polynucleotide that hybridizes under stringent conditions to the complementary strand of a polynucleotide having SEQ ID NO: 1 or SEQ ID NO: 3 and has at least one biological activity of human constitutive androstane receptor (CAR); (ii) executable instructions; and (b) a processor that is capable of executing instructions to: (i) receive three-dimensional structural information for a candidate modulator; (ii) determine if the three-dimensional structure of the candidate modulator is complementary to the three dimensional coordinates of the atoms from the druggable region; and (iii) output the results of the determination.

The present invention also provides a method for making an inhibitor of constitutive androstane receptor (CAR) activity, the method comprising chemically or enzymatically synthesizing a chemical entity to yield an inhibitor of constitutive androstane receptor (CAR) activity, the chemical entity having been identified during a computer-assisted process comprising supplying a computer modeling application with a set of structure coordinates of a molecule or complex, the molecule or complex comprising at least a portion of at least one druggable region from human constitutive androstane receptor (CAR); supplying the computer modeling application with a set of structure coordinates of a chemical entity; and determining whether the chemical entity is expected to bind or to interfere with the molecule or complex at a druggable region, wherein binding to or interfering with the molecule or complex is indicative of potential inhibition of constitutive androstane receptor (CAR) activity.

The present invention also provides a computer readable storage medium comprising digitally encoded data, wherein the data comprises structural coordinates for a druggable region that is structurally homologous to the structure coordinates as listed in Table 2 or Table 3 for a druggable region of human constitutive androstane receptor (CAR).

The present invention also provides a computer readable storage medium comprising digitally encoded structural data, wherein the data comprise a majority of the three-dimensional structure coordinates as listed in Table 2 or Table 3. In one embodiment, the computer readable storage medium further comprises the identity of the atoms for the majority of the three-dimensional structure coordinates as listed in Table 2 or Table 3. In another embodiment, the data comprise substantially all of the three-dimensional structure coordinates as listed in Table 2 or Table 3.

The present invention also provides a method for building a model for an activated conformation of a constitutive androstane receptor (CAR), the method comprising: (a) employing coordinates for CAR residues 107 to 332 as shown in Table 2; (b) rotating and translating an X-ray structure of the Vitamin D receptor (VDR), so as to superimpose its core backbone atoms onto corresponding atoms from CAR; (c) combining a superimposed VDR AF2 helix, residues 416423, with residues 107-332 from CAR from step (a), to provide a starting model for residues 107-332 and 341-348 of CAR in the activated conformation; (d) computationally mutating Val418, Leu4l9, Val421, Phe422 and Gly423 in the VDR AF2 helix to corresponding amino acids in a CAR AF2 helix, wherein the corresponding amino acids in the CAR AF2 helix are Leu343, Gln344, Ile346, Cys347 and Ser348, respectively; and (e) adjusting the conformations of the mutated amino acid side chains in residues 343, 344, and 346-348 of the AF2 helix of CAR to avoid overlaps, wherein the adjusting is accomplished by one of manual manipulation and conformational search and energy minimization. In one embodiment, the method further comprises modeling a CAR AF2 linker region, residues 333-340, by using a computational loop modeling technique.

Accordingly, it is an object of the present invention to provide a three-dimensional structure of the ligand-binding domain of CAR in complex with a ligand. The object is achieved in whole or in part by the present invention.

An object of the invention having been stated hereinabove, other objects will be evident as the description proceeds, when taken in connection with the accompanying Drawings and Examples as described hereinbelow.

### BRIEF DESCRIPTION OF THE DRAWINGS

FIG. 1 is a ribbon diagram depicting the secondary structure of CAR LBD bound with ligand. The ligand is shown as ball and stick. Helices are indicated by H followed by the a helix number, and P-strands are indicated by b followed by the β-strand number. The line at the bottom of the figure indicates the scale, and corresponds to 50 angstroms. N refers to the N-terminus and C refers to the C-terminus.

FIG. 2 is a structure-based sequence alignment of the human, mouse, and rat CAR polypeptides with the human PXR polypeptide and the human VDR polypeptide. The residues that make up the α helices are boxed with a light gray line and light gray background. The residues that make up the β sheets are boxed with a darker gray line and darker gray background. The residues within 5 Å of the ligand are individually boxed with a thin black square box. Conserved residues are indicated in bold type.

FIG. 3 depicts the CAR ligand-binding site. CAR amino acids are shown with light and dark gray lines. A ligand is shown in heavy black lines. The hydrogen bonds between CAR amino acids and the ligand are shown with dotted lines. Particular amino acids that are involved in the ligand binding are indicated using one letter code and amino acid number.

FIG. 4 is a stick diagram depicting another view of the ligand-binding site. CAR amino acids are shown with light and dark gray lines. A ligand is shown in heavy black lines. The hydrogen bonds between CAR amino acids and the ligand are shown with dotted lines. Particular amino acids that are involved in the ligand binding are indicated using one letter code and amino acid number.

FIG. 5 depicts the CAR binding pocket. Ligand Compound 1 is shown in Van der Walls ball form. The binding pocket is shown as a dotted surface. The protein backbone is shown in ribbon form. The side chains in the binding pocket are shown in ball and stick form.

FIG. 6 depicts another view of the ribbon diagram depicting secondary structure of the three-layer sandwich shaped ligand-binding pocket.

FIG. 7 is a schematic diagram of a general strategy for synthesizing ligands that can bind to the CAR LBD. This scheme is described in Example 6, which outlines the synthesis of an exemplary ligand, Compound 1.

### BRIEF DESCRIPTION OF THE SEQUENCES IN THE SEQUENCE LISTING

SEQ ID NO: 1 is a DNA sequence encoding a full-length human CAR polypeptide.

SEQ ID NO: 2 is an amino acid sequence of a full-length human CAR polypeptide.

SEQ ID NO: 3 is a DNA sequence encoding human CAR residues 103-340, the ligand-binding domain of CAR polypeptide.

SEQ ID NO: 4 is an amino acid sequence of residues 103-340, the ligand-binding domain of CAR polypeptide.

SEQ ID NO: 5 is a His tag amino acid sequence.

SEQ ID NO: 6 is a DNA sequence of a primer used in combination with the primer of SEQ ID NO: 7 to amplify a DNA fragment encoding amino acid residues 103-348 of a human CAR polypeptide. In addition to amplifying these coding nucleotides, the primer also includes sequences that will result in the amplified product (a) encoding a His tag as in SEQ ID NO: 5; and (b) having an NdeI endonuclease restriction site (CATATG) just 5′ to the His tag-encoding residues.

SEQ ID NO: 7 is a DNA sequence of a primer used in combination with the primer of SEQ ID NO: 6 to amplify a DNA fragment encoding residues 103-348 of a human CAR polypeptide. The sequence of this primer includes a BamHI endonuclease restriction site (GGATCC) 3′ to the human CAR polypeptide coding residues. When this primer is used in combination with the primer of SEQ ID NO: 6, the amplified product will have the following arrangement of features: NdeI site—His tag—nucleotides encoding human CAR amino acids 103 to 348—BamHI site.

### DETAILED DESCRIPTION OF THE INVENTION

Until disclosure of the present invention presented herein, the ability to obtain crystalline forms of a CAR LBD, particularly in complex with an antagonist ligand, has not been realized. And until disclosure of the present invention presented herein, a detailed three-dimensional crystal structure of an unliganded CAR polypeptide or a CAR polypeptide in complex with a ligand has not been solved.

In addition to providing structural information, crystalline polypeptides provide other advantages. For example, the crystallization process itself further purifies the polypeptide, and satisfies one of the classical criteria for homogeneity. In fact, crystallization frequently provides unparalleled purification quality, removing impurities that are not removed by other purification methods such as HPLC, dialysis, conventional column chromatography, etc. Moreover, crystalline polypeptides are often stable at ambient temperatures and free of protease contamination and degradation associated with solution storage. Crystalline polypeptides can also be useful as pharmaceutical preparations. Finally, crystallization techniques are generally free of problems such as denaturation associated with other stabilization methods (e.g., lyophilization).

Once crystallization has been accomplished, crystallographic data provides useful structural information that can assist the design of compounds that can serve as agonists or antagonists, as described herein below. In addition, the crystal structure provides information that can be used to map the molecular surface of the ligand-binding domain of CAR. A small non-peptide molecule designed to mimic portions of this surface could serve as a modulator of CAR activity.

I. Definitions

Before the present proteins, nucleotide sequences, and methods are described, it is understood that this invention is not limited to the particular methodology, protocols, cell lines, vectors, and reagents described, as these can vary. It is also to be understood that the terminology used herein is for the purpose of describing particular embodiments only, and is not intended to limit the scope of the present invention, the invention being defined by the claims.

Unless defined otherwise, all technical and scientific terms used herein are intended to have their ordinary meanings as understood by one of ordinary skill in the art to which this invention pertains. Although any methods and materials similar or equivalent to those described herein can be used in the practice or testing of the present invention, representative methods, devices, and materials are now described. All publications mentioned herein are incorporated by reference for the purpose of describing the cell lines, vectors, reagents, and methodologies they disclose.

Following long-standing patent law convention, the articles “a” and “an” are used herein to refer to one or to more than one (i.e., to at least one) of the grammatical object of the article. By way of example, “an element” means one element or more than one element.

As used herein, the term “AF2 helix” refers to a short alpha-helix, usually including 5-8 residues, located at the C-terminal end of a LBD sequence, that can usually adopt multiple positions, orientations, and conformations in the structure, and which is involved in binding to coactivators. In the hypothetical activated conformation of CAR, the AF2 helix is expected to include residues 341 to 347. These residues do not adopt an alpha-helical conformation in the structure of CAR bound to Compound 1.

As used herein, the terms “Compound 1” and “Formula (A)” are used interchangeably and refer to 2-(benzhydrylamino)-1-(2-phenylethyl)-1H-benzimidazole-6-carboxamide.

As used herein, the term “AF2 glutamate” refers to a glutamate residue in the AF2 helix that can make hydrogen bond interactions with the exposed NH groups of the LXXLL-containing peptide from a coactivator if the AF2 helix is in the active position. In CAR, the AF2 glutamate is residue number 345.

As used herein, the terms “activated”, “active conformation”, and “activated conformation” of an LBD are used interchangeably and refer to a conformation where the AF2 helix is in the active position, thereby placing the AF2 glutamate residue in a position and orientation that creates a charge clamp that can recruit coactivator peptides. Similarly, the terms “active position of the AF2 helix” and “active conformation of the AF2 helix” are used interchangeably and mean an AF2 helix having a position and/or orientation similar to that of the AF2 helix in the PPARg/SRC-1/rosiglitazone structure of Nolte et al., 1998, allowing the AF2 glutamate residue to make interactions with the exposed NH groups of a coactivator peptide. The position and/or orientation of the AF2 helix in an NR structure can be compared with that of the AF2 helix in another NR structure by rotating and/or translating one structure so as to superimpose the backbone atoms of helices 1 through 10 onto the corresponding atoms of the other structure, where corresponding residues are determined by sequence alignment. If, after superimposition, a majority of the backbone atoms of the core of the AF2 helix lie within 2.0 angstroms of the corresponding atoms from the PAPRg/SRC-1/rosiglitazone structure, then the AF2 helix is defined as being in an active position or active conformation.

Other examples of a nuclear receptor where the AF2 helix is in an “active position” include the X-ray structures of the estrogen receptor α (ERα) bound to estradiol (Brzozowski et al., 1997) and diethylstilbesterol (DES) (Shiau et al., 1998). Examples of a nuclear receptor where the AF2 helix is not in an “active position” are the X-ray structures of the estrogen receptor a (ERα) bound to raloxifene (Brzozowski et al., 1997) and tamoxifen (Shiau et al., 1998). Binding of a coactivator, and AF2-dependent activation of gene transcription, normally requires that the AF2 helix be in the “active position” (Nolte et al., 1998; Shiau et al., 1998). This creates a “charge-clamp” structure that holds the coactivator in its required position (Nolte et al., 1998).

As used herein, the terms “repressed”, “inactive conformation”, and “repressed conformation” of an LBD are used interchangeably and refer to a conformation where the AF2 helix is not in the active position, and where the AF2 glutamate residue is not in a position that could create the charge clamp that can recruit coactivator peptides.

As used herein, the term “agonist” refers to an agent that supplements or potentiates the biological activity of a functional CAR gene or protein, or of a polypeptide encoded by a gene that is up- or down-regulated by a CAR polypeptide and/or a polypeptide encoded by a gene that contains a CAR binding site or response element in its promoter region. An agent is also an agonist when the changes in gene expression, considered over many genes, are similar in direction to those induced by other agents that are commonly regarded as agonists. In one embodiment, an agonist of CAR is an androstane.

As used herein, the term “antagonist” refers to an agent that decreases or inhibits the biological activity of a functional gene or protein (for example, a functional CAR gene or protein), or that supplements or potentiates the biological activity of a naturally occurring or engineered non-functional gene or protein (for example, a non-functional CAR gene or protein). Alternatively, an antagonist can decrease or inhibit the biological activity of a functional gene or polypeptide encoded by a gene that is up- or down-regulated by a CAR polypeptide and/or contains a CAR binding site or response element in its promoter region. An antagonist can also supplement or potentiate the biological activity of a naturally occurring or engineered non-functional gene or polypeptide encoded by a gene that is up- or down-regulated by a CAR polypeptide, and/or contains a CAR binding site or response element in its promoter region. An agent is also an antagonist when the changes in gene expression, considered over many genes, are opposite in direction to those induced by other agents that are commonly regarded as agonists.

As used herein, the terms “α-helix” and “alpha-helix” are used interchangeably and refer to a conformation of a polypeptide chain wherein the polypeptide backbone is wound around the long axis of the molecule in a left-handed or right-handed direction, and the R groups of the amino acids protrude outward from the helical backbone, wherein the repeating unit of the structure is a single turn of the helix, which extends about 0.56 nm along the long axis.

As used herein, the terms “amino acid”, “amino acid residue”, and “residue” are used interchangeably and refer to an amino acid formed upon chemical digestion (hydrolysis) of a peptide or polypeptide at its peptide linkages. Amino acids can also be synthesized individually or as components of a peptide. In one embodiment, the amino acid residues described herein are in the “L” isomeric form. However, residues in the “D” isomeric form can be substituted for any L-amino acid residue, provided that the desired functional property is retained by the polypeptide. In the context of an amino acid, NH₂ refers to the free amino group present at the amino terminus of a polypeptide, although some amino acids can have NH₂ groups at other positions in the amino acid. COOH refers to the free carboxy group present at the carboxy terminus of a polypeptide. In keeping with standard polypeptide nomenclature, abbreviations for amino acid residues are presented above. The term “amino acid” is intended to embrace all molecules, whether natural or synthetic, which include both an amino functionality and an acid functionality and capable of being included in a polymer of naturally occurring amino acids. Exemplary amino acids include naturally occurring amino acids; analogs, derivatives and congeners thereof; amino acid analogs having variant side chains; and all stereoisomers of any of the foregoing.

It is noted that amino acid residue sequences represented herein by formulae have a left-to-right orientation in the conventional direction of amino terminus to carboxy terminus. In addition, the terms “amino acid”, “amino acid residue”, and “residue” are broadly defined to include the amino acids listed in the above table and modified or unusual amino acids. Furthermore, it is noted that a dash at the beginning or end of an amino acid residue sequence indicates a peptide bond to a further sequence of one or more amino acid residues or a covalent bond to an amino-terminal group such as NH₂ or acetyl or to a carboxy-terminal group such as COOH.

As used herein, the terms “β-sheet” and “beta-sheet” are used interchangeably and refer to the conformation of a polypeptide chain stretched into an extended zigzag conformation. Portions of polypeptide chains that run “parallel” all run in the same direction. Polypeptide chains that are “anti-parallel” run in the opposite direction from the parallel chains or from each other.

The term “binding” refers to an association, which can be a stable association, between two molecules, i.e., between a polypeptide of the invention and a binding partner, due to, for example, electrostatic, hydrophobic, ionic, and/or hydrogen-bond interactions under physiological conditions.

As used herein, the terms “binding pocket of the CAR ligand-binding domain”, “CAR ligand-binding pocket” and “CAR binding pocket” are used interchangeably, and refer to the large cavity within the CAR ligand-binding domain where a ligand (e.g. Compound 1) binds. This cavity can be empty, or can contain water molecules or other molecules from the solvent, or can contain ligand atoms. The “main” binding pocket includes the region of space not occupied by atoms of CAR that is approximately encompassed or bounded by residues Phe132, Phe161, Ile164, Asn165, Thr166, Met168, Val169, Ala198, Val199, Cys202, His203, Leu206, Phe217, Tyr224, Thr225, Ile226, Glu227, Asp228, Gly229, Ala230, Phe234, Phe238, Leu239, Leu242, Phe243, His246, Tyr326, Ile330, Leu336, Ser337, Met339, and Met340. The binding pocket also includes small regions near to and contiguous with the “main” binding pocket that not occupied by atoms of CAR.

As used herein the term “biological activity” refers to any biochemical function of a biological molecule. A biological activity includes, but is not limited to, an interaction with another biological molecule (for example, a polypeptide or a nucleic acid, or a combination thereof). As such, a biological activity results in a biochemical effect including, but not limited to the initiation or inhibition of transcription of a gene.

The term “complex” refers to an association between at least two moieties (i.e. chemical or biochemical) that have an affinity for one another. Examples of complexes include associations between antigen/antibodies, lectin/avidin, target polynucleotide/probe oligonucleotide, antibody/anti-antibody, receptor/ligand, enzyme/ligand, polypeptide/polypeptide, polypeptide/polynucleotide, polypeptide/co-factor, polypeptide/substrate, polypeptide/inhibitor, polypeptide/small molecule, and the like. “Member of a complex” refers to one moiety of the complex, such as an antigen or ligand. “Protein complex” or “polypeptide complex” refers to a complex comprising at least one polypeptide.

The term “conserved residue” refers to an amino acid that is a member of a group of amino acids having certain common properties. The term “conservative amino acid substitution” refers to the substitution (conceptually or otherwise) of an amino acid from one such group with a different amino acid from the same group. A functional way to define common properties between individual amino acids is to analyze the normalized frequencies of amino acid changes between corresponding proteins of homologous organisms (Schulz & Schirmer, 1979). According to such analyses, groups of amino acids can be defined where amino acids within a group exchange preferentially with each other, and therefore resemble each other most in their impact on the overall protein structure (Schulz & Schirmer, 1979). Representative examples of sets of amino acid groups defined in this manner include: (i) a charged group, consisting of Glu and Asp, Lys, Arg and His, (ii) a positively-charged group, consisting of Lys, Arg and His, (iii) a negatively-charged group, consisting of Glu and Asp, (iv) an aromatic group, consisting of Phe, Tyr and Trp, (v) a nitrogen ring group, consisting of His and Trp, (vi) a large aliphatic nonpolar group, consisting of Val, Leu and Ile, (vii) a slightly-polar group, consisting of Met and Cys, (viii) a small-residue group, consisting of Ser, Thr, Asp, Asn, Gly, Ala, Glu, Gln and Pro, (ix) an aliphatic group consisting of Val, Leu, Ile, Met and Cys, and (x) a small hydroxyl group consisting of Ser and Thr.

As used herein, the term “DNA segment” refers to a DNA molecule that has been isolated free of total genomic DNA of a particular species. In one embodiment, a DNA segment encoding a CAR polypeptide refers to a nucleic acid comprising SEQ ID NO: 1. In another embodiment, a DNA segment encoding a CAR polypeptide refers to a nucleic acid comprising SEQ ID NO: 3. DNA segments can comprise a portion of a recombinant vector, including, for example, a plasmid, a cosmid, a phage, a virus, and the like.

As used herein, the term “DNA sequence encoding a CAR polypeptide” refers to one or more coding sequences within a particular individual. Moreover, certain differences in nucleotide sequences can exist between individual organisms, which are called alleles. It is possible that such allelic differences might or might not result in differences in amino acid sequence of the encoded polypeptide yet still encode a protein with the same biological activity. As is well known, genes for a particular polypeptide can exist in single or multiple copies within the genome of an individual. Such duplicate genes can be identical or can have certain modifications, including nucleotide substitutions, additions, or deletions, all of which still code for polypeptides having substantially the same activity.

The term “domain”, when used in connection with a polypeptide, refers to a specific region within the polypeptide that comprises a particular structure or mediates a particular function. In the typical case, a domain of a polypeptide of the invention is a fragment of the polypeptide. In certain instances, a domain is a structurally stable domain, as evidenced, for example, by mass spectroscopy, or by the fact that a modulator can bind to a druggable region of the domain. In one embodiment, a domain of a CAR polypeptide is a ligand-binding domain. In another embodiment, a domain of a CAR polypeptide is a DNA-binding domain.

The term “druggable region”, when used in reference to a polypeptide, nucleic acid, complex and the like, refers to a region of the molecule that is a target or is a likely target for binding a modulator. For a polypeptide, a druggable region generally refers to a region wherein several amino acids of a polypeptide would be capable of interacting with a modulator or other molecule. For a polypeptide or complex thereof, exemplary druggable regions including binding pockets and sites, enzymatic active sites, interfaces between domains of a polypeptide or complex, surface grooves or contours or surfaces of a polypeptide or complex which are capable of participating in interactions with another molecule. In certain instances, the interacting molecule is another polypeptide, which can be naturally occurring. In other instances, the druggable region is on the surface of the molecule. In one embodiment, a druggable region of a CAR polypeptide comprises the binding site defined by amino acid residues 103-340. In another embodiment, a druggable region of a CAR polypeptide comprises amino acid residues and surfaces of the CAR polypeptide that interact with a RXR polypeptide during CAR-RXR heterodimer formation. In another embodiment, a druggable region of a CAR polypeptide comprises the AF2 helix. In another embodiment, a druggable region of a CAR polypeptide comprises Glu345. In still another embodiment, a druggable region of a CAR polypeptide comprises a DNA-binding domain.

Druggable regions can be described and characterized in a number of ways. For example, a druggable region can be characterized by some or all of the amino acids that make up the region, or the backbone atoms thereof, or the side chain atoms thereof (optionally with or without the Cα atoms). Alternatively, in certain instances, the volume of a druggable region corresponds to that of a carbon based molecule of at least about 200 atomic mass units (amu) and often up to about 800 amu. In other instances, it will be appreciated that the volume of such region can correspond to a molecule of at least about 600 amu and often up to about 1600 amu or more.

Alternatively, a druggable region can be characterized by comparison to other regions on the same or other molecules. For example, the term “affinity region” refers to a druggable region on a molecule (such as a polypeptide of the invention) that is present in several other molecules, in so much as the structures of the same affinity regions are sufficiently the same so that they are expected to bind the same or related structural analogs. An example of an affinity region is an ATP-binding site of a protein kinase that is found in several protein kinases (whether or not of the same origin). Another example of an affinity region is a DNA-binding domain: for example, the DNA-binding domain of a CAR polypeptide.

In contrast to an affinity region, the term “selectivity region” refers to a druggable region of a molecule that can not be found on other molecules, in so much as the structures of different selectivity regions are sufficiently different so that they are not expected to bind the same or related structural analogs. An exemplary selectivity region is a catalytic domain of a protein kinase that exhibits specificity for one substrate. In certain instances, a single modulator can bind to the same affinity region across a number of proteins that have a substantially similar biological function, whereas the same modulator can bind to only one selectivity region of one of those proteins.

Continuing with examples of different druggable regions, the term “undesired region” refers to a druggable region of a molecule that upon interacting with another molecule results in an undesirable affect. For example, a binding site that oxidizes the interacting molecule and thereby results in increased toxicity for the oxidized molecule can be deemed an “undesired region”. Other examples of potential undesired regions include regions that upon interaction with a drug decrease the membrane permeability of the drug, increase the excretion of the drug, or increase the blood brain transport of the drug. It can be the case that, in certain circumstances, an undesired region will no longer be deemed an undesired region because the affect of the region will be favorable, i.e., a drug intended to treat a brain condition would benefit from interacting with a region that resulted in increased blood brain transport, whereas the same region could be deemed undesirable for drugs that were not intended to be delivered to the brain.

When used in reference to a druggable region, the “selectivity” or “specificity” of a molecule such as a modulator to a druggable region can be used to describe the binding between the molecule and a druggable region. For example, the selectivity of a modulator with respect to a druggable region can be expressed by comparison to another modulator, using the respective values of K_{d} (i.e., the dissociation constants for each modulator-druggable region complex) or, in cases where a biological effect is observed below the K_{d}, the ratio of the respective EC₅₀'s (i.e., the concentrations that produce 50% of the maximum response for the modulator interacting with each druggable region).

As used herein, the term “expression” generally refers to the cellular processes by which a biologically active polypeptide is produced. As such, the term “expression” generally includes those cellular processes that begin with transcription and end with the production of a functional polypeptide. As used herein, “expression” is also intended to refer to cellular processes by which a polypeptide is produced that would otherwise be functional except for the presence of mutations in the nucleotide sequence encoding it. Consistent with this usage, “expression” includes, but is not limited to, such processes as transcription, translation, post-translational modification, and transport of a polypeptide.

A “fusion protein” or “fusion polypeptide” refers to a chimeric protein as that term is known in the art and can be constructed using methods known in the art. In many examples of fusion proteins, there are two different polypeptide sequences, and in certain cases, there can be more. The sequences can be linked in frame. A fusion protein can include a domain that is found (albeit in a different protein) in an organism that also expresses the first protein, or it can be an “interspecies”, “intergenic”, etc. fusion expressed by different kinds of organisms. In various embodiments, the fusion polypeptide can comprise one or more amino acid sequences linked to a first polypeptide. In the case where more than one amino acid sequence is fused to a first polypeptide, the fusion sequences can be multiple copies of the same sequence, or alternatively, can be different amino acid sequences. The fusion polypeptides can be fused to the N-terminus, the C-terminus, or the N— and C-terminus of the first polypeptide. Exemplary fusion proteins include polypeptides comprising a glutathione S-transferase tag (GST-tag), histidine tag (His-tag), an immunoglobulin domain, or an immunoglobulin-binding domain.

As used herein, the term “gene” is used for simplicity to refer to a nucleotide sequence that encodes a protein, a polypeptide, or a peptide. As such, the term “gene” refers to a nucleic acid comprising an open reading frame encoding a polypeptide having exon sequences and, optionally, intron sequences. The term “intron” refers to a DNA sequence present in a given gene that is not translated into protein and is generally found between exons. As will be understood by those of skill in the art, this functional term includes both genomic sequences and cDNA sequences. Representative embodiments of such sequences are disclosed herein.

The term “having substantially similar biological activity”, when used in reference to two polypeptides, refers to a biological activity of a first polypeptide which is substantially similar to at least one of the biological activities of a second polypeptide. A substantially similar biological activity means that the polypeptides carry out a similar function, i.e., a similar enzymatic reaction or a similar physiological process, etc. For example, two homologous proteins can have a substantially similar biological activity if they are involved in a similar enzymatic reaction, i.e., they are both kinases which catalyze phosphorylation of a substrate polypeptide, however, they can phosphorylate different regions on the same protein substrate or different substrate proteins altogether. Alternatively, two homologous proteins can also have a substantially similar biological activity if they are both involved in a similar physiological process, i.e., regulation of transcription. For example, two proteins can be transcription factors, however, they can bind to different DNA sequences or bind to different polypeptide interactors. Substantially similar biological activities can also be associated with proteins carrying out a similar structural role, for example, two membrane proteins.

As used herein, the term “interact” refers to detectable interactions between molecules, such as can be detected using, for example, a yeast two-hybrid assay. The term “interact” is also meant to include “binding” interactions between molecules. Interactions include, but are not limited to protein-protein, protein-nucleic acid, and protein-small molecule interactions. These interactions can be in the form of covalent or non-covalent interactions including, but not limited to ionic, hydrogen bonding, and van der Waals interactions.

As used herein, the term “isolated” refers to a nucleic acid substantially free of other nucleic acids, proteins, lipids, carbohydrates, or other materials with which it can be associated, such association being either in cellular material or in a synthesis medium. The term can also be applied to polypeptides, in which case the polypeptide is substantially free of nucleic acids, carbohydrates, lipids, and other undesired polypeptides. The term “isolated polypeptide” refers to a polypeptide, in certain embodiments prepared from recombinant DNA or RNA, or of synthetic origin, or some combination thereof, which (1) is not associated with proteins that it is normally found with in nature, (2) is isolated from the cell in which it normally occurs, (3) is isolated free of other proteins from the same cellular source, (4) is expressed by a cell from a different species, or (5) does not occur in nature.

The term “isolated nucleic acid” refers to a polynucleotide of genomic, cDNA, or synthetic origin or some combination there of, which (1) is not associated with the cell in which the “isolated nucleic acid” is found in nature, or (2) is operably linked to a polynucleotide to which it is not linked in nature.

The terms “label” or “labeled” refer to incorporation or attachment, optionally covalently or non-covalently, of a detectable marker into a molecule, such as a polypeptide. Various methods of labeling polypeptides are known in the art and can be used. Examples of labels for polypeptides include, but are not limited to the following: radioisotopes, fluorescent labels, heavy atoms, enzymatic labels or reporter genes, chemiluminescent groups, biotinyl groups, predetermined polypeptide epitopes recognized by a secondary reporter (i.e., leucine zipper pair sequences, binding sites for secondary antibodies, metal binding domains, epitope tags). Examples and use of such labels are well known by the skilled artisan. In some embodiments, spacer arms of various lengths can be attached to labels to reduce potential steric hindrance.

The term “mammal” is known in the art, and exemplary mammals include humans, primates, bovines, porcines, canines, felines, and rodents (i.e., mice and rats).

The term “modulation”, when used in reference to a functional property or biological activity or process (i.e., enzyme activity or receptor binding), refers to the capacity to up regulate (i.e., activate or stimulate), down regulate (i.e., inhibit or suppress), or otherwise change a quality of such property, activity, or process. In certain instances, such regulation can be contingent on the occurrence of a specific event, such as activation of a signal transduction pathway, and/or can be manifest only in particular cell types.

The term “modulator” refers to a polypeptide, nucleic acid, macromolecule, complex, molecule, small molecule, compound, species, or the like (naturally-occurring or non-naturally-occurring), or an extract made from biological materials such as bacteria, plants, fungi, or animal cells or tissues, that can be capable of causing modulation. Modulators can be evaluated for potential activity as inhibitors or activators (directly or indirectly) of a functional property, biological activity or process, or combination thereof, (i.e., agonist, partial antagonist, partial agonist, inverse agonist, antagonist, anti-microbial agents, inhibitors of microbial infection or proliferation, and the like) by inclusion in assays. In such assays, many modulators can be screened at one time. The activity of a modulator can be known, unknown, or partially known.

As used herein, the term “molecular replacement” refers to a method that involves generating a preliminary model of the wild-type CAR ligand-binding domain, or a CAR mutant crystal the structure for which coordinates are unknown, by orienting and positioning a molecule the structure for which coordinates are known (e.g., the vitamin D receptor; VDR) within the unit cell of the unknown crystal so as best to account for the observed diffraction pattern of the unknown crystal. Phases can then be calculated from this model and combined with the observed amplitudes to give an approximate Fourier synthesis of the structure the coordinates for which are unknown. This, in turn, can be subjected to any of the several forms of refinement known in the art to provide a final, accurate structure of the unknown crystal (see e.g. Lattman, 1985; Rossmann, 1972). Using the structure coordinates of the ligand-binding domain of CAR provided by this invention, molecular replacement can be used to determine the structure coordinates of a crystal of a mutant or of a homologue of the CAR ligand-binding domain, or of a different crystal form of the CAR ligand-binding domain.

The term “motif” refers to an amino acid sequence that is commonly found in a protein of a particular structure or function. Typically, a consensus sequence is defined to represent a particular motif. The consensus sequence need not be strictly defined and can contain positions of variability, degeneracy, variability of length, etc. The consensus sequence can be used to search a database to identify other proteins that can have a similar structure or function due to the presence of the motif in its amino acid sequence. For example, on-line databases can be searched with a consensus sequence in order to identify other proteins containing a particular motif. Various search algorithms and/or programs can be used, including FASTA, BLAST, or ENTREZ. FASTA and BLAST are available as a part of the GCG sequence analysis package (Accelrys, Inc., San Diego, Calif., United States of America). ENTREZ is available through the National Center for Biotechnology Information, National Library of Medicine, National Institutes of Health, Bethesda, Md., United States of America.

As used herein, the term “mutation” carries its traditional connotation and refers to a change, inherited, naturally occurring, or introduced, in a nucleic acid or polypeptide sequence, and is used in its sense as generally known to those of skill in the art.

The term “naturally occurring”, as applied to an object, refers to the fact that an object can be found in nature. For example, a polypeptide or polynucleotide sequence that is present in an organism (including bacteria) that can be isolated from a source in nature and which has not been intentionally modified by man in the laboratory is naturally occurring.

The term “nucleic acid” refers to a polymeric form of nucleotides, either ribonucleotides or deoxynucleotides or a modified form of either type of nucleotide. The terms should also be understood to include, as equivalents, analogs of either RNA or DNA made from nucleotide analogs, and, as applicable to the embodiment being described, single-stranded (such as sense or antisense) and double-stranded polynucleotides.

The term “nucleic acid of the invention” refers to a nucleic acid encoding a polypeptide of the invention, i.e., a nucleic acid comprising a sequence consisting of, or consisting essentially of, the polynucleotide sequence set forth in SEQ ID NO: 1 or SEQ ID NO: 3. A nucleic acid of the invention can comprise all, or a portion of: the nucleotide sequence of SEQ ID NO: 1 or SEQ ID NO: 3; a nucleotide sequence at least 60%, 70%, 80%, 90%, 95%, 96%, 97%, 98% or 99% identical to SEQ ID NO: 1 or SEQ ID NO: 3; a nucleotide sequence that hybridizes under stringent conditions to SEQ ID NO: 1 or SEQ ID NO: 3; nucleotide sequences encoding polypeptides that are functionally equivalent to polypeptides of the invention; nucleotide sequences encoding polypeptides at least about 60%, 70%, 80%, 85%, 90%, 95%, 98%, 99% homologous or identical with an amino acid sequence of SEQ ID NO: 2 or SEQ ID NO: 4; nucleotide sequences encoding polypeptides having an activity of a polypeptide of the invention and having at least about 60%, 70%, 80%, 85%, 90%, 95%, 98%, 99% or more homology or identity with SEQ ID NO: 2 or SEQ ID NO: 4; nucleotide sequences that differ by 1 to about 2, 3, 5, 7, 10, 15, 20, 30, 50, 75 or more nucleotide substitutions, additions or deletions, such as allelic variants, of SEQ ID NO: 1 and SEQ ID NO: 3; nucleic acids derived from and evolutionarily related to SEQ ID NO: 1 or SEQ ID NO: 3; and complements of and nucleotide sequences resulting from the degeneracy of the genetic code, for all of the foregoing and other nucleic acids of the invention. Nucleic acids of the invention also include homologs, i.e., orthologs and paralogs, of SEQ ID NO: 1 or SEQ ID NO: 3 and also variants of SEQ ID NO: 1 or SEQ ID NO: 3 which have been codon optimized for expression in a particular organism (i.e., host cell).

The term “operably linked”, when describing the relationship between two nucleic acid regions, refers to a juxtaposition wherein the regions are in a relationship permitting them to function in their intended manner. For example, a control sequence “operably linked” to a coding sequence is ligated in such a way that expression of the coding sequence is achieved under conditions compatible with the control sequences, such as when the appropriate molecules (i.e., inducers and polymerases) are bound to the control or regulatory sequence(s).

As used herein, “orthorhombic unit cell” refers to a unit cell wherein a≠b≠c, and α=β=γ=900. The vectors a, b, and c describe the unit cell edges and the angles α, β, and γ describe the unit cell angles.

As used herein, the term “CAR” refers to any polypeptide with an amino acid sequence that can be aligned with at least one of human, mouse, or rat CAR, such that at least 50% of the amino acids are identical to the corresponding amino acid in the human, mouse, or rat CAR. The term “CAR” also encompasses nucleic acids for which the corresponding translated protein sequence can be considered to be a CAR. The term “CAR” includes vertebrate homologs of CAR family members including, but not limited to mammalian and avian homologs. Representative mammalian homologs of CAR family members include, but are not limited to murine and human homologs.

As used herein, the terms “CAR gene” and “recombinant CAR gene” are used interchangeably and refer to a nucleic acid molecule comprising an open reading frame encoding a CAR polypeptide, including both exon and (optionally) intron sequences.

As used herein, the terms “CAR gene product”, “CAR protein”, “CAR polypeptide”, and “CAR peptide” are used interchangeably and refer to peptides having amino acid sequences which are substantially identical to native CAR amino acid sequences from the organism of interest and which are biologically active in that they comprise all or a part of the amino acid sequence of a CAR polypeptide, or cross-react with antibodies raised against a CAR polypeptide, or retain all or some of the biological activity (e.g., DNA or ligand-binding ability and/or dimerization ability) of the native amino acid sequence or protein. Such biological activity can include immunogenicity.

As used herein, the terms “CAR gene product”, “CAR protein”, “CAR polypeptide”, and “CAR peptide” are used interchangeably and refer to a subtype of the CAR family. In one embodiment, a CAR gene product is CAR. In another embodiment, a CAR gene product comprises the amino acid sequence of SEQ ID NO: 2.

As used herein, the terms “CAR gene product”, “CAR protein”, “CAR polypeptide”, and “CAR peptide” also include analogs of a CAR polypeptide. By “analog” is intended that a DNA or peptide sequence can contain alterations relative to the sequences disclosed herein, yet retain all or some of the biological activity of those sequences. Analogs can be derived from genomic nucleotide sequences as are disclosed herein or those from other organisms, or can be created synthetically. Those skilled in the art will appreciate that other analogs, as yet undisclosed or undiscovered, can be used to design and/or construct CAR analogs. There is no need for a “CAR gene product”, “CAR protein”, “CAR polypeptide”, or “CAR peptide” to comprise all or substantially all of the amino acid sequence of a CAR polypeptide gene product. Shorter or longer sequences are anticipated to be of use in the invention; shorter sequences are herein referred to as “segments”. Thus, the terms “CAR gene product”, “CAR protein”, “CAR polypeptide”, and “CAR peptide” also include fusion or recombinant CAR polypeptides and proteins comprising sequences of the present invention. Methods of preparing such proteins are disclosed herein and are known in the art.

The term “phenotype” refers to the entire physical, biochemical, and physiological makeup of a cell, i.e., having any one trait or any group of traits.

As used herein, the term “polypeptide” refers to any polymer comprising any of the 20 protein amino acids, regardless of its size. Although “protein” is often used in reference to relatively large polypeptides and “peptide” is often used in reference to small polypeptides, usage of these terms in the art overlaps and varies. The term “polypeptide” as used herein refers to peptides, polypeptides, and proteins, unless otherwise noted. As used herein, the terms “protein”, “polypeptide” and “peptide” are used interchangeably herein when referring to a gene product. The term “polypeptide”, and the terms “protein” and “peptide” which are used interchangeably herein, refers to a polymer of amino acids. Exemplary polypeptides include gene products, naturally occurring proteins, homologs, orthologs, paralogs, fragments, as well as other equivalents, variants, and analogs of the foregoing.

The terms “polypeptide fragment” or “fragment”, when used to refer to a reference polypeptide, refers to a polypeptide in which amino acid residues are deleted as compared to the reference polypeptide itself, but where the remaining amino acid sequence is usually identical to the corresponding positions in the reference polypeptide. Such deletions can occur at the amino-terminus or carboxy-terminus of the reference polypeptide, or alternatively both. Fragments typically are at least 5, 6, 8 or 10 amino acids long, at least 14 amino acids long, at least 20, 30, 40 or 50 amino acids long, at least 75 amino acids long, or at least 100, 150, 200, 300, 500 or more amino acids long. A fragment can retain one or more of the biological activities of the reference polypeptide. In certain embodiments, a fragment can comprise a druggable region, and optionally additional amino acids on one or both sides of the druggable region, which additional amino acids can number from 5, 10, 15, 20, 30, 40, 50, or up to 100 or more residues. Further, fragments can include a sub-fragment of a specific region, which sub-fragment retains a function of the region from which it is derived. In one embodiment, a fragment can have immunogenic properties.

The term “polypeptide of the invention” refers to a polypeptide comprising the amino acid sequence set forth in SEQ ID NO: 2 or SEQ ID NO: 4, or an equivalent or fragment thereof: i.e., a polypeptide comprising a sequence consisting of, or consisting essentially of, the amino acid sequence set forth in SEQ ID NO: 2 or SEQ ID NO: 4. Polypeptides of the invention include polypeptides comprising all or a portion of the amino acid sequence set forth in SEQ ID NO: 2 or SEQ ID NO: 4; the amino acid sequence set forth in SEQ ID NO: 2 or SEQ ID NO: 4 with 1 to about 2, 3, 5, 7, 10, 15, 20, 30, 50, 75 or more conservative amino acid substitutions; an amino acid sequence that is at least 60%, 70%, 80%, 90%, 95%, 96%, 97%, 98%, or 99% identical to SEQ ID NO: 2 or SEQ ID NO: 4; and functional fragments thereof. Polypeptides of the invention also include homologs, i.e., orthologs and paralogs, of SEQ ID NO: 2 or SEQ ID NO: 4.

As used herein, the term “primer” refers to a nucleic acid comprising in one embodiment 2 or more deoxyribonucleotides or ribonucleotides, in another embodiment more than 3, in another embodiment more than 8, and in yet another embodiment at least about 20 nucleotides of an exonic or intronic region. In one embodiment, an oligonucleotide is between 10 and 30 bases in length.

The term “purified” refers to an object species that is the predominant species present (i.e., on a molar basis it is more abundant than any other individual species in the composition). A “purified fraction” is a composition wherein the object species comprises at least about 50 percent (on a molar basis) of all species present. In making the determination of the purity of a species in solution or dispersion, the solvent or matrix in which the species is dissolved or dispersed is usually not included in such determination; instead, only the species (including the one of interest) dissolved or dispersed are taken into account. Generally, a purified composition will have one species that comprises more than about 80 percent of all species present in the composition, more than about 85%, 90%, 95%, 99% or more of all species present. The object species can be purified to essential homogeneity (contaminant species cannot be detected in the composition by conventional detection methods) wherein the composition consists essentially of a single species. A skilled artisan can purify a polypeptide of the invention using standard techniques for protein purification in light of the teachings herein. Purity of a polypeptide can be determined by a number of methods known to those of skill in the art, including for example, amino-terminal amino acid sequence analysis, gel electrophoresis, mass-spectrometry analysis and the methods described herein.

The terms “recombinant protein” and “recombinant polypeptide” refer to a polypeptide that is produced by recombinant DNA techniques. An example of such techniques includes when DNA encoding a polypeptide is inserted into a suitable expression vector that is in turn used to transform a host cell to produce the polypeptide encoded by the DNA.

A “reference sequence” is a defined sequence used as a basis for a sequence comparison. A reference sequence can be a subset of a larger sequence, for example, as a segment of a full-length protein given in a sequence listing such as SEQ ID NO: 2 or SEQ ID NO: 4, or can comprise a complete protein sequence. Generally, a reference sequence is at least 200, 300 or 400 nucleotides in length, frequently at least 600 nucleotides in length, and often at least 800 nucleotides in length (or the protein equivalent if it is shorter or longer in length). Because two proteins can each (1) comprise a sequence (i.e., a portion of the complete protein sequence) that is similar between the two proteins, and (2) can further comprise a sequence that is divergent between the two proteins, sequence comparisons between two (or more) proteins are typically performed by comparing sequences of the two proteins over a “comparison window” to identify and compare local regions of sequence similarity.

A “comparison window,” as used herein, refers to a conceptual segment of at least 20 contiguous amino acid positions wherein a protein sequence can be compared to a reference sequence of at least 20 contiguous amino acids and wherein the portion of the protein sequence in the comparison window can comprise additions or deletions (i.e., gaps) of 20 percent or less as compared to the reference sequence (which does not comprise additions or deletions) for optimal alignment of the two sequences. Optimal alignment of sequences for aligning a comparison window can be conducted by the local homology algorithm of Smith & Waterman, 1981, by the homology alignment algorithm of Needleman & Wunsch, 1970, by the search for similarity method of Pearson & Lipman, 1988, by computerized implementations of these algorithms (GAP, BESTFIT, FASTA, and TFASTA in the Wisconsin Genetics Software Package, available from Accelrys, Inc., San Diego, Calif., United States of America), or by inspection, and the best alignment (i.e., resulting in the highest percentage of homology over the comparison window) generated by the various methods can be identified.

The term “regulatory sequence” is a generic term used throughout the specification to refer to polynucleotide sequences, such as initiation signals, enhancers, regulators and promoters, that are necessary or desirable to affect the expression of coding and non-coding sequences to which they are operably linked. Exemplary regulatory sequences are described in Goeddel, 1990, and include, for example, the early and late promoters of SV40, adenovirus or cytomegalovirus immediate early promoter, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, i.e., Pho5, the promoters of the yeast α-mating factors, the polyhedron promoter of the baculovirus system and other sequences known to control the expression of genes of prokaryotic or eukaryotic cells or their viruses, and various combinations thereof. The nature and use of such control sequences can differ depending upon the host organism. In prokaryotes, such regulatory sequences generally include promoter, ribosomal binding site, and transcription termination sequences. The term “regulatory sequence” is intended to include, at a minimum, components whose presence can influence expression, and can also include additional components whose presence is advantageous, for example, leader sequences and fusion partner sequences. In certain embodiments, transcription of a polynucleotide sequence is under the control of a promoter sequence (or other regulatory sequence) that controls the expression of the polynucleotide in a cell-type in which expression is intended. It will also be understood that the polynucleotide can be under the control of regulatory sequences that are the same or different from those sequences which control expression of the naturally occurring form of the polynucleotide.

The term “reporter gene” refers to a nucleic acid comprising a nucleotide sequence encoding a protein that is readily detectable either by its presence or activity, including, but not limited to, luciferase, fluorescent protein (i.e., green fluorescent protein), chloramphenicol acetyl transferase, β-galactosidase, secreted placental alkaline phosphatase, β-lactamase, human growth hormone, and other secreted enzyme reporters. Generally, a reporter gene encodes a polypeptide not otherwise produced by the host cell, which is detectable by analysis of the cell(s), i.e., by the direct fluorometric, radioisotopic or spectrophotometric analysis of the cell(s) and preferably without the need to kill the cells for signal analysis. In certain instances, a reporter gene encodes an enzyme, which produces a change in fluorometric properties of the host cell, which is detectable by qualitative, quantitative, or semiquantitative function or transcriptional activation. Exemplary enzymes include esterases, β-lactamase, phosphatases, peroxidases, proteases (tissue plasminogen activator or urokinase) and other enzymes whose function can be detected by appropriate chromogenic or fluorogenic substrates known to those skilled in the art or developed in the future.

The term “sequence homology” refers to the proportion of base matches between two nucleic acid sequences or the proportion of amino acid matches between two amino acid sequences. When sequence homology is expressed as a percentage, i.e., 50%, the percentage denotes the proportion of matches over the length of sequence from a desired sequence (i.e., SEQ. ID NO: 1) that is compared to some other sequence. Gaps (in either of the two sequences) are permitted to maximize matching; gap lengths of 15 bases or less are usually used, 6 bases or less are used more frequently, with 2 bases or less used even more frequently. The term “sequence identity” means that sequences are identical (i.e., on a nucleotide-by-nucleotide basis for nucleic acids or amino acid-by-amino acid basis for polypeptides) over a window of comparison. The term “percentage of sequence identity” is calculated by comparing two optimally aligned sequences over the comparison window, determining the number of positions at which the identical amino acids occurs in both sequences to yield the number of matched positions, dividing the number of matched positions by the total number of positions in the comparison window, and multiplying the result by 100 to yield the percentage of sequence identity. Methods to calculate sequence identity are known to those of skill in the art and described in further detail herein.

As used herein, the term “sequencing” refers to determining the ordered linear sequence of nucleotides or amino acids of a DNA, RNA, or protein target sample, using conventional manual or automated laboratory techniques.

The term “small molecule” refers to a compound, which has a molecular weight of less than about 5 kilodalton (kD), less than about 2.5 kD, less than about 1.5 kD, or less than about 0.9 kD. Small molecules can be, for example, nucleic acids, peptides, polypeptides, peptide nucleic acids, peptidomimetics, carbohydrates, lipids, or other organic (carbon containing) or inorganic molecules. The term “small organic molecule” refers to a small molecule that is often identified as being an organic or medicinal compound, and does not include molecules that are exclusively nucleic acids, peptides, or polypeptides.

The term “soluble” as used herein with reference to a polypeptide of the invention or other protein means that upon expression in cell culture, at least some portion of the polypeptide or protein expressed remains in the cytoplasmic fraction of the cell and does not fractionate with the cellular debris upon lysis and centrifugation of the lysate. Solubility of a polypeptide can be increased by a variety of art recognized methods, including fusion to a heterologous amino acid sequence, deletion of amino acid residues, amino acid substitution (i.e., enriching the sequence with amino acid residues having hydrophilic side chains), and chemical modification (i.e., addition of hydrophilic groups). The solubility of polypeptides can be measured using a variety of art recognized techniques, including dynamic light scattering to determine aggregation state, UV absorption, centrifugation to separate aggregated from non-aggregated material, and SDS gel electrophoresis (i.e., the amount of protein in the soluble fraction is compared to the amount of protein in the soluble and insoluble fractions combined). When expressed in a host cell, the polypeptides of the invention can be at least about 1%, 2%, 5%, 10%, 20%, 30%, 40%, 50%, 60%, 70%, 80%, 90% or more soluble, i.e., at least about 1%, 2%, 5%, 10%, 20%, 30%, 40%, 50%, 60%, 70%, 80%, 90% or more of the total amount of protein expressed in the cell is found in the cytoplasmic fraction. In certain embodiments, a one liter culture of cells expressing a polypeptide of the invention will produce at least about 0.1, 0.2, 0.5, 1, 2, 5, 10, 20, 30, 40, 50 milligrams or more of soluble protein. In an exemplary embodiment, a polypeptide of the invention is at least about 10% soluble and will produce at least about 1 milligram of protein from a one liter cell culture.

As used herein, the term “space group” refers to the arrangement of symmetry elements of a crystal.

The term “specifically hybridizes” refers to detectable and specific nucleic acid binding. Polynucleotides, oligonucleotides, and nucleic acids of the invention selectively hybridize to nucleic acid strands under hybridization and wash conditions that minimize appreciable amounts of detectable binding to nonspecific nucleic acids. Stringent conditions can be used to achieve selective hybridization conditions as known in the art and discussed herein. Generally, the nucleic acid sequence homology between the polynucleotides, oligonucleotides, and nucleic acids of the invention and a nucleic acid sequence of interest will be at least 30%, 40%, 50%, 60%, 70%, 80%, 85%, 90%, 95%, 98%, 99%, or more. In certain instances, hybridization and washing conditions are performed under stringent conditions according to conventional hybridization procedures and as described further herein.

As used herein, the terms “structure coordinates”, “atomic coordinates”, and “structural coordinates” are used interchangeably and refer to coordinates derived from mathematical equations related to the patterns obtained on diffraction of a monochromatic beam of X-rays by the atoms (scattering centers) of a molecule in crystal form. The diffraction data are used to calculate an electron density map of the repeating unit of the crystal. The electron density maps are used to establish the positions of the individual atoms within the unit cell of the crystal.

Those of skill in the art understand that a set of coordinates determined by X-ray crystallography is not without experimental error. In general, the error in the coordinates tends to be reduced as the resolution is increased, since more experimental diffraction data is available for the model fitting and refinement. Thus, for example, more diffraction data can be collected from a crystal that diffracts to a resolution of 2.0 angstroms than from a crystal that diffracts to a lower resolution, such as 2.5 or 3.0 angstroms. Consequently, the refined structural coordinates will usually be more accurate when fitted and refined using data from a crystal that diffracts to higher resolution. The design of ligands for a CAR polypeptide depends on the accuracy of the structural coordinates. If the coordinates are not sufficiently accurate, then the design process will be ineffective. In most cases, it is very difficult or impossible to collect sufficient diffraction data to define atomic coordinates precisely when the crystals diffract to a resolution of 3.0 angstroms or poorer. Thus, in most cases, it is difficult to use X-ray structures in structure-based ligand design when the X-ray structures are based on crystals that diffract to a resolution of only 3.0 angstroms or poorer. However, common experience has shown that crystals diffracting to 2.0-2.5 angstroms or better can yield X-ray structures with sufficient accuracy to greatly facilitate structure-based drug design. Further improvement in the resolution can further facilitate structure-based design, but the coordinates obtained at 2.0-2.5 angstroms resolution are generally considered adequate for most purposes.

Also, those of skill in the art will understand that nuclear receptors can adopt different conformations when different ligands are bound, or in the absence of any ligand. In particular, in most nuclear receptors, the AF2 helix can adopt different conformations when agonists and antagonists (or inverse agonists) are bound. More subtle conformational changes occur in other parts of the LBD when the AF2 helix is shifted. Generally, structure-based design of ligands that modulate CAR activity requires an understanding of the “activated” conformation that occurs when agonists are bound (or in the absence of ligand), as well as the “repressed” conformation that occurs when antagonists (or inverse agonists) are bound. The crystal structure of CAR bound to Compound 1 provides the “repressed” structure of CAR. In one embodiment, the “activated” conformation of CAR can be modeled approximately by using the “repressed” CAR structure as a starting structure, and then adjusting the conformation of the residues at the C-terminal end of the structure, residues 332-348, to form an AF2 helix with conformation, position, and orientation similar to that observed in the “activated” conformations of other nuclear receptors. It should be noted that the X-ray structure of CAR bound to Compound 1, which is an inverse agonist, revealed a completely novel, unexpected conformation for the residues that normally comprise the AF2 helix and the AF2 linking segment. No conventional modeling procedure could have predicted this novel “repressed” structure from an X-ray structure of the “activated” conformation of CAR.

The terms “stringent conditions” or “stringent hybridization conditions” refer to conditions that promote specific hybridization between two complementary polynucleotide strands so as to form a duplex. Stringent conditions can be selected to be about 5° C. lower than the thermal melting point (Tm) for a given polynucleotide duplex at a defined ionic strength and pH. The length of the complementary polynucleotide strands and their GC content will determine the Tm of the duplex, and thus the hybridization conditions necessary for obtaining a desired specificity of hybridization. The Tm is the temperature (under defined ionic strength and pH) at which 50% of a polynucleotide sequence hybridizes to a perfectly matched complementary strand. In certain cases it can be desirable to increase the stringency of the hybridization conditions to be about equal to the Tm for a particular duplex.

A variety of techniques for estimating the Tm are available. Typically, G-C base pairs in a duplex are estimated to contribute about 3° C. to the Tm, while A-T base pairs are estimated to contribute about 2° C., up to a theoretical maximum of about 80-100° C. However, more sophisticated models of Tm are available in which G-C stacking interactions, solvent effects, the desired assay temperature and the like are taken into account. For example, probes can be designed to have a dissociation temperature (Td) of approximately 60° C., using the formula: Td=(((((3×#GC)+(2×#AT))×37)−562)/#bp)−5; where #GC, #AT, and #bp are the number of guanine-cytosine base pairs, the number of adenine-thymine base pairs, and the number of total base pairs, respectively, involved in the formation of the duplex.

Hybridization can be carried out in 5×SSC, 4×SSC, 3×SSC, 2×SSC, 1×SSC or 0.2×SSC for at least about 1 hour, 2 hours, 5 hours, 12 hours, or 24 hours. The temperature of the hybridization can be increased to adjust the stringency of the reaction, for example, from about 25° C. (room temperature), to about 45° C., 50° C., 55° C., 60° C., or 65° C. The hybridization reaction can also include another agent affecting the stringency; for example, hybridization conducted in the presence of 50% formamide increases the stringency of hybridization at a defined temperature.

The hybridization reaction can be followed by a single wash step, or two or more wash steps, which can be at the same or a different salinity and temperature. For example, the temperature of the wash can be increased to adjust the stringency from about 25° C. (room temperature), to about 45° C., 50° C., 55° C., 60° C., 65° C., or higher. The wash step can be conducted in the presence of a detergent, i.e., 0.1 or 0.2% SDS. For example, hybridization can be followed by two wash steps at 65° C. each for about 20 minutes in 2×SSC, 0.1% SDS, and optionally two additional wash steps at 65° C. each for about 20 minutes in 0.2×SSC, 0.1% SDS.

Exemplary stringent hybridization conditions include overnight hybridization at 65° C. in a solution comprising, or consisting of, 50% formamide, 10× Denhardt's Solution (0.2% Ficoll, 0.2% Polyvinylpyrrolidone, 0.2% bovine serum albumin) and 200 μg/ml of denatured carrier DNA, i.e., sheared salmon sperm DNA, followed by two wash steps at 65° C. each for about 20 minutes in 2×SSC, 0.1% SDS, and two wash steps at 65° C. each for about 20 minutes in 0.2×SSC, 0.1% SDS.

Hybridization can include hybridizing two nucleic acids in solution, or a nucleic acid in solution to a nucleic acid attached to a solid support, i.e., a filter. When one nucleic acid is on a solid support, a prehybridization step can be conducted prior to hybridization. Prehybridization can be carried out for at least about 1 hour, 3 hours or 10 hours in the same solution and at the same temperature as the hybridization solution (without the complementary polynucleotide strand).

Appropriate stringency conditions are known to those skilled in the art or can be determined experimentally by the skilled artisan. See e.g. Ausubel et al., 1994; Sambrook & Russell, 2001; Agrawal, 1993; Tibanyenda et al., 1984; Ebel et al., 1992.

The term “structural motif”, when used in reference to a polypeptide, refers to a polypeptide that, although it can have different amino acid sequences, can result in a similar structure, wherein by structure is meant that the motif forms generally the same tertiary structure, or that certain amino acid residues within the motif, or alternatively their backbone or side chains (which can or can not include the Cα atoms of the side chains) are positioned in a like relationship with respect to one another in the motif.

As applied to proteins, the term “substantial identity” means that two protein sequences, when optimally aligned, such as by the programs GAP or BESTFIT using default gap weights, typically share at least about 70 percent sequence identity, alternatively at least about 80, 85, 90, 95 percent sequence identity or more. In certain instances, residue positions that are not identical differ by conservative amino acid substitutions, which are described above.

As used herein, the term “substantially pure” refers to a polynucleotide or polypeptide that is substantially free of the sequences and molecules with which it is associated in its natural state, as well as from those molecules used in the isolation procedure. The term “substantially free” refers to that the sample is in one embodiment at least 50%, in another embodiment at least 70%, in another embodiment at least 80%, and in still another embodiment at least 90% free of the sequences and molecules with which is it associated in nature.

As used herein, the term “target cell” refers to a cell, into which it is desired to insert a nucleic acid sequence or polypeptide, or to otherwise effect a modification from conditions known to be present in the unmodified cell. A nucleic acid sequence introduced into a target cell can be of variable length. Additionally, a nucleic acid sequence can enter a target cell as a component of a plasmid or other vector or as a naked sequence.

The term “test compound” refers to a molecule to be tested by one or more screening method(s) as a putative modulator of a polypeptide of the invention or other biological entity or process. A test compound is usually not known to bind to a target of interest. The term “control test compound” refers to a compound known to bind to the target (i.e., a known agonist, antagonist, partial agonist or inverse agonist). The term “test compound” does not include a chemical added as a control condition that alters the function of the target to determine signal specificity in an assay. Such control chemicals or conditions include chemicals that 1) nonspecifically or substantially disrupt protein structure (i.e., denaturing agents (i.e., urea or guanidinium), chaotropic agents, sulfhydryl reagents (i.e., dithiothreitol and β-mercaptoethanol), and proteases), 2) generally inhibit cell metabolism (i.e., mitochondrial uncouplers) and 3) non-specifically disrupt electrostatic or hydrophobic interactions of a protein (i.e., high salt concentrations, or detergents at concentrations sufficient to non-specifically disrupt hydrophobic interactions). Further, the term “test compound” also does not include compounds known to be unsuitable for a therapeutic use for a particular indication due to toxicity of the subject. In certain embodiments, various predetermined concentrations of test compounds are used for screening such as 0.01 μM, 0.1 μM, 1.0 μM, and 10.0 μM. Examples of test compounds include, but are not limited to peptides, nucleic acids, carbohydrates, and small molecules. The term “novel test compound” refers to a test compound that is not in existence as of the filing date of this application. In certain assays using novel test compounds, the novel test compounds comprise at least about 50%, 75%, 85%, 90%, 95% or more of the test compounds used in the assay or in any particular trial of the assay.

The term “therapeutically effective amount” refers to that amount of a modulator, drug, or other molecule that is sufficient to effect treatment when administered to a subject in need of such treatment. The therapeutically effective amount will vary depending upon the subject and disease condition being treated, the weight and age of the subject, the severity of the disease condition, the manner of administration and the like, which can readily be determined by one of ordinary skill in the art.

The term “transfection” means the introduction of a nucleic acid, i.e., an expression vector, into a recipient cell, which in certain instances involves nucleic acid-mediated gene transfer. The term “transformation” refers to a process in which a cell's genotype is changed as a result of the cellular uptake of exogenous nucleic acid. For example, a transformed cell can express a recombinant form of a polypeptide of the invention or antisense expression can occur from the transferred gene so that the expression of a naturally occurring form of the gene is disrupted.

The term “transgene” means a nucleic acid sequence, which is partly or entirely heterologous to a transgenic animal or cell into which it is introduced, or, is homologous to an endogenous gene of the transgenic animal or cell into which it is introduced, but which is designed to be inserted, or is inserted, into the animal's genome in such a way as to alter the genome of the cell into which it is inserted (i.e., it is inserted at a location which differs from that of the natural gene or its insertion results in a knockout). A transgene can include one or more regulatory sequences and any other nucleic acids, such as introns, that can be necessary for optimal expression.

The term “transgenic animal” refers to any animal, for example, a mouse, rat or other non-human mammal, a bird or an amphibian, in which one or more of the cells of the animal contain heterologous nucleic acid introduced by way of human intervention, such as by transgenic techniques well known in the art. The nucleic acid is introduced into the cell, directly or indirectly, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule can be integrated within a chromosome, or it can be extrachromosomally replicating DNA. In the typical transgenic animals described herein, the transgene causes cells to express a recombinant form of a protein. However, transgenic animals in which the recombinant gene is silent are also contemplated.

As used herein, the term “unit cell” refers to a basic parallelepiped shaped block. Each unit cell comprises a complete representation of the unit of pattern, the repetition of which builds up the crystal. Thus, the term “unit cell” refers to the fundamental portion of a crystal structure that is repeated infinitely by translation in three dimensions. A unit cell is characterized by three vectors, a, b, and c, not located in one plane, which form the edges of a parallelepiped. Angles α, β and γ define the angles between the vectors: angle α is the angle between vectors b and c; angle β is the angle between vectors a and c; and angle γ is the angle between vectors a and b. The entire volume of a crystal can be constructed by regular assembly of unit cells, each unit cell comprising a complete representation of the unit of pattern, the repetition of which builds up the crystal.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the present invention.

II. Description of Tables

Table 1 is a table summarizing the crystal and data statistics obtained from the crystallized ligand-binding domain of CAR in complex with the ligand Compound 1. Data on the unit cell are presented, including data on the crystal space group, unit cell dimensions, molecules per asymmetric cell and crystal resolution.

Table 2 is a table of the atomic coordinate data obtained from X-ray diffraction from the ligand-binding domain of CAR in complex with the ligand Compound 1.

Table 3 is a table of the atomic structure coordinate data of the poly-alanine model of the conserved vitamin D receptor ligand-binding domain.

III. General Considerations

The present invention is applicable mutatis mutandis to all CARs, as discussed herein, based in part on the patterns of CAR structure and modulation that have emerged as a consequence of determining the three dimensional structure of CAR with bound ligand. Analysis and alignment of amino acid sequences, and X-ray and NMR structure determinations, have shown that nuclear receptors have a modular architecture with three main domains:

1) a variable amino-terminal domain;

2) a highly conserved DNA-binding domain (DBD); and

3) a less conserved carboxy-terminal ligand-binding domain (LBD).

In addition, nuclear receptors can have linker segments of variable length between these major domains. Sequence analysis and X-ray crystallography, including the work of the present invention, have confirmed that CARs, and indeed many NRs, also have the same general modular architecture, with the same three domains. The function of the CARs in human cells presumably requires all three domains in a single amino acid sequence. However, the modularity of the CARs permits different domains of each protein to separately accomplish certain functions.

Previous analysis of the nuclear receptors has revealed multiple discrete functional modules within the family that display generalized functional characteristics (for review see Beato et al., 1995; Kastner et al., 1995; Mangelsdorf & Evans, 1995; Tzukerman et al., 1994). A variable amino-terminal domain (A/B) is present that sometimes contains a strong and autonomous activation function (AF1), shown to be critical for cell and target gene specificity (Tora et al., 1988). A more carboxyl-terminal central region contains a DNA binding domain (DBD) characterized by two C4-type zinc fingers. The DBD binds to specific genomic response elements and thereby regulates the transcriptional activity of select genes containing the response elements. At the distal carboxyl terminus, a ligand-binding domain (LBD) is present containing a highly conserved second transactivation function (AF2) that is important for hormone-dependent transcriptional transactivation (Lanz & Rusconi, 1994).

Typically, the LBD forms a three-layered anti-parallel helical sandwich composed of 10-14 α helices and a β-sheet with 24 strands. The helices pack together so as to leave a binding pocket near the middle of the bundle, capped on one side by the β-sheet, and, in the “activated” state, capped on the other side by the AF2-helix. Comparison of apo, agonist-bound, and antagonist-bound nuclear receptor structures has led to a model for ligand-inducible receptor action. In this model, the agonist (activating) ligands tend to hold the AF2 helix in a conformation where it “caps” the binding pocket. Antagonistic ligands usually shift the AF2 helix out of this “active” position. The AF2 helix can also shift into other conformations, positions, and orientations in the absence of ligand. Constitutively active receptors such as CAR should presumably utilize a similar mechanism of action, except that the AF2 helix adopts the “active” position, capping the ligand-binding pocket, even in the absence of ligand. Inverse agonists would presumably tend to shift the AF2 helix out of this “active” position, whereas superagonists would presumably tend to hold the AF2 helix more tightly in the active position. Central to the efficient ligand-induced transcriptional activation is the recruitment of co-regulator proteins—coactivators and co-repressors, which interact with the LBD and activate or repress transactivation, respectively (Moras & Gronemeyer, 1998; Weatherman et al., 1999; McKenna & O'Malley, 2000). In general, the conformational changes described above involving the AF2 helix cause changes in the affinity of the LBD for co-repressors versus coactivators. The binding of an agonist results in a dissociation of co-repressors and brings the AF2 into a context where it can interact with transcriptional coactivators. Likewise, an antagonist would be expected to disrupt the binding of coactivators.

Sequences that function in nuclear localization, receptor dimerization, and interaction with heat-shock proteins (Gronemeyer & Laudet, 1995) are also present within the nuclear receptor substructure. Through the coordinated action of these separate functional domains, nuclear receptor activation by ligand culminates in modulation of target gene expression through DNA interactions (Tsai & O'Malley, 1994) or in certain other cases through cross-talk with other cell signaling pathways (Stein & Yang, 1995; Paech et al., 1998). In short, a ligand alters nuclear receptor function by altering the conformation of the receptor and consequently the constellation of protein-protein interactions in which the receptor is engaged (Freedman, 1999).

Some of the functions of a domain within the full-length receptor are preserved when that particular domain is isolated from the remainder of the protein. Using conventional protein chemistry techniques, a modular domain can sometimes be separated from the parent protein. Using conventional molecular biology techniques, each domain can usually be separately expressed with its original function intact or, as discussed herein below, chimeras comprising two different proteins can be constructed, wherein the chimeras retain the properties of the individual functional domains of the respective nuclear receptors from which the chimeras were generated.

The LBD is the second most highly conserved domain in these 3 domains. As its name suggests, the LBD binds ligands. With many nuclear receptors binding of the ligand can induce a conformational change in the LBD that can, in turn, increase or decrease transcription of certain target genes. The LBD also participates in other functions, including dimerization and nuclear translocation.

X-ray structures have shown that most nuclear receptor LBDs adopt the same general folding pattern. This fold includes 10-12 alpha helices arranged in a bundle, together with several beta-strands, additional alpha helices and linking segments. The major alpha helices and beta-strands have been numbered differently in different publications. The present disclosure follows the numbering scheme of Nolte et al., 1998, where the major alpha-helices and beta-strands in PPARγ were designated sequentially through the amino acid sequence as H1, H2, S1, H2′, H3, H3′, H4, H5, S2, S3, S4, H6, H7, H8, H9, H10 and HAF. The alpha helix at the C-terminal end, HAF, is also called “helix-AF”, “helix-AF2” the “AF2 helix” or “helix-12”. Most, but not all, of these alpha helices and beta-strands are observed in the structure of CAR. An additional helix, designated here as “helix-X”, is observed in the structure of CAR bound to Compound 1 on the C-terminal side of H10.

As described herein, the LBD of a CAR can be expressed, crystallized, its three dimensional structure determined with a ligand bound as disclosed in the present invention, and computational methods can be used to design ligands to its LBD.

IV. Synthesis of CAR Ligands and Intermediates

IV.A. Compound 1—An Embodiment of a Synthetic CAR Ligand

In one embodiment, the present invention provides compounds of Compound 1 (Formula (A) below) and tautomeric forms, pharmaceutically acceptable salts and solvates thereof:

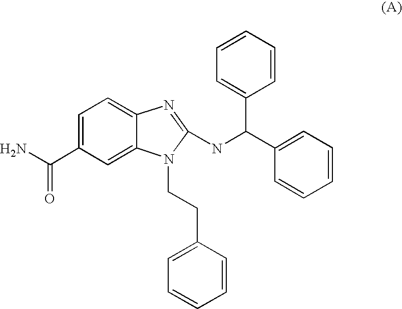

IV.B. Synthesis of Compound 1 and Intermediates

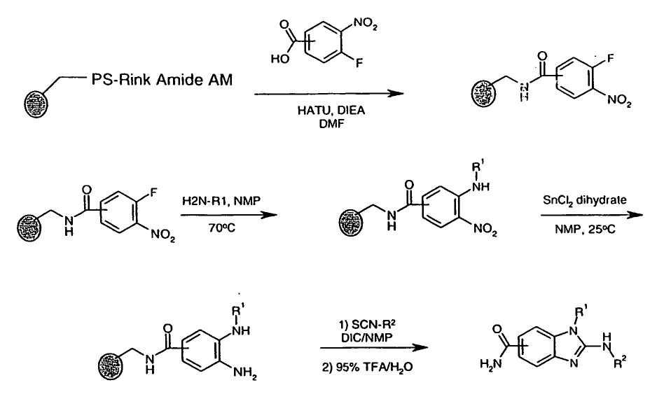

Compound 1, which was co-crystallized with the CAR LBD in the present invention, can be prepared as described in Example 6 and shown in FIG. 7. Briefly, a solution of 3-fluoro4-nitrobenzoic acid in anhydrous N,N-dimethylformamide was treated with [O-(7-azabenzotriazol-1-yl)-1,1,3,3-tetramethyluronium hexafluorophosphate] followed by N,N-diisopropylethylamine. After shaking for 5 minutes, the mixture was added to polystyrene Rink amide AM resin, and the reaction was rotated at 25° C. for 18 hours. The reaction solution was drained, and the resin was washed with N,N-dimethylformamide, dichloromethane, methanol, and dichloromethane. The dried resin was treated with a 0.5 M phenethylamine in N-methylpyrrolidinone solution and incubated with rotation for 15 hours at 70° C. The reaction was cooled to room temperature, drained, and the resin was washed as before. The resin was then treated with a 2.0 M SnCl₂.dihydrate in N-methylpyrrolidinone solution for 24 hours at 25° C. with rotation. The reaction was drained and the resin washed with 30% ethylenediamine, N,N-dimethylformamide, dichloromethane, methanol, and dichloromethane. The dried diamine resin was treated with a 0.5 M benzyhydryl isothiocyanate in N-methylpyrrolidinone solution and a 1.0 M diisopropylcarbodiimide in N-methylpyrrolidinone solution at 80° C. with rotation. After 24 hours, the reaction was cooled to 25° C., drained, and the resin was washed with N,N-dimethylformamide, dichloromethane, methanol, and dichloromethane. The resin was then treated with 95:5 TFA:H₂O and rotated at 25° C. for 3 hours. The resin was drained and washed with dichloromethane. The filtrate was concentrated in vacuo to give an oil. The oil was redissolved in dichloromethane and the solution was washed twice with saturated sodium bicarbonate. The organic layer was dried (Na₂SO₄), filtered, and concentrated in vacuo. The crude product was triturated with Et₂O/hexanes, and the solid was collected by filtration to give Compound 1 as an off-white solid.

V. Production of CAR Polypeptides

The native and mutated CAR polypeptides, and fragments thereof, of the present invention can be chemically synthesized in whole or part using techniques that are well known in the art (see e.g., Creighton, 1983, incorporated herein in its entirety). Alternatively, methods which are well known to those skilled in the art can be used to construct expression vectors containing a partial or the entire native or mutated CAR polypeptide coding sequence and appropriate transcriptional/translational control signals. These methods include in vitro recombinant DNA techniques, synthetic techniques, and in vivo recombination/genetic recombination (see e.g., the techniques described throughout Sambrook & Russell, 2001, and Ausubel et al., 1994, both incorporated herein in their entirety).

A variety of host-expression vector systems can be utilized to express a CAR coding sequence. These include but are not limited to microorganisms such as bacteria transformed with recombinant bacteriophage DNA, plasmid DNA or cosmid DNA expression vectors containing a CAR coding sequence; yeast transformed with recombinant yeast expression vectors containing a CAR coding sequence; insect cell systems infected with recombinant virus expression vectors (e.g., baculovirus) containing a CAR coding sequence; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing a CAR coding sequence; or animal cell systems. The expression elements of these systems vary in their strength and specificities.

Depending on the host/vector system utilized, any of a number of suitable transcription and translation elements, including constitutive and inducible promoters, can be used in the expression vector. For example, when cloning in bacterial systems, inducible promoters such as pL of bacteriophage X, plac, ptrp, ptac (ptrp-lac hybrid promoter) and the like can be used. When cloning in insect cell systems, promoters such as the baculovirus polyhedrin promoter can be used. When cloning in plant cell systems, promoters derived from the genome of plant cells, such as heat shock promoters; the promoter for the small subunit of ribulose bisphosphate carboxylase (RUBISCO); the promoter for the chlorophyll a/b binding protein; or from plant viruses (e.g., the 35S RNA promoter of CaMV; the coat protein promoter of TMV) can be used. When cloning in mammalian cell systems, promoters derived from the genome of mammalian cells (e.g., metallothionein promoter) or from mammalian viruses (e.g., the adenovirus late promoter; the vaccinia virus 7.5K promoter) can be used.

In each of these systems, one of ordinary skill in the art will appreciate that other promoters can be used, and as such, the list presented is not intended to be exhaustive.

VI. Analysis of Protein Properties

VI.A. Analysis of Proteins by X-ray Crystallography Generally

VI.A.1. X-ray Structure Determination

Exemplary methods for obtaining the three dimensional structure of the crystalline form of a molecule or complex are described herein and, in view of this specification, variations on these methods will be apparent to those skilled in the art (see Ducruix & Geige, 1992).

A variety of methods involving X-ray crystallography are contemplated by the present invention. For example, the present invention contemplates producing a crystallized polypeptide of the invention, or a fragment thereof, by: (a) introducing into a host cell an expression vector comprising a nucleic acid encoding for a polypeptide of the invention, or a fragment thereof; (b) culturing the host cell in a cell culture medium to express the polypeptide or fragment; (c) isolating the polypeptide or fragment from the cell culture; and (d) crystallizing the polypeptide or fragment thereof. Alternatively, the present invention contemplates determining the three dimensional structure of a crystallized polypeptide of the invention, or a fragment thereof, by: (a) crystallizing a polypeptide of the invention, or a fragment thereof, such that the crystals will diffract X-rays to a resolution of 2.5 Å or better; and (b) analyzing the polypeptide or fragment by X-ray diffraction to determine the three-dimensional structure of the crystallized polypeptide.

X-ray crystallography techniques generally require that the protein molecules be available in the form of a crystal. Crystals can be grown from a solution containing a purified polypeptide of the invention, or a fragment thereof (i.e., a ligand-binding domain), by a variety of conventional processes. These processes include, for example, batch, liquid, bridge, dialysis, and vapor diffusion (i.e., hanging drop or sitting drop methods). See e.g., McPherson, 1982; McPherson, 1990; Webe, 1991.

In certain embodiments, native crystals of the invention can be grown by adding precipitants to the concentrated solution of the polypeptide. The precipitants are added at a concentration just below that necessary to precipitate the protein. Water can be removed by controlled evaporation to produce precipitating conditions, which are maintained until crystal growth ceases.

The formation of crystals is dependent on a number of different parameters, including pH, temperature, protein concentration, the nature of the solvent and precipitant, as well as the presence of added ions or ligands to the protein. In addition, the sequence of the polypeptide being crystallized will have a significant affect on the success of obtaining crystals. Many routine crystallization experiments can be needed to screen all these parameters for the few combinations that might give crystal suitable for X-ray diffraction analysis. See e.g., Jancarik & Kim, 1991.

Crystallization robots can automate and speed up the work of reproducibly setting up large number of crystallization experiments. Once some suitable set of conditions for growing the crystal are found, variations of the condition can be systematically screened in order to find the set of conditions which allows the growth of sufficiently large, single, well ordered crystals. In certain instances, a polypeptide of the invention is co-crystallized with a ligand: in one embodiment, Compound 1.

A number of methods are available to produce suitable radiation for X-ray diffraction. For example, X-ray beams can be produced by synchrotron rings where electrons (or positrons) are accelerated through an electromagnetic field while traveling at close to the speed of light. Because the admitted wavelength can also be controlled, synchrotrons can be used as a tunable X-ray source (Hendrickson, 2000). For less conventional Laue diffraction studies, polychromatic X-rays covering a broad wavelength window are used to observe many diffraction intensities simultaneously (Stoddard, 1998). Neutrons can also be used for solving protein crystal structures (Gutberlet et al., 2001).

Before data collection commences, a protein crystal can be frozen to protect it from radiation damage. A number of different cryo-protectants can be used to assist in freezing the crystal, such as methyl pentanediol (MPD), isopropanol, ethylene glycol, glycerol, formate, citrate, mineral oil, or a low-molecular-weight polyethylene glycol (PEG). The present invention contemplates a composition comprising a polypeptide of the invention and a cryo-protectant. As an alternative to freezing the crystal, the crystal can also be used for diffraction experiments performed at temperatures above the freezing point of the solution. In these instances, the crystal can be protected from desiccation by placing it in a narrow capillary of a suitable material (generally glass or quartz) with some of the crystal growth solution included in order to maintain vapor pressure.

X-ray diffraction results can be recorded by a number of ways known to one of skill in the art. Examples of area electronic detectors include charge coupled device detectors, multi-wire area detectors, and phosphoimager detectors (Amemiya, 1997; Westbrook & Naday, 1997; Kahn & Fourme, 1997).

A suitable system for laboratory data collection might include a Bruker AXS Proteum R system, equipped with a copper rotating anode source, Confocal MAX-FLUX™ optics and a SMART 6000 charge coupled device detector. Collection of X-ray diffraction patterns is well known to those skilled in the art (see e.g. Ducruix & Geige, 1992).

The theory behind diffraction by a crystal upon exposure to X-rays is well known. Because phase information is not directly measured in the diffraction experiment and is needed to reconstruct the electron density map, methods that can recover this missing information are required. One method of solving structures ab initio is the real/reciprocal space cycling technique. Suitable real/reciprocal space cycling search programs include Shake-and-Bake (Miller et al., 1993; Weeks et al., 1994).

Other methods for deriving phases might also be needed. These techniques generally rely on the idea that if two or more measurements of the same reflection are made where strong, measurable, differences are attributable to the characteristics of a small subset of the atoms alone, then the contributions of other atoms can be, to a first approximation, ignored, and the positions of these atoms can be determined from the difference in scattering by one of the above techniques. Knowing the position and scattering characteristics of those atoms, one can calculate what phase the overall scattering must have had to produce the observed differences.

One version of this technique is the isomorphous replacement technique, which requires the introduction of new, well ordered, X-ray scatterers into the crystal. These additions are usually heavy metal atoms, (so that they make a significant difference in the diffraction pattern); and if the additions do not change the structure of the molecule or of the crystal cell, the resulting crystals should be isomorphous. Isomorphous replacement experiments are usually performed by diffusing different heavy-metal metals into the channels of a pre-existing protein crystal. Growing the crystal from protein that has been soaked in the heavy atom is also possible (Petsko, 1985). Alternatively, the heavy atom can also be reactive and attached covalently to exposed amino acid side chains (such as the sulfur atom of cysteine) or it can be associated through non-covalent interactions. It is sometimes possible to replace endogenous light metals in metallo-proteins with heavier ones, i.e., zinc by mercury, or calcium by samarium (Petsko, 1985). Exemplary sources for such heavy compounds include, but are not limited to, sodium bromide, sodium selenate, trimethyl lead acetate, mercuric chloride, methyl mercury acetate, platinum tetracyanide, platinum tetrachloride, nickel chloride, and europium chloride.

A second technique for generating differences in scattering involves the phenomenon of anomalous scattering. X-rays that cause the displacement of an electron in an inner shell to a higher shell are subsequently rescattered, but there is a time lag that shows up as a phase delay. This phase delay is observed as a (generally quite small) difference in intensity between reflections known as Friedel mates that would be identical if no anomalous scattering were present. A second effect related to this phenomenon is that differences in the intensity of scattering of a given atom will vary in a wavelength-dependent manner, giving rise to what are known as dispersive differences. In principle, anomalous scattering occurs with all atoms, but the effect is strongest with heavy atoms, and can be maximized by using X-rays at a wavelength where the energy is equal to the difference in energy between shells. The technique therefore requires the incorporation of some heavy atom much as is needed for isomorphous replacement, although for anomalous scattering a wider variety of atoms are suitable, including lighter metal atoms (copper, zinc, iron) in metallo-proteins. One method for preparing a protein for anomalous scattering involves replacing the methionine residues in whole or in part with selenium-containing seleno-methionine. Soaking with halide salts such as bromides and other non-reactive ions can also be effective (Dauter et al., 2001).

In another process, known as multiple anomalous scattering or MAD, two to four suitable wavelengths of data are collected. (Hendrickson & Ogata, 1997). Phasing by various combinations of single and multiple isomorphous and anomalous scattering are possible too. For example, SIRAS (single isomorphous replacement with anomalous scattering) utilizes both the isomorphous and anomalous differences for one derivative to derive phases. More traditionally, several different heavy atoms are soaked into different crystals to get sufficient phase information from isomorphous differences while ignoring anomalous scattering, in the technique known as multiple isomorphous replacement (MIR) (Petsko, 1985).

Additional restraints on the phases can be derived from density modification techniques. These techniques use either generally known features of electron density distribution or known facts about that particular crystal to improve the phases. For example, because protein regions of the crystal scatter more strongly than solvent regions, solvent flattening/flipping can be used to adjust phases to make solvent density a uniform flat value (Zhang et al., 1997). If more than one molecule of the protein is present in the asymmetric unit, the fact that the different molecules should be virtually identical can be exploited to further reduce phase error using non-crystallographic symmetry averaging (Villieux & Read, 1997). Suitable programs for performing these processes include DM and other programs of the CCP4 suite (Collaborative Computational Project, 1994) and CNX.

The unit cell dimensions, symmetry, vector amplitude and derived phase information can be used in a Fourier transform function to calculate the electron density in the unit cell, i.e., to generate an experimental electron density map. This can be accomplished using programs of the CNX or CCP4 packages. The resolution is measured in Ångstrom (Å) units, and is closely related to how far apart two objects need to be before they can be reliably distinguished. The smaller this number is, the higher the resolution and therefore the greater the amount of detail that can be seen. In alternative embodiments, crystals of the invention diffract X-rays to a resolution of better than about 4.0, 3.5, 3.0, 2.5, 2.0, 1.5, 1.0, 0.5 Å, or better.

As used herein, the term “modeling” includes the quantitative and qualitative analysis of molecular structure and/or function based on atomic structural information and interaction models. The term “modeling” includes conventional numeric-based molecular dynamic and energy minimization models, interactive computer graphic models, modified molecular mechanics models, distance geometry and other structure-based constraint models.

Model building can be accomplished by either the crystallographer using a computer graphics program such as TURBO or O (Jones et al., 1991) or, under suitable circumstances, by using a fully automated model building program, such as wARP (Perrakis et al., 1999) or MAID (Levitt, 2001). This structure can be used to calculate model-derived diffraction amplitudes and phases. The model-derived and experimental diffraction amplitudes can be compared and the agreement between them can be described by a parameter referred to as R-factor. A high degree of correlation in the amplitudes corresponds to a low R-factor value, with 0.0 representing exact agreement and 0.59 representing a completely random structure. Because the R-factor can be lowered by introducing more free parameters into the model, an unbiased, cross-correlated version of the R-factor known as the R-free gives a more objective measure of model quality. For the calculation of this parameter a subset of reflections (generally around 10%) are set aside at the beginning of the refinement and not used as part of the refinement target. These reflections are then compared to those predicted by the model (Kleywegt & Brunger, 1996).

The model can be improved using computer programs that maximize the probability that the observed data was produced from the predicted model, while simultaneously optimizing the model geometry. For example, the CNX program can be used for model refinement, as can the XPLOR program (Murshudov et al., 1997). In order to maximize the convergence radius of refinement, simulated annealing refinement using torsion angle dynamics can be employed in order to reduce the degrees of freedom of motion of the model (Adams et al., 1997). Where experimental phase information is available (i.e., where MAD data was collected) Hendrickson-Lattman phase probability targets can be employed. Isotropic or anisotropic domain, group or individual temperature factor refinement, can be used to model variance of the atomic position from its mean. Well-defined peaks of electron density not attributable to protein atoms are generally modeled as water molecules. Water molecules can be found by manual inspection of electron density maps, or with automatic water picking routines. Additional small molecules, including ions, cofactors, buffer molecules, or substrates can be included in the model if sufficiently unambiguous electron density is observed in a map.

In general, the R-free is rarely as low as 0.15 and can be as high as 0.35 or greater for a reasonably well-determined protein structure. The residual difference is a consequence of approximations in the model (inadequate modeling of residual structure in the solvent, modeling atoms as isotropic Gaussian spheres, assuming all molecules are identical rather than having a set of discrete conformers, etc.) and errors in the data (Lattman, 1996). In refined structures at high resolution, there are usually no major errors in the orientation of individual residues, and the estimated errors in atomic positions are usually around 0.1-0.2 up to 0.3 Å.

The three dimensional structure of a new crystal can be modeled using molecular replacement. The term “molecular replacement” refers to a method that involves generating a preliminary model of a molecule or complex whose structure coordinates are unknown, by orienting and positioning a molecule whose structure coordinates are known within the unit cell of the unknown crystal, so as best to account for the observed diffraction pattern of the unknown crystal. Phases can then be calculated from this model and combined with the observed amplitudes to give an approximate Fourier synthesis of the structure whose coordinates are unknown. This, in turn, can be subject to any of the several forms of refinement to provide a final, accurate structure of the unknown crystal (Lattman, 1985; Rossmann, 1972).

Commonly used computer software packages for molecular replacement are CNX, X-PLOR (Brunger 1992, Nature 355: 472475), AMORE (Navaza, 1994, Acta Crystallogr. A50:157-163), the CCP4 package, the MERLOT package (Fitzgerald, 1988) and XTALVIEW (McCree et al., 1992). The quality of the model can be analyzed using a program such as PROCHECK or 3D-Profiler (Laskowski et al., 1993; Luthy et al., 1992; Bowie et al., 1991).

Homology modeling (also known as comparative modeling or knowledge-based modeling) methods can also be used to develop a three dimensional model from a polypeptide sequence based on the structures of known proteins. The method utilizes a computer model of a known protein, a computer representation of the amino acid sequence of the polypeptide with an unknown structure, and standard computer representations of the structures of amino acids. This method is well known to those skilled in the art (Greer, 1985; Blundell et al., 1988; Knighton et al., 1992). Computer programs that can be used in homology modeling are QUANTA and the Homology module in the Insight II modeling package distributed by Molecular Simulations Inc. (now part of Accelrys Inc., San Diego, Calif., United States of America), or MODELLER (Rockefeller University, New York, N.Y., United States of America). These computer programs can also be used for computational loop modeling techniques. See also Tosatto et al., 2002; Fiser et al., 2000.

Once a homology model has been generated it is analyzed to determine its correctness. A computer program available to assist in this analysis is the Protein Health module in QUANTA that provides a variety of tests. Other programs that provide structure analysis along with output include PROCHECK and 3D-Profiler (Luthy et al., 1992; Bowie et al., 1991). Once any irregularities have been resolved, the entire structure can be further refined.

Other molecular modeling techniques can also be employed in accordance with this invention. See e.g., Cohen et al., 1990; Navia & Murcko, 1992.

Under suitable circumstances, the entire process of solving a crystal structure can be accomplished in an automated fashion by a system such as ELVES (http://ucxray.berkeley.edu/-jamesh/elves/index.html) with little or no user intervention.

VI.A.2. X-ray Structure

The present invention provides methods for determining some or all of the structural coordinates for amino acids of a polypeptide of the invention, or a complex thereof.

In another aspect, the present invention provides methods for identifying a druggable region of a polypeptide of the invention. For example, one such method includes: (a) obtaining crystals of a polypeptide of the invention or a fragment thereof such that the three dimensional structure of the crystallized protein can be determined to a resolution of 2.5 Å or better; (b) determining the three dimensional structure of the crystallized polypeptide or fragment using X-ray diffraction; and (c) identifying a druggable region of a polypeptide of the invention based on the three-dimensional structure of the polypeptide or fragment.

A three dimensional structure of a molecule or complex can be described by the set of atoms that best predict the observed diffraction data (that is, which possesses a minimal R value). Files can be created for the structure that defines each atom by its chemical identity, spatial coordinates in three dimensions, root mean squared deviation from the mean observed position and fractional occupancy of the observed position.

Those of skill in the art understand that a set of structure coordinates for a protein, complex, or a portion thereof, is a relative set of points that define a shape in three dimensions. Thus, it is possible that an entirely different set of coordinates could define a similar or identical shape. Moreover, slight variations in the individual coordinates can have little affect on overall shape. Such variations in coordinates can be generated because of mathematical manipulations of the structure coordinates. For example, structure coordinates could be manipulated by crystallographic permutations of the structure coordinates, fractionalization of the structure coordinates, integer additions or subtractions to sets of the structure coordinates, inversion of the structure coordinates or any combination of the above. Alternatively, modifications in the crystal structure due to mutations, additions, substitutions, and/or deletions of amino acids, or other changes in any of the components that make up the crystal, could also yield variations in structure coordinates. Such slight variations in the individual coordinates will have little affect on overall shape. If such variations are within an acceptable standard error as compared to the original coordinates, the resulting three-dimensional shape is considered to be structurally equivalent. It should be noted that slight variations in individual structure coordinates of a polypeptide of the invention or a complex thereof would not be expected to significantly alter the nature of modulators that could associate with a druggable region thereof. Thus, for example, a modulator that bound to the active site of a polypeptide of the invention would also be expected to bind to or interfere with another active site whose structure coordinates define a shape that falls within the acceptable error.

A crystal structure of the present invention can be used to make a structural or computer model of the polypeptide, complex, or portion thereof. A model can represent the secondary, tertiary, and/or quaternary structure of the polypeptide, complex, or portion. The configurations of points in space derived from structure coordinates according to the invention can be visualized as, for example, a holographic image, a stereodiagram, a model, or a computer-displayed image, and the invention thus includes such images, diagrams, or models.

VI.A.3. Structural Equivalents

Various computational analyses can be used to determine whether a molecule or the active site portion thereof is structurally equivalent with respect to its three-dimensional structure, to all or part of a structure of a polypeptide of the invention or a portion thereof.

For the purpose of this invention, any molecule or complex or portion thereof, that has a root mean square deviation of conserved residue backbone atoms (N, Cα, C, O) of less than about 1.75 Å, when superimposed on the relevant backbone atoms described by the reference structure coordinates of a polypeptide of the invention, is considered “structurally equivalent” to the reference molecule. That is to say, the crystal structures of those portions of the two molecules are substantially identical, within acceptable error. Alternatively, the root mean square deviation can be is less than about 1.50, 1.40, 1.25, 1.0, 0.75, 0.5 or 0.35 Å.

The term “root mean square deviation” is understood in the art and means the square root of the arithmetic mean of the squares of the deviations. It is a way to express the deviation or variation from a trend or object.

In another aspect, the present invention provides a scalable three-dimensional configuration of points, at least a portion of said points, and preferably all of said points, derived from structural coordinates of at least a portion of a polypeptide of the invention and having a root mean square deviation from the structure coordinates of the polypeptide of the invention of less than 1.50, 1.40, 1.25, 1.0, 0.75, 0.5 or 0.35 Å. In certain embodiments, the portion of a polypeptide of the invention is 25%, 33%, 50%, 66%, 75%, 85%, 90%, or 95% or more of the amino acid residues contained in the polypeptide.

In another aspect, the present invention provides a molecule or complex including a druggable region of a polypeptide of the invention, the druggable region being defined by a set of points having a root mean square deviation of less than about 1.75 Å from the structural coordinates for points representing (a) the backbone atoms of the amino acids contained in a druggable region of a polypeptide of the invention, (b) the side chain atoms (and optionally the Cα atoms) of the amino acids contained in such druggable region, or (c) all the atoms of the amino acids contained in such druggable region. In certain embodiments, only a portion of the amino acids of a druggable region can be included in the set of points, such as 25%, 33%, 50%, 66%, 75%, 85%, 90% or 95% or more of the amino acid residues contained in the druggable region. In certain embodiments, the root mean square deviation can be less than 1.50, 1.40, 1.25, 1.0, 0.75, 0.5, or 0.35 Å. In still other embodiments, instead of a druggable region, a stable domain, fragment, or structural motif is used in place of a druggable region.

VI.A.4. Machine Displays and Machine Readable Storage Media

The invention provides a machine-readable storage medium including a data storage material encoded with machine readable data which, when using a machine programmed with instructions for using said data, displays a graphical three-dimensional representation of any of the molecules or complexes, or portions thereof, of this invention. In another embodiment, the graphical three-dimensional representation of such molecule, complex, or portion thereof includes the root mean square deviation of certain atoms of such molecule by a specified amount, such as the backbone atoms by less than 1.5 Å. In another embodiment, a structural equivalent of such molecule, complex, or portion thereof, can be displayed. In another embodiment, the portion can include a druggable region of the polypeptide of the invention.

According to one embodiment, the invention provides a computer for determining at least a portion of the structure coordinates corresponding to X-ray diffraction data obtained from a molecule or complex, wherein said computer includes: (a) a machine-readable data storage medium comprising a data storage material encoded with machine-readable data, wherein said data comprises at least a portion of the structural coordinates of a polypeptide of the invention; (b) a machine-readable data storage medium comprising a data storage material encoded with machine-readable data, wherein said data comprises X-ray diffraction data from said molecule or complex; (c) a working memory for storing instructions for processing said machine-readable data of (a) and (b); (d) a central-processing unit coupled to said working memory and to said machine-readable data storage medium of (a) and (b) for performing a Fourier transform of the machine readable data of (a) and for processing said machine readable data of (b) into structure coordinates; and (e) a display coupled to said central-processing unit for displaying said structure coordinates of said molecule or complex. In certain embodiments, the structural coordinates displayed are structurally equivalent to the structural coordinates of a polypeptide of the invention.

In an alternative embodiment, the machine-readable data storage medium includes a data storage material encoded with a first set of machine readable data which includes the Fourier transform of the structure coordinates of a polypeptide of the invention or a portion thereof, and which, when using a machine programmed with instructions for using said data, can be combined with a second set of machine readable data including the X-ray diffraction pattern of a molecule or complex to determine at least a portion of the structure coordinates corresponding to the second set of machine readable data.

For example, a system for reading a data storage medium can include a computer including a central processing unit (CPU), a working memory which can be, i.e., random access memory (RAM) or “core” memory, mass storage memory (such as one or more disk drives or CD-ROM drives), one or more display devices (i.e., cathode-ray tube (“CRT”) displays, light emitting diode (LED) displays, liquid crystal displays (LCDs), electroluminescent displays, vacuum fluorescent displays, field emission displays (FEDs), plasma displays, projection panels, etc.), one or more user input devices (i.e., keyboards, microphones, mice, touch screens, etc.), one or more input lines, and one or more output lines, all of which are interconnected by a conventional bidirectional system bus. The system can be a stand-alone computer, or can be networked (i.e., through local area networks, wide area networks, intranets, extranets, or the internet) to other systems (i.e., computers, hosts, servers, etc.). The system can also include additional computer controlled devices such as consumer electronics and appliances.

Input hardware can be coupled to the computer by input lines and can be implemented in a variety of ways. Machine-readable data of this invention can be inputted via the use of a modem or modems connected by a telephone line or dedicated data line. Alternatively or additionally, the input hardware can include CD-ROM drives or disk drives. In conjunction with a display terminal, a keyboard can also be used as an input device.

Output hardware can be coupled to the computer by output lines and can similarly be implemented by conventional devices. By way of example, the output hardware can include a display device for displaying a graphical representation of an active site of this invention using a program such as QUANTA as described herein. Output hardware might also include a printer, so that hard copy output can be produced, or a disk drive, to store system output for later use.

In operation, a CPU coordinates the use of the various input and output devices, coordinates data accesses from mass storage devices, accesses to and from working memory, and determines the sequence of data processing steps. A number of programs can be used to process the machine-readable data of this invention. Such programs are discussed in reference to the computational methods of drug discovery as described herein. References to components of the hardware system are included as appropriate throughout the following description of the data storage medium.

Machine-readable storage devices useful in the present invention include, but are not limited to, magnetic devices, electrical devices, optical devices, and combinations thereof. Examples of such data storage devices include, but are not limited to, hard disk devices, CD devices, digital video disk devices, floppy disk devices, removable hard disk devices, magneto-optic disk devices, magnetic tape devices, flash memory devices, bubble memory devices, holographic storage devices, and any other mass storage peripheral device. It should be understood that these storage devices include necessary hardware (i.e., drives, controllers, power supplies, etc.) as well as any necessary media (i.e., disks, flash cards, etc.) to enable the storage of data.

In one embodiment, the present invention contemplates a computer readable storage medium comprising structural data, wherein the data include the identity and three-dimensional coordinates of a polypeptide of the invention or portion thereof. In another aspect, the present invention contemplates a database comprising the identity and three-dimensional coordinates of a polypeptide of the invention or a portion thereof. Alternatively, the present invention contemplates a database comprising a portion or all of the atomic coordinates of a polypeptide of the invention or portion thereof.

VI.A.5. Structurally Similar Molecules and Complexes

Structural coordinates for a polypeptide of the invention can be used to aid in obtaining structural information about another molecule or complex. This method of the invention allows determination of at least a portion of the three-dimensional structure of molecules or molecular complexes that contain one or more structural features that are similar to structural features of a polypeptide of the invention. Similar structural features can include, for example, regions of amino acid identity, conserved active site or binding site motifs, and similarly arranged secondary structural elements (i.e., a helices and 3 sheets). Many of the methods described above for determining the structure of a polypeptide of the invention can be used for this purpose as well.

For the present invention, a “structural homolog” is a polypeptide that contains one or more amino acid substitutions, deletions, additions, or rearrangements with respect to the amino acid sequence of SEQ ID NOs: 2 or 4 or other polypeptide of the invention, but that, when folded into its native conformation, exhibits or is reasonably expected to exhibit at least a portion of the tertiary (three-dimensional) structure of the polypeptide encoded by SEQ ID NOs: 2 or 4 or such other polypeptide of the invention. For example, structurally homologous molecules can contain deletions or additions of one or more contiguous or noncontiguous amino acids, such as a loop or a domain. Structurally homologous molecules also include modified polypeptide molecules that have been chemically or enzymatically derivatized at one or more constituent amino acids, including side chain modifications, backbone modifications, and N— and C-terminal modifications including acetylation, hydroxylation, methylation, amidation, and the attachment of carbohydrate or lipid moieties, cofactors, and the like.

By using molecular replacement, all or part of the structure coordinates of a polypeptide of the invention can be used to determine the structure of a crystallized molecule or complex whose structure is unknown more quickly and efficiently than attempting to determine such information ab initio. For example, in one embodiment this invention provides a method of utilizing molecular replacement to obtain structural information about a molecule or complex whose structure is unknown including: (a) crystallizing the molecule or complex of unknown structure; (b) generating an X-ray diffraction pattern from said crystallized molecule or complex; and (c) applying at least a portion of the structure coordinates for a polypeptide of the invention to the X-ray diffraction pattern to generate a three-dimensional electron density map of the molecule or complex whose structure is unknown.

In another aspect, the present invention provides a method for generating a preliminary model of a molecule or complex whose structure coordinates are unknown, by orienting and positioning the relevant portion of a polypeptide of the invention within the unit cell of the crystal of the unknown molecule or complex so as best to account for the observed X-ray diffraction pattern of the crystal of the molecule or complex whose structure is unknown.

Structural information about a portion of any crystallized molecule or complex that is sufficiently structurally similar to a portion of a polypeptide of the invention can be resolved by this method. In addition to a molecule that shares one or more structural features with a polypeptide of the invention, a molecule that has similar bioactivity, such as the same catalytic activity, substrate specificity or ligand-binding activity as a polypeptide of the invention, can also be sufficiently structurally similar to a polypeptide of the invention to permit use of the structure coordinates for a polypeptide of the invention to solve its crystal structure.

In another aspect, the method of molecular replacement is utilized to obtain structural information about a complex containing a polypeptide of the invention, such as a complex between a modulator and a polypeptide of the invention (or a domain, fragment, ortholog, homolog etc. thereof). In certain instances, the complex includes a polypeptide of the invention (or a domain, fragment, ortholog, homolog etc. thereof) co-complexed with a modulator. For example, in one embodiment, the present invention contemplates a method for making a crystallized complex comprising a polypeptide of the invention, or a fragment thereof, and a compound having a molecular weight of less than 5 kDa, the method comprising: (a) crystallizing a polypeptide of the invention such that the crystals will diffract X-rays to a resolution of 2.5 Å or better; and (b) soaking the crystal in a solution comprising the compound having a molecular weight of less than 5 kDa, thereby producing a crystallized complex comprising the polypeptide and the compound.

Using homology modeling, a computer model of a structural homolog or other polypeptide can be built or refined without crystallizing the molecule. For example, in another aspect, the present invention provides a computer-assisted method for homology modeling a structural homolog of a polypeptide of the invention including: aligning the amino acid sequence of a known or suspected structural homolog with the amino acid sequence of a polypeptide of the invention and incorporating the sequence of the homolog into a model of a polypeptide of the invention derived from atomic structure coordinates to yield a preliminary model of the homolog; subjecting the preliminary model to energy minimization to yield an energy minimized model; remodeling regions of the energy minimized model where stereochemistry restraints are violated to yield a final model of the homolog.

In another embodiment, the present invention contemplates a method for determining the crystal structure of a homolog of a polypeptide having SEQ ID NO: 2 or SEQ ID NO: 4, or equivalent thereof, the method comprising: (a) providing the three dimensional structure of a crystallized polypeptide having SEQ ID NO: 2 or SEQ ID NO: 4, or a fragment thereof; (b) obtaining crystals of a homologous polypeptide comprising an amino acid sequence that is at least 80% identical to the amino acid sequence set forth in SEQ ID NO: 2 or SEQ ID NO: 4 such that the three dimensional structure of the crystallized homologous polypeptide can be determined to a resolution of 2.5 Å or better; and (c) determining the three dimensional structure of the crystallized homologous polypeptide by X-ray crystallography based on the atomic coordinates of the three dimensional structure provided in step (a). In certain instances of the foregoing method, the atomic coordinates for the homologous polypeptide have a root mean square deviation from the backbone atoms of the polypeptide having SEQ ID NO: 2 or SEQ ID NO: 4, or a fragment thereof, of not more than 1.5 Å for all backbone atoms shared in common with the homologous polypeptide and the polypeptide having SEQ ID NO: 2 or SEQ ID NO: 4, or a fragment thereof.

In another aspect, the present invention provides a method for building a model for the activated conformation of CAR, using the repressed structure of Table 2 as a template. In one embodiment, the method comprises: (a) taking the coordinates for residues 107 to 332 directly from Table 2, effectively assuming that the conformation of this portion of CAR is similar or identical in the activated and repressed states; (b) rotating and translating an X-ray structure of VDR, the Vitamin-D receptor, so as to superimpose its core backbone atoms onto corresponding atoms from CAR; (c) combining the superimposed VDR AF2 helix, residues 416-423, with residues 107-332 from the initial CAR model of step (a), to serve as the starting model for residues 107-332 and 341-348 of the CAR protein in the activated conformation; (d) computationally mutating Val418, Leu419, Val421, Phe422 and Gly423 in the transplanted VDR AF2 helix to the corresponding amino acid types in the CAR AF2 helix, which are Leu343, Gln344, Ile346, Cys347 and Ser348, respectively; and (e) adjusting the conformations of the mutated amino acid side-chains in the AF2 helix of the CAR model, residues 343, 344, and 346-348, to avoid overlaps by using either manual manipulation within molecular graphics programs or conformational search and energy minimization. In one embodiment, the method further comprises modeling the CAR AF2 linker region, residues 333-340, by using a computational loop modeling technique, recognizing that the calculated linker conformation would probably deviate considerably from the actual linker conformation.

VII. Formation of CAR Ligand-Binding Domain-Ligand Crystals

The present invention provides crystals of CAR LBD in complex with the ligand. The crystals were obtained using the methodology disclosed in the Examples. The CAR LBD-ligand crystals, which can be native or derivative crystals, have orthorhombic unit cells (an orthorhombic unit cell is a unit cell wherein a≠b≠c, and wherein α=β=γ=90°) and space group symmetry P2₁2₁2₁. There are four CAR LBD molecules in the asymmetric unit. In this CAR crystalline form, the unit cell has dimensions of a=83.0 Å, b=116.8 Å, c=131.9 Å, and α=β=γ=90°. This crystal form can be formed in a crystallization reservoir comprising 1 μl of the protein-ligand solutions disclosed herein, and 1 μl of well buffer (e.g. 100-400 mM sodium potassium tartrate, pH 7.1-7.4).

The native and derivative co-crystals comprising a CAR. LBD and a ligand disclosed in the present invention can be obtained by a variety of techniques, including batch, liquid bridge, dialysis, vapor diffusion and hanging drop methods (see e.g., McPherson, 1982; McPherson, 1990; Weber, 1991). In one embodiment, the vapor diffusion and hanging drop methods are used for the crystallization of CAR polypeptides and fragments thereof.

Native crystals of the present invention can be grown by dissolving a substantially pure CAR polypeptide or a fragment thereof, and optionally a ligand, in an aqueous buffer containing a precipitant at a concentration just below that necessary to precipitate the protein. Water is removed by controlled evaporation to produce precipitating conditions, which are maintained until crystal growth ceases.

In one embodiment of the invention, native crystals are grown by vapor diffusion (See e.g., McPherson, 1982; McPherson, 1990). In this method, the polypeptide/precipitant solution is allowed to equilibrate in a closed container with a larger aqueous reservoir having a precipitant concentration optimal for producing crystals. Generally, less than about 25 μL of CAR polypeptide solution is mixed with an equal volume of reservoir solution, giving a precipitant concentration about half that required for crystallization. This solution is suspended as a droplet underneath a coverslip, which is sealed onto the top of the reservoir. The sealed container is allowed to stand until crystals grow. Crystals generally form within two to six weeks, and are suitable for data collection within approximately seven to ten weeks. Of course, those of skill in the art will recognize that the above-described crystallization procedures and conditions can be varied.

VIII. Solving a Crystal Structure of the Present Invention

Crystal structures of the present invention can be solved using a variety of techniques including, but not limited to isomorphous replacement, anomalous scattering, or molecular replacement methods. Computer software packages can also be used to solve a crystal structure of the present invention. Applicable software packages include, but are not limited to X-PLOR™ program (Brünger, 1992; available from Accelrys Inc, San Diego, Calif., United States of America), Xtal View (McRee, 1992; available from the San Diego Supercomputer Center, San Diego, Calif., United States of America); SHELXS 97 (Sheldrick, 1990; available from the Institute of Inorganic Chemistry, Georg-August-Universität, Gottingen, Germany); HEAVY (Terwilliger, Los Alamos National Laboratory) and SHAKE-AND-BAKE (Hauptman, 1997; Weeks et al., 1993; available from the Hauptman-Woodward Medical Research Institute, Buffalo, N.Y., United States of America). See also, Ducruix & Geige, 1992, and references cited therein.

IX. The Overall Structure of CARα in Complex With a Ligand

The structure of the LBD of CAR bound with Compound 1 has been determined to 2.15 Å. The statistics of the data and the refined structure are summarized in Table 1.

TABLE 1
Statistics of Crystallographic Data and Structure

| | CAR/with Compound 1 |
|---|---|
| Crystals | |
| Space group | P2₁2₁2₁ |
| Resolution (Å) | 40.0-2.15 |
| Unique reflections | 69,338 |
| Completeness (%) | 99.6 |
| l/σ(last shell) | 21.7 (3.1) |
| R_{sym}^{a} (%) | 9.1 |
| Refinement statistics | |
| R factor^{b} (%) | 21.5 |
| R free (%) | 25.1 |
| R.M.S.D. | 0.007 |
| bond lengths (Å) | |
| R.M.S.D. | 1.308 |
| bond angles(degrees) | |
| Total non-hydrogen atoms | 8601 |

R.M.S.D. is the root mean square deviation from ideal geometry.
^{a}R_{sym} = Σ |Iavg − Ii|/Σ Ii
^{b}R_{factor} = Σ |F_{P} − F_{Pcalc}|/Σ Fₚ, where Fₚ and F_{pcalc} are observed and calculated structure factors, R_{free} is calculated from a randomly chosen 10% of reflections that were never used in refinement and R_{factor} is calculated for the remaining 90% of reflections.

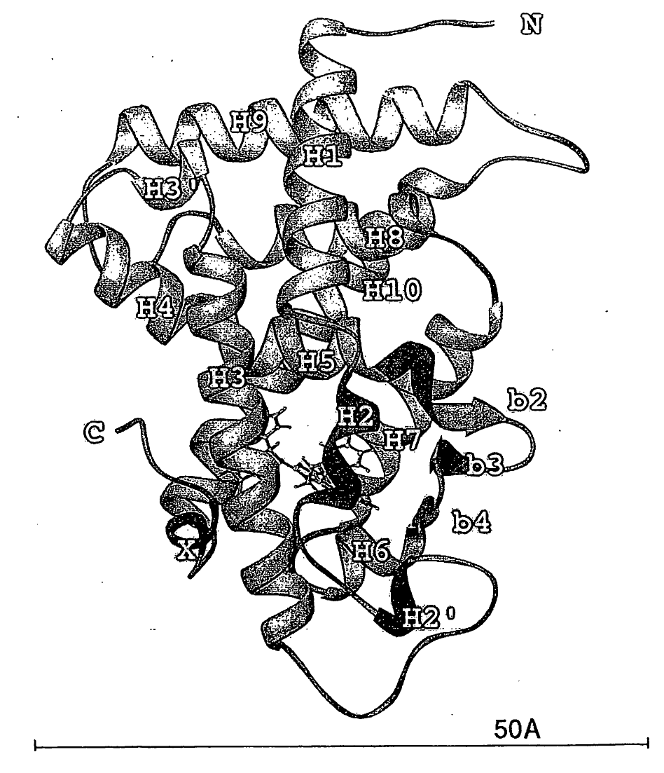

In its complex with Compound 1, an inverse agonist, the CAR LBD has a structure with approximately 11 alpha helices and a beta-sheet with 3 strands, as shown in FIG. 1. The CAR LBD amino acid sequence is more similar to PXR and VDR than to any other NR LBD sequence, with 50% identity to PXR and 40% identity to VDR in a core region corresponding to VDR residues 126-142, 227-289, 293-300, 302404 and 416-421. Slightly luwer percent identities are obtained by considering the entire LBD sequences; however, these percent identities are complicated by the presence of additional amino acids inserted between Helix-1 and Helix-3 in PXR.

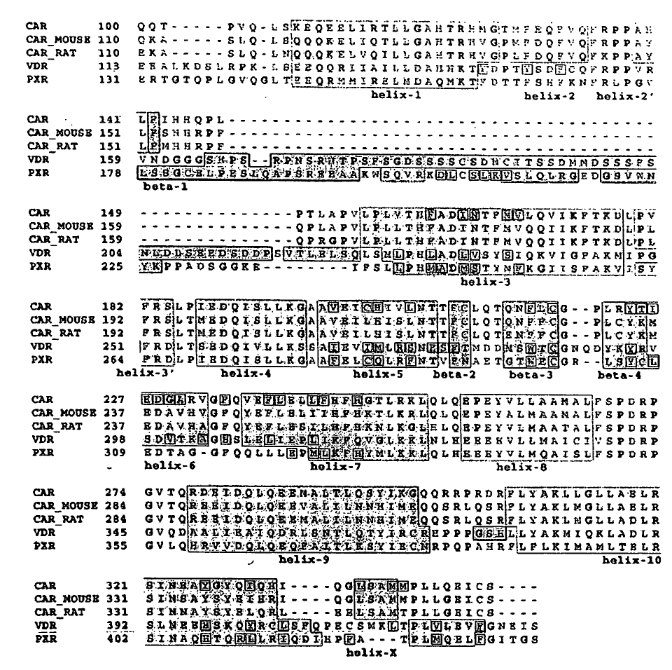

FIG. 2 gives an alignment of the human, mouse, and rat CAR sequences with the human PXR and CAR sequences, with annotation and shading to indicate structural features identified from the X-ray structures. The AF2 helix that is normally present in NR LBDs was absent in this structure, but another helix, designated here as “helix-X”, was present. Helix-X includes Leu336, Ser337, Ala338, and Met339, which lie between helix-10 and the residues that normally form the AF2 helix. The hydrogen bonding pattern in helix-X is closer to that of a 3-10 helix rather than an ideal alpha helix. The absence of the AF2 helix was initially very surprising, since the amino acid sequence at the C-terminal end of CAR is very similar to the corresponding segments in VDR and PXR (FIG. 2), where the AF2 helix has been seen in all available X-ray structures. Normally, activation of gene transcription depends on the binding of a coactivator, such as CREB binding protein (CBP) or steroid receptor coactivator-1 (SRC-1), and this in turn normally requires the presence of the AF2 helix in its active position. Thus, one would expect the AF2 helix to be present and in the active position in the unliganded, constitutively active form of CAR.

An inverse agonist such as Compound 1 or an antagonist could reduce gene transcription by shifting the AF2 helix into an alternative position, as has been observed with estrogen receptor (ER) bound to antagonists such as tamoxifen and raloxifene (Shiau et al., 1998). Alternatively, an inverse agonist or antagonist could act by unwinding the AF2 helix without necessarily moving it from its active position. Further analysis of the CAR X-ray structure suggests that helix-X interferes with the formation of the AF2 helix. Also, side-chains from Met339 and Met340, in and adjacent to helix-X, make extensive interactions with Compound 1. This suggests that Compound 1 induces the formation of helix-X, which in turn unwinds the AF2 helix, thereby preventing coactivator binding and shutting down gene transcription.

More generally, the analysis of the X-ray structure suggests that CAR exists in equilibrium with at least two major conformations. One conformation is an “activated conformation”, not yet observed by X-ray crystallography, where the AF2 helix is properly formed and resides in its active position. The second major conformation is an inactivated conformation, exemplified by the complex of CAR with Compound 1, where helix-X is present and the AF2 helix is absent. While the inventors do not wish to be bound by any particular hypothesized mechanism of action, it appears that, in the absence of ligand, CAR exists predominantly in the activated conformation. Agonist and “superagonist” compounds would tend to shift the equilibrium even farther towards this activated form, effectively increasing the fraction of the CAR receptor in the activated state to a level higher than that observed in the absence of ligand. Inverse agonists, such as Compound 1, would act by shifting the equilibrium towards the inactivated conformation, effectively decreasing the fraction of the CAR receptor in the activated state.

The structure of CAR revealed a number of other major structural differences when compared with the structures of PXR and VDR. The CAR X-ray structure allowed an accurate alignment of helix-1, confirming that PXR and VDR have 45 and 51 additional residues, respectively, in the region between helix-1 and helix-3. The conformation of this insert is unknown in VDR, as the available X-ray structures were determined with a construct where this insert was deleted. The full insert was present in the construct used for the PXR X-ray structure, and most of the insert was visible in the electron density. Surprisingly, in PXR, a segment from this insert acts to displace helix-6 from its usual position where it covers the ligand-binding pocket. This segment adopts an extended conformation that occupies less volume than helix-6, effectively opening up additional volume for the ligand in the PXR ligand-binding pocket. While the inventors do not wish to be bound by any particular hypothesized mechanism of action, based on the PXR X-ray structure and the similarity of the CAR amino acid sequence to PXR, one might expect that helix-6 would be absent or displaced away from the ligand-binding pocket, and that the ligand-binding pocket would be similarly voluminous. However, the X-ray structure of CAR reveals that helix-6 is present in CAR, and located in a position similar to that in VDR where it serves as one wall for the ligand-binding pocket. This reduces the volume available to the ligand in the ligand-binding pocket, and changes the shape of the pocket substantially. The pocket volume was calculated with the GRASP program using the atomic radii of Bondi, 1964, using a procedure where the MVP program is used to close channels to the external solvent. With this procedure, the CAR pocket has a volume of 824 Å³, similar to that of VDR, which has a volume of 871 Å³ when bound to Vitamin D, but much smaller than PXR, which has a volume of 1150-1544 Å³, depending on the ligand complexed to the protein.

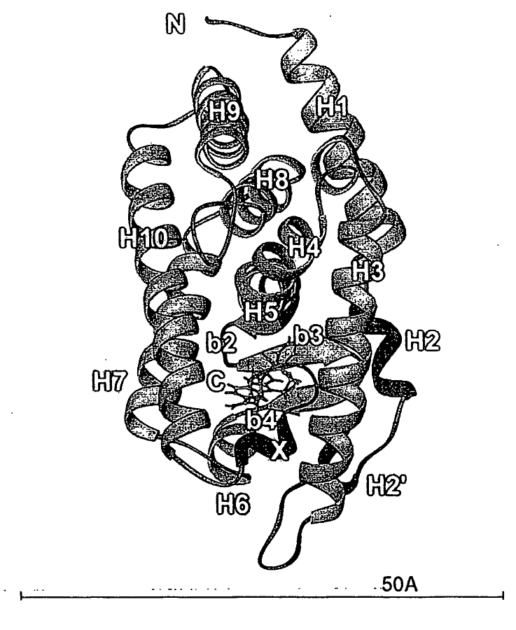

The structure of the LBD of CAR comprises 11 main alpha helices, a beta sheet with 4 strands, and additional irregular structure and shorter helices. The key features are shown in FIG. 1. Helices 3, 5, 6, 7, and 10 and beta strands 2, 3, and 4 enclose the ligand-binding pocket, like a three-layer sandwich (FIG. 6). Helix 6, which is absent or displaced in PXR, is intact in CAR, and located in a position similar to that in VDR where it serves as part of the wall of the ligand-binding site. The structure-based sequence alignment of FIG. 2 shows the secondary structures of CAR, PXR, and VDR. The presence of helix 6 in CAR reduces the size of the ligand-binding site. The limited binding pocket gives more selectivity in ligand-binding in CAR than in PXR. Binding of the antagonist in CAR causes the AF2 helix to unwind. Instead, a short sequence of amino acids located between helix 10 and the AF2 helix (Leu336, Ser337, Ala338, Met339) form a short 3-10 helix. The side chains of Leu336 and Met339, from the 3-10 helix, and Met340 form a wall that nicely fits the side of the phenyl ring of the ligand (FIGS. 1 & 3). This 3-10 helix is referred to as helix X. Steric hindrance from helix X appears to contribute to the unwinding of AF2 helix

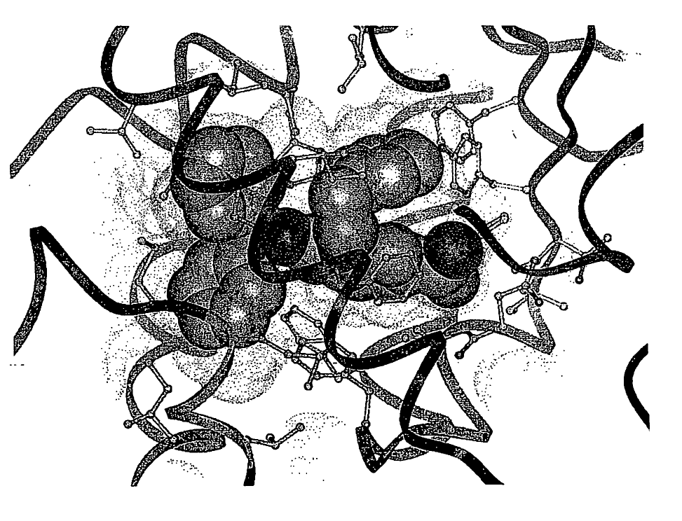

The ligand-binding site can be divided into two chambers (FIG. 5). One chamber contains the phenylethyl and benzimidazole-6-carboxamide fragments of the ligand. It is completely shielded from solvent. The other chamber contains the benzhydryl fragment of the ligand. This chamber is exposed to the solvent. The amino linker of the ligand is near the interface of the two chambers.

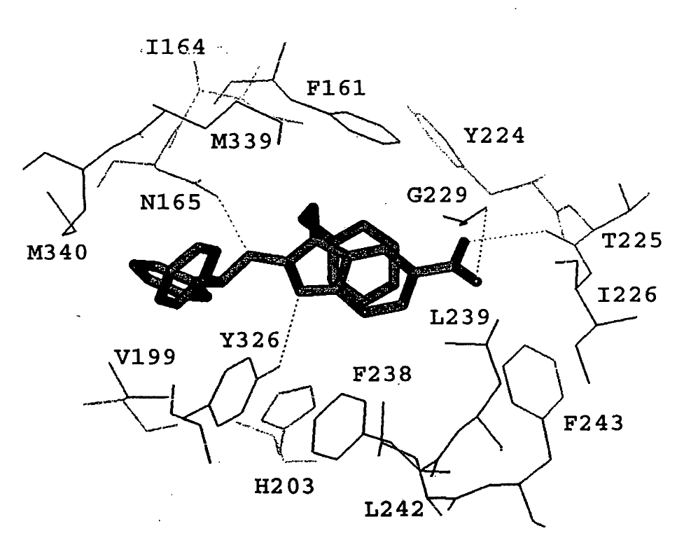

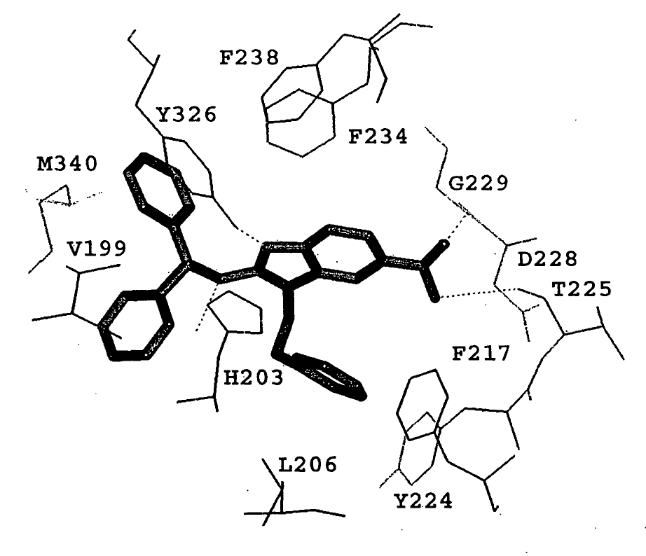

FIGS. 3 and 4 shows that the ligand fits nicely into the hydrophobic pocket of the LBD site formed mostly by aromatic or hydrophobic residues. They are Phe132, Phe161, Ile164, Asn165, Thr166, Met168, Val169, Ala198, Val199, Cys202, His203, Leu206, Phe217, Tyr224, Thr225, Ile226, Glu227, Asp228, Gly229, Ala230, Phe234, Phe238, Leu239, Leu242, Phe243, His246, Tyr326, Ile330, Leu336, Ser337, Met339, and Met340.

As shown in FIGS. 3 and 4, there are four hydrogen bonds between the ligand and LBD. The benzimidazol-6-carboxamide forms hydrogen bonds with the carbonyl oxygen of Thr225 and Gly229 amide, respectively. The unsubstituted nitrogen on the benzimidazole forms a hydrogen bond with the hydroxyl group of Tyr326. The amino group linked to the benzhydryl forms a hydrogen bond with the carboxyl oxygen of Asn165. The later two hydrogen bonds are located near the intersection of the two chambers.

X. Rational Drug Design

X.A. Generally

Modulators to polypeptides of the invention and other structurally related molecules, and complexes containing the same, can be identified and developed as set forth below and otherwise using techniques and methods known to those of skill in the art.

The present invention contemplates making any molecule that is shown to modulate the activity of a polypeptide of the invention.

In another embodiment, inhibitors, modulators of the subject polypeptides, or biological complexes containing them, can be used in the manufacture of a medicament for any number of uses, including, for example, treating any disease or other treatable condition of a patient (including humans and animals), and particularly a disease caused by aberrant CAR regulation or activity.

A number of techniques can be used to screen, identify, select, and design chemical entities capable of associating with polypeptides of the invention, structurally homologous molecules, and other molecules. Knowledge of the structure for a polypeptide of the invention, determined in accordance with the methods described herein, permits the design and/or identification of molecules and/or other modulators which have a shape complementary to the conformation of a polypeptide of the invention, or more particularly, a druggable region thereof. It is understood that such techniques and methods can use, in addition to the exact structural coordinates and other information for a polypeptide of the invention, structural equivalents thereof described above (including, for example, those structural coordinates that are derived from the structural coordinates of amino acids contained in a druggable region as described above).

The term “chemical entity”, as used herein, refers to chemical compounds, complexes of two or more chemical compounds, and fragments of such compounds or complexes. In certain instances, it is desirable to use chemical entities exhibiting a wide range of structural and functional diversity, such as compounds exhibiting different shapes (i.e., flat aromatic rings(s), puckered aliphatic rings(s), straight and branched chain aliphatics with single, double, or triple bonds) and diverse functional groups (i.e., carboxylic acids, esters, ethers, amines, aldehydes, ketones, and various heterocyclic rings).

In one aspect, the method of drug design generally includes computationally evaluating the potential of a selected chemical entity to associate with any of the molecules or complexes of the present invention (or portions thereof). For example, this method can include the steps of (a) employing computational means to perform a fitting operation between the selected chemical entity and a druggable region of the molecule or complex; and (b) analyzing the results of said fitting operation to quantify the association between the chemical entity and the druggable region.

A chemical entity can be examined either through visual inspection or through the use of computer modeling using a docking program such as GRAM, DOCK, or AUTODOCK (Dunbrack et al., 1997). This procedure can include computer fitting of chemical entities to a target to ascertain how well the shape and the chemical structure of each chemical entity will complement or interfere with the structure of the subject polypeptide (Bugg et al, 1993; West et al, 1995). Computer programs can also be employed to estimate the attraction, repulsion, and steric hindrance of the chemical entity to a druggable region, for example. Generally, the tighter the fit (i.e., the lower the steric hindrance, and/or the greater the attractive force) the more potent the chemical entity will be because these properties are consistent with a tighter binding constant. Furthermore, the more specificity in the design of a chemical entity the more likely that the chemical entity will not interfere with related proteins, which can minimize potential side-effects due to unwanted interactions.

A variety of computational methods for molecular design, in which the steric and electronic properties of druggable regions are used to guide the design of chemical entities, are known. See e.g., Cohen et al., 1990; Kuntz et al., 1982; DesJarlais, 1988; Bartlett et al., 1989; Goodford et al., 1985; DesJarlais et al., 1986. Directed methods generally fall into two categories: (1) design by analogy in which 3-D structures of known chemical entities (such as from a crystallographic database) are docked to the druggable region and scored for goodness-of-fit; and (2) de novo design, in which the chemical entity is constructed piece-wise in the druggable region. The chemical entity can be screened as part of a library or a database of molecules. Databases which can be used include ACD (MDL Systems Inc., San Leandro, Calif., United States of America), NCI (National Cancer Institute, Bethesda, Md., United States of America), CCDC (Cambridge Crystallographic Data Center, Cambridge, England, United Kingdom), CAST (Chemical Abstract Service), Derwent (Derwent Information Limited, London, England, United Kingdom), Maybridge (Maybridge Chemical Company Ltd., Cornwall, England, United Kingdom), Aldrich (Aldrich Chemical Company, St. Louis, Mo., United States of America), DOCK (University of California in San Francisco, San Francisco, Calif., United States of America), and the Directory of Natural Products (Chapman & Hall). Computer programs such as CONCORD (Tripos Inc., St. Louis, Mo., United States of America) or DB-Converter (Molecular Simulations Limited, Cambridge, England, United Kingdom) can be used to convert a data set represented in two dimensions to one represented in three dimensions.

Chemical entities can be tested for their capacity to fit spatially with a druggable region or other portion of a target protein. As used herein, the term “fits spatially” means that the three-dimensional structure of the chemical entity is accommodated geometrically by a druggable region. A favorable geometric fit occurs when the surface area of the chemical entity is in close proximity with the surface area of the druggable region without forming unfavorable interactions. A favorable complementary interaction occurs where the chemical entity interacts by hydrophobic, aromatic, ionic, dipolar, or hydrogen donating and accepting forces. Unfavorable interactions can be steric hindrance between atoms in the chemical entity and atoms in the druggable region.

If a model of the present invention is a computer model, the chemical entities can be positioned in a druggable region through computational docking. If, on the other hand, the model of the present invention is a structural model, the chemical entities can be positioned in the druggable region by, for example, manual docking. As used herein the term “docking” refers to a process of placing a chemical entity in close proximity with a druggable region, or a process of finding low energy conformations of a chemical entity/druggable region complex.

In an illustrative embodiment, the design of potential modulator begins from the general perspective of shape complimentary for the druggable region of a polypeptide of the invention, and a search algorithm is employed which is capable of scanning a database of small molecules of known three-dimensional structure for chemical entities which fit geometrically with the target druggable region. Most algorithms of this type provide a method for finding a wide assortment of chemical entities that are complementary to the shape of a druggable region of the subject polypeptide. Each of a set of chemical entities from a particular data-base, such as the Cambridge Crystallographic Data Bank (CCDB) (Allen et al., 1973), is individually docked to the druggable region of a polypeptide of the invention in a number of geometrically permissible orientations with use of a docking algorithm. In certain embodiments, a set of computer algorithms called DOCK, can be used to characterize the shape of invaginations and grooves that form the active sites and recognition surfaces of the druggable region (Kuntz et al., 1982). The program can also search a database of small molecules for templates whose shapes are complementary to particular binding sites of a polypeptide of the invention (DesJarlais et al, 1988).

The orientations are evaluated for goodness-of-fit and the best are kept for further examination using molecular mechanics programs, such as AMBER or CHARMM. Such algorithms have previously proven successful in finding a variety of chemical entities that are complementary in shape to a druggable region.

Goodford et al, 1985 and Boobbyer et al., 1989 have produced a computer program (GRID) that seeks to determine regions of high affinity for different chemical groups (termed probes) of the druggable region. GRID hence provides a tool for suggesting modifications to known chemical entities that might enhance binding. It can be anticipated that some of the sites discerned by GRID as regions of high affinity correspond to “pharmacophoric patterns” determined inferentially from a series of known ligands. As used herein, a “pharmacophoric pattern” is a geometric arrangement of features of chemical entities that is believed to be important for binding. Attempts have been made to use pharmacophoric patterns as a search screen for novel ligands (Jakes et al., 1987; Brint & Willett, 1987; Jakes et al., 1986).

Yet a further embodiment of the present invention utilizes a computer algorithm such as CLIX which searches such databases as CCDB for chemical entities which can be oriented with the druggable region in a way that is both sterically acceptable and has a high likelihood of achieving favorable chemical interactions between the chemical entity and the surrounding amino acid residues. The method is based on characterizing the region in terms of an ensemble of favorable binding positions for different chemical groups and then searching for orientations of the chemical entities that cause maximum spatial coincidence of individual candidate chemical groups with members of the ensemble. The algorithmic details of CLIX are described in Lawrence et al., 1992.

In this way, the efficiency with which a chemical entity can bind to or interfere with a druggable region can be tested and optimized by computational evaluation. For example, for a favorable association with a druggable region, a chemical entity must preferably demonstrate a relatively small difference in energy between its bound and fine states (i.e., a small deformation energy of binding). Thus, certain, more desirable chemical entities will be designed with a deformation energy of binding of not greater than about 10 kcal/mole, and more preferably, not greater than 7 kcal/mole. Chemical entities can interact with a druggable region in more than one conformation that is similar in overall binding energy. In those cases, the deformation energy of binding is taken to be the difference between the energy of the free entity and the average energy of the conformations observed when the chemical entity binds to the target.

In this way, the present invention provides computer-assisted methods for identifying or designing a potential modulator of the activity of a polypeptide of the invention including: supplying a computer modeling application with a set of structure coordinates of a molecule or complex, the molecule or complex including at least a portion of a druggable region from a polypeptide of the invention; supplying the computer modeling application with a set of structure coordinates of a chemical entity; and determining whether the chemical entity is expected to bind to the molecule or complex, wherein binding to the molecule or complex is indicative of potential modulation of the activity of a polypeptide of the invention.

In another aspect, the present invention provides a computer-assisted method for identifying or designing a potential modulator to a polypeptide of the invention, supplying a computer modeling application with a set of structure coordinates of a molecule or complex, the molecule or complex including at least a portion of a druggable region of a polypeptide of the invention; supplying the computer modeling application with a set of structure coordinates for a chemical entity; evaluating the potential binding interactions between the chemical entity and active site of the molecule or molecular complex; structurally modifying the chemical entity to yield a set of structure coordinates for a modified chemical entity, and determining whether the modified chemical entity is expected to bind to the molecule or complex, wherein binding to the molecule or complex is indicative of potential modulation of the polypeptide of the invention.

In one embodiment, a potential modulator can be obtained by screening a peptide library (Scott & Smith, 1990; Cwirla et al., 1990; Devlin et al., 1990). A potential modulator selected in this manner could then be systematically modified by computer modeling programs until one or more promising potential drugs are identified. Such analysis has been shown to be effective in the development of HIV protease inhibitors (Lam et al., 1994; Wlodawer et al., 1993; Appelt, 1993; Erickson, 1993). Alternatively a potential modulator can be selected from a library of chemicals such as those that can be licensed from third parties, such as chemical and pharmaceutical companies. A third alternative is to synthesize the potential modulator de novo.

For example, in certain embodiments, the present invention provides a method for making a potential modulator for a polypeptide of the invention, the method including synthesizing a chemical entity or a molecule containing the chemical entity to yield a potential modulator of a polypeptide of the invention, the chemical entity having been identified during a computer-assisted process including supplying a computer modeling application with a set of structure coordinates of a molecule or complex, the molecule or complex including at least one druggable region from a polypeptide of the invention; supplying the computer modeling application with a set of structure coordinates of a chemical entity; and determining whether the chemical entity is expected to bind to the molecule or complex at the active site, wherein binding to the molecule or complex is indicative of potential modulation. This method can further include the steps of evaluating the potential binding interactions between the chemical entity and the active site of the molecule or molecular complex and structurally modifying the chemical entity to yield a set of structure coordinates for a modified chemical entity, which steps can be repeated one or more times.

Once a potential modulator is identified, it can then be tested in any standard assay for the macromolecule depending of course on the macromolecule, including in high throughput assays. Further refinements to the structure of the modulator will generally be necessary and can be made by the successive iterations of any and/or all of the steps provided by the particular screening assay, in particular further structural analysis by i.e., 15N NMR relaxation rate determinations or X-ray crystallography with the modulator bound to the subject polypeptide. These studies can be performed in conjunction with biochemical assays.

Once identified, a potential modulator can be used as a model structure, and analogs to the compound can be obtained. The analogs are then screened for their ability to bind the subject polypeptide. An analog of the potential modulator might be chosen as a modulator when it binds to the subject polypeptide with a higher binding affinity than the predecessor modulator.

In a related approach, iterative drug design is used to identify modulators of a target protein. Iterative drug design is a method for optimizing associations between a protein and a modulator by determining and evaluating the three dimensional structures of successive sets of protein/modulator complexes. In iterative drug design, crystals of a series of protein/modulator complexes are obtained and then the three-dimensional structures of each complex is solved. Such an approach provides insight into the association between the proteins and modulators of each complex. For example, this approach can be accomplished by selecting modulators with inhibitory activity, obtaining crystals of this new protein/modulator complex, solving the three dimensional structure of the complex, and comparing the associations between the new protein/modulator complex and previously solved protein/modulator complexes. By observing how changes in the modulator affected the protein/modulator associations, these associations can be optimized.

In addition to designing and/or identifying a chemical entity to associate with a druggable region, as described above, the same techniques and methods can be used to design and/or identify chemical entities that either associate, or do not associate, with affinity regions, selectivity regions or undesired regions of protein targets. By such methods, selectivity for one or a few targets, or alternatively for multiple targets, from the same species or from multiple species, can be achieved.

For example, a chemical entity can be designed and/or identified for which the binding energy for one druggable region, i.e., an affinity region or selectivity region, is more favorable than that for another region, i.e., an undesired region, by about 20%, 30%, 50% to about 60% or more. It can be the case that the difference is observed between (a) more than two regions, (b) between different regions (selectivity, affinity or undesirable) from the same target, (c) between regions of different targets, (d) between regions of homologs from different species, or (e) between other combinations. Alternatively, the comparison can be made by reference to the K_{d}, usually the apparent K_{d}, of said chemical entity with the two or more regions in question.

In another aspect, prospective modulators are screened for binding to two nearby druggable regions on a target protein. For example, a modulator that binds a first region of a target polypeptide does not bind a second nearby region. Binding to the second region can be determined by monitoring changes in a different set of amide chemical shifts in either the original screen or a second screen conducted in the presence of a modulator (or potential modulator) for the first region. From an analysis of the chemical shift changes, the approximate location of a potential modulator for the second region is identified. Optimization of the second modulator for binding to the region is then carried out by screening structurally related compounds (i.e., analogs as described above).

When modulators for the first region and the second region are identified, their location and orientation in the ternary complex can be determined experimentally. On the basis of this structural information, a linked compound, i.e., a consolidated modulator, is synthesized in which the modulator for the first region and the modulator for the second region are linked. In certain embodiments, the two modulators are covalently linked to form a consolidated modulator. This consolidated modulator can be tested to determine if it has a higher binding affinity for the target than either of the two individual modulators. A consolidated modulator is selected as a modulator when it has a higher binding affinity for the target than either of the two modulators. Larger consolidated modulators can be constructed in an analogous manner, i.e., linking three modulators which bind to three nearby regions on the target to form a multilinked consolidated modulator that has an even higher affinity for the target than the linked modulator. In this example, it is assumed that is desirable to have the modulator bind to all the druggable regions. However, it can be the case that binding to certain of the druggable regions is not desirable, so that the same techniques can be used to identify modulators and consolidated modulators that show increased specificity based on binding to at least one but not all druggable regions of a target.

The present invention provides a number of methods that use drug design as described above. For example, in one aspect, the present invention contemplates a method for designing a candidate compound for screening for inhibitors of a polypeptide of the invention, the method comprising: (a) determining the three dimensional structure of a crystallized polypeptide of the invention or a fragment thereof; and (b) designing a candidate inhibitor based on the three dimensional structure of the crystallized polypeptide or fragment.

In another aspect, the present invention provides a method for identifying a potential inhibitor of a polypeptide of the invention, the method comprising: (a) providing the three-dimensional coordinates of a polypeptide of the invention or a fragment thereof; (b) identifying a druggable region of the polypeptide or fragment; and (c) selecting from a database at least one compound that comprises three dimensional coordinates which indicate that the compound can bind the druggable region; (d) wherein the selected compound is a potential inhibitor of a polypeptide of the invention.

In another aspect, the present invention contemplates a method for identifying a potential modulator of a molecule comprising a druggable region similar to that of SEQ ID NO: 2 or SEQ ID NO: 4, the method comprising: (a) using the atomic coordinates of amino acid residues from SEQ ID NO: 2 or SEQ ID NO: 4, or a fragment thereof, ± a root mean square deviation from the backbone atoms of the amino acids of not more than 1.5 Å, to generate a three-dimensional structure of a molecule comprising a druggable region that is a portion of SEQ ID NO: 2 or SEQ ID NO: 4; (b) employing the three dimensional structure to design or select the potential modulator; (c) synthesizing the modulator; and (d) contacting the modulator with the molecule to determine the ability of the modulator to interact with the molecule.

In another aspect, the present invention contemplates an apparatus for determining whether a compound is a potential inhibitor of a polypeptide having SEQ ID NO: 2 or SEQ ID NO: 4, the apparatus comprising: (a) a memory that comprises: (i) the three dimensional coordinates and identities of the atoms of a polypeptide of the invention or a fragment thereof that form a druggable site; and (ii) executable instructions; and (b) a processor that is capable of executing instructions to: (i) receive three-dimensional structural information for a candidate compound; (ii) determine if the three-dimensional structure of the candidate compound is complementary to the structure of the interior of the druggable site; and (iii) output the results of the determination.

In another aspect, the present invention contemplates a method for designing a potential compound for the prevention or treatment of a disease or disorder, the method comprising: (a) providing the three dimensional structure of a crystallized polypeptide of the invention, or a fragment thereof; (b) synthesizing a potential compound for the prevention or treatment of a disease or disorder based on the three dimensional structure of the crystallized polypeptide or fragment; (c) contacting a polypeptide of the present invention or a PDE with the potential compound; and (d) assaying the activity of a polypeptide of the present invention, wherein a change in the activity of the polypeptide indicates that the compound can be useful for prevention or treatment of a disease or disorder.

In another aspect, the present invention contemplates a method for designing a potential compound for the prevention or treatment of a disease or disorder, the method comprising: (a) providing structural information of a druggable region derived from NMR spectroscopy of a polypeptide of the invention, or a fragment thereof; (b) synthesizing a potential compound for the prevention or treatment of a disease or disorder based on the structural information; (c) contacting a polypeptide of the present invention or a PDE with the potential compound; and (d) assaying the activity of a polypeptide of the present invention, wherein a change in the activity of the polypeptide indicates that the compound can be useful for prevention or treatment of a disease or disorder.

X.B. Methods of Designing CAR LBD Ligand Compounds

As discussed above, the analysis of the CAR X-ray structure suggests that CAR can adopt at least two major conformations. One major conformation corresponds to the activated state of CAR, where helix-X is absent, and where the AF2 helix is properly formed and resides in its active position. The second major conformation corresponds to the inactivated conformation, exemplified by the complex of CAR with Compound 1, where helix-X is present and where the AF2 helix is absent. In both conformations, the ligand-binding pocket is capped by the C-terminal tail, residues 340-348. These residues adopt different conformations in the activated and inactivated states of CAR, effectively covering the pocket with a cap that can assume at least two alternative shapes. Some CAR ligands might bind preferentially to the activated conformation of CAR, whereas some other CAR ligands might bind preferentially to the inactivated conformation of CAR. There might also be some ligands that bind equally well to either conformation of CAR. When a ligand binds preferentially to a particular conformational state, it will lower the energy of that state, thereby shifting the equilibrium towards that state, and increasing the fraction of the CAR receptor that exists in that state. This thermodynamic principle can be used together with the three dimensional structure of CAR to design chemical compounds that bind to specific conformational states of CAR, thereby increasing or decreasing the level of transcription in genes regulated by CAR.

The present X-ray structure of CAR bound to Compound 1 provides an accurate three-dimensional structure of the ligand-binding pocket in the inactivated conformational state of CAR. Novel ligands can be designed to fit this specific pocket using a variety of computational methods, discussed below. Alternatively, known ligands can be docked into the ligand-binding pocket, using a variety of docking programs and algorithms. These docked structures can be examined graphically to suggest chemical modifications that would improve their fit to the pocket, or their binding to the receptor. Alternatively, known ligands can be complexed with the CAR protein and crystallized using the methods of this invention, allowing the structure of the complex to be determined by X-ray crystallography. The three dimensional structures can be examined graphically to suggest chemical modifications that would improve their fit to the pocket, or their binding to the receptor.

The present X-ray structure of CAR can also be used as a template to build a three-dimensional model of the structure of the activated form of CAR. For example, residues 107 to 332, corresponding to helix-1 through most of helix-10, are taken to have exactly the same coordinates as in the template CAR structure. The AF2 helix, CAR residues 341-348, is then built using the structure of VDR as the template. The VDR template structure is superimposed onto the CAR structure using standard methods as disclosed herein and as would be apparent to one of ordinary skill in the art after a review of the present disclosure. The AF2 helix from VDR, residues 416-423, is then removed from the VDR template and transplanted into the model for CAR, without any adjustment of its coordinates. Five of the residues in the VDR AF2 helix have amino acid types different from the corresponding residues in the CAR AF2 helix. These residues are VDR Val418, Leu419, Val421, Phe422, and Gly423, which correspond to CAR Leu343, Gln344, Ile346, Cys347, and Ser348, respectively. These five residues are computationally “mutated” in the model, to obtain the covalent structure corresponding to the desired amino acids in CAR. The C-terminal Ser348 is further modified to obtain a free carboxylate as normally occurs at the C-terminal end of a protein chain.

These computational mutations can be carried out using amino acid replacement and builder functionality in molecular graphics programs such as Insight-II, available from Accelrys, or using non-graphical molecular mechanics software such as MVP. The side-chain conformations are then adjusted using computer graphics, such as Insight-II, or other energy-based procedures, such as in MVP, to obtain a reasonable overall fit. It is more difficult to obtain a reasonable conformation for the eight residues in the AF2 linker, CAR residues 333-340. The VDR linker, residues 407-415, cannot be used as the template for the CAR linker because it has nine residues, and because its N-terminal end-point is different from that required in CAR. Likewise, the PXR linker, residues 418-422, is too short to serve as a template for the CAR linker. For structure-based drug design, a conservative approach is to omit the linker residues rather than to model the linker incorrectly. Consequently, in one embodiment the linker, residues 333-340, is omitted from the activated CAR model. This model for the activated state of CAR then provides a binding site for the ligand design processes described elsewhere herein. Specifically, various computer software programs can be used to design novel ligands that would fit the specific pocket in the model for the activated form of CAR. Docking calculations can be used to predict how known CAR activators will bind to the activated form of CAR or to identify other available compounds that might bind. These predicted complex structures can then be examined by computer graphics to suggest specific chemical modifications that would enhance the binding to the activated state of CAR.

To be useful as a therapeutic agent, a chemical compound that acts through CAR must induce the appropriate level of CAR activity in relevant tissues. In principle, this can be achieved by adjusting the CAR conformational equilibrium so that appropriate fractions of the CAR protein exist in the activated and inactivated states. This in turn can be achieved with ligands that bind almost exclusively to one or the other of the two major conformational states. The design of ligands that are selective for a specific conformational state is facilitated by consideration of how these ligands might bind to each of the two conformational states. Binding modes can be obtained using docking calculations, and then examined graphically to suggest chemical modifications that would make binding to a particular conformational state either more favorable or less favorable. Iterative application of these techniques can yield ligands with the desired level of selectivity for the particular conformational state of CAR, thereby achieving the desired level of CAR activity. Ligands that can bind to both conformational states of the CAR protein can also be designed. This is also facilitated by consideration of how the ligands might bind to each of the two conformational states, using the same approach as discussed above, but this time seeking chemical structures and chemical modifications that would permit binding to both conformational states.

The methods of this invention can also be used to suggest possible chemical modifications of a compound that might reduce or minimize its effect on CAR. This approach can be useful in drug discovery projects aiming to find compounds that modulate the activity of some other target molecule, where modulation of CAR activity is an undesirable side effect. This approach is useful in engineering CAR activity out of other, non-drug molecules. Humans and other animals are exposed to a wide range of different chemical compounds, some of which might act on CAR in an undesirable manner. Such a compound could be complexed with CAR and crystallized using the methods of the present invention. The structure could then be determined by X-ray crystallography. Alternatively, the structure of the complex could be predicted computationally using molecular docking software. In this case, compounds that tend to activate CAR would be docked into a model or structure of the activated form of CAR, whereas compounds that tend to reduce the activity of CAR would be docked into a model or structure of an inactivated form of CAR, such as its complex with Compound 1 presented here.

Whether the structure is obtained by X-ray crystallography or computational methods, the structure would be examined by computer graphics to suggest chemical modifications that would minimize the tendency to bind to CAR. For example, substituents could be introduced onto the compound that would project into volume occupied by the CAR protein. Alternatively, a region of the molecule that binds to a lipophilic region of the CAR binding site could be modified to make it more polar, thus reducing its tendency to bind to CAR. Alternatively, a polar group of the compound that makes a hydrogen bonding interaction with CAR could be identified and modified to an alternative group that fails to make the hydrogen bond. Appropriate chemical modifications can be chosen such that the desirable properties and behavior of the compound would be retained.

The design of candidate substances, also referred to as “compounds” or “candidate compounds”, that bind to or modulate nuclear receptor (NR) LBD (for example, CAR LBD)-mediated activity according to the present invention generally involves consideration of two factors. First, the compound must be capable of chemically and structurally associating with a NR LBD. Non-covalent molecular interactions important in the association of a NR LBD with its substrate include hydrogen bonding, van der Waals interactions, and hydrophobic interactions. The interaction between an atom of an LBD amino acid and an atom of an LBD ligand can be made by any force or attraction described in nature. Usually the interaction between the atom of the amino acid and the ligand will be the result of a hydrogen bonding interaction, charge interaction, hydrophobic interaction, van der Waals interaction, or dipole interaction. In the case of the hydrophobic interaction, it is recognized that this is not a per se interaction between the amino acid and ligand, but rather the usual result, in part, of the repulsion of water or other hydrophilic groups from a hydrophobic surface. Reducing or enhancing the interaction of the LBD and a ligand can be measured by calculating or testing binding energies, either computationally or using thermodynamic or kinetic methods known in the art.

Second, the compound must be able to assume a conformation that allows it to associate with a NR LBD. Although certain portions of the compound will not directly participate in this association with a NR LBD, those portions can still influence the overall conformation of the molecule. This influence on conformation, in turn, can have a significant impact on potency. Such conformational requirements include the overall three-dimensional structure and orientation of the chemical entity or compound in relation to all or a portion of the binding site, e.g., the ligand-binding pocket or an accessory binding site of a NR LBD, or the spacing between functional groups of a compound comprising several chemical entities that directly interact with a NR LBD.

Chemical modifications can enhance or reduce interactions of an atom of a LBD amino acid and an atom of an LBD ligand. Steric hindrance can be a common approach for changing the interaction of a LBD binding pocket with an activation domain. Chemical modifications are introduced in one embodiment at C—H, C—, and C—OH positions in a ligand, where the carbon is part of the ligand structure that remains the same after modification is complete. In the case of C—H, C could have 1, 2, or 3 hydrogens, but usually only one hydrogen will be replaced. The H or OH can be removed after modification is complete and replaced with a desired chemical moiety.

The potential binding effect of a chemical compound on a NR LBD can be analyzed prior to its actual synthesis and testing by the use of computer modeling techniques that employ the coordinates of a crystalline NR LBD, for example a CAR LBD polypeptide of the present invention. If the theoretical structure of the given compound suggests insufficient interaction and association between it and a NR LBD, synthesis and testing of the compound is obviated. However, if computer modeling indicates a strong interaction, the molecule can then be synthesized and tested for its ability to bind and modulate the activity of a NR LBD. In this manner, synthesis of unproductive or inactive compounds can be avoided.

A binding compound of a NR LBD polypeptide (in one embodiment a CAR LBD) can be computationally evaluated and designed via a series of steps in which chemical entities or fragments are screened and selected for their ability to associate with an individual binding site or other area of a crystalline CAR LBD polypeptide of the present invention and to interact with the amino acids disposed in the binding sites.

Interacting amino acids forming contacts with a ligand and the atoms of the interacting amino acids are usually 2 to 4 angstroms away from the center of the atoms of the ligand. Generally these distances are determined by computer as discussed herein and in McRee, 1993. However distances can be determined manually once the three dimensional model is made. More commonly, the atoms of the ligand and the atoms of interacting amino acids are 3 to 4 angstroms apart. A ligand can also interact with distant amino acids, after chemical modification of the ligand to create a new ligand. Distant amino acids are generally not in contact with the ligand before chemical modification. A chemical modification can change the structure of the ligand to make a new ligand that interacts with a distant amino acid usually at least 4.5 angstroms away from the ligand. Distant amino acids rarely line the surface of the binding cavity for the ligand, as they are too far away from the ligand to be part of a pocket or surface of the binding cavity.

A compound designed or selected as binding to an NR polypeptide (in one embodiment a CAR LBD polypeptide) can be further computationally optimized so that in its bound state it would lack repulsive electrostatic interaction with the target polypeptide. Such non-complementary (e.g., electrostatic) interactions include repulsive charge-charge, dipole-dipole, and charge-dipole interactions. Specifically, the sum of all electrostatic interactions between the ligand and the polypeptide when the ligand is bound to an NR LBD make a neutral or favorable contribution to the enthalpy of binding.

One of several methods can be used to screen chemical entities or fragments for their ability to associate with a NR LBD and, more particularly, with the individual binding sites of a NR LBD, such as a ligand-binding pocket or an accessory binding site. This process can begin by visual inspection of, for example, a ligand-binding pocket on a computer screen based on the CAR LBD atomic coordinates disclosed in Tables 2-3. Selected fragments or chemical entities can then be positioned in a variety of orientations, or docked, within an individual binding site of a CAR LBD as defined herein above. Docking can be accomplished using software programs such as those available under the trade names QUANTA™ (available from Accelrys Inc, San Diego, Calif., United States of America) and SYBYL™ (available from Tripos, Inc., St. Louis, Mo., United States of America), followed by energy minimization and molecular dynamics with standard molecular mechanics force fields, such as CHARM (Brooks et al., 1993) and AMBER 5 (Case et al., 1997; Pearlman et al., 1995).

Specialized computer programs can also assist in the process of selecting fragments or chemical entities. These include:

1. GRID™ program, version 17 (Goodford, 1985), which is available from Molecular Discovery Ltd. of Oxford, United Kingdom;

2. MCSS™ program (Miranker & Karplus, 1991), which is available from Accelrys Inc, San Diego, Calif., United States of America;

3. AUTODOCK™ 3.0 program (Goodsell & Olsen, 1990), which is available from the Scripps Research Institute, La Jolla, Calif., United States of America;

4. DOCK™ 4.0 program (Kuntz et al., 1992), which is available from the University of California, San Francisco, Calif., United States of America;

5. FLEX-X™ program (See Rarey et al., 1996), which is available from Tripos, Inc., St. Louis, Mo., United States of America;

6. MVP program (Lambert, 1997); and

7. LUDI™ program (Bohm, 1992), which is available from Accelrys Inc, San Diego, Calif., United States of America.

Once suitable chemical entities or fragments have been selected, they can be assembled into a single compound or ligand. Assembly can proceed by visual inspection of the relationship of the fragments to each other on the three-dimensional image displayed on a computer screen in relation to the structure coordinates of a CAR LBD in complex with a co-regulator, optionally in further complex with a ligand. Manual model building using software such as QUANTA™ or SYBYL™ typically follows.

Useful programs to aid one of ordinary skill in the art in connecting the individual chemical entities or fragments include:

1. CAVEAT™ program (Bartlett et al., 1989), which is available from the University of California, Berkeley, Calif., United States of America;

2. 3D Database systems, such as MACCS-3D™ system program, which is available from MDL Information Systems, San Leandro, Calif., United States of America. This area is reviewed in Martin, 1992; and

3. HOOK™ program (Eisen et al., 1994), which is available from Accelrys Inc, San Diego, Calif., United States of America.

Instead of proceeding to build a NR LBD polypeptide ligand (in one embodiment a CAR LBD ligand) in a step-wise fashion one fragment or chemical entity at a time as described above, ligand compounds can be designed as a whole or de novo using the structural coordinates of a crystalline CAR LBD polypeptide of the present invention and either an empty binding site or optionally including some portion(s) of a known ligand(s). Applicable methods can employ the following software programs:

1. LUDI™ program (Bohm, 1992), which is available from Accelrys Inc, San Diego, Calif., United States of America;

2. LEGEND™ program (Nishibata & Itai, 1991); and

3. LEAPFROG™, which is available from Tripos Associates, St. Louis, Mo., United States of America.

Other molecular modeling techniques can also be employed in accordance with this invention. See e.g., Cohen et al., 1990; Navia & Murcko, 1992; and U.S. Pat. No. 6,008,033 to Abdel-Meauid et al., all of which are incorporated herein by reference.

Once a compound has been designed or selected by the above methods, the efficiency with which that compound can bind to a NR LBD can be tested and optimized by computational evaluation. By way of a particular example, a compound that has been designed or selected to function as a CAR LBD ligand can traverse a volume not overlapping that occupied by the binding site when it is bound to its native ligand. Additionally, an effective NR LBD ligand can demonstrate a relatively small difference in energy between its bound and free states (i.e., a small deformation energy of binding). Thus, the most efficient NR LBD ligands can be designed with a deformation energy of binding of in one embodiment not greater than about 10 kcal/mole, and in another embodiment not greater than 7 kcal/mole. It is possible for NR LBD ligands to interact with the polypeptide in more than one conformation that is similar in overall binding energy. In those cases, the deformation energy of binding is taken to be the difference between the energy of the free compound and the thermodynamic average energy of the conformations observed when the ligand binds to the polypeptide.

A compound designed or selected as binding to a NR LBD polypeptide (preferably a CAR polypeptide, more preferably a CAR LBD polypeptide) can be further computationally optimized so that in its bound state it would preferably lack repulsive electrostatic interaction with the target polypeptide. Such non-complementary (e.g., electrostatic) interactions include repulsive charge-charge, dipole-dipole, and charge-dipole interactions. Specifically, the sum of all electrostatic interactions between the ligand and the polypeptide when the ligand is bound to a NR LBD preferably make a neutral or favorable contribution to the enthalpy of binding.

Specific computer software is available in the art to evaluate compound deformation energy and electrostatic interaction. Examples of programs designed for such uses include:

1. GAUSSIAN 98™, which is available from Gaussian, Inc., Pittsburgh, Pa., United States of America;

2. AMBER™ program, version 6.0, which is available from the University of California, San Francisco, Calif., United States of America;

3. QUANTA™ program, which is available from Accelrys Inc, San Diego, Calif., United States of America;

4. CHARMM® program, which is available from Accelrys Inc, San Diego, Calif., United States of America; and

4. INSIGHT II® program, which is available from Accelrys Inc, San Diego, Calif. United States of America.

These programs can be implemented using a suitable computer system. Other hardware systems and software packages will be apparent to those skilled in the art after review of the disclosure of the present invention presented herein.

Once a NR LBD modulating compound has been optimally selected or designed, as described above, substitutions can then be made in some of its atoms or side groups in order to improve or modify its binding properties. In some cases, initial substitutions might be conservative, e.g., the replacement group will have approximately the same size, shape, hydrophobicity, and charge as the original group. In other cases, the replacement group will have different properties as desired to make specific interactions with the protein. Such substituted chemical compounds can then be analyzed for efficiency of fit to a NR LBD binding site using the same computer-based approaches described in detail above.

X.C. Sterically Similar Compounds A further aspect of the present invention is that sterically similar compounds can be formulated to mimic the key portions of a CAR LBD structure. Such compounds are functional equivalents. The generation of a structural functional equivalent can be achieved by the techniques of modeling and chemical design known to those of skill in the art and described herein. Modeling and chemical design of CAR and CAR LBD structural equivalents can be based on the structure coordinates of a crystalline CAR LBD polypeptide of the present invention. It will be understood that all such sterically similar constructs fall within the scope of the present invention.

XI. CAR Polypeptides

The generation of mutant and chimeric CAR polypeptides is also an aspect of the present invention. A chimeric polypeptide can comprise a CAR LBD polypeptide or a portion of a CAR LBD, (e.g. a CAR LBD) which is fused to a candidate polypeptide or a suitable region of the candidate polypeptide. Throughout the present disclosure it is intended that the term “mutant” encompass not only mutants of a CAR LBD polypeptide but chimeric proteins generated using a CAR LBD as well. It is thus intended that the following discussion of mutant CAR LBDs apply mutatis mutandis to chimeric CAR and CAR LBD polypeptides and to structural equivalents thereof.

In accordance with the present invention, a mutation can be directed to a particular site or combination of sites of a wild-type CAR LBD. For example, an accessory binding site or the binding pocket can be chosen for mutagenesis. Similarly, a residue having a location on, at or near the surface of the polypeptide can be replaced, resulting in an altered surface charge of one or more charge units, as compared to the wild-type CAR and CAR LBD. Alternatively, an amino acid residue in a CAR or a CAR LBD can be chosen for replacement based on its hydrophilic or hydrophobic characteristics.

Such mutants can be characterized by any one of several different properties as compared with the wild-type CAR LBD. For example, such mutants can have an altered surface charge of one or more charge units, or can have an increase in overall stability. Other mutants can have altered ligand specificity in comparison with, or a higher specific activity than, a wild type CAR or CAR LBD.

CAR and CAR LBD mutants of the present invention can be generated in a number of ways. For example, the wild-type sequence of a CAR or a CAR LBD can be mutated at those sites identified using this invention as desirable for mutation by employing oligonucleotide-directed mutagenesis or other conventional methods. Alternatively, mutants of a CAR or a CAR LBD can be generated by the site-specific replacement of a particular amino acid with an unnaturally occurring amino acid. In addition, CAR or CAR LBD mutants can be generated through replacement of an amino acid residue, for example, a particular cysteine or methionine residue, with selenocysteine or selenomethionine. This can be achieved by growing a host organism capable of expressing either the wild type or mutant polypeptide on a growth medium depleted of either natural cysteine or methionine (or both) but enriched in selenocysteine or selenomethionine (or both).

Mutations can be introduced into a DNA sequence coding for a CAR or a CAR LBD using synthetic oligonucleotides. These oligonucleotides contain nucleotide sequences flanking the desired mutation sites. Mutations can be generated in the full-length DNA sequence of a CAR or a CAR LBD or in any sequence coding for polypeptide fragments of a CAR or a CAR LBD.

According to the present invention, a mutated CAR or CAR LBD DNA sequence produced by the methods described above, or any alternative methods known in the art, can be expressed using an expression vector. An expression vector, as is well known to those of skill in the art, typically includes elements that permit autonomous replication in a host cell independent of the host genome, and one or more phenotypic markers for selection purposes. Either prior to or after insertion of the DNA sequences surrounding the desired CAR or CAR LBD mutant coding sequence, an expression vector includes control sequences encoding a promoter, operator, ribosome binding site, translation initiation signal, and, optionally, a repressor gene or various activator genes and a signal for termination. Where secretion of the produced mutant is desired, nucleotides encoding a “signal sequence” can be inserted prior to a CAR or a CAR LBD mutant coding sequence. For expression under the direction of the control sequences, a desired DNA sequence is operatively linked to the control sequences; that is, the sequence has an appropriate start signal in front of the DNA sequence encoding the CAR or CAR LBD mutant, and the correct reading frame to permit expression of that sequence under the control of the control sequences and production of the desired product encoded by that CAR or CAR LBD sequence.

Any of a wide variety of well-known available expression vectors can be used to express a mutated CAR or CAR LBD coding sequences of this invention. These include for example, vectors consisting of segments of chromosomal, non-chromosomal, and synthetic DNA sequences, such as known derivatives of SV40, known bacterial plasmids, e.g., plasmids from E. coli including colE1, pCR1, pBR322, pMB9 and their derivatives, wider host range plasmids, e.g., RP4, phage DNAs, e.g., derivatives of phage X, e.g., NM 989, and other DNA phages, e.g., M13 and filamentous single stranded DNA phages, yeast plasmids and vectors derived from combinations of plasmids and phage DNAs, such as plasmids which have been modified to employ phage DNA or other expression control sequences. In one embodiment of the present invention, a vector amenable to expression in a pRSETA-based expression system is employed. The pRSETA expression system is available from Invitrogen, Inc., Carlsbad, Calif., United States of America.

In addition, any of a wide variety of expression control sequences—i.e. sequences that control the expression of a DNA sequence when operatively linked to it—can be used in these vectors to express the mutated DNA sequences according to this invention. Such useful expression control sequences, include, but are not limited to the early and late promoters of SV40 for animal cells; the lac system, the trp system, the TAC or TRC system, the major operator and promoter regions of phage λ, and the control regions of fd coat protein for E. coli; the promoter for 3-phosphoglycerate kinase or other glycolytic enzymes, the promoters of acid phosphatase, (for example, Pho5), and the promoters of the yeast α-mating factors for yeast; as well as other sequences known to control the expression of genes of prokaryotic or eukaryotic cells or their viruses, and various combinations thereof.

A wide variety of hosts can be employed for producing mutated CAR and CAR LBD polypeptides according to this invention. These hosts include, for example, bacteria, such as E. coli, Bacillus, and Streptomyces; fungi, such as yeasts; animal cells, such as CHO and COS-1 cells; plant cells; insect cells, such as Sf9 cells; and transgenic host cells.

It should be understood that not all expression vectors and expression systems function in the same way to express mutated DNA sequences of this invention, and to produce modified CAR and CAR LBD polypeptides or CAR or CAR LBD mutants. Neither do all hosts function equally well with the same expression system. One of skill in the art can, however, make a selection among these vectors, expression control sequences and hosts without undue experimentation and without departing from the scope of this invention. For example, an important consideration in selecting a vector will be the ability of the vector to replicate in a given host. The copy number of the vector, the ability to control that copy number, and the expression of any other proteins encoded by the vector, such as antibiotic markers, should also be considered.

In selecting an expression control sequence, a variety of factors should also be considered. These include, for example, the relative strength of the system, its controllability and its compatibility with the DNA sequence encoding a modified CAR or CAR LBD polypeptide of this invention, with particular regard to the formation of potential secondary and tertiary structures.

Hosts should be selected by consideration of their compatibility with the chosen vector, the toxicity of a modified CAR or CAR LBD to them, their ability to express mature products, their ability to fold proteins correctly, their fermentation requirements, the ease of purification of a modified CAR or CAR LBD and safety. Within these parameters, one of skill in the art can select various vector/expression control system/host combinations that will produce useful amounts of a mutant CAR or CAR LBD. A mutant CAR or CAR LBD produced in these systems can be purified by a variety of conventional steps and strategies, including those used to purify the wild type CAR or CAR LBD.

Once a CAR LBD mutation(s) has been generated in the desired location, such as an active site or dimerization site, the mutants can be tested for any one of several properties of interest. For example, mutants can be screened for an altered charge at physiological pH. This is determined by measuring the mutant CAR or CAR LBD isoelectric point (pI) and comparing the observed value with that of the wild-type parent. Isoelectric point can be measured by gel-electrophoresis according to the method of Wellner, 1971. A mutant CAR or CAR LBD polypeptide containing a replacement amino acid located at the surface of the enzyme, as provided by the structural information of this invention, can lead to an altered surface charge and an altered pI.

XI.A. Generation of an Engineered CAR LBD or CAR LBD Mutant

In an embodiment of the present invention, a unique CAR or CAR LBD polypeptide is generated. Such a mutant can facilitate purification and the study of the ligand-binding abilities of a CAR polypeptide.

As used in the following discussion, the terms “engineered CAR”, “engineered CAR LBD”, “CAR mutant”, and “CAR LBD mutant” refers to polypeptides having amino acid sequences which contain at least one mutation in the wild-type sequence. The terms also refer to CAR and CAR LBD polypeptides which are capable of exerting a biological effect in that they comprise all or a part of the amino acid sequence of an engineered CAR or CAR LBD polypeptide of the present invention, or cross-react with antibodies raised against an engineered CAR or CAR LBD polypeptide, or retain all or some or an enhanced degree of the biological activity of the engineered CAR or CAR LBD amino acid sequence or protein. Such biological activity can include the binding of small molecules in general, and the binding of Compound 1, in particular.

The terms “engineered CAR LBD” and “CAR LBD mutant” also includes analogs of an engineered CAR LBD or CAR LBD polypeptide. By “analog” is intended that a DNA or polypeptide sequence can contain alterations relative to the sequences disclosed herein, yet retain all or some or an enhanced degree of the biological activity of those sequences. Analogs can be derived from genomic nucleotide sequences or from other organisms, or can be created synthetically. Those of skill in the art will appreciate that other analogs, as yet undisclosed or undiscovered, can be used to design and/or construct CAR LBD or CAR LBD mutant analogs. There is no need for a CAR LBD or CAR LBD mutant polypeptide to comprise all or substantially all of the amino acid sequence of SEQ ID NOs: 2 or 4. Shorter or longer sequences can be employed in the invention; shorter sequences are herein referred to as “segments”. Thus, the terms “engineered CAR LBD” and “CAR LBD mutant” also includes fusion, chimeric or recombinant CAR LBD or CAR LBD mutant polypeptides and proteins comprising sequences of the present invention. Methods of preparing such proteins are disclosed herein above and are known in the art.

XI.A.1. Sequences That Are Substantially Identical to a CAR or CAR LBD Mutant Sequence of the Present Invention

Nucleic acids that are substantially identical to a nucleic acid sequence of a CAR or CAR LBD mutant of the present invention, e.g. allelic variants, genetically altered versions of the gene, etc., bind to a CAR or CAR LBD mutant sequence under stringent hybridization conditions. By using probes, particularly labeled probes of DNA sequences, one can isolate homologous or related genes. The source of homologous genes can be any organism, including, but not limited to primates; rodents, such as rats and mice; canines; felines; bovines; equines; yeast; and nematodes.

Among mammalian species, e.g. human and mouse, homologs can have substantial sequence similarity, i.e. at least 75% sequence identity between nucleotide sequences. Sequence similarity is calculated based on a reference sequence, which can be a subset of a larger sequence, such as a conserved motif, coding region, flanking region, etc. In one embodiment, a reference sequence is at least about 18 nucleotides (nt) long, in another embodiment at least about 30 nt long, and can extend to the complete sequence that is being compared. Algorithms for sequence analysis are known in the art, such as BLAST, described in Altschul et al., 1990.

Percent identity or percent similarity of a DNA or peptide sequence can be determined, for example, by comparing sequence information using the GAP computer program, available from the University of Wisconsin Genetics Computer Group (now part of Accelrys Inc, San Diego, Calif., United States of America). The GAP program utilizes the alignment method of Needleman et al., 1970, as revised by Smith et al., 1981. Briefly, the GAP program defines similarity as the number of aligned symbols (i.e., nucleotides or amino acids) that are similar, divided by the total number of symbols in the shorter of the two sequences. The preferred parameters for the GAP program are the default parameters, which do not impose a penalty for end gaps. See e.g., Schwartz et al., 1979; Gribskov et al., 1986.

The term “similarity” is contrasted with the term “identity”. Similarity is defined as above; “identity”, however, refers to a nucleic acid or amino acid sequence having the same amino acid at the same relative position in a given family member of a gene family. Homology and similarity are generally viewed as broader terms than the term identity. Biochemically similar amino acids, for example leucine/isoleucine or glutamate/aspartate, can be present at the same position—these are not identical per se, but are biochemically “similar.” As disclosed herein, these are referred to as conservative differences or conservative substitutions. This differs from a conservative mutation at the DNA level, which changes the nucleotide sequence without making a change in the encoded amino acid, e.g. TCC to TCA, both of which encode serine.

As used herein, DNA analog sequences are “substantially identical” to specific DNA sequences disclosed herein if: (a) the DNA analog sequence is derived from coding regions of the nucleic acid sequence shown in SEQ ID NOs: 1 or 3; or (b) the DNA analog sequence is capable of hybridization with DNA sequences of (a) under stringent conditions and which encode a biologically active CAR or CAR LBD gene product; or (c) the DNA sequences are degenerate as a result of alternative genetic code to the DNA analog sequences defined in (a) and/or (b). Substantially identical analog proteins and nucleic acids will have between about 70% and 80%, preferably between about 81% to about 90% or even more preferably between about 91% and 99% sequence identity with the corresponding sequence of the native protein or nucleic acid. Sequences having lesser degrees of identity but comparable biological activity are considered to be equivalents.

As used herein, “stringent conditions” refers to conditions of high stringency, for example 6×SSC, 0.2% polyvinylpyrrolidone, 0.2% Ficoll, 0.2% bovine serum albumin, 0.1% sodium dodecyl sulfate, 100 μg/ml salmon sperm DNA and 15% formamide at 68° C. For the purposes of specifying additional conditions of high stringency, preferred conditions comprise a salt concentration of about 200 mM and temperature of about 45° C. One example of stringent conditions is hybridization in 4×SSC, at 65° C., followed by a washing in 0.1×SSC at 65° C. for one hour. Another exemplary stringent hybridization scheme uses 50% formamide, 4×SSC at 42° C.

In contrast, nucleic acids having sequence similarity are detected by hybridization under lower stringency conditions. Thus, sequence identity can be determined by hybridization under lower stringency conditions, for example, at 50° C. or higher and 0.1×SSC (9 mM NaCl/0.9 mM sodium citrate) and the sequences will remain bound when subjected to washing at 55° C. in 1×SSC.

XI.A.2. Complementarity and Hybridization to an Engineered CAR or CAR LBD Mutant Sequence

As used herein, the term “functionally equivalent codon” is used to refer to codons that encode the same amino acid, such as the ACG and AGU codons for serine. CAR or CAR LBD-encoding nucleic acid sequences comprising SEQ ID NOs: 1 and 3, which have functionally equivalent codons are covered by the present invention. Thus, when referring to the sequence examples presented in SEQ ID NOs: 1 and 3, applicants contemplate substitution of functionally equivalent codons into the sequence example of SEQ ID NOs: 1 and 3. Thus, applicants are in possession of amino acid and nucleic acids sequences which include such substitutions but which are not set forth herein in their entirety for convenience.

It will also be understood by those of skill in the art that amino acid and nucleic acid sequences can include additional residues, such as additional N— or C-terminal amino acids or 5′ or 3′ nucleic acid sequences, and yet still be essentially as set forth in one of the sequences disclosed herein, so long as the sequence retains biological protein activity where polypeptide expression is concerned. The addition of terminal sequences particularly applies to nucleic acid sequences which can, for example, include various non-coding sequences flanking either of the 5′ or 3′ portions of the coding region or can include various internal sequences, i.e., introns, which are known to occur within genes.

XI.B. Biological Equivalents

The present invention envisions and includes biological equivalents of CAR or CAR LBD mutant polypeptide of the present invention. The term “biological equivalent” refers to proteins having amino acid sequences which are substantially identical to the amino acid sequence of a CAR LBD mutant of the present invention and which are capable of exerting a biological effect in that they are capable of binding a small molecule, binding a co-regulator, homo- or heterodimerizing or cross-reacting with anti-CAR or CAR LBD mutant antibodies raised against a mutant CAR or CAR LBD polypeptide of the present invention.

For example, certain amino acids can be substituted for other amino acids in a protein structure without appreciable loss of interactive capacity with, for example, structures in the nucleus of a cell. Since it is the interactive capacity and nature of a protein that defines that protein's biological functional activity, certain amino acid sequence substitutions can be made in a protein sequence (or the nucleic acid sequence encoding it) to obtain a protein with the same, enhanced, or antagonistic properties. Such properties can be achieved by interaction with the normal targets of the protein, but this need not be the case, and the biological activity of the invention is not limited to a particular mechanism of action. It is thus in accordance with the present invention that various changes can be made in the amino acid sequence of a CAR or CAR LBD mutant polypeptide of the present invention or its underlying nucleic acid sequence without appreciable loss of biological utility or activity.

Biologically equivalent polypeptides, as used herein, are polypeptides in which certain, but not most or all, of the amino acids can be substituted. Thus, when referring to the sequence examples presented in SEQ ID NOs: 2 and 4, applicants envision substitution of codons that encode biologically equivalent amino acids, as described herein, into the sequence example of SEQ ID NOs: 2 and 4, respectively. Thus, applicants are in possession of amino acid and nucleic acids sequences which include such substitutions but which are not set forth herein in their entirety for convenience.

Alternatively, functionally equivalent proteins or peptides can be created via the application of recombinant DNA technology, in which changes in the protein structure can be engineered, based on considerations of the properties of the amino acids being exchanged, e.g. substitution of Ile for Leu. Changes designed by man can be introduced through the application of site-directed mutagenesis techniques, e.g., to introduce improvements to the antigenicity of the protein or to test a CAR or CAR LBD mutant polypeptide of the present invention in order to modulate co-regulator-binding or other activity, at the molecular level.

Amino acid substitutions, such as those which might be employed in modifying a CAR or CAR LBD mutant polypeptide of the present invention are generally, but not necessarily, based on the relative similarity of the amino acid side-chain substituents, for example, their hydrophobicity, hydrophilicity, charge, size, and the like. An analysis of the size, shape and type of the amino acid side-chain substituents reveals that arginine, lysine and histidine are all positively charged residues; that alanine, glycine and serine are all of similar size; and that phenylalanine, tryptophan and tyrosine all have a generally similar shape. Therefore, based upon these considerations, arginine, lysine and histidine; alanine, glycine and serine; and phenylalanine, tryptophan and tyrosine; are defined herein as biologically functional equivalents. Those of skill in the art will appreciate other biologically functional equivalent changes. It is implicit in the above discussion, however, that one of skill in the art can appreciate that a radical, rather than a conservative substitution is warranted in a given situation. Non-conservative substitutions in mutant CAR or CAR LBD polypeptides of the present invention are also an aspect of the present invention.

In making biologically functional equivalent amino acid substitutions, the hydropathic index of amino acids can be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art (Kyte & Doolittle, 1982, incorporated herein by reference). It is known that certain amino acids can be substituted for other amino acids having a similar hydropathic index or score and still retain a similar biological activity. In making changes based upon the hydropathic index, the substitution of amino acids whose hydropathic indices are within ±2 of the original value is preferred, those within ±1 of the original value are particularly preferred, and those within ±0.5 of the original value are even more particularly preferred.

It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with its immunogenicity and antigenicity, i.e. with a biological property of the protein. It is understood that an amino acid can be substituted for another having a similar hydrophilicity value and still obtain a biologically equivalent protein.

As detailed in U.S. Pat. No. 4,554,101 to Hopp, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, the substitution of amino acids whose hydrophilicity values are within ±2 of the original value is preferred, those that are within ±1 of the original value are particularly preferred, and those within ±0.5 of the original value are even more particularly preferred.

While discussion has focused on functionally equivalent polypeptides arising from amino acid changes, it will be appreciated that these changes can be effected by alteration of the encoding DNA, taking into consideration also that the genetic code is degenerate and that two or more codons can code for the same amino acid.

Thus, it will also be understood that this invention is not limited to the particular amino acid and nucleic acid sequences of SEQ ID NOs: 14. Recombinant vectors and isolated DNA segments can therefore variously include a CAR or CAR LBD mutant polypeptide-encoding region itself, include coding regions bearing selected alterations or modifications in the basic coding region, or include larger polypeptides which nevertheless comprise a CAR or CAR LBD mutant polypeptide-encoding regions or can encode biologically functional equivalent proteins or polypeptides which have variant amino acid sequences. Biological activity of a CAR or CAR LBD mutant polypeptide can be determined, for example, by employing binding assays known to those of skill in the art.

The nucleic acid segments of the present invention, regardless of the length of the coding sequence itself, can be combined with other DNA sequences, such as promoters, enhancers, polyadenylation signals, additional restriction enzyme sites, multiple cloning sites, other coding segments, polyhistidine encoding segments and the like, such that their overall length can vary considerably. It is therefore contemplated that a nucleic acid fragment of almost any length can be employed, with the total length preferably being limited by the ease of preparation and use in the intended recombinant DNA protocol. For example, nucleic acid fragments can be prepared which include a short stretch complementary to a nucleic acid sequence set forth in SEQ ID NOs: 1 and 3, such as about 10 nucleotides, and which are up to 10,000 or 5,000 base pairs in length. DNA segments with total lengths of about 4,000, 3,000, 2,000, 1,000, 500, 200, 100, and about 50 base pairs in length are also useful.

The DNA segments of the present invention encompass biologically functional equivalents of CAR or CAR LBD mutant polypeptides. Such sequences can arise as a consequence of codon redundancy and functional equivalency that are known to occur naturally within nucleic acid sequences and the proteins thus encoded. Alternatively, functionally equivalent proteins or polypeptides can be created via the application of recombinant DNA technology, in which changes in the protein structure can be engineered, based on considerations of the properties of the amino acids being exchanged. Changes can be introduced through the application of site-directed mutagenesis techniques, e.g., to introduce improvements to the antigenicity of the protein or to test variants of a CAR or CAR LBD mutant of the present invention in order to examine the degree of lipid-binding activity, or other activity at the molecular level. Various site-directed mutagenesis techniques are known to those of skill in the art and can be employed in the present invention.

The invention further encompasses fusion proteins and peptides wherein a CAR or CAR LBD mutant coding region of the present invention is aligned within the same expression unit with other proteins or peptides having desired functions, such as for purification or immunodetection purposes.

Recombinant vectors form important further aspects of the present invention. Particularly useful vectors are those in which the coding portion of the DNA segment is positioned under the control of a promoter. The promoter can be that naturally associated with a CAR gene, as can be obtained by isolating the 5′ non-coding sequences located upstream of the coding segment or exon, for example, using recombinant cloning and/or PCR technology and/or other methods known in the art, in conjunction with the compositions disclosed herein.

In other embodiments, certain advantages can be gained by positioning the coding DNA segment under the control of a recombinant, or heterologous, promoter. As used herein, a recombinant or heterologous promoter is a promoter that is not normally associated with a CAR gene in its natural environment. Such promoters can include promoters isolated from bacterial, viral, eukaryotic, or mammalian cells. Naturally, it will be important to employ a promoter that effectively directs the expression of the DNA segment in the cell type chosen for expression. The use of promoter and cell type combinations for protein expression is generally known to those of skill in the art of molecular biology (See e.g., Sambrook & Russell, 2001, specifically incorporated herein by reference). The promoters employed can be constitutive or inducible and can be used under the appropriate conditions to direct high level expression of the introduced DNA segment, such as is advantageous in the large-scale production of recombinant proteins or peptides. One exemplary promoter system contemplated for use in high-level expression is a T7 promoter-based system.

XII. The Role of the Three-Dimensional Structure of the CAR LDB in Solving Additional CAR Crystals

Because polypeptides can crystallize in more than one crystal form, the structural coordinates of a CAR LBD, or portions thereof, in complex with a co-regulator as provided by the present invention, are particularly useful in solving the structure of other crystal forms of CAR and the crystalline forms of other NRs and CARs. The coordinates provided in the present invention can also be used to solve the structure of CAR or CAR LBD mutants (such as those above), CAR LDB co-complexes, or the crystalline form of any other protein with significant amino acid sequence homology to any functional domain of CAR.

One method that can be employed for the purpose of solving additional CAR crystal structures is molecular replacement. See generally, Rossmann, 1972. In the molecular replacement method, an unknown crystal form, whether it is another crystal form of a CAR or a CAR LBD, (i.e. a CAR or a CAR LBD mutant), a CAR or a CAR LBD polypeptide in complex with another compound (i.e. a “co-complex”) or the crystal of some other protein with significant amino acid sequence homology to any functional region of the CAR LBD (e.g. another NR), can be determined using the CAR LBD structure coordinates provided in Tables 2-3. This method provides an accurate structural form for the unknown crystal more quickly and efficiently than attempting to determine such information ab initio.

In addition, in accordance with this invention, CAR or CAR LBD mutants can be crystallized in complex with known modulators, such as a co-regulator. The crystal structures of a series of such complexes can then be solved by molecular replacement and compared with that of wild-type CAR or the wild-type CAR. LBD. Potential sites for modification within the various binding sites of the enzyme can thus be conveniently identified. This information provides an additional tool for identifying efficient binding interactions, for example, increased hydrophobic interactions between the CAR LBD and a chemical entity or compound.

All of the complexes referred to in the present disclosure can be studied using X-ray diffraction techniques (See e.g., Blundell & Johnson, 1985) and can be refined using computer software, such as the X-PLOR™ program (Bringer, 1992; X-PLOR is available from Accelrys Inc, San Diego, Calif., United States of America). This information can thus be used to optimize known classes of CAR and CAR LBD ligands, and more importantly, to design and synthesize novel classes of CAR and CAR LBD ligands, including co-regulators.

### EXAMPLES

The following Examples have been included to illustrate exemplary modes of the invention. Certain aspects of the following Examples are described in terms of techniques and procedures found or contemplated by the present inventors to work well in the practice of the invention. These Examples are exemplified through the use of standard laboratory practices of the inventors. In light of the present disclosure and the general level of skill in the art, those of skill will appreciate that the following Examples are intended to be exemplary only and that numerous changes, modifications, and alterations can be employed without departing from the spirit and scope of the invention.

### Example 1

### Protein Expression and Purification

A DNA fragment encoding residues 103-348 of a human CAR polypeptide (GenBank Accession No. Z30425) was amplified by the polymerase chain reaction (PCR) with a commercial kit (Stratagene, La Jolla, Calif., United States of America). The 5′ PCR primer included an N-terminal poly-histidine tag sequence (MKKGHHHHHHG; SEQ ID NO: 5) along with an NdeI endonuclease restriction site (CATATG), and the 3′ PCR primer contained a BamHI restriction site (GGATCC). The PCR primers used were 5′-CGGCGGCGCCATATGAAAAAAGGTCATCATCATCATCATCATGGTCCT GTGMCTGAGTMGGAGCMG-3′ (SEQ ID NO: 6) and 5′-CGGCGGCGCGGATCCTTAGCTGCAGATCTCCTGGAGCAGCGG 3′ (SEQ ID NO: 7). The amplified DNA fragment was inserted downstream of a T7 promoter from the pRSETA vector (Invitrogen Corp., Carlsbad, Calif., United States of America) at the NdeI-BamHI enzyme restriction sites. E. coli cells BL21 (DE3) transformed with the above expression vector were grown on a carbenicillin antibiotic agar plate (50 mg/L carbenicillin). A starter culture of 80 ml LB media (10 g/L Bacto-Tryptone, 5 g/L yeast extract, 5 g/L NaCl, QC with distilled water) with carbenicillin antibiotic (50 mg/L carbenicillin) was grown from one colony at 37° C., 250 rpm for four hours. Twelve 2 L shaker flasks with 1 L LB media and carbenicillin antibiotic (50 mg/L carbenicillin) were inoculated with 5 ml of the starter culture. Cells were grown at 23° C., 250 rpm for 16 hours to an OD₆₀₀ of 2.0, and harvested by centrifugation. The pellet was completely resuspended with 20 ml extract buffer (150 mM NaCl, 50 mM imidazole pH 7.5) per liter of cells. The cells were sonicated for 5 minutes using a Sonicator Ultrasonic Processor XL-2015 (Heat Systems, Inc., Farmingdale, N.Y., United States of America) at 0° C. The lysed cells were centrifuged at 40,000 g for 40 minutes and the supernatant was loaded on a 50 ml Ni-agarose column. The column was washed with 250 ml Buffer A (50 mM imidazole pH 7.5, 150 mM NaCl), 100 ml of Buffer B (200 mM imidazole pH 7.5, 150 mM NaCl), and the protein eluted with a 300 ml gradient to Buffer B (500 mM imidazole pH 7.5, 150 mM NaCl). The peak, which eluted at 45% Buffer B, contained 60 mg of His-tagged CAR LBD protein.

This protein was diluted 5-fold in 10 mM Tris-Cl pH 8.0 to reduce the NaCl concentration before loading the entire sample on a 50 ml SP Sepharose FASTFLOW™ column (Pharmacia Biotech, now part of Amersham Biosciences Corp., Piscataway, N.J., United States of America). The column was washed with 200 ml Buffer S-A (10 mM Tris-Cl pH 8.0, 30 mM NaCl, 5 mM DTT, 1 mM EDTA pH 8.0) and the His-tagged CAR protein was eluted from the column by running a 300 ml increasing NaCl concentration gradient of Buffer S-B (10 mM Tris-Cl pH 8.0, 500 mM NaCl, 5 mM DTT, 1 mM EDTA pH 8.0). Peak fractions containing the CAR protein were pooled together, protein was concentrated to 1 mg/ml in CENTRIPREP™ 30 units (Millipore Corp., Bedford, Mass., United States of America) concentrators. The protein yield was 4 mg/L cells grown. The protein was aliquoted into 10 mg aliquots at 1.0 mg/ml and stored on ice.

The purified CAR LBD protein (10 mg) was complexed with Compound 1 (10 mM in DMSO) in a 1:5 molar ratio and incubated on ice for 1 hour. The CAR LBD/Compound 1 protein complex was concentrated to 4 mg/ml in a CENTRIPREP™ 30 units and stored on ice until needed for crystallization efforts.

### Example 2

### Crystallization and Data Collection

CAR/Compound 1 crystals were grown at 4° C. in hanging drops containing 1 μl of the protein-ligand solutions disclosed in Example 1, and 1 μl of well buffer (100-400 mM sodium potassium tartrate, pH 7.1-7.4). Crystals grew to a size of 100-200 μm within several weeks. Before data collection, crystals were transiently mixed with the well buffer that contains an additional 14% ethylene glycol, 7% glycerol, and then flash frozen in liquid nitrogen.

Orthorhombic CAR/ligand crystals formed in the P2₁2₁2₁, space group, with a=82.3 Å, b=116.8 Å, c=131.9 Å. Each asymmetric unit contained four CAR LBDs and four ligands. The crystals had a solvent content of 40%.

Crystals were screened with a Rigaku R-Axis IV detector (Rigaku International Corp., Tokyo, Japan), and data sets were collected with a MAR CCD detector at the IMCA 171D beam line at Argonne National Labs (Argonne, Ill., United States of America). The observed reflections were reduced, merged, and scaled with DENZO™ and SCALEPACK™ software in the HKL2000 package (Otwinowski, 1993).

### Example 3

### Structure Determination and Refinement

Structures were determined by molecular replacement methods with the CCP4 AMORE™ program (Collaborative Computational Project, 1994; Navaza, 1994) using the poly-alanine model of the conserved region of VDR LBD. Coordinates for this model are presented in Table 3.

The best fitting solution generated with the AMORE™ program gave a correlation coefficiency of 30% and an R-factor of 50%. The phases generated from molecular replacement were extensively refined and improved with solvent flattening, histogram matching, and NCS as implemented in CCP4DM and DMMULTI programs (Cowtan, 1994). Model building proceeded with QUANTA™ (available from Accelrys Inc, San Diego, Calif., United States of America), and refinement progressed with CNX (Brünger et al., 1998), and involved multiple cycles of manual rebuilding.

The structure of CAR in complex with the antagonist Compound 1 was determined. The statistics of the structure are summarized in Table 1.

### Example 4

### Computational Analysis

Surface area was calculated with the Connolly MS program (Connolly, 1983) and the MVP program (Lambert, 1997). The binding pocket volumes were calculated with the program GRASP (Nicholls et al., 1991), using the program MVP to close openings to solvent. The sequence alignments were generated with the MVP program.

### Example 5

### Antagonist Assays

Screening of synthetic compound libraries with the purified CAR LBD protein by a Fluorescence Resonance Energy Transfer (FRET) Ligand Sensing Assay (Parks et al., 1999) was conducted to identify molecules that alter the basal interaction between a coactivator peptide and the CAR LBD protein. Briefly, the purified human CAR LBD protein was biotinylated and labeled with streptavidin-conjugated fluorophore allophycocyanin. The labeled CAR LBD protein was incubated with a test compound and with a peptide that included the second LXXLL binding motif of the nuclear coactivator SRC-1 (GenBank Accession No. U59302; amino acids 676-700) that was labeled with europium chelate. Data were collected with a WALLAC VICTOR™ fluorescence reader (available from PerkinElmer Life Sciences Inc., Boston, Mass., United States of America) in a time resolved mode and the fluorescence ratio calculated. Compound 1 was identified from the screen to be an inverse agonist molecule that reduces the basal fluorescent signal indicating that the CAR LBD/SRC-1 interaction was reduced below background levels. Standard dose response curves were conducted with the CAR LBD protein plus Compound 1 and the EC₅₀ was determined to be 15 nM.

### Example 6

### Synthesis of Compound 1

2-(benzhydrylamino)-1-(2-phenylethyl)-1H-benzimidazole-6-carboxamide (Compound 1) was synthesized as follows. A solution of 3-fluoro-4-nitrobenzoic acid (1.28 g; 6.9 mmol) in 10 mL anhydrous N,N-dimethylformamide was treated with [O-(7-azabenzotriazol-1-yl)-1,1,3,3-tetramethyluronium hexafluoro-phosphate] (2.6 g; 6.9 mmol) followed by N,N-diisopropylethylamine (3.6 ml, 20.7 mmol). After shaking for 5 min, the mixture was added to polystyrene Rink amide AM resin (1.0 g; 0.69 mmol/g; 0.69 mmol), and the reaction was rotated at 25° C. for 18 h. The reaction solution was drained, and the resin was washed sequentially with N,N-dimethylformamide (3×), dichloromethane (3×), methanol (2×), and dichloromethane (3×). The dried resin was treated with 15.2 ml of a 0.5 M phenethylamine in N-methylpyrrolidinone solution then rotated at 70° C. for 15 hours. The cooled reaction was drained, and the resin was washed sequentially with N,N-dimethylformamide (3×), dichloromethane (3×), methanol (2×), and dichloromethane (3×). The resin was treated with 3.8 ml of 2.0 M SnCl₂.dihydrate in N-methylpyrrolidinone solution and rotated at 25° C. for 24 hours. The reaction was drained and the resin washed sequentially with 30% ethylenediamine (3×), N,N-dimethylformamide (3×), dichloromethane (3×), methanol (2×), and dichloromethane (3×). The dried diamine resin was treated with 7.6 ml of a 0.5 M benzyhydryl isothiocyanate in N-methylpyrrolidinone solution and 7.6 ml of a 1.0 M diisopropylcarbodiimide in N-methylpyrrolidinone solution. After rotating at 80° C. for 24 h the reaction was cooled to 25° C., drained, and the resin was washed sequentially with N,N-dimethylformamide (3×), dichloromethane (3×), methanol (2×), and dichloromethane (3×). The resin was treated with 30 ml 95% trifluoroacetic acid (TFA) in water and rotated at 25° C. for 3 hours. The resin was drained and washed with dichloromethane. The filtrate was concentrated in vacuo to give an oil. The oil was redissolved in dichloromethane and the solution was washed twice with saturated sodium bicarbonate (NaHCO₃). The organic layer was dried (Na₂SO₄), filtered, and concentrated in vacuo. The crude product was triturated with Et₂O/hexanes, and the solid was collected by filtration to give 333 mg (98% yield) of the title compound as an off-white solid: ¹H NMR (DMSO-d6, 400 MHz) δ 7.68 (m, 2 H), 7.63 (d, 1 H, J=8.4), 7.54 (dd, 1 H, J=8.0, 1.2), 7.40-7.00 (m, 17 H), 6.36 (d, 1 H, J=8), 4.42 (t, 2 H, J=7.4), 2.97 (t, 2 H, J=7.4); MS (ESP+) m/e 447 (MH⁺).

TABLE 2
Atomic Structure Coordinate Data Obtained From
X-ray Diffraction From the Ligand-binding Domain of CAR
In Complex With Compound 1

| | | | | | | | | | | | |
|---|---|---|---|---|---|---|---|---|---|---|---|
| ATOM | 1 | N | LEU | A | 120 | 34.417 | 18.787 | 67.312 | 1.00 | 50.31 | N |
| ATOM | 2 | CA | LEU | A | 120 | 34.298 | 17.304 | 67.212 | 1.00 | 49.96 | C |
| ATOM | 3 | C | LEU | A | 120 | 33.672 | 16.891 | 65.886 | 1.00 | 49.44 | C |
| ATOM | 4 | O | LEU | A | 120 | 32.815 | 17.592 | 65.344 | 1.00 | 49.49 | O |
| ATOM | 5 | CB | LEU | A | 120 | 33.447 | 16.756 | 68.363 | 1.00 | 50.64 | C |
| ATOM | 6 | CG | LEU | A | 120 | 34.003 | 16.880 | 69.783 | 1.00 | 51.38 | C |
| ATOM | 7 | CD1 | LEU | A | 120 | 32.969 | 16.374 | 70.777 | 1.00 | 51.56 | C |
| ATOM | 8 | CD2 | LEU | A | 120 | 35.297 | 16.085 | 69.906 | 1.00 | 51.43 | C |
| ATOM | 9 | N | ARG | A | 121 | 34.106 | 15.745 | 65.375 | 1.00 | 48.14 | N |
| ATOM | 10 | CA | ARG | A | 121 | 33.599 | 15.221 | 64.117 | 1.00 | 47.01 | C |
| ATOM | 11 | C | ARG | A | 121 | 33.113 | 13.790 | 64.314 | 1.00 | 45.50 | C |
| ATOM | 12 | O | ARG | A | 121 | 33.775 | 12.836 | 63.905 | 1.00 | 45.36 | O |
| ATOM | 13 | CB | ARG | A | 121 | 34.700 | 15.264 | 63.052 | 1.00 | 48.45 | C |
| ATOM | 14 | CG | ARG | A | 121 | 35.233 | 16.664 | 62.790 | 1.00 | 49.89 | C |
| ATOM | 15 | CD | ARG | A | 121 | 36.430 | 16.655 | 61.852 | 1.00 | 52.32 | C |
| ATOM | 16 | NE | ARG | A | 121 | 36.100 | 16.133 | 60.529 | 1.00 | 53.49 | N |
| ATOM | 17 | CZ | ARG | A | 121 | 36.947 | 16.112 | 59.504 | 1.00 | 54.08 | C |
| ATOM | 18 | NH1 | ARG | A | 121 | 38.178 | 16.586 | 59.648 | 1.00 | 54.50 | N |
| ATOM | 19 | NH2 | ARG | A | 121 | 36.563 | 15.620 | 58.334 | 1.00 | 54.12 | N |
| ATOM | 20 | N | PRO | A | 122 | 31.946 | 13.622 | 64.955 | 1.00 | 43.87 | N |
| ATOM | 21 | CA | PRO | A | 122 | 31.403 | 12.282 | 65.187 | 1.00 | 42.99 | C |
| ATOM | 22 | C | PRO | A | 122 | 31.173 | 11.529 | 63.881 | 1.00 | 42.25 | C |
| ATOM | 23 | O | PRO | A | 122 | 30.823 | 12.125 | 62.862 | 1.00 | 42.01 | O |
| ATOM | 24 | CB | PRO | A | 122 | 30.105 | 12.561 | 65.944 | 1.00 | 42.59 | C |
| ATOM | 25 | CG | PRO | A | 122 | 29.699 | 13.908 | 65.437 | 1.00 | 43.60 | C |
| ATOM | 26 | CD | PRO | A | 122 | 31.010 | 14.655 | 65.429 | 1.00 | 43.27 | C |
| ATOM | 27 | N | LYS | A | 123 | 31.379 | 10.218 | 63.920 | 1.00 | 41.53 | N |
| ATOM | 28 | CA | LYS | A | 123 | 31.205 | 9.378 | 62.744 | 1.00 | 41.30 | C |
| ATOM | 29 | C | LYS | A | 123 | 29.732 | 9.158 | 62.431 | 1.00 | 40.35 | C |
| ATOM | 30 | O | LYS | A | 123 | 28.877 | 9.250 | 63.313 | 1.00 | 39.21 | O |
| ATOM | 31 | CB | LYS | A | 123 | 31.885 | 8.024 | 62.965 | 1.00 | 42.56 | C |
| ATOM | 32 | CG | LYS | A | 123 | 33.371 | 8.127 | 63.279 | 1.00 | 45.26 | C |
| ATOM | 33 | CD | LYS | A | 123 | 33.979 | 6.761 | 63.564 | 1.00 | 46.98 | C |
| ATOM | 34 | CE | LYS | A | 123 | 35.463 | 6.876 | 63.882 | 1.00 | 47.93 | C |
| ATOM | 35 | NZ | LYS | A | 123 | 36.066 | 5.558 | 64.225 | 1.00 | 49.23 | N |
| ATOM | 36 | N | LEU | A | 124 | 29.439 | 8.879 | 61.165 | 1.00 | 39.48 | N |
| ATOM | 37 | CA | LEU | A | 124 | 28.071 | 8.622 | 60.744 | 1.00 | 38.64 | C |
| ATOM | 38 | C | LEU | A | 124 | 27.606 | 7.325 | 61.384 | 1.00 | 38.41 | C |
| ATOM | 39 | O | LEU | A | 124 | 28.293 | 6.308 | 61.304 | 1.00 | 39.12 | O |
| ATOM | 40 | CB | LEU | A | 124 | 27.996 | 8.491 | 59.220 | 1.00 | 37.76 | C |
| ATOM | 41 | CG | LEU | A | 124 | 28.162 | 9.776 | 58.406 | 1.00 | 37.83 | C |
| ATOM | 42 | CD1 | LEU | A | 124 | 28.401 | 9.438 | 56.941 | 1.00 | 37.98 | C |
| ATOM | 43 | CD2 | LEU | A | 124 | 26.922 | 10.633 | 58.564 | 1.00 | 36.97 | C |
| ATOM | 44 | N | SER | A | 125 | 26.448 | 7.362 | 62.029 | 1.00 | 38.47 | N |
| ATOM | 45 | CA | SER | A | 125 | 25.905 | 6.168 | 62.661 | 1.00 | 39.40 | C |
| ATOM | 46 | C | SER | A | 125 | 25.496 | 5.197 | 61.561 | 1.00 | 40.52 | C |
| ATOM | 47 | O | SER | A | 125 | 25.386 | 5.581 | 60.395 | 1.00 | 39.53 | O |
| ATOM | 48 | CB | SER | A | 125 | 24.679 | 6.523 | 63.495 | 1.00 | 39.88 | C |
| ATOM | 49 | OG | SER | A | 125 | 23.619 | 6.951 | 62.660 | 1.00 | 40.18 | O |
| ATOM | 50 | N | GLU | A | 126 | 25.271 | 3.940 | 61.923 | 1.00 | 41.33 | N |
| ATOM | 51 | CA | GLU | A | 126 | 24.865 | 2.956 | 60.930 | 1.00 | 42.41 | C |
| ATOM | 52 | C | GLU | A | 126 | 23.535 | 3.385 | 60.314 | 1.00 | 41.49 | C |
| ATOM | 53 | O | GLU | A | 126 | 23.313 | 3.207 | 59.115 | 1.00 | 41.40 | O |
| ATOM | 54 | CB | GLU | A | 126 | 24.727 | 1.573 | 61.573 | 1.00 | 45.02 | C |
| ATOM | 55 | CG | GLU | A | 126 | 24.325 | 0.463 | 60.605 | 1.00 | 48.95 | C |
| ATOM | 56 | CD | GLU | A | 126 | 25.202 | 0.414 | 59.361 | 1.00 | 51.93 | C |
| ATOM | 57 | OE1 | GLU | A | 126 | 24.878 | 1.105 | 58.366 | 1.00 | 53.34 | O |
| ATOM | 58 | OE2 | GLU | A | 126 | 26.222 | −0.308 | 59.379 | 1.00 | 53.64 | O |
| ATOM | 59 | N | GLU | A | 127 | 22.659 | 3.960 | 61.133 | 1.00 | 40.27 | N |
| ATOM | 60 | CA | GLU | A | 127 | 21.358 | 4.412 | 60.650 | 1.00 | 39.52 | C |
| ATOM | 61 | C | GLU | A | 127 | 21.512 | 5.550 | 59.647 | 1.00 | 37.38 | C |
| ATOM | 62 | O | GLU | A | 127 | 20.814 | 5.594 | 58.630 | 1.00 | 36.24 | O |
| ATOM | 63 | CB | GLU | A | 127 | 20.481 | 4.891 | 61.807 | 1.00 | 41.53 | C |
| ATOM | 64 | CG | GLU | A | 127 | 19.091 | 5.320 | 61.363 | 1.00 | 45.78 | C |
| ATOM | 65 | CD | GLU | A | 127 | 18.236 | 5.832 | 62.504 | 1.00 | 47.87 | C |
| ATOM | 66 | OE1 | GLU | A | 127 | 18.572 | 6.890 | 63.075 | 1.00 | 49.93 | O |
| ATOM | 67 | OE2 | GLU | A | 127 | 17.227 | 5.173 | 62.832 | 1.00 | 50.45 | O |
| ATOM | 68 | N | GLN | A | 128 | 22.420 | 6.473 | 59.939 | 1.00 | 34.92 | N |
| ATOM | 69 | CA | GLN | A | 128 | 22.654 | 7.603 | 59.052 | 1.00 | 33.94 | C |
| ATOM | 70 | C | GLN | A | 128 | 23.239 | 7.134 | 57.721 | 1.00 | 34.19 | C |
| ATOM | 71 | O | GLN | A | 128 | 22.905 | 7.671 | 56.665 | 1.00 | 32.45 | O |
| ATOM | 72 | CB | GLN | A | 128 | 23.573 | 8.622 | 59.735 | 1.00 | 33.20 | C |
| ATOM | 73 | CG | GLN | A | 128 | 22.861 | 9.410 | 60.835 | 1.00 | 32.00 | C |
| ATOM | 74 | CD | GLN | A | 128 | 23.785 | 10.317 | 61.629 | 1.00 | 32.20 | C |
| ATOM | 75 | OE1 | GLN | A | 128 | 23.346 | 11.326 | 62.192 | 1.00 | 33.66 | O |
| ATOM | 76 | NE2 | GLN | A | 128 | 25.061 | 9.960 | 61.691 | 1.00 | 30.80 | N |
| ATOM | 77 | N | GLN | A | 129 | 24.101 | 6.124 | 57.768 | 1.00 | 33.75 | N |
| ATOM | 78 | CA | GLN | A | 129 | 24.692 | 5.591 | 56.545 | 1.00 | 35.00 | C |
| ATOM | 79 | C | GLN | A | 129 | 23.588 | 4.965 | 55.702 | 1.00 | 34.31 | C |
| ATOM | 80 | O | GLN | A | 129 | 23.562 | 5.111 | 54.479 | 1.00 | 33.78 | O |
| ATOM | 81 | CB | GLN | A | 129 | 25.747 | 4.531 | 56.874 | 1.00 | 37.89 | C |
| ATOM | 82 | CG | GLN | A | 129 | 26.977 | 5.078 | 57.579 | 1.00 | 42.41 | C |
| ATOM | 83 | CD | GLN | A | 129 | 27.983 | 3.995 | 57.929 | 1.00 | 45.15 | C |
| ATOM | 84 | OE1 | GLN | A | 129 | 28.998 | 4.261 | 58.575 | 1.00 | 46.46 | O |
| ATOM | 85 | NE2 | GLN | A | 129 | 27.704 | 2.766 | 57.504 | 1.00 | 46.27 | N |
| ATOM | 86 | N | ARG | A | 130 | 22.674 | 4.270 | 56.370 | 1.00 | 33.44 | N |
| ATOM | 87 | CA | ARG | A | 130 | 21.556 | 3.614 | 55.703 | 1.00 | 34.05 | C |
| ATOM | 88 | C | ARG | A | 130 | 20.653 | 4.638 | 55.018 | 1.00 | 32.98 | C |
| ATOM | 89 | O | ARG | A | 130 | 20.226 | 4.436 | 53.881 | 1.00 | 31.44 | O |
| ATOM | 90 | CB | ARG | A | 130 | 20.759 | 2.794 | 56.723 | 1.00 | 37.04 | C |
| ATOM | 91 | CG | ARG | A | 130 | 19.497 | 2.141 | 56.184 | 1.00 | 41.36 | C |
| ATOM | 92 | CD | ARG | A | 130 | 18.961 | 1.108 | 57.171 | 1.00 | 45.69 | C |
| ATOM | 93 | NE | ARG | A | 130 | 17.642 | 0.608 | 56.790 | 1.00 | 49.25 | N |
| ATOM | 94 | CZ | ARG | A | 130 | 16.510 | 1.291 | 56.938 | 1.00 | 51.46 | C |
| ATOM | 95 | NH1 | ARG | A | 130 | 16.529 | 2.510 | 57.465 | 1.00 | 52.76 | N |
| ATOM | 96 | NH2 | ARG | A | 130 | 15.357 | 0.757 | 56.556 | 1.00 | 52.73 | N |
| ATOM | 97 | N | ILE | A | 131 | 20.367 | 5.735 | 55.712 | 1.00 | 31.16 | N |
| ATOM | 98 | CA | ILE | A | 131 | 19.519 | 6.790 | 55.158 | 1.00 | 30.41 | C |
| ATOM | 99 | C | ILE | A | 131 | 20.120 | 7.343 | 53.865 | 1.00 | 29.21 | C |
| ATOM | 100 | O | ILE | A | 131 | 19.414 | 7.528 | 52.872 | 1.00 | 27.86 | O |
| ATOM | 101 | CB | ILE | A | 131 | 19.334 | 7.945 | 56.177 | 1.00 | 31.61 | C |
| ATOM | 102 | CG1 | ILE | A | 131 | 18.513 | 7.448 | 57.372 | 1.00 | 32.47 | C |
| ATOM | 103 | CG2 | ILE | A | 131 | 18.657 | 9.138 | 55.507 | 1.00 | 31.13 | C |
| ATOM | 104 | CD1 | ILE | A | 131 | 18.287 | 8.496 | 58.457 | 1.00 | 33.63 | C |
| ATOM | 105 | N | ILE | A | 132 | 21.424 | 7.601 | 53.876 | 1.00 | 28.81 | N |
| ATOM | 106 | CA | ILE | A | 132 | 22.094 | 8.124 | 52.691 | 1.00 | 29.13 | C |
| ATOM | 107 | C | ILE | A | 132 | 22.029 | 7.115 | 51.544 | 1.00 | 29.37 | C |
| ATOM | 108 | O | ILE | A | 132 | 21.786 | 7.486 | 50.394 | 1.00 | 28.72 | O |
| ATOM | 109 | CB | ILE | A | 132 | 23.570 | 8.468 | 52.994 | 1.00 | 29.90 | C |
| ATOM | 110 | CG1 | ILE | A | 132 | 23.628 | 9.625 | 53.995 | 1.00 | 30.31 | C |
| ATOM | 111 | CG2 | ILE | A | 132 | 24.306 | 8.838 | 51.708 | 1.00 | 30.32 | C |
| ATOM | 112 | CD1 | ILE | A | 132 | 25.027 | 9.997 | 54.432 | 1.00 | 31.33 | C |
| ATOM | 113 | N | ALA | A | 133 | 22.239 | 5.841 | 51.862 | 1.00 | 28.31 | N |
| ATOM | 114 | CA | ALA | A | 133 | 22.203 | 4.785 | 50.851 | 1.00 | 27.51 | C |
| ATOM | 115 | C | ALA | A | 133 | 20.820 | 4.680 | 50.213 | 1.00 | 26.94 | C |
| ATOM | 116 | O | ALA | A | 133 | 20.694 | 4.542 | 48.993 | 1.00 | 26.91 | O |
| ATOM | 117 | CB | ALA | A | 133 | 22.587 | 3.454 | 51.479 | 1.00 | 27.94 | C |
| ATOM | 118 | N | ILE | A | 134 | 19.786 | 4.739 | 51.044 | 1.00 | 26.00 | N |
| ATOM | 119 | CA | ILE | A | 134 | 18.413 | 4.659 | 50.564 | 1.00 | 25.19 | C |
| ATOM | 120 | C | ILE | A | 134 | 18.090 | 5.832 | 49.643 | 1.00 | 24.84 | C |
| ATOM | 121 | O | ILE | A | 134 | 17.490 | 5.651 | 48.585 | 1.00 | 23.10 | O |
| ATOM | 122 | CB | ILE | A | 134 | 17.416 | 4.660 | 51.742 | 1.00 | 26.47 | C |
| ATOM | 123 | CG1 | ILE | A | 134 | 17.511 | 3.331 | 52.493 | 1.00 | 27.92 | C |
| ATOM | 124 | CG2 | ILE | A | 134 | 15.997 | 4.901 | 51.239 | 1.00 | 26.56 | C |
| ATOM | 125 | CD1 | ILE | A | 134 | 16.714 | 3.297 | 53.778 | 1.00 | 29.71 | C |
| ATOM | 126 | N | LEU | A | 135 | 18.494 | 7.030 | 50.047 | 1.00 | 23.54 | N |
| ATOM | 127 | CA | LEU | A | 135 | 18.228 | 8.220 | 49.242 | 1.00 | 23.28 | C |
| ATOM | 128 | C | LEU | A | 135 | 18.987 | 8.217 | 47.914 | 1.00 | 22.05 | C |
| ATOM | 129 | O | LEU | A | 135 | 18.454 | 8.656 | 46.894 | 1.00 | 21.44 | O |
| ATOM | 130 | CB | LEU | A | 135 | 18.559 | 9.480 | 50.045 | 1.00 | 23.21 | C |
| ATOM | 131 | CG | LEU | A | 135 | 17.644 | 9.754 | 51.246 | 1.00 | 24.57 | C |
| ATOM | 132 | CD1 | LEU | A | 135 | 18.057 | 11.076 | 51.900 | 1.00 | 26.44 | C |
| ATOM | 133 | CD2 | LEU | A | 135 | 16.185 | 9.820 | 50.789 | 1.00 | 25.56 | C |
| ATOM | 134 | N | LEU | A | 136 | 20.223 | 7.725 | 47.913 | 1.00 | 22.40 | N |
| ATOM | 135 | CA | LEU | A | 136 | 20.991 | 7.675 | 46.669 | 1.00 | 23.29 | C |
| ATOM | 136 | C | LEU | A | 136 | 20.302 | 6.721 | 45.705 | 1.00 | 23.50 | C |
| ATOM | 137 | O | LEU | A | 136 | 20.191 | 6.996 | 44.512 | 1.00 | 23.31 | O |
| ATOM | 138 | CB | LEU | A | 136 | 22.424 | 7.194 | 46.920 | 1.00 | 24.60 | C |
| ATOM | 139 | CG | LEU | A | 136 | 23.395 | 8.196 | 47.549 | 1.00 | 25.56 | C |
| ATOM | 140 | CD1 | LEU | A | 136 | 24.740 | 7.518 | 47.798 | 1.00 | 26.67 | C |
| ATOM | 141 | CD2 | LEU | A | 136 | 23.555 | 9.398 | 46.628 | 1.00 | 26.04 | C |
| ATOM | 142 | N | ASP | A | 137 | 19.845 | 5.591 | 46.232 | 1.00 | 23.87 | N |
| ATOM | 143 | CA | ASP | A | 137 | 19.156 | 4.589 | 45.427 | 1.00 | 23.95 | C |
| ATOM | 144 | C | ASP | A | 137 | 17.844 | 5.152 | 44.870 | 1.00 | 23.67 | C |
| ATOM | 145 | O | ASP | A | 137 | 17.513 | 4.943 | 43.697 | 1.00 | 22.79 | O |
| ATOM | 146 | CB | ASP | A | 137 | 18.886 | 3.348 | 46.282 | 1.00 | 26.93 | C |
| ATOM | 147 | CG | ASP | A | 137 | 18.158 | 2.266 | 45.524 | 1.00 | 31.10 | C |
| ATOM | 148 | OD1 | ASP | A | 137 | 17.010 | 1.947 | 45.900 | 1.00 | 34.78 | O |
| ATOM | 149 | OD2 | ASP | A | 137 | 18.730 | 1.734 | 44.552 | 1.00 | 34.13 | O |
| ATOM | 150 | N | ALA | A | 138 | 17.105 | 5.867 | 45.714 | 1.00 | 22.31 | N |
| ATOM | 151 | CA | ALA | A | 138 | 15.836 | 6.472 | 45.312 | 1.00 | 22.31 | C |
| ATOM | 152 | C | ALA | A | 138 | 16.063 | 7.435 | 44.157 | 1.00 | 21.39 | C |
| ATOM | 153 | O | ALA | A | 138 | 15.310 | 7.445 | 43.183 | 1.00 | 20.83 | O |
| ATOM | 154 | CB | ALA | A | 138 | 15.213 | 7.219 | 46.487 | 1.00 | 23.04 | C |
| ATOM | 155 | N | HIS | A | 139 | 17.107 | 8.249 | 44.263 | 1.00 | 21.06 | N |
| ATOM | 156 | CA | HIS | A | 139 | 17.408 | 9.202 | 43.208 | 1.00 | 21.28 | C |
| ATOM | 157 | C | HIS | A | 139 | 17.814 | 8.511 | 41.905 | 1.00 | 21.64 | C |
| ATOM | 158 | O | HIS | A | 139 | 17.385 | 8.913 | 40.824 | 1.00 | 21.17 | O |
| ATOM | 159 | CB | HIS | A | 139 | 18.528 | 10.152 | 43.631 | 1.00 | 21.21 | C |
| ATOM | 160 | CG | HIS | A | 139 | 18.730 | 11.288 | 42.680 | 1.00 | 22.53 | C |
| ATOM | 161 | ND1 | HIS | A | 139 | 19.955 | 11.593 | 42.126 | 1.00 | 25.49 | N |
| ATOM | 162 | CD2 | HIS | A | 139 | 17.850 | 12.173 | 42.157 | 1.00 | 19.49 | C |
| ATOM | 163 | CE1 | HIS | A | 139 | 19.820 | 12.615 | 41.300 | 1.00 | 20.82 | C |
| ATOM | 164 | NE2 | HIS | A | 139 | 18.552 | 12.986 | 41.301 | 1.00 | 23.99 | N |
| ATOM | 165 | N | HIS | A | 140 | 18.650 | 7.479 | 42.005 | 1.00 | 21.50 | N |
| ATOM | 166 | CA | HIS | A | 140 | 19.099 | 6.760 | 40.819 | 1.00 | 22.20 | C |
| ATOM | 167 | C | HIS | A | 140 | 17.947 | 6.088 | 40.082 | 1.00 | 21.95 | C |
| ATOM | 168 | O | HIS | A | 140 | 17.997 | 5.911 | 38.861 | 1.00 | 21.87 | O |
| ATOM | 169 | CB | HIS | A | 140 | 20.153 | 5.710 | 41.193 | 1.00 | 23.76 | C |
| ATOM | 170 | CG | HIS | A | 140 | 21.398 | 6.291 | 41.787 | 1.00 | 25.80 | C |
| ATOM | 171 | ND1 | HIS | A | 140 | 21.803 | 7.585 | 41.546 | 1.00 | 27.26 | N |
| ATOM | 172 | CD2 | HIS | A | 140 | 22.341 | 5.745 | 42.591 | 1.00 | 26.22 | C |
| ATOM | 173 | CE1 | HIS | A | 140 | 22.942 | 7.814 | 42.176 | 1.00 | 26.08 | C |
| ATOM | 174 | NE2 | HIS | A | 140 | 23.291 | 6.714 | 42.817 | 1.00 | 27.71 | N |
| ATOM | 175 | N | LYS | A | 141 | 16.908 | 5.719 | 40.821 | 1.00 | 20.41 | N |
| ATOM | 176 | CA | LYS | A | 141 | 15.745 | 5.071 | 40.225 | 1.00 | 21.89 | C |
| ATOM | 177 | C | LYS | A | 141 | 14.746 | 6.078 | 39.665 | 1.00 | 21.31 | C |
| ATOM | 178 | O | LYS | A | 141 | 13.916 | 5.730 | 38.832 | 1.00 | 22.47 | O |
| ATOM | 179 | CB | LYS | A | 141 | 15.031 | 4.203 | 41.265 | 1.00 | 23.28 | C |
| ATOM | 180 | CG | LYS | A | 141 | 15.804 | 2.960 | 41.668 | 1.00 | 26.83 | C |
| ATOM | 181 | CD | LYS | A | 141 | 15.080 | 2.209 | 42.771 | 1.00 | 30.63 | C |
| ATOM | 182 | CE | LYS | A | 141 | 15.781 | 0.902 | 43.093 | 1.00 | 33.64 | C |
| ATOM | 183 | NZ | LYS | A | 141 | 15.122 | 0.206 | 44.231 | 1.00 | 36.58 | N |
| ATOM | 184 | N | THR | A | 142 | 14.840 | 7.325 | 40.107 | 1.00 | 20.65 | N |
| ATOM | 185 | CA | THR | A | 142 | 13.893 | 8.348 | 39.664 | 1.00 | 20.68 | C |
| ATOM | 186 | C | THR | A | 142 | 14.440 | 9.502 | 38.833 | 1.00 | 20.45 | C |
| ATOM | 187 | O | THR | A | 142 | 13.682 | 10.375 | 38.420 | 1.00 | 20.32 | O |
| ATOM | 188 | CB | THR | A | 142 | 13.142 | 8.935 | 40.865 | 1.00 | 20.48 | C |
| ATOM | 189 | OG1 | THR | A | 142 | 14.081 | 9.474 | 41.805 | 1.00 | 18.91 | O |
| ATOM | 190 | CG2 | THR | A | 142 | 12.326 | 7.850 | 41.546 | 1.00 | 19.94 | C |
| ATOM | 191 | N | TYR | A | 143 | 15.747 | 9.520 | 38.595 | 1.00 | 20.03 | N |
| ATOM | 192 | CA | TYR | A | 143 | 16.342 | 10.566 | 37.768 | 1.00 | 20.44 | C |
| ATOM | 193 | C | TYR | A | 143 | 17.207 | 9.895 | 36.706 | 1.00 | 20.75 | C |
| ATOM | 194 | O | TYR | A | 143 | 18.248 | 9.323 | 37.013 | 1.00 | 21.56 | O |
| ATOM | 195 | CB | TYR | A | 143 | 17.198 | 11.529 | 38.610 | 1.00 | 20.88 | C |
| ATOM | 196 | CG | TYR | A | 143 | 17.673 | 12.742 | 37.835 | 1.00 | 20.90 | C |
| ATOM | 197 | CD1 | TYR | A | 143 | 18.721 | 12.650 | 36.915 | 1.00 | 21.44 | C |
| ATOM | 198 | CD2 | TYR | A | 143 | 17.048 | 13.980 | 37.994 | 1.00 | 21.13 | C |
| ATOM | 199 | CE1 | TYR | A | 143 | 19.132 | 13.762 | 36.170 | 1.00 | 21.80 | C |
| ATOM | 200 | CE2 | TYR | A | 143 | 17.449 | 15.090 | 37.253 | 1.00 | 20.26 | C |
| ATOM | 201 | CZ | TYR | A | 143 | 18.487 | 14.978 | 36.347 | 1.00 | 22.15 | C |
| ATOM | 202 | OH | TYR | A | 143 | 18.868 | 16.077 | 35.612 | 1.00 | 21.28 | O |
| ATOM | 203 | N | ASP | A | 144 | 16.750 | 9.959 | 35.461 | 1.00 | 20.48 | N |
| ATOM | 204 | CA | ASP | A | 144 | 17.449 | 9.365 | 34.326 | 1.00 | 21.36 | C |
| ATOM | 205 | C | ASP | A | 144 | 18.428 | 10.387 | 33.751 | 1.00 | 22.06 | C |
| ATOM | 206 | O | ASP | A | 144 | 18.016 | 11.348 | 33.102 | 1.00 | 21.75 | O |
| ATOM | 207 | CB | ASP | A | 144 | 16.412 | 8.955 | 33.274 | 1.00 | 21.65 | C |
| ATOM | 208 | CG | ASP | A | 144 | 17.032 | 8.481 | 31.976 | 1.00 | 22.22 | C |
| ATOM | 209 | OD1 | ASP | A | 144 | 18.261 | 8.286 | 31.921 | 1.00 | 22.12 | O |
| ATOM | 210 | OD2 | ASP | A | 144 | 16.266 | 8.294 | 31.007 | 1.00 | 23.20 | O |
| ATOM | 211 | N | PRO | A | 145 | 19.741 | 10.183 | 33.976 | 1.00 | 21.93 | N |
| ATOM | 212 | CA | PRO | A | 145 | 20.779 | 11.094 | 33.483 | 1.00 | 23.05 | C |
| ATOM | 213 | C | PRO | A | 145 | 20.968 | 11.106 | 31.968 | 1.00 | 22.50 | C |
| ATOM | 214 | O | PRO | A | 145 | 21.754 | 11.906 | 31.451 | 1.00 | 23.61 | O |
| ATOM | 215 | CB | PRO | A | 145 | 22.026 | 10.620 | 34.225 | 1.00 | 23.45 | C |
| ATOM | 216 | CG | PRO | A | 145 | 21.809 | 9.150 | 34.297 | 1.00 | 24.95 | C |
| ATOM | 217 | CD | PRO | A | 145 | 20.347 | 9.052 | 34.700 | 1.00 | 23.26 | C |
| ATOM | 218 | N | THR | A | 146 | 20.265 | 10.224 | 31.256 | 1.00 | 22.03 | N |
| ATOM | 219 | CA | THR | A | 146 | 20.364 | 10.192 | 29.796 | 1.00 | 21.95 | C |
| ATOM | 220 | C | THR | A | 146 | 19.174 | 10.907 | 29.155 | 1.00 | 22.52 | C |
| ATOM | 221 | O | THR | A | 146 | 19.181 | 11.177 | 27.953 | 1.00 | 22.17 | O |
| ATOM | 222 | CB | THR | A | 146 | 20.433 | 8.750 | 29.233 | 1.00 | 21.96 | C |
| ATOM | 223 | OG1 | THR | A | 146 | 19.167 | 8.099 | 29.395 | 1.00 | 21.08 | O |
| ATOM | 224 | CG2 | THR | A | 146 | 21.509 | 7.949 | 29.956 | 1.00 | 23.14 | C |
| ATOM | 225 | N | TYR | A | 147 | 18.158 | 11.210 | 29.963 | 1.00 | 22.04 | N |
| ATOM | 226 | CA | TYR | A | 147 | 16.963 | 11.912 | 29.489 | 1.00 | 22.53 | C |
| ATOM | 227 | C | TYR | A | 147 | 16.313 | 11.191 | 28.309 | 1.00 | 23.10 | C |
| ATOM | 228 | O | TYR | A | 147 | 15.789 | 11.821 | 27.393 | 1.00 | 23.05 | O |
| ATOM | 229 | CB | TYR | A | 147 | 17.335 | 13.350 | 29.093 | 1.00 | 23.34 | C |
| ATOM | 230 | CG | TYR | A | 147 | 18.159 | 14.049 | 30.150 | 1.00 | 23.73 | C |
| ATOM | 231 | CD1 | TYR | A | 147 | 19.525 | 14.274 | 29.968 | 1.00 | 25.15 | C |
| ATOM | 232 | CD2 | TYR | A | 147 | 17.593 | 14.398 | 31.372 | 1.00 | 23.61 | C |
| ATOM | 233 | CE1 | TYR | A | 147 | 20.304 | 14.818 | 30.989 | 1.00 | 25.82 | C |
| ATOM | 234 | CE2 | TYR | A | 147 | 18.363 | 14.941 | 32.396 | 1.00 | 26.56 | C |
| ATOM | 235 | CZ | TYR | A | 147 | 19.716 | 15.142 | 32.199 | 1.00 | 26.11 | C |
| ATOM | 236 | OH | TYR | A | 147 | 20.484 | 15.619 | 33.237 | 1.00 | 29.64 | O |
| ATOM | 237 | N | SER | A | 148 | 16.326 | 9.862 | 28.355 | 1.00 | 23.29 | N |
| ATOM | 238 | CA | SER | A | 148 | 15.781 | 9.046 | 27.278 | 1.00 | 23.65 | C |
| ATOM | 239 | C | SER | A | 148 | 14.263 | 9.078 | 27.073 | 1.00 | 24.65 | C |
| ATOM | 240 | O | SER | A | 148 | 13.783 | 8.650 | 26.024 | 1.00 | 24.62 | O |
| ATOM | 241 | CB | SER | A | 148 | 16.243 | 7.593 | 27.450 | 1.00 | 26.66 | C |
| ATOM | 242 | OG | SER | A | 148 | 15.684 | 7.006 | 28.614 | 1.00 | 29.82 | O |
| ATOM | 243 | N | ASP | A | 149 | 13.505 | 9.576 | 28.048 | 1.00 | 22.99 | N |
| ATOM | 244 | CA | ASP | A | 149 | 12.045 | 9.632 | 27.905 | 1.00 | 23.85 | C |
| ATOM | 245 | C | ASP | A | 149 | 11.534 | 10.925 | 27.272 | 1.00 | 24.00 | C |
| ATOM | 246 | O | ASP | A | 149 | 10.371 | 11.008 | 26.879 | 1.00 | 24.41 | O |
| ATOM | 247 | CB | ASP | A | 149 | 11.349 | 9.488 | 29.263 | 1.00 | 24.47 | C |
| ATOM | 248 | CG | ASP | A | 149 | 11.517 | 8.114 | 29.872 | 1.00 | 27.05 | C |
| ATOM | 249 | OD1 | ASP | A | 149 | 11.441 | 7.116 | 29.124 | 1.00 | 26.86 | O |
| ATOM | 250 | OD2 | ASP | A | 149 | 11.707 | 8.037 | 31.105 | 1.00 | 26.29 | O |
| ATOM | 251 | N | PHE | A | 150 | 12.396 | 11.927 | 27.171 | 1.00 | 24.31 | N |
| ATOM | 252 | CA | PHE | A | 150 | 11.995 | 13.231 | 26.646 | 1.00 | 25.09 | C |
| ATOM | 253 | C | PHE | A | 150 | 11.363 | 13.263 | 25.252 | 1.00 | 25.91 | C |
| ATOM | 254 | O | PHE | A | 150 | 10.565 | 14.155 | 24.949 | 1.00 | 25.61 | O |
| ATOM | 255 | CB | PHE | A | 150 | 13.188 | 14.187 | 26.715 | 1.00 | 24.68 | C |
| ATOM | 256 | CG | PHE | A | 150 | 13.546 | 14.611 | 28.121 | 1.00 | 25.17 | C |
| ATOM | 257 | CD1 | PHE | A | 150 | 13.422 | 13.726 | 29.187 | 1.00 | 25.54 | C |
| ATOM | 258 | CD2 | PHE | A | 150 | 14.028 | 15.891 | 28.374 | 1.00 | 26.43 | C |
| ATOM | 259 | CE1 | PHE | A | 150 | 13.773 | 14.104 | 30.484 | 1.00 | 25.74 | C |
| ATOM | 260 | CE2 | PHE | A | 150 | 14.384 | 16.278 | 29.667 | 1.00 | 25.55 | C |
| ATOM | 261 | CZ | PHE | A | 150 | 14.256 | 15.386 | 30.721 | 1.00 | 24.63 | C |
| ATOM | 262 | N | CYS | A | 151 | 11.694 | 12.298 | 24.404 | 1.00 | 27.60 | N |
| ATOM | 263 | CA | CYS | A | 151 | 11.116 | 12.286 | 23.063 | 1.00 | 28.74 | C |
| ATOM | 264 | C | CYS | A | 151 | 9.640 | 11.891 | 23.094 | 1.00 | 28.90 | C |
| ATOM | 265 | O | CYS | A | 151 | 8.951 | 11.958 | 22.075 | 1.00 | 28.40 | O |
| ATOM | 266 | CB | CYS | A | 151 | 11.894 | 11.332 | 22.154 | 1.00 | 31.34 | C |
| ATOM | 267 | SG | CYS | A | 151 | 11.886 | 9.633 | 22.716 | 1.00 | 37.88 | S |
| ATOM | 268 | N | GLN | A | 152 | 9.152 | 11.482 | 24.262 | 1.00 | 27.55 | N |
| ATOM | 269 | CA | GLN | A | 152 | 7.753 | 11.093 | 24.393 | 1.00 | 27.93 | C |
| ATOM | 270 | C | GLN | A | 152 | 6.858 | 12.285 | 24.711 | 1.00 | 27.73 | C |
| ATOM | 271 | O | GLN | A | 152 | 5.633 | 12.202 | 24.590 | 1.00 | 28.51 | O |
| ATOM | 272 | CB | GLN | A | 152 | 7.602 | 10.021 | 25.473 | 1.00 | 29.61 | C |
| ATOM | 273 | CG | GLN | A | 152 | 8.312 | 8.724 | 25.123 | 1.00 | 33.35 | C |
| ATOM | 274 | CD | GLN | A | 152 | 8.121 | 7.650 | 26.173 | 1.00 | 36.62 | C |
| ATOM | 275 | OE1 | GLN | A | 152 | 6.995 | 7.260 | 26.478 | 1.00 | 39.37 | O |
| ATOM | 276 | NE2 | GLN | A | 152 | 9.225 | 7.162 | 26.732 | 1.00 | 38.35 | N |
| ATOM | 277 | N | PHE | A | 153 | 7.469 | 13.395 | 25.115 | 1.00 | 25.45 | N |
| ATOM | 278 | CA | PHE | A | 153 | 6.705 | 14.597 | 25.439 | 1.00 | 25.30 | C |
| ATOM | 279 | C | PHE | A | 153 | 6.261 | 15.273 | 24.151 | 1.00 | 25.61 | C |
| ATOM | 280 | O | PHE | A | 153 | 6.799 | 14.998 | 23.071 | 1.00 | 24.69 | O |
| ATOM | 281 | CB | PHE | A | 153 | 7.564 | 15.608 | 26.215 | 1.00 | 23.94 | C |
| ATOM | 282 | CG | PHE | A | 153 | 8.187 | 15.060 | 27.469 | 1.00 | 23.45 | C |
| ATOM | 283 | CD1 | PHE | A | 153 | 9.332 | 15.654 | 27.990 | 1.00 | 22.75 | C |
| ATOM | 284 | CD2 | PHE | A | 153 | 7.654 | 13.949 | 28.116 | 1.00 | 23.40 | C |
| ATOM | 285 | CE1 | PHE | A | 153 | 9.948 | 15.146 | 29.133 | 1.00 | 23.18 | C |
| ATOM | 286 | CE2 | PHE | A | 153 | 8.261 | 13.434 | 29.263 | 1.00 | 22.50 | C |
| ATOM | 287 | CZ | PHE | A | 153 | 9.414 | 14.037 | 29.769 | 1.00 | 22.91 | C |
| ATOM | 288 | N | ARG | A | 154 | 5.276 | 16.158 | 24.260 | 1.00 | 25.51 | N |
| ATOM | 289 | CA | ARG | A | 154 | 4.842 | 16.902 | 23.092 | 1.00 | 26.08 | C |
| ATOM | 290 | C | ARG | A | 154 | 6.094 | 17.673 | 22.689 | 1.00 | 27.20 | C |
| ATOM | 291 | O | ARG | A | 154 | 6.824 | 18.184 | 23.542 | 1.00 | 26.99 | O |
| ATOM | 292 | CB | ARG | A | 154 | 3.681 | 17.830 | 23.449 | 1.00 | 26.73 | C |
| ATOM | 293 | CG | ARG | A | 154 | 2.351 | 17.087 | 23.522 | 1.00 | 27.85 | C |
| ATOM | 294 | CD | ARG | A | 154 | 1.232 | 17.964 | 24.066 | 1.00 | 27.71 | C |
| ATOM | 295 | NE | ARG | A | 154 | 1.347 | 18.138 | 25.509 | 1.00 | 27.14 | N |
| ATOM | 296 | CZ | ARG | A | 154 | 0.497 | 18.839 | 26.248 | 1.00 | 28.47 | C |
| ATOM | 297 | NH1 | ARG | A | 154 | −0.538 | 19.444 | 25.677 | 1.00 | 29.16 | N |
| ATOM | 298 | NH2 | ARG | A | 154 | 0.673 | 18.919 | 27.560 | 1.00 | 27.66 | N |
| ATOM | 299 | N | PRO | A | 155 | 6.368 | 17.757 | 21.384 | 1.00 | 27.28 | N |
| ATOM | 300 | CA | PRO | A | 155 | 7.554 | 18.454 | 20.892 | 1.00 | 28.12 | C |
| ATOM | 301 | C | PRO | A | 155 | 7.709 | 19.929 | 21.217 | 1.00 | 28.41 | C |
| ATOM | 302 | O | PRO | A | 155 | 6.733 | 20.676 | 21.291 | 1.00 | 27.77 | O |
| ATOM | 303 | CB | PRO | A | 155 | 7.491 | 18.206 | 19.388 | 1.00 | 28.83 | C |
| ATOM | 304 | CG | PRO | A | 155 | 6.020 | 18.191 | 19.130 | 1.00 | 29.19 | C |
| ATOM | 305 | CD | PRO | A | 155 | 5.508 | 17.335 | 20.262 | 1.00 | 28.61 | C |
| ATOM | 306 | N | PRO | A | 156 | 8.956 | 20.361 | 21.437 | 1.00 | 28.25 | N |
| ATOM | 307 | CA | PRO | A | 156 | 9.202 | 21.768 | 21.739 | 1.00 | 29.56 | C |
| ATOM | 308 | C | PRO | A | 156 | 9.054 | 22.532 | 20.425 | 1.00 | 30.08 | C |
| ATOM | 309 | O | PRO | A | 156 | 9.483 | 22.054 | 19.371 | 1.00 | 30.96 | O |
| ATOM | 310 | CB | PRO | A | 156 | 10.640 | 21.763 | 22.250 | 1.00 | 29.92 | C |
| ATOM | 311 | CG | PRO | A | 156 | 11.262 | 20.646 | 21.476 | 1.00 | 30.45 | C |
| ATOM | 312 | CD | PRO | A | 156 | 10.198 | 19.573 | 21.538 | 1.00 | 29.15 | C |
| ATOM | 313 | N | VAL | A | 157 | 8.417 | 23.693 | 20.489 | 1.00 | 30.75 | N |
| ATOM | 314 | CA | VAL | A | 157 | 8.220 | 24.538 | 19.319 | 1.00 | 31.52 | C |
| ATOM | 315 | C | VAL | A | 157 | 8.764 | 25.907 | 19.692 | 1.00 | 32.33 | C |
| ATOM | 316 | O | VAL | A | 157 | 8.361 | 26.482 | 20.698 | 1.00 | 33.09 | O |
| ATOM | 317 | CB | VAL | A | 157 | 6.727 | 24.663 | 18.962 | 1.00 | 31.97 | C |
| ATOM | 318 | CG1 | VAL | A | 157 | 6.544 | 25.654 | 17.825 | 1.00 | 32.48 | C |
| ATOM | 319 | CG2 | VAL | A | 157 | 6.177 | 23.302 | 18.573 | 1.00 | 32.24 | C |
| ATOM | 320 | N | ARG | A | 158 | 9.681 | 26.425 | 18.885 | 1.00 | 33.83 | N |
| ATOM | 321 | CA | ARG | A | 158 | 10.289 | 27.716 | 19.173 | 1.00 | 36.19 | C |
| ATOM | 322 | C | ARG | A | 158 | 10.020 | 28.766 | 18.096 | 1.00 | 38.44 | C |
| ATOM | 323 | O | ARG | A | 158 | 10.763 | 28.881 | 17.123 | 1.00 | 39.20 | O |
| ATOM | 324 | CB | ARG | A | 158 | 11.794 | 27.523 | 19.367 | 1.00 | 35.86 | C |
| ATOM | 325 | CG | ARG | A | 158 | 12.131 | 26.585 | 20.524 | 1.00 | 34.74 | C |
| ATOM | 326 | CD | ARG | A | 158 | 13.606 | 26.231 | 20.561 | 1.00 | 35.06 | C |
| ATOM | 327 | NE | ARG | A | 158 | 13.991 | 25.641 | 21.841 | 1.00 | 32.63 | N |
| ATOM | 328 | CZ | ARG | A | 158 | 14.006 | 24.339 | 22.113 | 1.00 | 31.82 | C |
| ATOM | 329 | NH1 | ARG | A | 158 | 13.658 | 23.450 | 21.192 | 1.00 | 32.10 | N |
| ATOM | 330 | NH2 | ARG | A | 158 | 14.370 | 23.926 | 23.319 | 1.00 | 29.69 | N |
| ATOM | 331 | N | VAL | A | 159 | 8.949 | 29.531 | 18.284 | 1.00 | 40.67 | N |
| ATOM | 332 | CA | VAL | A | 159 | 8.568 | 30.574 | 17.338 | 1.00 | 42.44 | C |
| ATOM | 333 | C | VAL | A | 159 | 9.511 | 31.767 | 17.432 | 1.00 | 43.24 | C |
| ATOM | 334 | O | VAL | A | 159 | 10.170 | 31.968 | 18.451 | 1.00 | 42.85 | O |
| ATOM | 335 | CB | VAL | A | 159 | 7.135 | 31.066 | 17.607 | 1.00 | 42.85 | C |
| ATOM | 336 | CG1 | VAL | A | 159 | 6.147 | 29.937 | 17.367 | 1.00 | 43.48 | C |
| ATOM | 337 | CG2 | VAL | A | 159 | 7.027 | 31.577 | 19.040 | 1.00 | 43.60 | C |
| ATOM | 338 | N | ASN | A | 160 | 9.576 | 32.557 | 16.365 | 1.00 | 44.06 | N |
| ATOM | 339 | CA | ASN | A | 160 | 10.440 | 33.730 | 16.357 | 1.00 | 44.92 | C |
| ATOM | 340 | C | ASN | A | 160 | 9.876 | 34.768 | 17.320 | 1.00 | 45.24 | C |
| ATOM | 341 | O | ASN | A | 160 | 8.728 | 35.198 | 17.185 | 1.00 | 45.27 | O |
| ATOM | 342 | CB | ASN | A | 160 | 10.530 | 34.326 | 14.949 | 1.00 | 46.00 | C |
| ATOM | 343 | CG | ASN | A | 160 | 11.017 | 33.322 | 13.921 | 1.00 | 47.25 | C |
| ATOM | 344 | OD1 | ASN | A | 160 | 12.030 | 32.649 | 14.124 | 1.00 | 47.25 | O |
| ATOM | 345 | ND2 | ASN | A | 160 | 10.298 | 33.218 | 12.808 | 1.00 | 48.36 | N |
| ATOM | 346 | N | ASP | A | 161 | 10.688 | 35.156 | 18.298 | 1.00 | 45.02 | N |
| ATOM | 347 | CA | ASP | A | 161 | 10.282 | 36.142 | 19.289 | 1.00 | 44.79 | C |
| ATOM | 348 | C | ASP | A | 161 | 11.515 | 36.834 | 19.862 | 1.00 | 44.74 | C |
| ATOM | 349 | O | ASP | A | 161 | 11.679 | 36.939 | 21.077 | 1.00 | 44.64 | O |
| ATOM | 350 | CB | ASP | A | 161 | 9.483 | 35.463 | 20.406 | 1.00 | 44.26 | C |
| ATOM | 351 | CG | ASP | A | 161 | 9.101 | 36.421 | 21.515 | 1.00 | 44.34 | C |
| ATOM | 352 | OD1 | ASP | A | 161 | 8.640 | 37.540 | 21.201 | 1.00 | 43.26 | O |
| ATOM | 353 | OD2 | ASP | A | 161 | 9.258 | 36.054 | 22.700 | 1.00 | 43.90 | O |
| ATOM | 354 | N | GLY | A | 162 | 12.383 | 37.304 | 18.972 | 1.00 | 44.73 | N |
| ATOM | 355 | CA | GLY | A | 162 | 13.592 | 37.977 | 19.409 | 1.00 | 44.74 | C |
| ATOM | 356 | C | GLY | A | 162 | 13.292 | 39.196 | 20.261 | 1.00 | 44.56 | C |
| ATOM | 357 | O | GLY | A | 162 | 14.135 | 39.638 | 21.042 | 1.00 | 45.10 | O |
| ATOM | 358 | N | GLY | A | 163 | 12.086 | 39.736 | 20.116 | 1.00 | 44.30 | N |
| ATOM | 359 | CA | GLY | A | 163 | 11.706 | 40.911 | 20.879 | 1.00 | 43.74 | C |
| ATOM | 360 | C | GLY | A | 163 | 11.206 | 40.618 | 22.282 | 1.00 | 43.23 | C |
| ATOM | 361 | O | GLY | A | 163 | 11.066 | 41.533 | 23.096 | 1.00 | 43.53 | O |
| ATOM | 362 | N | GLY | A | 164 | 10.946 | 39.346 | 22.572 | 1.00 | 42.43 | N |
| ATOM | 363 | CA | GLY | A | 164 | 10.450 | 38.980 | 23.889 | 1.00 | 40.70 | C |
| ATOM | 364 | C | GLY | A | 164 | 9.094 | 39.616 | 24.130 | 1.00 | 39.47 | C |
| ATOM | 365 | O | GLY | A | 164 | 8.812 | 40.125 | 25.222 | 1.00 | 40.10 | O |
| ATOM | 366 | N | SER | A | 216 | 8.256 | 39.587 | 23.099 | 1.00 | 36.82 | N |
| ATOM | 367 | CA | SER | A | 216 | 6.918 | 40.165 | 23.162 | 1.00 | 35.37 | C |
| ATOM | 368 | C | SER | A | 216 | 5.965 | 39.359 | 24.032 | 1.00 | 34.15 | C |
| ATOM | 369 | O | SER | A | 216 | 5.653 | 38.213 | 23.721 | 1.00 | 32.50 | O |
| ATOM | 370 | CB | SER | A | 216 | 6.329 | 40.277 | 21.755 | 1.00 | 35.39 | C |
| ATOM | 371 | OG | SER | A | 216 | 4.958 | 40.634 | 21.812 | 1.00 | 35.41 | O |
| ATOM | 372 | N | VAL | A | 217 | 5.495 | 39.969 | 25.116 | 1.00 | 33.39 | N |
| ATOM | 373 | CA | VAL | A | 217 | 4.563 | 39.301 | 26.013 | 1.00 | 33.22 | C |
| ATOM | 374 | C | VAL | A | 217 | 3.299 | 38.922 | 25.251 | 1.00 | 32.19 | C |
| ATOM | 375 | O | VAL | A | 217 | 2.783 | 37.816 | 25.399 | 1.00 | 31.92 | O |
| ATOM | 376 | CB | VAL | A | 217 | 4.161 | 40.208 | 27.195 | 1.00 | 33.21 | C |
| ATOM | 377 | CG1 | VAL | A | 217 | 3.203 | 39.462 | 28.119 | 1.00 | 35.52 | C |
| ATOM | 378 | CG2 | VAL | A | 217 | 5.396 | 40.644 | 27.960 | 1.00 | 35.70 | C |
| ATOM | 379 | N | THR | A | 218 | 2.809 | 39.846 | 24.428 | 1.00 | 31.30 | N |
| ATOM | 380 | CA | THR | A | 218 | 1.597 | 39.609 | 23.653 | 1.00 | 30.58 | C |
| ATOM | 381 | C | THR | A | 218 | 1.736 | 38.398 | 22.741 | 1.00 | 30.30 | C |
| ATOM | 382 | O | THR | A | 218 | 0.852 | 37.544 | 22.695 | 1.00 | 30.29 | O |
| ATOM | 383 | CB | THR | A | 218 | 1.235 | 40.843 | 22.802 | 1.00 | 30.65 | C |
| ATOM | 384 | OG1 | THR | A | 218 | 1.025 | 41.966 | 23.667 | 1.00 | 30.30 | O |
| ATOM | 385 | CG2 | THR | A | 218 | −0.035 | 40.587 | 22.000 | 1.00 | 31.23 | C |
| ATOM | 386 | N | LEU | A | 219 | 2.849 | 38.325 | 22.018 | 1.00 | 29.44 | N |
| ATOM | 387 | CA | LEU | A | 219 | 3.095 | 37.206 | 21.117 | 1.00 | 29.87 | C |
| ATOM | 388 | C | LEU | A | 219 | 3.260 | 35.905 | 21.894 | 1.00 | 29.21 | C |
| ATOM | 389 | O | LEU | A | 219 | 2.710 | 34.869 | 21.516 | 1.00 | 29.73 | O |
| ATOM | 390 | CB | LEU | A | 219 | 4.355 | 37.462 | 20.286 | 1.00 | 31.48 | C |
| ATOM | 391 | CG | LEU | A | 219 | 4.778 | 36.321 | 19.352 | 1.00 | 33.59 | C |
| ATOM | 392 | CD1 | LEU | A | 219 | 3.700 | 36.083 | 18.301 | 1.00 | 34.93 | C |
| ATOM | 393 | CD2 | LEU | A | 219 | 6.100 | 36.676 | 18.690 | 1.00 | 35.57 | C |
| ATOM | 394 | N | GLU | A | 220 | 4.018 | 35.963 | 22.982 | 1.00 | 28.82 | N |
| ATOM | 395 | CA | GLU | A | 220 | 4.258 | 34.781 | 23.801 | 1.00 | 29.09 | C |
| ATOM | 396 | C | GLU | A | 220 | 2.958 | 34.194 | 24.342 | 1.00 | 29.07 | C |
| ATOM | 397 | O | GLU | A | 220 | 2.757 | 32.983 | 24.297 | 1.00 | 27.80 | O |
| ATOM | 398 | CB | GLU | A | 220 | 5.213 | 35.131 | 24.946 | 1.00 | 31.33 | C |
| ATOM | 399 | CG | GLU | A | 220 | 6.620 | 35.466 | 24.456 | 1.00 | 32.76 | C |
| ATOM | 400 | CD | GLU | A | 220 | 7.434 | 36.277 | 25.450 | 1.00 | 35.67 | C |
| ATOM | 401 | OE1 | GLU | A | 220 | 8.574 | 36.657 | 25.104 | 1.00 | 37.03 | O |
| ATOM | 402 | OE2 | GLU | A | 220 | 6.944 | 36.541 | 26.569 | 1.00 | 36.27 | O |
| ATOM | 403 | N | LEU | A | 221 | 2.073 | 35.052 | 24.841 | 1.00 | 28.79 | N |
| ATOM | 404 | CA | LEU | A | 221 | 0.799 | 34.592 | 25.383 | 1.00 | 29.82 | C |
| ATOM | 405 | C | LEU | A | 221 | −0.143 | 34.089 | 24.293 | 1.00 | 29.77 | C |
| ATOM | 406 | O | LEU | A | 221 | −0.923 | 33.165 | 24.516 | 1.00 | 30.04 | O |
| ATOM | 407 | CB | LEU | A | 221 | 0.125 | 35.714 | 26.181 | 1.00 | 30.05 | C |
| ATOM | 408 | CG | LEU | A | 221 | 0.743 | 36.046 | 27.544 | 1.00 | 31.65 | C |
| ATOM | 409 | CD1 | LEU | A | 221 | 0.065 | 37.278 | 28.138 | 1.00 | 32.22 | C |
| ATOM | 410 | CD2 | LEU | A | 221 | 0.588 | 34.850 | 28.482 | 1.00 | 31.89 | C |
| ATOM | 411 | N | SER | A | 222 | −0.066 | 34.687 | 23.108 | 1.00 | 31.28 | N |
| ATOM | 412 | CA | SER | A | 222 | −0.931 | 34.272 | 22.011 | 1.00 | 32.25 | C |
| ATOM | 413 | C | SER | A | 222 | −0.536 | 32.905 | 21.460 | 1.00 | 32.84 | C |
| ATOM | 414 | O | SER | A | 222 | −1.380 | 32.170 | 20.947 | 1.00 | 33.76 | O |
| ATOM | 415 | CB | SER | A | 222 | −0.895 | 35.304 | 20.877 | 1.00 | 34.81 | C |
| ATOM | 416 | OG | SER | A | 222 | 0.367 | 35.315 | 20.230 | 1.00 | 39.03 | O |
| ATOM | 417 | N | GLN | A | 223 | 0.742 | 32.558 | 21.584 | 1.00 | 31.84 | N |
| ATOM | 418 | CA | GLN | A | 223 | 1.234 | 31.288 | 21.063 | 1.00 | 31.75 | C |
| ATOM | 419 | C | GLN | A | 223 | 1.596 | 30.215 | 22.089 | 1.00 | 30.53 | C |
| ATOM | 420 | O | GLN | A | 223 | 1.306 | 29.039 | 21.869 | 1.00 | 30.69 | O |
| ATOM | 421 | CB | GLN | A | 223 | 2.434 | 31.550 | 20.151 | 1.00 | 34.71 | C |
| ATOM | 422 | CG | GLN | A | 223 | 2.066 | 32.296 | 18.873 | 1.00 | 38.65 | C |
| ATOM | 423 | CD | GLN | A | 223 | 3.275 | 32.719 | 18.065 | 1.00 | 42.46 | C |
| ATOM | 424 | OE1 | GLN | A | 223 | 3.154 | 33.114 | 16.903 | 1.00 | 45.44 | O |
| ATOM | 425 | NE2 | GLN | A | 223 | 4.450 | 32.652 | 18.679 | 1.00 | 44.57 | N |
| ATOM | 426 | N | LEU | A | 224 | 2.226 | 30.610 | 23.195 | 1.00 | 28.64 | N |
| ATOM | 427 | CA | LEU | A | 224 | 2.632 | 29.654 | 24.232 | 1.00 | 27.07 | C |
| ATOM | 428 | C | LEU | A | 224 | 3.209 | 28.401 | 23.569 | 1.00 | 26.40 | C |
| ATOM | 429 | O | LEU | A | 224 | 2.898 | 27.274 | 23.962 | 1.00 | 25.81 | O |
| ATOM | 430 | CB | LEU | A | 224 | 1.424 | 29.276 | 25.102 | 1.00 | 27.70 | C |
| ATOM | 431 | CG | LEU | A | 224 | 0.785 | 30.424 | 25.893 | 1.00 | 27.88 | C |
| ATOM | 432 | CD1 | LEU | A | 224 | −0.463 | 29.931 | 26.615 | 1.00 | 29.53 | C |
| ATOM | 433 | CD2 | LEU | A | 224 | 1.789 | 30.981 | 26.884 | 1.00 | 27.54 | C |
| ATOM | 434 | N | SER | A | 225 | 4.071 | 28.614 | 22.577 | 1.00 | 25.74 | N |
| ATOM | 435 | CA | SER | A | 225 | 4.667 | 27.531 | 21.798 | 1.00 | 25.83 | C |
| ATOM | 436 | C | SER | A | 225 | 5.454 | 26.473 | 22.563 | 1.00 | 25.18 | C |
| ATOM | 437 | O | SER | A | 225 | 5.446 | 25.302 | 22.182 | 1.00 | 25.89 | O |
| ATOM | 438 | CB | SER | A | 225 | 5.557 | 28.110 | 20.696 | 1.00 | 26.31 | C |
| ATOM | 439 | OG | SER | A | 225 | 6.710 | 28.731 | 21.233 | 1.00 | 29.36 | O |
| ATOM | 440 | N | MET | A | 226 | 6.132 | 26.880 | 23.630 | 1.00 | 24.58 | N |
| ATOM | 441 | CA | MET | A | 226 | 6.931 | 25.948 | 24.424 | 1.00 | 24.51 | C |
| ATOM | 442 | C | MET | A | 226 | 6.193 | 25.387 | 25.631 | 1.00 | 24.00 | C |
| ATOM | 443 | O | MET | A | 226 | 6.725 | 24.529 | 26.336 | 1.00 | 24.19 | O |
| ATOM | 444 | CB | MET | A | 226 | 8.219 | 26.629 | 24.905 | 1.00 | 24.70 | C |
| ATOM | 445 | CG | MET | A | 226 | 9.329 | 26.715 | 23.870 | 1.00 | 25.85 | C |
| ATOM | 446 | SD | MET | A | 226 | 9.960 | 25.094 | 23.351 | 1.00 | 27.27 | S |
| ATOM | 447 | CE | MET | A | 226 | 10.773 | 24.531 | 24.858 | 1.00 | 28.07 | C |
| ATOM | 448 | N | LEU | A | 227 | 4.969 | 25.850 | 25.872 | 1.00 | 23.05 | N |
| ATOM | 449 | CA | LEU | A | 227 | 4.225 | 25.377 | 27.030 | 1.00 | 23.85 | C |
| ATOM | 450 | C | LEU | A | 227 | 3.882 | 23.887 | 27.032 | 1.00 | 23.65 | C |
| ATOM | 451 | O | LEU | A | 227 | 4.062 | 23.218 | 28.052 | 1.00 | 24.44 | O |
| ATOM | 452 | CB | LEU | A | 227 | 2.949 | 26.212 | 27.237 | 1.00 | 24.02 | C |
| ATOM | 453 | CG | LEU | A | 227 | 2.139 | 25.868 | 28.494 | 1.00 | 24.67 | C |
| ATOM | 454 | CD1 | LEU | A | 227 | 3.019 | 25.994 | 29.730 | 1.00 | 25.75 | C |
| ATOM | 455 | CD2 | LEU | A | 227 | 0.936 | 26.798 | 28.612 | 1.00 | 25.81 | C |
| ATOM | 456 | N | PRO | A | 228 | 3.395 | 23.336 | 25.901 | 1.00 | 24.00 | N |
| ATOM | 457 | CA | PRO | A | 228 | 3.073 | 21.904 | 25.931 | 1.00 | 23.78 | C |
| ATOM | 458 | C | PRO | A | 228 | 4.261 | 21.024 | 26.330 | 1.00 | 23.69 | C |
| ATOM | 459 | O | PRO | A | 228 | 4.123 | 20.109 | 27.155 | 1.00 | 23.20 | O |
| ATOM | 460 | CB | PRO | A | 228 | 2.602 | 21.626 | 24.504 | 1.00 | 24.23 | C |
| ATOM | 461 | CG | PRO | A | 228 | 1.957 | 22.939 | 24.110 | 1.00 | 24.58 | C |
| ATOM | 462 | CD | PRO | A | 228 | 2.962 | 23.948 | 24.629 | 1.00 | 23.63 | C |
| ATOM | 463 | N | HIS | A | 229 | 5.421 | 21.305 | 25.747 | 1.00 | 22.38 | N |
| ATOM | 464 | CA | HIS | A | 229 | 6.626 | 20.532 | 26.037 | 1.00 | 22.16 | C |
| ATOM | 465 | C | HIS | A | 229 | 7.089 | 20.679 | 27.490 | 1.00 | 21.32 | C |
| ATOM | 466 | O | HIS | A | 229 | 7.409 | 19.687 | 28.151 | 1.00 | 20.38 | O |
| ATOM | 467 | CB | HIS | A | 229 | 7.765 | 20.951 | 25.103 | 1.00 | 22.65 | C |
| ATOM | 468 | CG | HIS | A | 229 | 9.037 | 20.196 | 25.337 | 1.00 | 23.54 | C |
| ATOM | 469 | ND1 | HIS | A | 229 | 9.235 | 18.910 | 24.883 | 1.00 | 24.88 | N |
| ATOM | 470 | CD2 | HIS | A | 229 | 10.160 | 20.535 | 26.012 | 1.00 | 23.99 | C |
| ATOM | 471 | CE1 | HIS | A | 229 | 10.427 | 18.488 | 25.270 | 1.00 | 25.42 | C |
| ATOM | 472 | NE2 | HIS | A | 229 | 11.009 | 19.455 | 25.957 | 1.00 | 23.32 | N |
| ATOM | 473 | N | LEU | A | 230 | 7.139 | 21.913 | 27.985 | 1.00 | 20.90 | N |
| ATOM | 474 | CA | LEU | A | 230 | 7.578 | 22.139 | 29.355 | 1.00 | 21.22 | C |
| ATOM | 475 | C | LEU | A | 230 | 6.563 | 21.623 | 30.361 | 1.00 | 21.08 | C |
| ATOM | 476 | O | LEU | A | 230 | 6.938 | 21.164 | 31.435 | 1.00 | 19.50 | O |
| ATOM | 477 | CB | LEU | A | 230 | 7.858 | 23.625 | 29.602 | 1.00 | 21.98 | C |
| ATOM | 478 | CG | LEU | A | 230 | 9.051 | 24.211 | 28.839 | 1.00 | 23.32 | C |
| ATOM | 479 | CD1 | LEU | A | 230 | 9.285 | 25.637 | 29.322 | 1.00 | 25.98 | C |
| ATOM | 480 | CD2 | LEU | A | 230 | 10.311 | 23.371 | 29.073 | 1.00 | 24.89 | C |
| ATOM | 481 | N | ALA | A | 231 | 5.279 | 21.703 | 30.022 | 1.00 | 20.40 | N |
| ATOM | 482 | CA | ALA | A | 231 | 4.243 | 21.197 | 30.917 | 1.00 | 21.50 | C |
| ATOM | 483 | C | ALA | A | 231 | 4.421 | 19.685 | 31.040 | 1.00 | 21.12 | C |
| ATOM | 484 | O | ALA | A | 231 | 4.303 | 19.124 | 32.129 | 1.00 | 21.78 | O |
| ATOM | 485 | CB | ALA | A | 231 | 2.859 | 21.522 | 30.361 | 1.00 | 22.95 | C |
| ATOM | 486 | N | ASP | A | 232 | 4.707 | 19.028 | 29.919 | 1.00 | 20.87 | N |
| ATOM | 487 | CA | ASP | A | 232 | 4.910 | 17.582 | 29.916 | 1.00 | 21.48 | C |
| ATOM | 488 | C | ASP | A | 232 | 6.168 | 17.228 | 30.711 | 1.00 | 20.43 | C |
| ATOM | 489 | O | ASP | A | 232 | 6.167 | 16.259 | 31.463 | 1.00 | 21.59 | O |
| ATOM | 490 | CB | ASP | A | 232 | 5.022 | 17.056 | 28.482 | 1.00 | 21.87 | C |
| ATOM | 491 | CG | ASP | A | 232 | 3.664 | 16.893 | 27.807 | 1.00 | 25.14 | C |
| ATOM | 492 | OD1 | ASP | A | 232 | 3.639 | 16.665 | 26.582 | 1.00 | 26.65 | O |
| ATOM | 493 | OD2 | ASP | A | 232 | 2.623 | 16.982 | 28.497 | 1.00 | 25.49 | O |
| ATOM | 494 | N | LEU | A | 233 | 7.228 | 18.018 | 30.549 | 1.00 | 21.20 | N |
| ATOM | 495 | CA | LEU | A | 233 | 8.483 | 17.785 | 31.278 | 1.00 | 20.50 | C |
| ATOM | 496 | C | LEU | A | 233 | 8.267 | 17.940 | 32.785 | 1.00 | 20.58 | C |
| ATOM | 497 | O | LEU | A | 233 | 8.755 | 17.139 | 33.587 | 1.00 | 18.39 | O |
| ATOM | 498 | CB | LEU | A | 233 | 9.565 | 18.770 | 30.811 | 1.00 | 20.92 | C |
| ATOM | 499 | CG | LEU | A | 233 | 10.826 | 18.839 | 31.684 | 1.00 | 20.96 | C |
| ATOM | 500 | CD1 | LEU | A | 233 | 11.554 | 17.502 | 31.652 | 1.00 | 22.15 | C |
| ATOM | 501 | CD2 | LEU | A | 233 | 11.737 | 19.969 | 31.190 | 1.00 | 22.47 | C |
| ATOM | 502 | N | VAL | A | 234 | 7.539 | 18.981 | 33.172 | 1.00 | 20.09 | N |
| ATOM | 503 | CA | VAL | A | 234 | 7.263 | 19.217 | 34.583 | 1.00 | 20.15 | C |
| ATOM | 504 | C | VAL | A | 234 | 6.320 | 18.152 | 35.146 | 1.00 | 19.97 | C |
| ATOM | 505 | O | VAL | A | 234 | 6.500 | 17.691 | 36.268 | 1.00 | 19.99 | O |
| ATOM | 506 | CB | VAL | A | 234 | 6.665 | 20.630 | 34.796 | 1.00 | 21.02 | C |
| ATOM | 507 | CG1 | VAL | A | 234 | 6.104 | 20.778 | 36.209 | 1.00 | 23.20 | C |
| ATOM | 508 | CG2 | VAL | A | 234 | 7.754 | 21.679 | 34.566 | 1.00 | 21.83 | C |
| ATOM | 509 | N | SER | A | 235 | 5.324 | 17.749 | 34.362 | 1.00 | 18.23 | N |
| ATOM | 510 | CA | SER | A | 235 | 4.378 | 16.732 | 34.821 | 1.00 | 19.68 | C |
| ATOM | 511 | C | SER | A | 235 | 5.117 | 15.413 | 35.079 | 1.00 | 19.46 | C |
| ATOM | 512 | O | SER | A | 235 | 4.906 | 14.743 | 36.095 | 1.00 | 19.95 | O |
| ATOM | 513 | CB | SER | A | 235 | 3.284 | 16.537 | 33.767 | 1.00 | 21.15 | C |
| ATOM | 514 | OG | SER | A | 235 | 2.229 | 15.734 | 34.274 | 1.00 | 26.38 | O |
| ATOM | 515 | N | TYR | A | 236 | 5.983 | 15.057 | 34.140 | 1.00 | 19.05 | N |
| ATOM | 516 | CA | TYR | A | 236 | 6.796 | 13.849 | 34.222 | 1.00 | 19.13 | C |
| ATOM | 517 | C | TYR | A | 236 | 7.660 | 13.930 | 35.479 | 1.00 | 18.86 | C |
| ATOM | 518 | O | TYR | A | 236 | 7.792 | 12.958 | 36.223 | 1.00 | 18.39 | O |
| ATOM | 519 | CB | TYR | A | 236 | 7.675 | 13.781 | 32.976 | 1.00 | 19.07 | C |
| ATOM | 520 | CG | TYR | A | 236 | 8.800 | 12.764 | 32.990 | 1.00 | 19.18 | C |
| ATOM | 521 | CD1 | TYR | A | 236 | 8.601 | 11.466 | 32.527 | 1.00 | 20.38 | C |
| ATOM | 522 | CD2 | TYR | A | 236 | 10.084 | 13.131 | 33.391 | 1.00 | 20.55 | C |
| ATOM | 523 | CE1 | TYR | A | 236 | 9.665 | 10.557 | 32.448 | 1.00 | 21.48 | C |
| ATOM | 524 | CE2 | TYR | A | 236 | 11.149 | 12.233 | 33.321 | 1.00 | 20.66 | C |
| ATOM | 525 | CZ | TYR | A | 236 | 10.934 | 10.954 | 32.846 | 1.00 | 21.96 | C |
| ATOM | 526 | OH | TYR | A | 236 | 11.996 | 10.079 | 32.749 | 1.00 | 21.78 | O |
| ATOM | 527 | N | SER | A | 237 | 8.241 | 15.105 | 35.711 | 1.00 | 17.93 | N |
| ATOM | 528 | CA | SER | A | 237 | 9.106 | 15.312 | 36.868 | 1.00 | 18.19 | C |
| ATOM | 529 | C | SER | A | 237 | 8.373 | 15.218 | 38.199 | 1.00 | 18.73 | C |
| ATOM | 530 | O | SER | A | 237 | 8.929 | 14.737 | 39.184 | 1.00 | 19.34 | O |
| ATOM | 531 | CB | SER | A | 237 | 9.830 | 16.654 | 36.730 | 1.00 | 18.72 | C |
| ATOM | 532 | OG | SER | A | 237 | 10.648 | 16.628 | 35.573 | 1.00 | 19.76 | O |
| ATOM | 533 | N | ILE | A | 238 | 7.128 | 15.680 | 38.237 | 1.00 | 18.89 | N |
| ATOM | 534 | CA | ILE | A | 238 | 6.343 | 15.597 | 39.460 | 1.00 | 20.25 | C |
| ATOM | 535 | C | ILE | A | 238 | 6.101 | 14.119 | 39.759 | 1.00 | 20.17 | C |
| ATOM | 536 | O | ILE | A | 238 | 6.129 | 13.705 | 40.914 | 1.00 | 20.62 | O |
| ATOM | 537 | CB | ILE | A | 238 | 4.984 | 16.337 | 39.317 | 1.00 | 21.21 | C |
| ATOM | 538 | CG1 | ILE | A | 238 | 5.226 | 17.847 | 39.236 | 1.00 | 23.61 | C |
| ATOM | 539 | CG2 | ILE | A | 238 | 4.068 | 16.001 | 40.502 | 1.00 | 23.76 | C |
| ATOM | 540 | CD1 | ILE | A | 238 | 3.972 | 18.668 | 38.937 | 1.00 | 24.70 | C |
| ATOM | 541 | N | GLN | A | 239 | 5.868 | 13.315 | 38.719 | 1.00 | 20.04 | N |
| ATOM | 542 | CA | GLN | A | 239 | 5.657 | 11.890 | 38.936 | 1.00 | 19.72 | C |
| ATOM | 543 | C | GLN | A | 239 | 6.911 | 11.261 | 39.531 | 1.00 | 20.24 | C |
| ATOM | 544 | O | GLN | A | 239 | 6.823 | 10.433 | 40.437 | 1.00 | 19.92 | O |
| ATOM | 545 | CB | GLN | A | 239 | 5.288 | 11.178 | 37.628 | 1.00 | 21.35 | C |
| ATOM | 546 | CG | GLN | A | 239 | 3.920 | 11.576 | 37.086 | 1.00 | 21.87 | C |
| ATOM | 547 | CD | GLN | A | 239 | 3.487 | 10.707 | 35.922 | 1.00 | 23.58 | C |
| ATOM | 548 | OE1 | GLN | A | 239 | 3.092 | 9.556 | 36.105 | 1.00 | 26.39 | O |
| ATOM | 549 | NE2 | GLN | A | 239 | 3.568 | 11.249 | 34.720 | 1.00 | 22.31 | N |
| ATOM | 550 | N | LYS | A | 240 | 8.080 | 11.661 | 39.037 | 1.00 | 19.37 | N |
| ATOM | 551 | CA | LYS | A | 240 | 9.336 | 11.116 | 39.557 | 1.00 | 19.49 | C |
| ATOM | 552 | C | LYS | A | 240 | 9.575 | 11.583 | 40.994 | 1.00 | 20.03 | C |
| ATOM | 553 | O | LYS | A | 240 | 10.086 | 10.826 | 41.826 | 1.00 | 20.81 | O |
| ATOM | 554 | CB | LYS | A | 240 | 10.509 | 11.525 | 38.658 | 1.00 | 19.27 | C |
| ATOM | 555 | CG | LYS | A | 240 | 10.385 | 11.015 | 37.216 | 1.00 | 19.70 | C |
| ATOM | 556 | CD | LYS | A | 240 | 10.174 | 9.491 | 37.165 | 1.00 | 20.85 | C |
| ATOM | 557 | CE | LYS | A | 240 | 10.201 | 8.986 | 35.734 | 1.00 | 20.78 | C |
| ATOM | 558 | NZ | LYS | A | 240 | 9.919 | 7.527 | 35.631 | 1.00 | 21.79 | N |
| ATOM | 559 | N | VAL | A | 241 | 9.203 | 12.827 | 41.284 | 1.00 | 19.95 | N |
| ATOM | 560 | CA | VAL | A | 241 | 9.355 | 13.380 | 42.630 | 1.00 | 21.18 | C |
| ATOM | 561 | C | VAL | A | 241 | 8.466 | 12.633 | 43.621 | 1.00 | 22.58 | C |
| ATOM | 562 | O | VAL | A | 241 | 8.845 | 12.418 | 44.769 | 1.00 | 22.01 | O |
| ATOM | 563 | CB | VAL | A | 241 | 9.006 | 14.890 | 42.658 | 1.00 | 22.53 | C |
| ATOM | 564 | CG1 | VAL | A | 241 | 8.893 | 15.392 | 44.104 | 1.00 | 23.49 | C |
| ATOM | 565 | CG2 | VAL | A | 241 | 10.092 | 15.671 | 41.929 | 1.00 | 22.43 | C |
| ATOM | 566 | N | ILE | A | 242 | 7.277 | 12.237 | 43.178 | 1.00 | 22.44 | N |
| ATOM | 567 | CA | ILE | A | 242 | 6.375 | 11.492 | 44.052 | 1.00 | 23.64 | C |
| ATOM | 568 | C | ILE | A | 242 | 7.027 | 10.157 | 44.416 | 1.00 | 23.45 | C |
| ATOM | 569 | O | ILE | A | 242 | 6.987 | 9.726 | 45.573 | 1.00 | 25.50 | O |
| ATOM | 570 | CB | ILE | A | 242 | 5.012 | 11.255 | 43.360 | 1.00 | 24.32 | C |
| ATOM | 571 | CG1 | ILE | A | 242 | 4.235 | 12.575 | 43.303 | 1.00 | 25.64 | C |
| ATOM | 572 | CG2 | ILE | A | 242 | 4.214 | 10.186 | 44.104 | 1.00 | 24.95 | C |
| ATOM | 573 | CD1 | ILE | A | 242 | 3.012 | 12.540 | 42.401 | 1.00 | 25.41 | C |
| ATOM | 574 | N | GLY | A | 243 | 7.652 | 9.521 | 43.431 | 1.00 | 22.76 | N |
| ATOM | 575 | CA | GLY | A | 243 | 8.310 | 8.246 | 43.665 | 1.00 | 23.14 | C |
| ATOM | 576 | C | GLY | A | 243 | 9.491 | 8.385 | 44.604 | 1.00 | 23.29 | C |
| ATOM | 577 | O | GLY | A | 243 | 9.719 | 7.525 | 45.454 | 1.00 | 24.26 | O |
| ATOM | 578 | N | PHE | A | 244 | 10.244 | 9.471 | 44.443 | 1.00 | 22.21 | N |
| ATOM | 579 | CA | PHE | A | 244 | 11.406 | 9.754 | 45.287 | 1.00 | 23.08 | C |
| ATOM | 580 | C | PHE | A | 244 | 10.962 | 9.960 | 46.734 | 1.00 | 23.33 | C |
| ATOM | 581 | O | PHE | A | 244 | 11.509 | 9.359 | 47.665 | 1.00 | 22.96 | O |
| ATOM | 582 | CB | PHE | A | 244 | 12.110 | 11.023 | 44.799 | 1.00 | 21.55 | C |
| ATOM | 583 | CG | PHE | A | 244 | 13.264 | 11.454 | 45.663 | 1.00 | 23.20 | C |
| ATOM | 584 | CD1 | PHE | A | 244 | 14.474 | 10.764 | 45.632 | 1.00 | 25.04 | C |
| ATOM | 585 | CD2 | PHE | A | 244 | 13.140 | 12.548 | 46.516 | 1.00 | 24.78 | C |
| ATOM | 586 | CE1 | PHE | A | 244 | 15.542 | 11.157 | 46.437 | 1.00 | 25.46 | C |
| ATOM | 587 | CE2 | PHE | A | 244 | 14.205 | 12.950 | 47.327 | 1.00 | 24.71 | C |
| ATOM | 588 | CZ | PHE | A | 244 | 15.407 | 12.254 | 47.286 | 1.00 | 24.22 | C |
| ATOM | 589 | N | ALA | A | 245 | 9.963 | 10.819 | 46.912 | 1.00 | 23.25 | N |
| ATOM | 590 | CA | ALA | A | 245 | 9.441 | 11.134 | 48.233 | 1.00 | 23.37 | C |
| ATOM | 591 | C | ALA | A | 245 | 8.960 | 9.906 | 49.006 | 1.00 | 25.09 | C |
| ATOM | 592 | O | ALA | A | 245 | 9.182 | 9.805 | 50.212 | 1.00 | 24.87 | O |
| ATOM | 593 | CB | ALA | A | 245 | 8.310 | 12.156 | 48.113 | 1.00 | 22.36 | C |
| ATOM | 594 | N | LYS | A | 246 | 8.309 | 8.975 | 48.314 | 1.00 | 26.15 | N |
| ATOM | 595 | CA | LYS | A | 246 | 7.800 | 7.768 | 48.959 | 1.00 | 28.66 | C |
| ATOM | 596 | C | LYS | A | 246 | 8.914 | 6.918 | 49.562 | 1.00 | 29.21 | C |
| ATOM | 597 | O | LYS | A | 246 | 8.668 | 6.117 | 50.466 | 1.00 | 29.75 | O |
| ATOM | 598 | CB | LYS | A | 246 | 6.997 | 6.931 | 47.957 | 1.00 | 30.93 | C |
| ATOM | 599 | CG | LYS | A | 246 | 5.702 | 7.593 | 47.501 | 1.00 | 34.75 | C |
| ATOM | 600 | CD | LYS | A | 246 | 5.017 | 6.811 | 46.383 | 1.00 | 37.28 | C |
| ATOM | 601 | CE | LYS | A | 246 | 4.410 | 5.501 | 46.873 | 1.00 | 40.02 | C |
| ATOM | 602 | NZ | LYS | A | 246 | 3.230 | 5.724 | 47.756 | 1.00 | 42.15 | N |
| ATOM | 603 | N | MET | A | 247 | 10.138 | 7.104 | 49.074 | 1.00 | 28.68 | N |
| ATOM | 604 | CA | MET | A | 247 | 11.282 | 6.339 | 49.562 | 1.00 | 29.45 | C |
| ATOM | 605 | C | MET | A | 247 | 12.076 | 7.021 | 50.681 | 1.00 | 28.75 | C |
| ATOM | 606 | O | MET | A | 247 | 13.012 | 6.431 | 51.230 | 1.00 | 28.61 | O |
| ATOM | 607 | CB | MET | A | 247 | 12.219 | 5.990 | 48.396 | 1.00 | 30.97 | C |
| ATOM | 608 | CG | MET | A | 247 | 11.614 | 5.007 | 47.393 | 1.00 | 34.76 | C |
| ATOM | 609 | SD | MET | A | 247 | 12.766 | 4.475 | 46.096 | 1.00 | 39.72 | S |
| ATOM | 610 | CE | MET | A | 247 | 12.303 | 5.554 | 44.763 | 1.00 | 39.07 | C |
| ATOM | 611 | N | ILE | A | 248 | 11.709 | 8.253 | 51.023 | 1.00 | 27.31 | N |
| ATOM | 612 | CA | ILE | A | 248 | 12.391 | 8.973 | 52.100 | 1.00 | 28.07 | C |
| ATOM | 613 | C | ILE | A | 248 | 12.033 | 8.295 | 53.420 | 1.00 | 28.99 | C |
| ATOM | 614 | O | ILE | A | 248 | 10.859 | 8.179 | 53.763 | 1.00 | 28.97 | O |
| ATOM | 615 | CB | ILE | A | 248 | 11.934 | 10.446 | 52.195 | 1.00 | 27.49 | C |
| ATOM | 616 | CG1 | ILE | A | 248 | 12.299 | 11.198 | 50.916 | 1.00 | 26.51 | C |
| ATOM | 617 | CG2 | ILE | A | 248 | 12.582 | 11.114 | 53.411 | 1.00 | 28.63 | C |
| ATOM | 618 | CD1 | ILE | A | 248 | 11.730 | 12.605 | 50.865 | 1.00 | 25.47 | C |
| ATOM | 619 | N | PRO | A | 249 | 13.041 | 7.844 | 54.181 | 1.00 | 30.64 | N |
| ATOM | 620 | CA | PRO | A | 249 | 12.764 | 7.182 | 55.460 | 1.00 | 31.91 | C |
| ATOM | 621 | C | PRO | A | 249 | 11.818 | 7.992 | 56.348 | 1.00 | 32.16 | C |
| ATOM | 622 | O | PRO | A | 249 | 12.107 | 9.138 | 56.688 | 1.00 | 34.02 | O |
| ATOM | 623 | CB | PRO | A | 249 | 14.153 | 7.035 | 56.075 | 1.00 | 32.36 | C |
| ATOM | 624 | CG | PRO | A | 249 | 15.021 | 6.835 | 54.871 | 1.00 | 32.23 | C |
| ATOM | 625 | CD | PRO | A | 249 | 14.490 | 7.890 | 53.917 | 1.00 | 30.61 | C |
| ATOM | 626 | N | GLY | A | 250 | 10.686 | 7.392 | 56.706 | 1.00 | 32.30 | N |
| ATOM | 627 | CA | GLY | A | 250 | 9.725 | 8.064 | 57.565 | 1.00 | 33.31 | C |
| ATOM | 628 | C | GLY | A | 250 | 8.542 | 8.700 | 56.858 | 1.00 | 33.48 | C |
| ATOM | 629 | O | GLY | A | 250 | 7.484 | 8.888 | 57.459 | 1.00 | 33.45 | O |
| ATOM | 630 | N | PHE | A | 251 | 8.709 | 9.023 | 55.579 | 1.00 | 33.51 | N |
| ATOM | 631 | CA | PHE | A | 251 | 7.643 | 9.658 | 54.809 | 1.00 | 33.69 | C |
| ATOM | 632 | C | PHE | A | 251 | 6.335 | 8.871 | 54.833 | 1.00 | 34.57 | C |
| ATOM | 633 | O | PHE | A | 251 | 5.259 | 9.455 | 54.964 | 1.00 | 35.10 | O |
| ATOM | 634 | CB | PHE | A | 251 | 8.082 | 9.850 | 53.356 | 1.00 | 31.35 | C |
| ATOM | 635 | CG | PHE | A | 251 | 7.180 | 10.754 | 52.564 | 1.00 | 29.89 | C |
| ATOM | 636 | CD1 | PHE | A | 251 | 7.234 | 12.134 | 52.735 | 1.00 | 30.12 | C |
| ATOM | 637 | CD2 | PHE | A | 251 | 6.276 | 10.227 | 51.643 | 1.00 | 30.05 | C |
| ATOM | 638 | CE1 | PHE | A | 251 | 6.400 | 12.979 | 51.999 | 1.00 | 29.28 | C |
| ATOM | 639 | CE2 | PHE | A | 251 | 5.441 | 11.063 | 50.906 | 1.00 | 28.78 | C |
| ATOM | 640 | CZ | PHE | A | 251 | 5.505 | 12.440 | 51.085 | 1.00 | 28.48 | C |
| ATOM | 641 | N | ARG | A | 252 | 6.431 | 7.551 | 54.703 | 1.00 | 36.83 | N |
| ATOM | 642 | CA | ARG | A | 252 | 5.250 | 6.691 | 54.698 | 1.00 | 39.19 | C |
| ATOM | 643 | C | ARG | A | 252 | 4.535 | 6.647 | 56.045 | 1.00 | 39.61 | C |
| ATOM | 644 | O | ARG | A | 252 | 3.391 | 6.200 | 56.127 | 1.00 | 40.31 | O |
| ATOM | 645 | CB | ARG | A | 252 | 5.625 | 5.262 | 54.292 | 1.00 | 41.06 | C |
| ATOM | 646 | CG | ARG | A | 252 | 6.138 | 5.101 | 52.867 | 1.00 | 44.96 | C |
| ATOM | 647 | CD | ARG | A | 252 | 6.260 | 3.620 | 52.516 | 1.00 | 47.63 | C |
| ATOM | 648 | NE | ARG | A | 252 | 6.777 | 3.393 | 51.169 | 1.00 | 50.79 | N |
| ATOM | 649 | CZ | ARG | A | 252 | 8.062 | 3.459 | 50.831 | 1.00 | 51.79 | C |
| ATOM | 650 | NH1 | ARG | A | 252 | 8.982 | 3.745 | 51.745 | 1.00 | 52.82 | N |
| ATOM | 651 | NH2 | ARG | A | 252 | 8.427 | 3.235 | 49.576 | 1.00 | 52.64 | N |
| ATOM | 652 | N | ASP | A | 253 | 5.205 | 7.102 | 57.098 | 1.00 | 39.96 | N |
| ATOM | 653 | CA | ASP | A | 253 | 4.610 | 7.097 | 58.430 | 1.00 | 40.45 | C |
| ATOM | 654 | C | ASP | A | 253 | 3.648 | 8.255 | 58.635 | 1.00 | 39.90 | C |
| ATOM | 655 | O | ASP | A | 253 | 2.902 | 8.284 | 59.612 | 1.00 | 39.68 | O |
| ATOM | 656 | CB | ASP | A | 253 | 5.698 | 7.127 | 59.506 | 1.00 | 42.53 | C |
| ATOM | 657 | CG | ASP | A | 253 | 6.524 | 5.856 | 59.531 | 1.00 | 44.84 | C |
| ATOM | 658 | OD1 | ASP | A | 253 | 5.938 | 4.767 | 59.345 | 1.00 | 47.60 | O |
| ATOM | 659 | OD2 | ASP | A | 253 | 7.752 | 5.942 | 59.743 | 1.00 | 45.66 | O |
| ATOM | 660 | N | LEU | A | 254 | 3.669 | 9.208 | 57.710 | 1.00 | 38.00 | N |
| ATOM | 661 | CA | LEU | A | 254 | 2.782 | 10.361 | 57.780 | 1.00 | 37.81 | C |
| ATOM | 662 | C | LEU | A | 254 | 1.417 | 9.978 | 57.218 | 1.00 | 37.76 | C |
| ATOM | 663 | O | LEU | A | 254 | 1.293 | 9.000 | 56.476 | 1.00 | 37.49 | O |
| ATOM | 664 | CB | LEU | A | 254 | 3.348 | 11.521 | 56.955 | 1.00 | 36.51 | C |
| ATOM | 665 | CG | LEU | A | 254 | 4.707 | 12.101 | 57.346 | 1.00 | 36.86 | C |
| ATOM | 666 | CD1 | LEU | A | 254 | 5.142 | 13.113 | 56.297 | 1.00 | 35.66 | C |
| ATOM | 667 | CD2 | LEU | A | 254 | 4.620 | 12.751 | 58.719 | 1.00 | 36.85 | C |
| ATOM | 668 | N | THR | A | 255 | 0.395 | 10.745 | 57.579 | 1.00 | 38.14 | N |
| ATOM | 669 | CA | THR | A | 255 | −0.950 | 10.496 | 57.077 | 1.00 | 39.08 | C |
| ATOM | 670 | C | THR | A | 255 | −0.928 | 10.828 | 55.588 | 1.00 | 39.76 | C |
| ATOM | 671 | O | THR | A | 255 | −0.075 | 11.593 | 55.139 | 1.00 | 39.15 | O |
| ATOM | 672 | CB | THR | A | 255 | −1.982 | 11.397 | 57.781 | 1.00 | 39.68 | C |
| ATOM | 673 | OG1 | THR | A | 255 | −1.573 | 12.767 | 57.677 | 1.00 | 39.47 | O |
| ATOM | 674 | CG2 | THR | A | 255 | −2.105 | 11.020 | 59.253 | 1.00 | 40.24 | C |
| ATOM | 675 | N | SER | A | 256 | −1.851 | 10.258 | 54.817 | 1.00 | 40.24 | N |
| ATOM | 676 | CA | SER | A | 256 | −1.883 | 10.527 | 53.382 | 1.00 | 40.92 | C |
| ATOM | 677 | C | SER | A | 256 | −2.148 | 12.010 | 53.152 | 1.00 | 40.23 | C |
| ATOM | 678 | O | SER | A | 256 | −1.662 | 12.599 | 52.185 | 1.00 | 40.28 | O |
| ATOM | 679 | CB | SER | A | 256 | −2.968 | 9.693 | 52.690 | 1.00 | 41.53 | C |
| ATOM | 680 | OG | SER | A | 256 | −4.263 | 10.203 | 52.957 | 1.00 | 43.52 | O |
| ATOM | 681 | N | GLU | A | 257 | −2.916 | 12.610 | 54.056 | 1.00 | 39.55 | N |
| ATOM | 682 | CA | GLU | A | 257 | −3.252 | 14.024 | 53.963 | 1.00 | 38.71 | C |
| ATOM | 683 | C | GLU | A | 257 | −1.999 | 14.889 | 54.038 | 1.00 | 36.50 | C |
| ATOM | 684 | O | GLU | A | 257 | −1.825 | 15.810 | 53.240 | 1.00 | 36.20 | O |
| ATOM | 685 | CB | GLU | A | 257 | −4.221 | 14.400 | 55.085 | 1.00 | 41.43 | C |
| ATOM | 686 | CG | GLU | A | 257 | −4.650 | 15.853 | 55.090 | 1.00 | 44.63 | C |
| ATOM | 687 | CD | GLU | A | 257 | −5.747 | 16.121 | 56.103 | 1.00 | 47.39 | C |
| ATOM | 688 | OE1 | GLU | A | 257 | −6.879 | 15.634 | 55.896 | 1.00 | 48.76 | O |
| ATOM | 689 | OE2 | GLU | A | 257 | −5.476 | 16.810 | 57.109 | 1.00 | 48.99 | O |
| ATOM | 690 | N | ASP | A | 258 | −1.132 | 14.593 | 55.001 | 1.00 | 34.59 | N |
| ATOM | 691 | CA | ASP | A | 258 | 0.111 | 15.339 | 55.159 | 1.00 | 33.04 | C |
| ATOM | 692 | C | ASP | A | 258 | 1.064 | 15.047 | 54.002 | 1.00 | 32.48 | C |
| ATOM | 693 | O | ASP | A | 258 | 1.782 | 15.934 | 53.546 | 1.00 | 31.37 | O |
| ATOM | 694 | CB | ASP | A | 258 | 0.784 | 14.984 | 56.488 | 1.00 | 34.07 | C |
| ATOM | 695 | CG | ASP | A | 258 | 0.256 | 15.809 | 57.645 | 1.00 | 35.11 | C |
| ATOM | 696 | OD1 | ASP | A | 258 | 0.599 | 15.501 | 58.807 | 1.00 | 35.63 | O |
| ATOM | 697 | OD2 | ASP | A | 258 | −0.493 | 16.775 | 57.386 | 1.00 | 34.86 | O |
| ATOM | 698 | N | GLN | A | 259 | 1.072 | 13.803 | 53.532 | 1.00 | 31.90 | N |
| ATOM | 699 | CA | GLN | A | 259 | 1.940 | 13.433 | 52.417 | 1.00 | 32.81 | C |
| ATOM | 700 | C | GLN | A | 259 | 1.611 | 14.272 | 51.184 | 1.00 | 32.59 | C |
| ATOM | 701 | O | GLN | A | 259 | 2.505 | 14.820 | 50.534 | 1.00 | 32.51 | O |
| ATOM | 702 | CB | GLN | A | 259 | 1.783 | 11.946 | 52.077 | 1.00 | 32.98 | C |
| ATOM | 703 | CG | GLN | A | 259 | 2.217 | 11.000 | 53.181 | 1.00 | 34.94 | C |
| ATOM | 704 | CD | GLN | A | 259 | 2.168 | 9.547 | 52.755 | 1.00 | 37.19 | C |
| ATOM | 705 | OE1 | GLN | A | 259 | 2.322 | 8.641 | 53.576 | 1.00 | 39.55 | O |
| ATOM | 706 | NE2 | GLN | A | 259 | 1.958 | 9.315 | 51.466 | 1.00 | 37.81 | N |
| ATOM | 707 | N | ILE | A | 260 | 0.325 | 14.375 | 50.866 | 1.00 | 32.68 | N |
| ATOM | 708 | CA | ILE | A | 260 | −0.109 | 15.147 | 49.706 | 1.00 | 32.42 | C |
| ATOM | 709 | C | ILE | A | 260 | 0.183 | 16.634 | 49.880 | 1.00 | 31.57 | C |
| ATOM | 710 | O | ILE | A | 260 | 0.588 | 17.311 | 48.933 | 1.00 | 30.43 | O |
| ATOM | 711 | CB | ILE | A | 260 | −1.619 | 14.959 | 49.445 | 1.00 | 33.97 | C |
| ATOM | 712 | CG1 | ILE | A | 260 | −1.933 | 13.471 | 49.277 | 1.00 | 34.59 | C |
| ATOM | 713 | CG2 | ILE | A | 260 | −2.036 | 15.731 | 48.201 | 1.00 | 33.83 | C |
| ATOM | 714 | CD1 | ILE | A | 260 | −1.156 | 12.789 | 48.165 | 1.00 | 36.85 | C |
| ATOM | 715 | N | VAL | A | 261 | −0.029 | 17.146 | 51.088 | 1.00 | 29.87 | N |
| ATOM | 716 | CA | VAL | A | 261 | 0.244 | 18.551 | 51.358 | 1.00 | 28.91 | C |
| ATOM | 717 | C | VAL | A | 261 | 1.717 | 18.862 | 51.097 | 1.00 | 27.91 | C |
| ATOM | 718 | O | VAL | A | 261 | 2.043 | 19.856 | 50.445 | 1.00 | 28.13 | O |
| ATOM | 719 | CB | VAL | A | 261 | −0.089 | 18.923 | 52.827 | 1.00 | 28.91 | C |
| ATOM | 720 | CG1 | VAL | A | 261 | 0.472 | 20.294 | 53.161 | 1.00 | 30.09 | C |
| ATOM | 721 | CG2 | VAL | A | 261 | −1.594 | 18.911 | 53.035 | 1.00 | 31.46 | C |
| ATOM | 722 | N | LEU | A | 262 | 2.605 | 18.011 | 51.604 | 1.00 | 27.17 | N |
| ATOM | 723 | CA | LEU | A | 262 | 4.039 | 18.222 | 51.423 | 1.00 | 25.81 | C |
| ATOM | 724 | C | LEU | A | 262 | 4.461 | 18.126 | 49.955 | 1.00 | 25.46 | C |
| ATOM | 725 | O | LEU | A | 262 | 5.274 | 18.921 | 49.485 | 1.00 | 24.78 | O |
| ATOM | 726 | CB | LEU | A | 262 | 4.836 | 17.219 | 52.265 | 1.00 | 26.02 | C |
| ATOM | 727 | CG | LEU | A | 262 | 4.604 | 17.278 | 53.781 | 1.00 | 25.71 | C |
| ATOM | 728 | CD1 | LEU | A | 262 | 5.382 | 16.162 | 54.464 | 1.00 | 26.98 | C |
| ATOM | 729 | CD2 | LEU | A | 262 | 5.028 | 18.634 | 54.317 | 1.00 | 26.30 | C |
| ATOM | 730 | N | LEU | A | 263 | 3.911 | 17.155 | 49.232 | 1.00 | 25.71 | N |
| ATOM | 731 | CA | LEU | A | 263 | 4.244 | 16.985 | 47.818 | 1.00 | 26.15 | C |
| ATOM | 732 | C | LEU | A | 263 | 3.763 | 18.166 | 46.974 | 1.00 | 26.42 | C |
| ATOM | 733 | O | LEU | A | 263 | 4.514 | 18.702 | 46.154 | 1.00 | 25.91 | O |
| ATOM | 734 | CB | LEU | A | 263 | 3.633 | 15.681 | 47.283 | 1.00 | 27.20 | C |
| ATOM | 735 | CG | LEU | A | 263 | 4.293 | 14.376 | 47.745 | 1.00 | 29.34 | C |
| ATOM | 736 | CD1 | LEU | A | 263 | 3.401 | 13.197 | 47.404 | 1.00 | 30.14 | C |
| ATOM | 737 | CD2 | LEU | A | 263 | 5.658 | 14.223 | 47.082 | 1.00 | 31.00 | C |
| ATOM | 738 | N | LYS | A | 264 | 2.519 | 18.585 | 47.178 | 1.00 | 25.69 | N |
| ATOM | 739 | CA | LYS | A | 264 | 1.987 | 19.699 | 46.405 | 1.00 | 26.59 | C |
| ATOM | 740 | C | LYS | A | 264 | 2.709 | 21.011 | 46.655 | 1.00 | 26.97 | C |
| ATOM | 741 | O | LYS | A | 264 | 2.962 | 21.767 | 45.723 | 1.00 | 27.99 | O |
| ATOM | 742 | CB | LYS | A | 264 | 0.496 | 19.899 | 46.688 | 1.00 | 29.36 | C |
| ATOM | 743 | CG | LYS | A | 264 | −0.417 | 18.910 | 45.994 | 1.00 | 31.84 | C |
| ATOM | 744 | CD | LYS | A | 264 | −1.862 | 19.348 | 46.156 | 1.00 | 35.05 | C |
| ATOM | 745 | CE | LYS | A | 264 | −2.822 | 18.400 | 45.468 | 1.00 | 37.95 | C |
| ATOM | 746 | NZ | LYS | A | 264 | −4.233 | 18.872 | 45.629 | 1.00 | 39.41 | N |
| ATOM | 747 | N | SER | A | 265 | 3.047 | 21.286 | 47.908 | 1.00 | 26.64 | N |
| ATOM | 748 | CA | SER | A | 265 | 3.712 | 22.540 | 48.227 | 1.00 | 27.75 | C |
| ATOM | 749 | C | SER | A | 265 | 5.199 | 22.591 | 47.884 | 1.00 | 26.92 | C |
| ATOM | 750 | O | SER | A | 265 | 5.750 | 23.676 | 47.723 | 1.00 | 28.28 | O |
| ATOM | 751 | CB | SER | A | 265 | 3.513 | 22.881 | 49.709 | 1.00 | 28.81 | C |
| ATOM | 752 | OG | SER | A | 265 | 4.101 | 21.902 | 50.540 | 1.00 | 33.64 | O |
| ATOM | 753 | N | SER | A | 266 | 5.847 | 21.434 | 47.757 | 1.00 | 25.17 | N |
| ATOM | 754 | CA | SER | A | 266 | 7.277 | 21.412 | 47.449 | 1.00 | 23.91 | C |
| ATOM | 755 | C | SER | A | 266 | 7.609 | 21.035 | 46.011 | 1.00 | 23.51 | C |
| ATOM | 756 | O | SER | A | 266 | 8.749 | 21.206 | 45.572 | 1.00 | 23.30 | O |
| ATOM | 757 | CB | SER | A | 266 | 8.001 | 20.445 | 48.385 | 1.00 | 24.45 | C |
| ATOM | 758 | OG | SER | A | 266 | 7.656 | 19.101 | 48.094 | 1.00 | 24.60 | O |
| ATOM | 759 | N | ALA | A | 267 | 6.619 | 20.519 | 45.285 | 1.00 | 22.67 | N |
| ATOM | 760 | CA | ALA | A | 267 | 6.801 | 20.089 | 43.898 | 1.00 | 23.39 | C |
| ATOM | 761 | C | ALA | A | 267 | 7.698 | 20.979 | 43.040 | 1.00 | 23.51 | C |
| ATOM | 762 | O | ALA | A | 267 | 8.716 | 20.517 | 42.515 | 1.00 | 23.55 | O |
| ATOM | 763 | CB | ALA | A | 267 | 5.436 | 19.938 | 43.217 | 1.00 | 24.51 | C |
| ATOM | 764 | N | ILE | A | 268 | 7.330 | 22.247 | 42.883 | 1.00 | 22.01 | N |
| ATOM | 765 | CA | ILE | A | 268 | 8.132 | 23.135 | 42.041 | 1.00 | 22.23 | C |
| ATOM | 766 | C | ILE | A | 268 | 9.539 | 23.374 | 42.592 | 1.00 | 22.05 | C |
| ATOM | 767 | O | ILE | A | 268 | 10.494 | 23.558 | 41.828 | 1.00 | 20.90 | O |
| ATOM | 768 | CB | ILE | A | 268 | 7.426 | 24.496 | 41.811 | 1.00 | 23.63 | C |
| ATOM | 769 | CG1 | ILE | A | 268 | 8.097 | 25.232 | 40.645 | 1.00 | 24.85 | C |
| ATOM | 770 | CG2 | ILE | A | 268 | 7.484 | 25.354 | 43.068 | 1.00 | 25.18 | C |
| ATOM | 771 | CD1 | ILE | A | 268 | 7.933 | 24.549 | 39.303 | 1.00 | 25.69 | C |
| ATOM | 772 | N | GLU | A | 269 | 9.674 | 23.352 | 43.911 | 1.00 | 20.40 | N |
| ATOM | 773 | CA | GLU | A | 269 | 10.979 | 23.561 | 44.529 | 1.00 | 20.63 | C |
| ATOM | 774 | C | GLU | A | 269 | 11.933 | 22.402 | 44.268 | 1.00 | 21.33 | C |
| ATOM | 775 | O | GLU | A | 269 | 13.109 | 22.620 | 43.976 | 1.00 | 20.99 | O |
| ATOM | 776 | CB | GLU | A | 269 | 10.823 | 23.770 | 46.030 | 1.00 | 20.38 | C |
| ATOM | 777 | CG | GLU | A | 269 | 10.206 | 25.110 | 46.396 | 1.00 | 22.10 | C |
| ATOM | 778 | CD | GLU | A | 269 | 10.009 | 25.261 | 47.892 | 1.00 | 23.72 | C |
| ATOM | 779 | OE1 | GLU | A | 269 | 10.803 | 24.670 | 48.656 | 1.00 | 22.73 | O |
| ATOM | 780 | OE2 | GLU | A | 269 | 9.067 | 25.974 | 48.301 | 1.00 | 24.58 | O |
| ATOM | 781 | N | VAL | A | 270 | 11.434 | 21.172 | 44.375 | 1.00 | 20.39 | N |
| ATOM | 782 | CA | VAL | A | 270 | 12.279 | 20.006 | 44.143 | 1.00 | 20.83 | C |
| ATOM | 783 | C | VAL | A | 270 | 12.644 | 19.911 | 42.670 | 1.00 | 20.52 | C |
| ATOM | 784 | O | VAL | A | 270 | 13.734 | 19.458 | 42.318 | 1.00 | 20.87 | O |
| ATOM | 785 | CB | VAL | A | 270 | 11.582 | 18.709 | 44.597 | 1.00 | 21.55 | C |
| ATOM | 786 | CG1 | VAL | A | 270 | 12.481 | 17.512 | 44.318 | 1.00 | 21.95 | C |
| ATOM | 787 | CG2 | VAL | A | 270 | 11.268 | 18.790 | 46.086 | 1.00 | 23.25 | C |
| ATOM | 788 | N | ILE | A | 271 | 11.731 | 20.337 | 41.804 | 1.00 | 20.29 | N |
| ATOM | 789 | CA | ILE | A | 271 | 12.010 | 20.318 | 40.376 | 1.00 | 20.71 | C |
| ATOM | 790 | C | ILE | A | 271 | 13.145 | 21.300 | 40.099 | 1.00 | 20.86 | C |
| ATOM | 791 | O | ILE | A | 271 | 14.083 | 20.990 | 39.361 | 1.00 | 20.78 | O |
| ATOM | 792 | CB | ILE | A | 271 | 10.755 | 20.684 | 39.563 | 1.00 | 21.89 | C |
| ATOM | 793 | CG1 | ILE | A | 271 | 9.842 | 19.450 | 39.483 | 1.00 | 24.21 | C |
| ATOM | 794 | CG2 | ILE | A | 271 | 11.149 | 21.173 | 38.170 | 1.00 | 23.03 | C |
| ATOM | 795 | CD1 | ILE | A | 271 | 8.489 | 19.711 | 38.852 | 1.00 | 27.85 | C |
| ATOM | 796 | N | MET | A | 272 | 13.076 | 22.481 | 40.701 | 1.00 | 21.17 | N |
| ATOM | 797 | CA | MET | A | 272 | 14.147 | 23.446 | 40.500 | 1.00 | 21.57 | C |
| ATOM | 798 | C | MET | A | 272 | 15.474 | 22.888 | 41.020 | 1.00 | 20.82 | C |
| ATOM | 799 | O | MET | A | 272 | 16.513 | 23.064 | 40.384 | 1.00 | 22.20 | O |
| ATOM | 800 | CB | MET | A | 272 | 13.800 | 24.770 | 41.183 | 1.00 | 22.31 | C |
| ATOM | 801 | CG | MET | A | 272 | 12.595 | 25.441 | 40.549 | 1.00 | 24.16 | C |
| ATOM | 802 | SD | MET | A | 272 | 12.222 | 27.036 | 41.296 | 1.00 | 26.22 | S |
| ATOM | 803 | CE | MET | A | 272 | 11.003 | 27.687 | 40.134 | 1.00 | 26.38 | C |
| ATOM | 804 | N | LEU | A | 273 | 15.442 | 22.204 | 42.163 | 1.00 | 21.17 | N |
| ATOM | 805 | CA | LEU | A | 273 | 16.661 | 21.606 | 42.717 | 1.00 | 21.28 | C |
| ATOM | 806 | C | LEU | A | 273 | 17.226 | 20.486 | 41.842 | 1.00 | 20.96 | C |
| ATOM | 807 | O | LEU | A | 273 | 18.408 | 20.494 | 41.487 | 1.00 | 20.75 | O |
| ATOM | 808 | CB | LEU | A | 273 | 16.405 | 21.026 | 44.116 | 1.00 | 22.98 | C |
| ATOM | 809 | CG | LEU | A | 273 | 16.367 | 21.940 | 45.337 | 1.00 | 25.62 | C |
| ATOM | 810 | CD1 | LEU | A | 273 | 15.959 | 21.129 | 46.572 | 1.00 | 25.83 | C |
| ATOM | 811 | CD2 | LEU | A | 273 | 17.736 | 22.571 | 45.543 | 1.00 | 26.65 | C |
| ATOM | 812 | N | ARG | A | 274 | 16.385 | 19.517 | 41.494 | 1.00 | 19.69 | N |
| ATOM | 813 | CA | ARG | A | 274 | 16.852 | 18.384 | 40.702 | 1.00 | 19.52 | C |
| ATOM | 814 | C | ARG | A | 274 | 17.317 | 18.787 | 39.309 | 1.00 | 19.10 | C |
| ATOM | 815 | O | ARG | A | 274 | 18.159 | 18.117 | 38.715 | 1.00 | 19.83 | O |
| ATOM | 816 | CB | ARG | A | 274 | 15.759 | 17.299 | 40.610 | 1.00 | 19.75 | C |
| ATOM | 817 | CG | ARG | A | 274 | 14.652 | 17.566 | 39.601 | 1.00 | 19.52 | C |
| ATOM | 818 | CD | ARG | A | 274 | 13.381 | 16.792 | 39.969 | 1.00 | 19.72 | C |
| ATOM | 819 | NE | ARG | A | 274 | 13.599 | 15.356 | 40.153 | 1.00 | 18.11 | N |
| ATOM | 820 | CZ | ARG | A | 274 | 13.580 | 14.453 | 39.175 | 1.00 | 19.01 | C |
| ATOM | 821 | NH1 | ARG | A | 274 | 13.357 | 14.824 | 37.919 | 1.00 | 18.53 | N |
| ATOM | 822 | NH2 | ARG | A | 274 | 13.759 | 13.168 | 39.458 | 1.00 | 19.51 | N |
| ATOM | 823 | N | SER | A | 275 | 16.792 | 19.892 | 38.793 | 1.00 | 19.73 | N |
| ATOM | 824 | CA | SER | A | 275 | 17.183 | 20.331 | 37.463 | 1.00 | 19.93 | C |
| ATOM | 825 | C | SER | A | 275 | 18.615 | 20.838 | 37.442 | 1.00 | 19.90 | C |
| ATOM | 826 | O | SER | A | 275 | 19.191 | 21.016 | 36.377 | 1.00 | 20.21 | O |
| ATOM | 827 | CB | SER | A | 275 | 16.249 | 21.437 | 36.958 | 1.00 | 20.51 | C |
| ATOM | 828 | OG | SER | A | 275 | 16.520 | 22.680 | 37.579 | 1.00 | 20.38 | O |
| ATOM | 829 | N | ASN | A | 276 | 19.198 | 21.055 | 38.615 | 1.00 | 20.28 | N |
| ATOM | 830 | CA | ASN | A | 276 | 20.564 | 21.557 | 38.662 | 1.00 | 19.85 | C |
| ATOM | 831 | C | ASN | A | 276 | 21.512 | 20.544 | 38.024 | 1.00 | 21.26 | C |
| ATOM | 832 | O | ASN | A | 276 | 22.585 | 20.903 | 37.538 | 1.00 | 19.72 | O |
| ATOM | 833 | CB | ASN | A | 276 | 20.983 | 21.843 | 40.108 | 1.00 | 20.77 | C |
| ATOM | 834 | CG | ASN | A | 276 | 22.265 | 22.651 | 40.187 | 1.00 | 23.39 | C |
| ATOM | 835 | OD1 | ASN | A | 276 | 23.275 | 22.187 | 40.713 | 1.00 | 26.18 | O |
| ATOM | 836 | ND2 | ASN | A | 276 | 22.231 | 23.867 | 39.649 | 1.00 | 21.92 | N |
| ATOM | 837 | N | GLU | A | 277 | 21.096 | 19.280 | 38.000 | 1.00 | 20.52 | N |
| ATOM | 838 | CA | GLU | A | 277 | 21.925 | 18.226 | 37.425 | 1.00 | 21.75 | C |
| ATOM | 839 | C | GLU | A | 277 | 22.103 | 18.370 | 35.908 | 1.00 | 21.79 | C |
| ATOM | 840 | O | GLU | A | 277 | 23.105 | 17.910 | 35.351 | 1.00 | 22.41 | O |
| ATOM | 841 | CB | GLU | A | 277 | 21.331 | 16.852 | 37.785 | 1.00 | 22.91 | C |
| ATOM | 842 | CG | GLU | A | 277 | 22.199 | 15.659 | 37.413 | 1.00 | 26.24 | C |
| ATOM | 843 | CD | GLU | A | 277 | 21.904 | 14.418 | 38.261 | 1.00 | 28.07 | C |
| ATOM | 844 | OE1 | GLU | A | 277 | 22.359 | 13.319 | 37.875 | 1.00 | 30.43 | O |
| ATOM | 845 | OE2 | GLU | A | 277 | 21.233 | 14.532 | 39.317 | 1.00 | 26.56 | O |
| ATOM | 846 | N | SER | A | 278 | 21.152 | 19.011 | 35.234 | 1.00 | 19.68 | N |
| ATOM | 847 | CA | SER | A | 278 | 21.266 | 19.194 | 33.789 | 1.00 | 20.64 | C |
| ATOM | 848 | C | SER | A | 278 | 21.712 | 20.607 | 33.448 | 1.00 | 21.58 | C |
| ATOM | 849 | O | SER | A | 278 | 22.008 | 20.910 | 32.292 | 1.00 | 22.05 | O |
| ATOM | 850 | CB | SER | A | 278 | 19.934 | 18.910 | 33.092 | 1.00 | 20.93 | C |
| ATOM | 851 | OG | SER | A | 278 | 18.941 | 19.829 | 33.497 | 1.00 | 22.00 | O |
| ATOM | 852 | N | PHE | A | 279 | 21.751 | 21.474 | 34.451 | 1.00 | 21.92 | N |
| ATOM | 853 | CA | PHE | A | 279 | 22.160 | 22.853 | 34.219 | 1.00 | 23.24 | C |
| ATOM | 854 | C | PHE | A | 279 | 23.659 | 22.912 | 33.972 | 1.00 | 24.55 | C |
| ATOM | 855 | O | PHE | A | 279 | 24.429 | 22.218 | 34.638 | 1.00 | 24.49 | O |
| ATOM | 856 | CB | PHE | A | 279 | 21.820 | 23.723 | 35.429 | 1.00 | 23.08 | C |
| ATOM | 857 | CG | PHE | A | 279 | 22.051 | 25.187 | 35.198 | 1.00 | 24.02 | C |
| ATOM | 858 | CD1 | PHE | A | 279 | 21.135 | 25.942 | 34.471 | 1.00 | 24.96 | C |
| ATOM | 859 | CD2 | PHE | A | 279 | 23.197 | 25.805 | 35.682 | 1.00 | 24.94 | C |
| ATOM | 860 | CE1 | PHE | A | 279 | 21.356 | 27.293 | 34.227 | 1.00 | 24.93 | C |
| ATOM | 861 | CE2 | PHE | A | 279 | 23.429 | 27.160 | 35.442 | 1.00 | 25.50 | C |
| ATOM | 862 | CZ | PHE | A | 279 | 22.506 | 27.903 | 34.714 | 1.00 | 24.47 | C |
| ATOM | 863 | N | THR | A | 280 | 24.077 | 23.728 | 33.010 | 1.00 | 24.73 | N |
| ATOM | 864 | CA | THR | A | 280 | 25.496 | 23.872 | 32.728 | 1.00 | 26.87 | C |
| ATOM | 865 | C | THR | A | 280 | 25.884 | 25.343 | 32.672 | 1.00 | 27.44 | C |
| ATOM | 866 | O | THR | A | 280 | 25.186 | 26.162 | 32.070 | 1.00 | 26.28 | O |
| ATOM | 867 | CB | THR | A | 280 | 25.897 | 23.198 | 31.399 | 1.00 | 27.76 | C |
| ATOM | 868 | OG1 | THR | A | 280 | 27.298 | 23.408 | 31.173 | 1.00 | 31.72 | O |
| ATOM | 869 | CG2 | THR | A | 280 | 25.107 | 23.768 | 30.236 | 1.00 | 27.79 | C |
| ATOM | 870 | N | MET | A | 281 | 26.991 | 25.676 | 33.326 | 1.00 | 28.33 | N |
| ATOM | 871 | CA | MET | A | 281 | 27.469 | 27.049 | 33.340 | 1.00 | 31.03 | C |
| ATOM | 872 | C | MET | A | 281 | 28.275 | 27.390 | 32.095 | 1.00 | 31.28 | C |
| ATOM | 873 | O | MET | A | 281 | 28.812 | 28.490 | 31.980 | 1.00 | 30.87 | O |
| ATOM | 874 | CB | MET | A | 281 | 28.298 | 27.306 | 34.596 | 1.00 | 33.43 | C |
| ATOM | 875 | CG | MET | A | 281 | 27.448 | 27.518 | 35.835 | 1.00 | 36.11 | C |
| ATOM | 876 | SD | MET | A | 281 | 28.429 | 27.829 | 37.295 | 1.00 | 39.85 | S |
| ATOM | 877 | CE | MET | A | 281 | 28.995 | 29.495 | 36.967 | 1.00 | 40.40 | C |
| ATOM | 878 | N | ASP | A | 282 | 28.364 | 26.448 | 31.159 | 1.00 | 31.72 | N |
| ATOM | 879 | CA | ASP | A | 282 | 29.097 | 26.709 | 29.925 | 1.00 | 32.91 | C |
| ATOM | 880 | C | ASP | A | 282 | 28.366 | 27.818 | 29.175 | 1.00 | 32.02 | C |
| ATOM | 881 | O | ASP | A | 282 | 28.989 | 28.764 | 28.683 | 1.00 | 31.15 | O |
| ATOM | 882 | CB | ASP | A | 282 | 29.172 | 25.455 | 29.050 | 1.00 | 35.93 | C |
| ATOM | 883 | CG | ASP | A | 282 | 29.947 | 24.328 | 29.708 | 1.00 | 39.91 | C |
| ATOM | 884 | OD1 | ASP | A | 282 | 30.940 | 24.619 | 30.412 | 1.00 | 42.35 | O |
| ATOM | 885 | OD2 | ASP | A | 282 | 29.573 | 23.150 | 29.508 | 1.00 | 42.45 | O |
| ATOM | 886 | N | ASP | A | 283 | 27.041 | 27.702 | 29.100 | 1.00 | 29.87 | N |
| ATOM | 887 | CA | ASP | A | 283 | 26.224 | 28.704 | 28.418 | 1.00 | 28.59 | C |
| ATOM | 888 | C | ASP | A | 283 | 24.931 | 29.032 | 29.170 | 1.00 | 27.92 | C |
| ATOM | 889 | O | ASP | A | 283 | 23.984 | 29.568 | 28.592 | 1.00 | 27.21 | O |
| ATOM | 890 | CB | ASP | A | 283 | 25.904 | 28.243 | 26.994 | 1.00 | 29.84 | C |
| ATOM | 891 | CG | ASP | A | 283 | 25.030 | 27.006 | 26.958 | 1.00 | 31.11 | C |
| ATOM | 892 | OD1 | ASP | A | 283 | 24.872 | 26.351 | 28.009 | 1.00 | 28.99 | O |
| ATOM | 893 | OD2 | ASP | A | 283 | 24.507 | 26.687 | 25.870 | 1.00 | 32.79 | O |
| ATOM | 894 | N | MET | A | 284 | 24.902 | 28.708 | 30.460 | 1.00 | 26.84 | N |
| ATOM | 895 | CA | MET | A | 284 | 23.748 | 28.985 | 31.317 | 1.00 | 27.62 | C |
| ATOM | 896 | C | MET | A | 284 | 22.449 | 28.379 | 30.801 | 1.00 | 27.20 | C |
| ATOM | 897 | O | MET | A | 284 | 21.429 | 29.060 | 30.686 | 1.00 | 27.86 | O |
| ATOM | 898 | CB | MET | A | 284 | 23.565 | 30.497 | 31.484 | 1.00 | 29.95 | C |
| ATOM | 899 | CG | MET | A | 284 | 24.785 | 31.219 | 32.031 | 1.00 | 33.34 | C |
| ATOM | 900 | SD | MET | A | 284 | 25.323 | 30.578 | 33.624 | 1.00 | 36.23 | S |
| ATOM | 901 | CE | MET | A | 284 | 26.985 | 31.242 | 33.719 | 1.00 | 35.78 | C |
| ATOM | 902 | N | SER | A | 285 | 22.479 | 27.091 | 30.503 | 1.00 | 25.58 | N |
| ATOM | 903 | CA | SER | A | 285 | 21.288 | 26.427 | 30.010 | 1.00 | 24.62 | C |
| ATOM | 904 | C | SER | A | 285 | 21.136 | 25.090 | 30.697 | 1.00 | 24.72 | C |
| ATOM | 905 | O | SER | A | 285 | 22.028 | 24.641 | 31.415 | 1.00 | 24.18 | O |
| ATOM | 906 | CB | SER | A | 285 | 21.402 | 26.186 | 28.509 | 1.00 | 24.98 | C |
| ATOM | 907 | OG | SER | A | 285 | 22.415 | 25.224 | 28.241 | 1.00 | 25.94 | O |
| ATOM | 908 | N | TRP | A | 286 | 19.982 | 24.472 | 30.480 | 1.00 | 24.17 | N |
| ATOM | 909 | CA | TRP | A | 286 | 19.699 | 23.146 | 30.997 | 1.00 | 24.74 | C |
| ATOM | 910 | C | TRP | A | 286 | 19.842 | 22.312 | 29.732 | 1.00 | 25.34 | C |
| ATOM | 911 | O | TRP | A | 286 | 19.006 | 22.391 | 28.828 | 1.00 | 25.37 | O |
| ATOM | 912 | CB | TRP | A | 286 | 18.268 | 23.064 | 31.522 | 1.00 | 23.76 | C |
| ATOM | 913 | CG | TRP | A | 286 | 18.048 | 23.702 | 32.863 | 1.00 | 21.76 | C |
| ATOM | 914 | CD1 | TRP | A | 286 | 18.186 | 23.107 | 34.088 | 1.00 | 21.47 | C |
| ATOM | 915 | CD2 | TRP | A | 286 | 17.568 | 25.031 | 33.118 | 1.00 | 23.03 | C |
| ATOM | 916 | NE1 | TRP | A | 286 | 17.811 | 23.976 | 35.084 | 1.00 | 21.88 | N |
| ATOM | 917 | CE2 | TRP | A | 286 | 17.429 | 25.164 | 34.519 | 1.00 | 22.96 | C |
| ATOM | 918 | CE3 | TRP | A | 286 | 17.238 | 26.121 | 32.299 | 1.00 | 23.54 | C |
| ATOM | 919 | CZ2 | TRP | A | 286 | 16.970 | 26.341 | 35.120 | 1.00 | 24.15 | C |
| ATOM | 920 | CZ3 | TRP | A | 286 | 16.781 | 27.293 | 32.898 | 1.00 | 22.92 | C |
| ATOM | 921 | CH2 | TRP | A | 286 | 16.651 | 27.390 | 34.297 | 1.00 | 23.66 | C |
| ATOM | 922 | N | THR | A | 287 | 20.918 | 21.540 | 29.654 | 1.00 | 25.53 | N |
| ATOM | 923 | CA | THR | A | 287 | 21.173 | 20.721 | 28.478 | 1.00 | 27.14 | C |
| ATOM | 924 | C | THR | A | 287 | 20.833 | 19.266 | 28.753 | 1.00 | 27.53 | C |
| ATOM | 925 | O | THR | A | 287 | 21.501 | 18.607 | 29.551 | 1.00 | 27.40 | O |
| ATOM | 926 | CB | THR | A | 287 | 22.644 | 20.853 | 28.049 | 1.00 | 27.77 | C |
| ATOM | 927 | OG1 | THR | A | 287 | 22.914 | 22.229 | 27.733 | 1.00 | 30.32 | O |
| ATOM | 928 | CG2 | THR | A | 287 | 22.922 | 20.000 | 26.824 | 1.00 | 29.69 | C |
| ATOM | 929 | N | CYS | A | 288 | 19.792 | 18.775 | 28.084 | 1.00 | 28.08 | N |
| ATOM | 930 | CA | CYS | A | 288 | 19.326 | 17.406 | 28.270 | 1.00 | 30.34 | C |
| ATOM | 931 | C | CYS | A | 288 | 19.478 | 16.520 | 27.040 | 1.00 | 33.66 | C |
| ATOM | 932 | O | CYS | A | 288 | 18.530 | 15.857 | 26.624 | 1.00 | 33.19 | O |
| ATOM | 933 | CB | CYS | A | 288 | 17.861 | 17.426 | 28.699 | 1.00 | 29.32 | C |
| ATOM | 934 | SG | CYS | A | 288 | 17.566 | 18.403 | 30.188 | 1.00 | 28.01 | S |
| ATOM | 935 | N | GLY | A | 289 | 20.675 | 16.498 | 26.466 | 1.00 | 37.69 | N |
| ATOM | 936 | CA | GLY | A | 289 | 20.897 | 15.682 | 25.286 | 1.00 | 41.85 | C |
| ATOM | 937 | C | GLY | A | 289 | 21.072 | 16.536 | 24.044 | 1.00 | 44.11 | C |
| ATOM | 938 | O | GLY | A | 289 | 21.842 | 17.497 | 24.051 | 1.00 | 45.10 | O |
| ATOM | 939 | N | ASN | A | 290 | 20.349 | 16.205 | 22.978 | 1.00 | 46.33 | N |
| ATOM | 940 | CA | ASN | A | 290 | 20.469 | 16.959 | 21.737 | 1.00 | 47.32 | C |
| ATOM | 941 | C | ASN | A | 290 | 19.961 | 18.391 | 21.874 | 1.00 | 47.22 | C |
| ATOM | 942 | O | ASN | A | 290 | 19.303 | 18.746 | 22.857 | 1.00 | 47.49 | O |
| ATOM | 943 | CB | ASN | A | 290 | 19.733 | 16.241 | 20.600 | 1.00 | 49.56 | C |
| ATOM | 944 | CG | ASN | A | 290 | 18.235 | 16.224 | 20.792 | 1.00 | 51.07 | C |
| ATOM | 945 | OD1 | ASN | A | 290 | 17.591 | 17.271 | 20.803 | 1.00 | 52.29 | O |
| ATOM | 946 | ND2 | ASN | A | 290 | 17.668 | 15.032 | 20.944 | 1.00 | 51.45 | N |
| ATOM | 947 | N | GLN | A | 291 | 20.277 | 19.205 | 20.874 | 1.00 | 46.24 | N |
| ATOM | 948 | CA | GLN | A | 291 | 19.896 | 20.611 | 20.850 | 1.00 | 45.60 | C |
| ATOM | 949 | C | GLN | A | 291 | 18.402 | 20.859 | 21.031 | 1.00 | 43.20 | C |
| ATOM | 950 | O | GLN | A | 291 | 18.007 | 21.916 | 21.520 | 1.00 | 43.27 | O |
| ATOM | 951 | CB | GLN | A | 291 | 20.380 | 21.247 | 19.545 | 1.00 | 47.46 | C |
| ATOM | 952 | CG | GLN | A | 291 | 21.879 | 21.087 | 19.325 | 1.00 | 50.94 | C |
| ATOM | 953 | CD | GLN | A | 291 | 22.705 | 21.786 | 20.395 | 1.00 | 52.59 | C |
| ATOM | 954 | OE1 | GLN | A | 291 | 23.893 | 21.503 | 20.563 | 1.00 | 54.12 | O |
| ATOM | 955 | NE2 | GLN | A | 291 | 22.081 | 22.712 | 21.114 | 1.00 | 53.69 | N |
| ATOM | 956 | N | ASP | A | 292 | 17.574 | 19.897 | 20.636 | 1.00 | 40.92 | N |
| ATOM | 957 | CA | ASP | A | 292 | 16.129 | 20.046 | 20.780 | 1.00 | 38.58 | C |
| ATOM | 958 | C | ASP | A | 292 | 15.740 | 20.140 | 22.252 | 1.00 | 35.80 | C |
| ATOM | 959 | O | ASP | A | 292 | 14.769 | 20.814 | 22.601 | 1.00 | 34.04 | O |
| ATOM | 960 | CB | ASP | A | 292 | 15.391 | 18.862 | 20.145 | 1.00 | 41.69 | C |
| ATOM | 961 | CG | ASP | A | 292 | 15.325 | 18.950 | 18.629 | 1.00 | 44.13 | C |
| ATOM | 962 | OD1 | ASP | A | 292 | 14.862 | 17.973 | 18.002 | 1.00 | 45.48 | O |
| ATOM | 963 | OD2 | ASP | A | 292 | 15.724 | 19.993 | 18.067 | 1.00 | 45.78 | O |
| ATOM | 964 | N | TYR | A | 293 | 16.506 | 19.469 | 23.111 | 1.00 | 33.09 | N |
| ATOM | 965 | CA | TYR | A | 293 | 16.219 | 19.465 | 24.543 | 1.00 | 31.43 | C |
| ATOM | 966 | C | TYR | A | 293 | 17.183 | 20.305 | 25.367 | 1.00 | 30.28 | C |
| ATOM | 967 | O | TYR | A | 293 | 17.558 | 19.934 | 26.481 | 1.00 | 30.56 | O |
| ATOM | 968 | CB | TYR | A | 293 | 16.186 | 18.027 | 25.066 | 1.00 | 31.64 | C |
| ATOM | 969 | CG | TYR | A | 293 | 15.232 | 17.154 | 24.287 | 1.00 | 31.43 | C |
| ATOM | 970 | CD1 | TYR | A | 293 | 15.591 | 15.864 | 23.905 | 1.00 | 32.22 | C |
| ATOM | 971 | CD2 | TYR | A | 293 | 13.999 | 17.647 | 23.861 | 1.00 | 32.09 | C |
| ATOM | 972 | CE1 | TYR | A | 293 | 14.752 | 15.091 | 23.106 | 1.00 | 32.84 | C |
| ATOM | 973 | CE2 | TYR | A | 293 | 13.153 | 16.883 | 23.063 | 1.00 | 31.78 | C |
| ATOM | 974 | CZ | TYR | A | 293 | 13.537 | 15.611 | 22.684 | 1.00 | 33.14 | C |
| ATOM | 975 | OH | TYR | A | 293 | 12.726 | 14.874 | 21.850 | 1.00 | 32.75 | O |
| ATOM | 976 | N | LYS | A | 294 | 17.594 | 21.431 | 24.801 | 1.00 | 29.44 | N |
| ATOM | 977 | CA | LYS | A | 294 | 18.466 | 22.369 | 25.494 | 1.00 | 27.92 | C |
| ATOM | 978 | C | LYS | A | 294 | 17.529 | 23.530 | 25.786 | 1.00 | 27.57 | C |
| ATOM | 979 | O | LYS | A | 294 | 16.947 | 24.114 | 24.866 | 1.00 | 27.85 | O |
| ATOM | 980 | CB | LYS | A | 294 | 19.618 | 22.833 | 24.595 | 1.00 | 31.41 | C |
| ATOM | 981 | CG | LYS | A | 294 | 20.500 | 23.907 | 25.239 | 1.00 | 32.77 | C |
| ATOM | 982 | CD | LYS | A | 294 | 21.578 | 24.416 | 24.284 | 1.00 | 36.06 | C |
| ATOM | 983 | CE | LYS | A | 294 | 22.872 | 23.633 | 24.419 | 1.00 | 37.32 | C |
| ATOM | 984 | NZ | LYS | A | 294 | 23.599 | 23.990 | 25.673 | 1.00 | 37.49 | N |
| ATOM | 985 | N | TYR | A | 295 | 17.363 | 23.852 | 27.061 | 1.00 | 25.07 | N |
| ATOM | 986 | CA | TYR | A | 295 | 16.465 | 24.928 | 27.451 | 1.00 | 24.97 | C |
| ATOM | 987 | C | TYR | A | 295 | 17.208 | 26.154 | 27.938 | 1.00 | 25.69 | C |
| ATOM | 988 | O | TYR | A | 295 | 18.005 | 26.074 | 28.865 | 1.00 | 24.37 | O |
| ATOM | 989 | CB | TYR | A | 295 | 15.517 | 24.431 | 28.543 | 1.00 | 24.19 | C |
| ATOM | 990 | CG | TYR | A | 295 | 14.927 | 23.080 | 28.216 | 1.00 | 24.03 | C |
| ATOM | 991 | CD1 | TYR | A | 295 | 15.297 | 21.943 | 28.932 | 1.00 | 23.33 | C |
| ATOM | 992 | CD2 | TYR | A | 295 | 14.023 | 22.933 | 27.167 | 1.00 | 23.84 | C |
| ATOM | 993 | CE1 | TYR | A | 295 | 14.780 | 20.692 | 28.611 | 1.00 | 24.85 | C |
| ATOM | 994 | CE2 | TYR | A | 295 | 13.500 | 21.688 | 26.836 | 1.00 | 24.18 | C |
| ATOM | 995 | CZ | TYR | A | 295 | 13.882 | 20.573 | 27.563 | 1.00 | 24.74 | C |
| ATOM | 996 | OH | TYR | A | 295 | 13.369 | 19.338 | 27.244 | 1.00 | 24.72 | O |
| ATOM | 997 | N | ARG | A | 296 | 16.921 | 27.286 | 27.302 | 1.00 | 27.37 | N |
| ATOM | 998 | CA | ARG | A | 296 | 17.532 | 28.566 | 27.632 | 1.00 | 29.21 | C |
| ATOM | 999 | C | ARG | A | 296 | 16.457 | 29.505 | 28.177 | 1.00 | 28.74 | C |
| ATOM | 1000 | O | ARG | A | 296 | 15.269 | 29.177 | 28.171 | 1.00 | 28.04 | O |
| ATOM | 1001 | CB | ARG | A | 296 | 18.140 | 29.201 | 26.377 | 1.00 | 31.67 | C |
| ATOM | 1002 | CG | ARG | A | 296 | 19.115 | 28.332 | 25.590 | 1.00 | 36.71 | C |
| ATOM | 1003 | CD | ARG | A | 296 | 19.581 | 29.091 | 24.352 | 1.00 | 40.42 | C |
| ATOM | 1004 | NE | ARG | A | 296 | 20.676 | 28.444 | 23.631 | 1.00 | 44.23 | N |
| ATOM | 1005 | CZ | ARG | A | 296 | 20.533 | 27.442 | 22.769 | 1.00 | 46.02 | C |
| ATOM | 1006 | NH1 | ARG | A | 296 | 19.329 | 26.949 | 22.508 | 1.00 | 46.77 | N |
| ATOM | 1007 | NH2 | ARG | A | 296 | 21.597 | 26.941 | 22.152 | 1.00 | 46.52 | N |
| ATOM | 1008 | N | VAL | A | 297 | 16.879 | 30.678 | 28.634 | 1.00 | 29.11 | N |
| ATOM | 1009 | CA | VAL | A | 297 | 15.956 | 31.675 | 29.167 | 1.00 | 30.41 | C |
| ATOM | 1010 | C | VAL | A | 297 | 14.821 | 31.972 | 28.187 | 1.00 | 30.24 | C |
| ATOM | 1011 | O | VAL | A | 297 | 13.655 | 32.065 | 28.582 | 1.00 | 29.94 | O |
| ATOM | 1012 | CB | VAL | A | 297 | 16.692 | 33.005 | 29.475 | 1.00 | 30.71 | C |
| ATOM | 1013 | CG1 | VAL | A | 297 | 15.686 | 34.103 | 29.797 | 1.00 | 33.35 | C |
| ATOM | 1014 | CG2 | VAL | A | 297 | 17.646 | 32.811 | 30.644 | 1.00 | 32.09 | C |
| ATOM | 1015 | N | SER | A | 298 | 15.168 | 32.115 | 26.912 | 1.00 | 30.44 | N |
| ATOM | 1016 | CA | SER | A | 298 | 14.185 | 32.430 | 25.881 | 1.00 | 30.65 | C |
| ATOM | 1017 | C | SER | A | 298 | 13.106 | 31.370 | 25.714 | 1.00 | 30.99 | C |
| ATOM | 1018 | O | SER | A | 298 | 11.986 | 31.680 | 25.304 | 1.00 | 31.34 | O |
| ATOM | 1019 | CB | SER | A | 298 | 14.884 | 32.675 | 24.539 | 1.00 | 31.86 | C |
| ATOM | 1020 | OG | SER | A | 298 | 15.658 | 31.559 | 24.143 | 1.00 | 33.35 | O |
| ATOM | 1021 | N | ASP | A | 299 | 13.435 | 30.121 | 26.028 | 1.00 | 29.88 | N |
| ATOM | 1022 | CA | ASP | A | 299 | 12.464 | 29.042 | 25.912 | 1.00 | 29.41 | C |
| ATOM | 1023 | C | ASP | A | 299 | 11.424 | 29.137 | 27.019 | 1.00 | 28.20 | C |
| ATOM | 1024 | O | ASP | A | 299 | 10.268 | 28.770 | 26.827 | 1.00 | 28.75 | O |
| ATOM | 1025 | CB | ASP | A | 299 | 13.162 | 27.679 | 25.979 | 1.00 | 31.09 | C |
| ATOM | 1026 | CG | ASP | A | 299 | 14.070 | 27.435 | 24.797 | 1.00 | 34.22 | C |
| ATOM | 1027 | OD1 | ASP | A | 299 | 13.589 | 27.548 | 23.651 | 1.00 | 34.74 | O |
| ATOM | 1028 | OD2 | ASP | A | 299 | 15.263 | 27.129 | 25.013 | 1.00 | 36.25 | O |
| ATOM | 1029 | N | VAL | A | 300 | 11.837 | 29.631 | 28.183 | 1.00 | 27.61 | N |
| ATOM | 1030 | CA | VAL | A | 300 | 10.923 | 29.760 | 29.308 | 1.00 | 26.53 | C |
| ATOM | 1031 | C | VAL | A | 300 | 9.948 | 30.913 | 29.070 | 1.00 | 26.97 | C |
| ATOM | 1032 | O | VAL | A | 300 | 8.781 | 30.835 | 29.449 | 1.00 | 26.32 | O |
| ATOM | 1033 | CB | VAL | A | 300 | 11.703 | 29.972 | 30.623 | 1.00 | 27.74 | C |
| ATOM | 1034 | CG1 | VAL | A | 300 | 10.749 | 29.958 | 31.811 | 1.00 | 29.57 | C |
| ATOM | 1035 | CG2 | VAL | A | 300 | 12.757 | 28.871 | 30.772 | 1.00 | 27.69 | C |
| ATOM | 1036 | N | THR | A | 301 | 10.420 | 31.980 | 28.432 | 1.00 | 26.55 | N |
| ATOM | 1037 | CA | THR | A | 301 | 9.539 | 33.106 | 28.142 | 1.00 | 27.35 | C |
| ATOM | 1038 | C | THR | A | 301 | 8.507 | 32.672 | 27.100 | 1.00 | 27.20 | C |
| ATOM | 1039 | O | THR | A | 301 | 7.394 | 33.188 | 27.069 | 1.00 | 27.90 | O |
| ATOM | 1040 | CB | THR | A | 301 | 10.324 | 34.329 | 27.617 | 1.00 | 27.90 | C |
| ATOM | 1041 | OG1 | THR | A | 301 | 11.097 | 33.956 | 26.472 | 1.00 | 29.74 | O |
| ATOM | 1042 | CG2 | THR | A | 301 | 11.250 | 34.861 | 28.696 | 1.00 | 29.44 | C |
| ATOM | 1043 | N | LYS | A | 302 | 8.875 | 31.715 | 26.250 | 1.00 | 26.49 | N |
| ATOM | 1044 | CA | LYS | A | 302 | 7.948 | 31.225 | 25.232 | 1.00 | 27.28 | C |
| ATOM | 1045 | C | LYS | A | 302 | 6.886 | 30.318 | 25.847 | 1.00 | 27.81 | C |
| ATOM | 1046 | O | LYS | A | 302 | 5.960 | 29.874 | 25.160 | 1.00 | 27.95 | O |
| ATOM | 1047 | CB | LYS | A | 302 | 8.701 | 30.477 | 24.130 | 1.00 | 28.36 | C |
| ATOM | 1048 | CG | LYS | A | 302 | 9.496 | 31.386 | 23.206 | 1.00 | 29.79 | C |
| ATOM | 1049 | CD | LYS | A | 302 | 10.203 | 30.586 | 22.128 | 1.00 | 30.72 | C |
| ATOM | 1050 | CE | LYS | A | 302 | 11.019 | 31.482 | 21.209 | 1.00 | 32.93 | C |
| ATOM | 1051 | NZ | LYS | A | 302 | 12.121 | 32.161 | 21.934 | 1.00 | 33.88 | N |
| ATOM | 1052 | N | ALA | A | 303 | 7.019 | 30.048 | 27.143 | 1.00 | 26.44 | N |
| ATOM | 1053 | CA | ALA | A | 303 | 6.052 | 29.219 | 27.847 | 1.00 | 27.88 | C |
| ATOM | 1054 | C | ALA | A | 303 | 5.130 | 30.097 | 28.692 | 1.00 | 28.91 | C |
| ATOM | 1055 | O | ALA | A | 303 | 4.310 | 29.592 | 29.457 | 1.00 | 29.81 | O |
| ATOM | 1056 | CB | ALA | A | 303 | 6.771 | 28.199 | 28.726 | 1.00 | 27.38 | C |
| ATOM | 1057 | N | GLY | A | 304 | 5.279 | 31.415 | 28.564 | 1.00 | 29.66 | N |
| ATOM | 1058 | CA | GLY | A | 304 | 4.423 | 32.328 | 29.309 | 1.00 | 30.57 | C |
| ATOM | 1059 | C | GLY | A | 304 | 4.963 | 32.961 | 30.582 | 1.00 | 31.32 | C |
| ATOM | 1060 | O | GLY | A | 304 | 4.257 | 33.735 | 31.234 | 1.00 | 32.07 | O |
| ATOM | 1061 | N | HIS | A | 305 | 6.202 | 32.649 | 30.948 | 1.00 | 31.10 | N |
| ATOM | 1062 | CA | HIS | A | 305 | 6.797 | 33.216 | 32.155 | 1.00 | 30.95 | C |
| ATOM | 1063 | C | HIS | A | 305 | 7.656 | 34.439 | 31.853 | 1.00 | 31.77 | C |
| ATOM | 1064 | O | HIS | A | 305 | 8.138 | 34.610 | 30.731 | 1.00 | 31.65 | O |
| ATOM | 1065 | CB | HIS | A | 305 | 7.628 | 32.155 | 32.881 | 1.00 | 30.92 | C |
| ATOM | 1066 | CG | HIS | A | 305 | 6.799 | 31.128 | 33.585 | 1.00 | 30.70 | C |
| ATOM | 1067 | ND1 | HIS | A | 305 | 6.017 | 31.430 | 34.679 | 1.00 | 31.24 | N |
| ATOM | 1068 | CD2 | HIS | A | 305 | 6.599 | 29.812 | 33.331 | 1.00 | 31.47 | C |
| ATOM | 1069 | CE1 | HIS | A | 305 | 5.369 | 30.346 | 35.067 | 1.00 | 31.89 | C |
| ATOM | 1070 | NE2 | HIS | A | 305 | 5.704 | 29.351 | 34.265 | 1.00 | 30.48 | N |
| ATOM | 1071 | N | SER | A | 306 | 7.839 | 35.290 | 32.860 | 1.00 | 32.01 | N |
| ATOM | 1072 | CA | SER | A | 306 | 8.624 | 36.511 | 32.700 | 1.00 | 33.97 | C |
| ATOM | 1073 | C | SER | A | 306 | 9.982 | 36.449 | 33.392 | 1.00 | 34.00 | C |
| ATOM | 1074 | O | SER | A | 306 | 10.265 | 35.523 | 34.154 | 1.00 | 33.09 | O |
| ATOM | 1075 | CB | SER | A | 306 | 7.842 | 37.710 | 33.235 | 1.00 | 34.42 | C |
| ATOM | 1076 | OG | SER | A | 306 | 7.739 | 37.654 | 34.645 | 1.00 | 37.62 | O |
| ATOM | 1077 | N | LEU | A | 307 | 10.813 | 37.455 | 33.125 | 1.00 | 34.07 | N |
| ATOM | 1078 | CA | LEU | A | 307 | 12.155 | 37.537 | 33.694 | 1.00 | 34.93 | C |
| ATOM | 1079 | C | LEU | A | 307 | 12.172 | 37.666 | 35.212 | 1.00 | 33.80 | C |
| ATOM | 1080 | O | LEU | A | 307 | 13.180 | 37.364 | 35.851 | 1.00 | 33.69 | O |
| ATOM | 1081 | CB | LEU | A | 307 | 12.923 | 38.710 | 33.068 | 1.00 | 36.84 | C |
| ATOM | 1082 | CG | LEU | A | 307 | 13.434 | 38.527 | 31.634 | 1.00 | 39.29 | C |
| ATOM | 1083 | CD1 | LEU | A | 307 | 12.282 | 38.235 | 30.685 | 1.00 | 40.58 | C |
| ATOM | 1084 | CD2 | LEU | A | 307 | 14.168 | 39.784 | 31.201 | 1.00 | 40.01 | C |
| ATOM | 1085 | N | GLU | A | 308 | 11.060 | 38.110 | 35.789 | 1.00 | 33.38 | N |
| ATOM | 1086 | CA | GLU | A | 308 | 10.963 | 38.265 | 37.235 | 1.00 | 32.81 | C |
| ATOM | 1087 | C | GLU | A | 308 | 11.165 | 36.913 | 37.917 | 1.00 | 31.88 | C |
| ATOM | 1088 | O | GLU | A | 308 | 11.558 | 36.842 | 39.078 | 1.00 | 30.22 | O |
| ATOM | 1089 | CB | GLU | A | 308 | 9.603 | 38.856 | 37.607 | 1.00 | 37.03 | C |
| ATOM | 1090 | CG | GLU | A | 308 | 9.308 | 40.169 | 36.888 | 1.00 | 42.70 | C |
| ATOM | 1091 | CD | GLU | A | 308 | 7.914 | 40.707 | 37.166 | 1.00 | 45.49 | C |
| ATOM | 1092 | OE1 | GLU | A | 308 | 7.522 | 41.696 | 36.507 | 1.00 | 46.94 | O |
| ATOM | 1093 | OE2 | GLU | A | 308 | 7.214 | 40.149 | 38.040 | 1.00 | 47.58 | O |
| ATOM | 1094 | N | LEU | A | 309 | 10.898 | 35.838 | 37.182 | 1.00 | 29.69 | N |
| ATOM | 1095 | CA | LEU | A | 309 | 11.081 | 34.492 | 37.714 | 1.00 | 29.34 | C |
| ATOM | 1096 | C | LEU | A | 309 | 12.348 | 33.872 | 37.130 | 1.00 | 28.31 | C |
| ATOM | 1097 | O | LEU | A | 309 | 13.160 | 33.290 | 37.848 | 1.00 | 26.92 | O |
| ATOM | 1098 | CB | LEU | A | 309 | 9.882 | 33.605 | 37.360 | 1.00 | 28.48 | C |
| ATOM | 1099 | CG | LEU | A | 309 | 10.037 | 32.116 | 37.700 | 1.00 | 28.85 | C |
| ATOM | 1100 | CD1 | LEU | A | 309 | 10.011 | 31.931 | 39.211 | 1.00 | 29.55 | C |
| ATOM | 1101 | CD2 | LEU | A | 309 | 8.919 | 31.312 | 37.048 | 1.00 | 29.07 | C |
| ATOM | 1102 | N | ILE | A | 310 | 12.524 | 34.019 | 35.822 | 1.00 | 28.87 | N |
| ATOM | 1103 | CA | ILE | A | 310 | 13.673 | 33.428 | 35.142 | 1.00 | 30.36 | C |
| ATOM | 1104 | C | ILE | A | 310 | 15.051 | 33.907 | 35.590 | 1.00 | 30.97 | C |
| ATOM | 1105 | O | ILE | A | 310 | 15.948 | 33.092 | 35.808 | 1.00 | 30.03 | O |
| ATOM | 1106 | CB | ILE | A | 310 | 13.552 | 33.605 | 33.617 | 1.00 | 31.31 | C |
| ATOM | 1107 | CG1 | ILE | A | 310 | 12.218 | 33.023 | 33.139 | 1.00 | 32.43 | C |
| ATOM | 1108 | CG2 | ILE | A | 310 | 14.695 | 32.884 | 32.918 | 1.00 | 32.83 | C |
| ATOM | 1109 | CD1 | ILE | A | 310 | 11.920 | 33.289 | 31.681 | 1.00 | 33.95 | C |
| ATOM | 1110 | N | GLU | A | 311 | 15.240 | 35.213 | 35.726 | 1.00 | 31.83 | N |
| ATOM | 1111 | CA | GLU | A | 311 | 16.547 | 35.707 | 36.151 | 1.00 | 33.28 | C |
| ATOM | 1112 | C | GLU | A | 311 | 16.945 | 35.175 | 37.528 | 1.00 | 31.76 | C |
| ATOM | 1113 | O | GLU | A | 311 | 18.067 | 34.707 | 37.714 | 1.00 | 31.24 | O |
| ATOM | 1114 | CB | GLU | A | 311 | 16.573 | 37.237 | 36.128 | 1.00 | 35.65 | C |
| ATOM | 1115 | CG | GLU | A | 311 | 16.550 | 37.788 | 34.710 | 1.00 | 41.13 | C |
| ATOM | 1116 | CD | GLU | A | 311 | 16.753 | 39.287 | 34.649 | 1.00 | 43.32 | C |
| ATOM | 1117 | OE1 | GLU | A | 311 | 16.858 | 39.815 | 33.522 | 1.00 | 46.68 | O |
| ATOM | 1118 | OE2 | GLU | A | 311 | 16.807 | 39.933 | 35.718 | 1.00 | 45.68 | O |
| ATOM | 1119 | N | PRO | A | 312 | 16.032 | 35.232 | 38.511 | 1.00 | 30.94 | N |
| ATOM | 1120 | CA | PRO | A | 312 | 16.358 | 34.728 | 39.851 | 1.00 | 29.89 | C |
| ATOM | 1121 | C | PRO | A | 312 | 16.570 | 33.212 | 39.817 | 1.00 | 28.28 | C |
| ATOM | 1122 | O | PRO | A | 312 | 17.321 | 32.656 | 40.619 | 1.00 | 28.14 | O |
| ATOM | 1123 | CB | PRO | A | 312 | 15.132 | 35.115 | 40.675 | 1.00 | 30.62 | C |
| ATOM | 1124 | CG | PRO | A | 312 | 14.612 | 36.330 | 39.962 | 1.00 | 31.93 | C |
| ATOM | 1125 | CD | PRO | A | 312 | 14.740 | 35.943 | 38.523 | 1.00 | 31.29 | C |
| ATOM | 1126 | N | LEU | A | 313 | 15.896 | 32.550 | 38.883 | 1.00 | 26.85 | N |
| ATOM | 1127 | CA | LEU | A | 313 | 16.013 | 31.102 | 38.739 | 1.00 | 26.51 | C |
| ATOM | 1128 | C | LEU | A | 313 | 17.425 | 30.764 | 38.267 | 1.00 | 25.16 | C |
| ATOM | 1129 | O | LEU | A | 313 | 18.063 | 29.855 | 38.788 | 1.00 | 24.33 | O |
| ATOM | 1130 | CB | LEU | A | 313 | 14.998 | 30.583 | 37.715 | 1.00 | 27.97 | C |
| ATOM | 1131 | CG | LEU | A | 313 | 14.373 | 29.198 | 37.935 | 1.00 | 31.36 | C |
| ATOM | 1132 | CD1 | LEU | A | 313 | 13.860 | 28.676 | 36.600 | 1.00 | 29.96 | C |
| ATOM | 1133 | CD2 | LEU | A | 313 | 15.366 | 28.230 | 38.536 | 1.00 | 30.03 | C |
| ATOM | 1134 | N | ILE | A | 314 | 17.917 | 31.504 | 37.279 | 1.00 | 25.12 | N |
| ATOM | 1135 | CA | ILE | A | 314 | 19.262 | 31.255 | 36.763 | 1.00 | 25.36 | C |
| ATOM | 1136 | C | ILE | A | 314 | 20.304 | 31.552 | 37.839 | 1.00 | 25.44 | C |
| ATOM | 1137 | O | ILE | A | 314 | 21.267 | 30.802 | 38.008 | 1.00 | 25.07 | O |
| ATOM | 1138 | CB | ILE | A | 314 | 19.565 | 32.122 | 35.517 | 1.00 | 26.51 | C |
| ATOM | 1139 | CG1 | ILE | A | 314 | 18.560 | 31.811 | 34.400 | 1.00 | 28.21 | C |
| ATOM | 1140 | CG2 | ILE | A | 314 | 20.982 | 31.844 | 35.028 | 1.00 | 26.75 | C |
| ATOM | 1141 | CD1 | ILE | A | 314 | 18.654 | 30.407 | 33.858 | 1.00 | 29.90 | C |
| ATOM | 1142 | N | LYS | A | 315 | 20.112 | 32.641 | 38.574 | 1.00 | 25.44 | N |
| ATOM | 1143 | CA | LYS | A | 315 | 21.058 | 32.994 | 39.626 | 1.00 | 26.52 | C |
| ATOM | 1144 | C | LYS | A | 315 | 21.117 | 31.869 | 40.656 | 1.00 | 25.66 | C |
| ATOM | 1145 | O | LYS | A | 315 | 22.193 | 31.522 | 41.149 | 1.00 | 25.67 | O |
| ATOM | 1146 | CB | LYS | A | 315 | 20.651 | 34.310 | 40.296 | 1.00 | 28.77 | C |
| ATOM | 1147 | CG | LYS | A | 315 | 21.759 | 34.926 | 41.134 | 1.00 | 34.86 | C |
| ATOM | 1148 | CD | LYS | A | 315 | 21.562 | 36.427 | 41.306 | 1.00 | 37.34 | C |
| ATOM | 1149 | CE | LYS | A | 315 | 22.806 | 37.082 | 41.891 | 1.00 | 39.12 | C |
| ATOM | 1150 | NZ | LYS | A | 315 | 23.154 | 36.521 | 43.227 | 1.00 | 41.56 | N |
| ATOM | 1151 | N | PHE | A | 316 | 19.958 | 31.295 | 40.967 | 1.00 | 23.92 | N |
| ATOM | 1152 | CA | PHE | A | 316 | 19.874 | 30.196 | 41.921 | 1.00 | 23.22 | C |
| ATOM | 1153 | C | PHE | A | 316 | 20.662 | 28.997 | 41.400 | 1.00 | 22.36 | C |
| ATOM | 1154 | O | PHE | A | 316 | 21.422 | 28.380 | 42.151 | 1.00 | 22.35 | O |
| ATOM | 1155 | CB | PHE | A | 316 | 18.410 | 29.791 | 42.144 | 1.00 | 24.22 | C |
| ATOM | 1156 | CG | PHE | A | 316 | 18.242 | 28.546 | 42.979 | 1.00 | 26.30 | C |
| ATOM | 1157 | CD1 | PHE | A | 316 | 18.323 | 28.605 | 44.370 | 1.00 | 27.43 | C |
| ATOM | 1158 | CD2 | PHE | A | 316 | 18.037 | 27.310 | 42.372 | 1.00 | 26.87 | C |
| ATOM | 1159 | CE1 | PHE | A | 316 | 18.204 | 27.446 | 45.141 | 1.00 | 28.46 | C |
| ATOM | 1160 | CE2 | PHE | A | 316 | 17.918 | 26.145 | 43.135 | 1.00 | 27.51 | C |
| ATOM | 1161 | CZ | PHE | A | 316 | 18.002 | 26.218 | 44.520 | 1.00 | 28.27 | C |
| ATOM | 1162 | N | GLN | A | 317 | 20.480 | 28.665 | 40.120 | 1.00 | 21.28 | N |
| ATOM | 1163 | CA | GLN | A | 317 | 21.175 | 27.524 | 39.522 | 1.00 | 21.35 | C |
| ATOM | 1164 | C | GLN | A | 317 | 22.694 | 27.681 | 39.586 | 1.00 | 21.92 | C |
| ATOM | 1165 | O | GLN | A | 317 | 23.410 | 26.735 | 39.913 | 1.00 | 20.68 | O |
| ATOM | 1166 | CB | GLN | A | 317 | 20.754 | 27.324 | 38.057 | 1.00 | 21.98 | C |
| ATOM | 1167 | CG | GLN | A | 317 | 19.296 | 26.891 | 37.855 | 1.00 | 22.78 | C |
| ATOM | 1168 | CD | GLN | A | 317 | 18.968 | 25.585 | 38.563 | 1.00 | 25.08 | C |
| ATOM | 1169 | OE1 | GLN | A | 317 | 19.792 | 24.670 | 38.619 | 1.00 | 26.08 | O |
| ATOM | 1170 | NE2 | GLN | A | 317 | 17.756 | 25.488 | 39.093 | 1.00 | 22.14 | N |
| ATOM | 1171 | N | VAL | A | 318 | 23.188 | 28.870 | 39.259 | 1.00 | 22.58 | N |
| ATOM | 1172 | CA | VAL | A | 318 | 24.629 | 29.108 | 39.301 | 1.00 | 23.76 | C |
| ATOM | 1173 | C | VAL | A | 318 | 25.162 | 28.983 | 40.734 | 1.00 | 24.71 | C |
| ATOM | 1174 | O | VAL | A | 318 | 26.199 | 28.349 | 40.971 | 1.00 | 26.38 | O |
| ATOM | 1175 | CB | VAL | A | 318 | 24.975 | 30.510 | 38.727 | 1.00 | 24.56 | C |
| ATOM | 1176 | CG1 | VAL | A | 318 | 26.458 | 30.798 | 38.897 | 1.00 | 26.05 | C |
| ATOM | 1177 | CG2 | VAL | A | 318 | 24.608 | 30.567 | 37.255 | 1.00 | 23.60 | C |
| ATOM | 1178 | N | GLY | A | 319 | 24.447 | 29.574 | 41.687 | 1.00 | 25.34 | N |
| ATOM | 1179 | CA | GLY | A | 319 | 24.868 | 29.515 | 43.076 | 1.00 | 26.42 | C |
| ATOM | 1180 | C | GLY | A | 319 | 24.892 | 28.099 | 43.623 | 1.00 | 26.70 | C |
| ATOM | 1181 | O | GLY | A | 319 | 25.778 | 27.738 | 44.399 | 1.00 | 26.15 | O |
| ATOM | 1182 | N | LEU | A | 320 | 23.915 | 27.292 | 43.226 | 1.00 | 25.08 | N |
| ATOM | 1183 | CA | LEU | A | 320 | 23.856 | 25.910 | 43.680 | 1.00 | 26.49 | C |
| ATOM | 1184 | C | LEU | A | 320 | 25.001 | 25.141 | 43.019 | 1.00 | 26.16 | C |
| ATOM | 1185 | O | LEU | A | 320 | 25.674 | 24.342 | 43.666 | 1.00 | 25.62 | O |
| ATOM | 1186 | CB | LEU | A | 320 | 22.499 | 25.289 | 43.318 | 1.00 | 26.17 | C |
| ATOM | 1187 | CG | LEU | A | 320 | 22.202 | 23.895 | 43.877 | 1.00 | 29.00 | C |
| ATOM | 1188 | CD1 | LEU | A | 320 | 22.305 | 23.911 | 45.394 | 1.00 | 28.44 | C |
| ATOM | 1189 | CD2 | LEU | A | 320 | 20.803 | 23.457 | 43.439 | 1.00 | 27.28 | C |
| ATOM | 1190 | N | LYS | A | 321 | 25.231 | 25.402 | 41.734 | 1.00 | 26.97 | N |
| ATOM | 1191 | CA | LYS | A | 321 | 26.312 | 24.743 | 41.000 | 1.00 | 29.33 | C |
| ATOM | 1192 | C | LYS | A | 321 | 27.664 | 24.983 | 41.649 | 1.00 | 30.36 | C |
| ATOM | 1193 | O | LYS | A | 321 | 28.486 | 24.070 | 41.746 | 1.00 | 30.00 | O |
| ATOM | 1194 | CB | LYS | A | 321 | 26.385 | 25.252 | 39.561 | 1.00 | 30.33 | C |
| ATOM | 1195 | CG | LYS | A | 321 | 25.578 | 24.465 | 38.559 | 1.00 | 33.36 | C |
| ATOM | 1196 | CD | LYS | A | 321 | 26.140 | 23.069 | 38.341 | 1.00 | 33.34 | C |
| ATOM | 1197 | CE | LYS | A | 321 | 25.279 | 22.329 | 37.336 | 1.00 | 33.36 | C |
| ATOM | 1198 | NZ | LYS | A | 321 | 25.668 | 20.911 | 37.111 | 1.00 | 32.77 | N |
| ATOM | 1199 | N | LYS | A | 322 | 27.894 | 26.222 | 42.077 | 1.00 | 30.82 | N |
| ATOM | 1200 | CA | LYS | A | 322 | 29.155 | 26.601 | 42.702 | 1.00 | 32.19 | C |
| ATOM | 1201 | C | LYS | A | 322 | 29.447 | 25.934 | 44.037 | 1.00 | 32.03 | C |
| ATOM | 1202 | O | LYS | A | 322 | 30.598 | 25.896 | 44.462 | 1.00 | 32.87 | O |
| ATOM | 1203 | CB | LYS | A | 322 | 29.234 | 28.122 | 42.866 | 1.00 | 33.78 | C |
| ATOM | 1204 | CG | LYS | A | 322 | 29.592 | 28.853 | 41.587 | 1.00 | 37.24 | C |
| ATOM | 1205 | CD | LYS | A | 322 | 29.849 | 30.328 | 41.856 | 1.00 | 39.61 | C |
| ATOM | 1206 | CE | LYS | A | 322 | 30.611 | 30.964 | 40.712 | 1.00 | 41.25 | C |
| ATOM | 1207 | NZ | LYS | A | 322 | 31.956 | 30.335 | 40.544 | 1.00 | 43.80 | N |
| ATOM | 1208 | N | LEU | A | 323 | 28.420 | 25.415 | 44.703 | 1.00 | 30.51 | N |
| ATOM | 1209 | CA | LEU | A | 323 | 28.627 | 24.747 | 45.985 | 1.00 | 31.09 | C |
| ATOM | 1210 | C | LEU | A | 323 | 29.296 | 23.392 | 45.774 | 1.00 | 31.05 | C |
| ATOM | 1211 | O | LEU | A | 323 | 29.833 | 22.805 | 46.715 | 1.00 | 31.05 | O |
| ATOM | 1212 | CB | LEU | A | 323 | 27.297 | 24.544 | 46.719 | 1.00 | 30.29 | C |
| ATOM | 1213 | CG | LEU | A | 323 | 26.551 | 25.784 | 47.220 | 1.00 | 31.62 | C |
| ATOM | 1214 | CD1 | LEU | A | 323 | 25.260 | 25.359 | 47.904 | 1.00 | 30.41 | C |
| ATOM | 1215 | CD2 | LEU | A | 323 | 27.434 | 26.570 | 48.180 | 1.00 | 31.32 | C |
| ATOM | 1216 | N | ASN | A | 324 | 29.264 | 22.908 | 44.535 | 1.00 | 30.91 | N |
| ATOM | 1217 | CA | ASN | A | 324 | 29.854 | 21.619 | 44.180 | 1.00 | 32.42 | C |
| ATOM | 1218 | C | ASN | A | 324 | 29.466 | 20.524 | 45.165 | 1.00 | 32.07 | C |
| ATOM | 1219 | O | ASN | A | 324 | 30.323 | 19.864 | 45.755 | 1.00 | 32.62 | O |
| ATOM | 1220 | CB | ASN | A | 324 | 31.380 | 21.722 | 44.110 | 1.00 | 36.14 | C |
| ATOM | 1221 | CG | ASN | A | 324 | 31.853 | 22.576 | 42.954 | 1.00 | 38.53 | C |
| ATOM | 1222 | OD1 | ASN | A | 324 | 32.013 | 23.789 | 43.087 | 1.00 | 43.04 | O |
| ATOM | 1223 | ND2 | ASN | A | 324 | 32.068 | 21.947 | 41.805 | 1.00 | 40.87 | N |
| ATOM | 1224 | N | LEU | A | 325 | 28.166 | 20.326 | 45.333 | 1.00 | 29.80 | N |
| ATOM | 1225 | CA | LEU | A | 325 | 27.667 | 19.320 | 46.257 | 1.00 | 27.98 | C |
| ATOM | 1226 | C | LEU | A | 325 | 27.969 | 17.890 | 45.836 | 1.00 | 27.42 | C |
| ATOM | 1227 | O | LEU | A | 325 | 27.984 | 17.568 | 44.648 | 1.00 | 27.50 | O |
| ATOM | 1228 | CB | LEU | A | 325 | 26.149 | 19.454 | 46.409 | 1.00 | 28.15 | C |
| ATOM | 1229 | CG | LEU | A | 325 | 25.592 | 20.785 | 46.907 | 1.00 | 28.88 | C |
| ATOM | 1230 | CD1 | LEU | A | 325 | 24.072 | 20.701 | 46.960 | 1.00 | 29.23 | C |
| ATOM | 1231 | CD2 | LEU | A | 325 | 26.163 | 21.105 | 48.276 | 1.00 | 28.09 | C |
| ATOM | 1232 | N | HIS | A | 326 | 28.219 | 17.033 | 46.821 | 1.00 | 26.59 | N |
| ATOM | 1233 | CA | HIS | A | 326 | 28.430 | 15.618 | 46.546 | 1.00 | 25.79 | C |
| ATOM | 1234 | C | HIS | A | 326 | 27.003 | 15.162 | 46.264 | 1.00 | 25.33 | C |
| ATOM | 1235 | O | HIS | A | 326 | 26.052 | 15.819 | 46.695 | 1.00 | 23.44 | O |
| ATOM | 1236 | CB | HIS | A | 326 | 28.935 | 14.882 | 47.788 | 1.00 | 27.17 | C |
| ATOM | 1237 | CG | HIS | A | 326 | 30.303 | 15.294 | 48.231 | 1.00 | 27.36 | C |
| ATOM | 1238 | ND1 | HIS | A | 326 | 30.942 | 14.704 | 49.301 | 1.00 | 28.09 | N |
| ATOM | 1239 | CD2 | HIS | A | 326 | 31.159 | 16.222 | 47.744 | 1.00 | 28.85 | C |
| ATOM | 1240 | CE1 | HIS | A | 326 | 32.135 | 15.251 | 49.453 | 1.00 | 28.02 | C |
| ATOM | 1241 | NE2 | HIS | A | 326 | 32.292 | 16.174 | 48.521 | 1.00 | 29.20 | N |
| ATOM | 1242 | N | GLU | A | 327 | 26.839 | 14.054 | 45.554 | 1.00 | 24.49 | N |
| ATOM | 1243 | CA | GLU | A | 327 | 25.497 | 13.569 | 45.267 | 1.00 | 24.94 | C |
| ATOM | 1244 | C | GLU | A | 327 | 24.768 | 13.297 | 46.583 | 1.00 | 24.29 | C |
| ATOM | 1245 | O | GLU | A | 327 | 23.553 | 13.498 | 46.686 | 1.00 | 24.42 | O |
| ATOM | 1246 | CB | GLU | A | 327 | 25.557 | 12.302 | 44.409 | 1.00 | 27.30 | C |
| ATOM | 1247 | CG | GLU | A | 327 | 24.185 | 11.755 | 44.032 | 1.00 | 29.69 | C |
| ATOM | 1248 | CD | GLU | A | 327 | 24.247 | 10.740 | 42.903 | 1.00 | 32.63 | C |
| ATOM | 1249 | OE1 | GLU | A | 327 | 25.021 | 9.771 | 43.015 | 1.00 | 31.56 | O |
| ATOM | 1250 | OE2 | GLU | A | 327 | 23.519 | 10.915 | 41.903 | 1.00 | 32.79 | O |
| ATOM | 1251 | N | GLU | A | 328 | 25.516 | 12.858 | 47.595 | 1.00 | 22.79 | N |
| ATOM | 1252 | CA | GLU | A | 328 | 24.942 | 12.576 | 48.911 | 1.00 | 23.11 | C |
| ATOM | 1253 | C | GLU | A | 328 | 24.280 | 13.822 | 49.500 | 1.00 | 23.46 | C |
| ATOM | 1254 | O | GLU | A | 328 | 23.199 | 13.750 | 50.086 | 1.00 | 23.51 | O |
| ATOM | 1255 | CB | GLU | A | 328 | 26.025 | 12.083 | 49.877 | 1.00 | 24.71 | C |
| ATOM | 1256 | CG | GLU | A | 328 | 26.540 | 10.666 | 49.607 | 1.00 | 25.97 | C |
| ATOM | 1257 | CD | GLU | A | 328 | 27.584 | 10.591 | 48.506 | 1.00 | 28.78 | C |
| ATOM | 1258 | OE1 | GLU | A | 328 | 28.201 | 9.512 | 48.356 | 1.00 | 29.81 | O |
| ATOM | 1259 | OE2 | GLU | A | 328 | 27.793 | 11.591 | 47.789 | 1.00 | 27.61 | O |
| ATOM | 1260 | N | GLU | A | 329 | 24.939 | 14.965 | 49.349 | 1.00 | 22.58 | N |
| ATOM | 1261 | CA | GLU | A | 329 | 24.406 | 16.221 | 49.861 | 1.00 | 23.12 | C |
| ATOM | 1262 | C | GLU | A | 329 | 23.212 | 16.678 | 49.026 | 1.00 | 22.73 | C |
| ATOM | 1263 | O | GLU | A | 329 | 22.236 | 17.203 | 49.558 | 1.00 | 22.26 | O |
| ATOM | 1264 | CB | GLU | A | 329 | 25.511 | 17.281 | 49.856 | 1.00 | 23.27 | C |
| ATOM | 1265 | CG | GLU | A | 329 | 26.608 | 16.943 | 50.859 | 1.00 | 24.71 | C |
| ATOM | 1266 | CD | GLU | A | 329 | 27.940 | 17.599 | 50.554 | 1.00 | 26.09 | C |
| ATOM | 1267 | OE1 | GLU | A | 329 | 28.825 | 17.532 | 51.429 | 1.00 | 27.57 | O |
| ATOM | 1268 | OE2 | GLU | A | 329 | 28.113 | 18.160 | 49.454 | 1.00 | 26.68 | O |
| ATOM | 1269 | N | HIS | A | 330 | 23.291 | 16.450 | 47.721 | 1.00 | 22.72 | N |
| ATOM | 1270 | CA | HIS | A | 330 | 22.225 | 16.836 | 46.803 | 1.00 | 22.97 | C |
| ATOM | 1271 | C | HIS | A | 330 | 20.908 | 16.139 | 47.150 | 1.00 | 23.43 | C |
| ATOM | 1272 | O | HIS | A | 330 | 19.863 | 16.790 | 47.257 | 1.00 | 22.10 | O |
| ATOM | 1273 | CB | HIS | A | 330 | 22.638 | 16.494 | 45.364 | 1.00 | 24.13 | C |
| ATOM | 1274 | CG | HIS | A | 330 | 21.648 | 16.916 | 44.321 | 1.00 | 25.22 | C |
| ATOM | 1275 | ND1 | HIS | A | 330 | 21.357 | 18.237 | 44.060 | 1.00 | 25.99 | N |
| ATOM | 1276 | CD2 | HIS | A | 330 | 20.913 | 16.190 | 43.444 | 1.00 | 25.76 | C |
| ATOM | 1277 | CE1 | HIS | A | 330 | 20.489 | 18.307 | 43.065 | 1.00 | 26.73 | C |
| ATOM | 1278 | NE2 | HIS | A | 330 | 20.203 | 17.078 | 42.674 | 1.00 | 25.08 | N |
| ATOM | 1279 | N | VAL | A | 331 | 20.955 | 14.823 | 47.334 | 1.00 | 22.22 | N |
| ATOM | 1280 | CA | VAL | A | 331 | 19.739 | 14.072 | 47.642 | 1.00 | 23.00 | C |
| ATOM | 1281 | C | VAL | A | 331 | 19.185 | 14.382 | 49.024 | 1.00 | 22.12 | C |
| ATOM | 1282 | O | VAL | A | 331 | 17.968 | 14.393 | 49.218 | 1.00 | 21.17 | O |
| ATOM | 1283 | CB | VAL | A | 331 | 19.952 | 12.544 | 47.490 | 1.00 | 22.74 | C |
| ATOM | 1284 | CG1 | VAL | A | 331 | 20.363 | 12.233 | 46.053 | 1.00 | 25.60 | C |
| ATOM | 1285 | CG2 | VAL | A | 331 | 21.008 | 12.045 | 48.466 | 1.00 | 25.97 | C |
| ATOM | 1286 | N | LEU | A | 332 | 20.067 | 14.634 | 49.986 | 1.00 | 21.61 | N |
| ATOM | 1287 | CA | LEU | A | 332 | 19.611 | 14.967 | 51.327 | 1.00 | 21.81 | C |
| ATOM | 1288 | C | LEU | A | 332 | 18.884 | 16.311 | 51.301 | 1.00 | 21.82 | C |
| ATOM | 1289 | O | LEU | A | 332 | 17.874 | 16.489 | 51.976 | 1.00 | 22.23 | O |
| ATOM | 1290 | CB | LEU | A | 332 | 20.796 | 15.020 | 52.303 | 1.00 | 22.40 | C |
| ATOM | 1291 | CG | LEU | A | 332 | 21.262 | 13.656 | 52.824 | 1.00 | 22.71 | C |
| ATOM | 1292 | CD1 | LEU | A | 332 | 22.617 | 13.777 | 53.516 | 1.00 | 23.21 | C |
| ATOM | 1293 | CD2 | LEU | A | 332 | 20.214 | 13.112 | 53.776 | 1.00 | 23.85 | C |
| ATOM | 1294 | N | LEU | A | 333 | 19.389 | 17.253 | 50.508 | 1.00 | 21.45 | N |
| ATOM | 1295 | CA | LEU | A | 333 | 18.763 | 18.569 | 50.420 | 1.00 | 22.43 | C |
| ATOM | 1296 | C | LEU | A | 333 | 17.363 | 18.478 | 49.808 | 1.00 | 21.61 | C |
| ATOM | 1297 | O | LEU | A | 333 | 16.440 | 19.157 | 50.259 | 1.00 | 21.39 | O |
| ATOM | 1298 | CB | LEU | A | 333 | 19.637 | 19.521 | 49.599 | 1.00 | 23.63 | C |
| ATOM | 1299 | CG | LEU | A | 333 | 19.221 | 21.000 | 49.597 | 1.00 | 26.05 | C |
| ATOM | 1300 | CD1 | LEU | A | 333 | 19.253 | 21.557 | 51.014 | 1.00 | 26.27 | C |
| ATOM | 1301 | CD2 | LEU | A | 333 | 20.157 | 21.785 | 48.703 | 1.00 | 26.03 | C |
| ATOM | 1302 | N | MET | A | 334 | 17.198 | 17.654 | 48.776 | 1.00 | 21.27 | N |
| ATOM | 1303 | CA | MET | A | 334 | 15.878 | 17.513 | 48.163 | 1.00 | 20.93 | C |
| ATOM | 1304 | C | MET | A | 334 | 14.928 | 16.881 | 49.171 | 1.00 | 21.48 | C |
| ATOM | 1305 | O | MET | A | 334 | 13.769 | 17.263 | 49.256 | 1.00 | 21.52 | O |
| ATOM | 1306 | CB | MET | A | 334 | 15.939 | 16.648 | 46.896 | 1.00 | 21.53 | C |
| ATOM | 1307 | CG | MET | A | 334 | 16.631 | 17.318 | 45.719 | 1.00 | 22.31 | C |
| ATOM | 1308 | SD | MET | A | 334 | 16.442 | 16.343 | 44.219 | 1.00 | 24.84 | S |
| ATOM | 1309 | CE | MET | A | 334 | 17.484 | 14.909 | 44.612 | 1.00 | 24.19 | C |
| ATOM | 1310 | N | ALA | A | 335 | 15.427 | 15.922 | 49.950 | 1.00 | 21.64 | N |
| ATOM | 1311 | CA | ALA | A | 335 | 14.596 | 15.255 | 50.949 | 1.00 | 21.82 | C |
| ATOM | 1312 | C | ALA | A | 335 | 14.167 | 16.231 | 52.045 | 1.00 | 22.81 | C |
| ATOM | 1313 | O | ALA | A | 335 | 13.002 | 16.248 | 52.455 | 1.00 | 22.95 | O |
| ATOM | 1314 | CB | ALA | A | 335 | 15.355 | 14.070 | 51.564 | 1.00 | 22.44 | C |
| ATOM | 1315 | N | ILE | A | 336 | 15.111 | 17.041 | 52.517 | 1.00 | 21.90 | N |
| ATOM | 1316 | CA | ILE | A | 336 | 14.827 | 18.022 | 53.560 | 1.00 | 22.98 | C |
| ATOM | 1317 | C | ILE | A | 336 | 13.822 | 19.050 | 53.038 | 1.00 | 23.92 | C |
| ATOM | 1318 | O | ILE | A | 336 | 12.949 | 19.496 | 53.772 | 1.00 | 23.55 | O |
| ATOM | 1319 | CB | ILE | A | 336 | 16.129 | 18.730 | 54.020 | 1.00 | 23.77 | C |
| ATOM | 1320 | CG1 | ILE | A | 336 | 17.021 | 17.724 | 54.753 | 1.00 | 24.24 | C |
| ATOM | 1321 | CG2 | ILE | A | 336 | 15.803 | 19.914 | 54.936 | 1.00 | 25.02 | C |
| ATOM | 1322 | CD1 | ILE | A | 336 | 18.445 | 18.188 | 54.950 | 1.00 | 27.51 | C |
| ATOM | 1323 | N | CYS | A | 337 | 13.942 | 19.411 | 51.765 | 1.00 | 22.82 | N |
| ATOM | 1324 | CA | CYS | A | 337 | 13.020 | 20.365 | 51.166 | 1.00 | 23.92 | C |
| ATOM | 1325 | C | CYS | A | 337 | 11.582 | 19.846 | 51.235 | 1.00 | 24.00 | C |
| ATOM | 1326 | O | CYS | A | 337 | 10.665 | 20.577 | 51.605 | 1.00 | 25.45 | O |
| ATOM | 1327 | CB | CYS | A | 337 | 13.410 | 20.622 | 49.705 | 1.00 | 22.95 | C |
| ATOM | 1328 | SG | CYS | A | 337 | 12.289 | 21.736 | 48.817 | 1.00 | 25.85 | S |
| ATOM | 1329 | N | ILE | A | 338 | 11.393 | 18.578 | 50.886 | 1.00 | 23.65 | N |
| ATOM | 1330 | CA | ILE | A | 338 | 10.070 | 17.957 | 50.890 | 1.00 | 23.56 | C |
| ATOM | 1331 | C | ILE | A | 338 | 9.457 | 17.814 | 52.284 | 1.00 | 26.02 | C |
| ATOM | 1332 | O | ILE | A | 338 | 8.288 | 18.153 | 52.501 | 1.00 | 25.70 | O |
| ATOM | 1333 | CB | ILE | A | 338 | 10.126 | 16.560 | 50.231 | 1.00 | 23.28 | C |
| ATOM | 1334 | CG1 | ILE | A | 338 | 10.483 | 16.704 | 48.746 | 1.00 | 22.98 | C |
| ATOM | 1335 | CG2 | ILE | A | 338 | 8.794 | 15.839 | 50.396 | 1.00 | 24.00 | C |
| ATOM | 1336 | CD1 | ILE | A | 338 | 10.807 | 15.387 | 48.057 | 1.00 | 22.98 | C |
| ATOM | 1337 | N | VAL | A | 339 | 10.242 | 17.305 | 53.225 | 1.00 | 26.29 | N |
| ATOM | 1338 | CA | VAL | A | 339 | 9.754 | 17.106 | 54.584 | 1.00 | 29.21 | C |
| ATOM | 1339 | C | VAL | A | 339 | 9.971 | 18.359 | 55.430 | 1.00 | 29.45 | C |
| ATOM | 1340 | O | VAL | A | 339 | 10.807 | 18.378 | 56.333 | 1.00 | 30.57 | O |
| ATOM | 1341 | CB | VAL | A | 339 | 10.461 | 15.901 | 55.241 | 1.00 | 30.37 | C |
| ATOM | 1342 | CG1 | VAL | A | 339 | 9.751 | 15.516 | 56.524 | 1.00 | 31.20 | C |
| ATOM | 1343 | CG2 | VAL | A | 339 | 10.479 | 14.725 | 54.277 | 1.00 | 31.68 | C |
| ATOM | 1344 | N | SER | A | 340 | 9.213 | 19.407 | 55.122 | 1.00 | 30.04 | N |
| ATOM | 1345 | CA | SER | A | 340 | 9.309 | 20.676 | 55.842 | 1.00 | 30.94 | C |
| ATOM | 1346 | C | SER | A | 340 | 8.061 | 20.868 | 56.701 | 1.00 | 31.69 | C |
| ATOM | 1347 | O | SER | A | 340 | 6.940 | 20.841 | 56.195 | 1.00 | 31.64 | O |
| ATOM | 1348 | CB | SER | A | 340 | 9.438 | 21.838 | 54.853 | 1.00 | 32.39 | C |
| ATOM | 1349 | OG | SER | A | 340 | 10.664 | 21.773 | 54.142 | 1.00 | 35.78 | O |
| ATOM | 1350 | N | PRO | A | 341 | 8.243 | 21.075 | 58.013 | 1.00 | 32.38 | N |
| ATOM | 1351 | CA | PRO | A | 341 | 7.107 | 21.263 | 58.919 | 1.00 | 33.82 | C |
| ATOM | 1352 | C | PRO | A | 341 | 6.344 | 22.579 | 58.774 | 1.00 | 35.40 | C |
| ATOM | 1353 | O | PRO | A | 341 | 5.204 | 22.688 | 59.232 | 1.00 | 36.23 | O |
| ATOM | 1354 | CB | PRO | A | 341 | 7.745 | 21.111 | 60.298 | 1.00 | 33.68 | C |
| ATOM | 1355 | CG | PRO | A | 341 | 9.110 | 21.675 | 60.094 | 1.00 | 33.78 | C |
| ATOM | 1356 | CD | PRO | A | 341 | 9.517 | 21.088 | 58.754 | 1.00 | 32.78 | C |
| ATOM | 1357 | N | ASP | A | 342 | 6.954 | 23.570 | 58.131 | 1.00 | 36.36 | N |
| ATOM | 1358 | CA | ASP | A | 342 | 6.301 | 24.866 | 57.981 | 1.00 | 37.82 | C |
| ATOM | 1359 | C | ASP | A | 342 | 5.580 | 25.094 | 56.657 | 1.00 | 38.30 | C |
| ATOM | 1360 | O | ASP | A | 342 | 5.655 | 26.181 | 56.084 | 1.00 | 39.93 | O |
| ATOM | 1361 | CB | ASP | A | 342 | 7.304 | 26.001 | 58.213 | 1.00 | 39.62 | C |
| ATOM | 1362 | CG | ASP | A | 342 | 8.441 | 25.987 | 57.218 | 1.00 | 41.16 | C |
| ATOM | 1363 | OD1 | ASP | A | 342 | 9.185 | 26.989 | 57.152 | 1.00 | 43.27 | O |
| ATOM | 1364 | OD2 | ASP | A | 342 | 8.597 | 24.974 | 56.505 | 1.00 | 42.71 | O |
| ATOM | 1365 | N | ARG | A | 343 | 4.887 | 24.072 | 56.170 | 1.00 | 37.31 | N |
| ATOM | 1366 | CA | ARG | A | 343 | 4.123 | 24.195 | 54.933 | 1.00 | 37.10 | C |
| ATOM | 1367 | C | ARG | A | 343 | 2.683 | 24.409 | 55.375 | 1.00 | 37.98 | C |
| ATOM | 1368 | O | ARG | A | 343 | 2.198 | 23.723 | 56.273 | 1.00 | 38.15 | O |
| ATOM | 1369 | CB | ARG | A | 343 | 4.207 | 22.911 | 54.103 | 1.00 | 35.71 | C |
| ATOM | 1370 | CG | ARG | A | 343 | 5.595 | 22.543 | 53.612 | 1.00 | 32.83 | C |
| ATOM | 1371 | CD | ARG | A | 343 | 6.123 | 23.513 | 52.565 | 1.00 | 31.70 | C |
| ATOM | 1372 | NE | ARG | A | 343 | 7.282 | 22.947 | 51.879 | 1.00 | 29.77 | N |
| ATOM | 1373 | CZ | ARG | A | 343 | 8.062 | 23.612 | 51.032 | 1.00 | 28.95 | C |
| ATOM | 1374 | NH1 | ARG | A | 343 | 7.818 | 24.886 | 50.751 | 1.00 | 27.66 | N |
| ATOM | 1375 | NH2 | ARG | A | 343 | 9.097 | 23.002 | 50.472 | 1.00 | 28.12 | N |
| ATOM | 1376 | N | PRO | A | 344 | 1.979 | 25.367 | 54.761 | 1.00 | 39.05 | N |
| ATOM | 1377 | CA | PRO | A | 344 | 0.592 | 25.579 | 55.180 | 1.00 | 39.38 | C |
| ATOM | 1378 | C | PRO | A | 344 | −0.279 | 24.351 | 54.924 | 1.00 | 38.87 | C |
| ATOM | 1379 | O | PRO | A | 344 | −0.205 | 23.742 | 53.858 | 1.00 | 39.29 | O |
| ATOM | 1380 | CB | PRO | A | 344 | 0.167 | 26.786 | 54.348 | 1.00 | 39.90 | C |
| ATOM | 1381 | CG | PRO | A | 344 | 0.974 | 26.624 | 53.092 | 1.00 | 40.56 | C |
| ATOM | 1382 | CD | PRO | A | 344 | 2.332 | 26.243 | 53.630 | 1.00 | 39.66 | C |
| ATOM | 1383 | N | GLY | A | 345 | −1.085 | 23.978 | 55.912 | 1.00 | 38.50 | N |
| ATOM | 1384 | CA | GLY | A | 345 | −1.965 | 22.836 | 55.746 | 1.00 | 37.96 | C |
| ATOM | 1385 | C | GLY | A | 345 | −1.567 | 21.544 | 56.437 | 1.00 | 37.94 | C |
| ATOM | 1386 | O | GLY | A | 345 | −2.386 | 20.630 | 56.537 | 1.00 | 36.75 | O |
| ATOM | 1387 | N | VAL | A | 346 | −0.328 | 21.452 | 56.914 | 1.00 | 37.90 | N |
| ATOM | 1388 | CA | VAL | A | 346 | 0.125 | 20.234 | 57.585 | 1.00 | 38.39 | C |
| ATOM | 1389 | C | VAL | A | 346 | −0.584 | 20.046 | 58.922 | 1.00 | 39.30 | C |
| ATOM | 1390 | O | VAL | A | 346 | −0.832 | 21.012 | 59.643 | 1.00 | 39.35 | O |
| ATOM | 1391 | CB | VAL | A | 346 | 1.654 | 20.249 | 57.827 | 1.00 | 38.81 | C |
| ATOM | 1392 | CG1 | VAL | A | 346 | 2.383 | 20.409 | 56.503 | 1.00 | 37.92 | C |
| ATOM | 1393 | CG2 | VAL | A | 346 | 2.030 | 21.366 | 58.784 | 1.00 | 38.02 | C |
| ATOM | 1394 | N | GLN | A | 347 | −0.905 | 18.796 | 59.247 | 1.00 | 39.79 | N |
| ATOM | 1395 | CA | GLN | A | 347 | −1.597 | 18.481 | 60.492 | 1.00 | 40.19 | C |
| ATOM | 1396 | C | GLN | A | 347 | −0.631 | 18.135 | 61.612 | 1.00 | 38.86 | C |
| ATOM | 1397 | O | GLN | A | 347 | −0.657 | 18.758 | 62.673 | 1.00 | 39.58 | O |
| ATOM | 1398 | CB | GLN | A | 347 | −2.564 | 17.314 | 60.280 | 1.00 | 43.00 | C |
| ATOM | 1399 | CG | GLN | A | 347 | −3.565 | 17.531 | 59.157 | 1.00 | 47.50 | C |
| ATOM | 1400 | CD | GLN | A | 347 | −4.526 | 18.678 | 59.423 | 1.00 | 50.27 | C |
| ATOM | 1401 | OE1 | GLN | A | 347 | −4.582 | 19.209 | 60.535 | 1.00 | 52.52 | O |
| ATOM | 1402 | NE2 | GLN | A | 347 | −5.283 | 19.071 | 58.402 | 1.00 | 51.48 | N |
| ATOM | 1403 | N | ASP | A | 348 | 0.223 | 17.144 | 61.380 | 1.00 | 36.34 | N |
| ATOM | 1404 | CA | ASP | A | 348 | 1.181 | 16.730 | 62.398 | 1.00 | 35.34 | C |
| ATOM | 1405 | C | ASP | A | 348 | 2.568 | 17.313 | 62.152 | 1.00 | 33.83 | C |
| ATOM | 1406 | O | ASP | A | 348 | 3.474 | 16.622 | 61.679 | 1.00 | 33.85 | O |
| ATOM | 1407 | CB | ASP | A | 348 | 1.257 | 15.203 | 62.458 | 1.00 | 34.84 | C |
| ATOM | 1408 | CG | ASP | A | 348 | 1.947 | 14.707 | 63.712 | 1.00 | 35.23 | C |
| ATOM | 1409 | OD1 | ASP | A | 348 | 1.907 | 13.488 | 63.972 | 1.00 | 34.74 | O |
| ATOM | 1410 | OD2 | ASP | A | 348 | 2.531 | 15.539 | 64.437 | 1.00 | 34.97 | O |
| ATOM | 1411 | N | ALA | A | 349 | 2.727 | 18.587 | 62.492 | 1.00 | 32.41 | N |
| ATOM | 1412 | CA | ALA | A | 349 | 3.991 | 19.286 | 62.307 | 1.00 | 32.51 | C |
| ATOM | 1413 | C | ALA | A | 349 | 5.122 | 18.665 | 63.121 | 1.00 | 32.68 | C |
| ATOM | 1414 | O | ALA | A | 349 | 6.263 | 18.602 | 62.662 | 1.00 | 32.47 | O |
| ATOM | 1415 | CB | ALA | A | 349 | 3.829 | 20.753 | 62.677 | 1.00 | 32.86 | C |
| ATOM | 1416 | N | ALA | A | 350 | 4.804 | 18.206 | 64.328 | 1.00 | 31.95 | N |
| ATOM | 1417 | CA | ALA | A | 350 | 5.809 | 17.602 | 65.200 | 1.00 | 31.15 | C |
| ATOM | 1418 | C | ALA | A | 350 | 6.458 | 16.367 | 64.578 | 1.00 | 30.76 | C |
| ATOM | 1419 | O | ALA | A | 350 | 7.676 | 16.190 | 64.655 | 1.00 | 30.22 | O |
| ATOM | 1420 | CB | ALA | A | 350 | 5.180 | 17.240 | 66.547 | 1.00 | 32.37 | C |
| ATOM | 1421 | N | LEU | A | 351 | 5.643 | 15.510 | 63.972 | 1.00 | 30.64 | N |
| ATOM | 1422 | CA | LEU | A | 351 | 6.150 | 14.298 | 63.340 | 1.00 | 30.92 | C |
| ATOM | 1423 | C | LEU | A | 351 | 7.032 | 14.690 | 62.156 | 1.00 | 30.72 | C |
| ATOM | 1424 | O | LEU | A | 351 | 8.137 | 14.181 | 61.995 | 1.00 | 30.35 | O |
| ATOM | 1425 | CB | LEU | A | 351 | 4.989 | 13.428 | 62.848 | 1.00 | 32.92 | C |
| ATOM | 1426 | CG | LEU | A | 351 | 5.214 | 11.919 | 62.690 | 1.00 | 34.73 | C |
| ATOM | 1427 | CD1 | LEU | A | 351 | 4.073 | 11.326 | 61.881 | 1.00 | 35.25 | C |
| ATOM | 1428 | CD2 | LEU | A | 351 | 6.528 | 11.640 | 62.005 | 1.00 | 36.86 | C |
| ATOM | 1429 | N | ILE | A | 352 | 6.531 | 15.597 | 61.325 | 1.00 | 30.54 | N |
| ATOM | 1430 | CA | ILE | A | 352 | 7.282 | 16.056 | 60.158 | 1.00 | 29.35 | C |
| ATOM | 1431 | C | ILE | A | 352 | 8.628 | 16.646 | 60.580 | 1.00 | 29.73 | C |
| ATOM | 1432 | O | ILE | A | 352 | 9.658 | 16.371 | 59.959 | 1.00 | 30.37 | O |
| ATOM | 1433 | CB | ILE | A | 352 | 6.465 | 17.107 | 59.362 | 1.00 | 29.44 | C |
| ATOM | 1434 | CG1 | ILE | A | 352 | 5.175 | 16.463 | 58.842 | 1.00 | 29.58 | C |
| ATOM | 1435 | CG2 | ILE | A | 352 | 7.290 | 17.647 | 58.193 | 1.00 | 28.41 | C |
| ATOM | 1436 | CD1 | ILE | A | 352 | 4.166 | 17.452 | 58.284 | 1.00 | 29.04 | C |
| ATOM | 1437 | N | GLU | A | 353 | 8.626 | 17.445 | 61.644 | 1.00 | 30.02 | N |
| ATOM | 1438 | CA | GLU | A | 353 | 9.857 | 18.058 | 62.130 | 1.00 | 30.56 | C |
| ATOM | 1439 | C | GLU | A | 353 | 10.845 | 17.000 | 62.613 | 1.00 | 29.99 | C |
| ATOM | 1440 | O | GLU | A | 353 | 12.050 | 17.147 | 62.438 | 1.00 | 29.97 | O |
| ATOM | 1441 | CB | GLU | A | 353 | 9.565 | 19.048 | 63.266 | 1.00 | 32.59 | C |
| ATOM | 1442 | CG | GLU | A | 353 | 10.755 | 19.941 | 63.615 | 1.00 | 35.81 | C |
| ATOM | 1443 | CD | GLU | A | 353 | 10.462 | 20.922 | 64.740 | 1.00 | 38.69 | C |
| ATOM | 1444 | OE1 | GLU | A | 353 | 9.381 | 21.551 | 64.723 | 1.00 | 40.67 | O |
| ATOM | 1445 | OE2 | GLU | A | 353 | 11.321 | 21.075 | 65.637 | 1.00 | 40.80 | O |
| ATOM | 1446 | N | ALA | A | 354 | 10.334 | 15.935 | 63.223 | 1.00 | 29.90 | N |
| ATOM | 1447 | CA | ALA | A | 354 | 11.191 | 14.861 | 63.716 | 1.00 | 29.96 | C |
| ATOM | 1448 | C | ALA | A | 354 | 11.871 | 14.191 | 62.531 | 1.00 | 29.90 | C |
| ATOM | 1449 | O | ALA | A | 354 | 13.064 | 13.904 | 62.570 | 1.00 | 31.40 | O |
| ATOM | 1450 | CB | ALA | A | 354 | 10.367 | 13.843 | 64.491 | 1.00 | 30.18 | C |
| ATOM | 1451 | N | ILE | A | 355 | 11.100 | 13.940 | 61.478 | 1.00 | 29.82 | N |
| ATOM | 1452 | CA | ILE | A | 355 | 11.638 | 13.314 | 60.274 | 1.00 | 28.57 | C |
| ATOM | 1453 | C | ILE | A | 355 | 12.687 | 14.220 | 59.628 | 1.00 | 28.03 | C |
| ATOM | 1454 | O | ILE | A | 355 | 13.754 | 13.756 | 59.234 | 1.00 | 27.72 | O |
| ATOM | 1455 | CB | ILE | A | 355 | 10.514 | 13.022 | 59.259 | 1.00 | 29.38 | C |
| ATOM | 1456 | CG1 | ILE | A | 355 | 9.516 | 12.036 | 59.872 | 1.00 | 30.07 | C |
| ATOM | 1457 | CG2 | ILE | A | 355 | 11.101 | 12.458 | 57.964 | 1.00 | 30.16 | C |
| ATOM | 1458 | CD1 | ILE | A | 355 | 8.251 | 11.849 | 59.054 | 1.00 | 30.83 | C |
| ATOM | 1459 | N | GLN | A | 356 | 12.398 | 15.515 | 59.534 | 1.00 | 27.45 | N |
| ATOM | 1460 | CA | GLN | A | 356 | 13.345 | 16.444 | 58.925 | 1.00 | 28.48 | C |
| ATOM | 1461 | C | GLN | A | 356 | 14.621 | 16.566 | 59.754 | 1.00 | 29.24 | C |
| ATOM | 1462 | O | GLN | A | 356 | 15.719 | 16.622 | 59.202 | 1.00 | 27.50 | O |
| ATOM | 1463 | CB | GLN | A | 356 | 12.718 | 17.833 | 58.739 | 1.00 | 28.93 | C |
| ATOM | 1464 | CG | GLN | A | 356 | 13.536 | 18.753 | 57.823 | 1.00 | 29.68 | C |
| ATOM | 1465 | CD | GLN | A | 356 | 13.064 | 20.198 | 57.844 | 1.00 | 31.36 | C |
| ATOM | 1466 | OE1 | GLN | A | 356 | 12.996 | 20.823 | 58.903 | 1.00 | 31.51 | O |
| ATOM | 1467 | NE2 | GLN | A | 356 | 12.747 | 20.742 | 56.667 | 1.00 | 30.04 | N |
| ATOM | 1468 | N | ASP | A | 357 | 14.482 | 16.613 | 61.078 | 1.00 | 29.52 | N |
| ATOM | 1469 | CA | ASP | A | 357 | 15.656 | 16.724 | 61.945 | 1.00 | 30.44 | C |
| ATOM | 1470 | C | ASP | A | 357 | 16.610 | 15.550 | 61.739 | 1.00 | 29.70 | C |
| ATOM | 1471 | O | ASP | A | 357 | 17.827 | 15.729 | 61.727 | 1.00 | 30.37 | O |
| ATOM | 1472 | CB | ASP | A | 357 | 15.244 | 16.791 | 63.423 | 1.00 | 32.83 | C |
| ATOM | 1473 | CG | ASP | A | 357 | 14.665 | 18.141 | 63.812 | 1.00 | 34.76 | C |
| ATOM | 1474 | OD1 | ASP | A | 357 | 14.821 | 19.110 | 63.040 | 1.00 | 36.63 | O |
| ATOM | 1475 | OD2 | ASP | A | 357 | 14.065 | 18.236 | 64.905 | 1.00 | 36.89 | O |
| ATOM | 1476 | N | ARG | A | 358 | 16.059 | 14.351 | 61.577 | 1.00 | 30.08 | N |
| ATOM | 1477 | CA | ARG | A | 358 | 16.887 | 13.167 | 61.368 | 1.00 | 30.11 | C |
| ATOM | 1478 | C | ARG | A | 358 | 17.689 | 13.309 | 60.073 | 1.00 | 29.92 | C |
| ATOM | 1479 | O | ARG | A | 358 | 18.842 | 12.880 | 59.996 | 1.00 | 29.08 | O |
| ATOM | 1480 | CB | ARG | A | 358 | 16.014 | 11.906 | 61.323 | 1.00 | 31.17 | C |
| ATOM | 1481 | CG | ARG | A | 358 | 16.796 | 10.608 | 61.149 | 1.00 | 33.90 | C |
| ATOM | 1482 | CD | ARG | A | 358 | 15.919 | 9.382 | 61.402 | 1.00 | 36.03 | C |
| ATOM | 1483 | NE | ARG | A | 358 | 14.788 | 9.299 | 60.479 | 1.00 | 38.00 | N |
| ATOM | 1484 | CZ | ARG | A | 358 | 13.851 | 8.358 | 60.533 | 1.00 | 39.39 | C |
| ATOM | 1485 | NH1 | ARG | A | 358 | 13.909 | 7.417 | 61.469 | 1.00 | 39.22 | N |
| ATOM | 1486 | NH2 | ARG | A | 358 | 12.858 | 8.353 | 59.653 | 1.00 | 39.21 | N |
| ATOM | 1487 | N | LEU | A | 359 | 17.074 | 13.919 | 59.061 | 1.00 | 28.41 | N |
| ATOM | 1488 | CA | LEU | A | 359 | 17.735 | 14.128 | 57.776 | 1.00 | 27.76 | C |
| ATOM | 1489 | C | LEU | A | 359 | 18.757 | 15.255 | 57.890 | 1.00 | 27.89 | C |
| ATOM | 1490 | O | LEU | A | 359 | 19.853 | 15.171 | 57.338 | 1.00 | 27.13 | O |
| ATOM | 1491 | CB | LEU | A | 359 | 16.704 | 14.482 | 56.697 | 1.00 | 27.85 | C |
| ATOM | 1492 | CG | LEU | A | 359 | 15.646 | 13.421 | 56.384 | 1.00 | 27.95 | C |
| ATOM | 1493 | CD1 | LEU | A | 359 | 14.593 | 13.994 | 55.448 | 1.00 | 28.15 | C |
| ATOM | 1494 | CD2 | LEU | A | 359 | 16.310 | 12.210 | 55.758 | 1.00 | 28.81 | C |
| ATOM | 1495 | N | SER | A | 360 | 18.393 | 16.312 | 58.610 | 1.00 | 28.62 | N |
| ATOM | 1496 | CA | SER | A | 360 | 19.288 | 17.448 | 58.790 | 1.00 | 30.04 | C |
| ATOM | 1497 | C | SER | A | 360 | 20.540 | 17.046 | 59.561 | 1.00 | 30.70 | C |
| ATOM | 1498 | O | SER | A | 360 | 21.647 | 17.454 | 59.212 | 1.00 | 31.03 | O |
| ATOM | 1499 | CB | SER | A | 360 | 18.573 | 18.578 | 59.534 | 1.00 | 32.12 | C |
| ATOM | 1500 | OG | SER | A | 360 | 17.496 | 19.084 | 58.765 | 1.00 | 36.13 | O |
| ATOM | 1501 | N | ASN | A | 361 | 20.367 | 16.251 | 60.613 | 1.00 | 31.34 | N |
| ATOM | 1502 | CA | ASN | A | 361 | 21.513 | 15.816 | 61.405 | 1.00 | 31.58 | C |
| ATOM | 1503 | C | ASN | A | 361 | 22.417 | 14.921 | 60.570 | 1.00 | 30.77 | C |
| ATOM | 1504 | O | ASN | A | 361 | 23.637 | 14.935 | 60.728 | 1.00 | 31.06 | O |
| ATOM | 1505 | CB | ASN | A | 361 | 21.055 | 15.083 | 62.667 | 1.00 | 34.28 | C |
| ATOM | 1506 | CG | ASN | A | 361 | 20.328 | 15.998 | 63.637 | 1.00 | 37.26 | C |
| ATOM | 1507 | OD1 | ASN | A | 361 | 20.736 | 17.139 | 63.854 | 1.00 | 39.61 | O |
| ATOM | 1508 | ND2 | ASN | A | 361 | 19.252 | 15.497 | 64.234 | 1.00 | 39.64 | N |
| ATOM | 1509 | N | THR | A | 362 | 21.815 | 14.146 | 59.674 | 1.00 | 29.26 | N |
| ATOM | 1510 | CA | THR | A | 362 | 22.583 | 13.270 | 58.800 | 1.00 | 28.14 | C |
| ATOM | 1511 | C | THR | A | 362 | 23.419 | 14.135 | 57.863 | 1.00 | 27.56 | C |
| ATOM | 1512 | O | THR | A | 362 | 24.607 | 13.879 | 57.654 | 1.00 | 27.15 | O |
| ATOM | 1513 | CB | THR | A | 362 | 21.654 | 12.371 | 57.956 | 1.00 | 28.47 | C |
| ATOM | 1514 | OG1 | THR | A | 362 | 20.923 | 11.495 | 58.823 | 1.00 | 28.00 | O |
| ATOM | 1515 | CG2 | THR | A | 362 | 22.461 | 11.548 | 56.955 | 1.00 | 27.60 | C |
| ATOM | 1516 | N | LEU | A | 363 | 22.795 | 15.167 | 57.301 | 1.00 | 26.97 | N |
| ATOM | 1517 | CA | LEU | A | 363 | 23.493 | 16.064 | 56.388 | 1.00 | 27.40 | C |
| ATOM | 1518 | C | LEU | A | 363 | 24.623 | 16.798 | 57.100 | 1.00 | 28.23 | C |
| ATOM | 1519 | O | LEU | A | 363 | 25.736 | 16.884 | 56.588 | 1.00 | 27.96 | O |
| ATOM | 1520 | CB | LEU | A | 363 | 22.519 | 17.089 | 55.782 | 1.00 | 26.59 | C |
| ATOM | 1521 | CG | LEU | A | 363 | 23.153 | 18.156 | 54.882 | 1.00 | 26.54 | C |
| ATOM | 1522 | CD1 | LEU | A | 363 | 23.829 | 17.495 | 53.687 | 1.00 | 26.43 | C |
| ATOM | 1523 | CD2 | LEU | A | 363 | 22.090 | 19.142 | 54.417 | 1.00 | 26.28 | C |
| ATOM | 1524 | N | GLN | A | 364 | 24.340 | 17.325 | 58.286 | 1.00 | 29.48 | N |
| ATOM | 1525 | CA | GLN | A | 364 | 25.360 | 18.054 | 59.029 | 1.00 | 31.77 | C |
| ATOM | 1526 | C | GLN | A | 364 | 26.530 | 17.140 | 59.399 | 1.00 | 30.91 | C |
| ATOM | 1527 | O | GLN | A | 364 | 27.691 | 17.539 | 59.307 | 1.00 | 30.91 | O |
| ATOM | 1528 | CB | GLN | A | 364 | 24.747 | 18.681 | 60.283 | 1.00 | 33.97 | C |
| ATOM | 1529 | CG | GLN | A | 364 | 25.579 | 19.812 | 60.870 | 1.00 | 39.97 | C |
| ATOM | 1530 | CD | GLN | A | 364 | 24.749 | 20.793 | 61.681 | 1.00 | 41.73 | C |
| ATOM | 1531 | OE1 | GLN | A | 364 | 25.270 | 21.785 | 62.190 | 1.00 | 45.56 | O |
| ATOM | 1532 | NE2 | GLN | A | 364 | 23.452 | 20.523 | 61.800 | 1.00 | 43.48 | N |
| ATOM | 1533 | N | THR | A | 365 | 26.224 | 15.910 | 59.799 | 1.00 | 30.38 | N |
| ATOM | 1534 | CA | THR | A | 365 | 27.263 | 14.956 | 60.176 | 1.00 | 30.54 | C |
| ATOM | 1535 | C | THR | A | 365 | 28.099 | 14.561 | 58.965 | 1.00 | 29.67 | C |
| ATOM | 1536 | O | THR | A | 365 | 29.319 | 14.454 | 59.054 | 1.00 | 30.84 | O |
| ATOM | 1537 | CB | THR | A | 365 | 26.658 | 13.687 | 60.802 | 1.00 | 30.66 | C |
| ATOM | 1538 | OG1 | THR | A | 365 | 25.883 | 14.045 | 61.952 | 1.00 | 32.31 | O |
| ATOM | 1539 | CG2 | THR | A | 365 | 27.759 | 12.728 | 61.225 | 1.00 | 31.16 | C |
| ATOM | 1540 | N | TYR | A | 366 | 27.437 | 14.348 | 57.832 | 1.00 | 29.11 | N |
| ATOM | 1541 | CA | TYR | A | 366 | 28.131 | 13.976 | 56.606 | 1.00 | 28.71 | C |
| ATOM | 1542 | C | TYR | A | 366 | 29.123 | 15.051 | 56.167 | 1.00 | 28.75 | C |
| ATOM | 1543 | O | TYR | A | 366 | 30.261 | 14.746 | 55.826 | 1.00 | 29.49 | O |
| ATOM | 1544 | CB | TYR | A | 366 | 27.122 | 13.708 | 55.476 | 1.00 | 27.69 | C |
| ATOM | 1545 | CG | TYR | A | 366 | 27.779 | 13.396 | 54.148 | 1.00 | 27.26 | C |
| ATOM | 1546 | CD1 | TYR | A | 366 | 28.234 | 14.421 | 53.313 | 1.00 | 27.40 | C |
| ATOM | 1547 | CD2 | TYR | A | 366 | 28.017 | 12.079 | 53.759 | 1.00 | 27.61 | C |
| ATOM | 1548 | CE1 | TYR | A | 366 | 28.912 | 14.144 | 52.130 | 1.00 | 28.23 | C |
| ATOM | 1549 | CE2 | TYR | A | 366 | 28.697 | 11.790 | 52.578 | 1.00 | 28.00 | C |
| ATOM | 1550 | CZ | TYR | A | 366 | 29.143 | 12.825 | 51.770 | 1.00 | 28.28 | C |
| ATOM | 1551 | OH | TYR | A | 366 | 29.838 | 12.546 | 50.615 | 1.00 | 28.34 | O |
| ATOM | 1552 | N | ILE | A | 367 | 28.692 | 16.310 | 56.174 | 1.00 | 30.76 | N |
| ATOM | 1553 | CA | ILE | A | 367 | 29.559 | 17.412 | 55.762 | 1.00 | 32.74 | C |
| ATOM | 1554 | C | ILE | A | 367 | 30.823 | 17.533 | 56.614 | 1.00 | 35.15 | C |
| ATOM | 1555 | O | ILE | A | 367 | 31.924 | 17.688 | 56.086 | 1.00 | 35.56 | O |
| ATOM | 1556 | CB | ILE | A | 367 | 28.805 | 18.763 | 55.807 | 1.00 | 32.16 | C |
| ATOM | 1557 | CG1 | ILE | A | 367 | 27.685 | 18.764 | 54.763 | 1.00 | 32.04 | C |
| ATOM | 1558 | CG2 | ILE | A | 367 | 29.769 | 19.915 | 55.535 | 1.00 | 32.16 | C |
| ATOM | 1559 | CD1 | ILE | A | 367 | 26.790 | 19.977 | 54.829 | 1.00 | 32.66 | C |
| ATOM | 1560 | N | ARG | A | 368 | 30.660 | 17.465 | 57.930 | 1.00 | 38.07 | N |
| ATOM | 1561 | CA | ARG | A | 368 | 31.794 | 17.582 | 58.842 | 1.00 | 41.56 | C |
| ATOM | 1562 | C | ARG | A | 368 | 32.749 | 16.406 | 58.711 | 1.00 | 43.08 | C |
| ATOM | 1563 | O | ARG | A | 368 | 33.963 | 16.558 | 58.845 | 1.00 | 43.32 | O |
| ATOM | 1564 | CB | ARG | A | 368 | 31.309 | 17.652 | 60.289 | 1.00 | 43.01 | C |
| ATOM | 1565 | CG | ARG | A | 368 | 30.469 | 18.868 | 60.627 | 1.00 | 46.92 | C |
| ATOM | 1566 | CD | ARG | A | 368 | 30.023 | 18.814 | 62.081 | 1.00 | 50.17 | C |
| ATOM | 1567 | NE | ARG | A | 368 | 29.222 | 17.621 | 62.348 | 1.00 | 53.52 | N |
| ATOM | 1568 | CZ | ARG | A | 368 | 28.703 | 17.315 | 63.531 | 1.00 | 54.12 | C |
| ATOM | 1569 | NH1 | ARG | A | 368 | 28.901 | 18.117 | 64.570 | 1.00 | 55.63 | N |
| ATOM | 1570 | NH2 | ARG | A | 368 | 27.983 | 16.210 | 63.676 | 1.00 | 55.17 | N |
| ATOM | 1571 | N | CYS | A | 369 | 32.187 | 15.234 | 58.440 | 1.00 | 44.44 | N |
| ATOM | 1572 | CA | CYS | A | 369 | 32.964 | 14.011 | 58.333 | 1.00 | 46.85 | C |
| ATOM | 1573 | C | CYS | A | 369 | 33.501 | 13.644 | 56.949 | 1.00 | 46.74 | C |
| ATOM | 1574 | O | CYS | A | 369 | 34.641 | 13.198 | 56.828 | 1.00 | 46.26 | O |
| ATOM | 1575 | CB | CYS | A | 369 | 32.128 | 12.848 | 58.881 | 1.00 | 48.76 | C |
| ATOM | 1576 | SG | CYS | A | 369 | 32.925 | 11.238 | 58.816 | 1.00 | 56.08 | S |
| ATOM | 1577 | N | ARG | A | 370 | 32.700 | 13.841 | 55.905 | 1.00 | 47.07 | N |
| ATOM | 1578 | CA | ARG | A | 370 | 33.123 | 13.457 | 54.558 | 1.00 | 47.21 | C |
| ATOM | 1579 | C | ARG | A | 370 | 33.451 | 14.563 | 53.559 | 1.00 | 47.24 | C |
| ATOM | 1580 | O | ARG | A | 370 | 34.058 | 14.292 | 52.520 | 1.00 | 46.93 | O |
| ATOM | 1581 | CB | ARG | A | 370 | 32.068 | 12.533 | 53.940 | 1.00 | 47.76 | C |
| ATOM | 1582 | CG | ARG | A | 370 | 31.827 | 11.248 | 54.719 | 1.00 | 49.65 | C |
| ATOM | 1583 | CD | ARG | A | 370 | 33.034 | 10.323 | 54.660 | 1.00 | 51.30 | C |
| ATOM | 1584 | NE | ARG | A | 370 | 32.881 | 9.160 | 55.532 | 1.00 | 52.96 | N |
| ATOM | 1585 | CZ | ARG | A | 370 | 31.913 | 8.254 | 55.420 | 1.00 | 53.67 | C |
| ATOM | 1586 | NH1 | ARG | A | 370 | 30.999 | 8.367 | 54.465 | 1.00 | 54.23 | N |
| ATOM | 1587 | NH2 | ARG | A | 370 | 31.857 | 7.236 | 56.268 | 1.00 | 54.08 | N |
| ATOM | 1588 | N | HIS | A | 371 | 33.059 | 15.799 | 53.845 | 1.00 | 46.83 | N |
| ATOM | 1589 | CA | HIS | A | 371 | 33.340 | 16.879 | 52.908 | 1.00 | 47.01 | C |
| ATOM | 1590 | C | HIS | A | 371 | 34.670 | 17.554 | 53.217 | 1.00 | 47.99 | C |
| ATOM | 1591 | O | HIS | A | 371 | 34.809 | 18.227 | 54.237 | 1.00 | 46.99 | O |
| ATOM | 1592 | CB | HIS | A | 371 | 32.225 | 17.924 | 52.927 | 1.00 | 45.11 | C |
| ATOM | 1593 | CG | HIS | A | 371 | 32.126 | 18.713 | 51.659 | 1.00 | 44.21 | C |
| ATOM | 1594 | ND1 | HIS | A | 371 | 31.086 | 18.559 | 50.768 | 1.00 | 43.28 | N |
| ATOM | 1595 | CD2 | HIS | A | 371 | 32.959 | 19.629 | 51.111 | 1.00 | 43.81 | C |
| ATOM | 1596 | CE1 | HIS | A | 371 | 31.282 | 19.346 | 49.725 | 1.00 | 43.79 | C |
| ATOM | 1597 | NE2 | HIS | A | 371 | 32.412 | 20.005 | 49.907 | 1.00 | 43.86 | N |
| ATOM | 1598 | N | PRO | A | 372 | 35.665 | 17.383 | 52.331 | 1.00 | 49.67 | N |
| ATOM | 1599 | CA | PRO | A | 372 | 36.998 | 17.972 | 52.497 | 1.00 | 51.25 | C |
| ATOM | 1600 | C | PRO | A | 372 | 37.027 | 19.480 | 52.256 | 1.00 | 52.68 | C |
| ATOM | 1601 | O | PRO | A | 372 | 36.223 | 20.013 | 51.489 | 1.00 | 52.39 | O |
| ATOM | 1602 | CB | PRO | A | 372 | 37.833 | 17.208 | 51.476 | 1.00 | 51.31 | C |
| ATOM | 1603 | CG | PRO | A | 372 | 36.853 | 16.991 | 50.366 | 1.00 | 51.05 | C |
| ATOM | 1604 | CD | PRO | A | 372 | 35.613 | 16.549 | 51.115 | 1.00 | 50.21 | C |
| ATOM | 1605 | N | PRO | A | 373 | 37.961 | 20.187 | 52.914 | 1.00 | 54.03 | N |
| ATOM | 1606 | CA | PRO | A | 373 | 38.107 | 21.640 | 52.777 | 1.00 | 55.28 | C |
| ATOM | 1607 | C | PRO | A | 373 | 38.693 | 22.028 | 51.420 | 1.00 | 56.14 | C |
| ATOM | 1608 | O | PRO | A | 373 | 39.284 | 21.197 | 50.731 | 1.00 | 56.44 | O |
| ATOM | 1609 | CB | PRO | A | 373 | 39.036 | 21.998 | 53.932 | 1.00 | 55.37 | C |
| ATOM | 1610 | CG | PRO | A | 373 | 39.925 | 20.793 | 54.011 | 1.00 | 55.24 | C |
| ATOM | 1611 | CD | PRO | A | 373 | 38.934 | 19.653 | 53.885 | 1.00 | 54.59 | C |
| ATOM | 1612 | N | PRO | A | 374 | 38.535 | 23.299 | 51.017 | 1.00 | 56.81 | N |
| ATOM | 1613 | CA | PRO | A | 374 | 37.848 | 24.368 | 51.750 | 1.00 | 57.49 | C |
| ATOM | 1614 | C | PRO | A | 374 | 36.324 | 24.301 | 51.617 | 1.00 | 57.83 | C |
| ATOM | 1615 | O | PRO | A | 374 | 35.642 | 24.299 | 52.664 | 1.00 | 58.58 | O |
| ATOM | 1616 | CB | PRO | A | 374 | 38.431 | 25.631 | 51.127 | 1.00 | 57.24 | C |
| ATOM | 1617 | CG | PRO | A | 374 | 38.601 | 25.226 | 49.698 | 1.00 | 57.37 | C |
| ATOM | 1618 | CD | PRO | A | 374 | 39.194 | 23.834 | 49.811 | 1.00 | 57.05 | C |
| ATOM | 1619 | N | LEU | A | 378 | 30.279 | 26.156 | 57.018 | 1.00 | 49.88 | N |
| ATOM | 1620 | CA | LEU | A | 378 | 29.679 | 27.221 | 56.220 | 1.00 | 45.66 | C |
| ATOM | 1621 | C | LEU | A | 378 | 28.825 | 26.586 | 55.127 | 1.00 | 41.60 | C |
| ATOM | 1622 | O | LEU | A | 378 | 27.802 | 27.138 | 54.723 | 1.00 | 38.14 | O |
| ATOM | 1623 | CB | LEU | A | 378 | 30.769 | 28.092 | 55.590 | 1.00 | 53.84 | C |
| ATOM | 1624 | CG | LEU | A | 378 | 30.382 | 29.273 | 54.702 | 1.00 | 57.56 | C |
| ATOM | 1625 | CD1 | LEU | A | 378 | 29.709 | 30.349 | 55.545 | 1.00 | 59.64 | C |
| ATOM | 1626 | CD2 | LEU | A | 378 | 31.634 | 29.822 | 54.017 | 1.00 | 59.59 | C |
| ATOM | 1627 | N | LEU | A | 379 | 29.370 | 25.200 | 54.660 | 1.00 | 35.28 | N |
| ATOM | 1628 | CA | LEU | A | 379 | 28.529 | 24.615 | 53.626 | 1.00 | 33.21 | C |
| ATOM | 1629 | C | LEU | A | 379 | 27.095 | 24.355 | 54.080 | 1.00 | 32.24 | C |
| ATOM | 1630 | O | LEU | A | 379 | 26.157 | 24.594 | 53.325 | 1.00 | 31.09 | O |
| ATOM | 1631 | CB | LEU | A | 379 | 29.151 | 23.309 | 53.121 | 1.00 | 33.21 | C |
| ATOM | 1632 | CG | LEU | A | 379 | 28.379 | 22.603 | 52.003 | 1.00 | 31.83 | C |
| ATOM | 1633 | CD1 | LEU | A | 379 | 28.301 | 23.508 | 50.783 | 1.00 | 33.04 | C |
| ATOM | 1634 | CD2 | LEU | A | 379 | 29.066 | 21.292 | 51.651 | 1.00 | 32.12 | C |
| ATOM | 1635 | N | TYR | A | 380 | 26.917 | 23.869 | 55.304 | 1.00 | 31.72 | N |
| ATOM | 1636 | CA | TYR | A | 380 | 25.572 | 23.588 | 55.792 | 1.00 | 32.34 | C |
| ATOM | 1637 | C | TYR | A | 380 | 24.717 | 24.852 | 55.780 | 1.00 | 32.63 | C |
| ATOM | 1638 | O | TYR | A | 380 | 23.562 | 24.833 | 55.339 | 1.00 | 31.56 | O |
| ATOM | 1639 | CB | TYR | A | 380 | 25.611 | 23.008 | 57.208 | 1.00 | 33.03 | C |
| ATOM | 1640 | CG | TYR | A | 380 | 24.239 | 22.659 | 57.743 | 1.00 | 34.66 | C |
| ATOM | 1641 | CD1 | TYR | A | 380 | 23.486 | 21.635 | 57.169 | 1.00 | 35.98 | C |
| ATOM | 1642 | CD2 | TYR | A | 380 | 23.680 | 23.373 | 58.800 | 1.00 | 35.42 | C |
| ATOM | 1643 | CE1 | TYR | A | 380 | 22.209 | 21.333 | 57.636 | 1.00 | 36.98 | C |
| ATOM | 1644 | CE2 | TYR | A | 380 | 22.410 | 23.080 | 59.274 | 1.00 | 36.43 | C |
| ATOM | 1645 | CZ | TYR | A | 380 | 21.679 | 22.060 | 58.688 | 1.00 | 37.93 | C |
| ATOM | 1646 | OH | TYR | A | 380 | 20.420 | 21.770 | 59.154 | 1.00 | 38.77 | O |
| ATOM | 1647 | N | ALA | A | 381 | 25.288 | 25.950 | 56.266 | 1.00 | 32.03 | N |
| ATOM | 1648 | CA | ALA | A | 381 | 24.578 | 27.223 | 56.304 | 1.00 | 32.04 | C |
| ATOM | 1649 | C | ALA | A | 381 | 24.190 | 27.683 | 54.902 | 1.00 | 31.59 | C |
| ATOM | 1650 | O | ALA | A | 381 | 23.084 | 28.187 | 54.693 | 1.00 | 32.20 | O |
| ATOM | 1651 | CB | ALA | A | 381 | 25.443 | 28.287 | 56.981 | 1.00 | 32.84 | C |
| ATOM | 1652 | N | LYS | A | 382 | 25.101 | 27.515 | 53.948 | 1.00 | 30.09 | N |
| ATOM | 1653 | CA | LYS | A | 382 | 24.849 | 27.916 | 52.570 | 1.00 | 30.96 | C |
| ATOM | 1654 | C | LYS | A | 382 | 23.739 | 27.083 | 51.943 | 1.00 | 30.08 | C |
| ATOM | 1655 | O | LYS | A | 382 | 22.989 | 27.575 | 51.101 | 1.00 | 30.60 | O |
| ATOM | 1656 | CB | LYS | A | 382 | 26.121 | 27.781 | 51.731 | 1.00 | 31.98 | C |
| ATOM | 1657 | CG | LYS | A | 382 | 27.223 | 28.757 | 52.109 | 1.00 | 34.76 | C |
| ATOM | 1658 | CD | LYS | A | 382 | 28.458 | 28.545 | 51.254 | 1.00 | 38.05 | C |
| ATOM | 1659 | CE | LYS | A | 382 | 29.559 | 29.526 | 51.615 | 1.00 | 39.31 | C |
| ATOM | 1660 | NZ | LYS | A | 382 | 30.806 | 29.245 | 50.845 | 1.00 | 41.47 | N |
| ATOM | 1661 | N | MET | A | 383 | 23.648 | 25.819 | 52.345 | 1.00 | 29.65 | N |
| ATOM | 1662 | CA | MET | A | 383 | 22.621 | 24.923 | 51.821 | 1.00 | 29.41 | C |
| ATOM | 1663 | C | MET | A | 383 | 21.253 | 25.286 | 52.389 | 1.00 | 29.82 | C |
| ATOM | 1664 | O | MET | A | 383 | 20.250 | 25.271 | 51.677 | 1.00 | 29.12 | O |
| ATOM | 1665 | CB | MET | A | 383 | 22.958 | 23.468 | 52.165 | 1.00 | 28.17 | C |
| ATOM | 1666 | CG | MET | A | 383 | 24.130 | 22.908 | 51.381 | 1.00 | 28.12 | C |
| ATOM | 1667 | SD | MET | A | 383 | 24.510 | 21.186 | 51.776 | 1.00 | 28.48 | S |
| ATOM | 1668 | CE | MET | A | 383 | 23.099 | 20.338 | 51.048 | 1.00 | 28.89 | C |
| ATOM | 1669 | N | ILE | A | 384 | 21.215 | 25.612 | 53.676 | 1.00 | 30.76 | N |
| ATOM | 1670 | CA | ILE | A | 384 | 19.960 | 25.983 | 54.319 | 1.00 | 32.84 | C |
| ATOM | 1671 | C | ILE | A | 384 | 19.422 | 27.271 | 53.701 | 1.00 | 32.96 | C |
| ATOM | 1672 | O | ILE | A | 384 | 18.208 | 27.458 | 53.594 | 1.00 | 32.83 | O |
| ATOM | 1673 | CB | ILE | A | 384 | 20.149 | 26.186 | 55.842 | 1.00 | 34.53 | C |
| ATOM | 1674 | CG1 | ILE | A | 384 | 20.651 | 24.889 | 56.482 | 1.00 | 36.66 | C |
| ATOM | 1675 | CG2 | ILE | A | 384 | 18.834 | 26.610 | 56.482 | 1.00 | 36.24 | C |
| ATOM | 1676 | CD1 | ILE | A | 384 | 19.744 | 23.691 | 56.257 | 1.00 | 37.66 | C |
| ATOM | 1677 | N | GLN | A | 385 | 20.328 | 28.153 | 53.287 | 1.00 | 32.82 | N |
| ATOM | 1678 | CA | GLN | A | 385 | 19.931 | 29.412 | 52.669 | 1.00 | 33.03 | C |
| ATOM | 1679 | C | GLN | A | 385 | 19.288 | 29.174 | 51.303 | 1.00 | 32.26 | C |
| ATOM | 1680 | O | GLN | A | 385 | 18.382 | 29.905 | 50.901 | 1.00 | 30.38 | O |
| ATOM | 1681 | CB | GLN | A | 385 | 21.136 | 30.342 | 52.515 | 1.00 | 35.19 | C |
| ATOM | 1682 | CG | GLN | A | 385 | 20.839 | 31.588 | 51.692 | 1.00 | 39.54 | C |
| ATOM | 1683 | CD | GLN | A | 385 | 19.705 | 32.421 | 52.270 | 1.00 | 41.95 | C |
| ATOM | 1684 | OE1 | GLN | A | 385 | 19.024 | 33.151 | 51.545 | 1.00 | 44.07 | O |
| ATOM | 1685 | NE2 | GLN | A | 385 | 19.504 | 32.324 | 53.579 | 1.00 | 42.90 | N |
| ATOM | 1686 | N | LYS | A | 386 | 19.756 | 28.152 | 50.591 | 1.00 | 30.96 | N |
| ATOM | 1687 | CA | LYS | A | 386 | 19.197 | 27.840 | 49.282 | 1.00 | 30.88 | C |
| ATOM | 1688 | C | LYS | A | 386 | 17.748 | 27.415 | 49.447 | 1.00 | 29.98 | C |
| ATOM | 1689 | O | LYS | A | 386 | 16.927 | 27.635 | 48.558 | 1.00 | 29.72 | O |
| ATOM | 1690 | CB | LYS | A | 386 | 19.985 | 26.719 | 48.601 | 1.00 | 32.29 | C |
| ATOM | 1691 | CG | LYS | A | 386 | 21.430 | 27.064 | 48.310 | 1.00 | 35.09 | C |
| ATOM | 1692 | CD | LYS | A | 386 | 21.539 | 28.305 | 47.453 | 1.00 | 36.92 | C |
| ATOM | 1693 | CE | LYS | A | 386 | 22.997 | 28.643 | 47.170 | 1.00 | 39.17 | C |
| ATOM | 1694 | NZ | LYS | A | 386 | 23.133 | 30.008 | 46.589 | 1.00 | 40.63 | N |
| ATOM | 1695 | N | LEU | A | 387 | 17.433 | 26.804 | 50.583 | 1.00 | 29.25 | N |
| ATOM | 1696 | CA | LEU | A | 387 | 16.064 | 26.373 | 50.833 | 1.00 | 29.50 | C |
| ATOM | 1697 | C | LEU | A | 387 | 15.172 | 27.604 | 50.982 | 1.00 | 29.30 | C |
| ATOM | 1698 | O | LEU | A | 387 | 14.014 | 27.594 | 50.572 | 1.00 | 27.98 | O |
| ATOM | 1699 | CB | LEU | A | 387 | 15.988 | 25.503 | 52.091 | 1.00 | 30.49 | C |
| ATOM | 1700 | CG | LEU | A | 387 | 16.625 | 24.111 | 51.980 | 1.00 | 31.01 | C |
| ATOM | 1701 | CD1 | LEU | A | 387 | 16.443 | 23.363 | 53.289 | 1.00 | 33.01 | C |
| ATOM | 1702 | CD2 | LEU | A | 387 | 15.985 | 23.339 | 50.839 | 1.00 | 31.27 | C |
| ATOM | 1703 | N | ALA | A | 388 | 15.714 | 28.667 | 51.566 | 1.00 | 28.72 | N |
| ATOM | 1704 | CA | ALA | A | 388 | 14.952 | 29.903 | 51.735 | 1.00 | 29.54 | C |
| ATOM | 1705 | C | ALA | A | 388 | 14.757 | 30.557 | 50.367 | 1.00 | 29.67 | C |
| ATOM | 1706 | O | ALA | A | 388 | 13.696 | 31.121 | 50.082 | 1.00 | 29.63 | O |
| ATOM | 1707 | CB | ALA | A | 388 | 15.687 | 30.856 | 52.679 | 1.00 | 30.01 | C |
| ATOM | 1708 | N | ASP | A | 389 | 15.786 | 30.479 | 49.524 | 1.00 | 29.62 | N |
| ATOM | 1709 | CA | ASP | A | 389 | 15.730 | 31.044 | 48.175 | 1.00 | 30.08 | C |
| ATOM | 1710 | C | ASP | A | 389 | 14.625 | 30.360 | 47.378 | 1.00 | 29.78 | C |
| ATOM | 1711 | O | ASP | A | 389 | 13.917 | 31.000 | 46.598 | 1.00 | 29.18 | O |
| ATOM | 1712 | CB | ASP | A | 389 | 17.059 | 30.833 | 47.445 | 1.00 | 31.12 | C |
| ATOM | 1713 | CG | ASP | A | 389 | 18.183 | 31.682 | 48.006 | 1.00 | 34.91 | C |
| ATOM | 1714 | OD1 | ASP | A | 389 | 19.352 | 31.423 | 47.643 | 1.00 | 36.63 | O |
| ATOM | 1715 | OD2 | ASP | A | 389 | 17.901 | 32.608 | 48.796 | 1.00 | 35.52 | O |
| ATOM | 1716 | N | LEU | A | 390 | 14.496 | 29.052 | 47.572 | 1.00 | 27.64 | N |
| ATOM | 1717 | CA | LEU | A | 390 | 13.482 | 28.262 | 46.881 | 1.00 | 28.50 | C |
| ATOM | 1718 | C | LEU | A | 390 | 12.067 | 28.730 | 47.223 | 1.00 | 27.80 | C |
| ATOM | 1719 | O | LEU | A | 390 | 11.187 | 28.740 | 46.360 | 1.00 | 26.98 | O |
| ATOM | 1720 | CB | LEU | A | 390 | 13.646 | 26.786 | 47.252 | 1.00 | 28.87 | C |
| ATOM | 1721 | CG | LEU | A | 390 | 14.130 | 25.761 | 46.221 | 1.00 | 31.08 | C |
| ATOM | 1722 | CD1 | LEU | A | 390 | 14.754 | 26.424 | 45.017 | 1.00 | 31.00 | C |
| ATOM | 1723 | CD2 | LEU | A | 390 | 15.101 | 24.810 | 46.902 | 1.00 | 31.75 | C |
| ATOM | 1724 | N | ARG | A | 391 | 11.849 | 29.109 | 48.481 | 1.00 | 27.87 | N |
| ATOM | 1725 | CA | ARG | A | 391 | 10.535 | 29.574 | 48.917 | 1.00 | 28.52 | C |
| ATOM | 1726 | C | ARG | A | 391 | 10.132 | 30.808 | 48.125 | 1.00 | 28.78 | C |
| ATOM | 1727 | O | ARG | A | 391 | 8.968 | 30.961 | 47.757 | 1.00 | 28.82 | O |
| ATOM | 1728 | CB | ARG | A | 391 | 10.536 | 29.919 | 50.415 | 1.00 | 30.35 | C |
| ATOM | 1729 | CG | ARG | A | 391 | 10.795 | 28.744 | 51.354 | 1.00 | 32.51 | C |
| ATOM | 1730 | CD | ARG | A | 391 | 9.743 | 27.658 | 51.208 | 1.00 | 34.99 | C |
| ATOM | 1731 | NE | ARG | A | 391 | 9.952 | 26.552 | 52.141 | 1.00 | 37.12 | N |
| ATOM | 1732 | CZ | ARG | A | 391 | 9.395 | 26.460 | 53.346 | 1.00 | 37.91 | C |
| ATOM | 1733 | NH1 | ARG | A | 391 | 8.580 | 27.411 | 53.783 | 1.00 | 38.94 | N |
| ATOM | 1734 | NH2 | ARG | A | 391 | 9.646 | 25.408 | 54.115 | 1.00 | 37.42 | N |
| ATOM | 1735 | N | SER | A | 392 | 11.094 | 31.690 | 47.865 | 1.00 | 28.76 | N |
| ATOM | 1736 | CA | SER | A | 392 | 10.811 | 32.908 | 47.114 | 1.00 | 29.36 | C |
| ATOM | 1737 | C | SER | A | 392 | 10.483 | 32.588 | 45.664 | 1.00 | 28.02 | C |
| ATOM | 1738 | O | SER | A | 392 | 9.577 | 33.178 | 45.082 | 1.00 | 28.38 | O |
| ATOM | 1739 | CB | SER | A | 392 | 11.997 | 33.866 | 47.185 | 1.00 | 31.21 | C |
| ATOM | 1740 | OG | SER | A | 392 | 12.192 | 34.305 | 48.518 | 1.00 | 37.19 | O |
| ATOM | 1741 | N | LEU | A | 393 | 11.219 | 31.648 | 45.081 | 1.00 | 26.23 | N |
| ATOM | 1742 | CA | LEU | A | 393 | 10.972 | 31.253 | 43.700 | 1.00 | 26.10 | C |
| ATOM | 1743 | C | LEU | A | 393 | 9.614 | 30.567 | 43.586 | 1.00 | 25.57 | C |
| ATOM | 1744 | O | LEU | A | 393 | 8.919 | 30.705 | 42.576 | 1.00 | 26.87 | O |
| ATOM | 1745 | CB | LEU | A | 393 | 12.081 | 30.309 | 43.216 | 1.00 | 26.02 | C |
| ATOM | 1746 | CG | LEU | A | 393 | 13.450 | 30.968 | 43.030 | 1.00 | 26.66 | C |
| ATOM | 1747 | CD1 | LEU | A | 393 | 14.536 | 29.905 | 42.878 | 1.00 | 28.52 | C |
| ATOM | 1748 | CD2 | LEU | A | 393 | 13.400 | 31.869 | 41.808 | 1.00 | 29.45 | C |
| ATOM | 1749 | N | ASN | A | 394 | 9.242 | 29.825 | 44.625 | 1.00 | 24.50 | N |
| ATOM | 1750 | CA | ASN | A | 394 | 7.964 | 29.122 | 44.656 | 1.00 | 26.07 | C |
| ATOM | 1751 | C | ASN | A | 394 | 6.855 | 30.167 | 44.570 | 1.00 | 27.28 | C |
| ATOM | 1752 | O | ASN | A | 394 | 5.929 | 30.055 | 43.764 | 1.00 | 26.29 | O |
| ATOM | 1753 | CB | ASN | A | 394 | 7.827 | 28.347 | 45.967 | 1.00 | 26.75 | C |
| ATOM | 1754 | CG | ASN | A | 394 | 6.646 | 27.397 | 45.968 | 1.00 | 28.26 | C |
| ATOM | 1755 | OD1 | ASN | A | 394 | 5.660 | 27.604 | 45.263 | 1.00 | 28.24 | O |
| ATOM | 1756 | ND2 | ASN | A | 394 | 6.736 | 26.352 | 46.779 | 1.00 | 28.79 | N |
| ATOM | 1757 | N | GLU | A | 395 | 6.966 | 31.188 | 45.413 | 1.00 | 28.62 | N |
| ATOM | 1758 | CA | GLU | A | 395 | 5.986 | 32.266 | 45.464 | 1.00 | 30.55 | C |
| ATOM | 1759 | C | GLU | A | 395 | 5.815 | 32.976 | 44.130 | 1.00 | 29.66 | C |
| ATOM | 1760 | O | GLU | A | 395 | 4.691 | 33.213 | 43.684 | 1.00 | 29.50 | O |
| ATOM | 1761 | CB | GLU | A | 395 | 6.385 | 33.280 | 46.536 | 1.00 | 33.44 | C |
| ATOM | 1762 | CG | GLU | A | 395 | 6.277 | 32.744 | 47.954 | 1.00 | 40.01 | C |
| ATOM | 1763 | CD | GLU | A | 395 | 4.838 | 32.481 | 48.366 | 1.00 | 44.38 | C |
| ATOM | 1764 | OE1 | GLU | A | 395 | 4.618 | 32.045 | 49.518 | 1.00 | 46.89 | O |
| ATOM | 1765 | OE2 | GLU | A | 395 | 3.924 | 32.713 | 47.540 | 1.00 | 46.99 | O |
| ATOM | 1766 | NC | GLU | A | 396 | 6.929 | 33.324 | 43.496 | 1.00 | 29.08 | N |
| ATOM | 1767 | CA | GLU | A | 396 | 6.871 | 34.013 | 42.217 | 1.00 | 28.78 | C |
| ATOM | 1768 | C | GLU | A | 396 | 6.280 | 33.102 | 41.148 | 1.00 | 28.20 | C |
| ATOM | 1769 | O | GLU | A | 396 | 5.486 | 33.545 | 40.317 | 1.00 | 27.96 | O |
| ATOM | 1770 | CB | GLU | A | 396 | 8.265 | 34.490 | 41.791 | 1.00 | 30.45 | C |
| ATOM | 1771 | CG | GLU | A | 396 | 8.276 | 35.254 | 40.465 | 1.00 | 30.29 | C |
| ATOM | 1772 | CD | GLU | A | 396 | 7.502 | 36.568 | 40.525 | 1.00 | 33.32 | C |
| ATOM | 1773 | OE1 | GLU | A | 396 | 7.098 | 37.068 | 39.452 | 1.00 | 32.46 | O |
| ATOM | 1774 | OE2 | GLU | A | 396 | 7.307 | 37.108 | 41.639 | 1.00 | 32.27 | O |
| ATOM | 1775 | N | HIS | A | 397 | 6.651 | 31.826 | 41.162 | 1.00 | 26.94 | N |
| ATOM | 1776 | CA | HIS | A | 397 | 6.104 | 30.919 | 40.162 | 1.00 | 27.05 | C |
| ATOM | 1777 | C | HIS | A | 397 | 4.583 | 30.835 | 40.295 | 1.00 | 27.50 | C |
| ATOM | 1778 | O | HIS | A | 397 | 3.866 | 30.834 | 39.294 | 1.00 | 27.05 | O |
| ATOM | 1779 | CB | HIS | A | 397 | 6.718 | 29.519 | 40.282 | 1.00 | 26.64 | C |
| ATOM | 1780 | CG | HIS | A | 397 | 6.058 | 28.507 | 39.400 | 1.00 | 26.04 | C |
| ATOM | 1781 | ND1 | HIS | A | 397 | 4.999 | 27.731 | 39.822 | 1.00 | 27.22 | N |
| ATOM | 1782 | CD2 | HIS | A | 397 | 6.227 | 28.228 | 38.086 | 1.00 | 26.36 | C |
| ATOM | 1783 | CE1 | HIS | A | 397 | 4.542 | 27.024 | 38.805 | 1.00 | 26.59 | C |
| ATOM | 1784 | NE2 | HIS | A | 397 | 5.268 | 27.308 | 37.740 | 1.00 | 26.30 | N |
| ATOM | 1785 | N | SER | A | 398 | 4.094 | 30.785 | 41.529 | 1.00 | 28.04 | N |
| ATOM | 1786 | CA | SER | A | 398 | 2.657 | 30.696 | 41.775 | 1.00 | 29.62 | C |
| ATOM | 1787 | C | SER | A | 398 | 1.921 | 31.901 | 41.195 | 1.00 | 29.87 | C |
| ATOM | 1788 | O | SER | A | 398 | 0.862 | 31.761 | 40.579 | 1.00 | 28.32 | O |
| ATOM | 1789 | CB | SER | A | 398 | 2.389 | 30.604 | 43.279 | 1.00 | 31.44 | C |
| ATOM | 1790 | OG | SER | A | 398 | 1.000 | 30.483 | 43.534 | 1.00 | 38.72 | O |
| ATOM | 1791 | N | LYS | A | 399 | 2.485 | 33.085 | 41.397 | 1.00 | 30.06 | N |
| ATOM | 1792 | CA | LYS | A | 399 | 1.882 | 34.313 | 40.885 | 1.00 | 31.61 | C |
| ATOM | 1793 | C | LYS | A | 399 | 1.807 | 34.283 | 39.363 | 1.00 | 30.91 | C |
| ATOM | 1794 | O | LYS | A | 399 | 0.790 | 34.651 | 38.771 | 1.00 | 30.21 | O |
| ATOM | 1795 | CB | LYS | A | 399 | 2.698 | 35.527 | 41.336 | 1.00 | 33.79 | C |
| ATOM | 1796 | CG | LYS | A | 399 | 2.754 | 35.693 | 42.842 | 1.00 | 38.63 | C |
| ATOM | 1797 | CD | LYS | A | 399 | 3.521 | 36.946 | 43.236 | 1.00 | 41.48 | C |
| ATOM | 1798 | CE | LYS | A | 399 | 3.571 | 37.101 | 44.750 | 1.00 | 43.57 | C |
| ATOM | 1799 | NZ | LYS | A | 399 | 4.340 | 38.313 | 45.155 | 1.00 | 44.90 | N |
| ATOM | 1800 | N | GLN | A | 400 | 2.886 | 33.834 | 38.731 | 1.00 | 29.20 | N |
| ATOM | 1801 | CA | GLN | A | 400 | 2.926 | 33.770 | 37.278 | 1.00 | 28.78 | C |
| ATOM | 1802 | C | GLN | A | 400 | 2.052 | 32.660 | 36.702 | 1.00 | 27.95 | C |
| ATOM | 1803 | O | GLN | A | 400 | 1.524 | 32.789 | 35.595 | 1.00 | 27.64 | O |
| ATOM | 1804 | CB | GLN | A | 400 | 4.374 | 33.637 | 36.802 | 1.00 | 28.49 | C |
| ATOM | 1805 | CG | GLN | A | 400 | 5.147 | 34.942 | 36.964 | 1.00 | 30.64 | C |
| ATOM | 1806 | CD | GLN | A | 400 | 6.483 | 34.940 | 36.256 | 1.00 | 31.34 | C |
| ATOM | 1807 | OE1 | GLN | A | 400 | 6.673 | 34.235 | 35.265 | 1.00 | 33.90 | O |
| ATOM | 1808 | NE2 | GLN | A | 400 | 7.414 | 35.751 | 36.749 | 1.00 | 31.02 | N |
| ATOM | 1809 | N | TYR | A | 401 | 1.894 | 31.571 | 37.446 | 1.00 | 26.56 | N |
| ATOM | 1810 | CA | TYR | A | 401 | 1.051 | 30.481 | 36.980 | 1.00 | 27.31 | C |
| ATOM | 1811 | C | TYR | A | 401 | −0.382 | 30.998 | 36.941 | 1.00 | 27.98 | C |
| ATOM | 1812 | O | TYR | A | 401 | −1.147 | 30.686 | 36.024 | 1.00 | 27.25 | O |
| ATOM | 1813 | CB | TYR | A | 401 | 1.127 | 29.285 | 37.931 | 1.00 | 27.75 | C |
| ATOM | 1814 | CG | TYR | A | 401 | 0.229 | 28.147 | 37.516 | 1.00 | 27.44 | C |
| ATOM | 1815 | CD1 | TYR | A | 401 | 0.600 | 27.281 | 36.489 | 1.00 | 28.38 | C |
| ATOM | 1816 | CD2 | TYR | A | 401 | −1.013 | 27.960 | 38.119 | 1.00 | 29.26 | C |
| ATOM | 1817 | CE1 | TYR | A | 401 | −0.242 | 26.260 | 36.068 | 1.00 | 28.43 | C |
| ATOM | 1818 | CE2 | TYR | A | 401 | −1.868 | 26.938 | 37.703 | 1.00 | 29.62 | C |
| ATOM | 1819 | CZ | TYR | A | 401 | −1.475 | 26.094 | 36.677 | 1.00 | 29.95 | C |
| ATOM | 1820 | OH | TYR | A | 401 | −2.319 | 25.089 | 36.252 | 1.00 | 30.37 | O |
| ATOM | 1821 | N | ARG | A | 402 | −0.742 | 31.790 | 37.948 | 1.00 | 29.27 | N |
| ATOM | 1822 | CA | ARG | A | 402 | −2.083 | 32.360 | 38.021 | 1.00 | 32.16 | C |
| ATOM | 1823 | C | ARG | A | 402 | −2.386 | 33.173 | 36.769 | 1.00 | 32.00 | C |
| ATOM | 1824 | O | ARG | A | 402 | −3.434 | 32.998 | 36.150 | 1.00 | 31.31 | O |
| ATOM | 1825 | CB | ARG | A | 402 | −2.220 | 33.251 | 39.256 | 1.00 | 36.10 | C |
| ATOM | 1826 | CG | ARG | A | 402 | −3.587 | 33.906 | 39.391 | 1.00 | 41.28 | C |
| ATOM | 1827 | CD | ARG | A | 402 | −3.710 | 34.730 | 40.669 | 1.00 | 45.66 | C |
| ATOM | 1828 | NE | ARG | A | 402 | −3.552 | 33.916 | 41.873 | 1.00 | 49.74 | N |
| ATOM | 1829 | CZ | ARG | A | 402 | −2.382 | 33.582 | 42.410 | 1.00 | 51.64 | C |
| ATOM | 1830 | NH1 | ARG | A | 402 | −1.249 | 33.996 | 41.856 | 1.00 | 52.67 | N |
| ATOM | 1831 | NH2 | ARG | A | 402 | −2.343 | 32.825 | 43.499 | 1.00 | 52.69 | N |
| ATOM | 1832 | N | CYS | A | 403 | −1.471 | 34.066 | 36.402 | 1.00 | 32.10 | N |
| ATOM | 1833 | CA | CYS | A | 403 | −1.645 | 34.895 | 35.210 | 1.00 | 33.04 | C |
| ATOM | 1834 | C | CYS | A | 403 | −1.781 | 34.014 | 33.976 | 1.00 | 32.14 | C |
| ATOM | 1835 | O | CYS | A | 403 | −2.620 | 34.257 | 33.106 | 1.00 | 30.55 | O |
| ATOM | 1836 | CB | CYS | A | 403 | −0.450 | 35.838 | 35.030 | 1.00 | 35.60 | C |
| ATOM | 1837 | SG | CYS | A | 403 | −0.253 | 36.492 | 33.340 | 1.00 | 44.46 | S |
| ATOM | 1838 | N | LEU | A | 404 | −0.950 | 32.980 | 33.911 | 1.00 | 30.59 | N |
| ATOM | 1839 | CA | LEU | A | 404 | −0.967 | 32.065 | 32.784 | 1.00 | 30.86 | C |
| ATOM | 1840 | C | LEU | A | 404 | −2.327 | 31.390 | 32.638 | 1.00 | 29.62 | C |
| ATOM | 1841 | O | LEU | A | 404 | −2.840 | 31.256 | 31.529 | 1.00 | 30.21 | O |
| ATOM | 1842 | CB | LEU | A | 404 | 0.130 | 31.008 | 32.955 | 1.00 | 32.55 | C |
| ATOM | 1843 | CG | LEU | A | 404 | 0.353 | 30.078 | 31.766 | 1.00 | 34.87 | C |
| ATOM | 1844 | CD1 | LEU | A | 404 | 0.840 | 30.895 | 30.580 | 1.00 | 36.26 | C |
| ATOM | 1845 | CD2 | LEU | A | 404 | 1.370 | 29.005 | 32.127 | 1.00 | 35.52 | C |
| ATOM | 1846 | N | SER | A | 405 | −2.918 | 30.987 | 33.760 | 1.00 | 29.30 | N |
| ATOM | 1847 | CA | SER | A | 405 | −4.212 | 30.309 | 33.749 | 1.00 | 29.83 | C |
| ATOM | 1848 | C | SER | A | 405 | −5.358 | 31.173 | 33.218 | 1.00 | 28.16 | C |
| ATOM | 1849 | O | SER | A | 405 | −6.423 | 30.651 | 32.885 | 1.00 | 28.54 | O |
| ATOM | 1850 | CB | SER | A | 405 | −4.563 | 29.802 | 35.153 | 1.00 | 31.58 | C |
| ATOM | 1851 | OG | SER | A | 405 | −4.841 | 30.873 | 36.040 | 1.00 | 34.11 | O |
| ATOM | 1852 | N | PHE | A | 406 | −5.147 | 32.484 | 33.145 | 1.00 | 25.99 | N |
| ATOM | 1853 | CA | PHE | A | 406 | −6.179 | 33.396 | 32.636 | 1.00 | 26.56 | C |
| ATOM | 1854 | C | PHE | A | 406 | −6.263 | 33.340 | 31.112 | 1.00 | 26.23 | C |
| ATOM | 1855 | O | PHE | A | 406 | −7.256 | 33.778 | 30.518 | 1.00 | 25.59 | O |
| ATOM | 1856 | CB | PHE | A | 406 | −5.868 | 34.842 | 33.042 | 1.00 | 26.14 | C |
| ATOM | 1857 | CG | PHE | A | 406 | −6.058 | 35.128 | 34.503 | 1.00 | 28.26 | C |
| ATOM | 1858 | CD1 | PHE | A | 406 | −5.386 | 36.196 | 35.099 | 1.00 | 29.88 | C |
| ATOM | 1859 | CD2 | PHE | A | 406 | −6.920 | 34.361 | 35.278 | 1.00 | 29.84 | C |
| ATOM | 1860 | CE1 | PHE | A | 406 | −5.570 | 36.494 | 36.446 | 1.00 | 30.77 | C |
| ATOM | 1861 | CE2 | PHE | A | 406 | −7.112 | 34.651 | 36.632 | 1.00 | 31.26 | C |
| ATOM | 1862 | CZ | PHE | A | 406 | −6.436 | 35.719 | 37.214 | 1.00 | 30.45 | C |
| ATOM | 1863 | N | GLN | A | 407 | −5.220 | 32.814 | 30.478 | 1.00 | 25.64 | N |
| ATOM | 1864 | CA | GLN | A | 407 | −5.189 | 32.748 | 29.019 | 1.00 | 25.17 | C |
| ATOM | 1865 | C | GLN | A | 407 | −6.155 | 31.687 | 28.500 | 1.00 | 25.33 | C |
| ATOM | 1866 | O | GLN | A | 407 | −6.086 | 30.524 | 28.903 | 1.00 | 24.86 | O |
| ATOM | 1867 | CB | GLN | A | 407 | −3.765 | 32.448 | 28.527 | 1.00 | 25.99 | C |
| ATOM | 1868 | CG | GLN | A | 407 | −3.571 | 32.694 | 27.030 | 1.00 | 26.23 | C |
| ATOM | 1869 | CD | GLN | A | 407 | −3.718 | 34.165 | 26.651 | 1.00 | 26.81 | C |
| ATOM | 1870 | OE1 | GLN | A | 407 | −4.087 | 34.494 | 25.520 | 1.00 | 28.94 | O |
| ATOM | 1871 | NE2 | GLN | A | 407 | −3.414 | 35.052 | 27.590 | 1.00 | 21.63 | N |
| ATOM | 1872 | N | PRO | A | 408 | −7.083 | 32.079 | 27.608 | 1.00 | 25.83 | N |
| ATOM | 1873 | CA | PRO | A | 408 | −8.052 | 31.124 | 27.058 | 1.00 | 27.42 | C |
| ATOM | 1874 | C | PRO | A | 408 | −7.384 | 29.913 | 26.398 | 1.00 | 29.12 | C |
| ATOM | 1875 | O | PRO | A | 408 | −6.389 | 30.056 | 25.688 | 1.00 | 29.12 | O |
| ATOM | 1876 | CB | PRO | A | 408 | −8.835 | 31.967 | 26.054 | 1.00 | 25.96 | C |
| ATOM | 1877 | CG | PRO | A | 408 | −8.824 | 33.331 | 26.690 | 1.00 | 25.60 | C |
| ATOM | 1878 | CD | PRO | A | 408 | −7.376 | 33.449 | 27.142 | 1.00 | 26.30 | C |
| ATOM | 1879 | N | GLU | A | 409 | −7.941 | 28.731 | 26.646 | 1.00 | 31.66 | N |
| ATOM | 1880 | CA | GLU | A | 409 | −7.441 | 27.479 | 26.078 | 1.00 | 34.64 | C |
| ATOM | 1881 | C | GLU | A | 409 | −6.104 | 27.014 | 26.661 | 1.00 | 34.56 | C |
| ATOM | 1882 | O | GLU | A | 409 | −5.480 | 26.100 | 26.122 | 1.00 | 34.24 | O |
| ATOM | 1883 | CB | GLU | A | 409 | −7.293 | 27.606 | 24.555 | 1.00 | 37.88 | C |
| ATOM | 1884 | CG | GLU | A | 409 | −8.511 | 28.167 | 23.823 | 1.00 | 43.65 | C |
| ATOM | 1885 | CD | GLU | A | 409 | −9.724 | 27.259 | 23.887 | 1.00 | 46.69 | C |
| ATOM | 1886 | OE1 | GLU | A | 409 | −10.252 | 27.039 | 24.998 | 1.00 | 49.95 | O |
| ATOM | 1887 | OE2 | GLU | A | 409 | −10.153 | 26.766 | 22.821 | 1.00 | 49.48 | O |
| ATOM | 1888 | N | CYS | A | 410 | −5.671 | 27.628 | 27.759 | 1.00 | 33.81 | N |
| ATOM | 1889 | CA | CYS | A | 410 | −4.399 | 27.267 | 28.382 | 1.00 | 35.08 | C |
| ATOM | 1890 | C | CYS | A | 410 | −4.396 | 25.871 | 29.002 | 1.00 | 34.78 | C |
| ATOM | 1891 | O | CYS | A | 410 | −3.390 | 25.164 | 28.943 | 1.00 | 34.24 | O |
| ATOM | 1892 | CB | CYS | A | 410 | −4.027 | 28.299 | 29.455 | 1.00 | 36.13 | C |
| ATOM | 1893 | SG | CYS | A | 410 | −2.433 | 28.006 | 30.271 | 1.00 | 41.53 | S |
| ATOM | 1894 | N | SER | A | 411 | −5.518 | 25.472 | 29.593 | 1.00 | 34.62 | N |
| ATOM | 1895 | CA | SER | A | 411 | −5.611 | 24.163 | 30.235 | 1.00 | 35.60 | C |
| ATOM | 1896 | C | SER | A | 411 | −5.215 | 23.008 | 29.319 | 1.00 | 35.51 | C |
| ATOM | 1897 | O | SER | A | 411 | −4.602 | 22.040 | 29.770 | 1.00 | 35.62 | O |
| ATOM | 1898 | CB | SER | A | 411 | −7.031 | 23.928 | 30.763 | 1.00 | 36.58 | C |
| ATOM | 1899 | OG | SER | A | 411 | −7.959 | 23.837 | 29.697 | 1.00 | 38.87 | O |
| ATOM | 1900 | N | MET | A | 412 | −5.561 | 23.108 | 28.038 | 1.00 | 35.70 | N |
| ATOM | 1901 | CA | MET | A | 412 | −5.244 | 22.053 | 27.079 | 1.00 | 36.25 | C |
| ATOM | 1902 | C | MET | A | 412 | −3.744 | 21.912 | 26.846 | 1.00 | 34.53 | C |
| ATOM | 1903 | O | MET | A | 412 | −3.273 | 20.867 | 26.393 | 1.00 | 34.48 | O |
| ATOM | 1904 | CB | MET | A | 412 | −5.936 | 22.324 | 25.741 | 1.00 | 40.58 | C |
| ATOM | 1905 | CG | MET | A | 412 | −7.433 | 22.563 | 25.850 | 1.00 | 45.64 | C |
| ATOM | 1906 | SD | MET | A | 412 | −8.214 | 22.729 | 24.232 | 1.00 | 52.62 | S |
| ATOM | 1907 | CE | MET | A | 412 | −7.402 | 24.204 | 23.610 | 1.00 | 50.53 | C |
| ATOM | 1908 | N | LYS | A | 413 | −2.996 | 22.965 | 27.150 | 1.00 | 31.53 | N |
| ATOM | 1909 | CA | LYS | A | 413 | −1.551 | 22.944 | 26.960 | 1.00 | 30.85 | C |
| ATOM | 1910 | C | LYS | A | 413 | −0.831 | 22.407 | 28.192 | 1.00 | 30.52 | C |
| ATOM | 1911 | O | LYS | A | 413 | 0.386 | 22.236 | 28.187 | 1.00 | 30.68 | O |
| ATOM | 1912 | CB | LYS | A | 413 | −1.042 | 24.350 | 26.632 | 1.00 | 31.05 | C |
| ATOM | 1913 | CG | LYS | A | 413 | −1.557 | 24.897 | 25.307 | 1.00 | 32.36 | C |
| ATOM | 1914 | CD | LYS | A | 413 | −1.030 | 26.296 | 25.035 | 1.00 | 32.77 | C |
| ATOM | 1915 | CB | LYS | A | 413 | −1.521 | 26.812 | 23.689 | 1.00 | 34.50 | C |
| ATOM | 1916 | NZ | LYS | A | 413 | −3.014 | 26.878 | 23.622 | 1.00 | 36.17 | N |
| ATOM | 1917 | N | LEU | A | 414 | −1.590 | 22.142 | 29.248 | 1.00 | 30.09 | N |
| ATOM | 1918 | CA | LEU | A | 414 | −1.014 | 21.620 | 30.484 | 1.00 | 28.96 | C |
| ATOM | 1919 | C | LEU | A | 414 | −1.393 | 20.147 | 30.610 | 1.00 | 28.33 | C |
| ATOM | 1920 | O | LEU | A | 414 | −1.654 | 19.489 | 29.604 | 1.00 | 29.10 | O |
| ATOM | 1921 | CB | LEU | A | 414 | −1.544 | 22.427 | 31.676 | 1.00 | 28.64 | C |
| ATOM | 1922 | CG | LEU | A | 414 | −1.270 | 23.934 | 31.581 | 1.00 | 30.41 | C |
| ATOM | 1923 | CD1 | LEU | A | 414 | −1.967 | 24.676 | 32.711 | 1.00 | 31.19 | C |
| ATOM | 1924 | CD2 | LEU | A | 414 | 0.226 | 24.179 | 31.624 | 1.00 | 30.79 | C |
| ATOM | 1925 | N | THR | A | 415 | −1.401 | 19.624 | 31.833 | 1.00 | 27.70 | N |
| ATOM | 1926 | CA | THR | A | 415 | −1.779 | 18.232 | 32.071 | 1.00 | 26.69 | C |
| ATOM | 1927 | C | THR | A | 415 | −2.620 | 18.195 | 33.338 | 1.00 | 26.91 | C |
| ATOM | 1928 | O | THR | A | 415 | −2.548 | 19.104 | 34.157 | 1.00 | 26.39 | O |
| ATOM | 1929 | CB | THR | A | 415 | −0.556 | 17.310 | 32.307 | 1.00 | 26.61 | C |
| ATOM | 1930 | OG1 | THR | A | 415 | −0.006 | 17.570 | 33.607 | 1.00 | 25.35 | O |
| ATOM | 1931 | CG2 | THR | A | 415 | 0.509 | 17.546 | 31.247 | 1.00 | 26.48 | C |
| ATOM | 1932 | N | PRO | A | 416 | −3.432 | 17.142 | 33.516 | 1.00 | 27.60 | N |
| ATOM | 1933 | CA | PRO | A | 416 | −4.269 | 17.037 | 34.717 | 1.00 | 27.16 | C |
| ATOM | 1934 | C | PRO | A | 416 | −3.477 | 17.169 | 36.026 | 1.00 | 27.48 | C |
| ATOM | 1935 | O | PRO | A | 416 | −3.930 | 17.813 | 36.975 | 1.00 | 26.90 | O |
| ATOM | 1936 | CB | PRO | A | 416 | −4.908 | 15.661 | 34.564 | 1.00 | 29.00 | C |
| ATOM | 1937 | CG | PRO | A | 416 | −5.083 | 15.555 | 33.072 | 1.00 | 28.36 | C |
| ATOM | 1938 | CD | PRO | A | 416 | −3.752 | 16.071 | 32.553 | 1.00 | 28.22 | C |
| ATOM | 1939 | N | LEU | A | 417 | −2.294 | 16.560 | 36.072 | 1.00 | 25.90 | N |
| ATOM | 1940 | CA | LEU | A | 417 | −1.460 | 16.610 | 37.271 | 1.00 | 25.39 | C |
| ATOM | 1941 | C | LEU | A | 417 | −0.961 | 18.031 | 37.545 | 1.00 | 24.67 | C |
| ATOM | 1942 | O | LEU | A | 417 | −0.983 | 18.502 | 38.685 | 1.00 | 24.55 | O |
| ATOM | 1943 | CB | LEU | A | 417 | −0.279 | 15.643 | 37.124 | 1.00 | 25.12 | C |
| ATOM | 1944 | CG | LEU | A | 417 | 0.722 | 15.507 | 38.273 | 1.00 | 25.26 | C |
| ATOM | 1945 | CD1 | LEU | A | 417 | 0.021 | 15.098 | 39.564 | 1.00 | 24.40 | C |
| ATOM | 1946 | CD2 | LEU | A | 417 | 1.766 | 14.470 | 37.882 | 1.00 | 25.23 | C |
| ATOM | 1947 | N | VAL | A | 418 | −0.506 | 18.711 | 36.500 | 1.00 | 24.66 | N |
| ATOM | 1948 | CA | VAL | A | 418 | −0.027 | 20.080 | 36.640 | 1.00 | 25.57 | C |
| ATOM | 1949 | C | VAL | A | 418 | −1.176 | 20.971 | 37.111 | 1.00 | 26.33 | C |
| ATOM | 1950 | O | VAL | A | 418 | −1.001 | 21.814 | 37.991 | 1.00 | 27.09 | O |
| ATOM | 1951 | CB | VAL | A | 418 | 0.531 | 20.599 | 35.297 | 1.00 | 25.14 | C |
| ATOM | 1952 | CG1 | VAL | A | 418 | 0.723 | 22.112 | 35.338 | 1.00 | 26.89 | C |
| ATOM | 1953 | CG2 | VAL | A | 418 | 1.861 | 19.912 | 35.009 | 1.00 | 25.97 | C |
| ATOM | 1954 | N | LEU | A | 419 | −2.354 | 20.769 | 36.530 | 1.00 | 26.33 | N |
| ATOM | 1955 | CA | LEU | A | 419 | −3.526 | 21.556 | 36.902 | 1.00 | 27.78 | C |
| ATOM | 1956 | C | LEU | A | 419 | −3.861 | 21.399 | 38.382 | 1.00 | 29.03 | C |
| ATOM | 1957 | O | LEU | A | 419 | −4.206 | 22.370 | 39.052 | 1.00 | 30.30 | O |
| ATOM | 1958 | CB | LEU | A | 419 | −4.733 | 21.143 | 36.051 | 1.00 | 28.60 | C |
| ATOM | 1959 | CG | LEU | A | 419 | −4.696 | 21.585 | 34.586 | 1.00 | 30.69 | C |
| ATOM | 1960 | CD1 | LEU | A | 419 | −5.871 | 20.975 | 33.828 | 1.00 | 30.94 | C |
| ATOM | 1961 | CD2 | LEU | A | 419 | −4.743 | 23.105 | 34.515 | 1.00 | 31.11 | C |
| ATOM | 1962 | N | GLU | A | 420 | −3.738 | 20.184 | 38.904 | 1.00 | 29.76 | N |
| ATOM | 1963 | CA | GLU | A | 420 | −4.056 | 19.962 | 40.307 | 1.00 | 31.06 | C |
| ATOM | 1964 | C | GLU | A | 420 | −3.010 | 20.514 | 41.268 | 1.00 | 30.59 | C |
| ATOM | 1965 | O | GLU | A | 420 | −3.344 | 21.184 | 42.245 | 1.00 | 30.30 | O |
| ATOM | 1966 | CB | GLU | A | 420 | −4.237 | 18.478 | 40.605 | 1.00 | 32.62 | C |
| ATOM | 1967 | CG | GLU | A | 420 | −4.697 | 18.251 | 42.037 | 1.00 | 36.69 | C |
| ATOM | 1968 | CD | GLU | A | 420 | −4.267 | 16.919 | 42.598 | 1.00 | 38.47 | C |
| ATOM | 1969 | OE1 | GLU | A | 420 | −4.631 | 16.624 | 43.756 | 1.00 | 40.46 | O |
| ATOM | 1970 | OE2 | GLU | A | 420 | −3.561 | 16.171 | 41.891 | 1.00 | 41.56 | O |
| ATOM | 1971 | N | VAL | A | 421 | −1.744 | 20.223 | 40.992 | 1.00 | 31.25 | N |
| ATOM | 1972 | CA | VAL | A | 421 | −0.663 | 20.675 | 41.855 | 1.00 | 32.00 | C |
| ATOM | 1973 | C | VAL | A | 421 | −0.544 | 22.191 | 41.960 | 1.00 | 32.63 | C |
| ATOM | 1974 | O | VAL | A | 421 | −0.355 | 22.724 | 43.051 | 1.00 | 32.82 | O |
| ATOM | 1975 | CB | VAL | A | 421 | 0.694 | 20.082 | 41.395 | 1.00 | 31.60 | C |
| ATOM | 1976 | CG1 | VAL | A | 421 | 1.843 | 20.676 | 42.208 | 1.00 | 31.31 | C |
| ATOM | 1977 | CG2 | VAL | A | 421 | 0.667 | 18.567 | 41.556 | 1.00 | 31.20 | C |
| ATOM | 1978 | N | PHE | A | 422 | −0.670 | 22.890 | 40.839 | 1.00 | 33.47 | N |
| ATOM | 1979 | CA | PHE | A | 422 | −0.541 | 24.342 | 40.857 | 1.00 | 34.95 | C |
| ATOM | 1980 | C | PHE | A | 422 | −1.866 | 25.089 | 40.872 | 1.00 | 35.99 | C |
| ATOM | 1981 | O | PHE | A | 422 | −1.907 | 26.284 | 41.159 | 1.00 | 36.37 | O |
| ATOM | 1982 | CB | PHE | A | 422 | 0.310 | 24.794 | 39.670 | 1.00 | 34.60 | C |
| ATOM | 1983 | CG | PHE | A | 422 | 1.679 | 24.182 | 39.656 | 1.00 | 34.76 | C |
| ATOM | 1984 | CD1 | PHE | A | 422 | 2.093 | 23.389 | 38.592 | 1.00 | 34.88 | C |
| ATOM | 1985 | CD2 | PHE | A | 422 | 2.545 | 24.369 | 40.728 | 1.00 | 35.16 | C |
| ATOM | 1986 | CE1 | PHE | A | 422 | 3.348 | 22.790 | 38.597 | 1.00 | 34.92 | C |
| ATOM | 1987 | CE2 | PHE | A | 422 | 3.801 | 23.774 | 40.743 | 1.00 | 34.70 | C |
| ATOM | 1988 | CZ | PHE | A | 422 | 4.202 | 22.982 | 39.674 | 1.00 | 34.73 | C |
| ATOM | 1989 | N | GLY | A | 423 | −2.946 | 24.378 | 40.570 | 1.00 | 37.39 | N |
| ATOM | 1990 | CA | GLY | A | 423 | −4.261 | 24.993 | 40.564 | 1.00 | 38.89 | C |
| ATOM | 1991 | C | GLY | A | 423 | −4.914 | 24.907 | 41.930 | 1.00 | 39.70 | C |
| ATOM | 1992 | O | GLY | A | 423 | −5.857 | 24.099 | 42.083 | 1.00 | 40.52 | O |
| TER | 1993 | | GLY | A | 423 | | | | | | |
| HETATM | 1994 | O2 | VDX | | 425 | 17.029 | 18.071 | 34.819 | 1.00 | 21.73 | O |
| HETATM | 1995 | O3 | VDX | | 425 | 4.489 | 26.946 | 35.054 | 1.00 | 24.67 | O |
| HETATM | 1996 | C1 | VDX | | 425 | 14.139 | 17.953 | 35.755 | 1.00 | 20.80 | C |
| HETATM | 1997 | C2 | VDX | | 425 | 14.879 | 16.893 | 34.895 | 1.00 | 21.02 | C |
| HETATM | 1998 | C3 | VDX | | 425 | 15.992 | 17.534 | 33.962 | 1.00 | 21.41 | C |
| HETATM | 1999 | C4 | VDX | | 425 | 15.368 | 18.672 | 33.049 | 1.00 | 21.29 | C |
| HETATM | 2000 | C5 | VDX | | 425 | 14.622 | 19.724 | 33.864 | 1.00 | 21.00 | C |
| HETATM | 2001 | C6 | VDX | | 425 | 14.797 | 21.120 | 33.792 | 1.00 | 20.95 | C |
| HETATM | 2002 | C7 | VDX | | 425 | 14.174 | 22.286 | 34.514 | 1.00 | 21.23 | C |
| HETATM | 2003 | C8 | VDX | | 425 | 13.966 | 23.488 | 34.042 | 1.00 | 21.54 | C |
| HETATM | 2004 | C9 | VDX | | 425 | 14.354 | 23.927 | 32.544 | 1.00 | 21.77 | C |
| HETATM | 2005 | C10 | VDX | | 425 | 13.602 | 19.075 | 34.828 | 1.00 | 20.74 | C |
| HETATM | 2006 | C11 | VDX | | 425 | 13.088 | 24.490 | 31.671 | 1.00 | 21.66 | C |
| HETATM | 2007 | C12 | VDX | | 425 | 12.147 | 25.443 | 32.564 | 1.00 | 22.04 | C |
| HETATM | 2008 | C13 | VDX | | 425 | 11.753 | 24.897 | 34.070 | 1.00 | 22.01 | C |
| HETATM | 2009 | C14 | VDX | | 425 | 13.148 | 24.538 | 34.777 | 1.00 | 21.80 | C |
| HETATM | 2010 | C15 | VDX | | 425 | 12.661 | 24.266 | 36.350 | 1.00 | 22.22 | C |
| HETATM | 2011 | C16 | VDX | | 425 | 11.429 | 25.231 | 36.497 | 1.00 | 22.39 | C |
| HETATM | 2012 | C17 | VDX | | 425 | 11.276 | 25.934 | 35.106 | 1.00 | 22.31 | C |
| HETATM | 2013 | C18 | VDX | | 425 | 10.769 | 23.570 | 33.779 | 1.00 | 21.50 | C |
| HETATM | 2014 | C19 | VDX | | 425 | 12.291 | 19.455 | 34.852 | 1.00 | 20.77 | C |
| HETATM | 2015 | C20 | VDX | | 425 | 9.849 | 26.546 | 34.726 | 1.00 | 22.90 | C |
| HETATM | 2016 | C21 | VDX | | 425 | 9.804 | 27.956 | 35.482 | 1.00 | 23.65 | C |
| HETATM | 2017 | C22 | VDX | | 425 | 8.575 | 25.824 | 35.268 | 1.00 | 23.16 | C |
| HETATM | 2018 | C23 | VDX | | 425 | 7.331 | 26.060 | 34.405 | 1.00 | 23.73 | C |
| HETATM | 2019 | C24 | VDX | | 425 | 6.152 | 25.266 | 34.672 | 1.00 | 24.36 | C |
| HETATM | 2020 | C25 | VDX | | 425 | 4.775 | 25.776 | 34.336 | 1.00 | 24.75 | C |
| HETATM | 2021 | C26 | VDX | | 425 | 4.701 | 26.010 | 32.842 | 1.00 | 25.41 | C |
| HETATM | 2022 | C27 | VDX | | 425 | 3.668 | 24.730 | 34.723 | 1.00 | 25.39 | C |
| HETATM | 2023 | O1 | VDX | | 425 | 13.119 | 17.359 | 36.620 | 1.00 | 20.68 | O |
| HETATM | 2024 | O | HOH | | 500 | 14.347 | 10.333 | 30.796 | 1.00 | 24.33 | O |
| HETATM | 2025 | O | HOH | | 501 | 13.828 | 12.782 | 35.922 | 1.00 | 21.46 | O |
| HETATM | 2026 | O | HOH | | 502 | 13.846 | 14.468 | 42.856 | 1.00 | 24.78 | O |
| HETATM | 2027 | O | HOH | | 503 | 19.132 | 15.890 | 40.266 | 1.00 | 21.27 | O |
| HETATM | 2028 | O | HOH | | 504 | 15.013 | 12.029 | 41.977 | 1.00 | 22.69 | O |
| HETATM | 2029 | O | HOH | | 505 | 13.766 | 10.118 | 35.125 | 1.00 | 20.29 | O |
| HETATM | 2030 | O | HOH | | 506 | 16.290 | 13.157 | 34.345 | 1.00 | 30.57 | O |
| HETATM | 2031 | O | HOH | | 507 | 5.938 | 22.747 | 23.179 | 1.00 | 24.25 | O |
| HETATM | 2032 | O | HOH | | 508 | 13.771 | 7.592 | 35.963 | 1.00 | 28.23 | O |
| HETATM | 2033 | O | HOH | | 509 | 12.348 | 25.386 | 50.763 | 1.00 | 30.93 | O |
| HETATM | 2034 | O | HOH | | 510 | 28.498 | 23.703 | 34.824 | 1.00 | 37.09 | O |
| HETATM | 2035 | O | HOH | | 511 | 26.394 | 10.521 | 64.086 | 1.00 | 30.68 | O |
| HETATM | 2036 | O | HOH | | 512 | 20.573 | 9.150 | 38.613 | 1.00 | 30.36 | O |
| HETATM | 2037 | O | HOH | | 513 | 19.724 | 30.629 | 29.203 | 1.00 | 35.40 | O |
| HETATM | 2038 | O | HOH | | 514 | 4.372 | 27.504 | 42.595 | 1.00 | 31.46 | O |
| HETATM | 2039 | O | HOH | | 515 | 2.808 | 13.423 | 33.286 | 1.00 | 30.93 | O |
| HETATM | 2040 | O | HOH | | 516 | 23.698 | 20.154 | 43.135 | 1.00 | 37.92 | O |
| HETATM | 2041 | O | HOH | | 517 | 11.325 | 5.901 | 37.588 | 1.00 | 30.12 | O |
| HETATM | 2042 | O | HOH | | 518 | 0.885 | 13.049 | 59.537 | 1.00 | 39.32 | O |
| HETATM | 2043 | O | HOH | | 519 | 20.338 | 11.515 | 62.065 | 1.00 | 36.13 | O |
| HETATM | 2044 | O | HOH | | 520 | 8.913 | 6.134 | 53.451 | 1.00 | 44.37 | O |
| HETATM | 2045 | O | HOH | | 521 | 4.924 | 23.321 | 44.129 | 1.00 | 33.51 | O |
| HETATM | 2046 | O | HOH | | 522 | 16.547 | 6.409 | 36.375 | 1.00 | 32.70 | O |
| HETATM | 2047 | O | HOH | | 523 | 8.896 | 35.918 | 45.789 | 1.00 | 45.73 | O |
| HETATM | 2048 | O | HOH | | 524 | 26.192 | 21.542 | 43.420 | 1.00 | 28.56 | O |
| HETATM | 2049 | O | HOH | | 525 | −5.345 | 32.214 | 23.915 | 1.00 | 35.31 | O |
| HETATM | 2050 | O | HOH | | 526 | 9.488 | 15.901 | 22.976 | 1.00 | 29.33 | O |
| HETATM | 2051 | O | HOH | | 527 | 5.345 | 31.465 | 22.796 | 1.00 | 31.37 | O |
| HETATM | 2052 | O | HOH | | 528 | 6.982 | 20.227 | 51.589 | 1.00 | 32.20 | O |
| HETATM | 2053 | O | HOH | | 529 | 4.642 | 13.886 | 30.953 | 1.00 | 31.71 | O |
| HETATM | 2054 | O | HOH | | 530 | −3.764 | 29.115 | 25.550 | 1.00 | 37.63 | O |
| HETATM | 2055 | O | HOH | | 531 | 31.831 | 9.097 | 66.550 | 1.00 | 36.20 | O |
| HETATM | 2056 | O | HOH | | 532 | 10.178 | 6.595 | 32.965 | 1.00 | 30.94 | O |
| HETATM | 2057 | O | HOH | | 533 | −1.561 | 14.197 | 34.245 | 1.00 | 33.20 | O |
| HETATM | 2058 | O | HOH | | 534 | 0.476 | 12.154 | 62.160 | 1.00 | 39.93 | O |
| HETATM | 2059 | O | HOH | | 535 | 25.970 | 5.142 | 53.011 | 1.00 | 47.31 | O |
| HETATM | 2060 | O | HOH | | 536 | 8.695 | 5.045 | 44.801 | 1.00 | 38.39 | O |
| HETATM | 2061 | O | HOH | | 537 | 22.396 | 11.047 | 39.112 | 1.00 | 40.45 | O |
| HETATM | 2062 | O | HOH | | 538 | 13.975 | 29.983 | 22.553 | 1.00 | 36.21 | O |
| HETATM | 2063 | O | HOH | | 539 | −6.673 | 18.195 | 37.122 | 1.00 | 36.41 | O |
| HETATM | 2064 | O | HOH | | 540 | 15.926 | 27.813 | 55.197 | 1.00 | 43.43 | O |
| HETATM | 2065 | O | HOH | | 541 | 21.922 | 29.786 | 26.625 | 1.00 | 39.42 | O |
| HETATM | 2066 | O | HOH | | 542 | 29.079 | 22.924 | 57.335 | 1.00 | 43.49 | O |
| HETATM | 2067 | O | HOH | | 543 | −8.883 | 26.986 | 29.744 | 1.00 | 47.42 | O |
| HETATM | 2068 | O | HOH | | 544 | −2.789 | 31.232 | 23.837 | 1.00 | 38.14 | O |
| HETATM | 2069 | O | HOH | | 545 | 15.578 | 33.329 | 45.128 | 1.00 | 39.44 | O |
| HETATM | 2070 | O | HOH | | 546 | 20.810 | 2.660 | 42.920 | 1.00 | 51.44 | O |
| HETATM | 2071 | O | HOH | | 547 | 27.448 | 25.982 | 58.310 | 1.00 | 43.04 | O |
| HETATM | 2072 | O | HOH | | 548 | 21.987 | 8.152 | 64.287 | 1.00 | 43.15 | O |
| HETATM | 2073 | O | HOH | | 549 | 14.435 | 13.091 | 64.840 | 1.00 | 35.87 | O |
| HETATM | 2074 | O | HOH | | 550 | 1.276 | 25.772 | 21.944 | 1.00 | 40.66 | O |
| HETATM | 2075 | O | HOH | | 551 | 14.102 | 6.513 | 31.763 | 1.00 | 43.70 | O |
| HETATM | 2076 | O | HOH | | 552 | 11.990 | 24.017 | 53.147 | 1.00 | 45.62 | O |
| HETATM | 2077 | O | HOH | | 553 | 3.481 | 24.236 | 20.666 | 1.00 | 35.69 | O |
| HETATM | 2078 | O | HOH | | 554 | 24.054 | 13.110 | 35.770 | 1.00 | 37.92 | O |
| HETATM | 2079 | O | HOH | | 556 | 6.857 | 37.182 | 44.351 | 1.00 | 49.60 | O |
| HETATM | 2080 | O | HOH | | 557 | −8.644 | 30.901 | 30.925 | 1.00 | 41.21 | O |
| HETATM | 2081 | O | HOH | | 558 | 17.767 | 33.571 | 43.159 | 1.00 | 37.66 | O |
| HETATM | 2082 | O | HOH | | 559 | 16.954 | 26.537 | 23.238 | 1.00 | 51.77 | O |
| HETATM | 2083 | O | HOH | | 560 | 27.386 | 20.638 | 40.959 | 1.00 | 37.25 | O |
| HETATM | 2084 | O | HOH | | 561 | 31.418 | 10.182 | 50.496 | 1.00 | 47.27 | O |
| HETATM | 2085 | O | HOH | | 562 | 4.082 | 21.082 | 20.610 | 1.00 | 37.94 | O |
| HETATM | 2086 | O | HOH | | 563 | 14.064 | 10.706 | 58.224 | 1.00 | 42.75 | O |
| HETATM | 2087 | O | HOH | | 564 | 23.415 | 29.835 | 49.803 | 1.00 | 45.77 | O |
| HETATM | 2088 | O | HOH | | 565 | 14.533 | 11.393 | 24.395 | 1.00 | 36.60 | O |
| HETATM | 2089 | O | HOH | | 566 | −0.868 | 36.798 | 40.025 | 1.00 | 52.17 | O |
| HETATM | 2090 | O | HOH | | 567 | 2.865 | 34.386 | 33.570 | 1.00 | 42.56 | O |
| HETATM | 2091 | O | HOH | | 568 | −4.893 | 19.288 | 30.751 | 1.00 | 44.30 | O |
| HETATM | 2092 | O | HOH | | 569 | 30.643 | 14.674 | 61.949 | 1.00 | 43.28 | O |
| HETATM | 2093 | O | HOH | | 570 | 22.702 | 3.372 | 47.417 | 1.00 | 36.93 | O |
| HETATM | 2094 | O | HOH | | 571 | 13.379 | 35.172 | 44.109 | 1.00 | 47.38 | O |
| HETATM | 2095 | O | HOH | | 572 | −1.138 | 20.698 | 22.966 | 1.00 | 53.61 | O |
| HETATM | 2096 | O | HOH | | 573 | 25.589 | 19.849 | 33.401 | 1.00 | 52.13 | O |
| HETATM | 2097 | O | HOH | | 574 | 23.893 | 13.360 | 32.579 | 1.00 | 45.26 | O |
| HETATM | 2098 | O | HOH | | 575 | −7.367 | 18.485 | 31.944 | 1.00 | 48.23 | O |
| HETATM | 2099 | O | HOH | | 576 | 2.430 | 19.200 | 65.790 | 1.00 | 45.13 | O |
| HETATM | 2100 | O | HOH | | 577 | 20.048 | 32.028 | 44.907 | 1.00 | 46.82 | O |
| HETATM | 2101 | O | HOH | | 578 | 20.286 | 6.713 | 37.519 | 1.00 | 43.08 | O |
| HETATM | 2102 | O | HOH | | 579 | 25.879 | 5.448 | 50.403 | 1.00 | 48.82 | O |
| HETATM | 2103 | O | HOH | | 580 | 24.905 | 19.763 | 39.659 | 1.00 | 45.39 | O |
| HETATM | 2104 | O | HOH | | 581 | 2.341 | 14.233 | 26.082 | 1.00 | 50.76 | O |
| HETATM | 2105 | O | HOH | | 582 | 15.248 | 20.000 | 60.506 | 1.00 | 44.08 | O |
| HETATM | 2106 | O | HOH | | 583 | 22.695 | 7.038 | 37.715 | 1.00 | 46.55 | O |
| HETATM | 2107 | O | HOH | | 584 | 11.915 | 16.625 | 66.479 | 1.00 | 52.58 | O |
| HETATM | 2108 | O | HOH | | 585 | 20.145 | 35.730 | 35.936 | 1.00 | 46.90 | O |
| HETATM | 2109 | O | HOH | | 586 | 10.735 | 24.933 | 16.684 | 1.00 | 46.64 | O |
| HETATM | 2110 | O | HOH | | 587 | 1.182 | 9.495 | 61.830 | 1.00 | 55.88 | O |
| HETATM | 2111 | O | HOH | | 588 | −3.993 | 16.527 | 51.745 | 1.00 | 43.33 | O |
| HETATM | 2112 | O | HOH | | 589 | 21.842 | 29.919 | 56.624 | 1.00 | 42.17 | O |
| HETATM | 2113 | O | HOH | | 590 | 3.602 | 25.520 | 44.494 | 1.00 | 50.24 | O |
| HETATM | 2114 | O | HOH | | 591 | 1.198 | 23.984 | 44.777 | 1.00 | 43.76 | O |
| HETATM | 2115 | O | HOH | | 592 | 13.208 | 27.713 | 54.123 | 1.00 | 59.17 | O |
| HETATM | 2116 | O | HOH | | 593 | 27.958 | 7.530 | 50.434 | 1.00 | 53.55 | O |
| HETATM | 2117 | O | HOH | | 594 | 22.594 | 3.510 | 64.140 | 1.00 | 45.66 | O |
| HETATM | 2118 | O | HOH | | 595 | 30.412 | 22.979 | 36.623 | 1.00 | 71.37 | O |
| HETATM | 2119 | O | HOH | | 596 | 10.560 | 15.906 | 20.574 | 1.00 | 50.32 | O |
| HETATM | 2120 | O | HOH | | 597 | 26.021 | 3.241 | 64.667 | 1.00 | 49.85 | O |
| HETATM | 2121 | O | HOH | | 598 | 19.853 | 9.062 | 62.967 | 1.00 | 56.45 | O |
| HETATM | 2122 | O | HOH | | 599 | 12.462 | 3.992 | 52.363 | 1.00 | 42.46 | O |
| HETATM | 2123 | O | HOH | | 600 | 6.152 | 35.657 | 28.721 | 1.00 | 46.87 | O |
| HETATM | 2124 | O | HOH | | 601 | 7.626 | 29.983 | 53.085 | 1.00 | 51.73 | O |
| HETATM | 2125 | O | HOH | | 602 | 11.547 | 23.591 | 57.064 | 1.00 | 51.07 | O |
| HETATM | 2126 | O | HOH | | 603 | 24.407 | 19.393 | 31.035 | 1.00 | 53.85 | O |
| HETATM | 2127 | O | HOH | | 604 | 12.538 | 23.006 | 18.706 | 1.00 | 50.11 | O |
| HETATM | 2128 | O | HOH | | 605 | 1.839 | 16.469 | 66.997 | 1.00 | 49.40 | O |
| HETATM | 2129 | O | HOH | | 606 | 1.378 | 19.964 | 21.070 | 1.00 | 48.97 | O |
| HETATM | 2130 | O | HOH | | 607 | 5.895 | 26.935 | 51.419 | 1.00 | 53.95 | O |
| HETATM | 2131 | O | HOH | | 608 | 13.122 | 33.698 | 19.464 | 1.00 | 52.90 | O |
| HETATM | 2132 | O | HOH | | 609 | 27.040 | 8.636 | 44.102 | 1.00 | 44.22 | O |
| HETATM | 2133 | O | HOH | | 610 | 18.833 | 30.775 | 55.879 | 1.00 | 54.75 | O |
| HETATM | 2134 | O | HOH | | 611 | 34.509 | 17.720 | 47.771 | 1.00 | 42.84 | O |
| HETATM | 2135 | O | HOH | | 612 | 18.356 | 32.644 | 25.579 | 1.00 | 42.52 | O |
| HETATM | 2136 | O | HOH | | 613 | −2.259 | 16.235 | 28.804 | 1.00 | 56.71 | O |
| HETATM | 2137 | O | HOH | | 614 | 16.400 | 38.404 | 21.700 | 1.00 | 46.19 | O |
| HETATM | 2138 | O | HOH | | 615 | 9.340 | 39.540 | 19.060 | 1.00 | 51.44 | O |
| HETATM | 2139 | O | HOH | | 616 | 20.026 | 35.074 | 32.855 | 1.00 | 47.06 | O |
| HETATM | 2140 | O | HOH | | 617 | 31.604 | 8.486 | 59.428 | 1.00 | 47.99 | O |
| HETATM | 2141 | O | HOH | | 618 | 26.228 | 8.975 | 40.708 | 1.00 | 47.20 | O |
| HETATM | 2142 | O | HOH | | 619 | 0.460 | 15.378 | 28.064 | 1.00 | 50.21 | O |
| HETATM | 2143 | O | HOH | | 620 | 15.771 | 3.385 | 48.139 | 1.00 | 38.09 | O |
| HETATM | 2144 | O | HOH | | 621 | 25.135 | 17.914 | 42.644 | 1.00 | 60.05 | O |
| HETATM | 2145 | O | HOH | | 622 | −2.286 | 29.197 | 21.618 | 1.00 | 53.99 | O |
| HETATM | 2146 | O | HOH | | 623 | 32.865 | 18.926 | 45.658 | 1.00 | 48.11 | O |
| HETATM | 2147 | O | HOH | | 624 | 17.116 | 13.333 | 25.240 | 1.00 | 52.60 | O |
| HETATM | 2148 | O | HOH | | 625 | −2.809 | 17.978 | 56.255 | 1.00 | 53.36 | O |
| HETATM | 2149 | O | HOH | | 626 | −3.647 | 7.885 | 56.347 | 1.00 | 63.91 | O |
| HETATM | 2150 | O | HOH | | 627 | 17.746 | 24.596 | 21.608 | 1.00 | 59.81 | O |
| HETATM | 2151 | O | HOH | | 628 | 28.368 | 5.841 | 47.861 | 1.00 | 66.08 | O |
| HETATM | 2152 | O | HOH | | 629 | 13.641 | 11.618 | 66.858 | 1.00 | 52.02 | O |
| HETATM | 2153 | O | HOH | | 630 | 8.052 | 20.893 | 16.742 | 1.00 | 53.91 | O |
| HETATM | 2154 | O | HOH | | 631 | 8.914 | 38.015 | 27.578 | 1.00 | 56.47 | O |
| HETATM | 2155 | O | HOH | | 632 | 9.081 | 13.482 | 19.627 | 1.00 | 57.14 | O |
| HETATM | 2156 | O | HOH | | 633 | −4.343 | 24.969 | 37.694 | 1.00 | 51.08 | O |
| HETATM | 2157 | O | HOH | | 634 | 3.597 | 28.859 | 46.576 | 1.00 | 54.80 | O |
| HETATM | 2158 | O | HOH | | 635 | 27.905 | 21.432 | 28.373 | 1.00 | 59.49 | O |
| HETATM | 2159 | O | HOH | | 636 | −4.252 | 18.337 | 25.491 | 1.00 | 47.50 | O |
| HETATM | 2160 | O | HOH | | 637 | −2.808 | 23.046 | 51.839 | 1.00 | 49.04 | O |
| HETATM | 2161 | O | HOH | | 638 | 2.757 | 25.756 | 18.437 | 1.00 | 49.80 | O |
| HETATM | 2162 | O | HOH | | 639 | 15.470 | 7.390 | 63.803 | 1.00 | 52.42 | O |
| HETATM | 2163 | O | HOH | | 640 | 33.689 | 11.757 | 50.784 | 1.00 | 54.00 | O |
| HETATM | 2164 | O | HOH | | 641 | 6.223 | 13.352 | 20.927 | 1.00 | 49.77 | O |
| HETATM | 2165 | O | HOH | | 642 | 12.267 | 32.764 | 51.605 | 1.00 | 48.76 | O |
| HETATM | 2166 | O | HOH | | 644 | 25.211 | 3.585 | 48.391 | 1.00 | 49.75 | O |
| HETATM | 2167 | O | HOH | | 645 | 0.619 | 24.002 | 51.358 | 1.00 | 49.46 | O |
| HETATM | 2168 | O | HOH | | 646 | 12.270 | 22.627 | 60.617 | 1.00 | 63.88 | O |
| HETATM | 2169 | O | HOH | | 647 | 0.202 | 23.805 | 47.834 | 1.00 | 52.54 | O |
| HETATM | 2170 | O | HOH | | 648 | 15.471 | 8.169 | 23.816 | 1.00 | 54.49 | O |
| HETATM | 2171 | O | HOH | | 649 | 4.098 | 13.117 | 28.105 | 1.00 | 43.97 | O |
| HETATM | 2172 | O | HOH | | 650 | 16.032 | 4.857 | 59.064 | 1.00 | 55.67 | O |
| HETATM | 2173 | O | HOH | | 651 | −5.591 | 11.911 | 55.960 | 1.00 | 63.35 | O |
| HETATM | 2174 | O | HOH | | 652 | 14.373 | 4.083 | 36.218 | 1.00 | 49.18 | O |
| HETATM | 2175 | O | HOH | | 653 | 11.138 | 5.501 | 59.825 | 1.00 | 51.19 | O |
| HETATM | 2176 | O | HOH | | 654 | 26.262 | 1.299 | 50.288 | 1.00 | 61.20 | O |
| HETATM | 2177 | O | HOH | | 655 | 4.067 | 20.751 | 67.111 | 1.00 | 51.75 | O |
| HETATM | 2178 | O | HOH | | 656 | 11.291 | 34.551 | 23.646 | 1.00 | 53.35 | O |
| HETATM | 2179 | O | HOH | | 657 | 2.505 | 33.743 | 45.342 | 1.00 | 58.29 | O |
| HETATM | 2180 | O | HOH | | 658 | 18.881 | −0.886 | 43.452 | 1.00 | 60.82 | O |
| HETATM | 2181 | O | HOH | | 659 | −1.930 | 13.191 | 62.255 | 1.00 | 65.05 | O |
| HETATM | 2182 | O | HOH | | 660 | −3.587 | 12.153 | 34.625 | 1.00 | 51.24 | O |
| HETATM | 2183 | O | HOH | | 661 | −2.064 | 26.008 | 58.110 | 1.00 | 58.94 | O |
| HETATM | 2184 | O | HOH | | 662 | 18.842 | 12.351 | 64.527 | 1.00 | 60.06 | O |
| HETATM | 2185 | O | HOH | | 663 | 30.991 | 26.420 | 51.105 | 1.00 | 54.69 | O |
| HETATM | 2186 | O | HOH | | 664 | 16.115 | 30.354 | 56.207 | 1.00 | 60.96 | O |
| HETATM | 2187 | O | HOH | | 665 | 36.596 | 19.242 | 55.988 | 1.00 | 55.83 | O |

TABLE 3
Atomic Structure Coordinate Data of
Polyalanine Model of Conserved VDR LBD

| | | | | | | | | | | | |
|---|---|---|---|---|---|---|---|---|---|---|---|
| ATOM | 1 | CB | PRO | 103 | −17.052 | −26.771 | 140.477 | 1.00 | 78.63 | A | C |
| ATOM | 2 | CG | PRO | 103 | −16.933 | −28.077 | 141.262 | 1.00 | 78.57 | A | C |
| ATOM | 3 | C | PRO | 103 | −15.322 | −25.595 | 139.088 | 1.00 | 78.42 | A | C |
| ATOM | 4 | O | PRO | 103 | −15.845 | −24.542 | 139.459 | 1.00 | 78.37 | A | O |
| ATOM | 5 | N | PRO | 103 | −14.952 | −27.870 | 140.019 | 1.00 | 78.63 | A | N |
| ATOM | 6 | CD | PRO | 103 | −15.422 | −28.350 | 141.331 | 1.00 | 78.61 | A | C |
| ATOM | 7 | CA | PRO | 103 | −15.952 | −26.943 | 139.436 | 1.00 | 78.57 | A | C |
| ATOM | 8 | N | VAL | 104 | −14.202 | −25.636 | 138.370 | 1.00 | 78.14 | A | N |
| ATOM | 9 | CA | VAL | 104 | −13.489 | −24.422 | 137.982 | 1.00 | 77.74 | A | C |
| ATOM | 10 | CB | VAL | 104 | −12.020 | −24.729 | 137.584 | 1.00 | 77.77 | A | C |
| ATOM | 11 | CG1 | VAL | 104 | −11.298 | −25.415 | 138.733 | 1.00 | 77.66 | A | C |
| ATOM | 12 | CG2 | VAL | 104 | −11.984 | −25.591 | 136.331 | 1.00 | 77.68 | A | C |
| ATOM | 13 | C | VAL | 104 | −14.153 | −23.671 | 136.828 | 1.00 | 77.43 | A | C |
| ATOM | 14 | O | VAL | 104 | −15.023 | −24.202 | 136.133 | 1.00 | 77.67 | A | O |
| ATOM | 15 | N | GLN | 105 | −13.726 | −22.427 | 136.636 | 1.00 | 76.69 | A | N |
| ATOM | 16 | CA | GLN | 105 | −14.254 | −21.567 | 135.582 | 1.00 | 75.70 | A | C |
| ATOM | 17 | CB | GLN | 105 | −13.976 | −20.099 | 135.918 | 1.00 | 76.09 | A | C |
| ATOM | 18 | CG | GLN | 105 | −12.491 | −19.779 | 136.067 | 1.00 | 76.08 | A | C |
| ATOM | 19 | CD | GLN | 105 | −12.210 | −18.291 | 136.099 | 1.00 | 76.03 | A | C |
| ATOM | 20 | OE1 | GLN | 105 | −12.414 | −17.589 | 135.107 | 1.00 | 75.85 | A | O |
| ATOM | 21 | NE2 | GLN | 105 | −11.739 | −17.800 | 137.241 | 1.00 | 75.74 | A | N |
| ATOM | 22 | C | GLN | 105 | −13.637 | −21.877 | 134.223 | 1.00 | 74.59 | A | C |
| ATOM | 23 | O | GLN | 105 | −12.719 | −22.691 | 134.111 | 1.00 | 74.90 | A | O |
| ATOM | 24 | N | LEU | 106 | −14.150 | −21.211 | 133.193 | 1.00 | 72.98 | A | N |
| ATOM | 25 | CA | LEU | 106 | −13.654 | −21.381 | 131.836 | 1.00 | 71.07 | A | C |
| ATOM | 26 | CB | LEU | 106 | −14.603 | −22.279 | 131.032 | 1.00 | 71.27 | A | C |
| ATOM | 27 | CG | LEU | 106 | −14.142 | −22.724 | 129.638 | 1.00 | 71.35 | A | C |
| ATOM | 28 | CD1 | LEU | 106 | −12.802 | −23.437 | 129.733 | 1.00 | 71.22 | A | C |
| ATOM | 29 | CD2 | LEU | 106 | −15.188 | −23.645 | 129.027 | 1.00 | 71.16 | A | C |
| ATOM | 30 | C | LEU | 106 | −13.537 | −20.002 | 131.185 | 1.00 | 69.48 | A | C |
| ATOM | 31 | O | LEU | 106 | −14.517 | −19.447 | 130.693 | 1.00 | 69.41 | A | O |
| ATOM | 32 | N | SER | 107 | −12.326 | −19.456 | 131.211 | 1.00 | 67.67 | A | N |
| ATOM | 33 | CA | SER | 107 | −12.021 | −18.145 | 130.645 | 1.00 | 65.85 | A | C |
| ATOM | 34 | CB | SER | 107 | −10.516 | −18.043 | 130.383 | 1.00 | 65.62 | A | C |
| ATOM | 35 | OG | SER | 107 | −10.198 | −16.891 | 129.625 | 1.00 | 65.53 | A | O |
| ATOM | 36 | C | SER | 107 | −12.776 | −17.828 | 129.360 | 1.00 | 64.86 | A | C |
| ATOM | 37 | O | SER | 107 | −13.087 | −18.721 | 128.573 | 1.00 | 64.79 | A | O |
| ATOM | 38 | N | LYS | 108 | −13.074 | −16.549 | 129.154 | 1.00 | 63.49 | A | N |
| ATOM | 39 | CA | LYS | 108 | −13.772 | −16.121 | 127.948 | 1.00 | 62.43 | A | C |
| ATOM | 40 | CB | LYS | 108 | −14.196 | −14.650 | 128.055 | 1.00 | 62.56 | A | C |
| ATOM | 41 | CG | LYS | 108 | −15.668 | −14.437 | 128.417 | 1.00 | 62.85 | A | C |
| ATOM | 42 | CD | LYS | 108 | −16.022 | −15.032 | 129.776 | 1.00 | 63.11 | A | C |
| ATOM | 43 | CE | LYS | 108 | −17.482 | −14.777 | 130.129 | 1.00 | 63.62 | A | C |
| ATOM | 44 | NZ | LYS | 108 | −17.861 | −15.362 | 131.449 | 1.00 | 63.58 | A | N |
| ATOM | 45 | C | LYS | 108 | −12.848 | −16.305 | 126.750 | 1.00 | 61.42 | A | C |
| ATOM | 46 | O | LYS | 108 | −13.289 | −16.672 | 125.661 | 1.00 | 61.44 | A | O |
| ATOM | 47 | N | GLU | 109 | −11.563 | −16.047 | 126.959 | 1.00 | 60.15 | A | N |
| ATOM | 48 | CA | GLU | 109 | −10.580 | −16.204 | 125.900 | 1.00 | 58.91 | A | C |
| ATOM | 49 | CB | GLU | 109 | −9.232 | −15.655 | 126.358 | 1.00 | 59.90 | A | C |
| ATOM | 50 | CG | GLU | 109 | −8.171 | −15.661 | 125.279 | 1.00 | 61.96 | A | C |
| ATOM | 51 | CD | GLU | 109 | −6.868 | −15.046 | 125.745 | 1.00 | 63.27 | A | C |
| ATOM | 52 | OE1 | GLU | 109 | −6.885 | −13.866 | 126.160 | 1.00 | 64.16 | A | O |
| ATOM | 53 | OE2 | GLU | 109 | −5.829 | −15.741 | 125.696 | 1.00 | 63.84 | A | O |
| ATOM | 54 | C | GLU | 109 | −10.443 | −17.682 | 125.524 | 1.00 | 57.30 | A | C |
| ATOM | 55 | O | GLU | 109 | −10.154 | −18.014 | 124.376 | 1.00 | 56.66 | A | O |
| ATOM | 56 | N | GLN | 110 | −10.655 | −18.560 | 126.499 | 1.00 | 55.60 | A | N |
| ATOM | 57 | CA | GLN | 110 | −10.564 | −19.997 | 126.284 | 1.00 | 54.48 | A | C |
| ATOM | 58 | CB | GLN | 110 | −10.456 | −20.723 | 127.626 | 1.00 | 53.38 | A | C |
| ATOM | 59 | CG | GLN | 110 | −9.118 | −20.512 | 128.310 | 1.00 | 52.62 | A | C |
| ATOM | 60 | CD | GLN | 110 | −9.004 | −21.225 | 129.642 | 1.00 | 52.04 | A | C |
| ATOM | 61 | OE1 | GLN | 110 | −7.901 | −21.441 | 130.141 | 1.00 | 51.99 | A | O |
| ATOM | 62 | NE2 | GLN | 110 | −10.141 | −21.583 | 130.230 | 1.00 | 51.70 | A | N |
| ATOM | 63 | C | GLN | 110 | −11.754 | −20.537 | 125.503 | 1.00 | 54.10 | A | C |
| ATOM | 64 | O | GLN | 110 | −11.603 | −21.426 | 124.671 | 1.00 | 53.77 | A | O |
| ATOM | 65 | N | GLU | 111 | −12.938 | −20.001 | 125.772 | 1.00 | 53.80 | A | N |
| ATOM | 66 | CA | GLU | 111 | −14.130 | −20.450 | 125.068 | 1.00 | 53.73 | A | C |
| ATOM | 67 | CB | GLU | 111 | −15.389 | −19.943 | 125.774 | 1.00 | 54.85 | A | C |
| ATOM | 68 | CG | GLU | 111 | −15.607 | −20.597 | 127.131 | 1.00 | 56.90 | A | C |
| ATOM | 69 | CD | GLU | 111 | −16.899 | −20.172 | 127.793 | 1.00 | 58.68 | A | C |
| ATOM | 70 | OE1 | GLU | 111 | −17.970 | −20.349 | 127.171 | 1.00 | 60.00 | A | O |
| ATOM | 71 | OE2 | GLU | 111 | −16.846 | −19.666 | 128.936 | 1.00 | 59.57 | A | O |
| ATOM | 72 | C | GLU | 111 | −14.112 | −20.007 | 123.610 | 1.00 | 52.44 | A | C |
| ATOM | 73 | O | GLU | 111 | −14.672 | −20.680 | 122.747 | 1.00 | 52.44 | A | O |
| ATOM | 74 | N | GLU | 112 | −13.464 | −18.880 | 123.334 | 1.00 | 50.93 | A | N |
| ATOM | 75 | CA | GLU | 112 | −13.367 | −18.387 | 121.968 | 1.00 | 49.46 | A | C |
| ATOM | 76 | CB | GLU | 112 | −12.980 | −16.909 | 121.956 | 1.00 | 50.56 | A | C |
| ATOM | 77 | CG | GLU | 112 | −14.044 | −16.030 | 121.322 | 1.00 | 52.74 | A | C |
| ATOM | 78 | CD | GLU | 112 | −15.427 | −16.308 | 121.887 | 1.00 | 53.94 | A | C |
| ATOM | 79 | OE1 | GLU | 112 | −15.634 | −16.105 | 123.106 | 1.00 | 54.84 | A | O |
| ATOM | 80 | OE2 | GLU | 112 | −16.306 | −16.737 | 121.110 | 1.00 | 54.87 | A | O |
| ATOM | 81 | C | GLU | 112 | −12.332 | −19.204 | 121.212 | 1.00 | 47.53 | A | C |
| ATOM | 82 | O | GLU | 112 | −12.470 | −19.442 | 120.015 | 1.00 | 47.52 | A | O |
| ATOM | 83 | N | LEU | 113 | −11.290 | −19.622 | 121.922 | 1.00 | 45.16 | A | N |
| ATOM | 84 | CA | LEU | 113 | −10.236 | −20.440 | 121.341 | 1.00 | 42.62 | A | C |
| ATOM | 85 | CB | LEU | 113 | −9.217 | −20.822 | 122.418 | 1.00 | 42.09 | A | C |
| ATOM | 86 | CG | LEU | 113 | −7.813 | −21.290 | 122.021 | 1.00 | 42.04 | A | C |
| ATOM | 87 | CD1 | LEU | 113 | −7.183 | −21.991 | 123.223 | 1.00 | 41.08 | A | C |
| ATOM | 88 | CD2 | LEU | 113 | −7.861 | −22.234 | 120.836 | 1.00 | 41.07 | A | C |
| ATOM | 89 | C | LEU | 113 | −10.916 | −21.704 | 120.824 | 1.00 | 41.07 | A | C |
| ATOM | 90 | O | LEU | 113 | −10.746 | −22.096 | 119.670 | 1.00 | 39.68 | A | O |
| ATOM | 91 | N | ILE | 114 | −11.691 | −22.327 | 121.706 | 1.00 | 39.49 | A | N |
| ATOM | 92 | CA | ILE | 114 | −12.416 | −23.548 | 121.395 | 1.00 | 39.05 | A | C |
| ATOM | 93 | CB | ILE | 114 | −13.126 | −24.082 | 122.660 | 1.00 | 37.48 | A | C |
| ATOM | 94 | CG2 | ILE | 114 | −13.999 | −25.273 | 122.313 | 1.00 | 37.05 | A | C |
| ATOM | 95 | CG1 | ILE | 114 | −12.075 | −24.469 | 123.709 | 1.00 | 36.78 | A | C |
| ATOM | 96 | CD1 | ILE | 114 | −12.648 | −24.927 | 125.034 | 1.00 | 35.81 | A | C |
| ATOM | 97 | C | ILE | 114 | −13.431 | −23.352 | 120.267 | 1.00 | 39.39 | A | C |
| ATOM | 98 | O | ILE | 114 | −13.632 | −24.240 | 119.440 | 1.00 | 39.39 | A | O |
| ATOM | 99 | N | ARG | 115 | −14.069 | −22.190 | 120.229 | 1.00 | 39.79 | A | N |
| ATOM | 100 | CA | ARG | 115 | −15.045 | −21.913 | 119.185 | 1.00 | 40.43 | A | C |
| ATOM | 101 | CB | ARG | 115 | −15.769 | −20.598 | 119.473 | 1.00 | 42.33 | A | C |
| ATOM | 102 | CG | ARG | 115 | −16.842 | −20.248 | 118.451 | 1.00 | 45.85 | A | C |
| ATOM | 103 | CD | ARG | 115 | −16.819 | −18.762 | 118.113 | 1.00 | 49.10 | A | C |
| ATOM | 104 | NE | ARG | 115 | −15.561 | −18.376 | 117.470 | 1.00 | 51.76 | A | N |
| ATOM | 105 | CZ | ARG | 115 | −15.271 | −17.146 | 117.053 | 1.00 | 52.78 | A | C |
| ATOM | 106 | NH1 | ARG | 115 | −16.148 | −16.163 | 117.207 | 1.00 | 53.95 | A | N |
| ATOM | 107 | NH2 | ARG | 115 | −14.100 | −16.899 | 116.478 | 1.00 | 53.49 | A | N |
| ATOM | 108 | C | ARG | 115 | −14.327 | −21.824 | 117.839 | 1.00 | 39.30 | A | C |
| ATOM | 109 | O | ARG | 115 | −14.794 | −22.357 | 116.833 | 1.00 | 39.26 | A | O |
| ATOM | 110 | N | THR | 116 | −13.190 | −21.140 | 117.831 | 1.00 | 37.95 | A | N |
| ATOM | 111 | CA | THR | 116 | −12.389 | −20.979 | 116.626 | 1.00 | 36.97 | A | C |
| ATOM | 112 | CB | THR | 116 | −11.177 | −20.076 | 116.900 | 1.00 | 37.51 | A | C |
| ATOM | 113 | OG1 | THR | 116 | −11.625 | −18.847 | 117.483 | 1.00 | 39.12 | A | O |
| ATOM | 114 | CG2 | THR | 116 | −10.434 | −19.778 | 115.614 | 1.00 | 37.41 | A | C |
| ATOM | 115 | C | THR | 116 | −11.887 | −22.332 | 116.122 | 1.00 | 35.58 | A | C |
| ATOM | 116 | O | THR | 116 | −11.905 | −22.599 | 114.921 | 1.00 | 35.61 | A | O |
| ATOM | 117 | N | LEU | 117 | −11.434 | −23.176 | 117.046 | 1.00 | 33.47 | A | N |
| ATOM | 118 | CA | LEU | 117 | −10.932 | −24.500 | 116.705 | 1.00 | 31.78 | A | C |
| ATOM | 119 | CB | LEU | 117 | −10.286 | −25.143 | 117.929 | 1.00 | 30.67 | A | C |
| ATOM | 120 | CG | LEU | 117 | −8.959 | −24.582 | 118.426 | 1.00 | 30.04 | A | C |
| ATOM | 121 | CD1 | LEU | 117 | −8.543 | −25.311 | 119.688 | 1.00 | 29.00 | A | C |
| ATOM | 122 | CD2 | LEU | 117 | −7.905 | −24.735 | 117.345 | 1.00 | 30.20 | A | C |
| ATOM | 123 | C | LEU | 117 | −12.041 | −25.413 | 116.187 | 1.00 | 31.49 | A | C |
| ATOM | 124 | O | LEU | 117 | −11.864 | −26.112 | 115.195 | 1.00 | 31.28 | A | O |
| ATOM | 125 | N | LEU | 118 | −13.179 | −25.413 | 116.876 | 1.00 | 31.24 | A | N |
| ATOM | 126 | CA | LEU | 118 | −14.320 | −26.233 | 116.487 | 1.00 | 30.90 | A | C |
| ATOM | 127 | CB | LEU | 118 | −15.444 | −26.091 | 117.510 | 1.00 | 30.93 | A | C |
| ATOM | 128 | CG | LEU | 118 | −15.173 | −26.707 | 118.882 | 1.00 | 31.21 | A | C |
| ATOM | 129 | CD1 | LEU | 118 | −16.333 | −26.391 | 119.819 | 1.00 | 31.43 | A | C |
| ATOM | 130 | CD2 | LEU | 118 | −14.987 | −28.210 | 118.737 | 1.00 | 30.10 | A | C |
| ATOM | 131 | C | LEU | 118 | −14.841 | −25.848 | 115.111 | 1.00 | 30.73 | A | C |
| ATOM | 132 | O | LEU | 118 | −15.126 | −26.713 | 114.287 | 1.00 | 30.25 | A | O |
| ATOM | 133 | N | GLY | 119 | −14.963 | −24.544 | 114.872 | 1.00 | 30.45 | A | N |
| ATOM | 134 | CA | GLY | 119 | −15.444 | −24.067 | 113.586 | 1.00 | 29.84 | A | C |
| ATOM | 135 | C | GLY | 119 | −14.551 | −24.519 | 112.445 | 1.00 | 29.41 | A | C |
| ATOM | 136 | O | GLY | 119 | −15.036 | −24.986 | 111.411 | 1.00 | 29.26 | A | O |
| ATOM | 137 | N | ALA | 120 | −13.242 | −24.383 | 112.634 | 1.00 | 28.27 | A | N |
| ATOM | 138 | CA | ALA | 120 | −12.277 | −24.791 | 111.623 | 1.00 | 27.50 | A | C |
| ATOM | 139 | CB | ALA | 120 | −10.887 | −24.294 | 112.006 | 1.00 | 28.11 | A | C |
| ATOM | 140 | C | ALA | 120 | −12.273 | −26.317 | 111.455 | 1.00 | 26.73 | A | C |
| ATOM | 141 | O | ALA | 120 | −12.223 | −26.826 | 110.336 | 1.00 | 26.07 | A | O |
| ATOM | 142 | N | HIS | 121 | −12.348 | −27.038 | 112.569 | 1.00 | 26.34 | A | N |
| ATOM | 143 | CA | HIS | 121 | −12.356 | −28.498 | 112.542 | 1.00 | 25.51 | A | C |
| ATOM | 144 | CB | HIS | 121 | −12.250 | −29.053 | 113.967 | 1.00 | 25.42 | A | C |
| ATOM | 145 | CG | HIS | 121 | −12.478 | −30.531 | 114.058 | 1.00 | 25.78 | A | C |
| ATOM | 146 | CD2 | HIS | 121 | −11.622 | −31.573 | 113.949 | 1.00 | 25.53 | A | C |
| ATOM | 147 | ND1 | HIS | 121 | −13.729 | −31.082 | 114.240 | 1.00 | 26.55 | A | N |
| ATOM | 148 | CE1 | HIS | 121 | −13.633 | −32.398 | 114.239 | 1.00 | 27.01 | A | C |
| ATOM | 149 | NE2 | HIS | 121 | −12.364 | −32.723 | 114.064 | 1.00 | 27.07 | A | N |
| ATOM | 150 | C | HIS | 121 | −13.595 | −29.068 | 111.857 | 1.00 | 25.65 | A | C |
| ATOM | 151 | O | HIS | 121 | −13.491 | −29.943 | 111.000 | 1.00 | 23.32 | A | O |
| ATOM | 152 | N | THR | 122 | −14.769 | −28.572 | 112.233 | 1.00 | 26.18 | A | N |
| ATOM | 153 | CA | THR | 122 | −16.013 | −29.054 | 111.644 | 1.00 | 27.73 | A | C |
| ATOM | 154 | CB | THR | 122 | −17.241 | −28.391 | 112.310 | 1.00 | 27.99 | A | C |
| ATOM | 155 | OG1 | THR | 122 | −17.135 | −26.970 | 112.194 | 1.00 | 32.40 | A | O |
| ATOM | 156 | CG2 | THR | 122 | −17.319 | −28.750 | 113.780 | 1.00 | 27.74 | A | C |
| ATOM | 157 | C | THR | 122 | −16.063 | −28.799 | 110.137 | 1.00 | 27.54 | A | C |
| ATOM | 158 | O | THR | 122 | −16.490 | −29.657 | 109.368 | 1.00 | 26.85 | A | O |
| ATOM | 159 | N | ARG | 123 | −15.598 | −27.627 | 109.715 | 1.00 | 28.07 | A | N |
| ATOM | 160 | CA | ARG | 123 | −15.612 | −27.269 | 108.300 | 1.00 | 29.18 | A | C |
| ATOM | 161 | CB | ARG | 123 | −15.349 | −25.762 | 108.141 | 1.00 | 29.82 | A | C |
| ATOM | 162 | CG | ARG | 123 | −15.610 | −25.226 | 106.727 | 1.00 | 33.27 | A | C |
| ATOM | 163 | CD | ARG | 123 | −15.159 | −23.765 | 106.536 | 1.00 | 34.42 | A | C |
| ATOM | 164 | NE | ARG | 123 | −16.031 | −22.773 | 107.179 | 1.00 | 36.89 | A | N |
| ATOM | 165 | CZ | ARG | 123 | −17.220 | −22.381 | 106.714 | 1.00 | 36.96 | A | C |
| ATOM | 166 | NH1 | ARG | 123 | −17.715 | −22.888 | 105.592 | 1.00 | 37.38 | A | N |
| ATOM | 167 | NH2 | ARG | 123 | −17.913 | −21.458 | 107.366 | 1.00 | 37.20 | A | N |
| ATOM | 168 | C | ARG | 123 | −14.628 | −28.055 | 107.415 | 1.00 | 28.35 | A | C |
| ATOM | 169 | O | ARG | 123 | −14.967 | −28.431 | 106.290 | 1.00 | 27.61 | A | O |
| ATOM | 170 | N | HIS | 124 | −13.426 | −28.324 | 107.923 | 1.00 | 27.75 | A | N |
| ATOM | 171 | CA | HIS | 124 | −12.409 | −29.016 | 107.125 | 1.00 | 27.66 | A | C |
| ATOM | 172 | CB | HIS | 124 | −11.148 | −28.147 | 107.062 | 1.00 | 28.26 | A | C |
| ATOM | 173 | CG | HIS | 124 | −11.395 | −26.764 | 106.543 | 1.00 | 29.25 | A | C |
| ATOM | 174 | CD2 | HIS | 124 | −11.945 | −26.333 | 105.382 | 1.00 | 28.40 | A | C |
| ATOM | 175 | ND1 | HIS | 124 | −11.081 | −25.631 | 107.263 | 1.00 | 29.27 | A | N |
| ATOM | 176 | CE1 | HIS | 124 | −11.426 | −24.562 | 106.567 | 1.00 | 28.76 | A | C |
| ATOM | 177 | NE2 | HIS | 124 | −11.953 | −24.960 | 105.423 | 1.00 | 29.33 | A | N |
| ATOM | 178 | C | HIS | 124 | −11.982 | −30.448 | 107.478 | 1.00 | 26.91 | A | C |
| ATOM | 179 | O | HIS | 124 | −11.534 | −31.189 | 106.599 | 1.00 | 26.66 | A | O |
| ATOM | 180 | N | MET | 125 | −12.118 | −30.855 | 108.735 | 1.00 | 26.01 | A | N |
| ATOM | 181 | CA | MET | 125 | −11.659 | −32.193 | 109.108 | 1.00 | 26.25 | A | C |
| ATOM | 182 | CB | MET | 125 | −10.443 | −32.063 | 110.025 | 1.00 | 26.44 | A | C |
| ATOM | 183 | CG | MET | 125 | −9.325 | −31.218 | 109.424 | 1.00 | 27.40 | A | C |
| ATOM | 184 | SD | MET | 125 | −7.795 | −31.333 | 110.350 | 1.00 | 31.87 | A | S |
| ATOM | 185 | CE | MET | 125 | −8.358 | −30.864 | 111.998 | 1.00 | 31.61 | A | C |
| ATOM | 186 | C | MET | 125 | −12.657 | −33.158 | 109.731 | 1.00 | 25.47 | A | C |
| ATOM | 187 | O | MET | 125 | −12.621 | −34.355 | 109.446 | 1.00 | 25.35 | A | O |
| ATOM | 188 | N | GLY | 126 | −13.536 | −32.641 | 110.581 | 1.00 | 24.84 | A | N |
| ATOM | 189 | CA | GLY | 126 | −14.524 | −33.471 | 111.247 | 1.00 | 24.24 | A | C |
| ATOM | 190 | C | GLY | 126 | −15.123 | −34.597 | 110.426 | 1.00 | 23.67 | A | C |
| ATOM | 191 | O | GLY | 126 | −15.172 | −35.739 | 110.883 | 1.00 | 23.66 | A | O |
| ATOM | 192 | N | THR | 127 | −15.581 | −34.297 | 109.215 | 1.00 | 22.39 | A | N |
| ATOM | 193 | CA | THR | 127 | −16.177 | −35.339 | 108.390 | 1.00 | 22.17 | A | C |
| ATOM | 194 | CB | THR | 127 | −17.667 | −35.039 | 108.101 | 1.00 | 21.86 | A | C |
| ATOM | 195 | OG1 | THR | 127 | −17.787 | −33.751 | 107.497 | 1.00 | 21.82 | A | O |
| ATOM | 196 | CG2 | THR | 127 | −18.483 | −35.056 | 109.387 | 1.00 | 22.67 | A | C |
| ATOM | 197 | C | THR | 127 | −15.463 | −35.571 | 107.067 | 1.00 | 21.87 | A | C |
| ATOM | 198 | O | THR | 127 | −16.065 | −36.071 | 106.118 | 1.00 | 21.50 | A | O |
| ATOM | 199 | N | MET | 128 | −14.179 | −35.232 | 106.996 | 1.00 | 21.50 | A | N |
| ATOM | 200 | CA | MET | 128 | −13.446 | −35.420 | 105.746 | 1.00 | 21.52 | A | C |
| ATOM | 201 | CB | MET | 128 | −12.031 | −34.823 | 105.845 | 1.00 | 22.11 | A | C |
| ATOM | 202 | CG | MET | 128 | −11.061 | −35.562 | 106.770 | 1.00 | 22.14 | A | C |
| ATOM | 203 | SD | MET | 128 | −9.438 | −34.750 | 106.857 | 1.00 | 21.82 | A | S |
| ATOM | 204 | CE | MET | 128 | −8.599 | −35.832 | 108.065 | 1.00 | 22.47 | A | C |
| ATOM | 205 | C | MET | 128 | −13.361 | −36.881 | 105.315 | 1.00 | 21.38 | A | C |
| ATOM | 206 | O | MET | 128 | −13.211 | −37.170 | 104.131 | 1.00 | 21.52 | A | O |
| ATOM | 207 | N | PHE | 129 | −13.463 | −37.806 | 106.265 | 1.00 | 21.94 | A | N |
| ATOM | 208 | CA | PHE | 129 | −13.399 | −39.231 | 105.939 | 1.00 | 21.94 | A | C |
| ATOM | 209 | CB | PHE | 129 | −13.509 | −40.080 | 107.219 | 1.00 | 21.34 | A | C |
| ATOM | 210 | CG | PHE | 129 | −14.896 | −40.130 | 107.811 | 1.00 | 21.67 | A | C |
| ATOM | 211 | CD1 | PHE | 129 | −15.849 | −41.026 | 107.322 | 1.00 | 21.16 | A | C |
| ATOM | 212 | CD2 | PHE | 129 | −15.251 | −39.284 | 108.855 | 1.00 | 20.50 | A | C |
| ATOM | 213 | CE1 | PHE | 129 | −17.137 | −41.077 | 107.869 | 1.00 | 21.61 | A | C |
| ATOM | 214 | CE2 | PHE | 129 | −16.533 | −39.327 | 109.406 | 1.00 | 21.65 | A | C |
| ATOM | 215 | CZ | PHE | 129 | −17.477 | −40.225 | 108.912 | 1.00 | 21.57 | A | C |
| ATOM | 216 | C | PHE | 129 | −14.484 | −39.644 | 104.938 | 1.00 | 21.50 | A | C |
| ATOM | 217 | O | PHE | 129 | −14.315 | −40.613 | 104.197 | 1.00 | 20.88 | A | O |
| ATOM | 218 | N | GLU | 130 | −15.589 | −38.906 | 104.911 | 1.00 | 21.77 | A | N |
| ATOM | 219 | CA | GLU | 130 | −16.686 | −39.207 | 103.996 | 1.00 | 23.20 | A | C |
| ATOM | 220 | CB | GLU | 130 | −17.886 | −38.307 | 104.298 | 1.00 | 23.75 | A | C |
| ATOM | 221 | CG | GLU | 130 | −18.476 | −38.533 | 105.681 | 1.00 | 26.72 | A | C |
| ATOM | 222 | CD | GLU | 130 | −19.666 | −37.630 | 105.968 | 1.00 | 27.23 | A | C |
| ATOM | 223 | OE1 | GLU | 130 | −19.938 | −36.721 | 105.154 | 1.00 | 27.13 | A | O |
| ATOM | 224 | OE2 | GLU | 130 | −20.321 | −37.830 | 107.014 | 1.00 | 28.12 | A | O |
| ATOM | 225 | C | GLU | 130 | −16.313 | −39.072 | 102.519 | 1.00 | 22.81 | A | C |
| ATOM | 226 | O | GLU | 130 | −17.020 | −39.581 | 101.648 | 1.00 | 22.98 | A | O |
| ATOM | 227 | N | GLN | 131 | −15.211 | −38.396 | 102.225 | 1.00 | 22.34 | A | N |
| ATOM | 228 | CA | GLN | 131 | −14.826 | −38.251 | 100.834 | 1.00 | 23.67 | A | C |
| ATOM | 229 | CB | GLN | 131 | −14.212 | −36.864 | 100.579 | 1.00 | 25.71 | A | C |
| ATOM | 230 | CG | GLN | 131 | −14.915 | −35.665 | 101.279 | 1.00 | 31.91 | A | C |
| ATOM | 231 | CD | GLN | 131 | −16.421 | −35.495 | 100.986 | 1.00 | 35.53 | A | C |
| ATOM | 232 | OE1 | GLN | 131 | −17.020 | −34.490 | 101.382 | 1.00 | 39.09 | A | O |
| ATOM | 233 | NE2 | GLN | 131 | −17.033 | −36.465 | 100.314 | 1.00 | 37.15 | A | N |
| ATOM | 234 | C | GLN | 131 | −13.871 | −39.344 | 100.350 | 1.00 | 22.82 | A | C |
| ATOM | 235 | O | GLN | 131 | −13.486 | −39.350 | 99.186 | 1.00 | 22.74 | A | O |
| ATOM | 236 | N | PHE | 132 | −13.500 | −40.274 | 101.229 | 1.00 | 21.59 | A | N |
| ATOM | 237 | CA | PHE | 132 | −12.585 | −41.345 | 100.840 | 1.00 | 20.93 | A | C |
| ATOM | 238 | CB | PHE | 132 | −12.287 | −42.289 | 102.023 | 1.00 | 19.90 | A | C |
| ATOM | 239 | CG | PHE | 132 | −11.445 | −41.667 | 103.133 | 1.00 | 19.86 | A | C |
| ATOM | 240 | CD1 | PHE | 132 | −10.858 | −40.409 | 102.982 | 1.00 | 19.07 | A | C |
| ATOM | 241 | CD2 | PHE | 132 | −11.258 | −42.347 | 104.337 | 1.00 | 18.20 | A | C |
| ATOM | 242 | CE1 | PHE | 132 | −10.104 | −39.834 | 104.010 | 1.00 | 18.72 | A | C |
| ATOM | 243 | CE2 | PHE | 132 | −10.507 | −41.783 | 105.371 | 1.00 | 18.68 | A | C |
| ATOM | 244 | CZ | PHE | 132 | −9.928 | −40.525 | 105.211 | 1.00 | 18.24 | A | C |
| ATOM | 245 | C | PHE | 132 | −13.119 | −42.169 | 99.658 | 1.00 | 20.71 | A | C |
| ATOM | 246 | O | PHE | 132 | −12.330 | −42.675 | 98.861 | 1.00 | 20.20 | A | O |
| ATOM | 247 | N | VAL | 133 | −14.442 | −42.300 | 99.538 | 1.00 | 20.69 | A | N |
| ATOM | 248 | CA | VAL | 133 | −15.034 | −43.076 | 98.438 | 1.00 | 22.08 | A | C |
| ATOM | 249 | CB | VAL | 133 | −16.554 | −43.305 | 98.625 | 1.00 | 22.06 | A | C |
| ATOM | 250 | CG1 | VAL | 133 | −16.799 | −44.205 | 99.820 | 1.00 | 22.30 | A | C |
| ATOM | 251 | CG2 | VAL | 133 | −17.281 | −41.975 | 98.794 | 1.00 | 20.84 | A | C |
| ATOM | 252 | C | VAL | 133 | −14.825 | −42.461 | 97.056 | 1.00 | 23.30 | A | C |
| ATOM | 253 | O | VAL | 133 | −15.065 | −43.110 | 96.040 | 1.00 | 21.98 | A | O |
| ATOM | 254 | N | GLN | 134 | −14.370 | −41.214 | 97.028 | 1.00 | 25.25 | A | N |
| ATOM | 255 | CA | GLN | 134 | −14.110 | −40.505 | 95.786 | 1.00 | 28.42 | A | C |
| ATOM | 256 | CB | GLN | 134 | −14.265 | −39.001 | 95.992 | 1.00 | 31.35 | A | C |
| ATOM | 257 | CG | GLN | 134 | −15.676 | −38.496 | 96.138 | 1.00 | 35.93 | A | C |
| ATOM | 258 | CD | GLN | 134 | −15.692 | −37.018 | 96.459 | 1.00 | 38.86 | A | C |
| ATOM | 259 | OE1 | GLN | 134 | −14.978 | −36.224 | 95.835 | 1.00 | 40.75 | A | O |
| ATOM | 260 | NE2 | GLN | 134 | −16.510 | −36.634 | 97.427 | 1.00 | 40.53 | A | N |
| ATOM | 261 | C | GLN | 134 | −12.701 | −40.739 | 95.264 | 1.00 | 28.55 | A | C |
| ATOM | 262 | O | GLN | 134 | −12.305 | −40.113 | 94.281 | 1.00 | 28.59 | A | O |
| ATOM | 263 | N | PHE | 135 | −11.933 | −41.612 | 95.911 | 1.00 | 27.76 | A | N |
| ATOM | 264 | CA | PHE | 135 | −10.562 | −41.834 | 95.464 | 1.00 | 27.25 | A | C |
| ATOM | 265 | CB | PHE | 135 | −9.593 | −41.361 | 96.556 | 1.00 | 27.33 | A | C |
| ATOM | 266 | CG | PHE | 135 | −9.653 | −39.872 | 96.806 | 1.00 | 27.16 | A | C |
| ATOM | 267 | CD1 | PHE | 135 | −9.062 | −38.978 | 95.916 | 1.00 | 27.07 | A | C |
| ATOM | 268 | CD2 | PHE | 135 | −10.346 | −39.363 | 97.900 | 1.00 | 27.37 | A | C |
| ATOM | 269 | CE1 | PHE | 135 | −9.163 | −37.595 | 96.110 | 1.00 | 26.82 | A | C |
| ATOM | 270 | CE2 | PHE | 135 | −10.455 | −37.979 | 98.104 | 1.00 | 27.26 | A | C |
| ATOM | 271 | CZ | PHE | 135 | −9.861 | −37.096 | 97.206 | 1.00 | 26.50 | A | C |
| ATOM | 272 | C | PHE | 135 | −10.241 | −43.256 | 95.022 | 1.00 | 26.66 | A | C |
| ATOM | 273 | O | PHE | 135 | −9.247 | −43.843 | 95.444 | 1.00 | 26.40 | A | O |
| ATOM | 274 | N | ARG | 136 | −11.086 | −43.784 | 94.143 | 1.00 | 26.15 | A | N |
| ATOM | 275 | CA | ARG | 136 | −10.934 | −45.129 | 93.586 | 1.00 | 25.96 | A | C |
| ATOM | 276 | CB | ARG | 136 | −9.900 | −45.115 | 92.452 | 1.00 | 26.56 | A | C |
| ATOM | 277 | CG | ARG | 136 | −10.158 | −44.063 | 91.372 | 1.00 | 29.01 | A | C |
| ATOM | 278 | CD | ARG | 136 | −8.988 | −43.086 | 91.285 | 1.00 | 31.35 | A | C |
| ATOM | 279 | NE | ARG | 136 | −9.452 | −41.743 | 90.949 | 1.00 | 34.32 | A | N |
| ATOM | 280 | CZ | ARG | 136 | −8.939 | −40.621 | 91.442 | 1.00 | 35.38 | A | C |
| ATOM | 281 | NH1 | ARG | 136 | −7.934 | −40.660 | 92.305 | 1.00 | 34.41 | A | N |
| ATOM | 282 | NH2 | ARG | 136 | −9.442 | −39.452 | 91.073 | 1.00 | 37.45 | A | N |
| ATOM | 283 | C | ARG | 136 | −10.530 | −46.179 | 94.623 | 1.00 | 25.24 | A | C |
| ATOM | 284 | O | ARG | 136 | −9.486 | −46.819 | 94.496 | 1.00 | 24.38 | A | O |
| ATOM | 285 | N | PRO | 137 | −11.357 | −46.371 | 95.662 | 1.00 | 24.16 | A | N |
| ATOM | 286 | CD | PRO | 137 | −12.632 | −45.707 | 95.999 | 1.00 | 23.61 | A | C |
| ATOM | 287 | CA | PRO | 137 | −11.008 | −47.366 | 96.672 | 1.00 | 23.41 | A | C |
| ATOM | 288 | CB | PRO | 137 | −11.999 | −47.081 | 97.790 | 1.00 | 23.30 | A | C |
| ATOM | 289 | CG | PRO | 137 | −13.229 | −46.654 | 97.013 | 1.00 | 23.80 | A | C |
| ATOM | 290 | C | PRO | 137 | −11.164 | −48.776 | 96.149 | 1.00 | 23.31 | A | C |
| ATOM | 291 | O | PRO | 137 | −12.181 | −49.115 | 95.545 | 1.00 | 22.63 | A | O |
| ATOM | 292 | N | PRO | 138 | −10.147 | −49.620 | 96.358 | 1.00 | 23.12 | A | N |
| ATOM | 293 | CD | PRO | 138 | −8.801 | −49.369 | 96.907 | 1.00 | 23.19 | A | C |
| ATOM | 294 | CA | PRO | 138 | −10.283 | −50.993 | 95.875 | 1.00 | 23.26 | A | C |
| ATOM | 295 | CB | PRO | 138 | −9.045 | −51.679 | 96.450 | 1.00 | 23.98 | A | C |
| ATOM | 296 | CG | PRO | 138 | −8.015 | −50.578 | 96.421 | 1.00 | 24.11 | A | C |
| ATOM | 297 | C | PRO | 138 | −11.585 | −51.547 | 96.464 | 1.00 | 22.75 | A | C |
| ATOM | 298 | O | PRO | 138 | −12.000 | −51.142 | 97.556 | 1.00 | 22.46 | A | O |
| ATOM | 299 | N | ALA | 139 | −12.221 | −52.468 | 95.748 | 1.00 | 21.31 | A | N |
| ATOM | 300 | CA | ALA | 139 | −13.475 | −53.061 | 96.193 | 1.00 | 21.53 | A | C |
| ATOM | 301 | CB | ALA | 139 | −14.001 | −54.024 | 95.114 | 1.00 | 21.98 | A | C |
| ATOM | 302 | C | ALA | 139 | −13.442 | −53.774 | 97.556 | 1.00 | 21.59 | A | C |
| ATOM | 303 | O | ALA | 139 | −14.439 | −53.750 | 98.282 | 1.00 | 20.89 | A | O |
| ATOM | 304 | N | HIS | 140 | −12.320 | −54.405 | 97.916 | 1.00 | 21.14 | A | N |
| ATOM | 305 | CA | HIS | 140 | −12.253 | −55.107 | 99.199 | 1.00 | 21.89 | A | C |
| ATOM | 306 | CB | HIS | 140 | −10.941 | −55.903 | 99.342 | 1.00 | 22.63 | A | C |
| ATOM | 307 | CG | HIS | 140 | −9.759 | −55.062 | 99.725 | 1.00 | 21.19 | A | C |
| ATOM | 308 | CD2 | HIS | 140 | −9.227 | −54.774 | 100.936 | 1.00 | 21.22 | A | C |
| ATOM | 309 | ND1 | HIS | 140 | −9.024 | −54.347 | 98.804 | 1.00 | 20.38 | A | N |
| ATOM | 310 | CE1 | HIS | 140 | −8.094 | −53.650 | 99.431 | 1.00 | 21.24 | A | C |
| ATOM | 311 | NE2 | HIS | 140 | −8.196 | −53.890 | 100.726 | 1.00 | 22.01 | A | N |
| ATOM | 312 | C | HIS | 140 | −12.388 | −54.153 | 100.392 | 1.00 | 22.47 | A | C |
| ATOM | 313 | O | HIS | 140 | −12.605 | −54.586 | 101.518 | 1.00 | 22.16 | A | O |
| ATOM | 314 | N | LEU | 141 | −12.251 | −52.857 | 100.134 | 1.00 | 23.58 | A | N |
| ATOM | 315 | CA | LEU | 141 | −12.364 | −51.827 | 101.166 | 1.00 | 23.85 | A | C |
| ATOM | 316 | CB | LEU | 141 | −11.777 | −50.520 | 100.634 | 1.00 | 23.18 | A | C |
| ATOM | 317 | CG | LEU | 141 | −10.527 | −49.937 | 101.294 | 1.00 | 24.27 | A | C |
| ATOM | 318 | CD1 | LEU | 141 | −9.667 | −51.037 | 101.903 | 1.00 | 22.26 | A | C |
| ATOM | 319 | CD2 | LEU | 141 | −9.766 | −49.121 | 100.262 | 1.00 | 21.25 | A | C |
| ATOM | 320 | C | LEU | 141 | −13.812 | −51.599 | 101.603 | 1.00 | 24.27 | A | C |
| ATOM | 321 | O | LEU | 141 | −14.066 | −51.148 | 102.718 | 1.00 | 23.20 | A | O |
| ATOM | 322 | N | PHE | 142 | −14.759 | −51.902 | 100.719 | 1.00 | 24.16 | A | N |
| ATOM | 323 | CA | PHE | 142 | −16.173 | −51.717 | 101.032 | 1.00 | 25.36 | A | C |
| ATOM | 324 | CB | PHE | 142 | −17.017 | −51.773 | 99.752 | 1.00 | 23.10 | A | C |
| ATOM | 325 | CG | PHE | 142 | −16.898 | −50.549 | 98.901 | 1.00 | 22.13 | A | C |
| ATOM | 326 | CD1 | PHE | 142 | −17.570 | −49.385 | 99.240 | 1.00 | 22.32 | A | C |
| ATOM | 327 | CD2 | PHE | 142 | −16.087 | −50.544 | 97.780 | 1.00 | 22.24 | A | C |
| ATOM | 328 | CE1 | PHE | 142 | −17.432 | −48.234 | 98.467 | 1.00 | 22.25 | A | C |
| ATOM | 329 | CE2 | PHE | 142 | −15.944 | −49.396 | 97.006 | 1.00 | 22.18 | A | C |
| ATOM | 330 | CZ | PHE | 142 | −16.615 | −48.242 | 97.349 | 1.00 | 20.96 | A | C |
| ATOM | 331 | C | PHE | 142 | −16.666 | −52.771 | 102.005 | 1.00 | 26.65 | A | C |
| ATOM | 332 | O | PHE | 142 | −16.213 | −53.914 | 101.976 | 1.00 | 26.20 | A | O |
| ATOM | 333 | N | ILE | 143 | −17.594 | −52.380 | 102.873 | 1.00 | 28.79 | A | N |
| ATOM | 334 | CA | ILE | 143 | −18.165 | −53.310 | 103.834 | 1.00 | 31.69 | A | C |
| ATOM | 335 | CB | ILE | 143 | −19.247 | −52.630 | 104.743 | 1.00 | 32.78 | A | C |
| ATOM | 336 | CG2 | ILE | 143 | −18.682 | −51.372 | 105.382 | 1.00 | 33.71 | A | C |
| ATOM | 337 | CG1 | ILE | 143 | −20.516 | −52.300 | 103.943 | 1.00 | 33.80 | A | C |
| ATOM | 338 | CD1 | ILE | 143 | −20.373 | −51.225 | 102.876 | 1.00 | 35.73 | A | C |
| ATOM | 339 | C | ILE | 143 | −18.814 | −54.449 | 103.039 | 1.00 | 32.40 | A | C |
| ATOM | 340 | O | ILE | 143 | −19.161 | −54.277 | 101.870 | 1.00 | 31.97 | A | O |
| ATOM | 341 | N | HIS | 144 | −18.967 | −55.606 | 103.672 | 1.00 | 33.54 | A | N |
| ATOM | 342 | CA | HIS | 144 | −19.568 | −56.769 | 103.023 | 1.00 | 35.74 | A | C |
| ATOM | 343 | CB | HIS | 144 | −20.924 | −56.405 | 102.398 | 1.00 | 36.54 | A | C |
| ATOM | 344 | CG | HIS | 144 | −21.853 | −55.688 | 103.330 | 1.00 | 37.08 | A | C |
| ATOM | 345 | CD2 | HIS | 144 | −22.508 | −54.509 | 103.201 | 1.00 | 36.90 | A | C |
| ATOM | 346 | ND1 | HIS | 144 | −22.207 | −56.189 | 104.563 | 1.00 | 37.27 | A | N |
| ATOM | 347 | CE1 | HIS | 144 | −23.037 | −55.349 | 105.156 | 1.00 | 37.23 | A | C |
| ATOM | 348 | NE2 | HIS | 144 | −23.235 | −54.321 | 104.350 | 1.00 | 37.35 | A | N |
| ATOM | 349 | C | HIS | 144 | −18.648 | −57.317 | 101.932 | 1.00 | 36.21 | A | C |
| ATOM | 350 | O | HIS | 144 | −19.113 | −57.751 | 100.877 | 1.00 | 36.04 | A | O |
| ATOM | 351 | N | HIS | 145 | −17.345 | −57.289 | 102.191 | 1.00 | 37.47 | A | N |
| ATOM | 352 | CA | HIS | 145 | −16.356 | −57.778 | 101.236 | 1.00 | 38.79 | A | C |
| ATOM | 353 | CB | HIS | 145 | −15.740 | −56.611 | 100.471 | 1.00 | 38.32 | A | C |
| ATOM | 354 | CG | HIS | 145 | −16.612 | −56.082 | 99.379 | 1.00 | 38.92 | A | C |
| ATOM | 355 | CD2 | HIS | 145 | −17.687 | −55.260 | 99.416 | 1.00 | 38.33 | A | C |
| ATOM | 356 | ND1 | HIS | 145 | −16.436 | −56.427 | 98.056 | 1.00 | 38.61 | A | N |
| ATOM | 357 | CE1 | HIS | 145 | −17.365 | −55.840 | 97.325 | 1.00 | 38.59 | A | C |
| ATOM | 358 | NE2 | HIS | 145 | −18.138 | −55.127 | 98.125 | 1.00 | 39.24 | A | N |
| ATOM | 359 | C | HIS | 145 | −15.248 | −58.564 | 101.914 | 1.00 | 39.68 | A | C |
| ATOM | 360 | O | HIS | 145 | −14.995 | −58.406 | 103.110 | 1.00 | 40.45 | A | O |
| ATOM | 361 | N | GLN | 146 | −14.593 | −59.417 | 101.134 | 1.00 | 40.62 | A | N |
| ATOM | 362 | CA | GLN | 146 | −13.495 | −60.232 | 101.632 | 1.00 | 40.83 | A | C |
| ATOM | 363 | CB | GLN | 146 | −13.470 | −61.585 | 100.899 | 1.00 | 42.96 | A | C |
| ATOM | 364 | CG | GLN | 146 | −13.528 | −61.487 | 99.376 | 1.00 | 46.15 | A | C |
| ATOM | 365 | CD | GLN | 146 | −13.498 | −62.850 | 98.685 | 1.00 | 48.68 | A | C |
| ATOM | 366 | OE1 | GLN | 146 | −14.422 | −63.659 | 98.824 | 1.00 | 49.88 | A | O |
| ATOM | 367 | NE2 | GLN | 146 | −12.430 | −63.105 | 97.934 | 1.00 | 49.37 | A | N |
| ATOM | 368 | C | GLN | 146 | −12.193 | −59.464 | 101.412 | 1.00 | 39.30 | A | C |
| ATOM | 369 | O | GLN | 146 | −12.075 | −58.685 | 100.467 | 1.00 | 39.67 | A | O |
| ATOM | 370 | N | PRO | 147 | −11.201 | −59.664 | 102.292 | 1.00 | 37.59 | A | N |
| ATOM | 371 | CD | PRO | 147 | −11.172 | −60.620 | 103.411 | 1.00 | 37.45 | A | C |
| ATOM | 372 | CA | PRO | 147 | −9.917 | −58.969 | 102.165 | 1.00 | 35.57 | A | C |
| ATOM | 373 | CB | PRO | 147 | −9.130 | −59.485 | 103.367 | 1.00 | 36.50 | A | C |
| ATOM | 374 | CG | PRO | 147 | −9.700 | −60.851 | 103.580 | 1.00 | 37.77 | A | C |
| ATOM | 375 | C | PRO | 147 | −9.198 | −59.208 | 100.835 | 1.00 | 33.38 | A | C |
| ATOM | 376 | O | PRO | 147 | −9.528 | −60.134 | 100.094 | 1.00 | 33.33 | A | O |
| ATOM | 377 | N | LEU | 148 | −8.227 | −58.353 | 100.535 | 1.00 | 30.98 | A | N |
| ATOM | 378 | CA | LEU | 148 | −7.448 | −58.458 | 99.305 | 1.00 | 29.30 | A | C |
| ATOM | 379 | CB | LEU | 148 | −6.282 | −57.465 | 99.323 | 1.00 | 29.16 | A | C |
| ATOM | 380 | CG | LEU | 148 | −6.117 | −56.372 | 98.263 | 1.00 | 29.82 | A | C |
| ATOM | 381 | CD1 | LEU | 148 | −4.665 | −55.910 | 98.289 | 1.00 | 29.71 | A | C |
| ATOM | 382 | CD2 | LEU | 148 | −6.466 | −56.870 | 96.883 | 1.00 | 29.27 | A | C |
| ATOM | 383 | C | LEU | 148 | −6.874 | −59.865 | 99.153 | 1.00 | 27.66 | A | C |
| ATOM | 384 | O | LEU | 148 | −6.281 | −60.406 | 100.085 | 1.00 | 26.44 | A | O |
| ATOM | 385 | N | PRO | 149 | −7.057 | −60.481 | 97.977 | 1.00 | 26.63 | A | N |
| ATOM | 386 | CD | PRO | 149 | −7.955 | −60.093 | 96.877 | 1.00 | 26.30 | A | C |
| ATOM | 387 | CA | PRO | 149 | −6.523 | −61.827 | 97.767 | 1.00 | 26.49 | A | C |
| ATOM | 388 | CB | PRO | 149 | −7.042 | −62.192 | 96.376 | 1.00 | 26.48 | A | C |
| ATOM | 389 | CG | PRO | 149 | −8.327 | −61.433 | 96.288 | 1.00 | 26.76 | A | C |
| ATOM | 390 | C | PRO | 149 | −4.993 | −61.798 | 97.824 | 1.00 | 26.14 | A | C |
| ATOM | 391 | O | PRO | 149 | −4.372 | −60.749 | 97.645 | 1.00 | 26.17 | A | O |
| ATOM | 392 | N | THR | 150 | −4.402 | −62.961 | 98.066 | 1.00 | 26.06 | A | N |
| ATOM | 393 | CA | THR | 150 | −2.955 | −63.130 | 98.167 | 1.00 | 25.41 | A | C |
| ATOM | 394 | CB | THR | 150 | −2.605 | −64.632 | 98.370 | 1.00 | 25.71 | A | C |
| ATOM | 395 | OG1 | THR | 150 | −2.981 | −65.037 | 99.694 | 1.00 | 23.28 | A | O |
| ATOM | 396 | CG2 | THR | 150 | −1.112 | −64.888 | 98.148 | 1.00 | 25.10 | A | C |
| ATOM | 397 | C | THR | 150 | −2.173 | −62.610 | 96.964 | 1.00 | 26.17 | A | C |
| ATOM | 398 | O | THR | 150 | −1.155 | −61.933 | 97.123 | 1.00 | 25.67 | A | O |
| ATOM | 399 | N | LEU | 151 | −2.652 | −62.924 | 95.766 | 1.00 | 26.09 | A | N |
| ATOM | 400 | CA | LEU | 151 | −1.973 | −62.520 | 94.543 | 1.00 | 27.02 | A | C |
| ATOM | 401 | CB | LEU | 151 | −1.976 | −63.692 | 93.548 | 1.00 | 28.10 | A | C |
| ATOM | 402 | CG | LEU | 151 | −0.752 | −64.622 | 93.469 | 1.00 | 28.68 | A | C |
| ATOM | 403 | CD1 | LEU | 151 | −0.153 | −64.874 | 94.830 | 1.00 | 28.89 | A | C |
| ATOM | 404 | CD2 | LEU | 151 | −1.171 | −65.927 | 92.809 | 1.00 | 28.50 | A | C |
| ATOM | 405 | C | LEU | 151 | −2.516 | −61.258 | 93.865 | 1.00 | 26.77 | A | C |
| ATOM | 406 | O | LEU | 151 | −1.978 | −60.827 | 92.849 | 1.00 | 26.56 | A | O |
| ATOM | 407 | N | ALA | 152 | −3.565 | −60.659 | 94.422 | 1.00 | 26.15 | A | N |
| ATOM | 408 | CA | ALA | 152 | −4.125 | −59.442 | 93.836 | 1.00 | 25.48 | A | C |
| ATOM | 409 | CB | ALA | 152 | −5.423 | −59.064 | 94.544 | 1.00 | 25.10 | A | C |
| ATOM | 410 | C | ALA | 152 | −3.121 | −58.290 | 93.937 | 1.00 | 24.90 | A | C |
| ATOM | 411 | O | ALA | 152 | −2.517 | −58.073 | 94.985 | 1.00 | 23.72 | A | O |
| ATOM | 412 | N | PRO | 153 | −2.921 | −57.544 | 92.841 | 1.00 | 24.54 | A | N |
| ATOM | 413 | CD | PRO | 153 | −3.389 | −57.751 | 91.463 | 1.00 | 25.59 | A | C |
| ATOM | 414 | CA | PRO | 153 | −1.968 | −56.435 | 92.919 | 1.00 | 24.98 | A | C |
| ATOM | 415 | CB | PRO | 153 | −1.970 | −55.859 | 91.495 | 1.00 | 25.27 | A | C |
| ATOM | 416 | CG | PRO | 153 | −3.229 | −56.374 | 90.870 | 1.00 | 25.95 | A | C |
| ATOM | 417 | C | PRO | 153 | −2.368 | −55.421 | 93.993 | 1.00 | 25.30 | A | C |
| ATOM | 418 | O | PRO | 153 | −3.554 | −55.182 | 94.220 | 1.00 | 25.69 | A | O |
| ATOM | 419 | N | VAL | 154 | −1.376 | −54.845 | 94.666 | 1.00 | 24.93 | A | N |
| ATOM | 420 | CA | VAL | 154 | −1.639 | −53.884 | 95.730 | 1.00 | 25.63 | A | C |
| ATOM | 421 | CB | VAL | 154 | −0.608 | −54.044 | 96.884 | 1.00 | 26.14 | A | C |
| ATOM | 422 | CG1 | VAL | 154 | −0.737 | −53.454 | 96.484 | 1.00 | 26.07 | A | C |
| ATOM | 423 | CG2 | VAL | 154 | −1.129 | −53.391 | 98.148 | 1.00 | 27.60 | A | C |
| ATOM | 424 | C | VAL | 154 | −1.644 | −52.432 | 95.245 | 1.00 | 25.24 | A | C |
| ATOM | 425 | O | VAL | 154 | −2.049 | −51.531 | 95.980 | 1.00 | 25.07 | A | O |
| ATOM | 426 | N | LEU | 155 | −1.200 | −52.214 | 94.010 | 1.00 | 24.31 | A | N |
| ATOM | 427 | CA | LEU | 155 | −1.150 | −50.875 | 93.418 | 1.00 | 24.23 | A | C |
| ATOM | 428 | CB | LEU | 155 | −0.863 | −50.977 | 91.910 | 1.00 | 24.21 | A | C |
| ATOM | 429 | CG | LEU | 155 | −0.776 | −49.663 | 91.118 | 1.00 | 25.59 | A | C |
| ATOM | 430 | CD1 | LEU | 155 | 0.261 | −48.739 | 91.744 | 1.00 | 24.34 | A | C |
| ATOM | 431 | CD2 | LEU | 155 | −0.426 | −49.962 | 89.658 | 1.00 | 24.80 | A | C |
| ATOM | 432 | C | LEU | 155 | −2.418 | −50.038 | 93.661 | 1.00 | 23.18 | A | C |
| ATOM | 433 | O | LEU | 155 | −2.335 | −48.915 | 94.149 | 1.00 | 23.96 | A | O |
| ATOM | 434 | N | PRO | 156 | −3.607 | −50.569 | 93.331 | 1.00 | 22.69 | A | N |
| ATOM | 435 | CD | PRO | 156 | −3.958 | −51.848 | 92.684 | 1.00 | 22.19 | A | C |
| ATOM | 436 | CA | PRO | 156 | −4.808 | −49.757 | 93.571 | 1.00 | 22.08 | A | C |
| ATOM | 437 | CB | PRO | 156 | −5.944 | −50.698 | 93.171 | 1.00 | 22.24 | A | C |
| ATOM | 438 | CG | PRO | 156 | −5.318 | −51.539 | 92.086 | 1.00 | 22.14 | A | C |
| ATOM | 439 | C | PRO | 156 | −4.928 | −49.277 | 95.031 | 1.00 | 22.20 | A | C |
| ATOM | 440 | O | PRO | 156 | −5.291 | −48.122 | 95.287 | 1.00 | 20.87 | A | O |
| ATOM | 441 | N | LEU | 157 | −4.627 | −50.164 | 95.980 | 1.00 | 20.93 | A | N |
| ATOM | 442 | CA | LEU | 157 | −4.689 | −49.813 | 97.397 | 1.00 | 20.80 | A | C |
| ATOM | 443 | CB | LEU | 157 | −4.433 | −51.046 | 98.269 | 1.00 | 19.44 | A | C |
| ATOM | 444 | CG | LEU | 157 | −4.414 | −50.793 | 99.783 | 1.00 | 19.55 | A | C |
| ATOM | 445 | CD1 | LEU | 157 | −5.738 | −50.152 | 100.220 | 1.00 | 16.57 | A | C |
| ATOM | 446 | CD2 | LEU | 157 | −4.163 | −52.113 | 100.522 | 1.00 | 16.20 | A | C |
| ATOM | 447 | C | LEU | 157 | −3.657 | −48.738 | 97.715 | 1.00 | 20.74 | A | C |
| ATOM | 448 | O | LEU | 157 | −3.950 | −47.774 | 98.422 | 1.00 | 20.79 | A | O |
| ATOM | 449 | N | VAL | 158 | −2.450 | −48.911 | 97.186 | 1.00 | 20.78 | A | N |
| ATOM | 450 | CA | VAL | 158 | −1.354 | −47.962 | 97.387 | 1.00 | 20.77 | A | C |
| ATOM | 451 | CB | VAL | 158 | −0.061 | −48.480 | 96.704 | 1.00 | 21.54 | A | C |
| ATOM | 452 | CG1 | VAL | 158 | 1.014 | −47.402 | 96.720 | 1.00 | 21.06 | A | C |
| ATOM | 453 | CG2 | VAL | 158 | 0.438 | −49.743 | 97.420 | 1.00 | 20.78 | A | C |
| ATOM | 454 | C | VAL | 158 | −1.699 | −46.583 | 96.810 | 1.00 | 21.47 | A | C |
| ATOM | 455 | O | VAL | 158 | −1.434 | −45.549 | 97.428 | 1.00 | 21.74 | A | O |
| ATOM | 456 | N | THR | 159 | −2.293 | −46.575 | 95.621 | 1.00 | 21.17 | A | N |
| ATOM | 457 | CA | THR | 159 | −2.669 | −45.334 | 94.956 | 1.00 | 21.04 | A | C |
| ATOM | 458 | CB | THR | 159 | −3.037 | −45.606 | 93.484 | 1.00 | 21.88 | A | C |
| ATOM | 459 | OG1 | THR | 159 | −2.012 | −46.413 | 92.882 | 1.00 | 22.76 | A | O |
| ATOM | 460 | CG2 | THR | 159 | −3.143 | −44.308 | 92.710 | 1.00 | 21.05 | A | C |
| ATOM | 461 | C | THR | 159 | −3.841 | −44.677 | 95.682 | 1.00 | 20.47 | A | C |
| ATOM | 462 | O | THR | 159 | −3.944 | −43.449 | 95.721 | 1.00 | 20.97 | A | O |
| ATOM | 463 | N | HIS | 160 | −4.721 | −45.500 | 96.252 | 1.00 | 19.61 | A | N |
| ATOM | 464 | CA | HIS | 160 | −5.866 | −45.009 | 97.008 | 1.00 | 20.01 | A | C |
| ATOM | 465 | CB | HIS | 160 | −6.789 | −46.161 | 97.423 | 1.00 | 20.08 | A | C |
| ATOM | 466 | CG | HIS | 160 | −7.887 | −45.744 | 98.354 | 1.00 | 20.94 | A | C |
| ATOM | 467 | CD2 | HIS | 160 | −8.085 | −45.999 | 99.669 | 1.00 | 20.95 | A | C |
| ATOM | 468 | ND1 | HIS | 160 | −8.925 | −44.925 | 97.965 | 1.00 | 21.19 | A | N |
| ATOM | 469 | CE1 | HIS | 160 | −9.713 | −44.691 | 99.000 | 1.00 | 20.93 | A | C |
| ATOM | 470 | NE2 | HIS | 160 | −9.225 | −45.331 | 100.047 | 1.00 | 20.20 | A | N |
| ATOM | 471 | C | HIS | 160 | −5.328 | −44.308 | 98.254 | 1.00 | 19.95 | A | C |
| ATOM | 472 | O | HIS | 160 | −5.784 | −43.219 | 98.600 | 1.00 | 20.42 | A | O |
| ATOM | 473 | N | PHE | 161 | −4.355 | −44.933 | 98.920 | 1.00 | 18.59 | A | N |
| ATOM | 474 | CA | PHE | 161 | −3.736 | −44.345 | 100.105 | 1.00 | 18.56 | A | C |
| ATOM | 475 | CB | PHE | 161 | −2.737 | −45.327 | 100.735 | 1.00 | 18.23 | A | C |
| ATOM | 476 | CG | PHE | 161 | −3.368 | −46.356 | 101.640 | 1.00 | 17.32 | A | C |
| ATOM | 477 | CD1 | PHE | 161 | −4.720 | −46.314 | 101.941 | 1.00 | 16.89 | A | C |
| ATOM | 478 | CD2 | PHE | 161 | −2.594 | −47.362 | 102.206 | 1.00 | 18.24 | A | C |
| ATOM | 479 | CE1 | PHE | 161 | −5.294 | −47.255 | 102.792 | 1.00 | 17.84 | A | C |
| ATOM | 480 | CE2 | PHE | 161 | −3.160 | −48.308 | 103.058 | 1.00 | 17.78 | A | C |
| ATOM | 481 | CZ | PHE | 161 | −4.517 | −48.253 | 103.352 | 1.00 | 16.70 | A | C |
| ATOM | 482 | C | PHE | 161 | −3.018 | −43.030 | 99.758 | 1.00 | 19.43 | A | C |
| ATOM | 483 | O | PHE | 161 | −3.122 | −42.041 | 100.488 | 1.00 | 18.14 | A | O |
| ATOM | 484 | N | ALA | 162 | −2.281 | −43.019 | 98.651 | 1.00 | 19.79 | A | N |
| ATOM | 485 | CA | ALA | 162 | −1.580 | −41.809 | 98.233 | 1.00 | 21.41 | A | C |
| ATOM | 486 | CB | ALA | 162 | −0.818 | −42.061 | 96.923 | 1.00 | 20.88 | A | C |
| ATOM | 487 | C | ALA | 162 | −2.598 | −40.674 | 98.040 | 1.00 | 22.02 | A | C |
| ATOM | 488 | O | ALA | 162 | −2.373 | −39.545 | 98.471 | 1.00 | 22.29 | A | O |
| ATOM | 489 | N | ASP | 163 | −3.718 | −40.991 | 97.399 | 1.00 | 22.30 | A | N |
| ATOM | 490 | CA | ASP | 163 | −4.766 | −40.012 | 97.144 | 1.00 | 22.87 | A | C |
| ATOM | 491 | CB | ASP | 163 | −5.841 | −40.626 | 96.236 | 1.00 | 23.81 | A | C |
| ATOM | 492 | CG | ASP | 163 | −5.360 | −40.804 | 94.788 | 1.00 | 25.70 | A | C |
| ATOM | 493 | OD1 | ASP | 163 | −5.966 | −41.605 | 94.044 | 1.00 | 24.91 | A | O |
| ATOM | 494 | OD2 | ASP | 163 | −4.380 | −40.134 | 94.390 | 1.00 | 25.54 | A | O |
| ATOM | 495 | C | ASP | 163 | −5.399 | −39.468 | 98.426 | 1.00 | 23.27 | A | C |
| ATOM | 496 | O | ASP | 163 | −5.426 | −38.254 | 98.633 | 1.00 | 22.56 | A | O |
| ATOM | 497 | N | ILE | 164 | −5.896 | −40.346 | 99.300 | 1.00 | 22.58 | A | N |
| ATOM | 498 | CA | ILE | 164 | −6.515 | −39.858 | 100.527 | 1.00 | 22.00 | A | C |
| ATOM | 499 | CB | ILE | 164 | −7.329 | −40.966 | 101.261 | 1.00 | 21.65 | A | C |
| ATOM | 500 | CG2 | ILE | 164 | −8.363 | −41.551 | 100.302 | 1.00 | 19.34 | A | C |
| ATOM | 501 | CG1 | ILE | 164 | −6.409 | −42.064 | 101.807 | 1.00 | 20.52 | A | C |
| ATOM | 502 | CD1 | ILE | 164 | −7.172 | −43.169 | 102.530 | 1.00 | 18.21 | A | C |
| ATOM | 503 | C | ILE | 164 | −5.524 | −39.207 | 101.490 | 1.00 | 22.19 | A | C |
| ATOM | 504 | O | ILE | 164 | −5.913 | −38.346 | 102.281 | 1.00 | 21.74 | A | O |
| ATOM | 505 | N | ASN | 165 | −4.252 | −39.599 | 101.425 | 1.00 | 21.86 | A | N |
| ATOM | 506 | CA | ASN | 165 | −3.241 | −38.988 | 102.289 | 1.00 | 22.10 | A | C |
| ATOM | 507 | CB | ASN | 165 | −1.899 | −39.729 | 102.200 | 1.00 | 20.87 | A | C |
| ATOM | 508 | CG | ASN | 165 | −1.897 | −41.047 | 102.962 | 1.00 | 21.09 | A | C |
| ATOM | 509 | OD1 | ASN | 165 | −2.778 | −41.308 | 103.784 | 1.00 | 19.71 | A | O |
| ATOM | 510 | ND2 | ASN | 165 | −0.890 | −41.880 | 102.703 | 1.00 | 19.72 | A | N |
| ATOM | 511 | C | ASN | 165 | −3.045 | −37.534 | 101.858 | 1.00 | 22.73 | A | C |
| ATOM | 512 | O | ASN | 165 | −2.989 | −36.628 | 102.690 | 1.00 | 22.46 | A | O |
| ATOM | 513 | N | THR | 166 | −2.932 | −37.327 | 100.549 | 1.00 | 23.63 | A | N |
| ATOM | 514 | CA | THR | 166 | −2.758 | −35.994 | 99.975 | 1.00 | 24.53 | A | C |
| ATOM | 515 | CB | THR | 166 | −2.564 | −36.082 | 98.448 | 1.00 | 25.05 | A | C |
| ATOM | 516 | OG1 | THR | 166 | −1.374 | −36.830 | 98.166 | 1.00 | 25.95 | A | O |
| ATOM | 517 | CG2 | THR | 166 | −2.451 | −34.689 | 97.834 | 1.00 | 24.40 | A | C |
| ATOM | 518 | C | THR | 166 | −4.002 | −35.168 | 100.281 | 1.00 | 24.71 | A | C |
| ATOM | 519 | O | THR | 166 | −3.911 | −34.020 | 100.727 | 1.00 | 25.33 | A | O |
| ATOM | 520 | N | PHE | 167 | −5.165 | −35.765 | 100.034 | 1.00 | 23.87 | A | N |
| ATOM | 521 | CA | PHE | 167 | −6.438 | −35.125 | 100.310 | 1.00 | 23.69 | A | C |
| ATOM | 522 | CB | PHE | 167 | −7.576 | −36.132 | 100.111 | 1.00 | 23.28 | A | C |
| ATOM | 523 | CG | PHE | 167 | −8.884 | −35.699 | 100.719 | 1.00 | 23.25 | A | C |
| ATOM | 524 | CD1 | PHE | 167 | −9.600 | −34.629 | 100.184 | 1.00 | 23.28 | A | C |
| ATOM | 525 | CD2 | PHE | 167 | −9.390 | −36.351 | 101.842 | 1.00 | 22.82 | A | C |
| ATOM | 526 | CE1 | PHE | 167 | −10.800 | −34.213 | 100.757 | 1.00 | 21.97 | A | C |
| ATOM | 527 | CE2 | PHE | 167 | −10.589 | −35.946 | 102.427 | 1.00 | 22.68 | A | C |
| ATOM | 528 | CZ | PHE | 167 | −11.298 | −34.872 | 101.880 | 1.00 | 22.73 | A | C |
| ATOM | 529 | C | PHE | 167 | −6.440 | −34.611 | 101.755 | 1.00 | 24.07 | A | C |
| ATOM | 530 | O | PHE | 167 | −6.676 | −33.427 | 101.996 | 1.00 | 24.74 | A | O |
| ATOM | 531 | N | MET | 168 | −6.161 | −35.508 | 102.702 | 1.00 | 22.88 | A | N |
| ATOM | 532 | CA | MET | 168 | −6.133 | −35.168 | 104.123 | 1.00 | 22.54 | A | C |
| ATOM | 533 | CB | MET | 168 | −5.844 | −36.420 | 104.974 | 1.00 | 20.50 | A | C |
| ATOM | 534 | CG | MET | 168 | −7.020 | −37.378 | 105.090 | 1.00 | 19.43 | A | C |
| ATOM | 535 | SD | MET | 168 | −6.792 | −38.627 | 106.404 | 1.00 | 16.80 | A | S |
| ATOM | 536 | CE | MET | 168 | −5.899 | −39.880 | 105.499 | 1.00 | 19.32 | A | C |
| ATOM | 537 | C | MET | 168 | −5.137 | −34.065 | 104.472 | 1.00 | 22.25 | A | C |
| ATOM | 538 | O | MET | 168 | −5.459 | −33.160 | 105.226 | 1.00 | 21.58 | A | O |
| ATOM | 539 | N | VAL | 169 | −3.928 | −34.144 | 103.932 | 1.00 | 22.92 | A | N |
| ATOM | 540 | CA | VAL | 169 | −2.927 | −33.112 | 104.186 | 1.00 | 23.69 | A | C |
| ATOM | 541 | CB | VAL | 169 | −1.635 | −33.375 | 103.383 | 1.00 | 24.08 | A | C |
| ATOM | 542 | CG1 | VAL | 169 | −0.668 | −32.208 | 103.555 | 1.00 | 25.04 | A | C |
| ATOM | 543 | CG2 | VAL | 169 | −0.981 | −34.661 | 103.863 | 1.00 | 24.66 | A | C |
| ATOM | 544 | C | VAL | 169 | −3.470 | −31.733 | 103.794 | 1.00 | 24.06 | A | C |
| ATOM | 545 | O | VAL | 169 | −3.335 | −30.759 | 104.546 | 1.00 | 23.08 | A | O |
| ATOM | 546 | N | LEU | 170 | −4.084 | −31.656 | 102.616 | 1.00 | 24.09 | A | N |
| ATOM | 547 | CA | LEU | 170 | −4.650 | −30.404 | 102.131 | 1.00 | 24.47 | A | C |
| ATOM | 548 | CB | LEU | 170 | −5.170 | −30.587 | 100.702 | 1.00 | 25.76 | A | C |
| ATOM | 549 | CG | LEU | 170 | −4.109 | −30.969 | 99.660 | 1.00 | 27.02 | A | C |
| ATOM | 550 | CD1 | LEU | 170 | −4.776 | −31.173 | 98.303 | 1.00 | 27.49 | A | C |
| ATOM | 551 | CD2 | LEU | 170 | −3.037 | −29.881 | 99.575 | 1.00 | 27.84 | A | C |
| ATOM | 552 | C | LEU | 170 | −5.772 | −29.919 | 103.054 | 1.00 | 24.53 | A | C |
| ATOM | 553 | O | LEU | 170 | −5.954 | −28.715 | 103.246 | 1.00 | 24.40 | A | O |
| ATOM | 554 | N | GLN | 171 | −6.519 | −30.855 | 103.637 | 1.00 | 23.66 | A | N |
| ATOM | 555 | CA | GLN | 171 | −7.589 | −30.484 | 104.550 | 1.00 | 23.20 | A | C |
| ATOM | 556 | CB | GLN | 171 | −8.482 | −31.692 | 104.856 | 1.00 | 22.89 | A | C |
| ATOM | 557 | CG | GLN | 171 | −9.301 | −32.169 | 103.657 | 1.00 | 22.15 | A | C |
| ATOM | 558 | CD | GLN | 171 | −10.227 | −31.085 | 103.108 | 1.00 | 21.39 | A | C |
| ATOM | 559 | OE1 | GLN | 171 | −11.164 | −30.644 | 103.777 | 1.00 | 20.79 | A | O |
| ATOM | 560 | NE2 | GLN | 171 | −9.960 | −30.650 | 101.888 | 1.00 | 20.38 | A | N |
| ATOM | 561 | C | GLN | 171 | −7.004 | −29.920 | 105.841 | 1.00 | 23.71 | A | C |
| ATOM | 562 | O | GLN | 171 | −7.621 | −29.071 | 106.485 | 1.00 | 22.77 | A | O |
| ATOM | 563 | N | VAL | 172 | −5.820 | −30.390 | 106.232 | 1.00 | 24.17 | A | N |
| ATOM | 564 | CA | VAL | 172 | −5.195 | −29.868 | 107.445 | 1.00 | 25.12 | A | C |
| ATOM | 565 | CB | VAL | 172 | −3.989 | −30.714 | 107.893 | 1.00 | 25.29 | A | C |
| ATOM | 566 | CG1 | VAL | 172 | −3.268 | −30.005 | 109.034 | 1.00 | 23.34 | A | C |
| ATOM | 567 | CG2 | VAL | 172 | −4.450 | −32.098 | 108.339 | 1.00 | 24.47 | A | C |
| ATOM | 568 | C | VAL | 172 | −4.711 | −28.440 | 107.193 | 1.00 | 25.52 | A | C |
| ATOM | 569 | O | VAL | 172 | −4.833 | −27.565 | 108.057 | 1.00 | 25.32 | A | O |
| ATOM | 570 | N | ILE | 173 | −4.149 | −28.215 | 106.010 | 1.00 | 26.12 | A | N |
| ATOM | 571 | CA | ILE | 173 | −3.663 | −26.895 | 105.646 | 1.00 | 26.88 | A | C |
| ATOM | 572 | CB | ILE | 173 | −3.062 | −26.902 | 104.231 | 1.00 | 26.76 | A | C |
| ATOM | 573 | CG2 | ILE | 173 | −2.686 | −25.491 | 103.822 | 1.00 | 26.79 | A | C |
| ATOM | 574 | CG1 | ILE | 173 | −1.835 | −27.822 | 104.208 | 1.00 | 27.16 | A | C |
| ATOM | 575 | CD1 | ILE | 173 | −1.135 | −27.937 | 102.866 | 1.00 | 27.08 | A | C |
| ATOM | 576 | C | ILE | 173 | −4.837 | −25.919 | 105.718 | 1.00 | 27.47 | A | C |
| ATOM | 577 | O | ILE | 173 | −4.729 | −24.847 | 106.320 | 1.00 | 27.14 | A | O |
| ATOM | 578 | N | LYS | 174 | −5.962 | −26.305 | 105.122 | 1.00 | 27.73 | A | N |
| ATOM | 579 | CA | LYS | 174 | −7.162 | −25.473 | 105.141 | 1.00 | 28.31 | A | C |
| ATOM | 580 | CB | LYS | 174 | −8.264 | −26.123 | 104.296 | 1.00 | 28.44 | A | C |
| ATOM | 581 | CG | LYS | 174 | −7.935 | −26.108 | 102.803 | 1.00 | 29.80 | A | C |
| ATOM | 582 | CD | LYS | 174 | −8.878 | −26.964 | 101.975 | 1.00 | 31.27 | A | C |
| ATOM | 583 | CE | LYS | 174 | −10.279 | −26.412 | 101.958 | 1.00 | 32.16 | A | C |
| ATOM | 584 | NZ | LYS | 174 | −11.147 | −27.278 | 101.116 | 1.00 | 34.46 | A | N |
| ATOM | 585 | C | LYS | 174 | −7.647 | −25.232 | 106.570 | 1.00 | 28.11 | A | C |
| ATOM | 586 | O | LYS | 174 | −8.142 | −24.147 | 106.885 | 1.00 | 28.60 | A | O |
| ATOM | 587 | N | PHE | 175 | −7.499 | −26.240 | 107.429 | 1.00 | 27.63 | A | N |
| ATOM | 588 | CA | PHE | 175 | −7.898 | −26.142 | 108.834 | 1.00 | 27.68 | A | C |
| ATOM | 589 | CB | PHE | 175 | −7.736 | −27.507 | 109.524 | 1.00 | 27.35 | A | C |
| ATOM | 590 | CG | PHE | 175 | −7.842 | −27.452 | 111.029 | 1.00 | 27.54 | A | C |
| ATOM | 591 | CD1 | PHE | 175 | −9.041 | −27.119 | 111.651 | 1.00 | 27.14 | A | C |
| ATOM | 592 | CD2 | PHE | 175 | −6.730 | −27.709 | 111.822 | 1.00 | 27.58 | A | C |
| ATOM | 593 | CE1 | PHE | 175 | −9.129 | −27.040 | 113.039 | 1.00 | 26.74 | A | C |
| ATOM | 594 | CE2 | PHE | 175 | −6.806 | −27.632 | 113.215 | 1.00 | 27.74 | A | C |
| ATOM | 595 | CZ | PHE | 175 | −8.010 | −27.296 | 113.823 | 1.00 | 27.47 | A | C |
| ATOM | 596 | C | PHE | 175 | −7.042 | −25.094 | 109.551 | 1.00 | 28.52 | A | C |
| ATOM | 597 | O | PHE | 175 | −7.558 | −24.244 | 110.268 | 1.00 | 27.62 | A | O |
| ATOM | 598 | N | THR | 176 | −5.731 | −25.148 | 109.343 | 1.00 | 29.79 | A | N |
| ATOM | 599 | CA | THR | 176 | −4.824 | −24.204 | 109.985 | 1.00 | 31.23 | A | C |
| ATOM | 600 | CB | THR | 176 | −3.358 | −24.621 | 109.789 | 1.00 | 30.67 | A | C |
| ATOM | 601 | OG1 | THR | 176 | −3.022 | −24.563 | 108.399 | 1.00 | 30.30 | A | O |
| ATOM | 602 | CG2 | THR | 176 | −3.138 | −26.036 | 110.306 | 1.00 | 31.22 | A | C |
| ATOM | 603 | C | THR | 176 | −4.998 | −22.774 | 109.479 | 1.00 | 32.33 | A | C |
| ATOM | 604 | O | THR | 176 | −4.812 | −21.819 | 110.232 | 1.00 | 32.61 | A | O |
| ATOM | 605 | N | LYS | 177 | −5.358 | −22.633 | 108.207 | 1.00 | 33.30 | A | N |
| ATOM | 606 | CA | LYS | 177 | −5.565 | −21.319 | 107.602 | 1.00 | 34.34 | A | C |
| ATOM | 607 | CB | LYS | 177 | −5.823 | −21.465 | 106.105 | 1.00 | 35.54 | A | C |
| ATOM | 608 | CG | LYS | 177 | −4.625 | −21.950 | 105.310 | 1.00 | 38.15 | A | C |
| ATOM | 609 | CD | LYS | 177 | −3.612 | −20.844 | 105.087 | 1.00 | 39.75 | A | C |
| ATOM | 610 | CE | LYS | 177 | −2.354 | −21.393 | 104.434 | 1.00 | 40.78 | A | C |
| ATOM | 611 | NZ | LYS | 177 | −2.655 | −22.148 | 103.187 | 1.00 | 41.40 | A | N |
| ATOM | 612 | C | LYS | 177 | −6.732 | −20.572 | 108.243 | 1.00 | 34.59 | A | C |
| ATOM | 613 | O | LYS | 177 | −6.806 | −19.345 | 108.153 | 1.00 | 35.04 | A | O |
| ATOM | 614 | N | ASP | 178 | −7.642 | −21.314 | 108.874 | 1.00 | 34.14 | A | N |
| ATOM | 615 | CA | ASP | 178 | −8.802 | −20.730 | 109.543 | 1.00 | 34.02 | A | C |
| ATOM | 616 | CB | ASP | 178 | −10.006 | −21.668 | 109.445 | 1.00 | 34.24 | A | C |
| ATOM | 617 | CG | ASP | 178 | −10.744 | −21.539 | 108.129 | 1.00 | 34.92 | A | C |
| ATOM | 618 | OD1 | ASP | 178 | −10.332 | −20.726 | 107.277 | 1.00 | 35.28 | A | O |
| ATOM | 619 | OD2 | ASP | 178 | −11.749 | −22.252 | 107.950 | 1.00 | 36.67 | A | O |
| ATOM | 620 | C | ASP | 178 | −8.542 | −20.419 | 111.013 | 1.00 | 33.84 | A | C |
| ATOM | 621 | O | ASP | 178 | −9.468 | −20.085 | 111.752 | 1.00 | 34.10 | A | O |
| ATOM | 622 | N | LEU | 179 | −7.288 | −20.542 | 111.439 | 1.00 | 33.64 | A | N |
| ATOM | 623 | CA | LEU | 179 | −6.916 | −20.262 | 112.819 | 1.00 | 33.48 | A | C |
| ATOM | 624 | CB | LEU | 179 | −6.100 | −21.420 | 113.398 | 1.00 | 32.20 | A | C |
| ATOM | 625 | CG | LEU | 179 | −6.695 | −22.827 | 113.240 | 1.00 | 31.98 | A | C |
| ATOM | 626 | CD1 | LEU | 179 | −5.809 | −23.831 | 113.963 | 1.00 | 31.56 | A | C |
| ATOM | 627 | CD2 | LEU | 179 | −8.107 | −22.876 | 113.793 | 1.00 | 30.73 | A | C |
| ATOM | 628 | C | LEU | 179 | −6.090 | −18.981 | 112.834 | 1.00 | 33.94 | A | C |
| ATOM | 629 | O | LEU | 179 | −4.913 | −18.982 | 112.464 | 1.00 | 33.94 | A | O |
| ATOM | 630 | N | PRO | 180 | −6.703 | −17.863 | 113.252 | 1.00 | 34.34 | A | N |
| ATOM | 631 | CD | PRO | 180 | −8.097 | −17.743 | 113.718 | 1.00 | 34.10 | A | C |
| ATOM | 632 | CA | PRO | 180 | −6.028 | −16.564 | 113.312 | 1.00 | 34.65 | A | C |
| ATOM | 633 | CB | PRO | 180 | −6.994 | −15.725 | 114.135 | 1.00 | 34.81 | A | C |
| ATOM | 634 | CG | PRO | 180 | −8.322 | −16.250 | 113.675 | 1.00 | 34.48 | A | C |
| ATOM | 635 | C | PRO | 180 | −4.631 | −16.606 | 113.916 | 1.00 | 35.01 | A | C |
| ATOM | 636 | O | PRO | 180 | −3.680 | −16.112 | 113.320 | 1.00 | 34.47 | A | O |
| ATOM | 637 | N | VAL | 181 | −4.501 | −17.208 | 115.092 | 1.00 | 35.81 | A | N |
| ATOM | 638 | CA | VAL | 181 | −3.200 | −17.275 | 115.745 | 1.00 | 36.75 | A | C |
| ATOM | 639 | CB | VAL | 181 | −3.311 | −17.889 | 117.149 | 1.00 | 37.21 | A | C |
| ATOM | 640 | CG1 | VAL | 181 | −1.931 | −18.014 | 117.762 | 1.00 | 38.68 | A | C |
| ATOM | 641 | CG2 | VAL | 181 | −4.189 | −17.007 | 118.032 | 1.00 | 38.14 | A | C |
| ATOM | 642 | C | VAL | 181 | −2.165 | −18.051 | 114.935 | 1.00 | 36.94 | A | C |
| ATOM | 643 | O | VAL | 181 | −0.978 | −17.730 | 114.976 | 1.00 | 36.68 | A | O |
| ATOM | 644 | N | PHE | 182 | −2.604 | −19.069 | 114.198 | 1.00 | 37.05 | A | N |
| ATOM | 645 | CA | PHE | 182 | −1.677 | −19.847 | 113.378 | 1.00 | 37.75 | A | C |
| ATOM | 646 | CB | PHE | 182 | −2.325 | −21.141 | 112.878 | 1.00 | 37.39 | A | C |
| ATOM | 647 | CG | PHE | 182 | −1.431 | −21.945 | 111.972 | 1.00 | 37.39 | A | C |
| ATOM | 648 | CD1 | PHE | 182 | −0.475 | −22.806 | 112.500 | 1.00 | 37.05 | A | C |
| ATOM | 649 | CD2 | PHE | 182 | −1.513 | −21.805 | 110.590 | 1.00 | 37.20 | A | C |
| ATOM | 650 | CE1 | PHE | 182 | 0.387 | −23.517 | 111.663 | 1.00 | 36.56 | A | C |
| ATOM | 651 | CE2 | PHE | 182 | −0.653 | −22.511 | 109.746 | 1.00 | 37.00 | A | C |
| ATOM | 652 | CZ | PHE | 182 | 0.297 | −23.368 | 110.286 | 1.00 | 36.96 | A | C |
| ATOM | 653 | C | PHE | 182 | −1.242 | −19.036 | 112.163 | 1.00 | 38.08 | A | C |
| ATOM | 654 | O | PHE | 182 | −0.063 | −18.976 | 111.826 | 1.00 | 37.90 | A | O |
| ATOM | 655 | N | ARG | 183 | −2.214 | −18.426 | 111.499 | 1.00 | 39.10 | A | N |
| ATOM | 656 | CA | ARG | 183 | −1.948 | −17.630 | 110.314 | 1.00 | 40.37 | A | C |
| ATOM | 657 | CB | ARG | 183 | −3.268 | −17.242 | 109.661 | 1.00 | 40.60 | A | C |
| ATOM | 658 | CG | ARG | 183 | −3.094 | −16.612 | 108.314 | 1.00 | 41.01 | A | C |
| ATOM | 659 | CD | ARG | 183 | −4.196 | −17.041 | 107.394 | 1.00 | 40.77 | A | C |
| ATOM | 660 | NE | ARG | 183 | −4.070 | −16.387 | 106.101 | 1.00 | 40.69 | A | N |
| ATOM | 661 | CZ | ARG | 183 | −5.001 | −16.419 | 105.160 | 1.00 | 39.62 | A | C |
| ATOM | 662 | NH1 | ARG | 183 | −6.132 | −17.079 | 105.370 | 1.00 | 39.80 | A | N |
| ATOM | 663 | NH2 | ARG | 183 | −4.800 | −15.783 | 104.017 | 1.00 | 38.99 | A | N |
| ATOM | 664 | C | ARG | 183 | −1.129 | −16.381 | 110.618 | 1.00 | 41.43 | A | C |
| ATOM | 665 | O | ARG | 183 | −0.433 | −15.856 | 109.747 | 1.00 | 41.39 | A | O |
| ATOM | 666 | N | SER | 184 | −1.215 | −15.910 | 111.858 | 1.00 | 42.46 | A | N |
| ATOM | 667 | CA | SER | 184 | −0.484 | −14.728 | 112.292 | 1.00 | 43.82 | A | C |
| ATOM | 668 | CB | SER | 184 | −1.051 | −14.236 | 113.620 | 1.00 | 44.39 | A | C |
| ATOM | 669 | OG | SER | 184 | −0.499 | −12.984 | 113.959 | 1.00 | 47.04 | A | O |
| ATOM | 670 | C | SER | 184 | 1.016 | −14.996 | 112.439 | 1.00 | 44.32 | A | C |
| ATOM | 671 | O | SER | 184 | 1.813 | −14.060 | 112.538 | 1.00 | 44.62 | A | O |
| ATOM | 672 | N | LEU | 185 | 1.395 | −16.273 | 112.459 | 1.00 | 44.27 | A | N |
| ATOM | 673 | CA | LEU | 185 | 2.796 | −16.666 | 112.578 | 1.00 | 44.64 | A | C |
| ATOM | 674 | CB | LEU | 185 | 2.903 | −18.150 | 112.945 | 1.00 | 43.86 | A | C |
| ATOM | 675 | CG | LEU | 185 | 2.344 | −18.644 | 114.282 | 1.00 | 44.09 | A | C |
| ATOM | 676 | CD1 | LEU | 185 | 2.459 | −20.166 | 114.362 | 1.00 | 42.72 | A | C |
| ATOM | 677 | CD2 | LEU | 185 | 3.103 | −17.992 | 115.423 | 1.00 | 43.21 | A | C |
| ATOM | 678 | C | LEU | 185 | 3.510 | −16.447 | 111.248 | 1.00 | 45.42 | A | C |
| ATOM | 679 | O | LEU | 185 | 2.876 | −16.405 | 110.194 | 1.00 | 45.54 | A | O |
| ATOM | 680 | N | PRO | 186 | 4.844 | −16.304 | 111.278 | 1.00 | 46.26 | A | N |
| ATOM | 681 | CD | PRO | 186 | 5.775 | −16.356 | 112.418 | 1.00 | 46.30 | A | C |
| ATOM | 682 | CA | PRO | 186 | 5.566 | −16.103 | 110.018 | 1.00 | 46.87 | A | C |
| ATOM | 683 | CB | PRO | 186 | 7.008 | −15.893 | 110.477 | 1.00 | 46.47 | A | C |
| ATOM | 684 | CG | PRO | 186 | 7.084 | −16.693 | 111.741 | 1.00 | 47.00 | A | C |
| ATOM | 685 | C | PRO | 186 | 5.390 | −17.348 | 109.144 | 1.00 | 47.62 | A | C |
| ATOM | 686 | O | PRO | 186 | 5.308 | −18.463 | 109.659 | 1.00 | 48.13 | A | O |
| ATOM | 687 | N | ILE | 187 | 5.328 | −17.154 | 107.830 | 1.00 | 47.94 | A | N |
| ATOM | 688 | CA | ILE | 187 | 5.131 | −18.256 | 106.891 | 1.00 | 47.82 | A | C |
| ATOM | 689 | CB | ILE | 187 | 5.236 | −17.749 | 105.423 | 1.00 | 48.13 | A | C |
| ATOM | 690 | CG2 | ILE | 187 | 6.601 | −17.105 | 105.182 | 1.00 | 48.93 | A | C |
| ATOM | 691 | CG1 | ILE | 187 | 4.975 | −18.895 | 104.442 | 1.00 | 48.18 | A | C |
| ATOM | 692 | CD1 | ILE | 187 | 6.169 | −19.808 | 104.180 | 1.00 | 48.34 | A | C |
| ATOM | 693 | C | ILE | 187 | 6.055 | −19.460 | 107.095 | 1.00 | 47.81 | A | C |
| ATOM | 694 | O | ILE | 187 | 5.614 | −20.602 | 106.967 | 1.00 | 47.87 | A | O |
| ATOM | 695 | N | GLU | 188 | 7.327 | −19.221 | 107.405 | 1.00 | 47.20 | A | N |
| ATOM | 696 | CA | GLU | 188 | 8.265 | −20.320 | 107.619 | 1.00 | 46.46 | A | C |
| ATOM | 697 | CB | GLU | 188 | 9.702 | −19.800 | 107.799 | 1.00 | 47.64 | A | C |
| ATOM | 698 | CG | GLU | 188 | 9.846 | −18.469 | 108.533 | 1.00 | 50.01 | A | C |
| ATOM | 699 | CD | GLU | 188 | 9.421 | −17.284 | 107.682 | 1.00 | 51.33 | A | C |
| ATOM | 700 | OE1 | GLU | 188 | 10.033 | −17.060 | 106.614 | 1.00 | 51.74 | A | O |
| ATOM | 701 | OE2 | GLU | 188 | 8.465 | −16.582 | 108.081 | 1.00 | 52.98 | A | O |
| ATOM | 702 | C | GLU | 188 | 7.860 | −21.182 | 108.814 | 1.00 | 45.35 | A | C |
| ATOM | 703 | O | GLU | 188 | 7.975 | −22.407 | 108.769 | 1.00 | 44.56 | A | O |
| ATOM | 704 | N | ASP | 189 | 7.385 | −20.544 | 109.879 | 1.00 | 44.44 | A | N |
| ATOM | 705 | CA | ASP | 189 | 6.945 | −21.271 | 111.062 | 1.00 | 43.61 | A | C |
| ATOM | 706 | CB | ASP | 189 | 6.674 | −20.307 | 112.215 | 1.00 | 44.85 | A | C |
| ATOM | 707 | CG | ASP | 189 | 7.942 | −19.883 | 112.922 | 1.00 | 45.63 | A | C |
| ATOM | 708 | OD1 | ASP | 189 | 7.856 | −19.099 | 113.886 | 1.00 | 46.80 | A | O |
| ATOM | 709 | OD2 | ASP | 189 | 9.027 | −20.339 | 112.513 | 1.00 | 47.10 | A | O |
| ATOM | 710 | C | ASP | 189 | 5.689 | −22.070 | 110.750 | 1.00 | 42.60 | A | C |
| ATOM | 711 | O | ASP | 189 | 5.476 | −23.142 | 111.309 | 1.00 | 42.20 | A | O |
| ATOM | 712 | N | GLN | 190 | 4.855 | −21.541 | 109.860 | 1.00 | 41.71 | A | N |
| ATOM | 713 | CA | GLN | 190 | 3.636 | −22.230 | 109.463 | 1.00 | 41.03 | A | C |
| ATOM | 714 | CB | GLN | 190 | 2.793 | −21.346 | 108.548 | 1.00 | 40.62 | A | C |
| ATOM | 715 | CG | GLN | 190 | 2.293 | −20.072 | 109.203 | 1.00 | 40.82 | A | C |
| ATOM | 716 | CD | GLN | 190 | 1.285 | −19.330 | 108.348 | 1.00 | 40.63 | A | C |
| ATOM | 717 | OE1 | GLN | 190 | 0.993 | −18.158 | 108.590 | 1.00 | 41.15 | A | O |
| ATOM | 718 | NE2 | GLN | 190 | 0.737 | −20.012 | 107.350 | 1.00 | 39.87 | A | N |
| ATOM | 719 | C | GLN | 190 | 4.005 | −23.514 | 108.733 | 1.00 | 40.60 | A | C |
| ATOM | 720 | O | GLN | 190 | 3.408 | −24.561 | 108.964 | 1.00 | 40.55 | A | O |
| ATOM | 721 | N | ILE | 191 | 4.993 | −23.422 | 107.850 | 1.00 | 40.63 | A | N |
| ATOM | 722 | CA | ILE | 191 | 5.464 | −24.575 | 107.095 | 1.00 | 40.39 | A | C |
| ATOM | 723 | CB | ILE | 191 | 6.622 | −24.196 | 106.142 | 1.00 | 41.16 | A | C |
| ATOM | 724 | CG2 | ILE | 191 | 7.032 | −25.409 | 105.325 | 1.00 | 41.08 | A | C |
| ATOM | 725 | CG1 | ILE | 191 | 6.206 | −23.050 | 105.216 | 1.00 | 42.55 | A | C |
| ATOM | 726 | CD1 | ILE | 191 | 5.024 | −23.362 | 104.324 | 1.00 | 43.94 | A | C |
| ATOM | 727 | C | ILE | 191 | 5.982 | −25.635 | 108.061 | 1.00 | 39.72 | A | C |
| ATOM | 728 | O | ILE | 191 | 5.573 | −26.792 | 108.009 | 1.00 | 40.14 | A | O |
| ATOM | 729 | N | SER | 192 | 6.886 | −25.227 | 108.944 | 1.00 | 38.77 | A | N |
| ATOM | 730 | CA | SER | 192 | 7.476 | −26.135 | 109.918 | 1.00 | 38.58 | A | C |
| ATOM | 731 | CB | SER | 192 | 8.433 | −25.369 | 110.836 | 1.00 | 39.73 | A | C |
| ATOM | 732 | OG | SER | 192 | 9.405 | −24.673 | 110.072 | 1.00 | 41.62 | A | O |
| ATOM | 733 | C | SER | 192 | 6.425 | −26.850 | 110.760 | 1.00 | 36.86 | A | C |
| ATOM | 734 | O | SER | 192 | 6.443 | −28.075 | 110.868 | 1.00 | 36.31 | A | O |
| ATOM | 735 | N | LEU | 193 | 5.512 | −26.087 | 111.353 | 1.00 | 35.10 | A | N |
| ATOM | 736 | CA | LEU | 193 | 4.469 | −26.675 | 112.181 | 1.00 | 34.68 | A | C |
| ATOM | 737 | CB | LEU | 193 | 3.611 | −25.580 | 112.821 | 1.00 | 34.16 | A | C |
| ATOM | 738 | CG | LEU | 193 | 4.279 | −24.708 | 113.887 | 1.00 | 34.02 | A | C |
| ATOM | 739 | CD1 | LEU | 193 | 3.258 | −23.734 | 114.445 | 1.00 | 33.54 | A | C |
| ATOM | 740 | CD2 | LEU | 193 | 4.842 | −25.581 | 115.005 | 1.00 | 33.52 | A | C |
| ATOM | 741 | C | LEU | 193 | 3.582 | −27.636 | 111.395 | 1.00 | 34.25 | A | C |
| ATOM | 742 | O | LEU | 193 | 3.222 | −28.699 | 111.888 | 1.00 | 32.86 | A | O |
| ATOM | 743 | N | LEU | 194 | 3.241 | −27.257 | 110.169 | 1.00 | 34.57 | A | N |
| ATOM | 744 | CA | LEU | 194 | 2.399 | −28.084 | 109.317 | 1.00 | 35.38 | A | C |
| ATOM | 745 | CB | LEU | 194 | 2.075 | −27.335 | 108.025 | 1.00 | 36.27 | A | C |
| ATOM | 746 | CG | LEU | 194 | 0.950 | −27.911 | 107.161 | 1.00 | 37.99 | A | C |
| ATOM | 747 | CD1 | LEU | 194 | −0.388 | −27.777 | 107.890 | 1.00 | 36.63 | A | C |
| ATOM | 748 | CD2 | LEU | 194 | 0.906 | −27.162 | 105.833 | 1.00 | 39.17 | A | C |
| ATOM | 749 | C | LEU | 194 | 3.061 | −29.420 | 108.979 | 1.00 | 35.03 | A | C |
| ATOM | 750 | O | LEU | 194 | 2.418 | −30.470 | 109.027 | 1.00 | 34.17 | A | O |
| ATOM | 751 | N | LYS | 195 | 4.342 | −29.376 | 108.626 | 1.00 | 34.97 | A | N |
| ATOM | 752 | CA | LYS | 195 | 5.079 | −30.587 | 108.285 | 1.00 | 34.93 | A | C |
| ATOM | 753 | CB | LYS | 195 | 6.491 | −30.241 | 107.796 | 1.00 | 36.64 | A | C |
| ATOM | 754 | CG | LYS | 195 | 6.542 | −29.420 | 106.513 | 1.00 | 38.77 | A | C |
| ATOM | 755 | CD | LYS | 195 | 7.978 | −29.272 | 106.017 | 1.00 | 40.24 | A | C |
| ATOM | 756 | CE | LYS | 195 | 8.054 | −28.509 | 104.698 | 1.00 | 40.73 | A | C |
| ATOM | 757 | NZ | LYS | 195 | 7.263 | −29.167 | 103.615 | 1.00 | 42.42 | A | N |
| ATOM | 758 | C | LYS | 195 | 5.190 | −31.510 | 109.493 | 1.00 | 33.81 | A | C |
| ATOM | 759 | O | LYS | 195 | 5.027 | −32.729 | 109.382 | 1.00 | 33.63 | A | O |
| ATOM | 760 | N | GLY | 196 | 5.465 | −30.921 | 110.650 | 1.00 | 32.20 | A | N |
| ATOM | 761 | CA | GLY | 196 | 5.617 | −31.713 | 111.852 | 1.00 | 30.60 | A | C |
| ATOM | 762 | C | GLY | 196 | 4.346 | −32.292 | 112.443 | 1.00 | 29.37 | A | C |
| ATOM | 763 | O | GLY | 196 | 4.400 | −33.342 | 113.085 | 1.00 | 28.60 | A | O |
| ATOM | 764 | N | ALA | 197 | 3.202 | −31.648 | 112.210 | 1.00 | 27.66 | A | N |
| ATOM | 765 | CA | ALA | 197 | 1.958 | −32.119 | 112.804 | 1.00 | 26.31 | A | C |
| ATOM | 766 | CB | ALA | 197 | 1.400 | −31.028 | 113.699 | 1.00 | 26.25 | A | C |
| ATOM | 767 | C | ALA | 197 | 0.836 | −32.666 | 111.919 | 1.00 | 25.33 | A | C |
| ATOM | 768 | O | ALA | 197 | −0.058 | −33.329 | 112.427 | 1.00 | 24.11 | A | O |
| ATOM | 769 | N | ALA | 198 | 0.869 | −32.396 | 110.617 | 1.00 | 24.59 | A | N |
| ATOM | 770 | CA | ALA | 198 | −0.188 | −32.860 | 109.702 | 1.00 | 23.71 | A | C |
| ATOM | 771 | CB | ALA | 198 | 0.250 | −32.663 | 108.248 | 1.00 | 21.84 | A | C |
| ATOM | 772 | C | ALA | 198 | −0.656 | −34.307 | 109.901 | 1.00 | 22.64 | A | C |
| ATOM | 773 | O | ALA | 198 | −1.838 | −34.554 | 110.102 | 1.00 | 22.27 | A | O |
| ATOM | 774 | N | VAL | 199 | 0.267 | −35.259 | 109.825 | 1.00 | 21.45 | A | N |
| ATOM | 775 | CA | VAL | 199 | −0.081 | −36.664 | 109.988 | 1.00 | 20.82 | A | C |
| ATOM | 776 | CB | VAL | 199 | 1.132 | −37.567 | 109.682 | 1.00 | 20.86 | A | C |
| ATOM | 777 | CG1 | VAL | 199 | 0.811 | −39.020 | 110.025 | 1.00 | 20.51 | A | C |
| ATOM | 778 | CG2 | VAL | 199 | 1.496 | −37.441 | 108.201 | 1.00 | 19.73 | A | C |
| ATOM | 779 | C | VAL | 199 | −0.616 | −36.961 | 111.387 | 1.00 | 21.29 | A | C |
| ATOM | 780 | O | VAL | 199 | −1.569 | −37.724 | 111.539 | 1.00 | 20.92 | A | O |
| ATOM | 781 | N | GLU | 200 | −0.012 | −36.353 | 112.406 | 1.00 | 20.35 | A | N |
| ATOM | 782 | CA | GLU | 200 | −0.463 | −36.549 | 113.775 | 1.00 | 21.19 | A | C |
| ATOM | 783 | CB | GLU | 200 | 0.444 | −35.780 | 114.747 | 1.00 | 21.68 | A | C |
| ATOM | 784 | CG | GLU | 200 | 1.844 | −36.367 | 114.891 | 1.00 | 22.67 | A | C |
| ATOM | 785 | CD | GLU | 200 | 2.713 | −35.615 | 115.895 | 1.00 | 23.90 | A | C |
| ATOM | 786 | OE1 | GLU | 200 | 2.179 | −34.813 | 116.690 | 1.00 | 23.20 | A | O |
| ATOM | 787 | OE2 | GLU | 200 | 3.941 | −35.845 | 115.897 | 1.00 | 25.78 | A | O |
| ATOM | 788 | C | GLU | 200 | −1.919 | −36.082 | 113.930 | 1.00 | 21.10 | A | C |
| ATOM | 789 | O | GLU | 200 | −2.766 | −36.802 | 114.466 | 1.00 | 20.46 | A | O |
| ATOM | 790 | N | ILE | 201 | −2.200 | −34.872 | 113.459 | 1.00 | 20.78 | A | N |
| ATOM | 791 | CA | ILE | 201 | −3.543 | −34.310 | 113.531 | 1.00 | 21.26 | A | C |
| ATOM | 792 | CB | ILE | 201 | −3.593 | −32.896 | 112.901 | 1.00 | 21.50 | A | C |
| ATOM | 793 | CG2 | ILE | 201 | −5.042 | −32.407 | 112.825 | 1.00 | 22.57 | A | C |
| ATOM | 794 | CG1 | ILE | 201 | −2.753 | −31.919 | 113.735 | 1.00 | 21.48 | A | C |
| ATOM | 795 | CD1 | ILE | 201 | −2.661 | −30.520 | 113.133 | 1.00 | 21.35 | A | C |
| ATOM | 796 | C | ILE | 201 | −4.540 | −35.209 | 112.806 | 1.00 | 21.13 | A | C |
| ATOM | 797 | O | ILE | 201 | −5.659 | −35.413 | 113.278 | 1.00 | 21.64 | A | O |
| ATOM | 798 | N | CYS | 202 | −4.136 | −35.737 | 111.657 | 1.00 | 20.20 | A | N |
| ATOM | 799 | CA | CYS | 202 | −5.007 | −36.617 | 110.893 | 1.00 | 20.37 | A | C |
| ATOM | 800 | CB | CYS | 202 | −4.332 | −37.024 | 109.585 | 1.00 | 20.42 | A | C |
| ATOM | 801 | SG | CYS | 202 | −4.259 | −35.662 | 108.393 | 1.00 | 21.08 | A | S |
| ATOM | 802 | C | CYS | 202 | −5.413 | −37.848 | 111.694 | 1.00 | 18.85 | A | C |
| ATOM | 803 | O | CYS | 202 | −6.582 | −38.234 | 111.687 | 1.00 | 18.40 | A | O |
| ATOM | 804 | N | HIS | 203 | −4.461 | −38.465 | 112.384 | 1.00 | 18.00 | A | N |
| ATOM | 805 | CA | HIS | 203 | −4.781 | −39.635 | 113.190 | 1.00 | 18.29 | A | C |
| ATOM | 806 | CB | HIS | 203 | −3.505 | −40.312 | 113.686 | 1.00 | 17.63 | A | C |
| ATOM | 807 | CG | HIS | 203 | −2.837 | −41.157 | 112.646 | 1.00 | 18.10 | A | C |
| ATOM | 808 | CD2 | HIS | 203 | −1.678 | −40.986 | 111.967 | 1.00 | 17.36 | A | C |
| ATOM | 809 | ND1 | HIS | 203 | −3.398 | −42.318 | 112.161 | 1.00 | 17.64 | A | N |
| ATOM | 810 | CE1 | HIS | 203 | −2.616 | −42.825 | 111.225 | 1.00 | 17.59 | A | C |
| ATOM | 811 | NE2 | HIS | 203 | −1.567 | −42.035 | 111.087 | 1.00 | 17.70 | A | N |
| ATOM | 812 | C | HIS | 203 | −5.690 | −39.255 | 114.361 | 1.00 | 18.57 | A | C |
| ATOM | 813 | O | HIS | 203 | −6.586 | −40.012 | 114.724 | 1.00 | 17.74 | A | O |
| ATOM | 814 | N | ILE | 204 | −5.470 | −38.080 | 114.945 | 1.00 | 17.95 | A | N |
| ATOM | 815 | CA | ILE | 204 | −6.326 | −37.634 | 116.031 | 1.00 | 18.97 | A | C |
| ATOM | 816 | CB | ILE | 204 | −5.867 | −36.271 | 116.595 | 1.00 | 18.95 | A | C |
| ATOM | 817 | CG2 | ILE | 204 | −6.949 | −35.696 | 117.530 | 1.00 | 17.63 | A | C |
| ATOM | 818 | CG1 | ILE | 204 | −4.529 | −36.436 | 117.322 | 1.00 | 17.08 | A | C |
| ATOM | 819 | CD1 | ILE | 204 | −3.990 | −35.142 | 117.913 | 1.00 | 16.88 | A | C |
| ATOM | 820 | C | ILE | 204 | −7.754 | −37.491 | 115.490 | 1.00 | 19.25 | A | C |
| ATOM | 821 | O | ILE | 204 | −8.708 | −37.949 | 116.111 | 1.00 | 19.34 | A | O |
| ATOM | 822 | N | VAL | 205 | −7.890 | −36.859 | 114.327 | 1.00 | 19.23 | A | N |
| ATOM | 823 | CA | VAL | 205 | −9.195 | −36.660 | 113.708 | 1.00 | 19.58 | A | C |
| ATOM | 824 | CB | VAL | 205 | −9.070 | −35.782 | 112.437 | 1.00 | 19.63 | A | C |
| ATOM | 825 | CG1 | VAL | 205 | −10.396 | −35.756 | 111.680 | 1.00 | 20.05 | A | C |
| ATOM | 826 | CG2 | VAL | 205 | −8.666 | −34.371 | 112.823 | 1.00 | 19.24 | A | C |
| ATOM | 827 | C | VAL | 205 | −9.881 | −37.979 | 113.330 | 1.00 | 19.64 | A | C |
| ATOM | 828 | O | VAL | 205 | −11.078 | −38.145 | 113.545 | 1.00 | 19.82 | A | O |
| ATOM | 829 | N | LEU | 206 | −9.112 | −38.911 | 112.773 | 1.00 | 19.63 | A | N |
| ATOM | 830 | CA | LEU | 206 | −9.639 | −40.204 | 112.342 | 1.00 | 19.49 | A | C |
| ATOM | 831 | CB | LEU | 206 | −8.650 | −40.866 | 111.379 | 1.00 | 19.58 | A | C |
| ATOM | 832 | CG | LEU | 206 | −8.980 | −40.934 | 109.879 | 1.00 | 21.14 | A | C |
| ATOM | 833 | CD1 | LEU | 206 | −9.924 | −39.817 | 109.455 | 1.00 | 20.56 | A | C |
| ATOM | 834 | CD2 | LEU | 206 | −7.674 | −40.869 | 109.094 | 1.00 | 20.01 | A | C |
| ATOM | 835 | C | LEU | 206 | −9.977 | −41.175 | 113.469 | 1.00 | 18.64 | A | C |
| ATOM | 836 | O | LEU | 206 | −10.662 | −42.178 | 113.242 | 1.00 | 18.00 | A | O |
| ATOM | 837 | N | ASN | 207 | −9.515 | −40.877 | 114.678 | 1.00 | 17.72 | A | N |
| ATOM | 838 | CA | ASN | 207 | −9.763 | −41.752 | 115.817 | 1.00 | 17.71 | A | C |
| ATOM | 839 | CB | ASN | 207 | −9.148 | −41.162 | 117.089 | 1.00 | 16.46 | A | C |
| ATOM | 840 | CG | ASN | 207 | −9.297 | −42.086 | 118.291 | 1.00 | 17.11 | A | C |
| ATOM | 841 | OD1 | ASN | 207 | −10.037 | −41.792 | 119.233 | 1.00 | 16.94 | A | O |
| ATOM | 842 | ND2 | ASN | 207 | −8.600 | −43.216 | 118.255 | 1.00 | 14.15 | A | N |
| ATOM | 843 | C | ASN | 207 | −11.247 | −42.043 | 116.052 | 1.00 | 18.08 | A | C |
| ATOM | 844 | O | ASN | 207 | −11.601 | −43.148 | 116.479 | 1.00 | 17.24 | A | O |
| ATOM | 845 | N | THR | 208 | −12.117 | −41.069 | 115.782 | 1.00 | 17.73 | A | N |
| ATOM | 846 | CA | THR | 208 | −13.542 | −41.306 | 115.978 | 1.00 | 19.30 | A | C |
| ATOM | 847 | CB | THR | 208 | −14.377 | −40.000 | 115.973 | 1.00 | 19.32 | A | C |
| ATOM | 848 | OG1 | THR | 208 | −13.996 | −39.163 | 114.875 | 1.00 | 20.78 | A | O |
| ATOM | 849 | CG2 | THR | 208 | −14.184 | −39.262 | 117.284 | 1.00 | 20.86 | A | C |
| ATOM | 850 | C | THR | 208 | −14.145 | −42.306 | 114.987 | 1.00 | 19.41 | A | C |
| ATOM | 851 | O | THR | 208 | −15.288 | −42.716 | 115.150 | 1.00 | 19.80 | A | O |
| ATOM | 852 | N | THR | 209 | −13.394 | −42.706 | 113.964 | 1.00 | 19.05 | A | N |
| ATOM | 853 | CA | THR | 209 | −13.909 | −43.722 | 113.045 | 1.00 | 19.58 | A | C |
| ATOM | 854 | CB | THR | 209 | −13.389 | −43.556 | 111.608 | 1.00 | 19.77 | A | C |
| ATOM | 855 | OG1 | THR | 209 | −11.978 | −43.799 | 111.584 | 1.00 | 19.93 | A | O |
| ATOM | 856 | CG2 | THR | 209 | −13.688 | −42.146 | 111.073 | 1.00 | 20.26 | A | C |
| ATOM | 857 | C | THR | 209 | −13.456 | −45.103 | 113.537 | 1.00 | 19.57 | A | C |
| ATOM | 858 | O | THR | 209 | −13.954 | −46.128 | 113.079 | 1.00 | 19.72 | A | O |
| ATOM | 859 | N | PHE | 210 | −12.520 | −45.127 | 114.483 | 1.00 | 19.02 | A | N |
| ATOM | 860 | CA | PHE | 210 | −12.000 | −46.386 | 115.006 | 1.00 | 20.29 | A | C |
| ATOM | 861 | CB | PHE | 210 | −10.765 | −46.127 | 115.873 | 1.00 | 19.30 | A | C |
| ATOM | 862 | CG | PHE | 210 | −9.938 | −47.354 | 116.129 | 1.00 | 19.82 | A | C |
| ATOM | 863 | CD1 | PHE | 210 | −9.215 | −47.948 | 115.096 | 1.00 | 19.46 | A | C |
| ATOM | 864 | CD2 | PHE | 210 | −9.891 | −47.926 | 117.399 | 1.00 | 18.74 | A | C |
| ATOM | 865 | CE1 | PHE | 210 | −8.454 | −49.096 | 115.325 | 1.00 | 19.60 | A | C |
| ATOM | 866 | CE2 | PHE | 210 | −9.135 | −49.072 | 117.641 | 1.00 | 19.21 | A | C |
| ATOM | 867 | CZ | PHE | 210 | −8.415 | −49.661 | 116.605 | 1.00 | 19.22 | A | C |
| ATOM | 868 | C | PHE | 210 | −13.035 | −47.176 | 115.810 | 1.00 | 21.23 | A | C |
| ATOM | 869 | O | PHE | 210 | −13.639 | −46.667 | 116.756 | 1.00 | 20.44 | A | O |
| ATOM | 870 | N | CYS | 211 | −13.229 | −48.430 | 115.421 | 1.00 | 22.24 | A | N |
| ATOM | 871 | CA | CYS | 211 | −14.175 | −49.304 | 116.087 | 1.00 | 24.03 | A | C |
| ATOM | 872 | CB | CYS | 211 | −14.950 | −50.117 | 115.049 | 1.00 | 24.67 | A | C |
| ATOM | 873 | SG | CYS | 211 | −16.182 | −51.252 | 115.747 | 1.00 | 25.21 | A | S |
| ATOM | 874 | C | CYS | 211 | −13.385 | −50.228 | 117.006 | 1.00 | 24.78 | A | C |
| ATOM | 875 | O | CYS | 211 | −12.508 | −50.962 | 116.562 | 1.00 | 23.56 | A | O |
| ATOM | 876 | N | LEU | 212 | −13.690 | −50.179 | 118.294 | 1.00 | 25.72 | A | N |
| ATOM | 877 | CA | LEU | 212 | −12.989 | −51.006 | 119.262 | 1.00 | 27.59 | A | C |
| ATOM | 878 | CB | LEU | 212 | −13.380 | −50.578 | 120.671 | 1.00 | 27.74 | A | C |
| ATOM | 879 | CG | LEU | 212 | −12.881 | −49.185 | 121.058 | 1.00 | 28.59 | A | C |
| ATOM | 880 | CD1 | LEU | 212 | −13.486 | −48.759 | 122.396 | 1.00 | 28.93 | A | C |
| ATOM | 881 | CD2 | LEU | 212 | −11.365 | −49.210 | 121.125 | 1.00 | 27.26 | A | C |
| ATOM | 882 | C | LEU | 212 | −13.272 | −52.489 | 119.075 | 1.00 | 29.09 | A | C |
| ATOM | 883 | O | LEU | 212 | −12.374 | −53.322 | 119.189 | 1.00 | 28.72 | A | O |
| ATOM | 884 | N | GLN | 213 | −14.524 | −52.805 | 118.765 | 1.00 | 30.49 | A | N |
| ATOM | 885 | CA | GLN | 213 | −14.956 | −54.181 | 118.583 | 1.00 | 32.47 | A | C |
| ATOM | 886 | CB | GLN | 213 | −16.457 | −54.204 | 118.283 | 1.00 | 35.49 | A | C |
| ATOM | 887 | CG | GLN | 213 | −17.157 | −55.523 | 118.581 | 1.00 | 40.38 | A | C |
| ATOM | 888 | CD | GLN | 213 | −17.315 | −55.780 | 120.073 | 1.00 | 42.74 | A | C |
| ATOM | 889 | OE1 | GLN | 213 | −17.855 | −54.945 | 120.806 | 1.00 | 44.72 | A | O |
| ATOM | 890 | NE2 | GLN | 213 | −16.849 | −56.939 | 120.528 | 1.00 | 44.13 | A | N |
| ATOM | 891 | C | GLN | 213 | −14.200 | −54.911 | 117.474 | 1.00 | 31.71 | A | C |
| ATOM | 892 | O | GLN | 213 | −13.745 | −56.034 | 117.662 | 1.00 | 31.33 | A | O |
| ATOM | 893 | N | THR | 214 | −14.068 | −54.272 | 116.319 | 1.00 | 30.92 | A | N |
| ATOM | 894 | CA | THR | 214 | −13.388 | −54.888 | 115.183 | 1.00 | 30.21 | A | C |
| ATOM | 895 | CB | THR | 214 | −14.189 | −54.681 | 113.898 | 1.00 | 30.65 | A | C |
| ATOM | 896 | OG1 | THR | 214 | −14.410 | −53.278 | 113.707 | 1.00 | 30.08 | A | O |
| ATOM | 897 | CG2 | THR | 214 | −15.528 | −55.404 | 113.983 | 1.00 | 30.64 | A | C |
| ATOM | 898 | C | THR | 214 | −11.971 | −54.390 | 114.922 | 1.00 | 29.58 | A | C |
| ATOM | 899 | O | THR | 214 | −11.272 | −54.943 | 114.079 | 1.00 | 29.04 | A | O |
| ATOM | 900 | N | GLN | 215 | −11.555 | −53.341 | 115.625 | 1.00 | 29.13 | A | N |
| ATOM | 901 | CA | GLN | 215 | −10.214 | −52.788 | 115.454 | 1.00 | 29.85 | A | C |
| ATOM | 902 | CB | GLN | 215 | −9.179 | −53.869 | 115.775 | 1.00 | 31.30 | A | C |
| ATOM | 903 | CG | GLN | 215 | −7.916 | −53.384 | 116.476 | 1.00 | 35.10 | A | C |
| ATOM | 904 | CD | GLN | 215 | −8.150 | −52.959 | 117.919 | 1.00 | 36.36 | A | C |
| ATOM | 905 | OE1 | GLN | 215 | −9.137 | −53.351 | 118.547 | 1.00 | 36.52 | A | O |
| ATOM | 906 | NE2 | GLN | 215 | −7.228 | −52.163 | 118.456 | 1.00 | 37.92 | A | N |
| ATOM | 907 | C | GLN | 215 | −10.045 | −52.300 | 114.007 | 1.00 | 29.28 | A | C |
| ATOM | 908 | O | GLN | 215 | −8.980 | −52.457 | 113.397 | 1.00 | 29.33 | A | O |
| ATOM | 909 | N | ASN | 216 | −11.110 | −51.715 | 113.472 | 1.00 | 27.92 | A | N |
| ATOM | 910 | CA | ASN | 216 | −11.138 | −51.208 | 112.104 | 1.00 | 27.75 | A | C |
| ATOM | 911 | CB | ASN | 216 | −12.198 | −51.953 | 111.276 | 1.00 | 29.04 | A | C |
| ATOM | 912 | CG | ASN | 216 | −11.820 | −53.388 | 110.966 | 1.00 | 31.13 | A | C |
| ATOM | 913 | OD1 | ASN | 216 | −12.672 | −54.183 | 110.566 | 1.00 | 31.15 | A | O |
| ATOM | 914 | ND2 | ASN | 216 | −10.544 | −53.728 | 111.130 | 1.00 | 32.04 | A | N |
| ATOM | 915 | C | ASN | 216 | −11.521 | −49.739 | 112.109 | 1.00 | 26.04 | A | C |
| ATOM | 916 | O | ASN | 216 | −12.077 | −49.236 | 113.079 | 1.00 | 25.94 | A | O |
| ATOM | 917 | N | PHE | 217 | −11.226 | −49.057 | 111.012 | 1.00 | 24.54 | A | N |
| ATOM | 918 | CA | PHE | 217 | −11.607 | −47.660 | 110.868 | 1.00 | 23.68 | A | C |
| ATOM | 919 | CB | PHE | 217 | −10.474 | −46.846 | 110.247 | 1.00 | 21.91 | A | C |
| ATOM | 920 | CG | PHE | 217 | −9.271 | −46.723 | 111.132 | 1.00 | 21.44 | A | C |
| ATOM | 921 | CD1 | PHE | 217 | −8.295 | −47.709 | 111.143 | 1.00 | 20.23 | A | C |
| ATOM | 922 | CD2 | PHE | 217 | −9.127 | −45.625 | 111.977 | 1.00 | 21.42 | A | C |
| ATOM | 923 | CE1 | PHE | 217 | −7.191 | −47.603 | 111.982 | 1.00 | 21.65 | A | C |
| ATOM | 924 | CE2 | PHE | 217 | −8.024 | −45.509 | 112.825 | 1.00 | 21.20 | A | C |
| ATOM | 925 | CZ | PHE | 217 | −7.057 | −46.497 | 112.826 | 1.00 | 20.69 | A | C |
| ATOM | 926 | C | PHE | 217 | −12.821 | −47.677 | 109.944 | 1.00 | 23.24 | A | C |
| ATOM | 927 | O | PHE | 217 | −12.714 | −48.053 | 108.778 | 1.00 | 23.15 | A | O |
| ATOM | 928 | N | LEU | 218 | −13.976 | −47.292 | 110.475 | 1.00 | 22.85 | A | N |
| ATOM | 929 | CA | LEU | 218 | −15.217 | −47.292 | 109.697 | 1.00 | 22.28 | A | C |
| ATOM | 930 | CB | LEU | 218 | −16.388 | −47.702 | 110.591 | 1.00 | 22.69 | A | C |
| ATOM | 931 | CG | LEU | 218 | −16.185 | −49.017 | 111.344 | 1.00 | 24.13 | A | C |
| ATOM | 932 | CD1 | LEU | 218 | −17.413 | −49.316 | 112.191 | 1.00 | 24.57 | A | C |
| ATOM | 933 | CD2 | LEU | 218 | −15.923 | −50.148 | 110.346 | 1.00 | 24.80 | A | C |
| ATOM | 934 | C | LEU | 218 | −15.478 | −45.919 | 109.110 | 1.00 | 21.34 | A | C |
| ATOM | 935 | O | LEU | 218 | −15.830 | −44.984 | 109.830 | 1.00 | 20.56 | A | O |
| ATOM | 936 | N | CYS | 219 | −15.305 | −45.805 | 107.798 | 1.00 | 21.05 | A | N |
| ATOM | 937 | CA | CYS | 219 | −15.502 | −44.541 | 107.101 | 1.00 | 20.80 | A | C |
| ATOM | 938 | CB | CYS | 219 | −14.203 | −44.136 | 106.399 | 1.00 | 20.03 | A | C |
| ATOM | 939 | SG | CYS | 219 | −12.762 | −44.055 | 107.502 | 1.00 | 21.17 | A | S |
| ATOM | 940 | C | CYS | 219 | −16.640 | −44.667 | 106.087 | 1.00 | 21.14 | A | C |
| ATOM | 941 | O | CYS | 219 | −16.414 | −44.904 | 104.889 | 1.00 | 20.85 | A | O |
| ATOM | 942 | N | GLY | 220 | −17.865 | −44.492 | 106.574 | 1.00 | 21.18 | A | N |
| ATOM | 943 | CA | GLY | 220 | −19.024 | −44.612 | 105.710 | 1.00 | 21.35 | A | C |
| ATOM | 944 | C | GLY | 220 | −19.104 | −46.047 | 105.222 | 1.00 | 21.61 | A | C |
| ATOM | 945 | O | GLY | 220 | −19.079 | −46.971 | 106.025 | 1.00 | 21.96 | A | O |
| ATOM | 946 | N | PRO | 221 | −19.197 | −46.270 | 103.908 | 1.00 | 21.46 | A | N |
| ATOM | 947 | CD | PRO | 221 | −19.369 | −45.295 | 102.816 | 1.00 | 21.21 | A | C |
| ATOM | 948 | CA | PRO | 221 | −19.273 | −47.640 | 103.402 | 1.00 | 22.15 | A | C |
| ATOM | 949 | CB | PRO | 221 | −19.909 | −47.451 | 102.027 | 1.00 | 21.42 | A | C |
| ATOM | 950 | CG | PRO | 221 | −19.277 | −46.164 | 101.576 | 1.00 | 20.84 | A | C |
| ATOM | 951 | C | PRO | 221 | −17.893 | −48.313 | 103.320 | 1.00 | 22.19 | A | C |
| ATOM | 952 | O | PRO | 221 | −17.794 | −49.481 | 102.938 | 1.00 | 22.79 | A | O |
| ATOM | 953 | N | LEU | 222 | −16.841 | −47.577 | 103.678 | 1.00 | 21.79 | A | N |
| ATOM | 954 | CA | LEU | 222 | −15.473 | −48.097 | 103.633 | 1.00 | 21.31 | A | C |
| ATOM | 955 | CB | LEU | 222 | −14.511 | −47.032 | 103.093 | 1.00 | 19.09 | A | C |
| ATOM | 956 | CG | LEU | 222 | −14.746 | −46.584 | 101.644 | 1.00 | 18.95 | A | C |
| ATOM | 957 | CD1 | LEU | 222 | −13.870 | −45.392 | 101.319 | 1.00 | 18.64 | A | C |
| ATOM | 958 | CD2 | LEU | 222 | −14.460 | −47.738 | 100.691 | 1.00 | 16.82 | A | C |
| ATOM | 959 | C | LEU | 222 | −14.985 | −48.569 | 104.999 | 1.00 | 21.86 | A | C |
| ATOM | 960 | O | LEU | 222 | −15.313 | −47.974 | 106.030 | 1.00 | 21.48 | A | O |
| ATOM | 961 | N | ARG | 223 | −14.184 | −49.634 | 104.979 | 1.00 | 21.57 | A | N |
| ATOM | 962 | CA | ARG | 223 | −13.618 | −50.249 | 106.175 | 1.00 | 22.37 | A | C |
| ATOM | 963 | CB | ARG | 223 | −14.377 | −51.558 | 106.465 | 1.00 | 24.98 | A | C |
| ATOM | 964 | CG | ARG | 223 | −13.739 | −52.476 | 107.490 | 1.00 | 28.92 | A | C |
| ATOM | 965 | CD | ARG | 223 | −13.141 | −53.746 | 106.858 | 1.00 | 32.82 | A | C |
| ATOM | 966 | NE | ARG | 223 | −14.154 | −54.668 | 106.324 | 1.00 | 36.01 | A | N |
| ATOM | 967 | CZ | ARG | 223 | −14.568 | −54.692 | 105.057 | 1.00 | 36.66 | A | C |
| ATOM | 968 | NH1 | ARG | 223 | −14.058 | −53.847 | 104.167 | 1.00 | 36.15 | A | N |
| ATOM | 969 | NH2 | ARG | 223 | −15.495 | −55.565 | 104.677 | 1.00 | 36.63 | A | N |
| ATOM | 970 | C | ARG | 223 | −12.113 | −50.515 | 105.967 | 1.00 | 21.18 | A | C |
| ATOM | 971 | O | ARG | 223 | −11.718 | −51.260 | 105.070 | 1.00 | 20.16 | A | O |
| ATOM | 972 | N | TYR | 224 | −11.277 | −49.884 | 106.782 | 1.00 | 18.92 | A | N |
| ATOM | 973 | CA | TYR | 224 | −9.829 | −50.070 | 106.678 | 1.00 | 19.29 | A | C |
| ATOM | 974 | CB | TYR | 224 | −9.088 | −48.723 | 106.716 | 1.00 | 17.44 | A | C |
| ATOM | 975 | CG | TYR | 224 | −9.470 | −47.770 | 105.604 | 1.00 | 17.07 | A | C |
| ATOM | 976 | CD1 | TYR | 224 | −10.560 | −46.908 | 105.741 | 1.00 | 16.45 | A | C |
| ATOM | 977 | CE1 | TYR | 224 | −10.936 | −46.053 | 104.711 | 1.00 | 16.85 | A | C |
| ATOM | 978 | CD2 | TYR | 224 | −8.761 | −47.751 | 104.402 | 1.00 | 16.10 | A | C |
| ATOM | 979 | CE2 | TYR | 224 | −9.131 | −46.897 | 103.357 | 1.00 | 16.65 | A | C |
| ATOM | 980 | CZ | TYR | 224 | −10.225 | −46.052 | 103.521 | 1.00 | 16.90 | A | C |
| ATOM | 981 | OH | TYR | 224 | −10.636 | −45.234 | 102.490 | 1.00 | 16.57 | A | O |
| ATOM | 982 | C | TYR | 224 | −9.354 | −50.949 | 107.836 | 1.00 | 18.68 | A | C |
| ATOM | 983 | O | TYR | 224 | −9.712 | −50.713 | 108.986 | 1.00 | 18.12 | A | O |
| ATOM | 984 | N | THR | 225 | −8.543 | −51.951 | 107.516 | 1.00 | 17.86 | A | N |
| ATOM | 985 | CA | THR | 225 | −8.029 | −52.894 | 108.505 | 1.00 | 18.12 | A | C |
| ATOM | 986 | CB | THR | 225 | −8.322 | −54.355 | 108.091 | 1.00 | 16.87 | A | C |
| ATOM | 987 | OG1 | THR | 225 | −7.612 | −54.637 | 106.883 | 1.00 | 16.75 | A | O |
| ATOM | 988 | CG2 | THR | 225 | −9.803 | −54.578 | 107.848 | 1.00 | 15.99 | A | C |
| ATOM | 989 | C | THR | 225 | −6.514 | −52.781 | 108.624 | 1.00 | 17.74 | A | C |
| ATOM | 990 | O | THR | 225 | −5.855 | −52.164 | 107.785 | 1.00 | 17.42 | A | O |
| ATOM | 991 | N | ILE | 226 | −5.964 | −53.404 | 109.660 | 1.00 | 16.95 | A | N |
| ATOM | 992 | CA | ILE | 226 | −4.526 | −53.390 | 109.861 | 1.00 | 17.61 | A | C |
| ATOM | 993 | CB | ILE | 226 | −4.161 | −53.985 | 111.246 | 1.00 | 17.39 | A | C |
| ATOM | 994 | CG2 | ILE | 226 | −4.492 | −55.476 | 111.290 | 1.00 | 15.65 | A | C |
| ATOM | 995 | CG1 | ILE | 226 | −2.685 | −53.713 | 111.555 | 1.00 | 17.84 | A | C |
| ATOM | 996 | CD1 | ILE | 226 | −2.309 | −54.002 | 112.989 | 1.00 | 18.23 | A | C |
| ATOM | 997 | C | ILE | 226 | −3.823 | −54.153 | 108.714 | 1.00 | 17.93 | A | C |
| ATOM | 998 | O | ILE | 226 | −2.662 | −53.874 | 108.403 | 1.00 | 16.65 | A | O |
| ATOM | 999 | N | GLU | 227 | −4.530 | −55.089 | 108.072 | 1.00 | 17.96 | A | N |
| ATOM | 1000 | CA | GLU | 227 | −3.962 | −55.840 | 106.949 | 1.00 | 19.11 | A | C |
| ATOM | 1001 | CB | GLU | 227 | −4.891 | −56.981 | 106.503 | 1.00 | 20.72 | A | C |
| ATOM | 1002 | CG | GLU | 227 | −4.912 | −58.231 | 107.401 | 1.00 | 20.80 | A | C |
| ATOM | 1003 | CD | GLU | 227 | −5.526 | −57.975 | 108.761 | 1.00 | 21.93 | A | C |
| ATOM | 1004 | OE1 | GLU | 227 | −6.513 | −57.220 | 108.833 | 1.00 | 23.31 | A | O |
| ATOM | 1005 | OE2 | GLU | 227 | −5.040 | −58.536 | 109.760 | 1.00 | 21.48 | A | O |
| ATOM | 1006 | C | GLU | 227 | −3.718 | −54.914 | 105.757 | 1.00 | 19.73 | A | C |
| ATOM | 1007 | O | GLU | 227 | −2.805 | −55.147 | 104.954 | 1.00 | 18.85 | A | O |
| ATOM | 1008 | N | ASP | 228 | −4.546 | −53.877 | 105.623 | 1.00 | 18.55 | A | N |
| ATOM | 1009 | CA | ASP | 228 | −4.371 | −52.928 | 104.533 | 1.00 | 19.17 | A | C |
| ATOM | 1010 | CB | ASP | 228 | −5.504 | −51.886 | 104.522 | 1.00 | 19.08 | A | C |
| ATOM | 1011 | CG | ASP | 228 | −6.846 | −52.496 | 104.159 | 1.00 | 19.07 | A | C |
| ATOM | 1012 | OD1 | ASP | 228 | −6.873 | −53.316 | 103.219 | 1.00 | 20.93 | A | O |
| ATOM | 1013 | OD2 | ASP | 228 | −7.869 | −52.164 | 104.795 | 1.00 | 18.63 | A | O |
| ATOM | 1014 | C | ASP | 228 | −3.012 | −52.251 | 104.691 | 1.00 | 18.73 | A | C |
| ATOM | 1015 | O | ASP | 228 | −2.279 | −52.077 | 103.715 | 1.00 | 19.17 | A | O |
| ATOM | 1016 | N | GLY | 229 | −2.672 | −51.879 | 105.922 | 1.00 | 18.10 | A | N |
| ATOM | 1017 | CA | GLY | 229 | −1.386 | −51.253 | 106.164 | 1.00 | 17.50 | A | C |
| ATOM | 1018 | C | GLY | 229 | −0.245 | −52.236 | 105.926 | 1.00 | 17.22 | A | C |
| ATOM | 1019 | O | GLY | 229 | 0.771 | −51.898 | 105.321 | 1.00 | 15.48 | A | O |
| ATOM | 1020 | N | ALA | 230 | −0.413 | −53.461 | 106.410 | 1.00 | 17.33 | A | N |
| ATOM | 1021 | CA | ALA | 230 | 0.608 | −54.485 | 106.247 | 1.00 | 18.15 | A | C |
| ATOM | 1022 | CB | ALA | 230 | 0.195 | −55.745 | 106.987 | 1.00 | 16.66 | A | C |
| ATOM | 1023 | C | ALA | 230 | 0.855 | −54.795 | 104.770 | 1.00 | 19.00 | A | C |
| ATOM | 1024 | O | ALA | 230 | 2.001 | −54.934 | 104.344 | 1.00 | 19.76 | A | O |
| ATOM | 1025 | N | ARG | 231 | −0.219 | −54.887 | 103.990 | 1.00 | 19.49 | A | N |
| ATOM | 1026 | CA | ARG | 231 | −0.109 | −55.193 | 102.566 | 1.00 | 19.81 | A | C |
| ATOM | 1027 | CB | ARG | 231 | −1.491 | −55.499 | 101.973 | 1.00 | 20.78 | A | C |
| ATOM | 1028 | CG | ARG | 231 | −2.159 | −56.765 | 102.524 | 1.00 | 22.44 | A | C |
| ATOM | 1029 | CD | ARG | 231 | −1.366 | −58.040 | 102.205 | 1.00 | 24.31 | A | C |
| ATOM | 1030 | NE | ARG | 231 | −1.373 | −58.385 | 100.783 | 1.00 | 26.32 | A | N |
| ATOM | 1031 | CZ | ARG | 231 | −2.383 | −58.974 | 100.143 | 1.00 | 28.16 | A | C |
| ATOM | 1032 | NH1 | ARG | 231 | −3.498 | −59.305 | 100.780 | 1.00 | 27.73 | A | N |
| ATOM | 1033 | NH2 | ARG | 231 | −2.276 | −59.233 | 98.846 | 1.00 | 30.42 | A | N |
| ATOM | 1034 | C | ARG | 231 | 0.590 | −54.127 | 101.721 | 1.00 | 19.74 | A | C |
| ATOM | 1035 | O | ARG | 231 | 1.107 | −54.451 | 100.650 | 1.00 | 20.20 | A | O |
| ATOM | 1036 | N | VAL | 232 | 0.608 | −52.867 | 102.170 | 1.00 | 18.61 | A | N |
| ATOM | 1037 | CA | VAL | 232 | 1.296 | −51.826 | 101.404 | 1.00 | 17.26 | A | C |
| ATOM | 1038 | CB | VAL | 232 | 0.569 | −50.434 | 101.462 | 1.00 | 17.52 | A | C |
| ATOM | 1039 | CG1 | VAL | 232 | −0.855 | −50.569 | 100.919 | 1.00 | 17.25 | A | C |
| ATOM | 1040 | CG2 | VAL | 232 | 0.563 | −49.875 | 102.879 | 1.00 | 16.49 | A | C |
| ATOM | 1041 | C | VAL | 232 | 2.748 | −51.670 | 101.852 | 1.00 | 17.95 | A | C |
| ATOM | 1042 | O | VAL | 232 | 3.469 | −50.800 | 101.358 | 1.00 | 17.18 | A | O |
| ATOM | 1043 | N | GLY | 233 | 3.189 | −52.510 | 102.788 | 1.00 | 18.73 | A | N |
| ATOM | 1044 | CA | GLY | 233 | 4.582 | −52.442 | 103.205 | 1.00 | 19.65 | A | C |
| ATOM | 1045 | C | GLY | 233 | 4.955 | −51.997 | 104.608 | 1.00 | 20.28 | A | C |
| ATOM | 1046 | O | GLY | 233 | 6.129 | −52.078 | 104.969 | 1.00 | 20.44 | A | O |
| ATOM | 1047 | N | PHE | 234 | 3.999 | −51.514 | 105.397 | 1.00 | 19.59 | A | N |
| ATOM | 1048 | CA | PHE | 234 | 4.313 | −51.107 | 106.764 | 1.00 | 20.47 | A | C |
| ATOM | 1049 | CB | PHE | 234 | 3.134 | −50.366 | 107.401 | 1.00 | 20.71 | A | C |
| ATOM | 1050 | CG | PHE | 234 | 2.949 | −48.963 | 106.901 | 1.00 | 21.67 | A | C |
| ATOM | 1051 | CD1 | PHE | 234 | 1.790 | −48.602 | 106.229 | 1.00 | 21.54 | A | C |
| ATOM | 1052 | CD2 | PHE | 234 | 3.922 | −47.996 | 107.133 | 1.00 | 22.13 | A | C |
| ATOM | 1053 | CE1 | PHE | 234 | 1.596 | −47.291 | 105.794 | 1.00 | 23.35 | A | C |
| ATOM | 1054 | CE2 | PHE | 234 | 3.741 | −46.681 | 106.703 | 1.00 | 23.24 | A | C |
| ATOM | 1055 | CZ | PHE | 234 | 2.572 | −46.328 | 106.032 | 1.00 | 22.54 | A | C |
| ATOM | 1056 | C | PHE | 234 | 4.648 | −52.322 | 107.633 | 1.00 | 20.13 | A | C |
| ATOM | 1057 | O | PHE | 234 | 4.039 | −53.388 | 107.503 | 1.00 | 19.24 | A | O |
| ATOM | 1058 | N | GLN | 235 | 5.606 | −52.153 | 108.534 | 1.00 | 20.97 | A | N |
| ATOM | 1059 | CA | GLN | 235 | 5.997 | −53.231 | 109.427 | 1.00 | 22.42 | A | C |
| ATOM | 1060 | CB | GLN | 235 | 7.348 | −52.917 | 110.064 | 1.00 | 24.45 | A | C |
| ATOM | 1061 | CG | GLN | 235 | 8.493 | −52.968 | 109.069 | 1.00 | 27.92 | A | C |
| ATOM | 1062 | CD | GLN | 235 | 9.841 | −52.727 | 109.709 | 1.00 | 30.93 | A | C |
| ATOM | 1063 | OE1 | GLN | 235 | 10.843 | −53.322 | 109.304 | 1.00 | 33.56 | A | O |
| ATOM | 1064 | NE2 | GLN | 235 | 9.883 | −51.847 | 110.703 | 1.00 | 30.93 | A | N |
| ATOM | 1065 | C | GLN | 235 | 4.932 | −53.444 | 110.494 | 1.00 | 21.69 | A | C |
| ATOM | 1066 | O | GLN | 235 | 4.303 | −52.498 | 110.966 | 1.00 | 20.35 | A | O |
| ATOM | 1067 | N | VAL | 236 | 4.729 | −54.702 | 110.863 | 1.00 | 21.75 | A | N |
| ATOM | 1068 | CA | VAL | 236 | 3.725 | −55.064 | 111.850 | 1.00 | 20.74 | A | C |
| ATOM | 1069 | CB | VAL | 236 | 3.679 | −56.585 | 112.035 | 1.00 | 20.71 | A | C |
| ATOM | 1070 | CG1 | VAL | 236 | 2.728 | −56.953 | 113.178 | 1.00 | 19.30 | A | C |
| ATOM | 1071 | CG2 | VAL | 236 | 3.233 | −57.231 | 110.730 | 1.00 | 20.83 | A | C |
| ATOM | 1072 | C | VAL | 236 | 3.906 | −54.393 | 113.197 | 1.00 | 21.19 | A | C |
| ATOM | 1073 | O | VAL | 236 | 2.933 | −53.928 | 113.785 | 1.00 | 20.57 | A | O |
| ATOM | 1074 | N | GLU | 237 | 5.138 | −54.346 | 113.695 | 1.00 | 21.62 | A | N |
| ATOM | 1075 | CA | GLU | 237 | 5.400 | −53.702 | 114.981 | 1.00 | 23.17 | A | C |
| ATOM | 1076 | CB | GLU | 237 | 6.890 | −53.788 | 115.323 | 1.00 | 25.38 | A | C |
| ATOM | 1077 | CG | GLU | 237 | 7.309 | −52.979 | 116.535 | 1.00 | 29.56 | A | C |
| ATOM | 1078 | CD | GLU | 237 | 8.719 | −53.315 | 117.002 | 1.00 | 32.34 | A | C |
| ATOM | 1079 | OE1 | GLU | 237 | 9.616 | −53.489 | 116.146 | 1.00 | 34.48 | A | O |
| ATOM | 1080 | OE2 | GLU | 237 | 8.931 | −53.400 | 118.227 | 1.00 | 33.42 | A | O |
| ATOM | 1081 | C | GLU | 237 | 4.940 | −52.241 | 114.945 | 1.00 | 22.30 | A | C |
| ATOM | 1082 | O | GLU | 237 | 4.354 | −51.745 | 115.905 | 1.00 | 21.24 | A | O |
| ATOM | 1083 | N | PHE | 238 | 5.210 | −51.558 | 113.836 | 1.00 | 21.77 | A | N |
| ATOM | 1084 | CA | PHE | 238 | 4.779 | −50.169 | 113.667 | 1.00 | 22.10 | A | C |
| ATOM | 1085 | CB | PHE | 238 | 5.284 | −49.611 | 112.331 | 1.00 | 21.91 | A | C |
| ATOM | 1086 | CG | PHE | 238 | 4.661 | −48.296 | 111.954 | 1.00 | 22.44 | A | C |
| ATOM | 1087 | CD1 | PHE | 238 | 5.015 | −47.123 | 112.622 | 1.00 | 22.09 | A | C |
| ATOM | 1088 | CD2 | PHE | 238 | 3.687 | −48.234 | 110.961 | 1.00 | 22.65 | A | C |
| ATOM | 1089 | CE1 | PHE | 238 | 4.409 | −45.914 | 112.310 | 1.00 | 21.36 | A | C |
| ATOM | 1090 | CE2 | PHE | 238 | 3.073 | −47.026 | 110.636 | 1.00 | 22.78 | A | C |
| ATOM | 1091 | CZ | PHE | 238 | 3.436 | −45.861 | 111.316 | 1.00 | 22.50 | A | C |
| ATOM | 1092 | C | PHE | 238 | 3.245 | −50.142 | 113.673 | 1.00 | 21.89 | A | C |
| ATOM | 1093 | O | PHE | 238 | 2.618 | −49.354 | 114.378 | 1.00 | 20.84 | A | O |
| ATOM | 1094 | N | LEU | 239 | 2.653 | −51.013 | 112.863 | 1.00 | 22.09 | A | N |
| ATOM | 1095 | CA | LEU | 239 | 1.205 | −51.118 | 112.759 | 1.00 | 22.46 | A | C |
| ATOM | 1096 | CB | LEU | 239 | 0.845 | −52.241 | 111.778 | 1.00 | 21.56 | A | C |
| ATOM | 1097 | CG | LEU | 239 | 0.402 | −51.905 | 110.343 | 1.00 | 23.54 | A | C |
| ATOM | 1098 | CD1 | LEU | 239 | 0.860 | −50.519 | 109.926 | 1.00 | 22.90 | A | C |
| ATOM | 1099 | CD2 | LEU | 239 | 0.925 | −52.977 | 109.388 | 1.00 | 21.20 | A | C |
| ATOM | 1100 | C | LEU | 239 | 0.577 | −51.376 | 114.132 | 1.00 | 22.41 | A | C |
| ATOM | 1101 | O | LEU | 239 | −0.441 | −50.776 | 114.470 | 1.00 | 21.62 | A | O |
| ATOM | 1102 | N | GLU | 240 | 1.182 | −52.262 | 114.923 | 1.00 | 22.26 | A | N |
| ATOM | 1103 | CA | GLU | 240 | 0.668 | −52.563 | 116.256 | 1.00 | 22.89 | A | C |
| ATOM | 1104 | CB | GLU | 240 | 1.471 | −53.690 | 116.918 | 1.00 | 25.34 | A | C |
| ATOM | 1105 | CG | GLU | 240 | 1.267 | −55.069 | 116.305 | 1.00 | 29.77 | A | C |
| ATOM | 1106 | CD | GLU | 240 | −0.189 | −55.498 | 116.294 | 1.00 | 33.75 | A | C |
| ATOM | 1107 | OE1 | GLU | 240 | −1.022 | −54.811 | 116.927 | 1.00 | 36.81 | A | O |
| ATOM | 1108 | OE2 | GLU | 240 | −0.507 | −56.527 | 115.658 | 1.00 | 35.46 | A | O |
| ATOM | 1109 | C | GLU | 240 | 0.705 | −51.327 | 117.151 | 1.00 | 21.32 | A | C |
| ATOM | 1110 | O | GLU | 240 | −0.224 | −51.081 | 117.906 | 1.00 | 20.62 | A | O |
| ATOM | 1111 | N | LEU | 241 | 1.780 | −50.554 | 117.070 | 1.00 | 20.96 | A | N |
| ATOM | 1112 | CA | LEU | 241 | 1.888 | −49.349 | 117.883 | 1.00 | 22.45 | A | C |
| ATOM | 1113 | CB | LEU | 241 | 3.239 | −48.664 | 117.648 | 1.00 | 24.05 | A | C |
| ATOM | 1114 | CG | LEU | 241 | 3.466 | −47.321 | 118.363 | 1.00 | 26.05 | A | C |
| ATOM | 1115 | CD1 | LEU | 241 | 3.433 | −47.531 | 119.877 | 1.00 | 27.21 | A | C |
| ATOM | 1116 | CD2 | LEU | 241 | 4.806 | −46.727 | 117.945 | 1.00 | 26.89 | A | C |
| ATOM | 1117 | C | LEU | 241 | 0.757 | −48.389 | 117.511 | 1.00 | 21.92 | A | C |
| ATOM | 1118 | O | LEU | 241 | 0.067 | −47.855 | 118.381 | 1.00 | 21.86 | A | O |
| ATOM | 1119 | N | LEU | 242 | 0.572 | −48.194 | 116.210 | 1.00 | 20.84 | A | N |
| ATOM | 1120 | CA | LEU | 242 | −0.452 | −47.303 | 115.677 | 1.00 | 20.79 | A | C |
| ATOM | 1121 | CB | LEU | 242 | −0.433 | −47.341 | 114.146 | 1.00 | 19.93 | A | C |
| ATOM | 1122 | CG | LEU | 242 | −0.682 | −46.031 | 113.392 | 1.00 | 21.77 | A | C |
| ATOM | 1123 | CD1 | LEU | 242 | −1.129 | −46.358 | 111.984 | 1.00 | 19.10 | A | C |
| ATOM | 1124 | CD2 | LEU | 242 | −1.729 | −45.184 | 114.085 | 1.00 | 21.40 | A | C |
| ATOM | 1125 | C | LEU | 242 | −1.857 | −47.667 | 116.161 | 1.00 | 20.42 | A | C |
| ATOM | 1126 | O | LEU | 242 | −2.585 | −46.821 | 116.678 | 1.00 | 18.91 | A | O |
| ATOM | 1127 | N | PHE | 243 | −2.231 | −48.929 | 115.981 | 1.00 | 20.27 | A | N |
| ATOM | 1128 | CA | PHE | 243 | −3.545 | −49.390 | 116.386 | 1.00 | 20.87 | A | C |
| ATOM | 1129 | CB | PHE | 243 | −3.828 | −50.766 | 115.775 | 1.00 | 21.22 | A | C |
| ATOM | 1130 | CG | PHE | 243 | −4.211 | −50.704 | 114.309 | 1.00 | 21.05 | A | C |
| ATOM | 1131 | CD1 | PHE | 243 | −3.316 | −50.212 | 113.359 | 1.00 | 19.68 | A | C |
| ATOM | 1132 | CD2 | PHE | 243 | −5.478 | −51.092 | 113.893 | 1.00 | 19.34 | A | C |
| ATOM | 1133 | CE1 | PHE | 243 | −3.678 | −50.104 | 112.022 | 1.00 | 20.59 | A | C |
| ATOM | 1134 | CE2 | PHE | 243 | −5.850 | −50.987 | 112.558 | 1.00 | 19.75 | A | C |
| ATOM | 1135 | CZ | PHE | 243 | −4.950 | −50.492 | 111.621 | 1.00 | 20.16 | A | C |
| ATOM | 1136 | C | PHE | 243 | −3.735 | −49.402 | 117.899 | 1.00 | 21.36 | A | C |
| ATOM | 1137 | O | PHE | 243 | −4.855 | −49.217 | 118.385 | 1.00 | 20.70 | A | O |
| ATOM | 1138 | N | HIS | 244 | −2.652 | −49.608 | 118.644 | 1.00 | 21.51 | A | N |
| ATOM | 1139 | CA | HIS | 244 | −2.747 | −49.586 | 120.101 | 1.00 | 22.59 | A | C |
| ATOM | 1140 | CB | HIS | 244 | −1.448 | −50.061 | 120.757 | 1.00 | 24.95 | A | C |
| ATOM | 1141 | CG | HIS | 244 | −1.424 | −49.877 | 122.245 | 1.00 | 27.50 | A | C |
| ATOM | 1142 | CD2 | HIS | 244 | −1.780 | −50.711 | 123.252 | 1.00 | 28.29 | A | C |
| ATOM | 1143 | ND1 | HIS | 244 | −1.033 | −48.698 | 122.846 | 1.00 | 28.64 | A | N |
| ATOM | 1144 | CE1 | HIS | 244 | −1.148 | −48.814 | 124.157 | 1.00 | 29.14 | A | C |
| ATOM | 1145 | NE2 | HIS | 244 | −1.600 | −50.025 | 124.430 | 1.00 | 29.46 | A | N |
| ATOM | 1146 | C | HIS | 244 | −3.029 | −48.148 | 120.503 | 1.00 | 21.62 | A | C |
| ATOM | 1147 | O | HIS | 244 | −3.835 | −47.897 | 121.394 | 1.00 | 21.60 | A | O |
| ATOM | 1148 | N | PHE | 245 | −2.355 | −47.211 | 119.844 | 1.00 | 20.43 | A | N |
| ATOM | 1149 | CA | PHE | 245 | −2.560 | −45.793 | 120.106 | 1.00 | 20.28 | A | C |
| ATOM | 1150 | CB | PHE | 245 | −1.700 | −44.935 | 119.173 | 1.00 | 19.48 | A | C |
| ATOM | 1151 | CG | PHE | 245 | −2.127 | −43.495 | 119.121 | 1.00 | 20.46 | A | C |
| ATOM | 1152 | CD1 | PHE | 245 | −1.755 | −42.606 | 120.122 | 1.00 | 20.84 | A | C |
| ATOM | 1153 | CD2 | PHE | 245 | −2.957 | −43.041 | 118.102 | 1.00 | 20.72 | A | C |
| ATOM | 1154 | CE1 | PHE | 245 | −2.206 | −41.284 | 120.113 | 1.00 | 19.78 | A | C |
| ATOM | 1155 | CE2 | PHE | 245 | −3.413 | −41.721 | 118.084 | 1.00 | 21.00 | A | C |
| ATOM | 1156 | CZ | PHE | 245 | −3.034 | −40.844 | 119.096 | 1.00 | 20.53 | A | C |
| ATOM | 1157 | C | PHE | 245 | −4.033 | −45.446 | 119.873 | 1.00 | 19.25 | A | C |
| ATOM | 1158 | O | PHE | 245 | −4.676 | −44.833 | 120.721 | 1.00 | 19.06 | A | O |
| ATOM | 1159 | N | HIS | 246 | −4.564 | −45.844 | 118.722 | 1.00 | 19.01 | A | N |
| ATOM | 1160 | CA | HIS | 246 | −5.954 | −45.546 | 118.400 | 1.00 | 19.09 | A | C |
| ATOM | 1161 | CB | HIS | 246 | −6.268 | −45.962 | 116.953 | 1.00 | 18.50 | A | C |
| ATOM | 1162 | CG | HIS | 246 | −5.954 | −44.898 | 115.944 | 1.00 | 18.48 | A | C |
| ATOM | 1163 | CD2 | HIS | 246 | −4.970 | −44.805 | 115.018 | 1.00 | 18.21 | A | C |
| ATOM | 1164 | ND1 | HIS | 246 | −6.665 | −43.719 | 115.863 | 1.00 | 18.52 | A | N |
| ATOM | 1165 | CE1 | HIS | 246 | −6.130 | −42.945 | 114.935 | 1.00 | 17.66 | A | C |
| ATOM | 1166 | NE2 | HIS | 246 | −5.100 | −43.581 | 114.407 | 1.00 | 18.46 | A | N |
| ATOM | 1167 | C | HIS | 246 | −6.939 | −46.185 | 119.377 | 1.00 | 18.38 | A | C |
| ATOM | 1168 | O | HIS | 246 | −7.899 | −45.550 | 119.801 | 1.00 | 18.13 | A | O |
| ATOM | 1169 | N | GLY | 247 | −6.704 | −47.436 | 119.739 | 1.00 | 19.24 | A | N |
| ATOM | 1170 | CA | GLY | 247 | −7.596 | −48.090 | 120.680 | 1.00 | 19.94 | A | C |
| ATOM | 1171 | C | GLY | 247 | −7.567 | −47.385 | 122.022 | 1.00 | 20.61 | A | C |
| ATOM | 1172 | O | GLY | 247 | −8.615 | −47.103 | 122.602 | 1.00 | 20.87 | A | O |
| ATOM | 1173 | N | THR | 248 | −6.365 | −47.084 | 122.511 | 1.00 | 19.95 | A | N |
| ATOM | 1174 | CA | THR | 248 | −6.211 | −46.413 | 123.793 | 1.00 | 20.64 | A | C |
| ATOM | 1175 | CB | THR | 248 | −4.710 | −46.204 | 124.144 | 1.00 | 21.09 | A | C |
| ATOM | 1176 | OG1 | THR | 248 | −4.030 | −47.467 | 124.116 | 1.00 | 21.47 | A | O |
| ATOM | 1177 | CG2 | THR | 248 | −4.562 | −45.601 | 125.534 | 1.00 | 18.96 | A | C |
| ATOM | 1178 | C | THR | 248 | −6.922 | −45.058 | 123.814 | 1.00 | 20.66 | A | C |
| ATOM | 1179 | O | THR | 248 | −7.642 | −44.736 | 124.766 | 1.00 | 20.22 | A | O |
| ATOM | 1180 | N | LEU | 249 | −6.725 | −44.270 | 122.761 | 1.00 | 21.23 | A | N |
| ATOM | 1181 | CA | LEU | 249 | −7.349 | −42.953 | 122.674 | 1.00 | 21.39 | A | C |
| ATOM | 1182 | CB | LEU | 249 | −6.799 | −42.178 | 121.471 | 1.00 | 21.91 | A | C |
| ATOM | 1183 | CG | LEU | 249 | −7.341 | −40.751 | 121.301 | 1.00 | 22.61 | A | C |
| ATOM | 1184 | CD1 | LEU | 249 | −6.921 | −39.910 | 122.494 | 1.00 | 23.51 | A | C |
| ATOM | 1185 | CD2 | LEU | 249 | −6.818 | −40.133 | 120.014 | 1.00 | 22.87 | A | C |
| ATOM | 1186 | C | LEU | 249 | −8.870 | −43.036 | 122.569 | 1.00 | 20.82 | A | C |
| ATOM | 1187 | O | LEU | 249 | −9.573 | −42.221 | 123.152 | 1.00 | 19.80 | A | O |
| ATOM | 1188 | N | ARG | 250 | −9.374 | −44.023 | 121.828 | 1.00 | 21.20 | A | N |
| ATOM | 1189 | CA | ARG | 250 | −10.816 | −44.185 | 121.644 | 1.00 | 21.98 | A | C |
| ATOM | 1190 | CB | ARG | 250 | −11.100 | −45.245 | 120.573 | 1.00 | 21.92 | A | C |
| ATOM | 1191 | CG | ARG | 250 | −12.563 | −45.341 | 120.158 | 1.00 | 21.63 | A | C |
| ATOM | 1192 | CD | ARG | 250 | −12.994 | −44.086 | 119.412 | 1.00 | 24.25 | A | C |
| ATOM | 1193 | NE | ARG | 250 | −13.967 | −44.395 | 118.368 | 1.00 | 25.98 | A | N |
| ATOM | 1194 | CZ | ARG | 250 | −15.285 | −44.293 | 118.503 | 1.00 | 27.08 | A | C |
| ATOM | 1195 | NH1 | ARG | 250 | −15.813 | −43.874 | 119.644 | 1.00 | 27.73 | A | N |
| ATOM | 1196 | NH2 | ARG | 250 | −16.077 | −44.637 | 117.495 | 1.00 | 27.60 | A | N |
| ATOM | 1197 | C | ARG | 250 | −11.532 | −44.580 | 122.936 | 1.00 | 22.80 | A | C |
| ATOM | 1198 | O | ARG | 250 | −12.645 | −44.126 | 123.194 | 1.00 | 22.12 | A | O |
| ATOM | 1199 | N | LYS | 251 | −10.893 | −45.431 | 123.735 | 1.00 | 23.54 | A | N |
| ATOM | 1200 | CA | LYS | 251 | −11.471 | −45.890 | 124.997 | 1.00 | 24.94 | A | C |
| ATOM | 1201 | CB | LYS | 251 | −10.576 | −46.944 | 125.650 | 1.00 | 25.92 | A | C |
| ATOM | 1202 | CG | LYS | 251 | −10.575 | −48.298 | 124.961 | 1.00 | 29.26 | A | C |
| ATOM | 1203 | CD | LYS | 251 | −9.656 | −49.271 | 125.690 | 1.00 | 30.89 | A | C |
| ATOM | 1204 | CE | LYS | 251 | −9.448 | −50.550 | 124.889 | 1.00 | 33.42 | A | C |
| ATOM | 1205 | NZ | LYS | 251 | −8.346 | −51.387 | 125.462 | 1.00 | 35.04 | A | N |
| ATOM | 1206 | C | LYS | 251 | −11.690 | −44.754 | 125.992 | 1.00 | 25.07 | A | C |
| ATOM | 1207 | O | LYS | 251 | −12.468 | −44.891 | 126.932 | 1.00 | 25.41 | A | O |
| ATOM | 1208 | N | LEU | 252 | −10.996 | −43.639 | 125.793 | 1.00 | 24.90 | A | N |
| ATOM | 1209 | CA | LEU | 252 | −11.138 | −42.499 | 126.685 | 1.00 | 25.36 | A | C |
| ATOM | 1210 | CB | LEU | 252 | −9.953 | −41.549 | 126.503 | 1.00 | 23.70 | A | C |
| ATOM | 1211 | CG | LEU | 252 | −8.638 | −42.172 | 126.985 | 1.00 | 24.00 | A | C |
| ATOM | 1212 | CD1 | LEU | 252 | −7.491 | −41.176 | 126.828 | 1.00 | 22.17 | A | C |
| ATOM | 1213 | CD2 | LEU | 252 | −8.794 | −42.599 | 128.449 | 1.00 | 21.31 | A | C |
| ATOM | 1214 | C | LEU | 252 | −12.457 | −41.748 | 126.504 | 1.00 | 26.07 | A | C |
| ATOM | 1215 | O | LEU | 252 | −12.822 | −40.928 | 127.343 | 1.00 | 25.67 | A | O |
| ATOM | 1216 | N | GLN | 253 | −13.165 | −42.027 | 125.410 | 1.00 | 27.09 | A | N |
| ATOM | 1217 | CA | GLN | 253 | −14.454 | −41.394 | 125.142 | 1.00 | 28.60 | A | C |
| ATOM | 1218 | CB | GLN | 253 | −15.498 | −41.944 | 126.122 | 1.00 | 30.52 | A | C |
| ATOM | 1219 | CG | GLN | 253 | −15.871 | −43.417 | 125.905 | 1.00 | 34.58 | A | C |
| ATOM | 1220 | CD | GLN | 253 | −16.676 | −44.004 | 127.072 | 1.00 | 37.32 | A | C |
| ATOM | 1221 | OE1 | GLN | 253 | −17.588 | −43.365 | 127.602 | 1.00 | 38.93 | A | O |
| ATOM | 1222 | NE2 | GLN | 253 | −16.341 | −45.228 | 127.466 | 1.00 | 38.76 | A | N |
| ATOM | 1223 | C | GLN | 253 | −14.396 | −39.866 | 125.246 | 1.00 | 28.46 | A | C |
| ATOM | 1224 | O | GLN | 253 | −15.168 | −39.256 | 125.986 | 1.00 | 28.32 | A | O |
| ATOM | 1225 | N | LEU | 254 | −13.487 | −39.254 | 124.494 | 1.00 | 27.65 | A | N |
| ATOM | 1226 | CA | LEU | 254 | −13.323 | −37.806 | 124.510 | 1.00 | 27.11 | A | C |
| ATOM | 1227 | CB | LEU | 254 | −12.011 | −37.410 | 123.821 | 1.00 | 25.42 | A | C |
| ATOM | 1228 | CG | LEU | 254 | −10.698 | −37.954 | 124.384 | 1.00 | 24.68 | A | C |
| ATOM | 1229 | CD1 | LEU | 254 | −9.530 | −37.337 | 123.621 | 1.00 | 23.56 | A | C |
| ATOM | 1230 | CD2 | LEU | 254 | −10.599 | −37.627 | 125.867 | 1.00 | 23.28 | A | C |
| ATOM | 1231 | C | LEU | 254 | −14.467 | −37.078 | 123.817 | 1.00 | 27.57 | A | C |
| ATOM | 1232 | O | LEU | 254 | −15.163 | −37.642 | 122.974 | 1.00 | 26.67 | A | O |
| ATOM | 1233 | N | GLN | 255 | −14.648 | −35.814 | 124.176 | 1.00 | 28.10 | A | N |
| ATOM | 1234 | CA | GLN | 255 | −15.676 | −34.990 | 123.562 | 1.00 | 29.49 | A | C |
| ATOM | 1235 | CB | GLN | 255 | −16.329 | −34.095 | 124.616 | 1.00 | 31.52 | A | C |
| ATOM | 1236 | CG | GLN | 255 | −16.861 | −34.865 | 125.810 | 1.00 | 35.22 | A | C |
| ATOM | 1237 | CD | GLN | 255 | −17.594 | −33.978 | 126.791 | 1.00 | 38.55 | A | C |
| ATOM | 1238 | OE1 | GLN | 255 | −17.099 | −32.918 | 127.185 | 1.00 | 39.64 | A | O |
| ATOM | 1239 | NE2 | GLN | 255 | −18.784 | −34.410 | 127.199 | 1.00 | 40.55 | A | N |
| ATOM | 1240 | C | GLN | 255 | −14.978 | −34.145 | 122.496 | 1.00 | 28.88 | A | C |
| ATOM | 1241 | O | GLN | 255 | −13.778 | −33.896 | 122.594 | 1.00 | 28.44 | A | O |
| ATOM | 1242 | N | GLU | 256 | −15.718 | −33.718 | 121.478 | 1.00 | 28.49 | A | N |
| ATOM | 1243 | CA | GLU | 256 | −15.144 | −32.912 | 120.405 | 1.00 | 28.54 | A | C |
| ATOM | 1244 | CB | GLU | 256 | −16.240 | −32.201 | 119.607 | 1.00 | 30.20 | A | C |
| ATOM | 1245 | CG | GLU | 256 | −16.750 | −32.974 | 118.410 | 1.00 | 33.85 | A | C |
| ATOM | 1246 | CD | GLU | 256 | −17.234 | −32.053 | 117.304 | 1.00 | 35.60 | A | C |
| ATOM | 1247 | OE1 | GLU | 256 | −18.123 | −31.214 | 117.567 | 1.00 | 37.88 | A | O |
| ATOM | 1248 | OE2 | GLU | 256 | −16.723 | −32.164 | 116.174 | 1.00 | 35.27 | A | O |
| ATOM | 1249 | C | GLU | 256 | −14.118 | −31.873 | 120.848 | 1.00 | 27.93 | A | C |
| ATOM | 1250 | O | GLU | 256 | −13.018 | −31.823 | 120.319 | 1.00 | 27.24 | A | O |
| ATOM | 1251 | N | PRO | 257 | −14.471 | −31.013 | 121.813 | 1.00 | 28.04 | A | N |
| ATOM | 1252 | CD | PRO | 257 | −15.754 | −30.862 | 122.517 | 1.00 | 28.01 | A | C |
| ATOM | 1253 | CA | PRO | 257 | −13.502 | −30.002 | 122.254 | 1.00 | 27.60 | A | C |
| ATOM | 1254 | CB | PRO | 257 | −14.247 | −29.261 | 123.371 | 1.00 | 28.41 | A | C |
| ATOM | 1255 | CG | PRO | 257 | −15.324 | −30.241 | 123.795 | 1.00 | 29.41 | A | C |
| ATOM | 1256 | C | PRO | 257 | −12.150 | −30.561 | 122.698 | 1.00 | 26.72 | A | C |
| ATOM | 1257 | O | PRO | 257 | −11.111 | −29.957 | 122.436 | 1.00 | 27.19 | A | O |
| ATOM | 1258 | N | GLU | 258 | −12.163 | −31.713 | 123.357 | 1.00 | 25.36 | A | N |
| ATOM | 1259 | CA | GLU | 258 | −10.928 | −32.343 | 123.818 | 1.00 | 24.44 | A | C |
| ATOM | 1260 | CB | GLU | 258 | −11.253 | −33.457 | 124.813 | 1.00 | 24.01 | A | C |
| ATOM | 1261 | CG | GLU | 258 | −12.034 | −32.924 | 126.004 | 1.00 | 26.10 | A | C |
| ATOM | 1262 | CD | GLU | 258 | −12.570 | −34.006 | 126.911 | 1.00 | 26.63 | A | C |
| ATOM | 1263 | OE1 | GLU | 258 | −13.128 | −34.998 | 126.397 | 1.00 | 26.48 | A | O |
| ATOM | 1264 | OE2 | GLU | 258 | −12.446 | −33.853 | 128.143 | 1.00 | 28.74 | A | O |
| ATOM | 1265 | C | GLU | 258 | −10.116 | −32.876 | 122.637 | 1.00 | 23.76 | A | C |
| ATOM | 1266 | O | GLU | 258 | −8.893 | −32.779 | 122.634 | 1.00 | 22.67 | A | O |
| ATOM | 1267 | N | TYR | 259 | −10.796 | −33.427 | 121.634 | 1.00 | 22.12 | A | N |
| ATOM | 1268 | CA | TYR | 259 | −10.108 | −33.924 | 120.446 | 1.00 | 22.11 | A | C |
| ATOM | 1269 | CB | TYR | 259 | −11.091 | −34.596 | 119.485 | 1.00 | 21.08 | A | C |
| ATOM | 1270 | CG | TYR | 259 | −11.261 | −36.087 | 119.674 | 1.00 | 21.43 | A | C |
| ATOM | 1271 | CD1 | TYR | 259 | −10.198 | −36.970 | 119.442 | 1.00 | 21.24 | A | C |
| ATOM | 1272 | CE1 | TYR | 259 | −10.365 | −38.354 | 119.561 | 1.00 | 19.51 | A | C |
| ATOM | 1273 | CD2 | TYR | 259 | −12.493 | −36.622 | 120.036 | 1.00 | 20.98 | A | C |
| ATOM | 1274 | CE2 | TYR | 259 | −12.672 | −38.003 | 120.157 | 1.00 | 21.07 | A | C |
| ATOM | 1275 | CZ | TYR | 259 | −11.601 | −38.860 | 119.914 | 1.00 | 19.86 | A | C |
| ATOM | 1276 | OH | TYR | 259 | −11.788 | −40.218 | 120.006 | 1.00 | 18.11 | A | O |
| ATOM | 1277 | C | TYR | 259 | −9.466 | −32.752 | 119.723 | 1.00 | 22.33 | A | C |
| ATOM | 1278 | O | TYR | 259 | −8.308 | −32.817 | 119.295 | 1.00 | 21.92 | A | O |
| ATOM | 1279 | N | VAL | 260 | −10.229 | −31.677 | 119.575 | 1.00 | 22.03 | A | N |
| ATOM | 1280 | CA | VAL | 260 | −9.727 | −30.516 | 118.877 | 1.00 | 23.22 | A | C |
| ATOM | 1281 | CB | VAL | 260 | −10.856 | −29.512 | 118.626 | 1.00 | 24.49 | A | C |
| ATOM | 1282 | CG1 | VAL | 260 | −10.293 | −28.125 | 118.451 | 1.00 | 26.18 | A | C |
| ATOM | 1283 | CG2 | VAL | 260 | −11.609 | −29.913 | 117.372 | 1.00 | 24.67 | A | C |
| ATOM | 1284 | C | VAL | 260 | −8.557 | −29.849 | 119.590 | 1.00 | 22.97 | A | C |
| ATOM | 1285 | O | VAL | 260 | −7.609 | −29.416 | 118.937 | 1.00 | 22.35 | A | O |
| ATOM | 1286 | N | LEU | 261 | −8.620 | −29.766 | 120.916 | 1.00 | 23.08 | A | N |
| ATOM | 1287 | CA | LEU | 261 | −7.532 | −29.161 | 121.679 | 1.00 | 24.42 | A | C |
| ATOM | 1288 | CB | LEU | 261 | −7.936 | −28.975 | 123.145 | 1.00 | 24.90 | A | C |
| ATOM | 1289 | CG | LEU | 261 | −8.902 | −27.817 | 123.411 | 1.00 | 24.74 | A | C |
| ATOM | 1290 | CD1 | LEU | 261 | −9.489 | −27.932 | 124.808 | 1.00 | 25.84 | A | C |
| ATOM | 1291 | CD2 | LEU | 261 | −8.160 | −26.501 | 123.235 | 1.00 | 24.89 | A | C |
| ATOM | 1292 | C | LEU | 261 | −6.297 | −30.053 | 121.576 | 1.00 | 24.56 | A | C |
| ATOM | 1293 | O | LEU | 261 | −5.170 | −29.560 | 121.469 | 1.00 | 24.62 | A | O |
| ATOM | 1294 | N | LEU | 262 | −6.507 | −31.367 | 121.602 | 1.00 | 24.08 | A | N |
| ATOM | 1295 | CA | LEU | 262 | −5.393 | −32.300 | 121.473 | 1.00 | 24.35 | A | C |
| ATOM | 1296 | CB | LEU | 262 | −5.901 | −33.739 | 121.558 | 1.00 | 25.52 | A | C |
| ATOM | 1297 | CG | LEU | 262 | −4.940 | −34.844 | 122.018 | 1.00 | 26.64 | A | C |
| ATOM | 1298 | CD1 | LEU | 262 | −4.394 | −34.531 | 123.404 | 1.00 | 26.40 | A | C |
| ATOM | 1299 | CD2 | LEU | 262 | −5.693 | −36.172 | 122.044 | 1.00 | 26.95 | A | C |
| ATOM | 1300 | C | LEU | 262 | −4.729 | −32.037 | 120.112 | 1.00 | 23.84 | A | C |
| ATOM | 1301 | O | LEU | 262 | −3.504 | −32.002 | 120.009 | 1.00 | 23.68 | A | O |
| ATOM | 1302 | N | ALA | 263 | −5.535 | −31.833 | 119.071 | 1.00 | 22.30 | A | N |
| ATOM | 1303 | CA | ALA | 263 | −4.987 | −31.551 | 117.749 | 1.00 | 22.57 | A | C |
| ATOM | 1304 | CB | ALA | 263 | −6.102 | −31.473 | 116.710 | 1.00 | 21.76 | A | C |
| ATOM | 1305 | C | ALA | 263 | −4.210 | −30.234 | 117.779 | 1.00 | 22.78 | A | C |
| ATOM | 1306 | O | ALA | 263 | −3.136 | −30.126 | 117.186 | 1.00 | 22.16 | A | O |
| ATOM | 1307 | N | ALA | 264 | −4.762 | −29.239 | 118.470 | 1.00 | 22.57 | A | N |
| ATOM | 1308 | CA | ALA | 264 | −4.123 | −27.934 | 118.586 | 1.00 | 24.00 | A | C |
| ATOM | 1309 | CB | ALA | 264 | −5.043 | −26.965 | 119.332 | 1.00 | 23.05 | A | C |
| ATOM | 1310 | C | ALA | 264 | −2.772 | −28.043 | 119.302 | 1.00 | 24.41 | A | C |
| ATOM | 1311 | O | ALA | 264 | −1.814 | −27.357 | 118.944 | 1.00 | 24.83 | A | O |
| ATOM | 1312 | N | MET | 265 | −2.699 | −28.904 | 120.312 | 1.00 | 25.02 | A | N |
| ATOM | 1313 | CA | MET | 265 | −1.462 | −29.094 | 121.055 | 1.00 | 25.93 | A | C |
| ATOM | 1314 | CB | MET | 265 | −1.720 | −29.938 | 122.306 | 1.00 | 26.79 | A | C |
| ATOM | 1315 | CG | MET | 265 | −2.440 | −29.175 | 123.415 | 1.00 | 27.47 | A | C |
| ATOM | 1316 | SD | MET | 265 | −2.957 | −30.218 | 124.797 | 1.00 | 28.87 | A | S |
| ATOM | 1317 | CE | MET | 265 | −1.384 | −30.422 | 125.669 | 1.00 | 27.94 | A | C |
| ATOM | 1318 | C | MET | 265 | −0.404 | −29.751 | 120.177 | 1.00 | 26.28 | A | C |
| ATOM | 1319 | O | MET | 265 | 0.785 | −29.434 | 120.276 | 1.00 | 26.21 | A | O |
| ATOM | 1320 | N | ALA | 266 | −0.830 | −30.667 | 119.314 | 1.00 | 25.89 | A | N |
| ATOM | 1321 | CA | ALA | 266 | 0.112 | −31.328 | 118.417 | 1.00 | 25.79 | A | C |
| ATOM | 1322 | CB | ALA | 266 | −0.570 | −32.488 | 117.700 | 1.00 | 24.34 | A | C |
| ATOM | 1323 | C | ALA | 266 | 0.616 | −30.295 | 117.402 | 1.00 | 26.44 | A | C |
| ATOM | 1324 | O | ALA | 266 | 1.801 | −30.272 | 117.060 | 1.00 | 25.58 | A | O |
| ATOM | 1325 | N | LEU | 267 | −0.292 | −29.439 | 116.936 | 1.00 | 26.96 | A | N |
| ATOM | 1326 | CA | LEU | 267 | 0.041 | −28.401 | 115.968 | 1.00 | 29.21 | A | C |
| ATOM | 1327 | CB | LEU | 267 | −1.218 | −27.636 | 115.547 | 1.00 | 28.28 | A | C |
| ATOM | 1328 | CG | LEU | 267 | −1.322 | −27.086 | 114.116 | 1.00 | 28.22 | A | C |
| ATOM | 1329 | CD1 | LEU | 267 | −2.153 | −25.815 | 114.151 | 1.00 | 26.62 | A | C |
| ATOM | 1330 | CD2 | LEU | 267 | 0.040 | −26.797 | 113.520 | 1.00 | 27.55 | A | C |
| ATOM | 1331 | C | LEU | 267 | 1.051 | −27.405 | 116.541 | 1.00 | 30.31 | A | C |
| ATOM | 1332 | O | LEU | 267 | 2.114 | −27.195 | 115.966 | 1.00 | 30.73 | A | O |
| ATOM | 1333 | N | PHE | 268 | 0.720 | −26.795 | 117.674 | 1.00 | 31.98 | A | N |
| ATOM | 1334 | CA | PHE | 268 | 1.615 | −25.817 | 118.281 | 1.00 | 34.42 | A | C |
| ATOM | 1335 | CB | PHE | 268 | 0.811 | −24.778 | 119.065 | 1.00 | 33.07 | A | C |
| ATOM | 1336 | CG | PHE | 268 | −0.129 | −23.980 | 118.209 | 1.00 | 33.01 | A | C |
| ATOM | 1337 | CD1 | PHE | 268 | −1.497 | −24.236 | 118.224 | 1.00 | 31.94 | A | C |
| ATOM | 1338 | CD2 | PHE | 268 | 0.357 | −22.995 | 117.356 | 1.00 | 32.71 | A | C |
| ATOM | 1339 | CE1 | PHE | 268 | −2.366 | −23.526 | 117.402 | 1.00 | 31.94 | A | C |
| ATOM | 1340 | CE2 | PHE | 268 | −0.507 | −22.278 | 116.527 | 1.00 | 32.75 | A | C |
| ATOM | 1341 | CZ | PHE | 268 | −1.871 | −22.545 | 116.551 | 1.00 | 32.01 | A | C |
| ATOM | 1342 | C | PHE | 268 | 2.690 | −26.424 | 119.171 | 1.00 | 35.98 | A | C |
| ATOM | 1343 | O | PHE | 268 | 2.635 | −26.328 | 120.393 | 1.00 | 36.42 | A | O |
| ATOM | 1344 | N | SER | 269 | 3.672 | −27.050 | 118.537 | 1.00 | 38.74 | A | N |
| ATOM | 1345 | CA | SER | 269 | 4.785 | −27.669 | 119.244 | 1.00 | 41.31 | A | C |
| ATOM | 1346 | CB | SER | 269 | 5.001 | −29.096 | 118.743 | 1.00 | 41.54 | A | C |
| ATOM | 1347 | OG | SER | 269 | 3.849 | −29.890 | 118.970 | 1.00 | 43.06 | A | O |
| ATOM | 1348 | C | SER | 269 | 6.026 | −26.834 | 118.969 | 1.00 | 42.81 | A | C |
| ATOM | 1349 | O | SER | 269 | 6.505 | −26.782 | 117.840 | 1.00 | 43.23 | A | O |
| ATOM | 1350 | N | PRO | 270 | 6.564 | −26.171 | 120.002 | 1.00 | 44.83 | A | N |
| ATOM | 1351 | CD | PRO | 270 | 6.116 | −26.232 | 121.406 | 1.00 | 44.95 | A | C |
| ATOM | 1352 | CA | PRO | 270 | 7.758 | −25.326 | 119.873 | 1.00 | 46.18 | A | C |
| ATOM | 1353 | CB | PRO | 270 | 7.883 | −24.699 | 121.259 | 1.00 | 45.92 | A | C |
| ATOM | 1354 | CG | PRO | 270 | 7.351 | −25.782 | 122.156 | 1.00 | 45.81 | A | C |
| ATOM | 1355 | C | PRO | 270 | 9.034 | −26.055 | 119.452 | 1.00 | 47.67 | A | C |
| ATOM | 1356 | O | PRO | 270 | 9.967 | −25.437 | 118.935 | 1.00 | 48.27 | A | O |
| ATOM | 1357 | N | ASP | 271 | 9.078 | −27.365 | 119.672 | 1.00 | 48.67 | A | N |
| ATOM | 1358 | CA | ASP | 271 | 10.256 | −28.147 | 119.321 | 1.00 | 49.58 | A | C |
| ATOM | 1359 | CB | ASP | 271 | 10.531 | −29.191 | 120.409 | 1.00 | 50.08 | A | C |
| ATOM | 1360 | CG | ASP | 271 | 9.369 | −30.141 | 120.621 | 1.00 | 50.89 | A | C |
| ATOM | 1361 | OD1 | ASP | 271 | 8.217 | −29.676 | 120.767 | 1.00 | 51.46 | A | O |
| ATOM | 1362 | OD2 | ASP | 271 | 9.611 | −31.362 | 120.658 | 1.00 | 51.73 | A | O |
| ATOM | 1363 | C | ASP | 271 | 10.168 | −28.815 | 117.950 | 1.00 | 50.08 | A | C |
| ATOM | 1364 | O | ASP | 271 | 10.933 | −29.731 | 117.644 | 1.00 | 50.01 | A | O |
| ATOM | 1365 | N | ARG | 272 | 9.238 | −28.351 | 117.124 | 1.00 | 50.50 | A | N |
| ATOM | 1366 | CA | ARG | 272 | 9.082 | −28.895 | 115.784 | 1.00 | 51.11 | A | C |
| ATOM | 1367 | CB | ARG | 272 | 7.824 | −28.330 | 115.126 | 1.00 | 50.35 | A | C |
| ATOM | 1368 | CG | ARG | 272 | 7.023 | −29.341 | 114.325 | 1.00 | 49.50 | A | C |
| ATOM | 1369 | CD | ARG | 272 | 5.919 | −29.977 | 115.161 | 1.00 | 48.07 | A | C |
| ATOM | 1370 | NE | ARG | 272 | 6.151 | −31.394 | 115.405 | 1.00 | 47.04 | A | N |
| ATOM | 1371 | CZ | ARG | 272 | 5.237 | −32.240 | 115.873 | 1.00 | 45.93 | A | C |
| ATOM | 1372 | NH1 | ARG | 272 | 4.011 | −31.822 | 116.154 | 1.00 | 45.21 | A | N |
| ATOM | 1373 | NH2 | ARG | 272 | 5.553 | −33.512 | 116.062 | 1.00 | 44.85 | A | N |
| ATOM | 1374 | C | ARG | 272 | 10.317 | −28.449 | 115.005 | 1.00 | 51.77 | A | C |
| ATOM | 1375 | O | ARG | 272 | 10.674 | −27.271 | 115.015 | 1.00 | 52.46 | A | O |
| ATOM | 1376 | N | PRO | 273 | 10.987 | −29.380 | 114.317 | 1.00 | 52.32 | A | N |
| ATOM | 1377 | CD | PRO | 273 | 10.581 | −30.768 | 114.035 | 1.00 | 52.48 | A | C |
| ATOM | 1378 | CA | PRO | 273 | 12.186 | −29.020 | 113.552 | 1.00 | 52.55 | A | C |
| ATOM | 1379 | CB | PRO | 273 | 12.567 | −30.337 | 112.876 | 1.00 | 52.49 | A | C |
| ATOM | 1380 | CG | PRO | 273 | 11.241 | −31.023 | 112.706 | 1.00 | 52.55 | A | C |
| ATOM | 1381 | C | PRO | 273 | 11.998 | −27.879 | 112.550 | 1.00 | 52.78 | A | C |
| ATOM | 1382 | O | PRO | 273 | 11.186 | −27.978 | 111.628 | 1.00 | 52.86 | A | O |
| ATOM | 1383 | N | GLY | 274 | 12.747 | −26.796 | 112.742 | 1.00 | 52.71 | A | N |
| ATOM | 1384 | CA | GLY | 274 | 12.661 | −25.668 | 111.831 | 1.00 | 52.86 | A | C |
| ATOM | 1385 | C | GLY | 274 | 11.921 | −24.428 | 112.306 | 1.00 | 53.31 | A | C |
| ATOM | 1386 | O | GLY | 274 | 11.825 | −23.448 | 111.567 | 1.00 | 52.74 | A | O |
| ATOM | 1387 | N | VAL | 275 | 11.396 | −24.451 | 113.526 | 1.00 | 53.86 | A | N |
| ATOM | 1388 | CA | VAL | 275 | 10.668 | −23.294 | 114.041 | 1.00 | 54.72 | A | C |
| ATOM | 1389 | CB | VAL | 275 | 9.849 | −23.653 | 115.302 | 1.00 | 54.41 | A | C |
| ATOM | 1390 | CG1 | VAL | 275 | 8.906 | −24.806 | 114.998 | 1.00 | 54.40 | A | C |
| ATOM | 1391 | CG2 | VAL | 275 | 10.781 | −24.010 | 116.448 | 1.00 | 54.73 | A | C |
| ATOM | 1392 | C | VAL | 275 | 11.620 | −22.152 | 114.391 | 1.00 | 55.42 | A | C |
| ATOM | 1393 | O | VAL | 275 | 12.793 | −22.376 | 114.694 | 1.00 | 55.34 | A | O |
| ATOM | 1394 | N | THR | 276 | 11.103 | −20.928 | 114.341 | 1.00 | 56.25 | A | N |
| ATOM | 1395 | CA | THR | 276 | 11.888 | −19.738 | 114.657 | 1.00 | 57.04 | A | C |
| ATOM | 1396 | CB | THR | 276 | 12.019 | −18.806 | 113.428 | 1.00 | 57.12 | A | C |
| ATOM | 1397 | OG1 | THR | 276 | 10.728 | −18.310 | 113.054 | 1.00 | 57.58 | A | O |
| ATOM | 1398 | CG2 | THR | 276 | 12.624 | −19.559 | 112.252 | 1.00 | 56.94 | A | C |
| ATOM | 1399 | C | THR | 276 | 11.249 | −18.958 | 115.810 | 1.00 | 57.44 | A | C |
| ATOM | 1400 | O | THR | 276 | 11.939 | −18.283 | 116.573 | 1.00 | 57.90 | A | O |
| ATOM | 1401 | N | GLN | 277 | 9.928 | −19.058 | 115.929 | 1.00 | 57.58 | A | N |
| ATOM | 1402 | CA | GLN | 277 | 9.183 | −18.383 | 116.987 | 1.00 | 57.56 | A | C |
| ATOM | 1403 | CB | GLN | 277 | 7.827 | −17.912 | 116.453 | 1.00 | 57.96 | A | C |
| ATOM | 1404 | CG | GLN | 277 | 7.476 | −16.472 | 116.777 | 1.00 | 59.08 | A | C |
| ATOM | 1405 | CD | GLN | 277 | 7.893 | −15.506 | 115.683 | 1.00 | 59.41 | A | C |
| ATOM | 1406 | OE1 | GLN | 277 | 9.063 | −15.444 | 115.303 | 1.00 | 60.10 | A | O |
| ATOM | 1407 | NE2 | GLN | 277 | 6.933 | −14.746 | 115.171 | 1.00 | 59.47 | A | N |
| ATOM | 1408 | C | GLN | 277 | 8.953 | −19.379 | 118.125 | 1.00 | 57.42 | A | C |
| ATOM | 1409 | O | GLN | 277 | 7.836 | −19.507 | 118.629 | 1.00 | 57.26 | A | O |
| ATOM | 1410 | N | ARG | 278 | 10.009 | −20.078 | 118.531 | 1.00 | 57.27 | A | N |
| ATOM | 1411 | CA | ARG | 278 | 9.899 | −21.080 | 119.585 | 1.00 | 57.49 | A | C |
| ATOM | 1412 | CB | ARG | 278 | 11.279 | −21.630 | 119.951 | 1.00 | 58.82 | A | C |
| ATOM | 1413 | CG | ARG | 278 | 11.208 | −22.952 | 120.705 | 1.00 | 60.52 | A | C |
| ATOM | 1414 | CD | ARG | 278 | 12.581 | −23.453 | 121.124 | 1.00 | 62.18 | A | C |
| ATOM | 1415 | NE | ARG | 278 | 12.567 | −24.877 | 121.465 | 1.00 | 63.94 | A | N |
| ATOM | 1416 | CZ | ARG | 278 | 11.915 | −25.415 | 122.495 | 1.00 | 64.51 | A | C |
| ATOM | 1417 | NH1 | ARG | 278 | 11.979 | −26.725 | 122.697 | 1.00 | 64.55 | A | N |
| ATOM | 1418 | NH2 | ARG | 278 | 11.209 | −24.656 | 123.328 | 1.00 | 64.96 | A | N |
| ATOM | 1419 | C | ARG | 278 | 9.198 | −20.593 | 120.851 | 1.00 | 56.92 | A | C |
| ATOM | 1420 | O | ARG | 278 | 8.325 | −21.281 | 121.380 | 1.00 | 56.95 | A | O |
| ATOM | 1421 | N | ASP | 279 | 9.578 | −19.419 | 121.344 | 1.00 | 56.06 | A | N |
| ATOM | 1422 | CA | ASP | 279 | 8.956 | −18.883 | 122.551 | 1.00 | 55.37 | A | C |
| ATOM | 1423 | CB | ASP | 279 | 9.646 | −17.588 | 122.990 | 1.00 | 56.68 | A | C |
| ATOM | 1424 | CG | ASP | 279 | 11.084 | −17.809 | 123.419 | 1.00 | 57.63 | A | C |
| ATOM | 1425 | OD1 | ASP | 279 | 11.930 | −18.105 | 122.547 | 1.00 | 58.84 | A | O |
| ATOM | 1426 | OD2 | ASP | 279 | 11.367 | −17.692 | 124.629 | 1.00 | 58.07 | A | O |
| ATOM | 1427 | C | ASP | 279 | 7.470 | −18.618 | 122.338 | 1.00 | 54.14 | A | C |
| ATOM | 1428 | O | ASP | 279 | 6.639 | −19.007 | 123.156 | 1.00 | 53.64 | A | O |
| ATOM | 1429 | N | GLU | 280 | 7.142 | −17.955 | 121.235 | 1.00 | 52.98 | A | N |
| ATOM | 1430 | CA | GLU | 280 | 5.759 | −17.634 | 120.918 | 1.00 | 51.96 | A | C |
| ATOM | 1431 | CB | GLU | 280 | 5.691 | −16.869 | 119.598 | 1.00 | 52.88 | A | C |
| ATOM | 1432 | CG | GLU | 280 | 4.345 | −16.227 | 119.329 | 1.00 | 54.43 | A | C |
| ATOM | 1433 | CD | GLU | 280 | 4.264 | −15.603 | 117.951 | 1.00 | 55.52 | A | C |
| ATOM | 1434 | OE1 | GLU | 280 | 5.204 | −14.875 | 117.570 | 1.00 | 56.15 | A | O |
| ATOM | 1435 | OE2 | GLU | 280 | 3.255 | −15.834 | 117.252 | 1.00 | 55.94 | A | O |
| ATOM | 1436 | C | GLU | 280 | 4.908 | −18.898 | 120.821 | 1.00 | 50.62 | A | C |
| ATOM | 1437 | O | GLU | 280 | 3.869 | −19.007 | 121.470 | 1.00 | 50.58 | A | O |
| ATOM | 1438 | N | ILE | 281 | 5.357 | −19.847 | 120.004 | 1.00 | 49.04 | A | N |
| ATOM | 1439 | CA | ILE | 281 | 4.649 | −21.113 | 119.815 | 1.00 | 47.41 | A | C |
| ATOM | 1440 | CB | ILE | 281 | 5.366 | −21.997 | 118.764 | 1.00 | 46.53 | A | C |
| ATOM | 1441 | CG2 | ILE | 281 | 4.672 | −23.349 | 118.655 | 1.00 | 45.22 | A | C |
| ATOM | 1442 | CG1 | ILE | 281 | 5.378 | −21.282 | 117.408 | 1.00 | 45.56 | A | C |
| ATOM | 1443 | CD1 | ILE | 281 | 6.138 | −22.010 | 116.326 | 1.00 | 45.01 | A | C |
| ATOM | 1444 | C | ILE | 281 | 4.533 | −21.886 | 121.131 | 1.00 | 47.29 | A | C |
| ATOM | 1445 | O | ILE | 281 | 3.525 | −22.549 | 121.383 | 1.00 | 46.81 | A | O |
| ATOM | 1446 | N | ASP | 282 | 5.568 | −21.797 | 121.965 | 1.00 | 46.94 | A | N |
| ATOM | 1447 | CA | ASP | 282 | 5.574 | −22.471 | 123.260 | 1.00 | 46.55 | A | C |
| ATOM | 1448 | CB | ASP | 282 | 6.946 | −22.311 | 123.926 | 1.00 | 47.68 | A | C |
| ATOM | 1449 | CG | ASP | 282 | 7.098 | −23.166 | 125.170 | 1.00 | 48.85 | A | C |
| ATOM | 1450 | OD1 | ASP | 282 | 6.921 | −24.402 | 125.082 | 1.00 | 49.57 | A | O |
| ATOM | 1451 | OD2 | ASP | 282 | 7.402 | −22.602 | 126.242 | 1.00 | 49.92 | A | O |
| ATOM | 1452 | C | ASP | 282 | 4.470 | −21.876 | 124.144 | 1.00 | 45.75 | A | C |
| ATOM | 1453 | O | ASP | 282 | 3.847 | −22.583 | 124.934 | 1.00 | 45.36 | A | O |
| ATOM | 1454 | N | GLN | 283 | 4.226 | −20.576 | 124.000 | 1.00 | 45.11 | A | N |
| ATOM | 1455 | CA | GLN | 283 | 3.181 | −19.904 | 124.770 | 1.00 | 45.13 | A | C |
| ATOM | 1456 | CB | GLN | 283 | 3.256 | −18.387 | 124.563 | 1.00 | 46.75 | A | C |
| ATOM | 1457 | CG | GLN | 283 | 2.563 | −17.534 | 125.637 | 1.00 | 50.09 | A | C |
| ATOM | 1458 | CD | GLN | 283 | 1.092 | −17.879 | 125.849 | 1.00 | 52.42 | A | C |
| ATOM | 1459 | OE1 | GLN | 283 | 0.754 | −18.757 | 126.649 | 1.00 | 54.11 | A | O |
| ATOM | 1460 | NE2 | GLN | 283 | 0.210 | −17.189 | 125.129 | 1.00 | 53.63 | A | N |
| ATOM | 1461 | C | GLN | 283 | 1.821 | −20.423 | 124.292 | 1.00 | 43.58 | A | C |
| ATOM | 1462 | O | GLN | 283 | 0.929 | −20.680 | 125.099 | 1.00 | 43.49 | A | O |
| ATOM | 1463 | N | LEU | 284 | 1.664 | −20.573 | 122.979 | 1.00 | 42.02 | A | N |
| ATOM | 1464 | CA | LEU | 284 | 0.409 | −21.079 | 122.428 | 1.00 | 40.90 | A | C |
| ATOM | 1465 | CB | LEU | 284 | 0.438 | −21.086 | 120.892 | 1.00 | 40.60 | A | C |
| ATOM | 1466 | CG | LEU | 284 | 0.221 | −19.783 | 120.110 | 1.00 | 40.12 | A | C |
| ATOM | 1467 | CD1 | LEU | 284 | −0.648 | −18.833 | 120.918 | 1.00 | 39.50 | A | C |
| ATOM | 1468 | CD2 | LEU | 284 | 1.551 | −19.146 | 119.789 | 1.00 | 40.64 | A | C |
| ATOM | 1469 | C | LEU | 284 | 0.140 | −22.491 | 122.934 | 1.00 | 39.59 | A | C |
| ATOM | 1470 | O | LEU | 284 | −0.986 | −22.825 | 123.292 | 1.00 | 39.24 | A | O |
| ATOM | 1471 | N | GLN | 285 | 1.180 | −23.318 | 122.950 | 1.00 | 39.22 | A | N |
| ATOM | 1472 | CA | GLN | 285 | 1.070 | −24.688 | 123.433 | 1.00 | 38.77 | A | C |
| ATOM | 1473 | CB | GLN | 285 | 2.454 | −25.344 | 123.452 | 1.00 | 39.75 | A | C |
| ATOM | 1474 | CG | GLN | 285 | 2.497 | −26.740 | 124.049 | 1.00 | 42.43 | A | C |
| ATOM | 1475 | CD | GLN | 285 | 1.934 | −27.788 | 123.117 | 1.00 | 43.45 | A | C |
| ATOM | 1476 | OE1 | GLN | 285 | 0.771 | −27.728 | 122.730 | 1.00 | 45.03 | A | O |
| ATOM | 1477 | NE2 | GLN | 285 | 2.763 | −28.757 | 122.747 | 1.00 | 44.86 | A | N |
| ATOM | 1478 | C | GLN | 285 | 0.487 | −24.653 | 124.843 | 1.00 | 37.86 | A | C |
| ATOM | 1479 | O | GLN | 285 | −0.501 | −25.323 | 125.138 | 1.00 | 37.02 | A | O |
| ATOM | 1480 | N | GLU | 286 | 1.096 | −23.851 | 125.710 | 1.00 | 37.54 | A | N |
| ATOM | 1481 | CA | GLU | 286 | 0.628 | −23.736 | 127.085 | 1.00 | 37.97 | A | C |
| ATOM | 1482 | CB | GLU | 286 | 1.526 | −22.794 | 127.884 | 1.00 | 39.37 | A | C |
| ATOM | 1483 | CG | GLU | 286 | 2.269 | −23.502 | 128.993 | 1.00 | 42.37 | A | C |
| ATOM | 1484 | CD | GLU | 286 | 1.361 | −24.415 | 129.792 | 1.00 | 44.10 | A | C |
| ATOM | 1485 | OE1 | GLU | 286 | 0.394 | −23.910 | 130.402 | 1.00 | 45.20 | A | O |
| ATOM | 1486 | OE2 | GLU | 286 | 1.612 | −25.640 | 129.803 | 1.00 | 46.83 | A | O |
| ATOM | 1487 | C | GLU | 286 | −0.808 | −23.257 | 127.167 | 1.00 | 36.84 | A | C |
| ATOM | 1488 | O | GLU | 286 | −1.552 | −23.668 | 128.047 | 1.00 | 36.65 | A | O |
| ATOM | 1489 | N | GLU | 287 | −1.187 | −22.376 | 126.251 | 1.00 | 37.13 | A | N |
| ATOM | 1490 | CA | GLU | 287 | −2.541 | −21.848 | 126.203 | 1.00 | 37.20 | A | C |
| ATOM | 1491 | CB | GLU | 287 | −2.625 | −20.761 | 125.128 | 1.00 | 39.14 | A | C |
| ATOM | 1492 | CG | GLU | 287 | −3.881 | −19.906 | 125.181 | 1.00 | 42.57 | A | C |
| ATOM | 1493 | CD | GLU | 287 | −3.893 | −18.822 | 124.108 | 1.00 | 44.54 | A | C |
| ATOM | 1494 | OE1 | GLU | 287 | −2.923 | −18.031 | 124.049 | 1.00 | 45.67 | A | O |
| ATOM | 1495 | OE2 | GLU | 287 | −4.873 | −18.762 | 123.330 | 1.00 | 45.07 | A | O |
| ATOM | 1496 | C | GLU | 287 | −3.517 | −22.987 | 125.885 | 1.00 | 36.19 | A | C |
| ATOM | 1497 | O | GLU | 287 | −4.565 | −23.109 | 126.514 | 1.00 | 35.33 | A | O |
| ATOM | 1498 | N | MET | 288 | −3.161 | −23.819 | 124.907 | 1.00 | 35.38 | A | N |
| ATOM | 1499 | CA | MET | 288 | −3.998 | −24.950 | 124.509 | 1.00 | 34.70 | A | C |
| ATOM | 1500 | CB | MET | 288 | −3.384 | −25.689 | 123.313 | 1.00 | 35.45 | A | C |
| ATOM | 1501 | CG | MET | 288 | −3.064 | −24.841 | 122.100 | 1.00 | 36.49 | A | C |
| ATOM | 1502 | SD | MET | 288 | −4.518 | −24.110 | 121.347 | 1.00 | 37.77 | A | S |
| ATOM | 1503 | CE | MET | 288 | −4.196 | −22.370 | 121.637 | 1.00 | 38.91 | A | C |
| ATOM | 1504 | C | MET | 288 | −4.108 | −25.930 | 125.671 | 1.00 | 33.74 | A | C |
| ATOM | 1505 | O | MET | 288 | −5.202 | −26.379 | 126.025 | 1.00 | 33.02 | A | O |
| ATOM | 1506 | N | ALA | 289 | −2.956 | −26.260 | 126.253 | 1.00 | 33.19 | A | N |
| ATOM | 1507 | CA | ALA | 289 | −2.880 | −27.195 | 127.370 | 1.00 | 32.47 | A | C |
| ATOM | 1508 | CB | ALA | 289 | −1.431 | −27.340 | 127.826 | 1.00 | 31.87 | A | C |
| ATOM | 1509 | C | ALA | 289 | −3.760 | −26.772 | 128.541 | 1.00 | 32.22 | A | C |
| ATOM | 1510 | O | ALA | 289 | −4.537 | −27.575 | 129.066 | 1.00 | 31.24 | A | O |
| ATOM | 1511 | N | LEU | 290 | −3.634 | −25.512 | 128.948 | 1.00 | 32.24 | A | N |
| ATOM | 1512 | CA | LEU | 290 | −4.420 | −24.985 | 130.055 | 1.00 | 32.90 | A | C |
| ATOM | 1513 | CB | LEU | 290 | −4.009 | −23.543 | 130.348 | 1.00 | 34.69 | A | C |
| ATOM | 1514 | CG | LEU | 290 | −2.627 | −23.383 | 130.975 | 1.00 | 35.82 | A | C |
| ATOM | 1515 | CD1 | LEU | 290 | −2.309 | −21.900 | 131.153 | 1.00 | 36.30 | A | C |
| ATOM | 1516 | CD2 | LEU | 290 | −2.601 | −24.111 | 132.314 | 1.00 | 36.19 | A | C |
| ATOM | 1517 | C | LEU | 290 | −5.910 | −25.050 | 129.761 | 1.00 | 32.17 | A | C |
| ATOM | 1518 | O | LEU | 290 | −6.707 | −25.393 | 130.628 | 1.00 | 32.35 | A | O |
| ATOM | 1519 | N | THR | 291 | −6.283 | −24.713 | 128.533 | 1.00 | 31.75 | A | N |
| ATOM | 1520 | CA | THR | 291 | −7.680 | −24.761 | 128.124 | 1.00 | 31.71 | A | C |
| ATOM | 1521 | CB | THR | 291 | −7.837 | −24.298 | 126.662 | 1.00 | 32.01 | A | C |
| ATOM | 1522 | OG1 | THR | 291 | −7.204 | −23.022 | 126.497 | 1.00 | 32.68 | A | O |
| ATOM | 1523 | CG2 | THR | 291 | −9.312 | −24.179 | 126.291 | 1.00 | 31.69 | A | C |
| ATOM | 1524 | C | THR | 291 | −8.192 | −26.199 | 128.252 | 1.00 | 31.72 | A | C |
| ATOM | 1525 | O | THR | 291 | −9.289 | −26.434 | 128.760 | 1.00 | 31.36 | A | O |
| ATOM | 1526 | N | LEU | 292 | −7.392 | −27.159 | 127.792 | 1.00 | 31.96 | A | N |
| ATOM | 1527 | CA | LEU | 292 | −7.772 | −28.567 | 127.864 | 1.00 | 32.82 | A | C |
| ATOM | 1528 | CB | LEU | 292 | −6.672 | −29.464 | 127.278 | 1.00 | 31.84 | A | C |
| ATOM | 1529 | CG | LEU | 292 | −7.088 | −30.797 | 126.636 | 1.00 | 32.12 | A | C |
| ATOM | 1530 | CD1 | LEU | 292 | −5.901 | −31.753 | 126.649 | 1.00 | 30.55 | A | C |
| ATOM | 1531 | CD2 | LEU | 292 | −8.265 | −31.413 | 127.367 | 1.00 | 31.13 | A | C |
| ATOM | 1532 | C | LEU | 292 | −8.005 | −28.960 | 129.318 | 1.00 | 33.29 | A | C |
| ATOM | 1533 | O | LEU | 292 | −9.011 | −29.582 | 129.643 | 1.00 | 32.92 | A | O |
| ATOM | 1534 | N | GLN | 293 | −7.061 | −28.606 | 130.185 | 1.00 | 34.69 | A | N |
| ATOM | 1535 | CA | GLN | 293 | −7.170 | −28.920 | 131.603 | 1.00 | 36.33 | A | C |
| ATOM | 1536 | CB | GLN | 293 | −5.973 | −28.369 | 132.368 | 1.00 | 37.25 | A | C |
| ATOM | 1537 | CG | GLN | 293 | −4.664 | −29.038 | 132.047 | 1.00 | 38.64 | A | C |
| ATOM | 1538 | CD | GLN | 293 | −3.551 | −28.560 | 132.948 | 1.00 | 39.42 | A | C |
| ATOM | 1539 | OE1 | GLN | 293 | −3.615 | −28.720 | 134.169 | 1.00 | 40.32 | A | O |
| ATOM | 1540 | NE2 | GLN | 293 | −2.521 | −27.966 | 132.355 | 1.00 | 39.89 | A | N |
| ATOM | 1541 | C | GLN | 293 | −8.444 | −28.337 | 132.194 | 1.00 | 37.50 | A | C |
| ATOM | 1542 | O | GLN | 293 | −9.207 | −29.043 | 132.852 | 1.00 | 37.40 | A | O |
| ATOM | 1543 | N | SER | 294 | −8.661 | −27.044 | 131.969 | 1.00 | 38.47 | A | N |
| ATOM | 1544 | CA | SER | 294 | −9.850 | −26.367 | 132.480 | 1.00 | 39.65 | A | C |
| ATOM | 1545 | CB | SER | 294 | −9.890 | −24.916 | 131.994 | 1.00 | 39.69 | A | C |
| ATOM | 1546 | OG | SER | 294 | −8.707 | −24.222 | 132.355 | 1.00 | 40.48 | A | O |
| ATOM | 1547 | C | SER | 294 | −11.092 | −27.098 | 131.996 | 1.00 | 40.07 | A | C |
| ATOM | 1548 | O | SER | 294 | −12.013 | −27.358 | 132.770 | 1.00 | 40.28 | A | O |
| ATOM | 1549 | N | TYR | 295 | −11.106 | −27.439 | 130.712 | 1.00 | 40.29 | A | N |
| ATOM | 1550 | CA | TYR | 295 | −12.239 | −28.135 | 130.129 | 1.00 | 41.02 | A | C |
| ATOM | 1551 | CB | TYR | 295 | −12.006 | −28.362 | 128.633 | 1.00 | 40.48 | A | C |
| ATOM | 1552 | CG | TYR | 295 | −13.219 | −28.921 | 127.941 | 1.00 | 40.03 | A | C |
| ATOM | 1553 | CD1 | TYR | 295 | −13.535 | −30.275 | 128.033 | 1.00 | 39.72 | A | C |
| ATOM | 1554 | CE1 | TYR | 295 | −14.708 | −30.775 | 127.483 | 1.00 | 40.15 | A | C |
| ATOM | 1555 | CD2 | TYR | 295 | −14.106 | −28.080 | 127.271 | 1.00 | 39.70 | A | C |
| ATOM | 1556 | CE2 | TYR | 295 | −15.279 | −28.568 | 126.722 | 1.00 | 39.35 | A | C |
| ATOM | 1557 | CZ | TYR | 295 | −15.574 | −29.915 | 126.834 | 1.00 | 40.12 | A | C |
| ATOM | 1558 | OH | TYR | 295 | −16.748 | −30.399 | 126.308 | 1.00 | 41.32 | A | O |
| ATOM | 1559 | C | TYR | 295 | −12.507 | −29.465 | 130.824 | 1.00 | 41.79 | A | C |
| ATOM | 1560 | O | TYR | 295 | −13.649 | −29.785 | 131.146 | 1.00 | 42.03 | A | O |
| ATOM | 1561 | N | ILE | 296 | −11.455 | −30.241 | 131.051 | 1.00 | 42.99 | A | N |
| ATOM | 1562 | CA | ILE | 296 | −11.591 | −31.532 | 131.711 | 1.00 | 44.15 | A | C |
| ATOM | 1563 | CB | ILE | 296 | −10.250 | −32.290 | 131.718 | 1.00 | 43.14 | A | C |
| ATOM | 1564 | CG2 | ILE | 296 | −10.377 | −33.564 | 132.540 | 1.00 | 42.45 | A | C |
| ATOM | 1565 | CG1 | ILE | 296 | −9.823 | −32.597 | 130.279 | 1.00 | 42.93 | A | C |
| ATOM | 1566 | CD1 | ILE | 296 | −8.468 | −33.258 | 130.163 | 1.00 | 41.86 | A | C |
| ATOM | 1567 | C | ILE | 296 | −12.080 | −31.365 | 133.150 | 1.00 | 45.76 | A | C |
| ATOM | 1568 | O | ILE | 296 | −12.958 | −32.099 | 133.604 | 1.00 | 45.29 | A | O |
| ATOM | 1569 | N | LYS | 297 | −11.508 | −30.401 | 133.866 | 1.00 | 48.10 | A | N |
| ATOM | 1570 | CA | LYS | 297 | −11.901 | −30.141 | 135.248 | 1.00 | 51.07 | A | C |
| ATOM | 1571 | CB | LYS | 297 | −11.079 | −28.985 | 135.828 | 1.00 | 51.11 | A | C |
| ATOM | 1572 | CG | LYS | 297 | −9.628 | −29.359 | 136.112 | 1.00 | 52.35 | A | C |
| ATOM | 1573 | CD | LYS | 297 | −8.831 | −28.200 | 136.696 | 1.00 | 53.18 | A | C |
| ATOM | 1574 | CE | LYS | 297 | −7.404 | −28.635 | 137.020 | 1.00 | 54.08 | A | C |
| ATOM | 1575 | NZ | LYS | 297 | −6.565 | −27.527 | 137.567 | 1.00 | 54.48 | A | N |
| ATOM | 1576 | C | LYS | 297 | −13.392 | −29.835 | 135.362 | 1.00 | 53.02 | A | C |
| ATOM | 1577 | O | LYS | 297 | −14.038 | −30.238 | 136.328 | 1.00 | 53.38 | A | O |
| ATOM | 1578 | N | GLY | 298 | −13.938 | −29.132 | 134.374 | 1.00 | 55.42 | A | N |
| ATOM | 1579 | CA | GLY | 298 | −15.353 | −28.810 | 134.397 | 1.00 | 58.64 | A | C |
| ATOM | 1580 | C | GLY | 298 | −16.214 | −29.936 | 133.854 | 1.00 | 61.18 | A | C |
| ATOM | 1581 | O | GLY | 298 | −17.337 | −29.704 | 133.409 | 1.00 | 61.26 | A | O |
| ATOM | 1582 | N | GLN | 299 | −15.684 | −31.156 | 133.895 | 1.00 | 63.92 | A | N |
| ATOM | 1583 | CA | GLN | 299 | −16.379 | −32.344 | 133.405 | 1.00 | 66.98 | A | C |
| ATOM | 1584 | CB | GLN | 299 | −15.633 | −33.613 | 133.853 | 1.00 | 67.13 | A | C |
| ATOM | 1585 | CG | GLN | 299 | −15.230 | −33.657 | 135.333 | 1.00 | 67.29 | A | C |
| ATOM | 1586 | CD | GLN | 299 | −16.415 | −33.750 | 136.282 | 1.00 | 67.70 | A | C |
| ATOM | 1587 | OE1 | GLN | 299 | −17.196 | −34.701 | 136.230 | 1.00 | 67.82 | A | O |
| ATOM | 1588 | NE2 | GLN | 299 | −16.551 | −32.760 | 137.161 | 1.00 | 67.75 | A | N |
| ATOM | 1589 | C | GLN | 299 | −17.835 | −32.416 | 133.850 | 1.00 | 68.93 | A | C |
| ATOM | 1590 | O | GLN | 299 | −18.228 | −31.783 | 134.831 | 1.00 | 69.45 | A | O |
| ATOM | 1591 | N | GLN | 300 | −18.634 | −33.186 | 133.116 | 1.00 | 71.11 | A | N |
| ATOM | 1592 | CA | GLN | 300 | −20.045 | −33.340 | 133.446 | 1.00 | 73.14 | A | C |
| ATOM | 1593 | CB | GLN | 300 | −20.819 | −33.903 | 132.250 | 1.00 | 73.51 | A | C |
| ATOM | 1594 | CG | GLN | 300 | −21.287 | −32.844 | 131.267 | 1.00 | 74.36 | A | C |
| ATOM | 1595 | CD | GLN | 300 | −22.243 | −31.847 | 131.902 | 1.00 | 75.03 | A | C |
| ATOM | 1596 | OE1 | GLN | 300 | −21.883 | −31.131 | 132.842 | 1.00 | 75.24 | A | O |
| ATOM | 1597 | NE2 | GLN | 300 | −23.471 | −31.797 | 131.392 | 1.00 | 75.25 | A | N |
| ATOM | 1598 | C | GLN | 300 | −20.246 | −34.235 | 134.661 | 1.00 | 74.34 | A | C |
| ATOM | 1599 | O | GLN | 300 | −20.722 | −33.777 | 135.700 | 1.00 | 74.59 | A | O |
| ATOM | 1600 | N | ARG | 301 | −19.883 | −35.509 | 134.543 | 1.00 | 75.65 | A | N |
| ATOM | 1601 | CA | ARG | 301 | −20.049 | −36.419 | 135.667 | 1.00 | 76.68 | A | C |
| ATOM | 1602 | CB | ARG | 301 | −21.478 | −36.985 | 135.677 | 1.00 | 77.20 | A | C |
| ATOM | 1603 | CG | ARG | 301 | −21.959 | −37.404 | 137.063 | 1.00 | 78.13 | A | C |
| ATOM | 1604 | CD | ARG | 301 | −23.132 | −38.365 | 137.011 | 1.00 | 78.94 | A | C |
| ATOM | 1605 | NE | ARG | 301 | −23.446 | −38.876 | 138.344 | 1.00 | 79.59 | A | N |
| ATOM | 1606 | CZ | ARG | 301 | −24.354 | −39.813 | 138.602 | 1.00 | 79.96 | A | C |
| ATOM | 1607 | NH1 | ARG | 301 | −24.557 | −40.203 | 139.854 | 1.00 | 80.06 | A | N |
| ATOM | 1608 | NH2 | ARG | 301 | −25.057 | −40.362 | 137.617 | 1.00 | 79.96 | A | N |
| ATOM | 1609 | C | ARG | 301 | −19.036 | −37.568 | 135.678 | 1.00 | 76.98 | A | C |
| ATOM | 1610 | O | ARG | 301 | −18.188 | −37.686 | 134.788 | 1.00 | 76.77 | A | O |
| ATOM | 1611 | N | ARG | 302 | −19.141 | −38.399 | 136.713 | 1.00 | 77.23 | A | N |
| ATOM | 1612 | CA | ARG | 302 | −18.288 | −39.563 | 136.920 | 1.00 | 77.33 | A | C |
| ATOM | 1613 | CB | ARG | 302 | −18.689 | −40.250 | 138.232 | 1.00 | 77.94 | A | C |
| ATOM | 1614 | CG | ARG | 302 | −18.767 | −39.306 | 139.425 | 1.00 | 79.00 | A | C |
| ATOM | 1615 | CD | ARG | 302 | −19.352 | −39.993 | 140.647 | 1.00 | 80.06 | A | C |
| ATOM | 1616 | NE | ARG | 302 | −19.619 | −39.045 | 141.727 | 1.00 | 80.81 | A | N |
| ATOM | 1617 | CZ | ARG | 302 | −20.120 | −39.375 | 142.915 | 1.00 | 81.28 | A | C |
| ATOM | 1618 | NH1 | ARG | 302 | −20.414 | −40.640 | 143.190 | 1.00 | 81.53 | A | N |
| ATOM | 1619 | NH2 | ARG | 302 | −20.329 | −38.437 | 143.830 | 1.00 | 81.32 | A | N |
| ATOM | 1620 | C | ARG | 302 | −18.432 | −40.541 | 135.748 | 1.00 | 76.86 | A | C |
| ATOM | 1621 | O | ARG | 302 | −19.245 | −40.326 | 134.848 | 1.00 | 77.13 | A | O |
| ATOM | 1622 | N | PRO | 303 | −17.641 | −41.627 | 135.740 | 1.00 | 76.19 | A | N |
| ATOM | 1623 | CD | PRO | 303 | −17.651 | −42.598 | 134.632 | 1.00 | 76.11 | A | C |
| ATOM | 1624 | CA | PRO | 303 | −16.638 | −41.999 | 136.747 | 1.00 | 75.35 | A | C |
| ATOM | 1625 | CB | PRO | 303 | −16.150 | −43.362 | 136.257 | 1.00 | 75.81 | A | C |
| ATOM | 1626 | CG | PRO | 303 | −16.298 | −43.254 | 134.770 | 1.00 | 75.99 | A | C |
| ATOM | 1627 | C | PRO | 303 | −15.502 | −40.983 | 136.888 | 1.00 | 74.26 | A | C |
| ATOM | 1628 | O | PRO | 303 | −14.879 | −40.586 | 135.901 | 1.00 | 74.49 | A | O |
| ATOM | 1629 | N | ARG | 304 | −15.251 | −40.562 | 138.125 | 1.00 | 72.62 | A | N |
| ATOM | 1630 | CA | ARG | 304 | −14.193 | −39.600 | 138.422 | 1.00 | 70.74 | A | C |
| ATOM | 1631 | CB | ARG | 304 | −13.927 | −39.545 | 139.932 | 1.00 | 71.66 | A | C |
| ATOM | 1632 | CG | ARG | 304 | −13.223 | −40.787 | 140.498 | 1.00 | 72.36 | A | C |
| ATOM | 1633 | CD | ARG | 304 | −14.164 | −41.984 | 140.613 | 1.00 | 73.27 | A | C |
| ATOM | 1634 | NE | ARG | 304 | −13.466 | −43.229 | 140.936 | 1.00 | 73.97 | A | N |
| ATOM | 1635 | CZ | ARG | 304 | −12.741 | −43.940 | 140.075 | 1.00 | 74.21 | A | C |
| ATOM | 1636 | NH1 | ARG | 304 | −12.601 | −43.543 | 138.817 | 1.00 | 74.26 | A | N |
| ATOM | 1637 | NH2 | ARG | 304 | −12.154 | −45.060 | 140.473 | 1.00 | 74.59 | A | N |
| ATOM | 1638 | C | ARG | 304 | −12.909 | −40.014 | 137.716 | 1.00 | 68.87 | A | C |
| ATOM | 1639 | O | ARG | 304 | −12.746 | −41.179 | 137.351 | 1.00 | 68.86 | A | O |
| ATOM | 1640 | N | ASP | 305 | −11.995 | −39.066 | 137.526 | 1.00 | 66.37 | A | N |
| ATOM | 1641 | CA | ASP | 305 | −10.727 | −39.378 | 136.875 | 1.00 | 62.94 | A | C |
| ATOM | 1642 | CB | ASP | 305 | −10.978 | −40.055 | 135.525 | 1.00 | 63.29 | A | C |
| ATOM | 1643 | CG | ASP | 305 | −9.704 | −40.566 | 134.887 | 1.00 | 63.63 | A | C |
| ATOM | 1644 | OD1 | ASP | 305 | −8.944 | −41.283 | 135.570 | 1.00 | 63.64 | A | O |
| ATOM | 1645 | OD2 | ASP | 305 | −9.465 | −40.259 | 133.701 | 1.00 | 64.48 | A | O |
| ATOM | 1646 | C | ASP | 305 | −9.804 | −38.183 | 136.670 | 1.00 | 60.10 | A | C |
| ATOM | 1647 | O | ASP | 305 | −9.951 | −37.428 | 135.704 | 1.00 | 59.69 | A | O |
| ATOM | 1648 | N | ARG | 306 | −8.855 | −38.005 | 137.584 | 1.00 | 56.47 | A | N |
| ATOM | 1649 | CA | ARG | 306 | −7.896 | −36.918 | 137.437 | 1.00 | 52.72 | A | C |
| ATOM | 1650 | CB | ARG | 306 | −7.376 | −36.424 | 138.792 | 1.00 | 54.11 | A | C |
| ATOM | 1651 | CG | ARG | 306 | −6.551 | −35.137 | 138.659 | 1.00 | 55.62 | A | C |
| ATOM | 1652 | CD | ARG | 306 | −5.264 | −35.152 | 139.480 | 1.00 | 57.19 | A | C |
| ATOM | 1653 | NE | ARG | 306 | −5.384 | −34.423 | 140.743 | 1.00 | 58.88 | A | N |
| ATOM | 1654 | CZ | ARG | 306 | −4.358 | −34.121 | 141.537 | 1.00 | 59.27 | A | C |
| ATOM | 1655 | NH1 | ARG | 306 | −3.125 | −34.487 | 141.204 | 1.00 | 59.61 | A | N |
| ATOM | 1656 | NH2 | ARG | 306 | −4.561 | −33.443 | 142.662 | 1.00 | 59.29 | A | N |
| ATOM | 1657 | C | ARG | 306 | −6.721 | −37.437 | 136.612 | 1.00 | 49.02 | A | C |
| ATOM | 1658 | O | ARG | 306 | −5.719 | −36.750 | 136.448 | 1.00 | 49.06 | A | O |
| ATOM | 1659 | N | PHE | 307 | −6.857 | −38.659 | 136.101 | 1.00 | 44.87 | A | N |
| ATOM | 1660 | CA | PHE | 307 | −5.828 | −39.299 | 135.288 | 1.00 | 40.64 | A | C |
| ATOM | 1661 | CB | PHE | 307 | −5.889 | −40.819 | 135.477 | 1.00 | 39.73 | A | C |
| ATOM | 1662 | CG | PHE | 307 | −5.603 | −41.275 | 136.880 | 1.00 | 39.02 | A | C |
| ATOM | 1663 | CD1 | PHE | 307 | −4.295 | −41.358 | 137.349 | 1.00 | 38.54 | A | C |
| ATOM | 1664 | CD2 | PHE | 307 | −6.644 | −41.615 | 137.738 | 1.00 | 38.66 | A | C |
| ATOM | 1665 | CE1 | PHE | 307 | −4.026 | −41.774 | 138.650 | 1.00 | 38.12 | A | C |
| ATOM | 1666 | CE2 | PHE | 307 | −6.385 | −42.032 | 139.044 | 1.00 | 39.04 | A | C |
| ATOM | 1667 | CZ | PHE | 307 | −5.072 | −42.112 | 139.499 | 1.00 | 38.17 | A | C |
| ATOM | 1668 | C | PHE | 307 | −5.976 | −38.982 | 133.796 | 1.00 | 38.25 | A | C |
| ATOM | 1669 | O | PHE | 307 | −5.036 | −39.160 | 133.026 | 1.00 | 36.74 | A | O |
| ATOM | 1670 | N | LEU | 308 | −7.150 | −38.504 | 133.391 | 1.00 | 35.76 | A | N |
| ATOM | 1671 | CA | LEU | 308 | −7.397 | −38.212 | 131.984 | 1.00 | 33.41 | A | C |
| ATOM | 1672 | CB | LEU | 308 | −8.819 | −37.672 | 131.792 | 1.00 | 33.07 | A | C |
| ATOM | 1673 | CG | LEU | 308 | −9.360 | −37.618 | 130.351 | 1.00 | 33.17 | A | C |
| ATOM | 1674 | CD1 | LEU | 308 | −8.779 | −36.444 | 129.613 | 1.00 | 34.19 | A | C |
| ATOM | 1675 | CD2 | LEU | 308 | −9.036 | −38.913 | 129.623 | 1.00 | 31.34 | A | C |
| ATOM | 1676 | C | LEU | 308 | −6.389 | −37.268 | 131.337 | 1.00 | 31.73 | A | C |
| ATOM | 1677 | O | LEU | 308 | −5.809 | −37.601 | 130.306 | 1.00 | 30.35 | A | O |
| ATOM | 1678 | N | TYR | 309 | −6.176 | −36.097 | 131.928 | 1.00 | 29.96 | A | N |
| ATOM | 1679 | CA | TYR | 309 | −5.236 | −35.149 | 131.346 | 1.00 | 28.55 | A | C |
| ATOM | 1680 | CB | TYR | 309 | −5.156 | −33.865 | 132.171 | 1.00 | 28.94 | A | C |
| ATOM | 1681 | CG | TYR | 309 | −4.250 | −32.828 | 131.543 | 1.00 | 29.27 | A | C |
| ATOM | 1682 | CD1 | TYR | 309 | −4.567 | −32.250 | 130.312 | 1.00 | 29.24 | A | C |
| ATOM | 1683 | CE1 | TYR | 309 | −3.719 | −31.315 | 129.715 | 1.00 | 29.07 | A | C |
| ATOM | 1684 | CD2 | TYR | 309 | −3.061 | −32.443 | 132.162 | 1.00 | 29.31 | A | C |
| ATOM | 1685 | CE2 | TYR | 309 | −2.209 | −31.511 | 131.573 | 1.00 | 29.09 | A | C |
| ATOM | 1686 | CZ | TYR | 309 | −2.544 | −30.955 | 130.353 | 1.00 | 28.97 | A | C |
| ATOM | 1687 | OH | TYR | 309 | −1.697 | −30.049 | 129.766 | 1.00 | 29.80 | A | O |
| ATOM | 1688 | C | TYR | 309 | −3.842 | −35.738 | 131.198 | 1.00 | 27.07 | A | C |
| ATOM | 1689 | O | TYR | 309 | −3.204 | −35.558 | 130.169 | 1.00 | 26.88 | A | O |
| ATOM | 1690 | N | ALA | 310 | −3.368 | −36.436 | 132.225 | 1.00 | 25.89 | A | N |
| ATOM | 1691 | CA | ALA | 310 | −2.049 | −37.051 | 132.169 | 1.00 | 25.16 | A | C |
| ATOM | 1692 | CB | ALA | 310 | −1.702 | −37.690 | 133.512 | 1.00 | 24.32 | A | C |
| ATOM | 1693 | C | ALA | 310 | −1.997 | −38.096 | 131.046 | 1.00 | 24.96 | A | C |
| ATOM | 1694 | O | ALA | 310 | −1.023 | −38.142 | 130.291 | 1.00 | 24.12 | A | O |
| ATOM | 1695 | N | LYS | 311 | −3.036 | −38.930 | 130.940 | 1.00 | 23.97 | A | N |
| ATOM | 1696 | CA | LYS | 311 | −3.097 | −39.938 | 129.881 | 1.00 | 24.13 | A | C |
| ATOM | 1697 | CB | LYS | 311 | −4.391 | −40.752 | 129.960 | 1.00 | 25.00 | A | C |
| ATOM | 1698 | CG | LYS | 311 | −4.473 | −41.760 | 131.105 | 1.00 | 27.78 | A | C |
| ATOM | 1699 | CD | LYS | 311 | −5.831 | −42.472 | 131.058 | 1.00 | 29.79 | A | C |
| ATOM | 1700 | CE | LYS | 311 | −6.089 | −43.328 | 132.285 | 1.00 | 31.20 | A | C |
| ATOM | 1701 | NZ | LYS | 311 | −7.505 | −43.836 | 132.302 | 1.00 | 33.50 | A | N |
| ATOM | 1702 | C | LYS | 311 | −3.031 | −39.268 | 128.508 | 1.00 | 23.70 | A | C |
| ATOM | 1703 | O | LYS | 311 | −2.430 | −39.806 | 127.583 | 1.00 | 23.05 | A | O |
| ATOM | 1704 | N | LEU | 312 | −3.651 | −38.098 | 128.378 | 1.00 | 22.94 | A | N |
| ATOM | 1705 | CA | LEU | 312 | −3.643 | −37.380 | 127.113 | 1.00 | 23.55 | A | C |
| ATOM | 1706 | CB | LEU | 312 | −4.656 | −36.230 | 127.143 | 1.00 | 24.36 | A | C |
| ATOM | 1707 | CG | LEU | 312 | −6.128 | −36.661 | 127.073 | 1.00 | 25.69 | A | C |
| ATOM | 1708 | CD1 | LEU | 312 | −7.026 | −35.447 | 126.879 | 1.00 | 24.81 | A | C |
| ATOM | 1709 | CD2 | LEU | 312 | −6.312 | −37.629 | 125.910 | 1.00 | 26.15 | A | C |
| ATOM | 1710 | C | LEU | 312 | −2.248 | −36.860 | 126.748 | 1.00 | 23.28 | A | C |
| ATOM | 1711 | O | LEU | 312 | −1.859 | −36.888 | 125.580 | 1.00 | 22.28 | A | O |
| ATOM | 1712 | N | LEU | 313 | −1.498 | −36.374 | 127.733 | 1.00 | 22.16 | A | N |
| ATOM | 1713 | CA | LEU | 313 | −0.145 | −35.905 | 127.460 | 1.00 | 22.07 | A | C |
| ATOM | 1714 | CB | LEU | 313 | 0.454 | −35.225 | 128.694 | 1.00 | 21.00 | A | C |
| ATOM | 1715 | CG | LEU | 313 | −0.202 | −33.891 | 129.064 | 1.00 | 21.22 | A | C |
| ATOM | 1716 | CD1 | LEU | 313 | 0.485 | −33.316 | 130.284 | 1.00 | 20.26 | A | C |
| ATOM | 1717 | CD2 | LEU | 313 | −0.115 | −32.920 | 127.886 | 1.00 | 18.10 | A | C |
| ATOM | 1718 | C | LEU | 313 | 0.681 | −37.131 | 127.059 | 1.00 | 22.48 | A | C |
| ATOM | 1719 | O | LEU | 313 | 1.512 | −37.068 | 126.152 | 1.00 | 22.20 | A | O |
| ATOM | 1720 | N | GLY | 314 | 0.434 | −38.251 | 127.733 | 1.00 | 22.89 | A | N |
| ATOM | 1721 | CA | GLY | 314 | 1.131 | −39.479 | 127.402 | 1.00 | 23.30 | A | C |
| ATOM | 1722 | C | GLY | 314 | 0.860 | −39.842 | 125.949 | 1.00 | 23.94 | A | C |
| ATOM | 1723 | O | GLY | 314 | 1.782 | −40.168 | 125.200 | 1.00 | 24.54 | A | O |
| ATOM | 1724 | N | LEU | 315 | −0.410 | −39.779 | 125.549 | 1.00 | 23.59 | A | N |
| ATOM | 1725 | CA | LEU | 315 | −0.807 | −40.092 | 124.182 | 1.00 | 22.89 | A | C |
| ATOM | 1726 | CB | LEU | 315 | −2.336 | −40.134 | 124.070 | 1.00 | 23.44 | A | C |
| ATOM | 1727 | CG | LEU | 315 | −2.961 | −41.398 | 124.681 | 1.00 | 23.97 | A | C |
| ATOM | 1728 | CD1 | LEU | 315 | −4.465 | −41.270 | 124.742 | 1.00 | 23.92 | A | C |
| ATOM | 1729 | CD2 | LEU | 315 | −2.566 | −42.613 | 123.854 | 1.00 | 23.72 | A | C |
| ATOM | 1730 | C | LEU | 315 | −0.221 | −39.115 | 123.167 | 1.00 | 22.47 | A | C |
| ATOM | 1731 | O | LEU | 315 | 0.094 | −39.505 | 122.044 | 1.00 | 22.11 | A | O |
| ATOM | 1732 | N | LEU | 316 | −0.072 | −37.849 | 123.544 | 1.00 | 22.35 | A | N |
| ATOM | 1733 | CA | LEU | 316 | 0.524 | −36.882 | 122.626 | 1.00 | 22.93 | A | C |
| ATOM | 1734 | CB | LEU | 316 | 0.454 | −35.465 | 123.194 | 1.00 | 23.82 | A | C |
| ATOM | 1735 | CG | LEU | 316 | −0.934 | −34.817 | 123.164 | 1.00 | 25.69 | A | C |
| ATOM | 1736 | CD1 | LEU | 316 | −0.879 | −33.440 | 123.802 | 1.00 | 25.97 | A | C |
| ATOM | 1737 | CD2 | LEU | 316 | −1.418 | −34.720 | 121.722 | 1.00 | 26.87 | A | C |
| ATOM | 1738 | C | LEU | 316 | 1.977 | −37.273 | 122.401 | 1.00 | 23.05 | A | C |
| ATOM | 1739 | O | LEU | 316 | 2.496 | −37.162 | 121.290 | 1.00 | 23.13 | A | O |
| ATOM | 1740 | N | ALA | 317 | 2.624 | −37.741 | 123.464 | 1.00 | 22.68 | A | N |
| ATOM | 1741 | CA | ALA | 317 | 4.009 | −38.169 | 123.392 | 1.00 | 23.73 | A | C |
| ATOM | 1742 | CB | ALA | 317 | 4.560 | −38.439 | 124.804 | 1.00 | 23.13 | A | C |
| ATOM | 1743 | C | ALA | 317 | 4.111 | −39.426 | 122.536 | 1.00 | 23.75 | A | C |
| ATOM | 1744 | O | ALA | 317 | 5.043 | −39.567 | 121.759 | 1.00 | 22.15 | A | O |
| ATOM | 1745 | N | GLU | 318 | 3.150 | −40.334 | 122.667 | 1.00 | 24.36 | A | N |
| ATOM | 1746 | CA | GLU | 318 | 3.183 | −41.562 | 121.881 | 1.00 | 26.08 | A | C |
| ATOM | 1747 | CB | GLU | 318 | 2.115 | −42.543 | 122.362 | 1.00 | 28.21 | A | C |
| ATOM | 1748 | CG | GLU | 318 | 2.116 | −43.831 | 121.566 | 1.00 | 32.51 | A | C |
| ATOM | 1749 | CD | GLU | 318 | 1.632 | −45.004 | 122.375 | 1.00 | 35.59 | A | C |
| ATOM | 1750 | OE1 | GLU | 318 | 0.410 | −45.114 | 122.604 | 1.00 | 36.82 | A | O |
| ATOM | 1751 | OE2 | GLU | 318 | 2.488 | −45.811 | 122.796 | 1.00 | 38.53 | A | O |
| ATOM | 1752 | C | GLU | 318 | 2.997 | −41.300 | 120.391 | 1.00 | 25.72 | A | C |
| ATOM | 1753 | O | GLU | 318 | 3.641 | −41.929 | 119.557 | 1.00 | 24.88 | A | O |
| ATOM | 1754 | N | LEU | 319 | 2.101 | −40.375 | 120.068 | 1.00 | 25.55 | A | N |
| ATOM | 1755 | CA | LEU | 319 | 1.832 | −40.001 | 118.684 | 1.00 | 25.73 | A | C |
| ATOM | 1756 | CB | LEU | 319 | 0.684 | −38.994 | 118.652 | 1.00 | 24.34 | A | C |
| ATOM | 1757 | CG | LEU | 319 | 0.123 | −38.538 | 117.311 | 1.00 | 24.57 | A | C |
| ATOM | 1758 | CD1 | LEU | 319 | −0.173 | −39.730 | 116.426 | 1.00 | 21.78 | A | C |
| ATOM | 1759 | CD2 | LEU | 319 | −1.131 | −37.713 | 117.576 | 1.00 | 23.45 | A | C |
| ATOM | 1760 | C | LEU | 319 | 3.112 | −39.395 | 118.103 | 1.00 | 26.16 | A | C |
| ATOM | 1761 | O | LEU | 319 | 3.389 | −39.500 | 116.907 | 1.00 | 26.27 | A | O |
| ATOM | 1762 | N | ARG | 320 | 3.889 | −38.757 | 118.969 | 1.00 | 26.63 | A | N |
| ATOM | 1763 | CA | ARG | 320 | 5.159 | −38.169 | 118.587 | 1.00 | 27.47 | A | C |
| ATOM | 1764 | CB | ARG | 320 | 5.750 | −37.430 | 119.786 | 1.00 | 29.62 | A | C |
| ATOM | 1765 | CG | ARG | 320 | 6.936 | −36.574 | 119.463 | 1.00 | 32.70 | A | C |
| ATOM | 1766 | CD | ARG | 320 | 6.505 | −35.319 | 118.741 | 1.00 | 35.19 | A | C |
| ATOM | 1767 | NE | ARG | 320 | 7.665 | −34.554 | 118.307 | 1.00 | 36.76 | A | N |
| ATOM | 1768 | CZ | ARG | 320 | 7.899 | −33.287 | 118.624 | 1.00 | 37.53 | A | C |
| ATOM | 1769 | NH1 | ARG | 320 | 7.054 | −32.609 | 119.389 | 1.00 | 37.83 | A | N |
| ATOM | 1770 | NH2 | ARG | 320 | 8.993 | −32.697 | 118.166 | 1.00 | 39.80 | A | N |
| ATOM | 1771 | C | ARG | 320 | 6.081 | −39.326 | 118.169 | 1.00 | 26.95 | A | C |
| ATOM | 1772 | O | ARG | 320 | 6.849 | −39.209 | 117.209 | 1.00 | 27.09 | A | O |
| ATOM | 1773 | N | SER | 321 | 5.996 | −40.441 | 118.898 | 1.00 | 25.51 | A | N |
| ATOM | 1774 | CA | SER | 321 | 6.790 | −41.638 | 118.603 | 1.00 | 25.26 | A | C |
| ATOM | 1775 | CB | SER | 321 | 6.577 | −42.720 | 119.661 | 1.00 | 24.40 | A | C |
| ATOM | 1776 | OG | SER | 321 | 7.275 | −42.419 | 120.843 | 1.00 | 26.98 | A | O |
| ATOM | 1777 | C | SER | 321 | 6.377 | −42.210 | 117.261 | 1.00 | 24.22 | A | C |
| ATOM | 1778 | O | SER | 321 | 7.219 | −42.588 | 116.450 | 1.00 | 23.84 | A | O |
| ATOM | 1779 | N | ILE | 322 | 5.069 | −42.293 | 117.051 | 1.00 | 23.23 | A | N |
| ATOM | 1780 | CA | ILE | 322 | 4.521 | −42.802 | 115.810 | 1.00 | 23.56 | A | C |
| ATOM | 1781 | CB | ILE | 322 | 2.979 | −42.784 | 115.876 | 1.00 | 23.28 | A | C |
| ATOM | 1782 | CG2 | ILE | 322 | 2.377 | −42.970 | 114.484 | 1.00 | 22.26 | A | C |
| ATOM | 1783 | CG1 | ILE | 322 | 2.513 | −43.873 | 116.855 | 1.00 | 22.75 | A | C |
| ATOM | 1784 | CD1 | ILE | 322 | 1.021 | −43.879 | 117.140 | 1.00 | 21.11 | A | C |
| ATOM | 1785 | C | ILE | 322 | 5.024 | −41.955 | 114.639 | 1.00 | 24.10 | A | C |
| ATOM | 1786 | O | ILE | 322 | 5.387 | −42.489 | 113.590 | 1.00 | 23.74 | A | O |
| ATOM | 1787 | N | ASN | 323 | 5.055 | −40.637 | 114.826 | 1.00 | 24.50 | A | N |
| ATOM | 1788 | CA | ASN | 323 | 5.523 | −39.724 | 113.787 | 1.00 | 25.76 | A | C |
| ATOM | 1789 | CB | ASN | 323 | 5.477 | −38.280 | 114.295 | 1.00 | 27.72 | A | C |
| ATOM | 1790 | CG | ASN | 323 | 5.909 | −37.271 | 113.244 | 1.00 | 30.34 | A | C |
| ATOM | 1791 | OD1 | ASN | 323 | 6.870 | −37.489 | 112.509 | 1.00 | 33.18 | A | O |
| ATOM | 1792 | ND2 | ASN | 323 | 5.215 | −36.148 | 113.189 | 1.00 | 31.75 | A | N |
| ATOM | 1793 | C | ASN | 323 | 6.953 | −40.100 | 113.408 | 1.00 | 25.95 | A | C |
| ATOM | 1794 | O | ASN | 323 | 7.265 | −40.282 | 112.236 | 1.00 | 25.34 | A | O |
| ATOM | 1795 | N | GLU | 324 | 7.817 | −40.232 | 114.410 | 1.00 | 26.65 | A | N |
| ATOM | 1796 | CA | GLU | 324 | 9.208 | −40.597 | 114.173 | 1.00 | 27.58 | A | C |
| ATOM | 1797 | CB | GLU | 324 | 9.994 | −40.535 | 115.482 | 1.00 | 30.21 | A | C |
| ATOM | 1798 | CG | GLU | 324 | 9.920 | −39.180 | 116.165 | 1.00 | 34.60 | A | C |
| ATOM | 1799 | CD | GLU | 324 | 10.646 | −39.163 | 117.490 | 1.00 | 37.84 | A | C |
| ATOM | 1800 | OE1 | GLU | 324 | 10.494 | −40.138 | 118.258 | 1.00 | 39.66 | A | O |
| ATOM | 1801 | OE2 | GLU | 324 | 11.359 | −38.173 | 117.770 | 1.00 | 39.51 | A | O |
| ATOM | 1802 | C | GLU | 324 | 9.343 | −41.988 | 113.549 | 1.00 | 26.74 | A | C |
| ATOM | 1803 | O | GLU | 324 | 10.238 | −42.221 | 112.740 | 1.00 | 26.19 | A | O |
| ATOM | 1804 | N | ALA | 325 | 8.464 | −42.914 | 113.923 | 1.00 | 25.09 | A | N |
| ATOM | 1805 | CA | ALA | 325 | 8.517 | −44.258 | 113.359 | 1.00 | 25.00 | A | C |
| ATOM | 1806 | CB | ALA | 325 | 7.619 | −45.209 | 114.153 | 1.00 | 24.73 | A | C |
| ATOM | 1807 | C | ALA | 325 | 8.111 | −44.238 | 111.878 | 1.00 | 24.89 | A | C |
| ATOM | 1808 | O | ALA | 325 | 8.561 | −45.073 | 111.093 | 1.00 | 25.47 | A | O |
| ATOM | 1809 | N | TYR | 326 | 7.250 | −43.297 | 111.502 | 1.00 | 23.96 | A | N |
| ATOM | 1810 | CA | TYR | 326 | 6.845 | −43.145 | 110.106 | 1.00 | 23.70 | A | C |
| ATOM | 1811 | CB | TYR | 326 | 5.856 | −41.988 | 109.952 | 1.00 | 22.03 | A | C |
| ATOM | 1812 | CG | TYR | 326 | 4.413 | −42.402 | 109.901 | 1.00 | 21.43 | A | C |
| ATOM | 1813 | CD1 | TYR | 326 | 3.922 | −43.180 | 108.848 | 1.00 | 20.69 | A | C |
| ATOM | 1814 | CE1 | TYR | 326 | 2.571 | −43.541 | 108.787 | 1.00 | 19.55 | A | C |
| ATOM | 1815 | CD2 | TYR | 326 | 3.521 | −41.996 | 110.897 | 1.00 | 21.09 | A | C |
| ATOM | 1816 | CE2 | TYR | 326 | 2.175 | −42.348 | 110.845 | 1.00 | 20.21 | A | C |
| ATOM | 1817 | CZ | TYR | 326 | 1.707 | −43.117 | 109.791 | 1.00 | 20.21 | A | C |
| ATOM | 1818 | OH | TYR | 326 | 0.374 | −43.439 | 109.749 | 1.00 | 19.33 | A | O |
| ATOM | 1819 | C | TYR | 326 | 8.093 | −42.827 | 109.280 | 1.00 | 24.16 | A | C |
| ATOM | 1820 | O | TYR | 326 | 8.276 | −43.360 | 108.196 | 1.00 | 23.33 | A | O |
| ATOM | 1821 | N | GLY | 327 | 8.933 | −41.936 | 109.802 | 1.00 | 25.27 | A | N |
| ATOM | 1822 | CA | GLY | 327 | 10.158 | −41.563 | 109.116 | 1.00 | 26.47 | A | C |
| ATOM | 1823 | C | GLY | 327 | 11.054 | −42.765 | 108.912 | 1.00 | 27.58 | A | C |
| ATOM | 1824 | O | GLY | 327 | 11.633 | −42.937 | 107.848 | 1.00 | 27.44 | A | O |
| ATOM | 1825 | N | TYR | 328 | 11.176 | −43.601 | 109.938 | 1.00 | 29.10 | A | N |
| ATOM | 1826 | CA | TYR | 328 | 11.992 | −44.801 | 109.838 | 1.00 | 30.30 | A | C |
| ATOM | 1827 | CB | TYR | 328 | 12.018 | −45.537 | 111.182 | 1.00 | 31.95 | A | C |
| ATOM | 1828 | CG | TYR | 328 | 12.753 | −46.859 | 111.127 | 1.00 | 33.79 | A | C |
| ATOM | 1829 | CD1 | TYR | 328 | 14.129 | −46.930 | 111.361 | 1.00 | 35.24 | A | C |
| ATOM | 1830 | CE1 | TYR | 328 | 14.819 | −48.144 | 111.242 | 1.00 | 36.06 | A | C |
| ATOM | 1831 | CD2 | TYR | 328 | 12.084 | −48.032 | 110.778 | 1.00 | 34.19 | A | C |
| ATOM | 1832 | CE2 | TYR | 328 | 12.760 | −49.243 | 110.652 | 1.00 | 35.21 | A | C |
| ATOM | 1833 | CZ | TYR | 328 | 14.126 | −49.293 | 110.884 | 1.00 | 36.45 | A | C |
| ATOM | 1834 | OH | TYR | 328 | 14.797 | −50.489 | 110.736 | 1.00 | 37.83 | A | O |
| ATOM | 1835 | C | TYR | 328 | 11.411 | −45.722 | 108.758 | 1.00 | 30.82 | A | C |
| ATOM | 1836 | O | TYR | 328 | 12.145 | −46.306 | 107.966 | 1.00 | 30.88 | A | O |
| ATOM | 1837 | N | GLN | 329 | 10.088 | −45.852 | 108.737 | 1.00 | 31.03 | A | N |
| ATOM | 1838 | CA | GLN | 329 | 9.412 | −46.699 | 107.759 | 1.00 | 31.18 | A | C |
| ATOM | 1839 | CB | GLN | 329 | 7.902 | −46.700 | 108.011 | 1.00 | 29.87 | A | C |
| ATOM | 1840 | CG | GLN | 329 | 7.486 | −47.228 | 109.377 | 1.00 | 30.38 | A | C |
| ATOM | 1841 | CD | GLN | 329 | 7.716 | −48.717 | 109.523 | 1.00 | 31.26 | A | C |
| ATOM | 1842 | OE1 | GLN | 329 | 8.533 | −49.168 | 110.338 | 1.00 | 30.58 | A | O |
| ATOM | 1843 | NE2 | GLN | 329 | 6.995 | −49.496 | 108.729 | 1.00 | 30.24 | A | N |
| ATOM | 1844 | C | GLN | 329 | 9.688 | −46.220 | 106.338 | 1.00 | 31.84 | A | C |
| ATOM | 1845 | O | GLN | 329 | 10.002 | −47.017 | 105.457 | 1.00 | 31.67 | A | O |
| ATOM | 1846 | N | ILE | 330 | 9.565 | −44.916 | 106.118 | 1.00 | 32.78 | A | N |
| ATOM | 1847 | CA | ILE | 330 | 9.796 | −44.336 | 104.800 | 1.00 | 34.51 | A | C |
| ATOM | 1848 | CB | ILE | 330 | 9.423 | −42.833 | 104.798 | 1.00 | 34.57 | A | C |
| ATOM | 1849 | CG2 | ILE | 330 | 10.061 | −42.113 | 103.613 | 1.00 | 34.87 | A | C |
| ATOM | 1850 | CG1 | ILE | 330 | 7.900 | −42.697 | 104.752 | 1.00 | 34.64 | A | C |
| ATOM | 1851 | CD1 | ILE | 330 | 7.407 | −41.267 | 104.779 | 1.00 | 35.62 | A | C |
| ATOM | 1852 | C | ILE | 330 | 11.231 | −44.519 | 104.300 | 1.00 | 35.74 | A | C |
| ATOM | 1853 | O | ILE | 330 | 11.467 | −44.599 | 103.094 | 1.00 | 34.91 | A | O |
| ATOM | 1854 | N | GLN | 331 | 12.179 | −44.604 | 105.228 | 1.00 | 37.46 | A | N |
| ATOM | 1855 | CA | GLN | 331 | 13.586 | −44.776 | 104.882 | 1.00 | 39.69 | A | C |
| ATOM | 1856 | CB | GLN | 331 | 14.473 | −44.235 | 105.999 | 1.00 | 42.13 | A | C |
| ATOM | 1857 | CG | GLN | 331 | 14.507 | −42.735 | 106.134 | 1.00 | 44.67 | A | C |
| ATOM | 1858 | CD | GLN | 331 | 15.164 | −42.325 | 107.428 | 1.00 | 47.11 | A | C |
| ATOM | 1859 | OE1 | GLN | 331 | 16.202 | −42.870 | 107.806 | 1.00 | 48.26 | A | O |
| ATOM | 1860 | NE2 | GLN | 331 | 14.565 | −41.362 | 108.120 | 1.00 | 48.66 | A | N |
| ATOM | 1861 | C | GLN | 331 | 14.010 | −46.211 | 104.613 | 1.00 | 39.80 | A | C |
| ATOM | 1862 | O | GLN | 331 | 14.843 | −46.463 | 103.749 | 1.00 | 40.44 | A | O |
| ATOM | 1863 | N | HIS | 332 | 13.446 | −47.155 | 105.351 | 1.00 | 39.86 | A | N |
| ATOM | 1864 | CA | HIS | 332 | 13.839 | −48.546 | 105.187 | 1.00 | 40.67 | A | C |
| ATOM | 1865 | CB | HIS | 332 | 14.048 | −49.161 | 106.571 | 1.00 | 42.81 | A | C |
| ATOM | 1866 | CG | HIS | 332 | 15.172 | −48.524 | 107.331 | 1.00 | 46.28 | A | C |
| ATOM | 1867 | CD2 | HIS | 332 | 15.209 | −47.399 | 108.086 | 1.00 | 47.01 | A | C |
| ATOM | 1868 | ND1 | HIS | 332 | 16.466 | −48.992 | 107.275 | 1.00 | 47.57 | A | N |
| ATOM | 1869 | CE1 | HIS | 332 | 17.256 | −48.181 | 107.961 | 1.00 | 47.73 | A | C |
| ATOM | 1870 | NE2 | HIS | 332 | 16.518 | −47.208 | 108.461 | 1.00 | 48.10 | A | N |
| ATOM | 1871 | C | HIS | 332 | 12.928 | −49.422 | 104.337 | 1.00 | 39.76 | A | C |
| ATOM | 1872 | O | HIS | 332 | 13.241 | −50.587 | 104.082 | 1.00 | 39.61 | A | O |
| ATOM | 1873 | N | ILE | 333 | 11.810 | −48.871 | 103.882 | 1.00 | 38.23 | A | N |
| ATOM | 1874 | CA | ILE | 333 | 10.900 | −49.647 | 103.055 | 1.00 | 37.05 | A | C |
| ATOM | 1875 | CB | ILE | 333 | 9.567 | −49.887 | 103.779 | 1.00 | 36.60 | A | C |
| ATOM | 1876 | CG2 | ILE | 333 | 8.618 | −50.652 | 102.878 | 1.00 | 36.04 | A | C |
| ATOM | 1877 | CG1 | ILE | 333 | 9.824 | −50.680 | 105.066 | 1.00 | 37.04 | A | C |
| ATOM | 1878 | CD1 | ILE | 333 | 8.620 | −50.826 | 105.963 | 1.00 | 36.04 | A | C |
| ATOM | 1879 | C | ILE | 333 | 10.656 | −48.967 | 101.713 | 1.00 | 36.44 | A | C |
| ATOM | 1880 | O | ILE | 333 | 9.841 | −48.056 | 101.599 | 1.00 | 36.09 | A | O |
| ATOM | 1881 | N | GLN | 334 | 11.394 | −49.424 | 100.707 | 1.00 | 35.60 | A | N |
| ATOM | 1882 | CA | GLN | 334 | 11.309 | −48.909 | 99.345 | 1.00 | 35.11 | A | C |
| ATOM | 1883 | CB | GLN | 334 | 12.172 | −49.788 | 98.427 | 1.00 | 37.05 | A | C |
| ATOM | 1884 | CG | GLN | 334 | 12.764 | −49.113 | 97.192 | 1.00 | 40.94 | A | C |
| ATOM | 1885 | CD | GLN | 334 | 11.771 | −48.947 | 96.055 | 1.00 | 43.49 | A | C |
| ATOM | 1886 | OE1 | GLN | 334 | 10.952 | −48.031 | 96.060 | 1.00 | 45.60 | A | O |
| ATOM | 1887 | NE2 | GLN | 334 | 11.838 | −49.846 | 95.071 | 1.00 | 44.60 | A | N |
| ATOM | 1888 | C | GLN | 334 | 9.851 | −48.925 | 98.888 | 1.00 | 33.48 | A | C |
| ATOM | 1889 | O | GLN | 334 | 9.151 | −49.929 | 99.036 | 1.00 | 32.53 | A | O |
| ATOM | 1890 | N | GLY | 335 | 9.388 | −47.801 | 98.353 | 1.00 | 32.61 | A | N |
| ATOM | 1891 | CA | GLY | 335 | 8.019 | −47.724 | 97.879 | 1.00 | 31.27 | A | C |
| ATOM | 1892 | C | GLY | 335 | 7.029 | −47.059 | 98.816 | 1.00 | 30.52 | A | C |
| ATOM | 1893 | O | GLY | 335 | 6.029 | −46.519 | 98.357 | 1.00 | 29.45 | A | O |
| ATOM | 1894 | N | LEU | 336 | 7.289 | −47.091 | 100.122 | 1.00 | 30.35 | A | N |
| ATOM | 1895 | CA | LEU | 336 | 6.371 | −46.474 | 101.073 | 1.00 | 30.47 | A | C |
| ATOM | 1896 | CB | LEU | 336 | 6.837 | −46.683 | 102.515 | 1.00 | 30.60 | A | C |
| ATOM | 1897 | CG | LEU | 336 | 6.505 | −47.973 | 103.265 | 1.00 | 31.59 | A | C |
| ATOM | 1898 | CD1 | LEU | 336 | 6.881 | −47.777 | 104.731 | 1.00 | 32.05 | A | C |
| ATOM | 1899 | CD2 | LEU | 336 | 5.027 | −48.308 | 103.153 | 1.00 | 31.01 | A | C |
| ATOM | 1900 | C | LEU | 336 | 6.186 | −44.983 | 100.845 | 1.00 | 30.50 | A | C |
| ATOM | 1901 | O | LEU | 336 | 5.066 | −44.487 | 100.891 | 1.00 | 29.66 | A | O |
| ATOM | 1902 | N | SER | 337 | 7.283 | −44.271 | 100.603 | 1.00 | 31.26 | A | N |
| ATOM | 1903 | CA | SER | 337 | 7.224 | −42.829 | 100.395 | 1.00 | 32.62 | A | C |
| ATOM | 1904 | CB | SER | 337 | 8.628 | −42.267 | 100.141 | 1.00 | 33.56 | A | C |
| ATOM | 1905 | OG | SER | 337 | 9.184 | −42.777 | 98.943 | 1.00 | 35.00 | A | O |
| ATOM | 1906 | C | SER | 337 | 6.286 | −42.419 | 99.263 | 1.00 | 32.68 | A | C |
| ATOM | 1907 | O | SER | 337 | 5.792 | −41.300 | 99.246 | 1.00 | 33.22 | A | O |
| ATOM | 1908 | N | ALA | 338 | 6.030 | −43.321 | 98.324 | 1.00 | 33.04 | A | N |
| ATOM | 1909 | CA | ALA | 338 | 5.133 | −43.016 | 97.218 | 1.00 | 33.69 | A | C |
| ATOM | 1910 | CB | ALA | 338 | 5.072 | −44.199 | 96.257 | 1.00 | 33.20 | A | C |
| ATOM | 1911 | C | ALA | 338 | 3.723 | −42.669 | 97.711 | 1.00 | 34.44 | A | C |
| ATOM | 1912 | O | ALA | 338 | 2.979 | −41.952 | 97.032 | 1.00 | 34.62 | A | O |
| ATOM | 1913 | N | MET | 339 | 3.348 | −43.186 | 98.879 | 1.00 | 34.28 | A | N |
| ATOM | 1914 | CA | MET | 339 | 2.024 | −42.906 | 99.422 | 1.00 | 35.53 | A | C |
| ATOM | 1915 | CB | MET | 339 | 1.604 | −44.003 | 100.400 | 1.00 | 31.99 | A | C |
| ATOM | 1916 | CG | MET | 339 | 1.203 | −45.292 | 99.701 | 1.00 | 29.56 | A | C |
| ATOM | 1917 | SD | MET | 339 | 0.803 | −46.628 | 100.838 | 1.00 | 26.76 | A | S |
| ATOM | 1918 | CE | MET | 339 | 2.486 | −47.154 | 101.313 | 1.00 | 25.89 | A | C |
| ATOM | 1919 | C | MET | 339 | 1.956 | −41.537 | 100.086 | 1.00 | 38.11 | A | C |
| ATOM | 1920 | O | MET | 339 | 0.883 | −41.074 | 100.475 | 1.00 | 38.51 | A | O |
| ATOM | 1921 | N | MET | 340 | 3.108 | −40.891 | 100.217 | 1.00 | 41.19 | A | N |
| ATOM | 1922 | CA | MET | 340 | 3.162 | −39.564 | 100.792 | 1.00 | 45.17 | A | C |
| ATOM | 1923 | CB | MET | 340 | 3.933 | −39.576 | 102.108 | 1.00 | 45.06 | A | C |
| ATOM | 1924 | CG | MET | 340 | 3.761 | −38.294 | 102.892 | 1.00 | 45.91 | A | C |
| ATOM | 1925 | SD | MET | 340 | 2.006 | −37.937 | 103.173 | 1.00 | 46.05 | A | S |
| ATOM | 1926 | CE | MET | 340 | 1.556 | −37.032 | 101.643 | 1.00 | 45.26 | A | C |
| ATOM | 1927 | C | MET | 340 | 3.848 | −38.636 | 99.793 | 1.00 | 48.30 | A | C |
| ATOM | 1928 | O | MET | 340 | 4.965 | −38.182 | 100.023 | 1.00 | 48.09 | A | O |
| ATOM | 1929 | N | PRO | 341 | 3.180 | −38.348 | 98.662 | 1.00 | 51.89 | A | N |
| ATOM | 1930 | CD | PRO | 341 | 1.818 | −38.785 | 98.307 | 1.00 | 52.61 | A | C |
| ATOM | 1931 | CA | PRO | 341 | 3.725 | −37.474 | 97.616 | 1.00 | 55.17 | A | C |
| ATOM | 1932 | CB | PRO | 341 | 2.552 | −37.321 | 96.649 | 1.00 | 54.46 | A | C |
| ATOM | 1933 | CG | PRO | 341 | 1.812 | −38.613 | 96.810 | 1.00 | 53.80 | A | C |
| ATOM | 1934 | C | PRO | 341 | 4.201 | −36.129 | 98.151 | 1.00 | 58.43 | A | C |
| ATOM | 1935 | O | PRO | 341 | 5.399 | −35.838 | 98.177 | 1.00 | 58.94 | A | O |
| ATOM | 1936 | N | LEU | 342 | 3.244 | −35.312 | 98.573 | 1.00 | 61.89 | A | N |
| ATOM | 1937 | CA | LEU | 342 | 3.538 | −33.993 | 99.108 | 1.00 | 65.33 | A | C |
| ATOM | 1938 | CB | LEU | 342 | 2.297 | −33.094 | 99.015 | 1.00 | 65.46 | A | C |
| ATOM | 1939 | CG | LEU | 342 | 1.863 | −32.488 | 97.675 | 1.00 | 65.98 | A | C |
| ATOM | 1940 | CD1 | LEU | 342 | 1.636 | −33.558 | 96.613 | 1.00 | 66.50 | A | C |
| ATOM | 1941 | CD2 | LEU | 342 | 0.586 | −31.702 | 97.907 | 1.00 | 66.63 | A | C |
| ATOM | 1942 | C | LEU | 342 | 3.956 | −34.121 | 100.563 | 1.00 | 67.54 | A | C |
| ATOM | 1943 | O | LEU | 342 | 3.808 | −35.183 | 101.167 | 1.00 | 67.71 | A | O |
| ATOM | 1944 | N | LEU | 343 | 4.490 | −33.033 | 101.110 | 1.00 | 70.31 | A | N |
| ATOM | 1945 | CA | LEU | 343 | 4.909 | −32.986 | 102.505 | 1.00 | 73.10 | A | C |
| ATOM | 1946 | CB | LEU | 343 | 3.669 | −32.854 | 103.401 | 1.00 | 73.06 | A | C |
| ATOM | 1947 | CG | LEU | 343 | 3.783 | −32.206 | 104.783 | 1.00 | 73.13 | A | C |
| ATOM | 1948 | CD1 | LEU | 343 | 4.851 | −32.903 | 105.615 | 1.00 | 73.35 | A | C |
| ATOM | 1949 | CD2 | LEU | 343 | 4.111 | −30.733 | 104.613 | 1.00 | 73.19 | A | C |
| ATOM | 1950 | C | LEU | 343 | 5.712 | −34.220 | 102.919 | 1.00 | 75.17 | A | C |
| ATOM | 1951 | O | LEU | 343 | 5.138 | −35.263 | 103.242 | 1.00 | 75.75 | A | O |
| ATOM | 1952 | N | GLN | 344 | 7.037 | −34.095 | 102.908 | 1.00 | 77.19 | A | N |
| ATOM | 1953 | CA | GLN | 344 | 7.928 | −35.185 | 103.303 | 1.00 | 79.02 | A | C |
| ATOM | 1954 | CB | GLN | 344 | 7.588 | −36.483 | 102.552 | 1.00 | 79.26 | A | C |
| ATOM | 1955 | CG | GLN | 344 | 7.934 | −36.476 | 101.062 | 1.00 | 79.55 | A | C |
| ATOM | 1956 | CD | GLN | 344 | 8.169 | −37.876 | 100.495 | 1.00 | 79.92 | A | C |
| ATOM | 1957 | OE1 | GLN | 344 | 8.352 | −38.044 | 99.288 | 1.00 | 80.00 | A | O |
| ATOM | 1958 | NE2 | GLN | 344 | 8.173 | −38.884 | 101.366 | 1.00 | 79.76 | A | N |
| ATOM | 1959 | C | GLN | 344 | 9.386 | −34.823 | 103.034 | 1.00 | 80.26 | A | C |
| ATOM | 1960 | O | GLN | 344 | 9.691 | −33.714 | 102.587 | 1.00 | 80.47 | A | O |
| ATOM | 1961 | N | GLU | 345 | 10.283 | −35.766 | 103.314 | 1.00 | 81.48 | A | N |
| ATOM | 1962 | CA | GLU | 345 | 11.710 | −35.565 | 103.093 | 1.00 | 82.57 | A | C |
| ATOM | 1963 | CB | GLU | 345 | 12.522 | −36.275 | 104.182 | 1.00 | 83.18 | A | C |
| ATOM | 1964 | CG | GLU | 345 | 12.229 | −35.802 | 105.598 | 1.00 | 84.08 | A | C |
| ATOM | 1965 | CD | GLU | 345 | 13.093 | −36.500 | 106.634 | 1.00 | 84.62 | A | C |
| ATOM | 1966 | OE1 | GLU | 345 | 13.032 | −37.746 | 106.722 | 1.00 | 85.08 | A | O |
| ATOM | 1967 | OE2 | GLU | 345 | 13.834 | −35.801 | 107.359 | 1.00 | 84.92 | A | O |
| ATOM | 1968 | C | GLU | 345 | 12.106 | −36.112 | 101.721 | 1.00 | 82.85 | A | C |
| ATOM | 1969 | O | GLU | 345 | 13.062 | −36.916 | 101.657 | 1.00 | 83.15 | A | O |
| ATOM | 1970 | OXT | GLU | 345 | 11.457 | −35.728 | 100.723 | 1.00 | 83.01 | A | O |
| TER | 1971 | | GLU | 345 | | | | | | A | |
| ATOM | 1972 | CB | PRO | 103 | 12.922 | −89.522 | 143.199 | 1.00 | 81.05 | B | C |
| ATOM | 1973 | CG | PRO | 103 | 13.639 | −89.140 | 144.492 | 1.00 | 81.13 | B | C |
| ATOM | 1974 | C | PRO | 103 | 13.827 | −89.814 | 140.872 | 1.00 | 80.76 | B | C |
| ATOM | 1975 | O | PRO | 103 | 13.218 | −88.817 | 140.479 | 1.00 | 80.92 | B | O |
| ATOM | 1976 | N | PRO | 103 | 15.298 | −89.351 | 142.841 | 1.00 | 81.13 | B | N |
| ATOM | 1977 | CD | PRO | 103 | 14.976 | −88.538 | 144.028 | 1.00 | 81.18 | B | C |
| ATOM | 1978 | CA | PRO | 103 | 14.080 | −90.046 | 142.362 | 1.00 | 80.95 | B | C |
| ATOM | 1979 | N | VAL | 104 | 14.299 | −90.742 | 140.047 | 1.00 | 80.28 | B | N |
| ATOM | 1980 | CA | VAL | 104 | 14.125 | −90.648 | 138.601 | 1.00 | 79.68 | B | C |
| ATOM | 1981 | CB | VAL | 104 | 15.488 | −90.651 | 137.868 | 1.00 | 79.97 | B | C |
| ATOM | 1982 | CG1 | VAL | 104 | 16.297 | −89.424 | 138.267 | 1.00 | 80.04 | B | C |
| ATOM | 1983 | CG2 | VAL | 104 | 16.254 | −91.933 | 138.186 | 1.00 | 79.80 | B | C |
| ATOM | 1984 | C | VAL | 104 | 13.296 | −91.823 | 138.096 | 1.00 | 78.96 | B | C |
| ATOM | 1985 | O | VAL | 104 | 13.242 | −92.872 | 138.740 | 1.00 | 79.04 | B | O |
| ATOM | 1986 | N | GLN | 105 | 12.654 | −91.649 | 136.943 | 1.00 | 77.77 | B | N |
| ATOM | 1987 | CA | GLN | 105 | 11.830 | −92.710 | 136.373 | 1.00 | 76.25 | B | C |
| ATOM | 1988 | CB | GLN | 105 | 10.461 | −92.159 | 135.952 | 1.00 | 76.99 | B | C |
| ATOM | 1989 | CG | GLN | 105 | 9.447 | −93.249 | 135.604 | 1.00 | 77.64 | B | C |
| ATOM | 1990 | CD | GLN | 105 | 8.032 | −92.718 | 135.433 | 1.00 | 78.06 | B | C |
| ATOM | 1991 | OE1 | GLN | 105 | 7.534 | −91.960 | 136.268 | 1.00 | 78.05 | B | O |
| ATOM | 1992 | NE2 | GLN | 105 | 7.371 | −93.130 | 134.355 | 1.00 | 77.95 | B | N |
| ATOM | 1993 | C | GLN | 105 | 12.495 | −93.407 | 135.185 | 1.00 | 74.64 | B | C |
| ATOM | 1994 | O | GLN | 105 | 12.485 | −94.634 | 135.110 | 1.00 | 74.66 | B | O |
| ATOM | 1995 | N | LEU | 106 | 13.067 | −92.618 | 134.273 | 1.00 | 72.54 | B | N |
| ATOM | 1996 | CA | LEU | 106 | 13.747 | −93.115 | 133.070 | 1.00 | 70.12 | B | C |
| ATOM | 1997 | CB | LEU | 106 | 15.259 | −92.860 | 133.160 | 1.00 | 70.25 | B | C |
| ATOM | 1998 | CG | LEU | 106 | 15.813 | −91.432 | 133.205 | 1.00 | 70.18 | B | C |
| ATOM | 1999 | CD1 | LEU | 106 | 15.723 | −90.881 | 134.616 | 1.00 | 70.47 | B | C |
| ATOM | 2000 | CD2 | LEU | 106 | 17.267 | −91.442 | 132.752 | 1.00 | 69.72 | B | C |
| ATOM | 2001 | C | LEU | 106 | 13.521 | −94.596 | 132.761 | 1.00 | 68.42 | B | C |
| ATOM | 2002 | O | LEU | 106 | 14.449 | −95.402 | 132.853 | 1.00 | 68.15 | B | O |
| ATOM | 2003 | N | SER | 107 | 12.295 | −94.949 | 132.382 | 1.00 | 66.23 | B | N |
| ATOM | 2004 | CA | SER | 107 | 11.955 | −96.333 | 132.064 | 1.00 | 63.76 | B | C |
| ATOM | 2005 | CB | SER | 107 | 10.469 | −96.441 | 131.721 | 1.00 | 63.69 | B | C |
| ATOM | 2006 | OG | SER | 107 | 10.152 | −97.713 | 131.185 | 1.00 | 63.42 | B | O |
| ATOM | 2007 | C | SER | 107 | 12.782 | −96.865 | 130.904 | 1.00 | 62.28 | B | C |
| ATOM | 2008 | O | SER | 107 | 13.328 | −96.097 | 130.117 | 1.00 | 62.13 | B | O |
| ATOM | 2009 | N | LYS | 108 | 12.878 | −98.187 | 130.806 | 1.00 | 60.42 | B | N |
| ATOM | 2010 | CA | LYS | 108 | 13.633 | −98.817 | 129.733 | 1.00 | 58.34 | B | C |
| ATOM | 2011 | CB | LYS | 108 | 13.706 | −100.328 | 129.957 | 1.00 | 59.09 | B | C |
| ATOM | 2012 | CG | LYS | 108 | 14.716 | −101.043 | 129.078 | 1.00 | 59.82 | B | C |
| ATOM | 2013 | CD | LYS | 108 | 16.139 | −100.630 | 129.430 | 1.00 | 61.01 | B | C |
| ATOM | 2014 | CE | LYS | 108 | 17.167 | −101.382 | 128.589 | 1.00 | 61.73 | B | C |
| ATOM | 2015 | NZ | LYS | 108 | 18.572 | −101.025 | 128.958 | 1.00 | 62.47 | B | N |
| ATOM | 2016 | C | LYS | 108 | 12.929 | −98.524 | 128.415 | 1.00 | 56.63 | B | C |
| ATOM | 2017 | O | LYS | 108 | 13.524 | −97.970 | 127.491 | 1.00 | 56.15 | B | O |
| ATOM | 2018 | N | GLU | 109 | 11.654 | −98.895 | 128.342 | 1.00 | 54.51 | B | N |
| ATOM | 2019 | CA | GLU | 109 | 10.846 | −98.673 | 127.148 | 1.00 | 52.68 | B | C |
| ATOM | 2020 | CB | GLU | 109 | 9.464 | −99.323 | 127.297 | 1.00 | 53.23 | B | C |
| ATOM | 2021 | CG | GLU | 109 | 9.004 | −99.560 | 128.730 | 1.00 | 54.59 | B | C |
| ATOM | 2022 | CD | GLU | 109 | 9.624 | −100.809 | 129.340 | 1.00 | 55.04 | B | C |
| ATOM | 2023 | OE1 | GLU | 109 | 9.379 | −101.914 | 128.809 | 1.00 | 55.28 | B | O |
| ATOM | 2024 | OE2 | GLU | 109 | 10.359 | −100.685 | 130.343 | 1.00 | 55.07 | B | O |
| ATOM | 2025 | C | GLU | 109 | 10.684 | −97.191 | 126.819 | 1.00 | 50.98 | B | C |
| ATOM | 2026 | O | GLU | 109 | 10.487 | −96.832 | 125.662 | 1.00 | 50.38 | B | O |
| ATOM | 2027 | N | GLN | 110 | 10.755 | −96.333 | 127.832 | 1.00 | 49.17 | B | N |
| ATOM | 2028 | CA | GLN | 110 | 10.636 | −94.901 | 127.607 | 1.00 | 47.33 | B | C |
| ATOM | 2029 | CB | GLN | 110 | 10.418 | −94.156 | 128.926 | 1.00 | 47.12 | B | C |
| ATOM | 2030 | CG | GLN | 110 | 9.089 | −94.471 | 129.606 | 1.00 | 47.16 | B | C |
| ATOM | 2031 | CD | GLN | 110 | 8.874 | −93.656 | 130.870 | 1.00 | 47.26 | B | C |
| ATOM | 2032 | OE1 | GLN | 110 | 9.767 | −93.545 | 131.711 | 1.00 | 46.64 | B | O |
| ATOM | 2033 | NE2 | GLN | 110 | 7.682 | −93.086 | 131.014 | 1.00 | 47.72 | B | N |
| ATOM | 2034 | C | GLN | 110 | 11.896 | −94.386 | 126.921 | 1.00 | 46.46 | B | C |
| ATOM | 2035 | O | GLN | 110 | 11.815 | −93.558 | 126.018 | 1.00 | 45.75 | B | O |
| ATOM | 2036 | N | GLU | 111 | 13.061 | −94.871 | 127.343 | 1.00 | 45.39 | B | N |
| ATOM | 2037 | CA | GLU | 111 | 14.306 | −94.441 | 126.714 | 1.00 | 44.94 | B | C |
| ATOM | 2038 | CB | GLU | 111 | 15.526 | −95.026 | 127.436 | 1.00 | 46.33 | B | C |
| ATOM | 2039 | CG | GLU | 111 | 15.591 | −94.724 | 128.924 | 1.00 | 48.97 | B | C |
| ATOM | 2040 | CD | GLU | 111 | 17.005 | −94.789 | 129.479 | 1.00 | 50.99 | B | C |
| ATOM | 2041 | OE1 | GLU | 111 | 17.745 | −93.789 | 129.335 | 1.00 | 51.59 | B | O |
| ATOM | 2042 | OE2 | GLU | 111 | 17.378 | −95.840 | 130.049 | 1.00 | 51.77 | B | O |
| ATOM | 2043 | C | GLU | 111 | 14.292 | −94.918 | 125.262 | 1.00 | 43.27 | B | C |
| ATOM | 2044 | O | GLU | 111 | 14.768 | −94.227 | 124.362 | 1.00 | 42.84 | B | O |
| ATOM | 2045 | N | GLU | 112 | 13.735 | −96.107 | 125.050 | 1.00 | 41.39 | B | N |
| ATOM | 2046 | CA | GLU | 112 | 13.627 | −96.696 | 123.724 | 1.00 | 39.73 | B | C |
| ATOM | 2047 | CB | GLU | 112 | 13.040 | −98.104 | 123.833 | 1.00 | 40.89 | B | C |
| ATOM | 2048 | CG | GLU | 112 | 12.809 | −98.801 | 122.500 | 1.00 | 43.06 | B | C |
| ATOM | 2049 | CD | GLU | 112 | 14.047 | −98.808 | 121.624 | 1.00 | 44.55 | B | C |
| ATOM | 2050 | OE1 | GLU | 112 | 15.139 | −99.130 | 122.139 | 1.00 | 45.11 | B | O |
| ATOM | 2051 | OE2 | GLU | 112 | 13.927 | −98.497 | 120.419 | 1.00 | 46.10 | B | O |
| ATOM | 2052 | C | GLU | 112 | 12.733 | −95.825 | 122.844 | 1.00 | 38.09 | B | C |
| ATOM | 2053 | O | GLU | 112 | 13.030 | −95.594 | 121.666 | 1.00 | 37.26 | B | O |
| ATOM | 2054 | N | LEU | 113 | 11.638 | −95.349 | 123.430 | 1.00 | 35.31 | B | N |
| ATOM | 2055 | CA | LEU | 113 | 10.691 | −94.500 | 122.729 | 1.00 | 33.09 | B | C |
| ATOM | 2056 | CB | LEU | 113 | 9.499 | −94.178 | 123.637 | 1.00 | 32.27 | B | C |
| ATOM | 2057 | CG | LEU | 113 | 8.480 | −93.165 | 123.101 | 1.00 | 32.57 | B | C |
| ATOM | 2058 | CD1 | LEU | 113 | 7.983 | −93.617 | 121.731 | 1.00 | 31.91 | B | C |
| ATOM | 2059 | CD2 | LEU | 113 | 7.325 | −93.014 | 124.080 | 1.00 | 31.00 | B | C |
| ATOM | 2060 | C | LEU | 113 | 11.380 | −93.213 | 122.294 | 1.00 | 31.56 | B | C |
| ATOM | 2061 | O | LEU | 113 | 11.268 | −92.799 | 121.138 | 1.00 | 30.78 | B | O |
| ATOM | 2062 | N | ILE | 114 | 12.089 | −92.586 | 123.227 | 1.00 | 30.06 | B | N |
| ATOM | 2063 | CA | ILE | 114 | 12.808 | −91.351 | 122.949 | 1.00 | 29.43 | B | C |
| ATOM | 2064 | CB | ILE | 114 | 13.518 | −90.822 | 124.221 | 1.00 | 28.14 | B | C |
| ATOM | 2065 | CG2 | ILE | 114 | 14.463 | −89.686 | 123.870 | 1.00 | 27.33 | B | C |
| ATOM | 2066 | CG1 | ILE | 114 | 12.472 | −90.330 | 125.228 | 1.00 | 27.74 | B | C |
| ATOM | 2067 | CD1 | ILE | 114 | 13.058 | −89.868 | 126.541 | 1.00 | 26.41 | B | C |
| ATOM | 2068 | C | ILE | 114 | 13.837 | −91.546 | 121.836 | 1.00 | 29.65 | B | C |
| ATOM | 2069 | O | ILE | 114 | 13.866 | −90.789 | 120.872 | 1.00 | 28.66 | B | O |
| ATOM | 2070 | N | ARG | 115 | 14.672 | −92.571 | 121.961 | 1.00 | 30.65 | B | N |
| ATOM | 2071 | CA | ARG | 115 | 15.686 | −92.821 | 120.947 | 1.00 | 32.09 | B | C |
| ATOM | 2072 | CB | ARG | 115 | 16.540 | −94.037 | 121.319 | 1.00 | 34.79 | B | C |
| ATOM | 2073 | CG | ARG | 115 | 17.947 | −93.967 | 120.729 | 1.00 | 39.78 | B | C |
| ATOM | 2074 | CD | ARG | 115 | 18.821 | −95.166 | 121.077 | 1.00 | 43.64 | B | C |
| ATOM | 2075 | NE | ARG | 115 | 18.482 | −96.354 | 120.292 | 1.00 | 47.79 | B | N |
| ATOM | 2076 | CZ | ARG | 115 | 17.535 | −97.233 | 120.612 | 1.00 | 49.59 | B | C |
| ATOM | 2077 | NH1 | ARG | 115 | 16.815 | −97.074 | 121.713 | 1.00 | 51.59 | B | N |
| ATOM | 2078 | NH2 | ARG | 115 | 17.311 | −98.281 | 119.832 | 1.00 | 50.36 | B | N |
| ATOM | 2079 | C | ARG | 115 | 15.069 | −93.016 | 119.562 | 1.00 | 30.91 | B | C |
| ATOM | 2080 | O | ARG | 115 | 15.599 | −92.516 | 118.571 | 1.00 | 31.00 | B | O |
| ATOM | 2081 | N | THR | 116 | 13.952 | −93.734 | 119.496 | 1.00 | 29.49 | B | N |
| ATOM | 2082 | CA | THR | 116 | 13.263 | −93.974 | 118.231 | 1.00 | 28.65 | B | C |
| ATOM | 2083 | CB | THR | 116 | 12.058 | −94.914 | 118.428 | 1.00 | 29.45 | B | C |
| ATOM | 2084 | OG1 | THR | 116 | 12.514 | −96.168 | 118.945 | 1.00 | 31.34 | B | O |
| ATOM | 2085 | CG2 | THR | 116 | 11.332 | −95.147 | 117.112 | 1.00 | 29.40 | B | C |
| ATOM | 2086 | C | THR | 116 | 12.757 | −92.658 | 117.640 | 1.00 | 27.59 | B | C |
| ATOM | 2087 | O | THR | 116 | 12.995 | −92.359 | 116.469 | 1.00 | 27.35 | B | O |
| ATOM | 2088 | N | LEU | 117 | 12.049 | −91.882 | 118.455 | 1.00 | 25.67 | B | N |
| ATOM | 2089 | CA | LEU | 117 | 11.517 | −90.594 | 118.019 | 1.00 | 24.51 | B | C |
| ATOM | 2090 | CB | LEU | 117 | 10.691 | −89.949 | 119.143 | 1.00 | 22.66 | B | C |
| ATOM | 2091 | CG | LEU | 117 | 9.309 | −90.544 | 119.437 | 1.00 | 23.35 | B | C |
| ATOM | 2092 | CD1 | LEU | 117 | 8.752 | −89.953 | 120.731 | 1.00 | 22.38 | B | C |
| ATOM | 2093 | CD2 | LEU | 117 | 8.362 | −90.261 | 118.270 | 1.00 | 22.82 | B | C |
| ATOM | 2094 | C | LEU | 117 | 12.647 | −89.657 | 117.607 | 1.00 | 22.88 | B | C |
| ATOM | 2095 | O | LEU | 117 | 12.566 | −88.996 | 116.580 | 1.00 | 22.11 | B | O |
| ATOM | 2096 | N | LEU | 118 | 13.698 | −89.608 | 118.416 | 1.00 | 23.09 | B | N |
| ATOM | 2097 | CA | LEU | 118 | 14.856 | −88.755 | 118.152 | 1.00 | 23.66 | B | C |
| ATOM | 2098 | CB | LEU | 118 | 15.879 | −88.903 | 119.276 | 1.00 | 25.11 | B | C |
| ATOM | 2099 | CG | LEU | 118 | 16.702 | −87.685 | 119.697 | 1.00 | 27.59 | B | C |
| ATOM | 2100 | CD1 | LEU | 118 | 18.037 | −88.188 | 120.242 | 1.00 | 28.18 | B | C |
| ATOM | 2101 | CD2 | LEU | 118 | 16.932 | −86.729 | 118.527 | 1.00 | 28.59 | B | C |
| ATOM | 2102 | C | LEU | 118 | 15.520 | −89.134 | 116.827 | 1.00 | 23.33 | B | C |
| ATOM | 2103 | O | LEU | 118 | 15.921 | −88.260 | 116.054 | 1.00 | 22.58 | B | O |
| ATOM | 2104 | N | GLY | 119 | 15.644 | −90.441 | 116.585 | 1.00 | 22.04 | B | N |
| ATOM | 2105 | CA | GLY | 119 | 16.255 | −90.931 | 115.362 | 1.00 | 21.87 | B | C |
| ATOM | 2106 | C | GLY | 119 | 15.491 | −90.497 | 114.127 | 1.00 | 21.81 | B | C |
| ATOM | 2107 | O | GLY | 119 | 16.072 | −89.949 | 113.191 | 1.00 | 21.52 | B | O |
| ATOM | 2108 | N | ALA | 120 | 14.185 | −90.743 | 114.122 | 1.00 | 21.37 | B | N |
| ATOM | 2109 | CA | ALA | 120 | 13.331 | −90.358 | 113.004 | 1.00 | 21.05 | B | C |
| ATOM | 2110 | CB | ALA | 120 | 11.913 | −90.885 | 113.233 | 1.00 | 20.81 | B | C |
| ATOM | 2111 | C | ALA | 120 | 13.304 | −88.829 | 112.833 | 1.00 | 21.40 | B | C |
| ATOM | 2112 | O | ALA | 120 | 13.372 | −88.314 | 111.716 | 1.00 | 20.48 | B | O |
| ATOM | 2113 | N | HIS | 121 | 13.191 | −88.110 | 113.945 | 1.00 | 22.14 | B | N |
| ATOM | 2114 | CA | HIS | 121 | 13.166 | −86.649 | 113.914 | 1.00 | 22.47 | B | C |
| ATOM | 2115 | CB | HIS | 121 | 12.936 | −86.096 | 115.325 | 1.00 | 22.23 | B | C |
| ATOM | 2116 | CG | HIS | 121 | 13.136 | −84.619 | 115.433 | 1.00 | 24.18 | B | C |
| ATOM | 2117 | CD2 | HIS | 121 | 12.269 | −83.589 | 115.284 | 1.00 | 23.87 | B | C |
| ATOM | 2118 | ND1 | HIS | 121 | 14.373 | −84.050 | 115.657 | 1.00 | 24.73 | B | N |
| ATOM | 2119 | CE1 | HIS | 121 | 14.258 | −82.734 | 115.638 | 1.00 | 25.53 | B | C |
| ATOM | 2120 | NE2 | HIS | 121 | 12.992 | −82.430 | 115.413 | 1.00 | 25.90 | B | N |
| ATOM | 2121 | C | HIS | 121 | 14.469 | −86.093 | 113.339 | 1.00 | 22.36 | B | C |
| ATOM | 2122 | O | HIS | 121 | 14.452 | −85.248 | 112.448 | 1.00 | 22.60 | B | O |
| ATOM | 2123 | N | THR | 122 | 15.597 | −86.574 | 113.849 | 1.00 | 21.59 | B | N |
| ATOM | 2124 | CA | THR | 122 | 16.900 | −86.119 | 113.387 | 1.00 | 21.97 | B | C |
| ATOM | 2125 | CB | THR | 122 | 18.038 | −86.782 | 114.199 | 1.00 | 22.09 | B | C |
| ATOM | 2126 | OG1 | THR | 122 | 17.948 | −86.364 | 115.561 | 1.00 | 24.17 | B | O |
| ATOM | 2127 | CG2 | THR | 122 | 19.397 | −86.384 | 113.655 | 1.00 | 22.21 | B | C |
| ATOM | 2128 | C | THR | 122 | 17.142 | −86.400 | 111.907 | 1.00 | 21.55 | B | C |
| ATOM | 2129 | O | THR | 122 | 17.664 | −85.549 | 111.188 | 1.00 | 20.90 | B | O |
| ATOM | 2130 | N | ARG | 123 | 16.773 | −87.595 | 111.455 | 1.00 | 21.32 | B | N |
| ATOM | 2131 | CA | ARG | 123 | 16.990 | −87.969 | 110.063 | 1.00 | 22.23 | B | C |
| ATOM | 2132 | CB | ARG | 123 | 16.731 | −89.472 | 109.852 | 1.00 | 21.90 | B | C |
| ATOM | 2133 | CG | ARG | 123 | 17.899 | −90.405 | 110.237 | 1.00 | 22.07 | B | C |
| ATOM | 2134 | CD | ARG | 123 | 17.662 | −91.846 | 109.753 | 1.00 | 20.33 | B | C |
| ATOM | 2135 | NE | ARG | 123 | 16.487 | −92.450 | 110.380 | 1.00 | 21.86 | B | N |
| ATOM | 2136 | CZ | ARG | 123 | 16.465 | −92.979 | 111.601 | 1.00 | 21.57 | B | C |
| ATOM | 2137 | NH1 | ARG | 123 | 17.559 | −93.002 | 112.352 | 1.00 | 21.63 | B | N |
| ATOM | 2138 | NH2 | ARG | 123 | 15.333 | −93.467 | 112.086 | 1.00 | 20.21 | B | N |
| ATOM | 2139 | C | ARG | 123 | 16.166 | −87.193 | 109.043 | 1.00 | 22.64 | B | C |
| ATOM | 2140 | O | ARG | 123 | 16.664 | −86.867 | 107.971 | 1.00 | 23.15 | B | O |
| ATOM | 2141 | N | HIS | 124 | 14.918 | −86.877 | 109.370 | 1.00 | 22.90 | B | N |
| ATOM | 2142 | CA | HIS | 124 | 14.061 | −86.204 | 108.398 | 1.00 | 23.53 | B | C |
| ATOM | 2143 | CB | HIS | 124 | 12.841 | −87.089 | 108.121 | 1.00 | 22.74 | B | C |
| ATOM | 2144 | CG | HIS | 124 | 13.190 | −88.501 | 107.763 | 1.00 | 23.34 | B | C |
| ATOM | 2145 | CD2 | HIS | 124 | 13.757 | −89.023 | 106.650 | 1.00 | 22.54 | B | C |
| ATOM | 2146 | ND1 | HIS | 124 | 12.991 | −89.562 | 108.624 | 1.00 | 22.99 | B | N |
| ATOM | 2147 | CE1 | HIS | 124 | 13.421 | −90.674 | 108.055 | 1.00 | 21.00 | B | C |
| ATOM | 2148 | NE2 | HIS | 124 | 13.891 | −90.375 | 106.858 | 1.00 | 21.93 | B | N |
| ATOM | 2149 | C | HIS | 124 | 13.588 | −84.772 | 108.653 | 1.00 | 23.63 | B | C |
| ATOM | 2150 | O | HIS | 124 | 13.238 | −84.069 | 107.700 | 1.00 | 24.39 | B | O |
| ATOM | 2151 | N | MET | 125 | 13.589 | −84.322 | 109.905 | 1.00 | 22.90 | B | N |
| ATOM | 2152 | CA | MET | 125 | 13.097 | −82.976 | 110.197 | 1.00 | 22.76 | B | C |
| ATOM | 2153 | CB | MET | 125 | 11.817 | −83.071 | 111.038 | 1.00 | 22.21 | B | C |
| ATOM | 2154 | CG | MET | 125 | 10.710 | −83.920 | 110.415 | 1.00 | 21.83 | B | C |
| ATOM | 2155 | SD | MET | 125 | 9.120 | −83.745 | 111.269 | 1.00 | 22.85 | B | S |
| ATOM | 2156 | CE | MET | 125 | 9.499 | −84.456 | 112.900 | 1.00 | 22.30 | B | C |
| ATOM | 2157 | C | MET | 125 | 14.065 | −82.015 | 110.880 | 1.00 | 22.78 | B | C |
| ATOM | 2158 | O | MET | 125 | 14.118 | −80.833 | 110.530 | 1.00 | 22.22 | B | O |
| ATOM | 2159 | N | GLY | 126 | 14.818 | −82.527 | 111.852 | 1.00 | 22.55 | B | N |
| ATOM | 2160 | CA | GLY | 126 | 15.758 | −81.721 | 112.614 | 1.00 | 22.45 | B | C |
| ATOM | 2161 | C | GLY | 126 | 16.466 | −80.574 | 111.919 | 1.00 | 23.15 | B | C |
| ATOM | 2162 | O | GLY | 126 | 16.502 | −79.458 | 112.438 | 1.00 | 23.04 | B | O |
| ATOM | 2163 | N | THR | 127 | 17.047 | −80.840 | 110.757 | 1.00 | 22.96 | B | N |
| ATOM | 2164 | CA | THR | 127 | 17.756 | −79.804 | 110.026 | 1.00 | 23.87 | B | C |
| ATOM | 2165 | CB | THR | 127 | 19.261 | −80.133 | 109.920 | 1.00 | 25.68 | B | C |
| ATOM | 2166 | OG1 | THR | 127 | 19.417 | −81.510 | 109.569 | 1.00 | 27.25 | B | O |
| ATOM | 2167 | CG2 | THR | 127 | 19.969 | −79.868 | 111.242 | 1.00 | 26.03 | B | C |
| ATOM | 2168 | C | THR | 127 | 17.203 | −79.606 | 108.624 | 1.00 | 22.94 | B | C |
| ATOM | 2169 | O | THR | 127 | 17.920 | −79.166 | 107.728 | 1.00 | 22.72 | B | O |
| ATOM | 2170 | N | MET | 128 | 15.927 | −79.925 | 108.429 | 1.00 | 22.41 | B | N |
| ATOM | 2171 | CA | MET | 128 | 15.320 | −79.764 | 107.114 | 1.00 | 22.16 | B | C |
| ATOM | 2172 | CB | MET | 128 | 13.897 | −80.330 | 107.101 | 1.00 | 21.38 | B | C |
| ATOM | 2173 | CG | MET | 128 | 12.872 | −79.579 | 107.943 | 1.00 | 20.15 | B | C |
| ATOM | 2174 | SD | MET | 128 | 11.239 | −80.338 | 107.749 | 1.00 | 20.67 | B | S |
| ATOM | 2175 | CE | MET | 128 | 10.284 | −79.362 | 108.917 | 1.00 | 22.56 | B | C |
| ATOM | 2176 | C | MET | 128 | 15.305 | −78.305 | 106.669 | 1.00 | 22.32 | B | C |
| ATOM | 2177 | O | MET | 128 | 15.261 | −78.019 | 105.476 | 1.00 | 22.98 | B | O |
| ATOM | 2178 | N | PHE | 129 | 15.363 | −77.384 | 107.627 | 1.00 | 22.69 | B | N |
| ATOM | 2179 | CA | PHE | 129 | 15.358 | −75.957 | 107.311 | 1.00 | 24.34 | B | C |
| ATOM | 2180 | CB | PHE | 129 | 15.281 | −75.127 | 108.605 | 1.00 | 25.41 | B | C |
| ATOM | 2181 | CG | PHE | 129 | 16.565 | −75.092 | 109.396 | 1.00 | 28.48 | B | C |
| ATOM | 2182 | CD1 | PHE | 129 | 17.588 | −74.210 | 109.052 | 1.00 | 30.09 | B | C |
| ATOM | 2183 | CD2 | PHE | 129 | 16.749 | −75.934 | 110.486 | 1.00 | 29.53 | B | C |
| ATOM | 2184 | CE1 | PHE | 129 | 18.775 | −74.164 | 109.784 | 1.00 | 31.38 | B | C |
| ATOM | 2185 | CE2 | PHE | 129 | 17.928 | −75.900 | 111.228 | 1.00 | 31.21 | B | C |
| ATOM | 2186 | CZ | PHE | 129 | 18.945 | −75.012 | 110.875 | 1.00 | 32.22 | B | C |
| ATOM | 2187 | C | PHE | 129 | 16.580 | −75.540 | 106.485 | 1.00 | 24.09 | B | C |
| ATOM | 2188 | O | PHE | 129 | 16.566 | −74.501 | 105.821 | 1.00 | 23.81 | B | O |
| ATOM | 2189 | N | GLU | 130 | 17.636 | −76.346 | 106.522 | 1.00 | 24.21 | B | N |
| ATOM | 2190 | CA | GLU | 130 | 18.843 | −76.031 | 105.762 | 1.00 | 25.33 | B | C |
| ATOM | 2191 | CB | GLU | 130 | 20.011 | −76.906 | 106.222 | 1.00 | 27.28 | B | C |
| ATOM | 2192 | CG | GLU | 130 | 20.376 | −76.718 | 107.685 | 1.00 | 31.35 | B | C |
| ATOM | 2193 | CD | GLU | 130 | 21.694 | −77.377 | 108.045 | 1.00 | 34.28 | B | C |
| ATOM | 2194 | OE1 | GLU | 130 | 22.057 | −78.381 | 107.389 | 1.00 | 35.08 | B | O |
| ATOM | 2195 | OE2 | GLU | 130 | 22.360 | −76.898 | 108.992 | 1.00 | 36.90 | B | O |
| ATOM | 2196 | C | GLU | 130 | 18.647 | −76.197 | 104.258 | 1.00 | 23.24 | B | C |
| ATOM | 2197 | O | GLU | 130 | 19.439 | −75.686 | 103.470 | 1.00 | 23.56 | B | O |
| ATOM | 2198 | N | GLN | 131 | 17.601 | −76.918 | 103.865 | 1.00 | 21.55 | B | N |
| ATOM | 2199 | CA | GLN | 131 | 17.302 | −77.125 | 102.453 | 1.00 | 20.76 | B | C |
| ATOM | 2200 | CB | GLN | 131 | 16.539 | −78.442 | 102.240 | 1.00 | 21.85 | B | C |
| ATOM | 2201 | CG | GLN | 131 | 17.320 | −79.703 | 102.536 | 1.00 | 23.34 | B | C |
| ATOM | 2202 | CD | GLN | 131 | 18.691 | −79.696 | 101.882 | 1.00 | 26.07 | B | C |
| ATOM | 2203 | OE1 | GLN | 131 | 18.815 | −79.538 | 100.664 | 1.00 | 26.66 | B | O |
| ATOM | 2204 | NE2 | GLN | 131 | 19.728 | −79.862 | 102.692 | 1.00 | 26.55 | B | N |
| ATOM | 2205 | C | GLN | 131 | 16.462 | −75.987 | 101.869 | 1.00 | 20.02 | B | C |
| ATOM | 2206 | O | GLN | 131 | 16.346 | −75.875 | 100.659 | 1.00 | 20.04 | B | O |
| ATOM | 2207 | N | PHE | 132 | 15.880 | −75.149 | 102.724 | 1.00 | 19.42 | B | N |
| ATOM | 2208 | CA | PHE | 132 | 15.023 | −74.048 | 102.269 | 1.00 | 19.05 | B | C |
| ATOM | 2209 | CB | PHE | 132 | 14.612 | −73.157 | 103.454 | 1.00 | 17.06 | B | C |
| ATOM | 2210 | CG | PHE | 132 | 13.572 | −73.779 | 104.384 | 1.00 | 16.44 | B | C |
| ATOM | 2211 | CD1 | PHE | 132 | 13.055 | −75.055 | 104.152 | 1.00 | 15.33 | B | C |
| ATOM | 2212 | CD2 | PHE | 132 | 13.117 | −73.073 | 105.493 | 1.00 | 15.55 | B | C |
| ATOM | 2213 | CE1 | PHE | 132 | 12.099 | −75.620 | 105.018 | 1.00 | 15.93 | B | C |
| ATOM | 2214 | CE2 | PHE | 132 | 12.157 | −73.626 | 106.368 | 1.00 | 15.23 | B | C |
| ATOM | 2215 | CZ | PHE | 132 | 11.651 | −74.895 | 106.132 | 1.00 | 15.15 | B | C |
| ATOM | 2216 | C | PHE | 132 | 15.650 | −73.182 | 101.170 | 1.00 | 19.75 | B | C |
| ATOM | 2217 | O | PHE | 132 | 14.957 | −72.736 | 100.254 | 1.00 | 19.12 | B | O |
| ATOM | 2218 | N | VAL | 133 | 16.955 | −72.946 | 101.266 | 1.00 | 20.25 | B | N |
| ATOM | 2219 | CA | VAL | 133 | 17.675 | −72.139 | 100.287 | 1.00 | 21.53 | B | C |
| ATOM | 2220 | CB | VAL | 133 | 19.135 | −71.915 | 100.737 | 1.00 | 22.78 | B | C |
| ATOM | 2221 | CG1 | VAL | 133 | 19.901 | −73.236 | 100.711 | 1.00 | 21.70 | B | C |
| ATOM | 2222 | CG2 | VAL | 133 | 19.799 | −70.870 | 99.850 | 1.00 | 23.73 | B | C |
| ATOM | 2223 | C | VAL | 133 | 17.673 | −72.750 | 98.877 | 1.00 | 22.37 | B | C |
| ATOM | 2224 | O | VAL | 133 | 17.955 | −72.058 | 97.895 | 1.00 | 21.91 | B | O |
| ATOM | 2225 | N | GLN | 134 | 17.344 | −74.036 | 98.780 | 1.00 | 22.29 | B | N |
| ATOM | 2226 | CA | GLN | 134 | 17.294 | −74.725 | 97.496 | 1.00 | 23.37 | B | C |
| ATOM | 2227 | CB | GLN | 134 | 17.558 | −76.229 | 97.680 | 1.00 | 22.92 | B | C |
| ATOM | 2228 | CG | GLN | 134 | 18.860 | −76.621 | 98.407 | 1.00 | 23.40 | B | C |
| ATOM | 2229 | CD | GLN | 134 | 20.154 | −76.250 | 97.659 | 1.00 | 23.05 | B | C |
| ATOM | 2230 | OE1 | GLN | 134 | 21.237 | −76.701 | 98.020 | 1.00 | 25.27 | B | O |
| ATOM | 2231 | NE2 | GLN | 134 | 20.042 | −75.430 | 96.639 | 1.00 | 22.55 | B | N |
| ATOM | 2232 | C | GLN | 134 | 15.954 | −74.548 | 96.768 | 1.00 | 24.55 | B | C |
| ATOM | 2233 | O | GLN | 134 | 15.771 | −75.081 | 95.678 | 1.00 | 24.39 | B | O |
| ATOM | 2234 | N | PHE | 135 | 15.022 | −73.804 | 97.361 | 1.00 | 24.64 | B | N |
| ATOM | 2235 | CA | PHE | 135 | 13.710 | −73.595 | 96.750 | 1.00 | 25.44 | B | C |
| ATOM | 2236 | CB | PHE | 135 | 12.626 | −74.065 | 97.722 | 1.00 | 24.79 | B | C |
| ATOM | 2237 | CG | PHE | 135 | 12.706 | −75.534 | 98.039 | 1.00 | 23.89 | B | C |
| ATOM | 2238 | CD1 | PHE | 135 | 12.206 | −76.478 | 97.146 | 1.00 | 24.40 | B | C |
| ATOM | 2239 | CD2 | PHE | 135 | 13.335 | −75.977 | 99.198 | 1.00 | 24.10 | B | C |
| ATOM | 2240 | CE1 | PHE | 135 | 12.331 | −77.848 | 97.397 | 1.00 | 24.28 | B | C |
| ATOM | 2241 | CE2 | PHE | 135 | 13.470 | −77.349 | 99.466 | 1.00 | 24.09 | B | C |
| ATOM | 2242 | CZ | PHE | 135 | 12.966 | −78.286 | 98.560 | 1.00 | 24.17 | B | C |
| ATOM | 2243 | C | PHE | 135 | 13.500 | −72.130 | 96.358 | 1.00 | 27.05 | B | C |
| ATOM | 2244 | O | PHE | 135 | 12.508 | −71.501 | 96.739 | 1.00 | 27.09 | B | O |
| ATOM | 2245 | N | ARG | 136 | 14.444 | −71.622 | 95.566 | 1.00 | 27.76 | B | N |
| ATOM | 2246 | CA | ARG | 136 | 14.486 | −70.241 | 95.098 | 1.00 | 28.77 | B | C |
| ATOM | 2247 | CB | ARG | 136 | 14.046 | −70.118 | 93.626 | 1.00 | 30.48 | B | C |
| ATOM | 2248 | CG | ARG | 136 | 12.754 | −70.801 | 93.245 | 1.00 | 34.79 | B | C |
| ATOM | 2249 | CD | ARG | 136 | 13.002 | −72.190 | 92.685 | 1.00 | 36.21 | B | C |
| ATOM | 2250 | NE | ARG | 136 | 11.998 | −73.127 | 93.184 | 1.00 | 39.78 | B | N |
| ATOM | 2251 | CZ | ARG | 136 | 12.120 | −74.448 | 93.158 | 1.00 | 40.31 | B | C |
| ATOM | 2252 | NH1 | ARG | 136 | 13.214 | −75.011 | 92.649 | 1.00 | 41.22 | B | N |
| ATOM | 2253 | NH2 | ARG | 136 | 11.152 | −75.207 | 93.654 | 1.00 | 40.63 | B | N |
| ATOM | 2254 | C | ARG | 136 | 13.765 | −69.204 | 95.951 | 1.00 | 28.40 | B | C |
| ATOM | 2255 | O | ARG | 136 | 12.679 | −68.725 | 95.618 | 1.00 | 27.33 | B | O |
| ATOM | 2256 | N | PRO | 137 | 14.379 | −68.850 | 97.085 | 1.00 | 28.01 | B | N |
| ATOM | 2257 | CD | PRO | 137 | 15.580 | −69.475 | 97.667 | 1.00 | 27.43 | B | C |
| ATOM | 2258 | CA | PRO | 137 | 13.821 | −67.860 | 97.999 | 1.00 | 27.65 | B | C |
| ATOM | 2259 | CB | PRO | 137 | 14.586 | −68.120 | 99.286 | 1.00 | 27.04 | B | C |
| ATOM | 2260 | CG | PRO | 137 | 15.924 | −68.518 | 98.778 | 1.00 | 27.95 | B | C |
| ATOM | 2261 | C | PRO | 137 | 14.108 | −66.467 | 97.451 | 1.00 | 27.46 | B | C |
| ATOM | 2262 | O | PRO | 137 | 15.212 | −66.190 | 96.981 | 1.00 | 27.51 | B | O |
| ATOM | 2263 | N | PRO | 138 | 13.110 | −65.578 | 97.479 | 1.00 | 26.95 | B | N |
| ATOM | 2264 | CD | PRO | 138 | 11.696 | −65.734 | 97.852 | 1.00 | 26.96 | B | C |
| ATOM | 2265 | CA | PRO | 138 | 13.372 | −64.234 | 96.968 | 1.00 | 26.24 | B | C |
| ATOM | 2266 | CB | PRO | 138 | 12.051 | −63.496 | 97.228 | 1.00 | 26.54 | B | C |
| ATOM | 2267 | CG | PRO | 138 | 11.350 | −64.341 | 98.273 | 1.00 | 27.40 | B | C |
| ATOM | 2268 | C | PRO | 138 | 14.573 | −63.608 | 97.679 | 1.00 | 25.08 | B | C |
| ATOM | 2269 | O | PRO | 138 | 14.928 | −63.992 | 98.799 | 1.00 | 23.40 | B | O |
| ATOM | 2270 | N | ALA | 139 | 15.194 | −62.642 | 97.014 | 1.00 | 24.45 | B | N |
| ATOM | 2271 | CA | ALA | 139 | 16.377 | −61.959 | 97.529 | 1.00 | 24.74 | B | C |
| ATOM | 2272 | CB | ALA | 139 | 16.852 | −60.925 | 96.503 | 1.00 | 25.14 | B | C |
| ATOM | 2273 | C | ALA | 139 | 16.283 | −61.300 | 98.905 | 1.00 | 24.49 | B | C |
| ATOM | 2274 | O | ALA | 139 | 17.268 | −61.293 | 99.651 | 1.00 | 24.23 | B | O |
| ATOM | 2275 | N | HIS | 140 | 15.120 | −60.749 | 99.252 | 1.00 | 24.55 | B | N |
| ATOM | 2276 | CA | HIS | 140 | 14.979 | −60.066 | 100.543 | 1.00 | 24.29 | B | C |
| ATOM | 2277 | CB | HIS | 140 | 13.677 | −59.245 | 100.587 | 1.00 | 23.87 | B | C |
| ATOM | 2278 | CG | HIS | 140 | 12.440 | −60.056 | 100.826 | 1.00 | 23.38 | B | C |
| ATOM | 2279 | CD2 | HIS | 140 | 11.696 | −60.232 | 101.944 | 1.00 | 23.33 | B | C |
| ATOM | 2280 | ND1 | HIS | 140 | 11.817 | −60.783 | 99.833 | 1.00 | 23.88 | B | N |
| ATOM | 2281 | CE1 | HIS | 140 | 10.743 | −61.371 | 100.328 | 1.00 | 24.02 | B | C |
| ATOM | 2282 | NE2 | HIS | 140 | 10.647 | −61.054 | 101.608 | 1.00 | 24.40 | B | N |
| ATOM | 2283 | C | HIS | 140 | 15.080 | −60.951 | 101.790 | 1.00 | 24.34 | B | C |
| ATOM | 2284 | O | HIS | 140 | 15.223 | −60.439 | 102.901 | 1.00 | 24.05 | B | O |
| ATOM | 2285 | N | LEU | 141 | 15.010 | −62.265 | 101.605 | 1.00 | 24.45 | B | N |
| ATOM | 2286 | CA | LEU | 141 | 15.117 | −63.216 | 102.714 | 1.00 | 25.64 | B | C |
| ATOM | 2287 | CB | LEU | 141 | 14.436 | −64.544 | 102.352 | 1.00 | 24.52 | B | C |
| ATOM | 2288 | CG | LEU | 141 | 12.951 | −64.562 | 101.981 | 1.00 | 25.14 | B | C |
| ATOM | 2289 | CD1 | LEU | 141 | 12.529 | −65.992 | 101.647 | 1.00 | 23.52 | B | C |
| ATOM | 2290 | CD2 | LEU | 141 | 12.118 | −64.006 | 103.143 | 1.00 | 23.73 | B | C |
| ATOM | 2291 | C | LEU | 141 | 16.577 | −63.512 | 103.056 | 1.00 | 26.62 | B | C |
| ATOM | 2292 | O | LEU | 141 | 16.886 | −64.005 | 104.138 | 1.00 | 26.38 | B | O |
| ATOM | 2293 | N | PHE | 142 | 17.476 | −63.213 | 102.130 | 1.00 | 28.49 | B | N |
| ATOM | 2294 | CA | PHE | 142 | 18.888 | −63.495 | 102.331 | 1.00 | 31.82 | B | C |
| ATOM | 2295 | CB | PHE | 142 | 19.611 | −63.519 | 100.985 | 1.00 | 29.28 | B | C |
| ATOM | 2296 | CG | PHE | 142 | 19.392 | −64.779 | 100.212 | 1.00 | 27.30 | B | C |
| ATOM | 2297 | CD1 | PHE | 142 | 20.232 | −65.873 | 100.393 | 1.00 | 25.11 | B | C |
| ATOM | 2298 | CD2 | PHE | 142 | 18.323 | −64.890 | 99.331 | 1.00 | 26.50 | B | C |
| ATOM | 2299 | CE1 | PHE | 142 | 20.010 | −67.056 | 99.706 | 1.00 | 25.73 | B | C |
| ATOM | 2300 | CE2 | PHE | 142 | 18.093 | −66.074 | 98.639 | 1.00 | 26.27 | B | C |
| ATOM | 2301 | CZ | PHE | 142 | 18.937 | −67.159 | 98.826 | 1.00 | 25.25 | B | C |
| ATOM | 2302 | C | PHE | 142 | 19.648 | −62.591 | 103.275 | 1.00 | 35.28 | B | C |
| ATOM | 2303 | O | PHE | 142 | 19.462 | −61.377 | 103.281 | 1.00 | 35.57 | B | O |
| ATOM | 2304 | N | ILE | 143 | 20.503 | −63.228 | 104.071 | 1.00 | 39.02 | B | N |
| ATOM | 2305 | CA | ILE | 143 | 21.385 | −62.575 | 105.028 | 1.00 | 42.74 | B | C |
| ATOM | 2306 | CB | ILE | 143 | 22.843 | −62.584 | 104.472 | 1.00 | 42.59 | B | C |
| ATOM | 2307 | CG2 | ILE | 143 | 23.827 | −62.089 | 105.529 | 1.00 | 42.50 | B | C |
| ATOM | 2308 | CG1 | ILE | 143 | 23.209 | −64.007 | 104.032 | 1.00 | 42.81 | B | C |
| ATOM | 2309 | CD1 | ILE | 143 | 24.580 | −64.142 | 103.438 | 1.00 | 43.32 | B | C |
| ATOM | 2310 | C | ILE | 143 | 20.970 | −61.147 | 105.388 | 1.00 | 45.22 | B | C |
| ATOM | 2311 | O | ILE | 143 | 20.209 | −60.940 | 106.333 | 1.00 | 46.12 | B | O |
| ATOM | 2312 | N | HIS | 144 | 21.462 | −60.165 | 104.636 | 1.00 | 48.10 | B | N |
| ATOM | 2313 | CA | HIS | 144 | 21.138 | −58.766 | 104.905 | 1.00 | 50.43 | B | C |
| ATOM | 2314 | CB | HIS | 144 | 22.347 | −58.077 | 105.549 | 1.00 | 51.71 | B | C |
| ATOM | 2315 | CG | HIS | 144 | 22.639 | −58.553 | 106.941 | 1.00 | 53.18 | B | C |
| ATOM | 2316 | CD2 | HIS | 144 | 21.840 | −59.129 | 107.873 | 1.00 | 53.68 | B | C |
| ATOM | 2317 | ND1 | HIS | 144 | 23.885 | −58.447 | 107.520 | 1.00 | 53.87 | B | N |
| ATOM | 2318 | CE1 | HIS | 144 | 23.843 | −58.939 | 108.747 | 1.00 | 53.89 | B | C |
| ATOM | 2319 | NE2 | HIS | 144 | 22.613 | −59.360 | 108.985 | 1.00 | 53.40 | B | N |
| ATOM | 2320 | C | HIS | 144 | 20.688 | −58.010 | 103.655 | 1.00 | 50.90 | B | C |
| ATOM | 2321 | O | HIS | 144 | 21.479 | −57.322 | 103.006 | 1.00 | 51.65 | B | O |
| ATOM | 2322 | N | HIS | 145 | 19.403 | −58.149 | 103.339 | 1.00 | 51.36 | B | N |
| ATOM | 2323 | CA | HIS | 145 | 18.778 | −57.503 | 102.183 | 1.00 | 51.48 | B | C |
| ATOM | 2324 | CB | HIS | 145 | 18.251 | −58.577 | 101.223 | 1.00 | 52.46 | B | C |
| ATOM | 2325 | CG | HIS | 145 | 18.066 | −58.105 | 99.812 | 1.00 | 53.73 | B | C |
| ATOM | 2326 | CD2 | HIS | 145 | 16.958 | −57.714 | 99.140 | 1.00 | 54.04 | B | C |
| ATOM | 2327 | ND1 | HIS | 145 | 19.109 | −58.015 | 98.915 | 1.00 | 54.94 | B | N |
| ATOM | 2328 | CE1 | HIS | 145 | 18.651 | −57.592 | 97.750 | 1.00 | 54.82 | B | C |
| ATOM | 2329 | NE2 | HIS | 145 | 17.348 | −57.401 | 97.859 | 1.00 | 54.54 | B | N |
| ATOM | 2330 | C | HIS | 145 | 17.610 | −56.629 | 102.673 | 1.00 | 50.58 | B | C |
| ATOM | 2331 | O | HIS | 145 | 16.980 | −56.929 | 103.691 | 1.00 | 50.62 | B | O |
| ATOM | 2332 | N | GLN | 146 | 17.325 | −55.552 | 101.950 | 1.00 | 49.68 | B | N |
| ATOM | 2333 | CA | GLN | 146 | 16.227 | −54.650 | 102.303 | 1.00 | 48.58 | B | C |
| ATOM | 2334 | CB | GLN | 146 | 16.264 | −53.426 | 101.373 | 1.00 | 50.29 | B | C |
| ATOM | 2335 | CG | GLN | 146 | 15.242 | −52.329 | 101.656 | 1.00 | 52.37 | B | C |
| ATOM | 2336 | CD | GLN | 146 | 14.090 | −52.316 | 100.661 | 1.00 | 53.97 | B | C |
| ATOM | 2337 | OE1 | GLN | 146 | 14.299 | −52.395 | 99.446 | 1.00 | 55.36 | B | O |
| ATOM | 2338 | NE2 | GLN | 146 | 12.867 | −52.216 | 101.172 | 1.00 | 53.39 | B | N |
| ATOM | 2339 | C | GLN | 146 | 14.902 | −55.412 | 102.152 | 1.00 | 46.43 | B | C |
| ATOM | 2340 | O | GLN | 146 | 14.772 | −56.250 | 101.259 | 1.00 | 47.01 | B | O |
| ATOM | 2341 | N | PRO | 147 | 13.906 | −55.140 | 103.024 | 1.00 | 43.87 | B | N |
| ATOM | 2342 | CD | PRO | 147 | 13.896 | −54.126 | 104.094 | 1.00 | 42.94 | B | C |
| ATOM | 2343 | CA | PRO | 147 | 12.604 | −55.823 | 102.952 | 1.00 | 40.85 | B | C |
| ATOM | 2344 | CB | PRO | 147 | 11.805 | −55.171 | 104.081 | 1.00 | 41.31 | B | C |
| ATOM | 2345 | CG | PRO | 147 | 12.427 | −53.812 | 104.211 | 1.00 | 42.30 | B | C |
| ATOM | 2346 | C | PRO | 147 | 11.911 | −55.697 | 101.594 | 1.00 | 37.44 | B | C |
| ATOM | 2347 | O | PRO | 147 | 12.368 | −54.956 | 100.733 | 1.00 | 36.62 | B | O |
| ATOM | 2348 | N | LEU | 148 | 10.812 | −56.428 | 101.408 | 1.00 | 34.45 | B | N |
| ATOM | 2349 | CA | LEU | 148 | 10.076 | −56.402 | 100.143 | 1.00 | 31.79 | B | C |
| ATOM | 2350 | CB | LEU | 148 | 8.912 | −57.397 | 100.162 | 1.00 | 30.82 | B | C |
| ATOM | 2351 | CG | LEU | 148 | 8.883 | −58.528 | 99.124 | 1.00 | 31.47 | B | C |
| ATOM | 2352 | CD1 | LEU | 148 | 7.437 | −58.918 | 98.878 | 1.00 | 30.58 | B | C |
| ATOM | 2353 | CD2 | LEU | 148 | 9.531 | −58.102 | 97.817 | 1.00 | 30.15 | B | C |
| ATOM | 2354 | C | LEU | 148 | 9.521 | −55.018 | 99.849 | 1.00 | 29.43 | B | C |
| ATOM | 2355 | O | LEU | 148 | 8.779 | −54.456 | 100.653 | 1.00 | 28.77 | B | O |
| ATOM | 2356 | N | PRO | 149 | 9.871 | −54.448 | 98.690 | 1.00 | 27.64 | B | N |
| ATOM | 2357 | CD | PRO | 149 | 10.846 | −54.900 | 97.686 | 1.00 | 27.10 | B | C |
| ATOM | 2358 | CA | PRO | 149 | 9.358 | −53.114 | 98.361 | 1.00 | 26.65 | B | C |
| ATOM | 2359 | CB | PRO | 149 | 9.986 | −52.829 | 96.998 | 1.00 | 26.71 | B | C |
| ATOM | 2360 | CG | PRO | 149 | 11.296 | −53.591 | 97.075 | 1.00 | 26.43 | B | C |
| ATOM | 2361 | C | PRO | 149 | 7.833 | −53.103 | 98.311 | 1.00 | 25.49 | B | C |
| ATOM | 2362 | O | PRO | 149 | 7.201 | −54.132 | 98.090 | 1.00 | 25.29 | B | O |
| ATOM | 2363 | N | THR | 150 | 7.248 | −51.932 | 98.516 | 1.00 | 24.33 | B | N |
| ATOM | 2364 | CA | THR | 150 | 5.799 | −51.782 | 98.494 | 1.00 | 23.46 | B | C |
| ATOM | 2365 | CB | THR | 150 | 5.417 | −50.287 | 98.643 | 1.00 | 23.36 | B | C |
| ATOM | 2366 | OG1 | THR | 150 | 5.760 | −49.841 | 99.964 | 1.00 | 21.71 | B | O |
| ATOM | 2367 | CG2 | THR | 150 | 3.934 | −50.077 | 98.395 | 1.00 | 22.73 | B | C |
| ATOM | 2368 | C | THR | 150 | 5.120 | −52.357 | 97.244 | 1.00 | 23.83 | B | C |
| ATOM | 2369 | O | THR | 150 | 4.111 | −53.056 | 97.346 | 1.00 | 24.37 | B | O |
| ATOM | 2370 | N | LEU | 151 | 5.671 | −52.075 | 96.071 | 1.00 | 23.39 | B | N |
| ATOM | 2371 | CA | LEU | 151 | 5.086 | −52.549 | 94.818 | 1.00 | 24.18 | B | C |
| ATOM | 2372 | CB | LEU | 151 | 5.174 | −51.435 | 93.764 | 1.00 | 23.98 | B | C |
| ATOM | 2373 | CG | LEU | 151 | 3.943 | −50.542 | 93.516 | 1.00 | 26.19 | B | C |
| ATOM | 2374 | CD1 | LEU | 151 | 3.125 | −50.352 | 94.783 | 1.00 | 25.29 | B | C |
| ATOM | 2375 | CD2 | LEU | 151 | 4.406 | −49.199 | 92.956 | 1.00 | 25.33 | B | C |
| ATOM | 2376 | C | LEU | 151 | 5.692 | −53.843 | 94.256 | 1.00 | 24.80 | B | C |
| ATOM | 2377 | O | LEU | 151 | 5.289 | −54.307 | 93.188 | 1.00 | 24.09 | B | O |
| ATOM | 2378 | N | ALA | 152 | 6.651 | −54.433 | 94.965 | 1.00 | 25.14 | B | N |
| ATOM | 2379 | CA | ALA | 152 | 7.269 | −55.666 | 94.485 | 1.00 | 25.42 | B | C |
| ATOM | 2380 | CB | ALA | 152 | 8.449 | −56.032 | 95.357 | 1.00 | 25.27 | B | C |
| ATOM | 2381 | C | ALA | 152 | 6.255 | −56.800 | 94.483 | 1.00 | 25.45 | B | C |
| ATOM | 2382 | O | ALA | 152 | 5.516 | −56.990 | 95.447 | 1.00 | 26.33 | B | O |
| ATOM | 2383 | N | PRO | 153 | 6.182 | −57.558 | 93.385 | 1.00 | 25.59 | B | N |
| ATOM | 2384 | CD | PRO | 153 | 6.844 | −57.411 | 92.078 | 1.00 | 26.03 | B | C |
| ATOM | 2385 | CA | PRO | 153 | 5.214 | −58.658 | 93.373 | 1.00 | 25.15 | B | C |
| ATOM | 2386 | CB | PRO | 153 | 5.337 | −59.222 | 91.953 | 1.00 | 25.93 | B | C |
| ATOM | 2387 | CG | PRO | 153 | 6.712 | −58.795 | 91.514 | 1.00 | 27.37 | B | C |
| ATOM | 2388 | C | PRO | 153 | 5.538 | −59.683 | 94.457 | 1.00 | 24.29 | B | C |
| ATOM | 2389 | O | PRO | 153 | 6.705 | −59.912 | 94.776 | 1.00 | 23.77 | B | O |
| ATOM | 2390 | N | VAL | 154 | 4.502 | −60.283 | 95.031 | 1.00 | 23.14 | B | N |
| ATOM | 2391 | CA | VAL | 154 | 4.689 | −61.269 | 96.090 | 1.00 | 23.20 | B | C |
| ATOM | 2392 | CB | VAL | 154 | 3.612 | −61.118 | 97.205 | 1.00 | 23.14 | B | C |
| ATOM | 2393 | CG1 | VAL | 154 | 3.638 | −59.705 | 97.761 | 1.00 | 24.25 | B | C |
| ATOM | 2394 | CG2 | VAL | 154 | 2.233 | −61.440 | 96.656 | 1.00 | 22.99 | B | C |
| ATOM | 2395 | C | VAL | 154 | 4.669 | −62.710 | 95.589 | 1.00 | 22.02 | B | C |
| ATOM | 2396 | O | VAL | 154 | 4.882 | −63.631 | 96.364 | 1.00 | 21.64 | B | O |
| ATOM | 2397 | N | LEU | 155 | 4.413 | −62.901 | 94.298 | 1.00 | 21.95 | B | N |
| ATOM | 2398 | CA | LEU | 155 | 4.374 | −64.244 | 93.711 | 1.00 | 21.77 | B | C |
| ATOM | 2399 | CB | LEU | 155 | 4.170 | −64.164 | 92.191 | 1.00 | 21.08 | B | C |
| ATOM | 2400 | CG | LEU | 155 | 4.166 | −65.490 | 91.410 | 1.00 | 22.05 | B | C |
| ATOM | 2401 | CD1 | LEU | 155 | 3.033 | −66.385 | 91.892 | 1.00 | 20.48 | B | C |
| ATOM | 2402 | CD2 | LEU | 155 | 4.017 | −65.204 | 89.905 | 1.00 | 22.31 | B | C |
| ATOM | 2403 | C | LEU | 155 | 5.622 | −65.083 | 94.030 | 1.00 | 20.95 | B | C |
| ATOM | 2404 | O | LEU | 155 | 5.496 | −66.258 | 94.373 | 1.00 | 21.26 | B | O |
| ATOM | 2405 | N | PRO | 156 | 6.838 | −64.505 | 93.908 | 1.00 | 20.25 | B | N |
| ATOM | 2406 | CD | PRO | 156 | 7.210 | −63.205 | 93.313 | 1.00 | 20.55 | B | C |
| ATOM | 2407 | CA | PRO | 156 | 8.040 | −65.294 | 94.218 | 1.00 | 20.02 | B | C |
| ATOM | 2408 | CB | PRO | 156 | 9.178 | −64.316 | 93.942 | 1.00 | 19.60 | B | C |
| ATOM | 2409 | CG | PRO | 156 | 8.627 | −63.462 | 92.821 | 1.00 | 19.34 | B | C |
| ATOM | 2410 | C | PRO | 156 | 8.043 | −65.793 | 95.675 | 1.00 | 20.44 | B | C |
| ATOM | 2411 | O | PRO | 156 | 8.401 | −66.943 | 95.948 | 1.00 | 18.86 | B | O |
| ATOM | 2412 | N | LEU | 157 | 7.646 | −64.922 | 96.603 | 1.00 | 18.95 | B | N |
| ATOM | 2413 | CA | LEU | 157 | 7.584 | −65.298 | 98.012 | 1.00 | 18.72 | B | C |
| ATOM | 2414 | CB | LEU | 157 | 7.243 | −64.086 | 98.886 | 1.00 | 18.06 | B | C |
| ATOM | 2415 | CG | LEU | 157 | 7.139 | −64.367 | 100.390 | 1.00 | 18.20 | B | C |
| ATOM | 2416 | CD1 | LEU | 157 | 8.459 | −64.931 | 100.910 | 1.00 | 18.30 | B | C |
| ATOM | 2417 | CD2 | LEU | 157 | 6.800 | −63.078 | 101.133 | 1.00 | 18.86 | B | C |
| ATOM | 2418 | C | LEU | 157 | 6.524 | −66.376 | 98.210 | 1.00 | 18.16 | B | C |
| ATOM | 2419 | O | LEU | 157 | 6.762 | −67.369 | 98.892 | 1.00 | 17.47 | B | O |
| ATOM | 2420 | N | VAL | 158 | 5.353 | −66.174 | 97.612 | 1.00 | 17.84 | B | N |
| ATOM | 2421 | CA | VAL | 158 | 4.254 | −67.132 | 97.713 | 1.00 | 18.75 | B | C |
| ATOM | 2422 | CB | VAL | 158 | 3.016 | −66.637 | 96.928 | 1.00 | 19.19 | B | C |
| ATOM | 2423 | CG1 | VAL | 158 | 1.971 | −67.746 | 96.840 | 1.00 | 18.53 | B | C |
| ATOM | 2424 | CG2 | VAL | 158 | 2.431 | −65.397 | 97.610 | 1.00 | 19.01 | B | C |
| ATOM | 2425 | C | VAL | 158 | 4.656 | −68.504 | 97.167 | 1.00 | 19.26 | B | C |
| ATOM | 2426 | O | VAL | 158 | 4.372 | −69.539 | 97.773 | 1.00 | 17.72 | B | O |
| ATOM | 2427 | N | THR | 159 | 5.306 | −68.504 | 96.009 | 1.00 | 19.18 | B | N |
| ATOM | 2428 | CA | THR | 159 | 5.749 | −69.742 | 95.382 | 1.00 | 20.12 | B | C |
| ATOM | 2429 | CB | THR | 159 | 6.359 | −69.464 | 94.005 | 1.00 | 20.73 | B | C |
| ATOM | 2430 | OG1 | THR | 159 | 5.429 | −68.696 | 93.233 | 1.00 | 21.98 | B | O |
| ATOM | 2431 | CG2 | THR | 159 | 6.670 | −70.780 | 93.277 | 1.00 | 20.98 | B | C |
| ATOM | 2432 | C | THR | 159 | 6.786 | −70.418 | 96.270 | 1.00 | 19.12 | B | C |
| ATOM | 2433 | O | THR | 159 | 6.757 | −71.631 | 96.447 | 1.00 | 19.81 | B | O |
| ATOM | 2434 | N | HIS | 160 | 7.695 | −69.624 | 96.827 | 1.00 | 18.60 | B | N |
| ATOM | 2435 | CA | HIS | 160 | 8.725 | −70.143 | 97.722 | 1.00 | 19.62 | B | C |
| ATOM | 2436 | CB | HIS | 160 | 9.628 | −69.004 | 98.201 | 1.00 | 19.12 | B | C |
| ATOM | 2437 | CG | HIS | 160 | 10.583 | −69.405 | 99.283 | 1.00 | 20.97 | B | C |
| ATOM | 2438 | CD2 | HIS | 160 | 10.739 | −68.946 | 100.548 | 1.00 | 19.99 | B | C |
| ATOM | 2439 | ND1 | HIS | 160 | 11.547 | −70.374 | 99.106 | 1.00 | 21.00 | B | N |
| ATOM | 2440 | CE1 | HIS | 160 | 12.258 | −70.492 | 100.214 | 1.00 | 20.06 | B | C |
| ATOM | 2441 | NE2 | HIS | 160 | 11.787 | −69.637 | 101.103 | 1.00 | 18.80 | B | N |
| ATOM | 2442 | C | HIS | 160 | 8.069 | −70.835 | 98.922 | 1.00 | 18.85 | B | C |
| ATOM | 2443 | O | HIS | 160 | 8.465 | −71.934 | 99.309 | 1.00 | 19.48 | B | O |
| ATOM | 2444 | N | PHE | 161 | 7.065 | −70.192 | 99.510 | 1.00 | 18.30 | B | N |
| ATOM | 2445 | CA | PHE | 161 | 6.362 | −70.783 | 100.644 | 1.00 | 17.81 | B | C |
| ATOM | 2446 | CB | PHE | 161 | 5.333 | −69.799 | 101.218 | 1.00 | 17.09 | B | C |
| ATOM | 2447 | CG | PHE | 161 | 5.924 | −68.773 | 102.167 | 1.00 | 18.77 | B | C |
| ATOM | 2448 | CD1 | PHE | 161 | 7.253 | −68.861 | 102.576 | 1.00 | 18.08 | B | C |
| ATOM | 2449 | CD2 | PHE | 161 | 5.137 | −67.743 | 102.675 | 1.00 | 18.45 | B | C |
| ATOM | 2450 | CE1 | PHE | 161 | 7.788 | −67.943 | 103.478 | 1.00 | 19.86 | B | C |
| ATOM | 2451 | CE2 | PHE | 161 | 5.658 | −66.818 | 103.577 | 1.00 | 19.78 | B | C |
| ATOM | 2452 | CZ | PHE | 161 | 6.992 | −66.918 | 103.981 | 1.00 | 20.06 | B | C |
| ATOM | 2453 | C | PHE | 161 | 5.675 | −72.081 | 100.227 | 1.00 | 17.47 | B | C |
| ATOM | 2454 | O | PHE | 161 | 5.721 | −73.075 | 100.953 | 1.00 | 16.02 | B | O |
| ATOM | 2455 | N | ALA | 162 | 5.039 | −72.082 | 99.058 | 1.00 | 16.64 | B | N |
| ATOM | 2456 | CA | ALA | 162 | 4.361 | −73.286 | 98.588 | 1.00 | 17.85 | B | C |
| ATOM | 2457 | CB | ALA | 162 | 3.712 | −73.034 | 97.224 | 1.00 | 17.30 | B | C |
| ATOM | 2458 | C | ALA | 162 | 5.352 | −74.447 | 98.487 | 1.00 | 18.28 | B | C |
| ATOM | 2459 | O | ALA | 162 | 5.055 | −75.563 | 98.899 | 1.00 | 17.59 | B | O |
| ATOM | 2460 | N | ASP | 163 | 6.530 | −74.166 | 97.935 | 1.00 | 18.81 | B | N |
| ATOM | 2461 | CA | ASP | 163 | 7.569 | −75.170 | 97.763 | 1.00 | 20.29 | B | C |
| ATOM | 2462 | CB | ASP | 163 | 8.650 | −74.624 | 96.834 | 1.00 | 21.76 | B | C |
| ATOM | 2463 | CG | ASP | 163 | 8.147 | −74.448 | 95.414 | 1.00 | 22.93 | B | C |
| ATOM | 2464 | OD1 | ASP | 163 | 8.816 | −73.763 | 94.621 | 1.00 | 25.09 | B | O |
| ATOM | 2465 | OD2 | ASP | 163 | 7.080 | −75.005 | 95.093 | 1.00 | 23.14 | B | O |
| ATOM | 2466 | C | ASP | 163 | 8.183 | −75.671 | 99.072 | 1.00 | 20.12 | B | C |
| ATOM | 2467 | O | ASP | 163 | 8.307 | −76.873 | 99.258 | 1.00 | 18.59 | B | O |
| ATOM | 2468 | N | ILE | 164 | 8.564 | −74.780 | 99.986 | 1.00 | 19.96 | B | N |
| ATOM | 2469 | CA | ILE | 164 | 9.117 | −75.286 | 101.240 | 1.00 | 20.35 | B | C |
| ATOM | 2470 | CB | ILE | 164 | 9.877 | −74.193 | 102.071 | 1.00 | 20.04 | B | C |
| ATOM | 2471 | CG2 | ILE | 164 | 11.003 | −73.596 | 101.224 | 1.00 | 19.12 | B | C |
| ATOM | 2472 | CG1 | ILE | 164 | 8.934 | −73.093 | 102.555 | 1.00 | 20.01 | B | C |
| ATOM | 2473 | CD1 | ILE | 164 | 9.619 | −72.108 | 103.497 | 1.00 | 18.11 | B | C |
| ATOM | 2474 | C | ILE | 164 | 8.014 | −75.950 | 102.076 | 1.00 | 20.60 | B | C |
| ATOM | 2475 | O | ILE | 164 | 8.287 | −76.890 | 102.819 | 1.00 | 20.54 | B | O |
| ATOM | 2476 | N | ASN | 165 | 6.766 | −75.496 | 101.939 | 1.00 | 19.91 | B | N |
| ATOM | 2477 | CA | ASN | 165 | 5.664 | −76.123 | 102.673 | 1.00 | 19.83 | B | C |
| ATOM | 2478 | CB | ASN | 165 | 4.341 | −75.370 | 102.464 | 1.00 | 18.46 | B | C |
| ATOM | 2479 | CG | ASN | 165 | 4.268 | −74.061 | 103.244 | 1.00 | 19.03 | B | C |
| ATOM | 2480 | OD1 | ASN | 165 | 5.070 | −73.812 | 104.145 | 1.00 | 17.31 | B | O |
| ATOM | 2481 | ND2 | ASN | 165 | 3.290 | −73.221 | 102.900 | 1.00 | 17.09 | B | N |
| ATOM | 2482 | C | ASN | 165 | 5.497 | −77.565 | 102.174 | 1.00 | 20.04 | B | C |
| ATOM | 2483 | O | ASN | 165 | 5.334 | −78.500 | 102.960 | 1.00 | 19.45 | B | O |
| ATOM | 2484 | N | THR | 166 | 5.525 | −77.741 | 100.859 | 1.00 | 20.25 | B | N |
| ATOM | 2485 | CA | THR | 166 | 5.379 | −79.071 | 100.279 | 1.00 | 20.86 | B | C |
| ATOM | 2486 | CB | THR | 166 | 5.359 | −78.981 | 98.741 | 1.00 | 21.41 | B | C |
| ATOM | 2487 | OG1 | THR | 166 | 4.211 | −78.224 | 98.341 | 1.00 | 22.00 | B | O |
| ATOM | 2488 | CG2 | THR | 166 | 5.293 | −80.368 | 98.104 | 1.00 | 20.43 | B | C |
| ATOM | 2489 | C | THR | 166 | 6.537 | −79.947 | 100.759 | 1.00 | 20.47 | B | C |
| ATOM | 2490 | O | THR | 166 | 6.338 | −81.079 | 101.198 | 1.00 | 19.94 | B | O |
| ATOM | 2491 | N | PHE | 167 | 7.745 | −79.403 | 100.682 | 1.00 | 19.96 | B | N |
| ATOM | 2492 | CA | PHE | 167 | 8.944 | −80.099 | 101.133 | 1.00 | 20.53 | B | C |
| ATOM | 2493 | CB | PHE | 167 | 10.153 | −79.166 | 100.994 | 1.00 | 20.58 | B | C |
| ATOM | 2494 | CG | PHE | 167 | 11.373 | −79.620 | 101.752 | 1.00 | 21.61 | B | C |
| ATOM | 2495 | CD1 | PHE | 167 | 12.098 | −80.739 | 101.338 | 1.00 | 21.76 | B | C |
| ATOM | 2496 | CD2 | PHE | 167 | 11.812 | −78.909 | 102.868 | 1.00 | 20.70 | B | C |
| ATOM | 2497 | CE1 | PHE | 167 | 13.254 | −81.145 | 102.025 | 1.00 | 22.38 | B | C |
| ATOM | 2498 | CE2 | PHE | 167 | 12.959 | −79.303 | 103.563 | 1.00 | 22.67 | B | C |
| ATOM | 2499 | CZ | PHE | 167 | 13.685 | −80.426 | 103.137 | 1.00 | 22.67 | B | C |
| ATOM | 2500 | C | PHE | 167 | 8.790 | −80.557 | 102.592 | 1.00 | 20.25 | B | C |
| ATOM | 2501 | O | PHE | 167 | 9.011 | −81.730 | 102.904 | 1.00 | 20.74 | B | O |
| ATOM | 2502 | N | MET | 168 | 8.403 | −79.640 | 103.479 | 1.00 | 18.80 | B | N |
| ATOM | 2503 | CA | MET | 168 | 8.235 | −79.971 | 104.897 | 1.00 | 18.84 | B | C |
| ATOM | 2504 | CB | MET | 168 | 7.858 | −78.722 | 105.712 | 1.00 | 17.54 | B | C |
| ATOM | 2505 | CG | MET | 168 | 9.004 | −77.727 | 105.904 | 1.00 | 16.54 | B | C |
| ATOM | 2506 | SD | MET | 168 | 8.609 | −76.401 | 107.082 | 1.00 | 16.62 | B | S |
| ATOM | 2507 | CE | MET | 168 | 7.812 | −75.178 | 106.019 | 1.00 | 15.56 | B | C |
| ATOM | 2508 | C | MET | 168 | 7.215 | −81.078 | 105.163 | 1.00 | 18.68 | B | C |
| ATOM | 2509 | O | MET | 168 | 7.461 | −81.969 | 105.970 | 1.00 | 17.64 | B | O |
| ATOM | 2510 | N | VAL | 169 | 6.065 | −81.024 | 104.503 | 1.00 | 19.45 | B | N |
| ATOM | 2511 | CA | VAL | 169 | 5.057 | −82.064 | 104.702 | 1.00 | 20.76 | B | C |
| ATOM | 2512 | CB | VAL | 169 | 3.770 | −81.768 | 103.889 | 1.00 | 21.33 | B | C |
| ATOM | 2513 | CG1 | VAL | 169 | 2.833 | −82.969 | 103.936 | 1.00 | 20.85 | B | C |
| ATOM | 2514 | CG2 | VAL | 169 | 3.072 | −80.531 | 104.458 | 1.00 | 21.08 | B | C |
| ATOM | 2515 | C | VAL | 169 | 5.642 | −83.414 | 104.275 | 1.00 | 21.26 | B | C |
| ATOM | 2516 | O | VAL | 169 | 5.475 | −84.420 | 104.965 | 1.00 | 20.54 | B | O |
| ATOM | 2517 | N | LEU | 170 | 6.337 | −83.435 | 103.139 | 1.00 | 21.18 | B | N |
| ATOM | 2518 | CA | LEU | 170 | 6.959 | −84.670 | 102.659 | 1.00 | 21.80 | B | C |
| ATOM | 2519 | CB | LEU | 170 | 7.695 | −84.421 | 101.338 | 1.00 | 22.86 | B | C |
| ATOM | 2520 | CG | LEU | 170 | 6.802 | −84.091 | 100.139 | 1.00 | 24.31 | B | C |
| ATOM | 2521 | CD1 | LEU | 170 | 7.655 | −83.710 | 98.941 | 1.00 | 24.24 | B | C |
| ATOM | 2522 | CD2 | LEU | 170 | 5.928 | −85.300 | 99.818 | 1.00 | 24.97 | B | C |
| ATOM | 2523 | C | LEU | 170 | 7.942 | −85.212 | 103.702 | 1.00 | 21.40 | B | C |
| ATOM | 2524 | O | LEU | 170 | 8.051 | −86.426 | 103.891 | 1.00 | 20.50 | B | O |
| ATOM | 2525 | N | GLN | 171 | 8.660 | −84.312 | 104.371 | 1.00 | 20.25 | B | N |
| ATOM | 2526 | CA | GLN | 171 | 9.609 | −84.721 | 105.402 | 1.00 | 20.37 | B | C |
| ATOM | 2527 | CB | GLN | 171 | 10.510 | −83.544 | 105.808 | 1.00 | 18.76 | B | C |
| ATOM | 2528 | CG | GLN | 171 | 11.490 | −83.112 | 104.719 | 1.00 | 17.74 | B | C |
| ATOM | 2529 | CD | GLN | 171 | 12.377 | −84.259 | 104.254 | 1.00 | 18.92 | B | C |
| ATOM | 2530 | OE1 | GLN | 171 | 13.124 | −84.841 | 105.034 | 1.00 | 20.91 | B | O |
| ATOM | 2531 | NE2 | GLN | 171 | 12.290 | −84.590 | 102.987 | 1.00 | 17.61 | B | N |
| ATOM | 2532 | C | GLN | 171 | 8.879 | −85.276 | 106.626 | 1.00 | 20.12 | B | C |
| ATOM | 2533 | O | GLN | 171 | 9.344 | −86.229 | 107.249 | 1.00 | 19.85 | B | O |
| ATOM | 2534 | N | VAL | 172 | 7.740 | −84.677 | 106.971 | 1.00 | 20.71 | B | N |
| ATOM | 2535 | CA | VAL | 172 | 6.944 | −85.137 | 108.112 | 1.00 | 21.73 | B | C |
| ATOM | 2536 | CB | VAL | 172 | 5.733 | −84.207 | 108.363 | 1.00 | 22.96 | B | C |
| ATOM | 2537 | CG1 | VAL | 172 | 4.789 | −84.833 | 109.384 | 1.00 | 22.67 | B | C |
| ATOM | 2538 | CG2 | VAL | 172 | 6.222 | −82.851 | 108.874 | 1.00 | 22.87 | B | C |
| ATOM | 2539 | C | VAL | 172 | 6.442 | −86.561 | 107.853 | 1.00 | 21.64 | B | C |
| ATOM | 2540 | O | VAL | 172 | 6.419 | −87.408 | 108.755 | 1.00 | 20.64 | B | O |
| ATOM | 2541 | N | ILE | 173 | 6.044 | −86.815 | 106.612 | 1.00 | 21.03 | B | N |
| ATOM | 2542 | CA | ILE | 173 | 5.579 | −88.132 | 106.219 | 1.00 | 21.75 | B | C |
| ATOM | 2543 | CB | ILE | 173 | 5.130 | −88.135 | 104.725 | 1.00 | 21.85 | B | C |
| ATOM | 2544 | CG2 | ILE | 173 | 4.791 | −89.563 | 104.271 | 1.00 | 22.44 | B | C |
| ATOM | 2545 | CG1 | ILE | 173 | 3.908 | −87.222 | 104.561 | 1.00 | 21.48 | B | C |
| ATOM | 2546 | CD1 | ILE | 173 | 3.360 | −87.129 | 103.143 | 1.00 | 20.80 | B | C |
| ATOM | 2547 | C | ILE | 173 | 6.725 | −89.126 | 106.449 | 1.00 | 21.99 | B | C |
| ATOM | 2548 | O | ILE | 173 | 6.512 | −90.212 | 106.986 | 1.00 | 20.98 | B | O |
| ATOM | 2549 | N | LYS | 174 | 7.942 | −88.742 | 106.057 | 1.00 | 22.27 | B | N |
| ATOM | 2550 | CA | LYS | 174 | 9.117 | −89.596 | 106.252 | 1.00 | 22.72 | B | C |
| ATOM | 2551 | CB | LYS | 174 | 10.365 | −88.925 | 105.671 | 1.00 | 23.87 | B | C |
| ATOM | 2552 | CG | LYS | 174 | 10.571 | −89.108 | 104.171 | 1.00 | 27.23 | B | C |
| ATOM | 2553 | CD | LYS | 174 | 11.763 | −88.274 | 103.704 | 1.00 | 29.56 | B | C |
| ATOM | 2554 | CE | LYS | 174 | 12.282 | −88.701 | 102.342 | 1.00 | 32.40 | B | C |
| ATOM | 2555 | NZ | LYS | 174 | 13.087 | −89.966 | 102.435 | 1.00 | 34.29 | B | N |
| ATOM | 2556 | C | LYS | 174 | 9.344 | −89.888 | 107.743 | 1.00 | 21.87 | B | C |
| ATOM | 2557 | O | LYS | 174 | 9.709 | −91.007 | 108.133 | 1.00 | 20.46 | B | O |
| ATOM | 2558 | N | PHE | 175 | 9.141 | −88.862 | 108.563 | 1.00 | 21.53 | B | N |
| ATOM | 2559 | CA | PHE | 175 | 9.291 | −88.966 | 110.008 | 1.00 | 21.67 | B | C |
| ATOM | 2560 | CB | PHE | 175 | 9.027 | −87.599 | 110.643 | 1.00 | 21.39 | B | C |
| ATOM | 2561 | CG | PHE | 175 | 8.820 | −87.642 | 112.134 | 1.00 | 22.55 | B | C |
| ATOM | 2562 | CD1 | PHE | 175 | 9.878 | −87.933 | 112.995 | 1.00 | 21.38 | B | C |
| ATOM | 2563 | CD2 | PHE | 175 | 7.563 | −87.373 | 112.679 | 1.00 | 22.64 | B | C |
| ATOM | 2564 | CE1 | PHE | 175 | 9.690 | −87.953 | 114.374 | 1.00 | 21.89 | B | C |
| ATOM | 2565 | CE2 | PHE | 175 | 7.366 | −87.393 | 114.061 | 1.00 | 23.00 | B | C |
| ATOM | 2566 | CZ | PHE | 175 | 8.435 | −87.684 | 114.910 | 1.00 | 22.35 | B | C |
| ATOM | 2567 | C | PHE | 175 | 8.324 | −89.999 | 110.581 | 1.00 | 21.68 | B | C |
| ATOM | 2568 | O | PHE | 175 | 8.718 | −90.866 | 111.362 | 1.00 | 21.47 | B | O |
| ATOM | 2569 | N | THR | 176 | 7.058 | −89.910 | 110.188 | 1.00 | 22.73 | B | N |
| ATOM | 2570 | CA | THR | 176 | 6.050 | −90.838 | 110.686 | 1.00 | 24.75 | B | C |
| ATOM | 2571 | CB | THR | 176 | 4.616 | −90.434 | 110.230 | 1.00 | 25.26 | B | C |
| ATOM | 2572 | OG1 | THR | 176 | 4.482 | −90.632 | 108.820 | 1.00 | 24.98 | B | O |
| ATOM | 2573 | CG2 | THR | 176 | 4.331 | −88.961 | 110.565 | 1.00 | 24.45 | B | C |
| ATOM | 2574 | C | THR | 176 | 6.318 | −92.285 | 110.257 | 1.00 | 25.69 | B | C |
| ATOM | 2575 | O | THR | 176 | 6.172 | −93.206 | 111.065 | 1.00 | 24.44 | B | O |
| ATOM | 2576 | N | LYS | 177 | 6.737 | −92.480 | 109.005 | 1.00 | 26.56 | B | N |
| ATOM | 2577 | CA | LYS | 177 | 7.007 | −93.824 | 108.489 | 1.00 | 28.90 | B | C |
| ATOM | 2578 | CB | LYS | 177 | 7.051 | −93.805 | 106.958 | 1.00 | 29.06 | B | C |
| ATOM | 2579 | CG | LYS | 177 | 5.721 | −93.404 | 106.327 | 1.00 | 31.35 | B | C |
| ATOM | 2580 | CD | LYS | 177 | 5.742 | −93.550 | 104.815 | 1.00 | 33.93 | B | C |
| ATOM | 2581 | CE | LYS | 177 | 4.452 | −93.026 | 104.191 | 1.00 | 35.45 | B | C |
| ATOM | 2582 | NZ | LYS | 177 | 3.233 | −93.686 | 104.749 | 1.00 | 36.90 | B | N |
| ATOM | 2583 | C | LYS | 177 | 8.265 | −94.500 | 109.047 | 1.00 | 29.97 | B | C |
| ATOM | 2584 | O | LYS | 177 | 8.581 | −95.632 | 108.681 | 1.00 | 30.08 | B | O |
| ATOM | 2585 | N | ASP | 178 | 8.979 | −93.799 | 109.924 | 1.00 | 30.95 | B | N |
| ATOM | 2586 | CA | ASP | 178 | 10.171 | −94.338 | 110.580 | 1.00 | 31.86 | B | C |
| ATOM | 2587 | CB | ASP | 178 | 11.249 | −93.255 | 110.695 | 1.00 | 33.44 | B | C |
| ATOM | 2588 | CG | ASP | 178 | 12.365 | −93.427 | 109.689 | 1.00 | 35.31 | B | C |
| ATOM | 2589 | OD1 | ASP | 178 | 12.105 | −93.955 | 108.587 | 1.00 | 36.42 | B | O |
| ATOM | 2590 | OD2 | ASP | 178 | 13.506 | −93.018 | 109.994 | 1.00 | 37.36 | B | O |
| ATOM | 2591 | C | ASP | 178 | 9.770 | −94.814 | 111.986 | 1.00 | 31.51 | B | C |
| ATOM | 2592 | O | ASP | 178 | 10.600 | −95.282 | 112.760 | 1.00 | 31.62 | B | O |
| ATOM | 2593 | N | LEU | 179 | 8.485 | −94.689 | 112.299 | 1.00 | 31.35 | B | N |
| ATOM | 2594 | CA | LEU | 179 | 7.945 | −95.083 | 113.593 | 1.00 | 31.19 | B | C |
| ATOM | 2595 | CB | LEU | 179 | 7.050 | −93.964 | 114.136 | 1.00 | 31.02 | B | C |
| ATOM | 2596 | CG | LEU | 179 | 7.653 | −92.737 | 114.840 | 1.00 | 31.99 | B | C |
| ATOM | 2597 | CD1 | LEU | 179 | 8.991 | −92.337 | 114.255 | 1.00 | 31.68 | B | C |
| ATOM | 2598 | CD2 | LEU | 179 | 6.650 | −91.592 | 114.747 | 1.00 | 31.31 | B | C |
| ATOM | 2599 | C | LEU | 179 | 7.127 | −96.358 | 113.425 | 1.00 | 31.91 | B | C |
| ATOM | 2600 | O | LEU | 179 | 6.002 | −96.323 | 112.923 | 1.00 | 30.72 | B | O |
| ATOM | 2601 | N | PRO | 180 | 7.682 | −97.508 | 113.844 | 1.00 | 32.73 | B | N |
| ATOM | 2602 | CD | PRO | 180 | 8.944 | −97.731 | 114.565 | 1.00 | 32.93 | B | C |
| ATOM | 2603 | CA | PRO | 180 | 6.932 | −98.758 | 113.700 | 1.00 | 33.57 | B | C |
| ATOM | 2604 | CB | PRO | 180 | 7.842 | −99.789 | 114.374 | 1.00 | 33.58 | B | C |
| ATOM | 2605 | CG | PRO | 180 | 8.636 | −98.981 | 115.341 | 1.00 | 33.83 | B | C |
| ATOM | 2606 | C | PRO | 180 | 5.535 | −98.679 | 114.319 | 1.00 | 34.14 | B | C |
| ATOM | 2607 | O | PRO | 180 | 4.569 | −99.203 | 113.763 | 1.00 | 33.78 | B | O |
| ATOM | 2608 | N | VAL | 181 | 5.427 | −98.002 | 115.457 | 1.00 | 34.64 | B | N |
| ATOM | 2609 | CA | VAL | 181 | 4.140 | −97.857 | 116.119 | 1.00 | 35.27 | B | C |
| ATOM | 2610 | CB | VAL | 181 | 4.265 | −97.083 | 117.441 | 1.00 | 35.75 | B | C |
| ATOM | 2611 | CG1 | VAL | 181 | 2.911 | −96.993 | 118.104 | 1.00 | 36.59 | B | C |
| ATOM | 2612 | CG2 | VAL | 181 | 5.258 | −97.782 | 118.362 | 1.00 | 36.26 | B | C |
| ATOM | 2613 | C | VAL | 181 | 3.148 | −97.131 | 115.220 | 1.00 | 35.36 | B | C |
| ATOM | 2614 | O | VAL | 181 | 1.986 | −97.515 | 115.151 | 1.00 | 35.38 | B | O |
| ATOM | 2615 | N | PHE | 182 | 3.599 | −96.079 | 114.538 | 1.00 | 35.49 | B | N |
| ATOM | 2616 | CA | PHE | 182 | 2.726 | −95.330 | 113.630 | 1.00 | 35.30 | B | C |
| ATOM | 2617 | CB | PHE | 182 | 3.431 | −94.071 | 113.117 | 1.00 | 33.92 | B | C |
| ATOM | 2618 | CG | PHE | 182 | 2.597 | −93.254 | 112.164 | 1.00 | 32.96 | B | C |
| ATOM | 2619 | CD1 | PHE | 182 | 1.647 | −92.359 | 112.640 | 1.00 | 32.81 | B | C |
| ATOM | 2620 | CD2 | PHE | 182 | 2.760 | −93.386 | 110.786 | 1.00 | 32.65 | B | C |
| ATOM | 2621 | CE1 | PHE | 182 | 0.871 | −91.604 | 111.759 | 1.00 | 32.61 | B | C |
| ATOM | 2622 | CE2 | PHE | 182 | 1.993 | −92.640 | 109.897 | 1.00 | 32.30 | B | C |
| ATOM | 2623 | CZ | PHE | 182 | 1.047 | −91.746 | 110.382 | 1.00 | 32.70 | B | C |
| ATOM | 2624 | C | PHE | 182 | 2.349 | −96.212 | 112.439 | 1.00 | 36.18 | B | C |
| ATOM | 2625 | O | PHE | 182 | 1.212 | −96.183 | 111.966 | 1.00 | 35.72 | B | O |
| ATOM | 2626 | N | ARG | 183 | 3.315 | −96.990 | 111.958 | 1.00 | 37.31 | B | N |
| ATOM | 2627 | CA | ARG | 183 | 3.098 | −97.885 | 110.830 | 1.00 | 39.17 | B | C |
| ATOM | 2628 | CB | ARG | 183 | 4.427 | −98.506 | 110.383 | 1.00 | 39.31 | B | C |
| ATOM | 2629 | CG | ARG | 183 | 5.192 | −97.668 | 109.381 | 1.00 | 39.52 | B | C |
| ATOM | 2630 | CD | ARG | 183 | 6.380 | −98.428 | 108.809 | 1.00 | 41.16 | B | C |
| ATOM | 2631 | NE | ARG | 183 | 7.596 | −98.227 | 109.593 | 1.00 | 43.06 | B | N |
| ATOM | 2632 | CZ | ARG | 183 | 8.263 | −99.187 | 110.224 | 1.00 | 43.53 | B | C |
| ATOM | 2633 | NH1 | ARG | 183 | 7.840 | −100.445 | 110.179 | 1.00 | 43.79 | B | N |
| ATOM | 2634 | NH2 | ARG | 183 | 9.370 | −98.885 | 110.892 | 1.00 | 43.85 | B | N |
| ATOM | 2635 | C | ARG | 183 | 2.094 | −99.003 | 111.108 | 1.00 | 39.98 | B | C |
| ATOM | 2636 | O | ARG | 183 | 1.442 | −99.493 | 110.187 | 1.00 | 39.52 | B | O |
| ATOM | 2637 | N | SER | 184 | 1.962 | −99.398 | 112.372 | 1.00 | 41.05 | B | N |
| ATOM | 2638 | CA | SER | 184 | 1.054 | −100.481 | 112.735 | 1.00 | 42.09 | B | C |
| ATOM | 2639 | CB | SER | 184 | 1.462 | −101.079 | 114.083 | 1.00 | 42.35 | B | C |
| ATOM | 2640 | OG | SER | 184 | 1.270 | −100.153 | 115.138 | 1.00 | 44.21 | B | O |
| ATOM | 2641 | C | SER | 184 | −0.434 | −100.137 | 112.765 | 1.00 | 42.40 | B | C |
| ATOM | 2642 | O | SER | 184 | −1.263 | −101.034 | 112.899 | 1.00 | 42.68 | B | O |
| ATOM | 2643 | N | LEU | 185 | −0.788 | −98.860 | 112.648 | 1.00 | 42.68 | B | N |
| ATOM | 2644 | CA | LEU | 185 | −2.201 | −98.499 | 112.660 | 1.00 | 42.79 | B | C |
| ATOM | 2645 | CB | LEU | 185 | −2.441 | −97.131 | 113.330 | 1.00 | 43.53 | B | C |
| ATOM | 2646 | CG | LEU | 185 | −1.421 | −96.007 | 113.534 | 1.00 | 44.02 | B | C |
| ATOM | 2647 | CD1 | LEU | 185 | −2.140 | −94.742 | 113.988 | 1.00 | 43.73 | B | C |
| ATOM | 2648 | CD2 | LEU | 185 | −0.417 | −96.404 | 114.589 | 1.00 | 44.56 | B | C |
| ATOM | 2649 | C | LEU | 185 | −2.831 | −98.510 | 111.271 | 1.00 | 42.68 | B | C |
| ATOM | 2650 | O | LEU | 185 | −2.133 | −98.457 | 110.258 | 1.00 | 42.19 | B | O |
| ATOM | 2651 | N | PRO | 186 | −4.171 | −98.598 | 111.209 | 1.00 | 42.84 | B | N |
| ATOM | 2652 | CD | PRO | 186 | −5.129 | −98.602 | 112.327 | 1.00 | 42.91 | B | C |
| ATOM | 2653 | CA | PRO | 186 | −4.877 | −98.615 | 109.926 | 1.00 | 43.29 | B | C |
| ATOM | 2654 | CB | PRO | 186 | −6.351 | −98.617 | 110.337 | 1.00 | 42.26 | B | C |
| ATOM | 2655 | CG | PRO | 186 | −6.338 | −97.972 | 111.696 | 1.00 | 43.70 | B | C |
| ATOM | 2656 | C | PRO | 186 | −4.512 | −97.421 | 109.054 | 1.00 | 43.49 | B | C |
| ATOM | 2657 | O | PRO | 186 | −4.262 | −96.326 | 109.556 | 1.00 | 43.14 | B | O |
| ATOM | 2658 | N | ILE | 187 | −4.481 | −97.650 | 107.747 | 1.00 | 43.61 | B | N |
| ATOM | 2659 | CA | ILE | 187 | −4.137 | −96.613 | 106.784 | 1.00 | 43.56 | B | C |
| ATOM | 2660 | CB | ILE | 187 | −4.406 | −97.082 | 105.337 | 1.00 | 43.82 | B | C |
| ATOM | 2661 | CG2 | ILE | 187 | −3.939 | −96.018 | 104.353 | 1.00 | 43.79 | B | C |
| ATOM | 2662 | CG1 | ILE | 187 | −3.667 | −98.395 | 105.063 | 1.00 | 44.06 | B | C |
| ATOM | 2663 | CD1 | ILE | 187 | −2.156 | −98.288 | 105.188 | 1.00 | 44.66 | B | C |
| ATOM | 2664 | C | ILE | 187 | −4.916 | −95.325 | 107.021 | 1.00 | 43.43 | B | C |
| ATOM | 2665 | O | ILE | 187 | −4.350 | −94.232 | 106.975 | 1.00 | 42.67 | B | O |
| ATOM | 2666 | N | GLU | 188 | −6.214 | −95.451 | 107.272 | 1.00 | 43.55 | B | N |
| ATOM | 2667 | CA | GLU | 188 | −7.039 | −94.273 | 107.496 | 1.00 | 44.32 | B | C |
| ATOM | 2668 | CB | GLU | 188 | −8.514 | −94.658 | 107.582 | 1.00 | 45.62 | B | C |
| ATOM | 2669 | CG | GLU | 188 | −9.421 | −93.446 | 107.496 | 1.00 | 48.84 | B | C |
| ATOM | 2670 | CD | GLU | 188 | −8.978 | −92.471 | 106.406 | 1.00 | 50.12 | B | C |
| ATOM | 2671 | OE1 | GLU | 188 | −8.919 | −92.875 | 105.223 | 1.00 | 51.29 | B | O |
| ATOM | 2672 | OE2 | GLU | 188 | −8.684 | −91.302 | 106.737 | 1.00 | 50.87 | B | O |
| ATOM | 2673 | C | GLU | 188 | −6.635 | −93.469 | 108.735 | 1.00 | 43.37 | B | C |
| ATOM | 2674 | O | GLU | 188 | −6.760 | −92.245 | 108.748 | 1.00 | 43.27 | B | O |
| ATOM | 2675 | N | ASP | 189 | −6.166 | −94.149 | 109.776 | 1.00 | 42.33 | B | N |
| ATOM | 2676 | CA | ASP | 189 | −5.720 | −93.457 | 110.979 | 1.00 | 41.76 | B | C |
| ATOM | 2677 | CB | ASP | 189 | −5.510 | −94.442 | 112.134 | 1.00 | 42.89 | B | C |
| ATOM | 2678 | CG | ASP | 189 | −6.811 | −94.832 | 112.806 | 1.00 | 44.75 | B | C |
| ATOM | 2679 | OD1 | ASP | 189 | −6.768 | −95.471 | 113.879 | 1.00 | 46.20 | B | O |
| ATOM | 2680 | OD2 | ASP | 189 | −7.882 | −94.498 | 112.258 | 1.00 | 45.42 | B | O |
| ATOM | 2681 | C | ASP | 189 | −4.417 | −92.718 | 110.683 | 1.00 | 40.38 | B | C |
| ATOM | 2682 | O | ASP | 189 | −4.237 | −91.578 | 111.102 | 1.00 | 40.12 | B | O |
| ATOM | 2683 | N | GLN | 190 | −3.515 | −93.369 | 109.955 | 1.00 | 39.17 | B | N |
| ATOM | 2684 | CA | GLN | 190 | −2.241 | −92.757 | 109.592 | 1.00 | 38.44 | B | C |
| ATOM | 2685 | CB | GLN | 190 | −1.416 | −93.702 | 108.725 | 1.00 | 37.85 | B | C |
| ATOM | 2686 | CG | GLN | 190 | −1.088 | −95.032 | 109.373 | 1.00 | 38.72 | B | C |
| ATOM | 2687 | CD | GLN | 190 | −0.103 | −95.854 | 108.560 | 1.00 | 38.85 | B | C |
| ATOM | 2688 | OE1 | GLN | 190 | 0.060 | −97.054 | 108.789 | 1.00 | 38.95 | B | O |
| ATOM | 2689 | NE2 | GLN | 190 | 0.567 | −95.209 | 107.611 | 1.00 | 39.18 | B | N |
| ATOM | 2690 | C | GLN | 190 | −2.486 | −91.468 | 108.822 | 1.00 | 37.77 | B | C |
| ATOM | 2691 | O | GLN | 190 | −1.846 | −90.451 | 109.077 | 1.00 | 38.02 | B | O |
| ATOM | 2692 | N | ILE | 191 | −3.418 | −91.521 | 107.877 | 1.00 | 37.50 | B | N |
| ATOM | 2693 | CA | ILE | 191 | −3.761 | −90.364 | 107.061 | 1.00 | 37.36 | B | C |
| ATOM | 2694 | CB | ILE | 191 | −4.787 | −90.737 | 105.964 | 1.00 | 38.55 | B | C |
| ATOM | 2695 | CG2 | ILE | 191 | −5.031 | −89.548 | 105.059 | 1.00 | 38.60 | B | C |
| ATOM | 2696 | CG1 | ILE | 191 | −4.278 | −91.921 | 105.138 | 1.00 | 39.51 | B | C |
| ATOM | 2697 | CD1 | ILE | 191 | −3.011 | −91.637 | 104.353 | 1.00 | 41.55 | B | C |
| ATOM | 2698 | C | ILE | 191 | −4.356 | −89.252 | 107.919 | 1.00 | 36.51 | B | C |
| ATOM | 2699 | O | ILE | 191 | −3.955 | −88.094 | 107.804 | 1.00 | 36.70 | B | O |
| ATOM | 2700 | N | SER | 192 | −5.312 | −89.606 | 108.776 | 1.00 | 35.03 | B | N |
| ATOM | 2701 | CA | SER | 192 | −5.959 | −88.628 | 109.646 | 1.00 | 34.26 | B | C |
| ATOM | 2702 | CB | SER | 192 | −7.086 | −89.286 | 110.446 | 1.00 | 34.74 | B | C |
| ATOM | 2703 | OG | SER | 192 | −8.107 | −89.757 | 109.586 | 1.00 | 35.76 | B | O |
| ATOM | 2704 | C | SER | 192 | −4.982 | −87.956 | 110.608 | 1.00 | 32.87 | B | C |
| ATOM | 2705 | O | SER | 192 | −5.078 | −86.753 | 110.853 | 1.00 | 31.92 | B | O |
| ATOM | 2706 | N | LEU | 193 | −4.054 | −88.732 | 111.160 | 1.00 | 31.50 | B | N |
| ATOM | 2707 | CA | LEU | 193 | −3.072 | −88.183 | 112.085 | 1.00 | 30.70 | B | C |
| ATOM | 2708 | CB | LEU | 193 | −2.295 | −89.305 | 112.786 | 1.00 | 29.93 | B | C |
| ATOM | 2709 | CG | LEU | 193 | −3.072 | −90.217 | 113.746 | 1.00 | 29.73 | B | C |
| ATOM | 2710 | CD1 | LEU | 193 | −2.099 | −91.157 | 114.450 | 1.00 | 29.19 | B | C |
| ATOM | 2711 | CD2 | LEU | 193 | −3.831 | −89.379 | 114.765 | 1.00 | 27.88 | B | C |
| ATOM | 2712 | C | LEU | 193 | −2.107 | −87.268 | 111.337 | 1.00 | 30.76 | B | C |
| ATOM | 2713 | O | LEU | 193 | −1.809 | −86.164 | 111.789 | 1.00 | 29.84 | B | O |
| ATOM | 2714 | N | LEU | 194 | −1.625 | −87.725 | 110.187 | 1.00 | 31.33 | B | N |
| ATOM | 2715 | CA | LEU | 194 | −0.706 | −86.924 | 109.389 | 1.00 | 32.09 | B | C |
| ATOM | 2716 | CB | LEU | 194 | −0.297 | −87.711 | 108.142 | 1.00 | 32.64 | B | C |
| ATOM | 2717 | CG | LEU | 194 | 0.922 | −87.259 | 107.333 | 1.00 | 34.23 | B | C |
| ATOM | 2718 | CD1 | LEU | 194 | 0.579 | −85.995 | 106.557 | 1.00 | 35.50 | B | C |
| ATOM | 2719 | CD2 | LEU | 194 | 2.116 | −87.032 | 108.260 | 1.00 | 33.53 | B | C |
| ATOM | 2720 | C | LEU | 194 | −1.370 | −85.589 | 109.017 | 1.00 | 32.06 | B | C |
| ATOM | 2721 | O | LEU | 194 | −0.831 | −84.523 | 109.303 | 1.00 | 31.90 | B | O |
| ATOM | 2722 | N | LYS | 195 | −2.549 | −85.651 | 108.402 | 1.00 | 32.21 | B | N |
| ATOM | 2723 | CA | LYS | 195 | −3.289 | −84.448 | 108.014 | 1.00 | 32.26 | B | C |
| ATOM | 2724 | CB | LYS | 195 | −4.643 | −84.832 | 107.404 | 1.00 | 34.49 | B | C |
| ATOM | 2725 | CG | LYS | 195 | −4.562 | −85.427 | 106.005 | 1.00 | 36.99 | B | C |
| ATOM | 2726 | CD | LYS | 195 | −5.851 | −86.146 | 105.619 | 1.00 | 38.55 | B | C |
| ATOM | 2727 | CE | LYS | 195 | −7.061 | −85.223 | 105.632 | 1.00 | 39.77 | B | C |
| ATOM | 2728 | NZ | LYS | 195 | −8.275 | −85.939 | 105.131 | 1.00 | 41.50 | B | N |
| ATOM | 2729 | C | LYS | 195 | −3.541 | −83.517 | 109.197 | 1.00 | 31.45 | B | C |
| ATOM | 2730 | O | LYS | 195 | −3.455 | −82.292 | 109.079 | 1.00 | 30.78 | B | O |
| ATOM | 2731 | N | GLY | 196 | −3.862 | −84.105 | 110.339 | 1.00 | 29.85 | B | N |
| ATOM | 2732 | CA | GLY | 196 | −4.141 | −83.303 | 111.509 | 1.00 | 28.37 | B | C |
| ATOM | 2733 | C | GLY | 196 | −2.946 | −82.676 | 112.195 | 1.00 | 26.84 | B | C |
| ATOM | 2734 | O | GLY | 196 | −3.090 | −81.621 | 112.806 | 1.00 | 26.88 | B | O |
| ATOM | 2735 | N | ALA | 197 | −1.768 | −83.284 | 112.085 | 1.00 | 25.28 | B | N |
| ATOM | 2736 | CA | ALA | 197 | −0.596 | −82.751 | 112.785 | 1.00 | 23.92 | B | C |
| ATOM | 2737 | CB | ALA | 197 | −0.125 | −83.776 | 113.816 | 1.00 | 23.40 | B | C |
| ATOM | 2738 | C | ALA | 197 | 0.608 | −82.269 | 111.978 | 1.00 | 22.01 | B | C |
| ATOM | 2739 | O | ALA | 197 | 1.536 | −81.706 | 112.550 | 1.00 | 20.56 | B | O |
| ATOM | 2740 | N | ALA | 198 | 0.606 | −82.483 | 110.668 | 1.00 | 20.97 | B | N |
| ATOM | 2741 | CA | ALA | 198 | 1.737 | −82.073 | 109.836 | 1.00 | 20.46 | B | C |
| ATOM | 2742 | CB | ALA | 198 | 1.406 | −82.284 | 108.349 | 1.00 | 20.75 | B | C |
| ATOM | 2743 | C | ALA | 198 | 2.188 | −80.631 | 110.071 | 1.00 | 19.62 | B | C |
| ATOM | 2744 | O | ALA | 198 | 3.359 | −80.381 | 110.365 | 1.00 | 19.14 | B | O |
| ATOM | 2745 | N | VAL | 199 | 1.262 | −79.689 | 109.935 | 1.00 | 19.02 | B | N |
| ATOM | 2746 | CA | VAL | 199 | 1.566 | −78.276 | 110.125 | 1.00 | 19.01 | B | C |
| ATOM | 2747 | CB | VAL | 199 | 0.365 | −77.401 | 109.738 | 1.00 | 19.49 | B | C |
| ATOM | 2748 | CG1 | VAL | 199 | 0.658 | −75.938 | 110.073 | 1.00 | 18.87 | B | C |
| ATOM | 2749 | CG2 | VAL | 199 | 0.074 | −77.563 | 108.243 | 1.00 | 19.44 | B | C |
| ATOM | 2750 | C | VAL | 199 | 1.986 | −77.946 | 111.555 | 1.00 | 18.82 | B | C |
| ATOM | 2751 | O | VAL | 199 | 2.856 | −77.104 | 111.766 | 1.00 | 19.08 | B | O |
| ATOM | 2752 | N | GLU | 200 | 1.369 | −78.605 | 112.531 | 1.00 | 17.97 | B | N |
| ATOM | 2753 | CA | GLU | 200 | 1.713 | −78.389 | 113.934 | 1.00 | 18.43 | B | C |
| ATOM | 2754 | CB | GLU | 200 | 0.751 | −79.152 | 114.850 | 1.00 | 18.15 | B | C |
| ATOM | 2755 | CG | GLU | 200 | −0.600 | −78.479 | 115.030 | 1.00 | 20.07 | B | C |
| ATOM | 2756 | CD | GLU | 200 | −1.570 | −79.307 | 115.866 | 1.00 | 21.56 | B | C |
| ATOM | 2757 | OE1 | GLU | 200 | −1.123 | −80.050 | 116.765 | 1.00 | 21.43 | B | O |
| ATOM | 2758 | OE2 | GLU | 200 | −2.785 | −79.197 | 115.630 | 1.00 | 22.35 | B | O |
| ATOM | 2759 | C | GLU | 200 | 3.138 | −78.854 | 114.202 | 1.00 | 17.94 | B | C |
| ATOM | 2760 | O | GLU | 200 | 3.920 | −78.148 | 114.830 | 1.00 | 18.17 | B | O |
| ATOM | 2761 | N | ILE | 201 | 3.469 | −80.047 | 113.716 | 1.00 | 17.97 | B | N |
| ATOM | 2762 | CA | ILE | 201 | 4.799 | −80.612 | 113.897 | 1.00 | 17.91 | B | C |
| ATOM | 2763 | CB | ILE | 201 | 4.840 | −82.051 | 113.335 | 1.00 | 18.26 | B | C |
| ATOM | 2764 | CG2 | ILE | 201 | 6.266 | −82.572 | 113.260 | 1.00 | 17.92 | B | C |
| ATOM | 2765 | CG1 | ILE | 201 | 3.986 | −82.953 | 114.233 | 1.00 | 17.28 | B | C |
| ATOM | 2766 | CD1 | ILE | 201 | 3.815 | −84.368 | 113.701 | 1.00 | 18.61 | B | C |
| ATOM | 2767 | C | ILE | 201 | 5.869 | −79.731 | 113.255 | 1.00 | 17.32 | B | C |
| ATOM | 2768 | O | ILE | 201 | 6.953 | −79.547 | 113.815 | 1.00 | 18.72 | B | O |
| ATOM | 2769 | N | CYS | 202 | 5.567 | −79.165 | 112.095 | 1.00 | 16.54 | B | N |
| ATOM | 2770 | CA | CYS | 202 | 6.522 | −78.282 | 111.434 | 1.00 | 16.29 | B | C |
| ATOM | 2771 | CB | CYS | 202 | 5.996 | −77.845 | 110.062 | 1.00 | 16.39 | B | C |
| ATOM | 2772 | SG | CYS | 202 | 5.924 | −79.183 | 108.838 | 1.00 | 16.45 | B | S |
| ATOM | 2773 | C | CYS | 202 | 6.822 | −77.049 | 112.295 | 1.00 | 15.32 | B | C |
| ATOM | 2774 | O | CYS | 202 | 7.968 | −76.624 | 112.386 | 1.00 | 14.15 | B | O |
| ATOM | 2775 | N | HIS | 203 | 5.806 | −76.463 | 112.924 | 1.00 | 15.37 | B | N |
| ATOM | 2776 | CA | HIS | 203 | 6.061 | −75.290 | 113.768 | 1.00 | 15.80 | B | C |
| ATOM | 2777 | CB | HIS | 203 | 4.750 | −74.610 | 114.164 | 1.00 | 15.84 | B | C |
| ATOM | 2778 | CG | HIS | 203 | 4.173 | −73.757 | 113.076 | 1.00 | 15.00 | B | C |
| ATOM | 2779 | CD2 | HIS | 203 | 3.148 | −73.977 | 112.221 | 1.00 | 15.47 | B | C |
| ATOM | 2780 | ND1 | HIS | 203 | 4.719 | −72.545 | 112.714 | 1.00 | 15.56 | B | N |
| ATOM | 2781 | CE1 | HIS | 203 | 4.058 | −72.056 | 111.681 | 1.00 | 15.06 | B | C |
| ATOM | 2782 | NE2 | HIS | 203 | 3.100 | −72.906 | 111.361 | 1.00 | 15.27 | B | N |
| ATOM | 2783 | C | HIS | 203 | 6.886 | −75.646 | 115.003 | 1.00 | 15.74 | B | C |
| ATOM | 2784 | O | HIS | 203 | 7.738 | −74.867 | 115.422 | 1.00 | 16.61 | B | O |
| ATOM | 2785 | N | ILE | 204 | 6.643 | −76.818 | 115.581 | 1.00 | 14.51 | B | N |
| ATOM | 2786 | CA | ILE | 204 | 7.421 | −77.263 | 116.734 | 1.00 | 15.77 | B | C |
| ATOM | 2787 | CB | ILE | 204 | 6.914 | −78.643 | 117.266 | 1.00 | 14.78 | B | C |
| ATOM | 2788 | CG2 | ILE | 204 | 7.885 | −79.192 | 118.305 | 1.00 | 14.01 | B | C |
| ATOM | 2789 | CG1 | ILE | 204 | 5.496 | −78.495 | 117.853 | 1.00 | 14.35 | B | C |
| ATOM | 2790 | CD1 | ILE | 204 | 4.849 | −79.812 | 118.317 | 1.00 | 11.99 | B | C |
| ATOM | 2791 | C | ILE | 204 | 8.890 | −77.404 | 116.303 | 1.00 | 15.99 | B | C |
| ATOM | 2792 | O | ILE | 204 | 9.803 | −76.963 | 117.001 | 1.00 | 14.96 | B | O |
| ATOM | 2793 | N | VAL | 205 | 9.108 | −78.011 | 115.140 | 1.00 | 16.33 | B | N |
| ATOM | 2794 | CA | VAL | 205 | 10.463 | −78.208 | 114.623 | 1.00 | 18.21 | B | C |
| ATOM | 2795 | CB | VAL | 205 | 10.455 | −79.139 | 113.402 | 1.00 | 17.84 | B | C |
| ATOM | 2796 | CG1 | VAL | 205 | 11.796 | −79.058 | 112.671 | 1.00 | 17.90 | B | C |
| ATOM | 2797 | CG2 | VAL | 205 | 10.169 | −80.565 | 113.862 | 1.00 | 18.55 | B | C |
| ATOM | 2798 | C | VAL | 205 | 11.153 | −76.906 | 114.242 | 1.00 | 17.83 | B | C |
| ATOM | 2799 | O | VAL | 205 | 12.317 | −76.698 | 114.550 | 1.00 | 17.35 | B | O |
| ATOM | 2800 | N | LEU | 206 | 10.434 | −76.027 | 113.565 | 1.00 | 18.27 | B | N |
| ATOM | 2801 | CA | LEU | 206 | 11.006 | −74.745 | 113.167 | 1.00 | 19.32 | B | C |
| ATOM | 2802 | CB | LEU | 206 | 10.051 | −74.037 | 112.202 | 1.00 | 19.42 | B | C |
| ATOM | 2803 | CG | LEU | 206 | 10.452 | −73.904 | 110.727 | 1.00 | 21.56 | B | C |
| ATOM | 2804 | CD1 | LEU | 206 | 11.505 | −74.932 | 110.327 | 1.00 | 21.35 | B | C |
| ATOM | 2805 | CD2 | LEU | 206 | 9.196 | −74.031 | 109.890 | 1.00 | 21.44 | B | C |
| ATOM | 2806 | C | LEU | 206 | 11.318 | −73.827 | 114.357 | 1.00 | 18.20 | B | C |
| ATOM | 2807 | O | LEU | 206 | 12.121 | −72.902 | 114.238 | 1.00 | 17.70 | B | O |
| ATOM | 2808 | N | ASN | 207 | 10.688 | −74.079 | 115.500 | 1.00 | 17.51 | B | N |
| ATOM | 2809 | CA | ASN | 207 | 10.918 | −73.251 | 116.680 | 1.00 | 17.40 | B | C |
| ATOM | 2810 | CB | ASN | 207 | 10.124 | −73.774 | 117.874 | 1.00 | 15.99 | B | C |
| ATOM | 2811 | CG | ASN | 207 | 10.184 | −72.828 | 119.073 | 1.00 | 16.60 | B | C |
| ATOM | 2812 | OD1 | ASN | 207 | 10.860 | −73.101 | 120.068 | 1.00 | 15.13 | B | O |
| ATOM | 2813 | ND2 | ASN | 207 | 9.485 | −71.701 | 118.970 | 1.00 | 13.67 | B | N |
| ATOM | 2814 | C | ASN | 207 | 12.387 | −73.144 | 117.061 | 1.00 | 18.07 | B | C |
| ATOM | 2815 | O | ASN | 207 | 12.804 | −72.133 | 117.621 | 1.00 | 17.57 | B | O |
| ATOM | 2816 | N | THR | 208 | 13.172 | −74.179 | 116.764 | 1.00 | 18.22 | B | N |
| ATOM | 2817 | CA | THR | 208 | 14.595 | −74.147 | 117.080 | 1.00 | 20.72 | B | C |
| ATOM | 2818 | CB | THR | 208 | 15.258 | −75.534 | 116.894 | 1.00 | 21.89 | B | C |
| ATOM | 2819 | OG1 | THR | 208 | 15.018 | −76.015 | 115.565 | 1.00 | 23.70 | B | O |
| ATOM | 2820 | CG2 | THR | 208 | 14.676 | −76.540 | 117.906 | 1.00 | 22.78 | B | C |
| ATOM | 2821 | C | THR | 208 | 15.366 | −73.105 | 116.268 | 1.00 | 20.46 | B | C |
| ATOM | 2822 | O | THR | 208 | 16.501 | −72.785 | 116.599 | 1.00 | 21.62 | B | O |
| ATOM | 2823 | N | THR | 209 | 14.760 | −72.568 | 115.212 | 1.00 | 19.69 | B | N |
| ATOM | 2824 | CA | THR | 209 | 15.423 | −71.536 | 114.413 | 1.00 | 19.16 | B | C |
| ATOM | 2825 | CB | THR | 209 | 15.153 | −71.700 | 112.899 | 1.00 | 18.90 | B | C |
| ATOM | 2826 | OG1 | THR | 209 | 13.776 | −71.401 | 112.619 | 1.00 | 17.30 | B | O |
| ATOM | 2827 | CG2 | THR | 209 | 15.474 | −73.121 | 112.451 | 1.00 | 18.39 | B | C |
| ATOM | 2828 | C | THR | 209 | 14.944 | −70.134 | 114.804 | 1.00 | 19.06 | B | C |
| ATOM | 2829 | O | THR | 209 | 15.456 | −69.144 | 114.299 | 1.00 | 17.85 | B | O |
| ATOM | 2830 | N | PHE | 210 | 13.962 | −70.053 | 115.698 | 1.00 | 18.89 | B | N |
| ATOM | 2831 | CA | PHE | 210 | 13.429 | −68.760 | 116.112 | 1.00 | 20.27 | B | C |
| ATOM | 2832 | CB | PHE | 210 | 12.107 | −68.936 | 116.877 | 1.00 | 18.70 | B | C |
| ATOM | 2833 | CG | PHE | 210 | 11.307 | −67.661 | 117.020 | 1.00 | 18.90 | B | C |
| ATOM | 2834 | CD1 | PHE | 210 | 10.694 | −67.081 | 115.914 | 1.00 | 18.35 | B | C |
| ATOM | 2835 | CD2 | PHE | 210 | 11.161 | −67.046 | 118.263 | 1.00 | 19.05 | B | C |
| ATOM | 2836 | CE1 | PHE | 210 | 9.945 | −65.909 | 116.039 | 1.00 | 18.54 | B | C |
| ATOM | 2837 | CE2 | PHE | 210 | 10.409 | −65.865 | 118.401 | 1.00 | 19.42 | B | C |
| ATOM | 2838 | CZ | PHE | 210 | 9.802 | −65.299 | 117.286 | 1.00 | 18.58 | B | C |
| ATOM | 2839 | C | PHE | 210 | 14.411 | −68.002 | 116.986 | 1.00 | 20.77 | B | C |
| ATOM | 2840 | O | PHE | 210 | 14.847 | −68.498 | 118.021 | 1.00 | 20.70 | B | O |
| ATOM | 2841 | N | CYS | 211 | 14.759 | −66.794 | 116.565 | 1.00 | 22.21 | B | N |
| ATOM | 2842 | CA | CYS | 211 | 15.674 | −65.953 | 117.327 | 1.00 | 24.26 | B | C |
| ATOM | 2843 | CB | CYS | 211 | 16.575 | −65.155 | 116.391 | 1.00 | 24.98 | B | C |
| ATOM | 2844 | SG | CYS | 211 | 17.664 | −64.026 | 117.267 | 1.00 | 27.94 | B | S |
| ATOM | 2845 | C | CYS | 211 | 14.824 | −64.996 | 118.148 | 1.00 | 25.17 | B | C |
| ATOM | 2846 | O | CYS | 211 | 14.060 | −64.209 | 117.586 | 1.00 | 24.50 | B | O |
| ATOM | 2847 | N | LEU | 212 | 14.950 | −65.076 | 119.471 | 1.00 | 25.78 | B | N |
| ATOM | 2848 | CA | LEU | 212 | 14.173 | −64.229 | 120.375 | 1.00 | 27.77 | B | C |
| ATOM | 2849 | CB | LEU | 212 | 14.396 | −64.652 | 121.830 | 1.00 | 26.74 | B | C |
| ATOM | 2850 | CG | LEU | 212 | 13.735 | −65.968 | 122.237 | 1.00 | 26.59 | B | C |
| ATOM | 2851 | CD1 | LEU | 212 | 14.046 | −66.279 | 123.700 | 1.00 | 26.25 | B | C |
| ATOM | 2852 | CD2 | LEU | 212 | 12.235 | −65.863 | 122.014 | 1.00 | 24.89 | B | C |
| ATOM | 2853 | C | LEU | 212 | 14.448 | −62.739 | 120.253 | 1.00 | 28.72 | B | C |
| ATOM | 2854 | O | LEU | 212 | 13.521 | −61.931 | 120.271 | 1.00 | 29.38 | B | O |
| ATOM | 2855 | N | GLN | 213 | 15.720 | −62.379 | 120.140 | 1.00 | 30.42 | B | N |
| ATOM | 2856 | CA | GLN | 213 | 16.108 | −60.982 | 120.027 | 1.00 | 32.34 | B | C |
| ATOM | 2857 | CB | GLN | 213 | 17.631 | −60.869 | 119.893 | 1.00 | 35.12 | B | C |
| ATOM | 2858 | CG | GLN | 213 | 18.122 | −59.462 | 119.576 | 1.00 | 38.96 | B | C |
| ATOM | 2859 | CD | GLN | 213 | 18.458 | −58.655 | 120.817 | 1.00 | 41.53 | B | C |
| ATOM | 2860 | OE1 | GLN | 213 | 19.566 | −58.754 | 121.357 | 1.00 | 42.98 | B | O |
| ATOM | 2861 | NE2 | GLN | 213 | 17.500 | −57.858 | 121.284 | 1.00 | 42.35 | B | N |
| ATOM | 2862 | C | GLN | 213 | 15.453 | −60.262 | 118.846 | 1.00 | 32.03 | B | C |
| ATOM | 2863 | O | GLN | 213 | 15.005 | −59.126 | 118.986 | 1.00 | 32.80 | B | O |
| ATOM | 2864 | N | THR | 214 | 15.390 | −60.919 | 117.691 | 1.00 | 30.84 | B | N |
| ATOM | 2865 | CA | THR | 214 | 14.828 | −60.288 | 116.497 | 1.00 | 29.82 | B | C |
| ATOM | 2866 | CB | THR | 214 | 15.808 | −60.393 | 115.316 | 1.00 | 29.30 | B | C |
| ATOM | 2867 | OG1 | THR | 214 | 16.083 | −61.773 | 115.049 | 1.00 | 28.95 | B | O |
| ATOM | 2868 | CG2 | THR | 214 | 17.108 | −59.679 | 115.636 | 1.00 | 30.14 | B | C |
| ATOM | 2869 | C | THR | 214 | 13.472 | −60.792 | 116.009 | 1.00 | 29.36 | B | C |
| ATOM | 2870 | O | THR | 214 | 12.941 | −60.264 | 115.032 | 1.00 | 28.67 | B | O |
| ATOM | 2871 | N | GLN | 215 | 12.918 | −61.805 | 116.670 | 1.00 | 28.97 | B | N |
| ATOM | 2872 | CA | GLN | 215 | 11.623 | −62.361 | 116.273 | 1.00 | 29.50 | B | C |
| ATOM | 2873 | CB | GLN | 215 | 10.533 | −61.284 | 116.375 | 1.00 | 30.88 | B | C |
| ATOM | 2874 | CG | GLN | 215 | 10.336 | −60.704 | 117.764 | 1.00 | 33.91 | B | C |
| ATOM | 2875 | CD | GLN | 215 | 9.754 | −61.711 | 118.726 | 1.00 | 35.71 | B | C |
| ATOM | 2876 | OE1 | GLN | 215 | 8.651 | −62.221 | 118.513 | 1.00 | 37.61 | B | O |
| ATOM | 2877 | NE2 | GLN | 215 | 10.492 | −62.010 | 119.793 | 1.00 | 36.76 | B | N |
| ATOM | 2878 | C | GLN | 215 | 11.670 | −62.890 | 114.834 | 1.00 | 28.46 | B | C |
| ATOM | 2879 | O | GLN | 215 | 10.696 | −62.774 | 114.093 | 1.00 | 28.52 | B | O |
| ATOM | 2880 | N | ASN | 216 | 12.801 | −63.465 | 114.442 | 1.00 | 26.98 | B | N |
| ATOM | 2881 | CA | ASN | 216 | 12.960 | −63.990 | 113.092 | 1.00 | 25.74 | B | C |
| ATOM | 2882 | CB | ASN | 216 | 14.085 | −63.250 | 112.363 | 1.00 | 27.52 | B | C |
| ATOM | 2883 | CG | ASN | 216 | 13.770 | −61.786 | 112.093 | 1.00 | 28.70 | B | C |
| ATOM | 2884 | OD1 | ASN | 216 | 14.664 | −61.022 | 111.749 | 1.00 | 27.69 | B | O |
| ATOM | 2885 | ND2 | ASN | 216 | 12.503 | −61.394 | 112.228 | 1.00 | 29.92 | B | N |
| ATOM | 2886 | C | ASN | 216 | 13.326 | −65.461 | 113.125 | 1.00 | 24.08 | B | C |
| ATOM | 2887 | O | ASN | 216 | 13.849 | −65.945 | 114.119 | 1.00 | 22.97 | B | O |
| ATOM | 2888 | N | PHE | 217 | 13.049 | −66.164 | 112.032 | 1.00 | 22.85 | B | N |
| ATOM | 2889 | CA | PHE | 217 | 13.410 | −67.568 | 111.906 | 1.00 | 22.30 | B | C |
| ATOM | 2890 | CB | PHE | 217 | 12.323 | −68.373 | 111.195 | 1.00 | 20.84 | B | C |
| ATOM | 2891 | CG | PHE | 217 | 11.052 | −68.503 | 111.975 | 1.00 | 20.02 | B | C |
| ATOM | 2892 | CD1 | PHE | 217 | 10.095 | −67.494 | 111.948 | 1.00 | 19.19 | B | C |
| ATOM | 2893 | CD2 | PHE | 217 | 10.805 | −69.644 | 112.733 | 1.00 | 19.92 | B | C |
| ATOM | 2894 | CE1 | PHE | 217 | 8.909 | −67.622 | 112.664 | 1.00 | 18.54 | B | C |
| ATOM | 2895 | CE2 | PHE | 217 | 9.620 | −69.780 | 113.453 | 1.00 | 19.66 | B | C |
| ATOM | 2896 | CZ | PHE | 217 | 8.672 | −68.763 | 113.415 | 1.00 | 19.48 | B | C |
| ATOM | 2897 | C | PHE | 217 | 14.676 | −67.588 | 111.057 | 1.00 | 22.98 | B | C |
| ATOM | 2898 | O | PHE | 217 | 14.652 | −67.184 | 109.895 | 1.00 | 21.70 | B | O |
| ATOM | 2899 | N | LEU | 218 | 15.777 | −68.046 | 111.645 | 1.00 | 23.53 | B | N |
| ATOM | 2900 | CA | LEU | 218 | 17.066 | −68.114 | 110.957 | 1.00 | 24.87 | B | C |
| ATOM | 2901 | CB | LEU | 218 | 18.210 | −67.785 | 111.930 | 1.00 | 26.08 | B | C |
| ATOM | 2902 | CG | LEU | 218 | 18.380 | −66.339 | 112.436 | 1.00 | 27.99 | B | C |
| ATOM | 2903 | CD1 | LEU | 218 | 17.118 | −65.845 | 113.126 | 1.00 | 27.86 | B | C |
| ATOM | 2904 | CD2 | LEU | 218 | 19.555 | −66.279 | 113.413 | 1.00 | 27.96 | B | C |
| ATOM | 2905 | C | LEU | 218 | 17.260 | −69.510 | 110.383 | 1.00 | 25.07 | B | C |
| ATOM | 2906 | O | LEU | 218 | 17.539 | −70.458 | 111.115 | 1.00 | 25.64 | B | O |
| ATOM | 2907 | N | CYS | 219 | 17.105 | −69.631 | 109.069 | 1.00 | 24.23 | B | N |
| ATOM | 2908 | CA | CYS | 219 | 17.244 | −70.918 | 108.396 | 1.00 | 23.85 | B | C |
| ATOM | 2909 | CB | CYS | 219 | 15.935 | −71.263 | 107.674 | 1.00 | 22.48 | B | C |
| ATOM | 2910 | SG | CYS | 219 | 14.462 | −71.225 | 108.747 | 1.00 | 19.92 | B | S |
| ATOM | 2911 | C | CYS | 219 | 18.406 | −70.868 | 107.398 | 1.00 | 24.42 | B | C |
| ATOM | 2912 | O | CYS | 219 | 18.231 | −70.505 | 106.227 | 1.00 | 22.82 | B | O |
| ATOM | 2913 | N | GLY | 220 | 19.590 | −71.252 | 107.870 | 1.00 | 25.23 | B | N |
| ATOM | 2914 | CA | GLY | 220 | 20.764 | −71.214 | 107.021 | 1.00 | 24.90 | B | C |
| ATOM | 2915 | C | GLY | 220 | 20.996 | −69.754 | 106.695 | 1.00 | 24.77 | B | C |
| ATOM | 2916 | O | GLY | 220 | 21.031 | −68.922 | 107.601 | 1.00 | 24.69 | B | O |
| ATOM | 2917 | N | PRO | 221 | 21.152 | −69.401 | 105.412 | 1.00 | 24.50 | B | N |
| ATOM | 2918 | CD | PRO | 221 | 21.305 | −70.265 | 104.229 | 1.00 | 23.93 | B | C |
| ATOM | 2919 | CA | PRO | 221 | 21.374 | −67.993 | 105.060 | 1.00 | 24.42 | B | C |
| ATOM | 2920 | CB | PRO | 221 | 22.053 | −68.090 | 103.699 | 1.00 | 23.72 | B | C |
| ATOM | 2921 | CG | PRO | 221 | 21.356 | −69.247 | 103.085 | 1.00 | 23.75 | B | C |
| ATOM | 2922 | C | PRO | 221 | 20.073 | −67.181 | 104.995 | 1.00 | 23.85 | B | C |
| ATOM | 2923 | O | PRO | 221 | 20.108 | −65.967 | 104.811 | 1.00 | 23.61 | B | O |
| ATOM | 2924 | N | LEU | 222 | 18.935 | −67.858 | 105.153 | 1.00 | 22.94 | B | N |
| ATOM | 2925 | CA | LEU | 222 | 17.627 | −67.209 | 105.084 | 1.00 | 22.55 | B | C |
| ATOM | 2926 | CB | LEU | 222 | 16.595 | −68.162 | 104.478 | 1.00 | 20.79 | B | C |
| ATOM | 2927 | CG | LEU | 222 | 16.913 | −68.732 | 103.100 | 1.00 | 19.76 | B | C |
| ATOM | 2928 | CD1 | LEU | 222 | 15.742 | −69.560 | 102.623 | 1.00 | 20.37 | B | C |
| ATOM | 2929 | CD2 | LEU | 222 | 17.199 | −67.594 | 102.122 | 1.00 | 20.15 | B | C |
| ATOM | 2930 | C | LEU | 222 | 17.093 | −66.695 | 106.415 | 1.00 | 22.84 | B | C |
| ATOM | 2931 | O | LEU | 222 | 17.341 | −67.275 | 107.473 | 1.00 | 23.24 | B | O |
| ATOM | 2932 | N | ARG | 223 | 16.343 | −65.603 | 106.333 | 1.00 | 22.89 | B | N |
| ATOM | 2933 | CA | ARG | 223 | 15.733 | −64.960 | 107.485 | 1.00 | 24.01 | B | C |
| ATOM | 2934 | CB | ARG | 223 | 16.451 | −63.650 | 107.782 | 1.00 | 26.47 | B | C |
| ATOM | 2935 | CG | ARG | 223 | 15.718 | −62.751 | 108.768 | 1.00 | 31.77 | B | C |
| ATOM | 2936 | CD | ARG | 223 | 16.177 | −61.310 | 108.616 | 1.00 | 35.27 | B | C |
| ATOM | 2937 | NE | ARG | 223 | 15.723 | −60.734 | 107.349 | 1.00 | 38.31 | B | N |
| ATOM | 2938 | CZ | ARG | 223 | 16.314 | −59.714 | 106.740 | 1.00 | 39.39 | B | C |
| ATOM | 2939 | NH1 | ARG | 223 | 17.389 | −59.158 | 107.279 | 1.00 | 41.22 | B | N |
| ATOM | 2940 | NH2 | ARG | 223 | 15.824 | −59.240 | 105.600 | 1.00 | 40.79 | B | N |
| ATOM | 2941 | C | ARG | 223 | 14.265 | −64.663 | 107.183 | 1.00 | 23.59 | B | C |
| ATOM | 2942 | O | ARG | 223 | 13.964 | −63.869 | 106.287 | 1.00 | 22.38 | B | O |
| ATOM | 2943 | N | TYR | 224 | 13.359 | −65.296 | 107.930 | 1.00 | 22.01 | B | N |
| ATOM | 2944 | CA | TYR | 224 | 11.921 | −65.086 | 107.742 | 1.00 | 21.48 | B | C |
| ATOM | 2945 | CB | TYR | 224 | 11.163 | −66.411 | 107.718 | 1.00 | 20.04 | B | C |
| ATOM | 2946 | CG | TYR | 224 | 11.572 | −67.355 | 106.615 | 1.00 | 19.13 | B | C |
| ATOM | 2947 | CD1 | TYR | 224 | 12.511 | −68.361 | 106.851 | 1.00 | 17.91 | B | C |
| ATOM | 2948 | CE1 | TYR | 224 | 12.886 | −69.240 | 105.848 | 1.00 | 18.05 | B | C |
| ATOM | 2949 | CD2 | TYR | 224 | 11.019 | −67.249 | 105.338 | 1.00 | 17.45 | B | C |
| ATOM | 2950 | CE2 | TYR | 224 | 11.392 | −68.124 | 104.319 | 1.00 | 17.93 | B | C |
| ATOM | 2951 | CZ | TYR | 224 | 12.328 | −69.118 | 104.587 | 1.00 | 17.71 | B | C |
| ATOM | 2952 | OH | TYR | 224 | 12.714 | −69.995 | 103.606 | 1.00 | 17.71 | B | O |
| ATOM | 2953 | C | TYR | 224 | 11.352 | −64.239 | 108.872 | 1.00 | 21.91 | B | C |
| ATOM | 2954 | O | TYR | 224 | 11.619 | −64.502 | 110.043 | 1.00 | 21.72 | B | O |
| ATOM | 2955 | N | THR | 225 | 10.556 | −63.238 | 108.509 | 1.00 | 21.54 | B | N |
| ATOM | 2956 | CA | THR | 225 | 9.942 | −62.340 | 109.478 | 1.00 | 21.65 | B | C |
| ATOM | 2957 | CB | THR | 225 | 10.335 | −60.874 | 109.202 | 1.00 | 23.19 | B | C |
| ATOM | 2958 | OG1 | THR | 225 | 9.847 | −60.492 | 107.912 | 1.00 | 22.96 | B | O |
| ATOM | 2959 | CG2 | THR | 225 | 11.853 | −60.695 | 109.232 | 1.00 | 23.33 | B | C |
| ATOM | 2960 | C | THR | 225 | 8.418 | −62.421 | 109.416 | 1.00 | 20.84 | B | C |
| ATOM | 2961 | O | THR | 225 | 7.849 | −63.042 | 108.513 | 1.00 | 19.99 | B | O |
| ATOM | 2962 | N | ILE | 226 | 7.764 | −61.773 | 110.376 | 1.00 | 19.42 | B | N |
| ATOM | 2963 | CA | ILE | 226 | 6.311 | −61.746 | 110.430 | 1.00 | 18.00 | B | C |
| ATOM | 2964 | CB | ILE | 226 | 5.837 | −61.101 | 111.768 | 1.00 | 17.71 | B | C |
| ATOM | 2965 | CG2 | ILE | 226 | 6.223 | −59.625 | 111.813 | 1.00 | 16.28 | B | C |
| ATOM | 2966 | CG1 | ILE | 226 | 4.334 | −61.306 | 111.963 | 1.00 | 15.92 | B | C |
| ATOM | 2967 | CD1 | ILE | 226 | 3.849 | −60.925 | 113.363 | 1.00 | 13.01 | B | C |
| ATOM | 2968 | C | ILE | 226 | 5.762 | −60.988 | 109.207 | 1.00 | 18.53 | B | C |
| ATOM | 2969 | O | ILE | 226 | 4.646 | −61.249 | 108.754 | 1.00 | 17.76 | B | O |
| ATOM | 2970 | N | GLU | 227 | 6.555 | −60.071 | 108.649 | 1.00 | 18.32 | B | N |
| ATOM | 2971 | CA | GLU | 227 | 6.119 | −59.335 | 107.468 | 1.00 | 19.11 | B | C |
| ATOM | 2972 | CB | GLU | 227 | 7.110 | −58.221 | 107.103 | 1.00 | 20.59 | B | C |
| ATOM | 2973 | CG | GLU | 227 | 7.032 | −56.939 | 107.959 | 1.00 | 20.58 | B | C |
| ATOM | 2974 | CD | GLU | 227 | 7.477 | −57.143 | 109.396 | 1.00 | 21.61 | B | C |
| ATOM | 2975 | OE1 | GLU | 227 | 8.490 | −57.839 | 109.617 | 1.00 | 23.10 | B | O |
| ATOM | 2976 | OE2 | GLU | 227 | 6.825 | −56.596 | 110.308 | 1.00 | 22.37 | B | O |
| ATOM | 2977 | C | GLU | 227 | 5.960 | −60.277 | 106.273 | 1.00 | 18.86 | B | C |
| ATOM | 2978 | O | GLU | 227 | 5.114 | −60.049 | 105.416 | 1.00 | 18.23 | B | O |
| ATOM | 2979 | N | ASP | 228 | 6.767 | −61.334 | 106.199 | 1.00 | 18.37 | B | N |
| ATOM | 2980 | CA | ASP | 228 | 6.631 | −62.255 | 105.071 | 1.00 | 18.69 | B | C |
| ATOM | 2981 | CB | ASP | 228 | 7.733 | −63.333 | 105.088 | 1.00 | 18.93 | B | C |
| ATOM | 2982 | CG | ASP | 228 | 9.135 | −62.743 | 104.950 | 1.00 | 19.98 | B | C |
| ATOM | 2983 | OD1 | ASP | 228 | 9.348 | −61.929 | 104.031 | 1.00 | 20.95 | B | O |
| ATOM | 2984 | OD2 | ASP | 228 | 10.025 | −63.094 | 105.754 | 1.00 | 19.87 | B | O |
| ATOM | 2985 | C | ASP | 228 | 5.239 | −62.899 | 105.102 | 1.00 | 18.01 | B | C |
| ATOM | 2986 | O | ASP | 228 | 4.593 | −63.045 | 104.064 | 1.00 | 18.44 | B | O |
| ATOM | 2987 | N | GLY | 229 | 4.776 | −63.281 | 106.289 | 1.00 | 17.20 | B | N |
| ATOM | 2988 | CA | GLY | 229 | 3.449 | −63.866 | 106.392 | 1.00 | 17.52 | B | C |
| ATOM | 2989 | C | GLY | 229 | 2.371 | −62.845 | 106.035 | 1.00 | 17.70 | B | C |
| ATOM | 2990 | O | GLY | 229 | 1.411 | −63.157 | 105.330 | 1.00 | 18.08 | B | O |
| ATOM | 2991 | N | ALA | 230 | 2.532 | −61.614 | 106.510 | 1.00 | 17.31 | B | N |
| ATOM | 2992 | CA | ALA | 230 | 1.561 | −60.557 | 106.237 | 1.00 | 17.67 | B | C |
| ATOM | 2993 | CB | ALA | 230 | 1.921 | −59.312 | 107.031 | 1.00 | 17.60 | B | C |
| ATOM | 2994 | C | ALA | 230 | 1.457 | −60.213 | 104.749 | 1.00 | 18.51 | B | C |
| ATOM | 2995 | O | ALA | 230 | 0.367 | −59.982 | 104.231 | 1.00 | 17.21 | B | O |
| ATOM | 2996 | N | ARG | 231 | 2.594 | −60.177 | 104.060 | 1.00 | 19.86 | B | N |
| ATOM | 2997 | CA | ARG | 231 | 2.599 | −59.844 | 102.643 | 1.00 | 20.24 | B | C |
| ATOM | 2998 | CB | ARG | 231 | 4.027 | −59.549 | 102.173 | 1.00 | 21.54 | B | C |
| ATOM | 2999 | CG | ARG | 231 | 4.665 | −58.322 | 102.843 | 1.00 | 23.12 | B | C |
| ATOM | 3000 | CD | ARG | 231 | 3.911 | −57.026 | 102.506 | 1.00 | 23.94 | B | C |
| ATOM | 3001 | NE | ARG | 231 | 4.022 | −56.665 | 101.092 | 1.00 | 25.20 | B | N |
| ATOM | 3002 | CZ | ARG | 231 | 5.058 | −56.026 | 100.553 | 1.00 | 25.86 | B | C |
| ATOM | 3003 | NH1 | ARG | 231 | 6.090 | −55.661 | 101.301 | 1.00 | 25.92 | B | N |
| ATOM | 3004 | NH2 | ARG | 231 | 5.064 | −55.746 | 99.258 | 1.00 | 26.68 | B | N |
| ATOM | 3005 | C | ARG | 231 | 1.963 | −60.919 | 101.765 | 1.00 | 20.12 | B | C |
| ATOM | 3006 | O | ARG | 231 | 1.561 | −60.632 | 100.640 | 1.00 | 19.52 | B | O |
| ATOM | 3007 | N | VAL | 232 | 1.865 | −62.153 | 102.256 | 1.00 | 19.90 | B | N |
| ATOM | 3008 | CA | VAL | 232 | 1.239 | −63.195 | 101.450 | 1.00 | 19.34 | B | C |
| ATOM | 3009 | CB | VAL | 232 | 1.927 | −64.592 | 101.612 | 1.00 | 20.66 | B | C |
| ATOM | 3010 | CG1 | VAL | 232 | 3.413 | −64.476 | 101.307 | 1.00 | 19.33 | B | C |
| ATOM | 3011 | CG2 | VAL | 232 | 1.671 | −65.169 | 103.017 | 1.00 | 18.79 | B | C |
| ATOM | 3012 | C | VAL | 232 | −0.245 | −63.330 | 101.779 | 1.00 | 19.07 | B | C |
| ATOM | 3013 | O | VAL | 232 | −0.926 | −64.191 | 101.230 | 1.00 | 17.55 | B | O |
| ATOM | 3014 | N | GLY | 233 | −0.745 | −62.486 | 102.680 | 1.00 | 19.78 | B | N |
| ATOM | 3015 | CA | GLY | 233 | −2.161 | −62.535 | 103.008 | 1.00 | 20.41 | B | C |
| ATOM | 3016 | C | GLY | 233 | −2.643 | −62.915 | 104.397 | 1.00 | 21.13 | B | C |
| ATOM | 3017 | O | GLY | 233 | −3.805 | −62.655 | 104.719 | 1.00 | 21.49 | B | O |
| ATOM | 3018 | N | PHE | 234 | −1.806 | −63.541 | 105.223 | 1.00 | 20.51 | B | N |
| ATOM | 3019 | CA | PHE | 234 | −2.268 | −63.905 | 106.563 | 1.00 | 20.56 | B | C |
| ATOM | 3020 | CB | PHE | 234 | −1.205 | −64.706 | 107.326 | 1.00 | 20.73 | B | C |
| ATOM | 3021 | CG | PHE | 234 | −0.998 | −66.103 | 106.810 | 1.00 | 20.24 | B | C |
| ATOM | 3022 | CD1 | PHE | 234 | 0.169 | −66.441 | 106.135 | 1.00 | 19.02 | B | C |
| ATOM | 3023 | CD2 | PHE | 234 | −1.956 | −67.093 | 107.035 | 1.00 | 19.67 | B | C |
| ATOM | 3024 | CE1 | PHE | 234 | 0.386 | −67.747 | 105.695 | 1.00 | 19.63 | B | C |
| ATOM | 3025 | CE2 | PHE | 234 | −1.748 | −68.403 | 106.597 | 1.00 | 19.47 | B | C |
| ATOM | 3026 | CZ | PHE | 234 | −0.574 | −68.731 | 105.928 | 1.00 | 18.87 | B | C |
| ATOM | 3027 | C | PHE | 234 | −2.602 | −62.651 | 107.362 | 1.00 | 20.67 | B | C |
| ATOM | 3028 | O | PHE | 234 | −1.936 | −61.624 | 107.234 | 1.00 | 20.71 | B | O |
| ATOM | 3029 | N | GLN | 235 | −3.632 | −62.740 | 108.194 | 1.00 | 21.02 | B | N |
| ATOM | 3030 | CA | GLN | 235 | −4.051 | −61.614 | 109.020 | 1.00 | 22.09 | B | C |
| ATOM | 3031 | CB | GLN | 235 | −5.468 | −61.845 | 109.548 | 1.00 | 22.57 | B | C |
| ATOM | 3032 | CG | GLN | 235 | −6.481 | −61.997 | 108.428 | 1.00 | 24.45 | B | C |
| ATOM | 3033 | CD | GLN | 235 | −7.898 | −62.104 | 108.926 | 1.00 | 27.02 | B | C |
| ATOM | 3034 | OE1 | GLN | 235 | −8.219 | −62.966 | 109.748 | 1.00 | 27.06 | B | O |
| ATOM | 3035 | NE2 | GLN | 235 | −8.768 | −61.229 | 108.423 | 1.00 | 27.49 | B | N |
| ATOM | 3036 | C | GLN | 235 | −3.085 | −61.414 | 110.173 | 1.00 | 21.61 | B | C |
| ATOM | 3037 | O | GLN | 235 | −2.546 | −62.372 | 110.718 | 1.00 | 21.23 | B | O |
| ATOM | 3038 | N | VAL | 236 | −2.876 | −60.158 | 110.542 | 1.00 | 21.74 | B | N |
| ATOM | 3039 | CA | VAL | 236 | −1.957 | −59.813 | 111.616 | 1.00 | 22.27 | B | C |
| ATOM | 3040 | CB | VAL | 236 | −1.915 | −58.278 | 111.810 | 1.00 | 22.19 | B | C |
| ATOM | 3041 | CG1 | VAL | 236 | −1.086 | −57.914 | 113.032 | 1.00 | 22.00 | B | C |
| ATOM | 3042 | CG2 | VAL | 236 | −1.329 | −57.628 | 110.562 | 1.00 | 21.53 | B | C |
| ATOM | 3043 | C | VAL | 236 | −2.250 | −60.503 | 112.944 | 1.00 | 22.51 | B | C |
| ATOM | 3044 | O | VAL | 236 | −1.330 | −60.979 | 113.604 | 1.00 | 22.18 | B | O |
| ATOM | 3045 | N | GLU | 237 | −3.521 | −60.558 | 113.332 | 1.00 | 22.41 | B | N |
| ATOM | 3046 | CA | GLU | 237 | −3.928 | −61.198 | 114.586 | 1.00 | 22.96 | B | C |
| ATOM | 3047 | CB | GLU | 237 | −5.452 | −61.107 | 114.748 | 1.00 | 25.41 | B | C |
| ATOM | 3048 | CG | GLU | 237 | −6.012 | −61.912 | 115.905 | 1.00 | 29.15 | B | C |
| ATOM | 3049 | CD | GLU | 237 | −7.516 | −61.721 | 116.076 | 1.00 | 31.86 | B | C |
| ATOM | 3050 | OE1 | GLU | 237 | −8.245 | −61.756 | 115.062 | 1.00 | 32.35 | B | O |
| ATOM | 3051 | OE2 | GLU | 237 | −7.972 | −61.545 | 117.227 | 1.00 | 32.72 | B | O |
| ATOM | 3052 | C | GLU | 237 | −3.488 | −62.659 | 114.628 | 1.00 | 21.89 | B | C |
| ATOM | 3053 | O | GLU | 237 | −2.981 | −63.141 | 115.643 | 1.00 | 20.82 | B | O |
| ATOM | 3054 | N | PHE | 238 | −3.698 | −63.359 | 113.516 | 1.00 | 20.52 | B | N |
| ATOM | 3055 | CA | PHE | 238 | −3.303 | −64.753 | 113.394 | 1.00 | 19.64 | B | C |
| ATOM | 3056 | CB | PHE | 238 | −3.746 | −65.287 | 112.033 | 1.00 | 18.50 | B | C |
| ATOM | 3057 | CG | PHE | 238 | −3.147 | −66.608 | 111.679 | 1.00 | 18.24 | B | C |
| ATOM | 3058 | CD1 | PHE | 238 | −3.576 | −67.771 | 112.303 | 1.00 | 18.18 | B | C |
| ATOM | 3059 | CD2 | PHE | 238 | −2.133 | −66.689 | 110.724 | 1.00 | 18.29 | B | C |
| ATOM | 3060 | CE1 | PHE | 238 | −3.008 | −69.001 | 111.983 | 1.00 | 18.90 | B | C |
| ATOM | 3061 | CE2 | PHE | 238 | −1.556 | −67.916 | 110.396 | 1.00 | 18.97 | B | C |
| ATOM | 3062 | CZ | PHE | 238 | −1.994 | −69.072 | 111.025 | 1.00 | 19.34 | B | C |
| ATOM | 3063 | C | PHE | 238 | −1.777 | −64.831 | 113.526 | 1.00 | 19.52 | B | C |
| ATOM | 3064 | O | PHE | 238 | −1.243 | −65.653 | 114.276 | 1.00 | 17.63 | B | O |
| ATOM | 3065 | N | LEU | 239 | −1.087 | −63.957 | 112.799 | 1.00 | 19.51 | B | N |
| ATOM | 3066 | CA | LEU | 239 | 0.372 | −63.903 | 112.840 | 1.00 | 21.27 | B | C |
| ATOM | 3067 | CB | LEU | 239 | 0.881 | −62.786 | 111.918 | 1.00 | 19.88 | B | C |
| ATOM | 3068 | CG | LEU | 239 | 1.358 | −63.090 | 110.485 | 1.00 | 21.98 | B | C |
| ATOM | 3069 | CD1 | LEU | 239 | 0.983 | −64.488 | 110.062 | 1.00 | 19.74 | B | C |
| ATOM | 3070 | CD2 | LEU | 239 | 0.798 | −62.046 | 109.521 | 1.00 | 20.05 | B | C |
| ATOM | 3071 | C | LEU | 239 | 0.876 | −63.678 | 114.268 | 1.00 | 21.93 | B | C |
| ATOM | 3072 | O | LEU | 239 | 1.819 | −64.347 | 114.711 | 1.00 | 21.20 | B | O |
| ATOM | 3073 | N | GLU | 240 | 0.244 | −62.752 | 114.991 | 1.00 | 22.27 | B | N |
| ATOM | 3074 | CA | GLU | 240 | 0.645 | −62.455 | 116.366 | 1.00 | 24.45 | B | C |
| ATOM | 3075 | CB | GLU | 240 | −0.195 | −61.313 | 116.960 | 1.00 | 26.77 | B | C |
| ATOM | 3076 | CG | GLU | 240 | −0.043 | −59.949 | 116.274 | 1.00 | 30.99 | B | C |
| ATOM | 3077 | CD | GLU | 240 | 1.383 | −59.403 | 116.310 | 1.00 | 34.68 | B | C |
| ATOM | 3078 | OE1 | GLU | 240 | 2.143 | −59.754 | 117.237 | 1.00 | 37.47 | B | O |
| ATOM | 3079 | OE2 | GLU | 240 | 1.743 | −58.604 | 115.417 | 1.00 | 36.81 | B | O |
| ATOM | 3080 | C | GLU | 240 | 0.511 | −63.689 | 117.254 | 1.00 | 23.48 | B | C |
| ATOM | 3081 | O | GLU | 240 | 1.373 | −63.952 | 118.084 | 1.00 | 23.70 | B | O |
| ATOM | 3082 | N | LEU | 241 | −0.580 | −64.430 | 117.087 | 1.00 | 22.32 | B | N |
| ATOM | 3083 | CA | LEU | 241 | −0.801 | −65.643 | 117.864 | 1.00 | 21.98 | B | C |
| ATOM | 3084 | CB | LEU | 241 | −2.165 | −66.258 | 117.517 | 1.00 | 23.58 | B | C |
| ATOM | 3085 | CG | LEU | 241 | −2.573 | −67.588 | 118.172 | 1.00 | 25.77 | B | C |
| ATOM | 3086 | CD1 | LEU | 241 | −2.760 | −67.413 | 119.674 | 1.00 | 26.71 | B | C |
| ATOM | 3087 | CD2 | LEU | 241 | −3.869 | −68.080 | 117.550 | 1.00 | 27.49 | B | C |
| ATOM | 3088 | C | LEU | 241 | 0.316 | −66.639 | 117.544 | 1.00 | 20.98 | B | C |
| ATOM | 3089 | O | LEU | 241 | 0.858 | −67.281 | 118.434 | 1.00 | 20.13 | B | O |
| ATOM | 3090 | N | LEU | 242 | 0.666 | −66.746 | 116.265 | 1.00 | 19.66 | B | N |
| ATOM | 3091 | CA | LEU | 242 | 1.711 | −67.663 | 115.827 | 1.00 | 19.39 | B | C |
| ATOM | 3092 | CB | LEU | 242 | 1.757 | −67.706 | 114.296 | 1.00 | 17.57 | B | C |
| ATOM | 3093 | CG | LEU | 242 | 2.800 | −68.626 | 113.656 | 1.00 | 17.99 | B | C |
| ATOM | 3094 | CD1 | LEU | 242 | 2.679 | −70.043 | 114.206 | 1.00 | 17.20 | B | C |
| ATOM | 3095 | CD2 | LEU | 242 | 2.593 | −68.633 | 112.148 | 1.00 | 17.85 | B | C |
| ATOM | 3096 | C | LEU | 242 | 3.093 | −67.301 | 116.380 | 1.00 | 19.08 | B | C |
| ATOM | 3097 | O | LEU | 242 | 3.825 | −68.174 | 116.858 | 1.00 | 18.27 | B | O |
| ATOM | 3098 | N | PHE | 243 | 3.448 | −66.020 | 116.322 | 1.00 | 19.06 | B | N |
| ATOM | 3099 | CA | PHE | 243 | 4.746 | −65.587 | 116.820 | 1.00 | 20.50 | B | C |
| ATOM | 3100 | CB | PHE | 243 | 5.120 | −64.209 | 116.250 | 1.00 | 20.80 | B | C |
| ATOM | 3101 | CG | PHE | 243 | 5.719 | −64.280 | 114.866 | 1.00 | 21.15 | B | C |
| ATOM | 3102 | CD1 | PHE | 243 | 4.944 | −64.674 | 113.773 | 1.00 | 21.05 | B | C |
| ATOM | 3103 | CD2 | PHE | 243 | 7.071 | −64.015 | 114.665 | 1.00 | 20.91 | B | C |
| ATOM | 3104 | CE1 | PHE | 243 | 5.508 | −64.810 | 112.497 | 1.00 | 21.45 | B | C |
| ATOM | 3105 | CE2 | PHE | 243 | 7.646 | −64.149 | 113.395 | 1.00 | 21.14 | B | C |
| ATOM | 3106 | CZ | PHE | 243 | 6.858 | −64.549 | 112.308 | 1.00 | 21.10 | B | C |
| ATOM | 3107 | C | PHE | 243 | 4.821 | −65.589 | 118.340 | 1.00 | 21.66 | B | C |
| ATOM | 3108 | O | PHE | 243 | 5.905 | −65.741 | 118.904 | 1.00 | 21.34 | B | O |
| ATOM | 3109 | N | HIS | 244 | 3.673 | −65.424 | 118.995 | 1.00 | 22.31 | B | N |
| ATOM | 3110 | CA | HIS | 244 | 3.612 | −65.456 | 120.448 | 1.00 | 23.28 | B | C |
| ATOM | 3111 | CB | HIS | 244 | 2.220 | −65.048 | 120.951 | 1.00 | 26.24 | B | C |
| ATOM | 3112 | CG | HIS | 244 | 1.972 | −65.395 | 122.391 | 1.00 | 29.43 | B | C |
| ATOM | 3113 | CD2 | HIS | 244 | 2.047 | −64.644 | 123.516 | 1.00 | 30.00 | B | C |
| ATOM | 3114 | ND1 | HIS | 244 | 1.623 | −66.666 | 122.804 | 1.00 | 30.79 | B | N |
| ATOM | 3115 | CE1 | HIS | 244 | 1.494 | −66.682 | 124.119 | 1.00 | 30.26 | B | C |
| ATOM | 3116 | NE2 | HIS | 244 | 1.747 | −65.469 | 124.575 | 1.00 | 30.77 | B | N |
| ATOM | 3117 | C | HIS | 244 | 3.904 | −66.892 | 120.857 | 1.00 | 21.80 | B | C |
| ATOM | 3118 | O | HIS | 244 | 4.636 | −67.133 | 121.809 | 1.00 | 21.67 | B | O |
| ATOM | 3119 | N | PHE | 245 | 3.308 | −67.837 | 120.137 | 1.00 | 19.90 | B | N |
| ATOM | 3120 | CA | PHE | 245 | 3.529 | −69.255 | 120.389 | 1.00 | 18.67 | B | C |
| ATOM | 3121 | CB | PHE | 245 | 2.775 | −70.104 | 119.356 | 1.00 | 18.04 | B | C |
| ATOM | 3122 | CG | PHE | 245 | 3.217 | −71.540 | 119.316 | 1.00 | 17.45 | B | C |
| ATOM | 3123 | CD1 | PHE | 245 | 2.802 | −72.439 | 120.294 | 1.00 | 16.43 | B | C |
| ATOM | 3124 | CD2 | PHE | 245 | 4.087 | −71.985 | 118.321 | 1.00 | 17.18 | B | C |
| ATOM | 3125 | CE1 | PHE | 245 | 3.244 | −73.760 | 120.286 | 1.00 | 16.09 | B | C |
| ATOM | 3126 | CE2 | PHE | 245 | 4.539 | −73.306 | 118.303 | 1.00 | 17.01 | B | C |
| ATOM | 3127 | CZ | PHE | 245 | 4.117 | −74.196 | 119.287 | 1.00 | 16.45 | B | C |
| ATOM | 3128 | C | PHE | 245 | 5.030 | −69.556 | 120.290 | 1.00 | 17.95 | B | C |
| ATOM | 3129 | O | PHE | 245 | 5.600 | −70.169 | 121.183 | 1.00 | 17.52 | B | O |
| ATOM | 3130 | N | HIS | 246 | 5.666 | −69.115 | 119.206 | 1.00 | 16.92 | B | N |
| ATOM | 3131 | CA | HIS | 246 | 7.092 | −69.365 | 119.024 | 1.00 | 16.61 | B | C |
| ATOM | 3132 | CB | HIS | 246 | 7.541 | −68.936 | 117.620 | 1.00 | 15.88 | B | C |
| ATOM | 3133 | CG | HIS | 246 | 7.208 | −69.935 | 116.554 | 1.00 | 16.90 | B | C |
| ATOM | 3134 | CD2 | HIS | 246 | 6.248 | −69.934 | 115.599 | 1.00 | 17.28 | B | C |
| ATOM | 3135 | ND1 | HIS | 246 | 7.856 | −71.145 | 116.440 | 1.00 | 16.47 | B | N |
| ATOM | 3136 | CE1 | HIS | 246 | 7.309 | −71.848 | 115.465 | 1.00 | 16.06 | B | C |
| ATOM | 3137 | NE2 | HIS | 246 | 6.330 | −71.136 | 114.940 | 1.00 | 15.84 | B | N |
| ATOM | 3138 | C | HIS | 246 | 7.959 | −68.704 | 120.095 | 1.00 | 16.14 | B | C |
| ATOM | 3139 | O | HIS | 246 | 8.895 | −69.317 | 120.598 | 1.00 | 16.15 | B | O |
| ATOM | 3140 | N | GLY | 247 | 7.666 | −67.461 | 120.445 | 1.00 | 15.88 | B | N |
| ATOM | 3141 | CA | GLY | 247 | 8.449 | −66.813 | 121.488 | 1.00 | 16.58 | B | C |
| ATOM | 3142 | C | GLY | 247 | 8.326 | −67.552 | 122.820 | 1.00 | 17.29 | B | C |
| ATOM | 3143 | O | GLY | 247 | 9.325 | −67.849 | 123.474 | 1.00 | 16.50 | B | O |
| ATOM | 3144 | N | THR | 248 | 7.097 | −67.872 | 123.215 | 1.00 | 17.62 | B | N |
| ATOM | 3145 | CA | THR | 248 | 6.854 | −68.569 | 124.473 | 1.00 | 19.52 | B | C |
| ATOM | 3146 | CB | THR | 248 | 5.334 | −68.737 | 124.715 | 1.00 | 20.24 | B | C |
| ATOM | 3147 | OG1 | THR | 248 | 4.694 | −67.462 | 124.589 | 1.00 | 19.56 | B | O |
| ATOM | 3148 | CG2 | THR | 248 | 5.065 | −69.288 | 126.110 | 1.00 | 20.09 | B | C |
| ATOM | 3149 | C | THR | 248 | 7.536 | −69.943 | 124.530 | 1.00 | 19.85 | B | C |
| ATOM | 3150 | O | THR | 248 | 8.183 | −70.282 | 125.519 | 1.00 | 18.96 | B | O |
| ATOM | 3151 | N | LEU | 249 | 7.392 | −70.728 | 123.466 | 1.00 | 19.93 | B | N |
| ATOM | 3152 | CA | LEU | 249 | 8.002 | −72.047 | 123.412 | 1.00 | 21.38 | B | C |
| ATOM | 3153 | CB | LEU | 249 | 7.552 | −72.793 | 122.145 | 1.00 | 21.21 | B | C |
| ATOM | 3154 | CG | LEU | 249 | 8.237 | −74.139 | 121.875 | 1.00 | 19.48 | B | C |
| ATOM | 3155 | CD1 | LEU | 249 | 7.922 | −75.090 | 123.015 | 1.00 | 21.12 | B | C |
| ATOM | 3156 | CD2 | LEU | 249 | 7.767 | −74.730 | 120.547 | 1.00 | 19.38 | B | C |
| ATOM | 3157 | C | LEU | 249 | 9.526 | −71.942 | 123.429 | 1.00 | 22.24 | B | C |
| ATOM | 3158 | O | LEU | 249 | 10.198 | −72.718 | 124.101 | 1.00 | 21.93 | B | O |
| ATOM | 3159 | N | ARG | 250 | 10.058 | −70.981 | 122.683 | 1.00 | 23.40 | B | N |
| ATOM | 3160 | CA | ARG | 250 | 11.499 | −70.759 | 122.592 | 1.00 | 25.09 | B | C |
| ATOM | 3161 | CB | ARG | 250 | 11.789 | −69.621 | 121.613 | 1.00 | 26.51 | B | C |
| ATOM | 3162 | CG | ARG | 250 | 12.705 | −69.985 | 120.465 | 1.00 | 28.87 | B | C |
| ATOM | 3163 | CD | ARG | 250 | 14.055 | −70.492 | 120.927 | 1.00 | 28.96 | B | C |
| ATOM | 3164 | NE | ARG | 250 | 15.002 | −70.513 | 119.817 | 1.00 | 29.63 | B | N |
| ATOM | 3165 | CZ | ARG | 250 | 16.291 | −70.825 | 119.918 | 1.00 | 31.64 | B | C |
| ATOM | 3166 | NH1 | ARG | 250 | 17.061 | −70.801 | 118.839 | 1.00 | 32.55 | B | N |
| ATOM | 3167 | NH2 | ARG | 250 | 16.813 | −71.170 | 121.087 | 1.00 | 32.97 | B | N |
| ATOM | 3168 | C | ARG | 250 | 12.139 | −70.411 | 123.932 | 1.00 | 25.34 | B | C |
| ATOM | 3169 | O | ARG | 250 | 13.191 | −70.942 | 124.285 | 1.00 | 24.47 | B | O |
| ATOM | 3170 | N | LYS | 251 | 11.508 | −69.503 | 124.667 | 1.00 | 26.27 | B | N |
| ATOM | 3171 | CA | LYS | 251 | 12.019 | −69.071 | 125.963 | 1.00 | 27.02 | B | C |
| ATOM | 3172 | CB | LYS | 251 | 11.126 | −67.966 | 126.536 | 1.00 | 27.26 | B | C |
| ATOM | 3173 | CG | LYS | 251 | 11.262 | −66.649 | 125.798 | 1.00 | 30.10 | B | C |
| ATOM | 3174 | CD | LYS | 251 | 10.253 | −65.601 | 126.269 | 1.00 | 32.49 | B | C |
| ATOM | 3175 | CE | LYS | 251 | 10.377 | −64.343 | 125.419 | 1.00 | 34.43 | B | C |
| ATOM | 3176 | NZ | LYS | 251 | 9.361 | −63.299 | 125.733 | 1.00 | 37.02 | B | N |
| ATOM | 3177 | C | LYS | 251 | 12.162 | −70.198 | 126.982 | 1.00 | 26.91 | B | C |
| ATOM | 3178 | O | LYS | 251 | 12.861 | −70.043 | 127.980 | 1.00 | 27.68 | B | O |
| ATOM | 3179 | N | LEU | 252 | 11.500 | −71.327 | 126.739 | 1.00 | 26.21 | B | N |
| ATOM | 3180 | CA | LEU | 252 | 11.577 | −72.462 | 127.648 | 1.00 | 25.54 | B | C |
| ATOM | 3181 | CB | LEU | 252 | 10.405 | −73.412 | 127.395 | 1.00 | 24.47 | B | C |
| ATOM | 3182 | CG | LEU | 252 | 9.028 | −72.881 | 127.809 | 1.00 | 23.94 | B | C |
| ATOM | 3183 | CD1 | LEU | 252 | 7.948 | −73.892 | 127.448 | 1.00 | 21.51 | B | C |
| ATOM | 3184 | CD2 | LEU | 252 | 9.026 | −72.598 | 129.317 | 1.00 | 22.42 | B | C |
| ATOM | 3185 | C | LEU | 252 | 12.904 | −73.224 | 127.552 | 1.00 | 26.17 | B | C |
| ATOM | 3186 | O | LEU | 252 | 13.200 | −74.077 | 128.399 | 1.00 | 25.06 | B | O |
| ATOM | 3187 | N | GLN | 253 | 13.696 | −72.920 | 126.525 | 1.00 | 26.13 | B | N |
| ATOM | 3188 | CA | GLN | 253 | 14.989 | −73.571 | 126.328 | 1.00 | 27.67 | B | C |
| ATOM | 3189 | CB | GLN | 253 | 15.986 | −73.124 | 127.406 | 1.00 | 29.23 | B | C |
| ATOM | 3190 | CG | GLN | 253 | 16.176 | −71.621 | 127.559 | 1.00 | 32.02 | B | C |
| ATOM | 3191 | CD | GLN | 253 | 17.160 | −71.282 | 128.673 | 1.00 | 34.60 | B | C |
| ATOM | 3192 | OE1 | GLN | 253 | 18.361 | −71.531 | 128.552 | 1.00 | 35.64 | B | O |
| ATOM | 3193 | NE2 | GLN | 253 | 16.651 | −70.725 | 129.770 | 1.00 | 35.92 | B | N |
| ATOM | 3194 | C | GLN | 253 | 14.859 | −75.091 | 126.392 | 1.00 | 27.27 | B | C |
| ATOM | 3195 | O | GLN | 253 | 15.553 | −75.740 | 127.170 | 1.00 | 27.62 | B | O |
| ATOM | 3196 | N | LEU | 254 | 13.979 | −75.659 | 125.578 | 1.00 | 26.25 | B | N |
| ATOM | 3197 | CA | LEU | 254 | 13.781 | −77.100 | 125.583 | 1.00 | 25.87 | B | C |
| ATOM | 3198 | CB | LEU | 254 | 12.541 | −77.484 | 124.763 | 1.00 | 23.25 | B | C |
| ATOM | 3199 | CG | LEU | 254 | 11.166 | −76.992 | 125.211 | 1.00 | 21.49 | B | C |
| ATOM | 3200 | CD1 | LEU | 254 | 10.100 | −77.661 | 124.364 | 1.00 | 20.44 | B | C |
| ATOM | 3201 | CD2 | LEU | 254 | 10.957 | −77.324 | 126.672 | 1.00 | 20.62 | B | C |
| ATOM | 3202 | C | LEU | 254 | 14.970 | −77.869 | 125.029 | 1.00 | 26.84 | B | C |
| ATOM | 3203 | O | LEU | 254 | 15.716 | −77.373 | 124.187 | 1.00 | 26.79 | B | O |
| ATOM | 3204 | N | GLN | 255 | 15.135 | −79.090 | 125.514 | 1.00 | 27.67 | B | N |
| ATOM | 3205 | CA | GLN | 255 | 16.191 | −79.964 | 125.037 | 1.00 | 29.47 | B | C |
| ATOM | 3206 | CB | GLN | 255 | 16.725 | −80.802 | 126.203 | 1.00 | 31.78 | B | C |
| ATOM | 3207 | CG | GLN | 255 | 17.397 | −79.913 | 127.263 | 1.00 | 36.11 | B | C |
| ATOM | 3208 | CD | GLN | 255 | 17.854 | −80.644 | 128.518 | 1.00 | 38.94 | B | C |
| ATOM | 3209 | OE1 | GLN | 255 | 18.321 | −80.015 | 129.476 | 1.00 | 40.55 | B | O |
| ATOM | 3210 | NE2 | GLN | 255 | 17.729 | −81.966 | 128.523 | 1.00 | 39.29 | B | N |
| ATOM | 3211 | C | GLN | 255 | 15.520 | −80.811 | 123.952 | 1.00 | 28.90 | B | C |
| ATOM | 3212 | O | GLN | 255 | 14.296 | −80.956 | 123.954 | 1.00 | 28.37 | B | O |
| ATOM | 3213 | N | GLU | 256 | 16.297 | −81.341 | 123.013 | 1.00 | 28.82 | B | N |
| ATOM | 3214 | CA | GLU | 256 | 15.719 | −82.122 | 121.922 | 1.00 | 29.56 | B | C |
| ATOM | 3215 | CB | CLU | 256 | 16.806 | −82.818 | 121.102 | 1.00 | 30.58 | B | C |
| ATOM | 3216 | CG | GLU | 256 | 17.589 | −81.895 | 120.205 | 1.00 | 32.90 | B | C |
| ATOM | 3217 | CD | GLU | 256 | 18.390 | −82.652 | 119.163 | 1.00 | 34.07 | B | C |
| ATOM | 3218 | OE1 | GLU | 256 | 19.015 | −83.674 | 119.514 | 1.00 | 35.05 | B | O |
| ATOM | 3219 | OE2 | GLU | 256 | 18.402 | −82.219 | 117.995 | 1.00 | 35.39 | B | O |
| ATOM | 3220 | C | GLU | 256 | 14.658 | −83.154 | 122.274 | 1.00 | 29.06 | B | C |
| ATOM | 3221 | O | GLU | 256 | 13.587 | −83.160 | 121.677 | 1.00 | 29.72 | B | O |
| ATOM | 3222 | N | PRO | 257 | 14.935 | −84.051 | 123.232 | 1.00 | 28.49 | B | N |
| ATOM | 3223 | CD | PRO | 257 | 16.157 | −84.286 | 124.019 | 1.00 | 28.69 | B | C |
| ATOM | 3224 | CA | PRO | 257 | 13.901 | −85.042 | 123.552 | 1.00 | 27.78 | B | C |
| ATOM | 3225 | CB | PRO | 257 | 14.551 | −85.894 | 124.647 | 1.00 | 28.06 | B | C |
| ATOM | 3226 | CG | PRO | 257 | 15.624 | −85.012 | 125.201 | 1.00 | 29.28 | B | C |
| ATOM | 3227 | C | PRO | 257 | 12.557 | −84.447 | 123.958 | 1.00 | 26.26 | B | C |
| ATOM | 3228 | O | PRO | 257 | 11.519 | −85.088 | 123.806 | 1.00 | 25.48 | B | O |
| ATOM | 3229 | N | GLU | 258 | 12.571 | −83.220 | 124.466 | 1.00 | 25.25 | B | N |
| ATOM | 3230 | CA | GLU | 258 | 11.330 | −82.562 | 124.851 | 1.00 | 24.25 | B | C |
| ATOM | 3231 | CB | GLU | 258 | 11.631 | −81.427 | 125.831 | 1.00 | 24.97 | B | C |
| ATOM | 3232 | CG | GLU | 258 | 12.345 | −81.953 | 127.069 | 1.00 | 25.66 | B | C |
| ATOM | 3233 | CD | GLU | 258 | 12.787 | −80.879 | 128.029 | 1.00 | 25.87 | B | C |
| ATOM | 3234 | OE1 | GLU | 258 | 13.412 | −79.897 | 127.581 | 1.00 | 25.45 | B | O |
| ATOM | 3235 | OE2 | GLU | 258 | 12.521 | −81.034 | 129.240 | 1.00 | 25.58 | B | O |
| ATOM | 3236 | C | GLU | 258 | 10.634 | −82.060 | 123.587 | 1.00 | 23.02 | B | C |
| ATOM | 3237 | O | GLU | 258 | 9.423 | −82.212 | 123.442 | 1.00 | 22.77 | B | O |
| ATOM | 3238 | N | TYR | 259 | 11.401 | −81.481 | 122.666 | 1.00 | 21.21 | B | N |
| ATOM | 3239 | CA | TYR | 259 | 10.846 | −81.004 | 121.399 | 1.00 | 20.28 | B | C |
| ATOM | 3240 | CB | TYR | 259 | 11.939 | −80.378 | 120.527 | 1.00 | 18.72 | B | C |
| ATOM | 3241 | CG | TYR | 259 | 12.113 | −78.882 | 120.686 | 1.00 | 18.66 | B | C |
| ATOM | 3242 | CD1 | TYR | 259 | 11.074 | −77.997 | 120.378 | 1.00 | 18.05 | B | C |
| ATOM | 3243 | CE1 | TYR | 259 | 11.251 | −76.616 | 120.487 | 1.00 | 17.04 | B | C |
| ATOM | 3244 | CD2 | TYR | 259 | 13.328 | −78.346 | 121.110 | 1.00 | 16.85 | B | C |
| ATOM | 3245 | CE2 | TYR | 259 | 13.515 | −76.976 | 121.220 | 1.00 | 15.76 | B | C |
| ATOM | 3246 | CZ | TYR | 259 | 12.479 | −76.114 | 120.905 | 1.00 | 17.18 | B | C |
| ATOM | 3247 | OH | TYR | 259 | 12.692 | −74.750 | 120.968 | 1.00 | 16.41 | B | O |
| ATOM | 3248 | C | TYR | 259 | 10.253 | −82.195 | 120.646 | 1.00 | 20.33 | B | C |
| ATOM | 3249 | O | TYR | 259 | 9.143 | −82.123 | 120.101 | 1.00 | 19.27 | B | O |
| ATOM | 3250 | N | VAL | 260 | 11.009 | −83.289 | 120.618 | 1.00 | 20.16 | B | N |
| ATOM | 3251 | CA | VAL | 260 | 10.588 | −84.498 | 119.930 | 1.00 | 22.13 | B | C |
| ATOM | 3252 | CB | VAL | 260 | 11.730 | −85.535 | 119.921 | 1.00 | 24.17 | B | C |
| ATOM | 3253 | CG1 | VAL | 260 | 11.205 | −86.882 | 119.500 | 1.00 | 24.73 | B | C |
| ATOM | 3254 | CG2 | VAL | 260 | 12.822 | −85.088 | 118.949 | 1.00 | 25.03 | B | C |
| ATOM | 3255 | C | VAL | 260 | 9.324 | −85.119 | 120.530 | 1.00 | 21.91 | B | C |
| ATOM | 3256 | O | VAL | 260 | 8.428 | −85.541 | 119.796 | 1.00 | 21.90 | B | O |
| ATOM | 3257 | N | LEU | 261 | 9.249 | −85.181 | 121.855 | 1.00 | 21.23 | B | N |
| ATOM | 3258 | CA | LEU | 261 | 8.074 | −85.746 | 122.516 | 1.00 | 22.69 | B | C |
| ATOM | 3259 | CB | LEU | 261 | 8.334 | −85.912 | 124.019 | 1.00 | 22.40 | B | C |
| ATOM | 3260 | CG | LEU | 261 | 9.193 | −87.127 | 124.378 | 1.00 | 22.56 | B | C |
| ATOM | 3261 | CD1 | LEU | 261 | 9.560 | −87.121 | 125.861 | 1.00 | 23.42 | B | C |
| ATOM | 3262 | CD2 | LEU | 261 | 8.419 | −88.390 | 124.019 | 1.00 | 21.06 | B | C |
| ATOM | 3263 | C | LEU | 261 | 6.842 | −84.869 | 122.283 | 1.00 | 22.97 | B | C |
| ATOM | 3264 | O | LEU | 261 | 5.721 | −85.365 | 122.182 | 1.00 | 23.28 | B | O |
| ATOM | 3265 | N | LEU | 262 | 7.063 | −83.565 | 122.191 | 1.00 | 22.71 | B | N |
| ATOM | 3266 | CA | LEU | 262 | 5.992 | −82.611 | 121.950 | 1.00 | 23.51 | B | C |
| ATOM | 3267 | CB | LEU | 262 | 6.568 | −81.194 | 122.027 | 1.00 | 24.71 | B | C |
| ATOM | 3268 | CG | LEU | 262 | 5.647 | −80.042 | 122.429 | 1.00 | 27.53 | B | C |
| ATOM | 3269 | CD1 | LEU | 262 | 5.105 | −80.278 | 123.839 | 1.00 | 27.55 | B | C |
| ATOM | 3270 | CD2 | LEU | 262 | 6.427 | −78.733 | 122.369 | 1.00 | 27.91 | B | C |
| ATOM | 3271 | C | LEU | 262 | 5.396 | −82.889 | 120.555 | 1.00 | 23.38 | B | C |
| ATOM | 3272 | O | LEU | 262 | 4.170 | −82.918 | 120.376 | 1.00 | 22.75 | B | O |
| ATOM | 3273 | N | ALA | 263 | 6.270 | −83.100 | 119.572 | 1.00 | 22.06 | B | N |
| ATOM | 3274 | CA | ALA | 263 | 5.834 | −83.399 | 118.215 | 1.00 | 22.18 | B | C |
| ATOM | 3275 | CB | ALA | 263 | 7.036 | −83.499 | 117.285 | 1.00 | 20.60 | B | C |
| ATOM | 3276 | C | ALA | 263 | 5.071 | −84.722 | 118.231 | 1.00 | 22.24 | B | C |
| ATOM | 3277 | O | ALA | 263 | 4.030 | −84.852 | 117.585 | 1.00 | 22.01 | B | O |
| ATOM | 3278 | N | ALA | 264 | 5.593 | −85.702 | 118.965 | 1.00 | 21.91 | B | N |
| ATOM | 3279 | CA | ALA | 264 | 4.938 | −87.005 | 119.073 | 1.00 | 22.99 | B | C |
| ATOM | 3280 | CB | ALA | 264 | 5.795 | −87.961 | 119.905 | 1.00 | 23.25 | B | C |
| ATOM | 3281 | C | ALA | 264 | 3.551 | −86.861 | 119.707 | 1.00 | 23.31 | B | C |
| ATOM | 3282 | O | ALA | 264 | 2.602 | −87.538 | 119.307 | 1.00 | 23.47 | B | O |
| ATOM | 3283 | N | MET | 265 | 3.434 | −85.987 | 120.702 | 1.00 | 23.39 | B | N |
| ATOM | 3284 | CA | MET | 265 | 2.152 | −85.774 | 121.359 | 1.00 | 24.89 | B | C |
| ATOM | 3285 | CB | MET | 265 | 2.326 | −84.923 | 122.617 | 1.00 | 26.27 | B | C |
| ATOM | 3286 | CG | MET | 265 | 2.960 | −85.692 | 123.761 | 1.00 | 28.18 | B | C |
| ATOM | 3287 | SD | MET | 265 | 3.160 | −84.705 | 125.239 | 1.00 | 30.04 | B | S |
| ATOM | 3288 | CE | MET | 265 | 1.493 | −84.692 | 125.842 | 1.00 | 28.25 | B | C |
| ATOM | 3289 | C | MET | 265 | 1.152 | −85.126 | 120.415 | 1.00 | 24.62 | B | C |
| ATOM | 3290 | O | MET | 265 | −0.040 | −85.407 | 120.487 | 1.00 | 24.02 | B | O |
| ATOM | 3291 | N | ALA | 266 | 1.638 | −84.258 | 119.531 | 1.00 | 24.92 | B | N |
| ATOM | 3292 | CA | ALA | 266 | 0.771 | −83.608 | 118.550 | 1.00 | 25.19 | B | C |
| ATOM | 3293 | CB | ALA | 266 | 1.531 | −82.476 | 117.835 | 1.00 | 23.17 | B | C |
| ATOM | 3294 | C | ALA | 266 | 0.325 | −84.668 | 117.532 | 1.00 | 25.75 | B | C |
| ATOM | 3295 | O | ALA | 266 | −0.847 | −84.734 | 117.156 | 1.00 | 25.62 | B | O |
| ATOM | 3296 | N | LEU | 267 | 1.278 | −85.491 | 117.092 | 1.00 | 25.83 | B | N |
| ATOM | 3297 | CA | LEU | 267 | 1.022 | −86.554 | 116.123 | 1.00 | 26.87 | B | C |
| ATOM | 3298 | CB | LEU | 267 | 2.296 | −87.377 | 115.891 | 1.00 | 26.43 | B | C |
| ATOM | 3299 | CG | LEU | 267 | 2.542 | −88.069 | 114.541 | 1.00 | 27.28 | B | C |
| ATOM | 3300 | CD1 | LEU | 267 | 3.607 | −89.134 | 114.721 | 1.00 | 26.00 | B | C |
| ATOM | 3301 | CD2 | LEU | 267 | 1.281 | −88.692 | 113.999 | 1.00 | 27.86 | B | C |
| ATOM | 3302 | C | LEU | 267 | −0.081 | −87.505 | 116.596 | 1.00 | 27.40 | B | C |
| ATOM | 3303 | O | LEU | 267 | −1.059 | −87.741 | 115.883 | 1.00 | 27.12 | B | O |
| ATOM | 3304 | N | PHE | 268 | 0.084 | −88.047 | 117.799 | 1.00 | 28.25 | B | N |
| ATOM | 3305 | CA | PHE | 268 | −0.876 | −89.006 | 118.341 | 1.00 | 30.10 | B | C |
| ATOM | 3306 | CB | PHE | 268 | −0.145 | −90.017 | 119.233 | 1.00 | 28.85 | B | C |
| ATOM | 3307 | CG | PHE | 268 | 0.824 | −90.889 | 118.483 | 1.00 | 28.85 | B | C |
| ATOM | 3308 | CD1 | PHE | 268 | 2.184 | −90.857 | 118.777 | 1.00 | 28.83 | B | C |
| ATOM | 3309 | CD2 | PHE | 268 | 0.380 | −91.709 | 117.451 | 1.00 | 28.35 | B | C |
| ATOM | 3310 | CE1 | PHE | 268 | 3.091 | −91.629 | 118.047 | 1.00 | 30.17 | B | C |
| ATOM | 3311 | CE2 | PHE | 268 | 1.276 | −92.486 | 116.713 | 1.00 | 29.06 | B | C |
| ATOM | 3312 | CZ | PHE | 268 | −2.635 | −92.447 | 117.008 | 1.00 | 28.98 | B | C |
| ATOM | 3313 | C | PHE | 268 | −2.078 | −88.427 | 119.084 | 1.00 | 31.65 | B | C |
| ATOM | 3314 | O | PHE | 268 | −2.299 | −88.727 | 120.255 | 1.00 | 31.36 | B | O |
| ATOM | 3315 | N | SER | 269 | −2.858 | −87.611 | 118.386 | 1.00 | 34.17 | B | N |
| ATOM | 3316 | CA | SER | 269 | −4.054 | −87.004 | 118.961 | 1.00 | 36.81 | B | C |
| ATOM | 3317 | CB | SER | 269 | −4.240 | −85.581 | 118.435 | 1.00 | 36.42 | B | C |
| ATOM | 3318 | OG | SER | 269 | −3.138 | −84.767 | 118.789 | 1.00 | 37.62 | B | O |
| ATOM | 3319 | C | SER | 269 | −5.260 | −87.854 | 118.566 | 1.00 | 38.85 | B | C |
| ATOM | 3320 | O | SER | 269 | −5.567 | −87.996 | 117.380 | 1.00 | 38.42 | B | O |
| ATOM | 3321 | N | PRO | 270 | −5.959 | −88.428 | 119.561 | 1.00 | 40.51 | B | N |
| ATOM | 3322 | CD | PRO | 270 | −5.665 | −88.310 | 121.002 | 1.00 | 40.82 | B | C |
| ATOM | 3323 | CA | PRO | 270 | −7.138 | −89.275 | 119.336 | 1.00 | 41.97 | B | C |
| ATOM | 3324 | CB | PRO | 270 | −7.342 | −89.938 | 120.695 | 1.00 | 41.86 | B | C |
| ATOM | 3325 | CG | PRO | 270 | −6.941 | −88.839 | 121.643 | 1.00 | 41.56 | B | C |
| ATOM | 3326 | C | PRO | 270 | −8.391 | −88.532 | 118.871 | 1.00 | 43.29 | B | C |
| ATOM | 3327 | O | PRO | 270 | −9.311 | −89.141 | 118.325 | 1.00 | 43.76 | B | O |
| ATOM | 3328 | N | ASP | 271 | −8.429 | −87.222 | 119.088 | 1.00 | 44.33 | B | N |
| ATOM | 3329 | CA | ASP | 271 | −9.585 | −86.424 | 118.696 | 1.00 | 45.68 | B | C |
| ATOM | 3330 | CB | ASP | 271 | −9.881 | −85.370 | 119.774 | 1.00 | 46.97 | B | C |
| ATOM | 3331 | CG | ASP | 271 | −8.764 | −84.350 | 119.928 | 1.00 | 48.52 | B | C |
| ATOM | 3332 | OD1 | ASP | 271 | −7.578 | −84.724 | 119.795 | 1.00 | 49.44 | B | O |
| ATOM | 3333 | OD2 | ASP | 271 | −9.075 | −83.168 | 120.202 | 1.00 | 49.05 | B | O |
| ATOM | 3334 | C | ASP | 271 | −9.389 | −85.765 | 117.334 | 1.00 | 45.97 | B | C |
| ATOM | 3335 | O | ASP | 271 | −9.848 | −84.651 | 117.089 | 1.00 | 45.91 | B | O |
| ATOM | 3336 | N | ARG | 272 | −8.707 | −86.476 | 116.444 | 1.00 | 45.79 | B | N |
| ATOM | 3337 | CA | ARG | 272 | −8.448 | −85.977 | 115.108 | 1.00 | 45.77 | B | C |
| ATOM | 3338 | CB | ARG | 272 | −7.094 | −86.489 | 114.612 | 1.00 | 44.26 | B | C |
| ATOM | 3339 | CG | ARG | 272 | −6.217 | −85.425 | 113.998 | 1.00 | 41.89 | B | C |
| ATOM | 3340 | CD | ARG | 272 | −5.168 | −84.917 | 114.976 | 1.00 | 39.65 | B | C |
| ATOM | 3341 | NE | ARG | 272 | −5.198 | −83.463 | 115.076 | 1.00 | 38.37 | B | N |
| ATOM | 3342 | CZ | ARG | 272 | −4.192 | −82.703 | 115.503 | 1.00 | 37.88 | B | C |
| ATOM | 3343 | NH1 | ARG | 272 | −3.037 | −83.239 | 115.876 | 1.00 | 37.41 | B | N |
| ATOM | 3344 | NH2 | ARG | 272 | −4.351 | −81.392 | 115.565 | 1.00 | 36.36 | B | N |
| ATOM | 3345 | C | ARG | 272 | −9.552 | −86.476 | 114.182 | 1.00 | 46.79 | B | C |
| ATOM | 3346 | O | ARG | 272 | −9.843 | −87.671 | 114.135 | 1.00 | 46.53 | B | O |
| ATOM | 3347 | N | PRO | 273 | −10.185 | −85.564 | 113.432 | 1.00 | 47.81 | B | N |
| ATOM | 3348 | CD | PRO | 273 | −9.883 | −84.133 | 113.255 | 1.00 | 48.15 | B | C |
| ATOM | 3349 | CA | PRO | 273 | −11.252 | −85.992 | 112.525 | 1.00 | 48.97 | B | C |
| ATOM | 3350 | CB | PRO | 273 | −11.603 | −84.704 | 111.772 | 1.00 | 48.79 | B | C |
| ATOM | 3351 | CG | PRO | 273 | −10.332 | −83.897 | 111.838 | 1.00 | 48.65 | B | C |
| ATOM | 3352 | C | PRO | 273 | −10.813 | −87.125 | 111.594 | 1.00 | 49.83 | B | C |
| ATOM | 3353 | O | PRO | 273 | −9.809 | −87.009 | 110.890 | 1.00 | 49.98 | B | O |
| ATOM | 3354 | N | GLY | 274 | −11.566 | −88.223 | 111.615 | 1.00 | 50.41 | B | N |
| ATOM | 3355 | CA | GLY | 274 | −11.257 | −89.363 | 110.769 | 1.00 | 51.30 | B | C |
| ATOM | 3356 | C | GLY | 274 | −10.578 | −90.544 | 111.443 | 1.00 | 52.28 | B | C |
| ATOM | 3357 | O | GLY | 274 | −10.367 | −91.577 | 110.806 | 1.00 | 52.21 | B | O |
| ATOM | 3358 | N | VAL | 275 | −10.234 | −90.412 | 112.722 | 1.00 | 53.24 | B | N |
| ATOM | 3359 | CA | VAL | 275 | −9.569 | −91.500 | 113.437 | 1.00 | 54.29 | B | C |
| ATOM | 3360 | CB | VAL | 275 | −8.796 | −90.984 | 114.678 | 1.00 | 54.20 | B | C |
| ATOM | 3361 | CG1 | VAL | 275 | −7.667 | −90.054 | 114.242 | 1.00 | 54.52 | B | C |
| ATOM | 3362 | CG2 | VAL | 275 | −9.742 | −90.268 | 115.621 | 1.00 | 54.74 | B | C |
| ATOM | 3363 | C | VAL | 275 | −10.525 | −92.604 | 113.886 | 1.00 | 54.76 | B | C |
| ATOM | 3364 | O | VAL | 275 | −11.548 | −92.348 | 114.524 | 1.00 | 54.98 | B | O |
| ATOM | 3365 | N | THR | 276 | −10.170 | −93.835 | 113.539 | 1.00 | 55.29 | B | N |
| ATOM | 3366 | CA | THR | 276 | −10.951 | −95.014 | 113.883 | 1.00 | 55.88 | B | C |
| ATOM | 3367 | CB | THR | 276 | −10.716 | −96.152 | 112.865 | 1.00 | 55.89 | B | C |
| ATOM | 3368 | OG1 | THR | 276 | −10.935 | −95.665 | 111.536 | 1.00 | 55.88 | B | O |
| ATOM | 3369 | CG2 | THR | 276 | −11.661 | −97.315 | 113.140 | 1.00 | 56.57 | B | C |
| ATOM | 3370 | C | THR | 276 | −10.538 | −95.527 | 115.260 | 1.00 | 56.10 | B | C |
| ATOM | 3371 | O | THR | 276 | −11.356 | −95.625 | 116.173 | 1.00 | 56.37 | B | O |
| ATOM | 3372 | N | GLN | 277 | −9.254 | −95.848 | 115.387 | 1.00 | 56.20 | B | N |
| ATOM | 3373 | CA | GLN | 277 | −8.673 | −96.374 | 116.618 | 1.00 | 56.61 | B | C |
| ATOM | 3374 | CB | GLN | 277 | −7.334 | −97.051 | 116.292 | 1.00 | 57.07 | B | C |
| ATOM | 3375 | CG | GLN | 277 | −7.407 | −98.557 | 116.026 | 1.00 | 58.02 | B | C |
| ATOM | 3376 | CD | GLN | 277 | −8.628 | −98.975 | 115.228 | 1.00 | 58.03 | B | C |
| ATOM | 3377 | OE1 | GLN | 277 | −8.868 | −98.484 | 114.126 | 1.00 | 59.02 | B | O |
| ATOM | 3378 | NE2 | GLN | 277 | −9.406 | −99.895 | 115.784 | 1.00 | 58.06 | B | N |
| ATOM | 3379 | C | GLN | 277 | −8.459 | −95.320 | 117.707 | 1.00 | 56.48 | B | C |
| ATOM | 3380 | O | GLN | 277 | −7.325 | −95.088 | 118.130 | 1.00 | 56.27 | B | O |
| ATOM | 3381 | N | ARG | 278 | −9.542 | −94.700 | 118.172 | 1.00 | 56.44 | B | N |
| ATOM | 3382 | CA | ARG | 278 | −9.443 | −93.672 | 119.205 | 1.00 | 56.32 | B | C |
| ATOM | 3383 | CB | ARG | 278 | −10.828 | −93.134 | 119.574 | 1.00 | 57.69 | B | C |
| ATOM | 3384 | CG | ARG | 278 | −11.466 | −92.272 | 118.495 | 1.00 | 59.91 | B | C |
| ATOM | 3385 | CD | ARG | 278 | −12.365 | −91.203 | 119.105 | 1.00 | 62.28 | B | C |
| ATOM | 3386 | NE | ARG | 278 | −11.597 | −90.261 | 119.921 | 1.00 | 64.42 | B | N |
| ATOM | 3387 | CZ | ARG | 278 | −12.106 | −89.193 | 120.534 | 1.00 | 65.26 | B | C |
| ATOM | 3388 | NH1 | ARG | 278 | −13.401 | −88.910 | 120.436 | 1.00 | 65.68 | B | N |
| ATOM | 3389 | NH2 | ARG | 278 | −11.312 | −88.400 | 121.243 | 1.00 | 65.69 | B | N |
| ATOM | 3390 | C | ARG | 278 | −8.729 | −94.136 | 120.467 | 1.00 | 55.50 | B | C |
| ATOM | 3391 | O | ARG | 278 | −7.716 | −93.559 | 120.850 | 1.00 | 55.10 | B | O |
| ATOM | 3392 | N | ASP | 279 | −9.252 | −95.172 | 121.116 | 1.00 | 54.66 | B | N |
| ATOM | 3393 | CA | ASP | 279 | −8.631 | −95.682 | 122.337 | 1.00 | 54.08 | B | C |
| ATOM | 3394 | CB | ASP | 279 | −9.395 | −96.901 | 122.869 | 1.00 | 55.13 | B | C |
| ATOM | 3395 | CG | ASP | 279 | −10.838 | −96.583 | 123.205 | 1.00 | 56.06 | B | C |
| ATOM | 3396 | OD1 | ASP | 279 | −11.079 | −95.570 | 123.898 | 1.00 | 56.83 | B | O |
| ATOM | 3397 | OD2 | ASP | 279 | −11.730 | −97.349 | 122.782 | 1.00 | 56.30 | B | O |
| ATOM | 3398 | C | ASP | 279 | −7.175 | −96.067 | 122.102 | 1.00 | 53.02 | B | C |
| ATOM | 3399 | O | ASP | 279 | −6.306 | −95.790 | 122.928 | 1.00 | 52.50 | B | O |
| ATOM | 3400 | N | GLU | 280 | −6.921 | −96.714 | 120.971 | 1.00 | 52.25 | B | N |
| ATOM | 3401 | CA | GLU | 280 | −5.580 | −97.151 | 120.607 | 1.00 | 51.45 | B | C |
| ATOM | 3402 | CB | GLU | 280 | −5.605 | −97.809 | 119.221 | 1.00 | 53.05 | B | C |
| ATOM | 3403 | CG | GLU | 280 | −6.329 | −99.165 | 119.136 | 1.00 | 55.94 | B | C |
| ATOM | 3404 | CD | GLU | 280 | −7.831 | −99.090 | 119.421 | 1.00 | 57.54 | B | C |
| ATOM | 3405 | OE1 | GLU | 280 | −8.530 | −98.264 | 118.794 | 1.00 | 57.75 | B | O |
| ATOM | 3406 | OE2 | GLU | 280 | −8.315 | −99.868 | 120.271 | 1.00 | 59.21 | B | O |
| ATOM | 3407 | C | GLU | 280 | −4.599 | −95.974 | 120.605 | 1.00 | 49.94 | B | C |
| ATOM | 3408 | O | GLU | 280 | −3.528 | −96.036 | 121.211 | 1.00 | 49.12 | B | O |
| ATOM | 3409 | N | ILE | 281 | −4.978 | −94.902 | 119.919 | 1.00 | 48.25 | B | N |
| ATOM | 3410 | CA | ILE | 281 | −4.146 | −93.712 | 119.827 | 1.00 | 46.84 | B | C |
| ATOM | 3411 | CB | ILE | 281 | −4.620 | −92.812 | 118.660 | 1.00 | 46.36 | B | C |
| ATOM | 3412 | CG2 | ILE | 281 | −3.836 | −91.504 | 118.645 | 1.00 | 45.98 | B | C |
| ATOM | 3413 | CG1 | ILE | 281 | −4.449 | −93.566 | 117.334 | 1.00 | 45.32 | B | C |
| ATOM | 3414 | CD1 | ILE | 281 | −4.913 | −92.803 | 116.116 | 1.00 | 45.69 | B | C |
| ATOM | 3415 | C | ILE | 281 | −4.133 | −92.912 | 121.132 | 1.00 | 46.37 | B | C |
| ATOM | 3416 | O | ILE | 281 | −3.146 | −92.248 | 121.445 | 1.00 | 46.02 | B | O |
| ATOM | 3417 | N | ASP | 282 | −5.223 | −92.986 | 121.894 | 1.00 | 46.17 | B | N |
| ATOM | 3418 | CA | ASP | 282 | −5.324 | −92.272 | 123.166 | 1.00 | 45.94 | B | C |
| ATOM | 3419 | CB | ASP | 282 | −6.719 | −92.451 | 123.777 | 1.00 | 46.56 | B | C |
| ATOM | 3420 | CG | ASP | 282 | −6.918 | −91.618 | 125.038 | 1.00 | 48.23 | B | C |
| ATOM | 3421 | OD1 | ASP | 282 | −6.723 | −90.381 | 124.981 | 1.00 | 48.42 | B | O |
| ATOM | 3422 | OD2 | ASP | 282 | −7.275 | −92.197 | 126.089 | 1.00 | 48.95 | B | O |
| ATOM | 3423 | C | ASP | 282 | −4.261 | −92.780 | 124.134 | 1.00 | 45.01 | B | C |
| ATOM | 3424 | O | ASP | 282 | −3.659 | −92.000 | 124.864 | 1.00 | 45.05 | B | O |
| ATOM | 3425 | N | GLN | 283 | −4.028 | −94.088 | 124.129 | 1.00 | 44.59 | B | N |
| ATOM | 3426 | CA | GLN | 283 | −3.018 | −94.696 | 124.994 | 1.00 | 43.67 | B | C |
| ATOM | 3427 | CB | GLN | 283 | −3.177 | −96.220 | 124.999 | 1.00 | 45.52 | B | C |
| ATOM | 3428 | CG | GLN | 283 | −4.438 | −96.701 | 125.705 | 1.00 | 48.81 | B | C |
| ATOM | 3429 | CD | GLN | 283 | −4.612 | −98.206 | 125.633 | 1.00 | 51.03 | B | C |
| ATOM | 3430 | OE1 | GLN | 283 | −3.712 | −98.967 | 126.000 | 1.00 | 52.12 | B | O |
| ATOM | 3431 | NE2 | GLN | 283 | −5.778 | −98.646 | 125.162 | 1.00 | 51.60 | B | N |
| ATOM | 3432 | C | GLN | 283 | −1.601 | −94.328 | 124.550 | 1.00 | 41.87 | B | C |
| ATOM | 3433 | O | GLN | 283 | −0.710 | −94.154 | 125.376 | 1.00 | 40.63 | B | O |
| ATOM | 3434 | N | LEU | 284 | −1.395 | −94.218 | 123.243 | 1.00 | 40.30 | B | N |
| ATOM | 3435 | CA | LEU | 284 | −0.085 | −93.856 | 122.718 | 1.00 | 39.29 | B | C |
| ATOM | 3436 | CB | LEU | 284 | −0.092 | −93.918 | 121.185 | 1.00 | 39.37 | B | C |
| ATOM | 3437 | CG | LEU | 284 | 0.335 | −95.249 | 120.550 | 1.00 | 39.93 | B | C |
| ATOM | 3438 | CD1 | LEU | 284 | −0.345 | −96.415 | 121.249 | 1.00 | 40.63 | B | C |
| ATOM | 3439 | CD2 | LEU | 284 | −0.001 | −95.239 | 119.067 | 1.00 | 38.88 | B | C |
| ATOM | 3440 | C | LEU | 284 | 0.303 | −92.456 | 123.188 | 1.00 | 37.95 | B | C |
| ATOM | 3441 | O | LEU | 284 | 1.425 | −92.231 | 123.638 | 1.00 | 37.80 | B | O |
| ATOM | 3442 | N | GLN | 285 | −0.625 | −91.514 | 123.092 | 1.00 | 36.96 | B | N |
| ATOM | 3443 | CA | GLN | 285 | −0.326 | −90.164 | 123.529 | 1.00 | 36.82 | B | C |
| ATOM | 3444 | CB | GLN | 285 | −1.483 | −89.215 | 123.239 | 1.00 | 37.92 | B | C |
| ATOM | 3445 | CG | GLN | 285 | −1.222 | −87.811 | 123.761 | 1.00 | 39.28 | B | C |
| ATOM | 3446 | CD | GLN | 285 | −2.277 | −86.841 | 123.334 | 1.00 | 40.09 | B | C |
| ATOM | 3447 | OE1 | GLN | 285 | −2.244 | −86.323 | 122.218 | 1.00 | 41.73 | B | O |
| ATOM | 3448 | NE2 | GLN | 285 | −3.241 | −86.594 | 124.211 | 1.00 | 40.92 | B | N |
| ATOM | 3449 | C | GLN | 285 | −0.013 | −90.141 | 125.016 | 1.00 | 36.21 | B | C |
| ATOM | 3450 | O | GLN | 285 | 0.902 | −89.438 | 125.436 | 1.00 | 34.84 | B | O |
| ATOM | 3451 | N | GLU | 286 | −0.768 | −90.904 | 125.810 | 1.00 | 35.91 | B | N |
| ATOM | 3452 | CA | GLU | 286 | −0.525 | −90.958 | 127.249 | 1.00 | 36.06 | B | C |
| ATOM | 3453 | CB | GLU | 286 | −1.528 | −91.884 | 127.937 | 1.00 | 38.35 | B | C |
| ATOM | 3454 | CG | GLU | 286 | −1.323 | −91.998 | 129.446 | 1.00 | 42.49 | B | C |
| ATOM | 3455 | CD | GLU | 286 | −1.209 | −90.639 | 130.124 | 1.00 | 44.67 | B | C |
| ATOM | 3456 | OE1 | GLU | 286 | −2.046 | −89.758 | 129.837 | 1.00 | 46.65 | B | O |
| ATOM | 3457 | OE2 | GLU | 286 | −0.288 | −90.451 | 130.950 | 1.00 | 46.57 | B | O |
| ATOM | 3458 | C | GLU | 286 | 0.895 | −91.449 | 127.499 | 1.00 | 35.04 | B | C |
| ATOM | 3459 | O | GLU | 286 | 1.584 | −90.955 | 128.387 | 1.00 | 34.20 | B | O |
| ATOM | 3460 | N | GLU | 287 | 1.328 | −92.426 | 126.708 | 1.00 | 34.29 | B | N |
| ATOM | 3461 | CA | GLU | 287 | 2.678 | −92.955 | 126.823 | 1.00 | 33.78 | B | C |
| ATOM | 3462 | CB | GLU | 287 | 2.869 | −94.123 | 125.851 | 1.00 | 35.48 | B | C |
| ATOM | 3463 | CG | GLU | 287 | 4.272 | −94.709 | 125.864 | 1.00 | 38.18 | B | C |
| ATOM | 3464 | CD | GLU | 287 | 4.415 | −95.922 | 124.964 | 1.00 | 40.29 | B | C |
| ATOM | 3465 | OE1 | GLU | 287 | 5.541 | −96.460 | 124.870 | 1.00 | 41.09 | B | O |
| ATOM | 3466 | OE2 | GLU | 287 | 3.405 | −96.341 | 124.354 | 1.00 | 41.61 | B | O |
| ATOM | 3467 | C | GLU | 287 | 3.671 | −91.835 | 126.506 | 1.00 | 31.95 | B | C |
| ATOM | 3468 | O | GLU | 287 | 4.702 | −91.711 | 127.157 | 1.00 | 31.06 | B | O |
| ATOM | 3469 | N | MET | 288 | 3.358 | −91.023 | 125.499 | 1.00 | 30.97 | B | N |
| ATOM | 3470 | CA | MET | 288 | 4.227 | −89.908 | 125.129 | 1.00 | 30.15 | B | C |
| ATOM | 3471 | CB | MET | 288 | 3.695 | −89.179 | 123.883 | 1.00 | 30.43 | B | C |
| ATOM | 3472 | CG | MET | 288 | 3.437 | −90.031 | 122.645 | 1.00 | 30.98 | B | C |
| ATOM | 3473 | SD | MET | 288 | 4.901 | −90.867 | 122.003 | 1.00 | 33.03 | B | S |
| ATOM | 3474 | CE | MET | 288 | 4.384 | −92.607 | 122.202 | 1.00 | 32.43 | B | C |
| ATOM | 3475 | C | MET | 288 | 4.251 | −88.919 | 126.300 | 1.00 | 28.89 | B | C |
| ATOM | 3476 | O | MET | 288 | 5.310 | −88.489 | 126.751 | 1.00 | 28.59 | B | O |
| ATOM | 3477 | N | ALA | 289 | 3.065 | −88.568 | 126.786 | 1.00 | 28.24 | B | N |
| ATOM | 3478 | CA | ALA | 289 | 2.920 | −87.622 | 127.887 | 1.00 | 28.40 | B | C |
| ATOM | 3479 | CB | ALA | 289 | 1.441 | −87.415 | 128.195 | 1.00 | 27.31 | B | C |
| ATOM | 3480 | C | ALA | 289 | 3.674 | −88.059 | 129.146 | 1.00 | 28.77 | B | C |
| ATOM | 3481 | O | ALA | 289 | 4.356 | −87.248 | 129.777 | 1.00 | 28.99 | B | O |
| ATOM | 3482 | N | LEU | 290 | 3.555 | −89.333 | 129.511 | 1.00 | 29.19 | B | N |
| ATOM | 3483 | CA | LEU | 290 | 4.248 | −89.850 | 130.688 | 1.00 | 29.74 | B | C |
| ATOM | 3484 | CB | LEU | 290 | 3.786 | −91.271 | 131.019 | 1.00 | 31.00 | B | C |
| ATOM | 3485 | CG | LEU | 290 | 2.366 | −91.417 | 131.574 | 1.00 | 33.11 | B | C |
| ATOM | 3486 | CD1 | LEU | 290 | 2.029 | −92.895 | 131.750 | 1.00 | 33.94 | B | C |
| ATOM | 3487 | CD2 | LEU | 290 | 2.259 | −90.677 | 132.908 | 1.00 | 34.21 | B | C |
| ATOM | 3488 | C | LEU | 290 | 5.750 | −89.850 | 130.475 | 1.00 | 29.25 | B | C |
| ATOM | 3489 | O | LEU | 290 | 6.510 | −89.582 | 131.400 | 1.00 | 29.29 | B | O |
| ATOM | 3490 | N | THR | 291 | 6.183 | −90.158 | 129.255 | 1.00 | 28.88 | B | N |
| ATOM | 3491 | CA | THR | 291 | 7.609 | −90.169 | 128.959 | 1.00 | 27.56 | B | C |
| ATOM | 3492 | CB | THR | 291 | 7.886 | −90.695 | 127.538 | 1.00 | 27.69 | B | C |
| ATOM | 3493 | OG1 | THR | 291 | 7.381 | −92.034 | 127.419 | 1.00 | 27.19 | B | O |
| ATOM | 3494 | CG2 | THR | 291 | 9.385 | −90.688 | 127.248 | 1.00 | 25.40 | B | C |
| ATOM | 3495 | C | THR | 291 | 8.159 | −88.753 | 129.090 | 1.00 | 27.38 | B | C |
| ATOM | 3496 | O | THR | 291 | 9.243 | −88.554 | 129.637 | 1.00 | 26.70 | B | O |
| ATOM | 3497 | N | LEU | 292 | 7.410 | −87.772 | 128.586 | 1.00 | 26.91 | B | N |
| ATOM | 3498 | CA | LEU | 292 | 7.829 | −86.378 | 128.681 | 1.00 | 27.05 | B | C |
| ATOM | 3499 | CB | LEU | 292 | 6.847 | −85.471 | 127.928 | 1.00 | 25.96 | B | C |
| ATOM | 3500 | CG | LEU | 292 | 7.139 | −83.963 | 127.939 | 1.00 | 25.61 | B | C |
| ATOM | 3501 | CD1 | LEU | 292 | 8.590 | −83.708 | 127.580 | 1.00 | 23.77 | B | C |
| ATOM | 3502 | CD2 | LEU | 292 | 6.202 | −83.244 | 126.966 | 1.00 | 24.54 | B | C |
| ATOM | 3503 | C | LEU | 292 | 7.916 | −85.960 | 130.151 | 1.00 | 27.73 | B | C |
| ATOM | 3504 | O | LEU | 292 | 8.875 | −85.308 | 130.554 | 1.00 | 26.74 | B | O |
| ATOM | 3505 | N | GLN | 293 | 6.917 | −86.338 | 130.949 | 1.00 | 29.30 | B | N |
| ATOM | 3506 | CA | GLN | 293 | 6.915 | −86.008 | 132.374 | 1.00 | 31.80 | B | C |
| ATOM | 3507 | CB | GLN | 293 | 5.663 | −86.564 | 133.059 | 1.00 | 33.01 | B | C |
| ATOM | 3508 | CG | GLN | 293 | 4.367 | −85.871 | 132.675 | 1.00 | 35.44 | B | C |
| ATOM | 3509 | CD | GLN | 293 | 3.151 | −86.491 | 133.354 | 1.00 | 37.29 | B | C |
| ATOM | 3510 | OE1 | GLN | 293 | 3.067 | −86.547 | 134.588 | 1.00 | 36.31 | B | O |
| ATOM | 3511 | NE2 | GLN | 293 | 2.201 | −86.964 | 132.546 | 1.00 | 38.33 | B | N |
| ATOM | 3512 | C | GLN | 293 | 8.155 | −86.594 | 133.051 | 1.00 | 32.77 | B | C |
| ATOM | 3513 | O | GLN | 293 | 8.908 | −85.875 | 133.707 | 1.00 | 32.11 | B | O |
| ATOM | 3514 | N | SER | 294 | 8.356 | −87.901 | 132.881 | 1.00 | 33.57 | B | N |
| ATOM | 3515 | CA | SER | 294 | 9.502 | −88.602 | 133.460 | 1.00 | 34.88 | B | C |
| ATOM | 3516 | CB | SER | 294 | 9.550 | −90.053 | 132.962 | 1.00 | 36.18 | B | C |
| ATOM | 3517 | OG | SER | 294 | 8.353 | −90.748 | 133.261 | 1.00 | 37.51 | B | O |
| ATOM | 3518 | C | SER | 294 | 10.808 | −87.913 | 133.090 | 1.00 | 34.78 | B | C |
| ATOM | 3519 | O | SER | 294 | 11.654 | −87.661 | 133.947 | 1.00 | 34.65 | B | O |
| ATOM | 3520 | N | TYR | 295 | 10.970 | −87.613 | 131.805 | 1.00 | 34.99 | B | N |
| ATOM | 3521 | CA | TYR | 295 | 12.175 | −86.954 | 131.331 | 1.00 | 35.00 | B | C |
| ATOM | 3522 | CB | TYR | 295 | 12.127 | −86.789 | 129.814 | 1.00 | 34.20 | B | C |
| ATOM | 3523 | CG | TYR | 295 | 13.357 | −86.115 | 129.263 | 1.00 | 33.37 | B | C |
| ATOM | 3524 | CD1 | TYR | 295 | 13.487 | −84.726 | 129.289 | 1.00 | 32.51 | B | C |
| ATOM | 3525 | CE1 | TYR | 295 | 14.627 | −84.106 | 128.811 | 1.00 | 32.85 | B | C |
| ATOM | 3526 | CD2 | TYR | 295 | 14.406 | −86.868 | 128.741 | 1.00 | 33.17 | B | C |
| ATOM | 3527 | CE2 | TYR | 295 | 15.550 | −86.259 | 128.260 | 1.00 | 32.70 | B | C |
| ATOM | 3528 | CZ | TYR | 295 | 15.654 | −84.879 | 128.295 | 1.00 | 33.54 | B | C |
| ATOM | 3529 | OH | TYR | 295 | 16.779 | −84.275 | 127.784 | 1.00 | 34.15 | B | O |
| ATOM | 3530 | C | TYR | 295 | 12.391 | −85.598 | 131.993 | 1.00 | 35.91 | B | C |
| ATOM | 3531 | O | TYR | 295 | 13.510 | −85.268 | 132.379 | 1.00 | 35.15 | B | O |
| ATOM | 3532 | N | ILE | 296 | 11.327 | −84.809 | 132.111 | 1.00 | 37.71 | B | N |
| ATOM | 3533 | CA | ILE | 296 | 11.418 | −83.492 | 132.741 | 1.00 | 40.23 | B | C |
| ATOM | 3534 | CB | ILE | 296 | 10.084 | −82.705 | 132.618 | 1.00 | 39.25 | B | C |
| ATOM | 3535 | CG2 | ILE | 296 | 10.153 | −81.424 | 133.447 | 1.00 | 38.18 | B | C |
| ATOM | 3536 | CG1 | ILE | 296 | 9.797 | −82.378 | 131.151 | 1.00 | 38.83 | B | C |
| ATOM | 3537 | CD1 | ILE | 296 | 8.486 | −81.651 | 130.932 | 1.00 | 38.48 | B | C |
| ATOM | 3538 | C | ILE | 296 | 11.751 | −83.639 | 134.227 | 1.00 | 42.98 | B | C |
| ATOM | 3539 | O | ILE | 296 | 12.617 | −82.934 | 134.752 | 1.00 | 42.72 | B | O |
| ATOM | 3540 | N | LYS | 297 | 11.050 | −84.552 | 134.897 | 1.00 | 46.09 | B | N |
| ATOM | 3541 | CA | LYS | 297 | 11.263 | −84.799 | 136.317 | 1.00 | 49.98 | B | C |
| ATOM | 3542 | CB | LYS | 297 | 10.432 | −85.997 | 136.786 | 1.00 | 49.95 | B | C |
| ATOM | 3543 | CG | LYS | 297 | 8.949 | −85.718 | 136.966 | 1.00 | 50.90 | B | C |
| ATOM | 3544 | CD | LYS | 297 | 8.231 | −86.957 | 137.487 | 1.00 | 51.71 | B | C |
| ATOM | 3545 | CE | LYS | 297 | 6.745 | −86.713 | 137.702 | 1.00 | 51.90 | B | C |
| ATOM | 3546 | NZ | LYS | 297 | 6.063 | −87.944 | 138.193 | 1.00 | 52.46 | B | N |
| ATOM | 3547 | C | LYS | 297 | 12.730 | −85.055 | 136.632 | 1.00 | 52.54 | B | C |
| ATOM | 3548 | O | LYS | 297 | 13.306 | −84.405 | 137.500 | 1.00 | 53.13 | B | O |
| ATOM | 3549 | N | GLY | 298 | 13.335 | −85.998 | 135.919 | 1.00 | 55.39 | B | N |
| ATOM | 3550 | CA | GLY | 298 | 14.728 | −86.318 | 136.167 | 1.00 | 59.24 | B | C |
| ATOM | 3551 | C | GLY | 298 | 15.742 | −85.421 | 135.485 | 1.00 | 62.03 | B | C |
| ATOM | 3552 | O | GLY | 298 | 16.926 | −85.452 | 135.824 | 1.00 | 62.62 | B | O |
| ATOM | 3553 | N | GLN | 299 | 15.293 | −84.610 | 134.536 | 1.00 | 64.81 | B | N |
| ATOM | 3554 | CA | GLN | 299 | 16.210 | −83.740 | 133.816 | 1.00 | 67.60 | B | C |
| ATOM | 3555 | CB | GLN | 299 | 15.488 | −83.006 | 132.688 | 1.00 | 67.76 | B | C |
| ATOM | 3556 | CG | GLN | 299 | 16.447 | −82.333 | 131.729 | 1.00 | 68.45 | B | C |
| ATOM | 3557 | CD | GLN | 299 | 17.668 | −83.194 | 131.453 | 1.00 | 68.63 | B | C |
| ATOM | 3558 | OE1 | GLN | 299 | 17.553 | −84.402 | 131.236 | 1.00 | 68.95 | B | O |
| ATOM | 3559 | NE2 | GLN | 299 | 18.845 | −82.576 | 131.457 | 1.00 | 68.63 | B | N |
| ATOM | 3560 | C | GLN | 299 | 16.927 | −82.731 | 134.697 | 1.00 | 69.34 | B | C |
| ATOM | 3561 | O | GLN | 299 | 16.312 | −82.047 | 135.518 | 1.00 | 69.59 | B | O |
| ATOM | 3562 | N | GLN | 300 | 18.240 | −82.655 | 134.495 | 1.00 | 71.45 | B | N |
| ATOM | 3563 | CA | GLN | 300 | 19.136 | −81.764 | 135.221 | 1.00 | 73.26 | B | C |
| ATOM | 3564 | CB | GLN | 300 | 20.562 | −81.917 | 134.676 | 1.00 | 73.69 | B | C |
| ATOM | 3565 | CG | GLN | 300 | 21.314 | −83.126 | 135.213 | 1.00 | 74.64 | B | C |
| ATOM | 3566 | CD | GLN | 300 | 20.675 | −84.451 | 134.846 | 1.00 | 75.14 | B | C |
| ATOM | 3567 | OE1 | GLN | 300 | 20.497 | −84.765 | 133.667 | 1.00 | 75.32 | B | O |
| ATOM | 3568 | NE2 | GLN | 300 | 20.331 | −85.241 | 135.858 | 1.00 | 75.53 | B | N |
| ATOM | 3569 | C | GLN | 300 | 18.720 | −80.292 | 135.204 | 1.00 | 74.21 | B | C |
| ATOM | 3570 | O | GLN | 300 | 17.533 | −79.984 | 135.303 | 1.00 | 74.60 | B | O |
| ATOM | 3571 | N | ARG | 301 | 19.703 | −79.397 | 135.073 | 1.00 | 75.01 | B | N |
| ATOM | 3572 | CA | ARG | 301 | 19.487 | −77.948 | 135.086 | 1.00 | 75.30 | B | C |
| ATOM | 3573 | CB | ARG | 301 | 18.678 | −77.484 | 133.858 | 1.00 | 75.26 | B | C |
| ATOM | 3574 | CG | ARG | 301 | 17.192 | −77.794 | 133.901 | 1.00 | 75.18 | B | C |
| ATOM | 3575 | CD | ARG | 301 | 16.505 | −77.521 | 132.580 | 1.00 | 75.06 | B | C |
| ATOM | 3576 | NE | ARG | 301 | 15.450 | −78.504 | 132.349 | 1.00 | 74.97 | B | N |
| ATOM | 3577 | CZ | ARG | 301 | 14.658 | −78.536 | 131.281 | 1.00 | 74.61 | B | C |
| ATOM | 3578 | NH1 | ARG | 301 | 14.779 | −77.630 | 130.317 | 1.00 | 74.17 | B | N |
| ATOM | 3579 | NH2 | ARG | 301 | 13.750 | −79.493 | 131.174 | 1.00 | 74.08 | B | N |
| ATOM | 3580 | C | ARG | 301 | 18.754 | −77.615 | 136.384 | 1.00 | 75.55 | B | C |
| ATOM | 3581 | O | ARG | 301 | 17.633 | −78.071 | 136.615 | 1.00 | 75.54 | B | O |
| ATOM | 3582 | N | ARG | 302 | 19.381 | −76.816 | 137.240 | 1.00 | 75.70 | B | N |
| ATOM | 3583 | CA | ARG | 302 | 18.746 | −76.492 | 138.507 | 1.00 | 75.72 | B | C |
| ATOM | 3584 | CB | ARG | 302 | 19.810 | −76.338 | 139.602 | 1.00 | 76.38 | B | C |
| ATOM | 3585 | CG | ARG | 302 | 19.234 | −76.395 | 141.018 | 1.00 | 77.18 | B | C |
| ATOM | 3586 | CD | ARG | 302 | 20.152 | −77.127 | 141.988 | 1.00 | 77.70 | B | C |
| ATOM | 3587 | NE | ARG | 302 | 21.423 | −76.437 | 142.207 | 1.00 | 78.27 | B | N |
| ATOM | 3588 | CZ | ARG | 302 | 21.562 | −75.290 | 142.868 | 1.00 | 78.26 | B | C |
| ATOM | 3589 | NH1 | ARG | 302 | 20.507 | −74.674 | 143.391 | 1.00 | 78.27 | B | N |
| ATOM | 3590 | NH2 | ARG | 302 | 22.767 | −74.760 | 143.016 | 1.00 | 78.37 | B | N |
| ATOM | 3591 | C | ARG | 302 | 17.762 | −75.317 | 138.574 | 1.00 | 75.23 | B | C |
| ATOM | 3592 | O | ARG | 302 | 17.499 | −74.803 | 139.663 | 1.00 | 75.35 | B | O |
| ATOM | 3593 | N | PRO | 303 | 17.202 | −74.860 | 137.430 | 1.00 | 74.61 | B | N |
| ATOM | 3594 | CD | PRO | 303 | 17.298 | −75.150 | 135.986 | 1.00 | 74.54 | B | C |
| ATOM | 3595 | CA | PRO | 303 | 16.273 | −73.750 | 137.667 | 1.00 | 73.58 | B | C |
| ATOM | 3596 | CB | PRO | 303 | 16.012 | −73.215 | 136.261 | 1.00 | 73.72 | B | C |
| ATOM | 3597 | CG | PRO | 303 | 16.073 | −74.451 | 135.427 | 1.00 | 74.08 | B | C |
| ATOM | 3598 | C | PRO | 303 | 15.010 | −74.319 | 138.321 | 1.00 | 72.47 | B | C |
| ATOM | 3599 | O | PRO | 303 | 14.156 | −73.579 | 138.807 | 1.00 | 72.47 | B | O |
| ATOM | 3600 | N | ARG | 304 | 14.920 | −75.648 | 138.330 | 1.00 | 71.25 | B | N |
| ATOM | 3601 | CA | ARG | 304 | 13.796 | −76.366 | 138.901 | 1.00 | 69.81 | B | C |
| ATOM | 3602 | CB | ARG | 304 | 13.947 | −76.484 | 140.423 | 1.00 | 71.29 | B | C |
| ATOM | 3603 | CG | ARG | 304 | 14.821 | −77.652 | 140.877 | 1.00 | 72.92 | B | C |
| ATOM | 3604 | CD | ARG | 304 | 14.673 | −77.891 | 142.376 | 1.00 | 74.32 | B | C |
| ATOM | 3605 | NE | ARG | 304 | 15.207 | −79.188 | 142.794 | 1.00 | 75.67 | B | N |
| ATOM | 3606 | CZ | ARG | 304 | 15.103 | −79.684 | 144.027 | 1.00 | 76.25 | B | C |
| ATOM | 3607 | NH1 | ARG | 304 | 14.484 | −78.995 | 144.980 | 1.00 | 76.56 | B | N |
| ATOM | 3608 | NH2 | ARG | 304 | 15.613 | −80.876 | 144.309 | 1.00 | 76.34 | B | N |
| ATOM | 3609 | C | ARG | 304 | 12.464 | −75.716 | 138.562 | 1.00 | 67.71 | B | C |
| ATOM | 3610 | O | ARG | 304 | 11.882 | −74.999 | 139.379 | 1.00 | 68.11 | B | O |
| ATOM | 3611 | N | ASP | 305 | 11.989 | −75.956 | 137.344 | 1.00 | 64.75 | B | N |
| ATOM | 3612 | CA | ASP | 305 | 10.708 | −75.417 | 136.929 | 1.00 | 61.23 | B | C |
| ATOM | 3613 | CB | ASP | 305 | 10.789 | −74.800 | 135.534 | 1.00 | 61.94 | B | C |
| ATOM | 3614 | CG | ASP | 305 | 9.459 | −74.232 | 135.082 | 1.00 | 62.41 | B | C |
| ATOM | 3615 | OD1 | ASP | 305 | 8.677 | −73.803 | 135.959 | 1.00 | 62.77 | B | O |
| ATOM | 3616 | OD2 | ASP | 305 | 9.202 | −74.194 | 133.860 | 1.00 | 62.51 | B | O |
| ATOM | 3617 | C | ASP | 305 | 9.679 | −76.536 | 136.956 | 1.00 | 58.50 | B | C |
| ATOM | 3618 | O | ASP | 305 | 9.625 | −77.382 | 136.059 | 1.00 | 58.07 | B | O |
| ATOM | 3619 | N | ARG | 306 | 8.878 | −76.543 | 138.016 | 1.00 | 54.93 | B | N |
| ATOM | 3620 | CA | ARG | 306 | 7.840 | −77.548 | 138.187 | 1.00 | 51.10 | B | C |
| ATOM | 3621 | CB | ARG | 306 | 7.408 | −77.604 | 139.651 | 1.00 | 53.42 | B | C |
| ATOM | 3622 | CG | ARG | 306 | 8.513 | −77.985 | 140.611 | 1.00 | 56.34 | B | C |
| ATOM | 3623 | CD | ARG | 306 | 8.016 | −77.954 | 142.041 | 1.00 | 59.35 | B | C |
| ATOM | 3624 | NE | ARG | 306 | 9.051 | −78.364 | 142.988 | 1.00 | 61.82 | B | N |
| ATOM | 3625 | CZ | ARG | 306 | 8.925 | −78.298 | 144.308 | 1.00 | 62.64 | B | C |
| ATOM | 3626 | NH1 | ARG | 306 | 9.923 | −78.686 | 145.090 | 1.00 | 62.90 | B | N |
| ATOM | 3627 | NH2 | ARG | 306 | 7.805 | −77.831 | 144.840 | 1.00 | 63.33 | B | N |
| ATOM | 3628 | C | ARG | 306 | 6.644 | −77.204 | 137.318 | 1.00 | 46.82 | B | C |
| ATOM | 3629 | O | ARG | 306 | 5.683 | −77.962 | 137.241 | 1.00 | 46.07 | B | O |
| ATOM | 3630 | N | PHE | 307 | 6.714 | −76.049 | 136.668 | 1.00 | 42.30 | B | N |
| ATOM | 3631 | CA | PHE | 307 | 5.647 | −75.583 | 135.807 | 1.00 | 38.22 | B | C |
| ATOM | 3632 | CB | PHE | 307 | 5.481 | −74.068 | 135.962 | 1.00 | 38.24 | B | C |
| ATOM | 3633 | CG | PHE | 307 | 5.179 | −73.628 | 137.369 | 1.00 | 38.97 | B | C |
| ATOM | 3634 | CD1 | PHE | 307 | 6.188 | −73.556 | 138.326 | 1.00 | 39.66 | B | C |
| ATOM | 3635 | CD2 | PHE | 307 | 3.883 | −73.296 | 137.741 | 1.00 | 39.64 | B | C |
| ATOM | 3636 | CE1 | PHE | 307 | 5.915 | −73.160 | 139.632 | 1.00 | 40.08 | B | C |
| ATOM | 3637 | CE2 | PHE | 307 | 3.595 | −72.898 | 139.047 | 1.00 | 40.42 | B | C |
| ATOM | 3638 | CZ | PHE | 307 | 4.616 | −72.830 | 139.993 | 1.00 | 40.42 | B | C |
| ATOM | 3639 | C | PHE | 307 | 5.901 | −75.924 | 134.337 | 1.00 | 35.22 | B | C |
| ATOM | 3640 | O | PHE | 307 | 5.030 | −75.731 | 133.494 | 1.00 | 34.63 | B | O |
| ATOM | 3641 | N | LEU | 308 | 7.085 | −76.445 | 134.036 | 1.00 | 32.23 | B | N |
| ATOM | 3642 | CA | LEU | 308 | 7.441 | −76.776 | 132.663 | 1.00 | 29.80 | B | C |
| ATOM | 3643 | CB | LEU | 308 | 8.818 | −77.445 | 132.617 | 1.00 | 30.13 | B | C |
| ATOM | 3644 | CG | LEU | 308 | 9.725 | −77.108 | 131.427 | 1.00 | 30.71 | B | C |
| ATOM | 3645 | CD1 | LEU | 308 | 10.806 | −78.172 | 131.314 | 1.00 | 31.09 | B | C |
| ATOM | 3646 | CD2 | LEU | 308 | 8.932 | −77.041 | 130.142 | 1.00 | 30.29 | B | C |
| ATOM | 3647 | C | LEU | 308 | 6.420 | −77.671 | 131.956 | 1.00 | 27.96 | B | C |
| ATOM | 3648 | O | LEU | 308 | 5.892 | −77.296 | 130.911 | 1.00 | 26.38 | B | O |
| ATOM | 3649 | N | TYR | 309 | 6.146 | −78.847 | 132.517 | 1.00 | 26.33 | B | N |
| ATOM | 3650 | CA | TYR | 309 | 5.199 | −79.770 | 131.896 | 1.00 | 26.08 | B | C |
| ATOM | 3651 | CB | TYR | 309 | 5.037 | −81.040 | 132.743 | 1.00 | 26.27 | B | C |
| ATOM | 3652 | CG | TYR | 309 | 4.121 | −82.074 | 132.109 | 1.00 | 26.57 | B | C |
| ATOM | 3653 | CD1 | TYR | 309 | 4.409 | −82.611 | 130.855 | 1.00 | 26.55 | B | C |
| ATOM | 3654 | CE1 | TYR | 309 | 3.559 | −83.533 | 130.249 | 1.00 | 27.76 | B | C |
| ATOM | 3655 | CD2 | TYR | 309 | 2.955 | −82.489 | 132.749 | 1.00 | 26.42 | B | C |
| ATOM | 3656 | CE2 | TYR | 309 | 2.092 | −83.414 | 132.152 | 1.00 | 28.03 | B | C |
| ATOM | 3657 | CZ | TYR | 309 | 2.404 | −83.930 | 130.902 | 1.00 | 27.91 | B | C |
| ATOM | 3658 | OH | TYR | 309 | 1.568 | −84.847 | 130.312 | 1.00 | 29.13 | B | O |
| ATOM | 3659 | C | TYR | 309 | 3.830 | −79.135 | 131.650 | 1.00 | 25.30 | B | C |
| ATOM | 3660 | O | TYR | 309 | 3.261 | −79.280 | 130.568 | 1.00 | 24.69 | B | O |
| ATOM | 3661 | N | ALA | 310 | 3.308 | −78.427 | 132.649 | 1.00 | 24.77 | B | N |
| ATOM | 3662 | CA | ALA | 310 | 2.007 | −77.780 | 132.519 | 1.00 | 23.64 | B | C |
| ATOM | 3663 | CB | ALA | 310 | 1.628 | −77.092 | 133.822 | 1.00 | 24.37 | B | C |
| ATOM | 3664 | C | ALA | 310 | 2.047 | −76.764 | 131.385 | 1.00 | 23.22 | B | C |
| ATOM | 3665 | O | ALA | 310 | 1.088 | −76.630 | 130.628 | 1.00 | 22.01 | B | O |
| ATOM | 3666 | N | LYS | 311 | 3.158 | −76.043 | 131.276 | 1.00 | 22.47 | B | N |
| ATOM | 3667 | CA | LYS | 311 | 3.315 | −75.052 | 130.217 | 1.00 | 22.76 | B | C |
| ATOM | 3668 | CB | LYS | 311 | 4.612 | −74.271 | 130.413 | 1.00 | 23.73 | B | C |
| ATOM | 3669 | CG | LYS | 311 | 4.563 | −73.270 | 131.550 | 1.00 | 25.88 | B | C |
| ATOM | 3670 | CD | LYS | 311 | 5.880 | −72.533 | 131.657 | 1.00 | 27.25 | B | C |
| ATOM | 3671 | CE | LYS | 311 | 5.884 | −71.568 | 132.820 | 1.00 | 29.50 | B | C |
| ATOM | 3672 | NZ | LYS | 311 | 7.240 | −70.971 | 133.000 | 1.00 | 31.45 | B | N |
| ATOM | 3673 | C | LYS | 311 | 3.309 | −75.710 | 128.838 | 1.00 | 20.98 | B | C |
| ATOM | 3674 | O | LYS | 311 | 2.728 | −75.185 | 127.900 | 1.00 | 21.17 | B | O |
| ATOM | 3675 | N | LEU | 312 | 3.949 | −76.865 | 128.721 | 1.00 | 20.58 | B | N |
| ATOM | 3676 | CA | LEU | 312 | 3.989 | −77.580 | 127.454 | 1.00 | 20.47 | B | C |
| ATOM | 3677 | CB | LEU | 312 | 4.968 | −78.753 | 127.550 | 1.00 | 20.31 | B | C |
| ATOM | 3678 | CG | LEU | 312 | 6.438 | −78.356 | 127.715 | 1.00 | 20.12 | B | C |
| ATOM | 3679 | CD1 | LEU | 312 | 7.304 | −79.599 | 127.750 | 1.00 | 20.29 | B | C |
| ATOM | 3680 | CD2 | LEU | 312 | 6.853 | −77.459 | 126.561 | 1.00 | 19.66 | B | C |
| ATOM | 3681 | C | LEU | 312 | 2.601 | −78.075 | 127.029 | 1.00 | 20.37 | B | C |
| ATOM | 3682 | O | LEU | 312 | 2.274 | −78.074 | 125.840 | 1.00 | 19.79 | B | O |
| ATOM | 3683 | N | LEU | 313 | 1.790 | −78.513 | 127.989 | 1.00 | 19.32 | B | N |
| ATOM | 3684 | CA | LEU | 313 | 0.444 | −78.967 | 127.659 | 1.00 | 19.16 | B | C |
| ATOM | 3685 | CB | LEU | 313 | −0.255 | −79.601 | 128.872 | 1.00 | 18.30 | B | C |
| ATOM | 3686 | CG | LEU | 313 | 0.303 | −80.942 | 129.369 | 1.00 | 19.24 | B | C |
| ATOM | 3687 | CD1 | LEU | 313 | −0.641 | −81.513 | 130.417 | 1.00 | 18.74 | B | C |
| ATOM | 3688 | CD2 | LEU | 313 | 0.456 | −81.929 | 128.205 | 1.00 | 19.01 | B | C |
| ATOM | 3689 | C | LEU | 313 | −0.345 | −77.762 | 127.173 | 1.00 | 18.59 | B | C |
| ATOM | 3690 | O | LEU | 313 | −1.131 | −77.866 | 126.235 | 1.00 | 18.15 | B | O |
| ATOM | 3691 | N | GLY | 314 | −0.123 | −76.617 | 127.810 | 1.00 | 18.61 | B | N |
| ATOM | 3692 | CA | GLY | 314 | −0.803 | −75.398 | 127.402 | 1.00 | 19.37 | B | C |
| ATOM | 3693 | C | GLY | 314 | −0.386 | −74.990 | 125.999 | 1.00 | 20.32 | B | C |
| ATOM | 3694 | O | GLY | 314 | −1.196 | −74.505 | 125.208 | 1.00 | 20.34 | B | O |
| ATOM | 3695 | N | LEU | 315 | 0.893 | −75.168 | 125.689 | 1.00 | 20.42 | B | N |
| ATOM | 3696 | CA | LEU | 315 | 1.394 | −74.829 | 124.367 | 1.00 | 20.97 | B | C |
| ATOM | 3697 | CB | LEU | 315 | 2.923 | −74.823 | 124.368 | 1.00 | 20.14 | B | C |
| ATOM | 3698 | CG | LEU | 315 | 3.491 | −73.558 | 125.027 | 1.00 | 21.61 | B | C |
| ATOM | 3699 | CD1 | LEU | 315 | 4.998 | −73.678 | 125.204 | 1.00 | 22.43 | B | C |
| ATOM | 3700 | CD2 | LEU | 315 | 3.141 | −72.341 | 124.174 | 1.00 | 20.16 | B | C |
| ATOM | 3701 | C | LEU | 315 | 0.847 | −75.791 | 123.318 | 1.00 | 20.93 | B | C |
| ATOM | 3702 | O | LEU | 315 | 0.560 | −75.380 | 122.200 | 1.00 | 21.86 | B | O |
| ATOM | 3703 | N | LEU | 316 | 0.693 | −77.064 | 123.670 | 1.00 | 20.78 | B | N |
| ATOM | 3704 | CA | LEU | 316 | 0.140 | −78.038 | 122.728 | 1.00 | 22.07 | B | C |

### REFERENCES

The references listed below as well as all references cited in the specification are incorporated herein by reference to the extent that they supplement, explain, provide a background for or teach methodology, techniques and/or compositions employed herein.
Adams P D, Pannu N S, Read R J & Brunger A T (1997) Cross-validated maximum likelihood enhances crystallographic simulated annealing refinement. Proc Natl Acad Sci USA 94: 5018-23.
Altschul S F, Gish W, Miller W, Myers E W & Lipman D J (1990) Basic Local Alignment Search Tool, J Mol Biol 215:403-410.
Agrawal S, ed., (1993) Protocols for Oligonucleotides and Analogs: Synthesis and Properties: Methods in Molecular Biology, volume 20, Humana Press, Totowa, N.J., United States of America.
Allen F H, Kennard O, Motherwell D S, Town W G, Watson D G (1973) The Cambridge Crystallographic Structural Data File. J. Chem. Doc. 13: 119.
Amemiya Y (1997) Methods in Enzymology, Vol. 276, pp. 233-243 Academic Press, San Diego, Calif., United States of America.
Appelt K (1993) Perspectives in Drug Discovery and Design 1:23-48.
Ausubel F M, Brent R, Kingston R E, Moore D D, Seidman J G, Smith J A & Struhl K, eds (1994) Current Protocols in Molecular Biology. Wiley, New York, United States of America.
Baes M, Gulick T, Choi H S, Martinoli M G, Simha D & Moore D D (1994) A New Orphan Member of the Nuclear Hormone Receptor Superfamily That Interacts with a Subset of Retinoic Acid Response Elements, Mol Cell Biol 14:1544-1551.
Bartlett et al. (1989) Special Pub., Royal Chem. Soc. 78: 182-196.
Beato M, Herrlich P & Schutz G (1995) Steroid Hormone Receptors: Many Actors in Search of a Plot, Cell 83:851-857.
Blundell & Johnson (1985) Meth Enzymol, 114A & 115B, Wyckoff et al., eds., Academic Press, San Diego, Calif., United States of America.
Blundell T, Carney D, Gardner S, Hayes F, Howlin B, Hubbard T, Overington J, Singh D A, Sibanda B L & Sutcliffe M (1988) 18th Sir Hans Krebs lecture. Knowledge-based protein modelling and design. Eur J Biochem 172:513-520.
Bohm, J Comput Aid Mol Des, 6: 61-78, 1992.
Bondi A, J Phys Chem, 68: 441451, 1964.
Boobbyer D N, Goodford P J, McWhinnie P M & Wade R C (1989) New hydrogen-bond potentials for use in determining energetically favorable binding sites on molecules of known structure. J Med Chem 32:1083-1094.
Bowie J U, Luthy R & Eisenberg D (1991) A method to identify protein sequences that fold into a known three-dimensional structure. Science 253: 164-170.
Brint A T & Willett P (1987) Pharmacophoric pattern matching in filesof 3D chemical structures: comparison of geometric searching algorithms. J Mol Graph 5:49-56.
Brooks et al., (1983) J Comp Chem 4:187-217.
Brünger A T (1992) X-PLOR, Version 3.1. A System for X-ray Crystallography and NMR, Yale University Press, New Haven, Conn., United States of America.
Brünger A T, Adams P D, Clore G M, DeLano W L, Gros P, Grosse-Kunstleve R W, Jiang J S, Kuszewski J, Nilges M, Pannu N S, Read R J, Rice L M, Simonson T & Warren G L (1998) Crystallography & Nmr System: A New Software Suite for Macromolecular Structure Determination, Acta Crystallogr D Biol Crystallogr 54:905-921.
Brzozowski et al. (1997) Nature 389:753.
Bugg C E, Carson W M & Montgomery J A (1993) Drugs by design. Scientific Am December:92-98.
Case et al., AMBER 5, University of California, San Francisco, 1997.
Cohen N C, Blaney J M, Humblet C, Gund P & Barry D C (1990) Molecular Modeling Software and Methods for Medicinal Chemistry. J Med Chem 33:883-894.
Collaborative Computational Project, Number 4, (1994) Acta Cryst. D50, 760-63.
Connolly M L (1983) Solvent-Accessible Surfaces of Proteins and Nucleic Acids. Science 221:709-713.
Cowtan, (1994), Joint CCP4 and ESF-EACBM Newsletter on Protein Crystallography, 31, p 34-8
Creighton (1983) Proteins: Structures and Molecular Principles, W.H. Freeman & Co., New York, United States of America.
Cwirla S E, Peters E A, Barrett R W & Dower W J (1990) Peptides on phage: a vast library of peptides for identifying ligands. Proc Natl Acad Sci USA, 87:6378-6382.
Dauter Z, Li M & Wlodawer A (2001) Practical experience with the use of halides for phasing macromolecular structures: a powerful tool for structural genomics. Acta Crystallog D Biol Crystallogr 57:239-49.
DesJarlais R L, Sheridan R P, Dixon J S, Kuntz I D & Venkataraghavan R (1986) Docking flexible ligands to macromolecular receptors by molecular shape. J. Med. Chem. 29:2149-2153.
DesJarlais R L, Sheridan R P, Seibel G L, Dixon J S, Kuntz I D & Venkataraghavan R (1988) Using shape complementarity as an initial screen in designing ligands for a receptor binding site of known three-dimensional structure. J. Med. Chem. 31:722-729.
Devlin J J, Panganiban L C & Devlin P E (1990) Random peptide libraries: a source of specific protein binding molecules. Science, 249:404-406.
Ducruix & Geige (1992) Crystallization of Nucleic Acids and Proteins: A Practical Approach, IRL Press, Oxford, England.
Dunbrack R L Jr., Gerloff D, Bower M, Chen X, Lichtarge O, & Cohen F E (1997). Meeting Review: The Second Meeting for the Critical Assessment of Protein Structure Prediction. Folding & Design 2:R27-R42.
Ebel S, Lane A N & Brown T (1992) Very stable mismatch duplexes: structural and thermodynamic studies on tandem G.A mismatches in DNA. Biochem. 31:12083-12086.
Eisen M B, Wiley D C, Karplus M & Hubbard R E (1994) Hook: A Program for Finding Novel Molecular Architectures That Satisfy the Chemical and Steric Requirements of a Macromolecule Binding Site. Proteins 19:199-221.
Erickson J W (1993) Perspectives in Drug Discovery and Design 1:109-128.
Fiser A, Do R K & Sali A (2000) Modeling of loops in protein structures. Protein Sci 9:1753-73.
Fitzgerald P M D (1988) MERLOT, an integrated package of computer programs for the determination of crystal structures by molecular replacement. J Appl Cryst 21:273-278.
Freedman L P (1999) Increasing the Complexity of Coactivation in Nuclear Receptor Signaling. Cell 97:5-8.
Goeddel D V (1990) Gene Expression Technology. Methods in Enzymology vol. 185, Academic Press, San Diego, Calif., United States of America.
Goodford P J (1985) A Computational Procedure for Determining Energetically Favorable Binding Sites on Biologically Important Macromolecules. J Med Chem 28:849-857.
Goodsell D S & Olson A J (1990) Automated Docking of Substrates to Proteins by Simulated Annealing. Proteins 8:195-202.
Goodwin B, Moore L B, Stoltz C M, McKee D D & Kliewer S A (2001) Regulation of the Human Cyp2b6 Gene by the Nuclear Pregnane X Receptor. Mol Pharmacol 60:427-431.
Greer J (1985) Model structure for the inflammatory protein C5a. Science 228:1055-1060.
Gribskov M & Burgess R R (1986) Sigma Factors from E. coli, B. Subtilis, Phage Sp01, and Phage T4 Are Homologous Proteins. Nucleic Acids Res 14:6745-6763.
Gronemeyer H & Laudet V (1995) Transcription Factors 3: Nuclear Receptors. Protein Profile 2:1173-1308.
Gutberlet T, Heinemann U & Steiner M (2001) Protein crystallography with neutrons—status and perspectives. Acta CrystallogrD Biol Crystallogr 57:349-54.
Handschin C, Podvinec M, Looser R, Amherd R & Meyer U A (2001) Multiple Enhancer Units Mediate Drug Induction of Cyp2h1 by Xenobiotic-Sensing Orphan Nuclear Receptor Chicken Xenobiotic Receptor. Mol Pharmacol 60:681-689.
Hauptman H (1997) Phasing Methods for Protein Crystallography. Curr Opin Struct Biol 7:672-680.
Hendrickson W A (2000) Synchrotron crystallography. Trends Biochem Sci 25:637-43.
Hendrickson W A & Ogata C M (1997) Methods in Enzymology, Vol. 276, pp. 494-523, San Diego, Calif., United States of America.
Honkakoski P, Zelko I, Sueyoshi T & Negishi M (1998) The Nuclear Orphan Receptor Car-Retinoid X Receptor Heterodimer Activates the Phenobarbital-Responsive Enhancer Module of the Cyp2b Gene. Mol Cell Biol 18:5652-5658.
Jakes et al. (1986) J Mol Graph 4:12-20.
Jakes et al. (1987) J Mol Graph 5:41-48.
Jancarik J & Kim S-H (1991) Sparse matrix sampling: a screening method for crystallisation of proteins. J Appl Cryst 24:409-411.
Jones T A, Zou J-Y, Cowan S & Kjeldgaard M (1991) Improved methods for building protein models into electron density maps and the locationof errors in these models. Acta Crystallogr A47:100-119.
Kahn R & Fourme R (1997) Methods in Enzymology, Vol. 276, pp. 268-286, Academic Press, San Diego, Calif., United States of America.
Kastner P, Mark M & Chambon P (1995) Nonsteroid Nuclear Receptors: What Are Genetic Studies Telling Us About Their Role in Real Life? Cell 83:859-869.
Kawamoto T, Sueyoshi T, Zelko I, Moore R, Washburn K & Negishi M (1999) Phenobarbital-Responsive Nuclear Translocation of the Receptor Car in Induction of the Cyp2b Gene. Mol Cell Biol 19:6318-6322.
Kleywegt G J & Brunger A T (1996) Checking your imagination: applications of the free R value. Structure 4:897-904.
Knighton D R, Pearson R B, Sowadski J M, Means A R, Ten Eyck L F, Taylor S S & Kemp B E (1992) Structural basis of the intrasteric regulation of myosin light chain kinases. Science 258:130-135.
Kuntz I D, Blaney J M, Oatley S J, Langridge R & Ferrin T E (1982) A Geometric Approach to Macromolecule-Ligand Interactions. J Mol Biol 161:269-288.
Kyte J & Doolittle R F (1982) A Simple Method for Displaying the Hydropathic Character of a Protein. J Mol Biol 157:105-132.
Lam P Y S, Jadhav P K, Eyermann C J, Hodge C N, Ru Y, Bacheler L T, Meek J L, Otto M J, Rayner M M, Wong Y N, Chang C-H, Weber P C, Jackson D A, Sharpe T R & Erickson-Viitanen S (1994) Rational Design of Potent, Bioavailable, Nonpeptide Cyclic Ureas as HIV Protease Inhibitors. Science 263:380-384.
Lambert, (1997) in Practical Application of Computer-Aided Drug Design, (Charifson, ed.), pp. 243-303, Marcel-Dekker, New York
Lanz R B & Rusconi S (1994) A Conserved Carboxy-Terminal Subdomain Is Important for Ligand Interpretation and Transactivation by Nuclear Receptors. Endocrinology 135:2183-2195.
Laskowski R A, McArthur M W, Moss D S & Thornton J M (1993) PROCHECK: A program to check the stereochemical quality of protein structures. J Appl Cryst 26:283-291.
Lattman E (1985) Diffraction Methods for Biological Macromolecules. Use of the Rotation and Translation Functions. Methods Enzymol 115:55-77.
Lattman E E (1996) No crystals no grant. Proteins 25:i-ii.
Lawrence M C & Davis P C (1992) CLIX: a search algorithm for finding novel ligands capable of binding proteins of known three-dimensional structure. Proteins 12:31-41.
Levitt D G (2001) A new software routine that automates the fitting of protein X-ray crystallographic electron-density maps. Acta Crystallogr D Biol Crystallogr 57:1013-1019.
Luthy R, Bowie J U & Eisenberg D (1992) Assessment of protein models with three-dimensional profiles. Nature 356:83-85.
Mangelsdorf D J & Evans R M (1995) The Rxr Heterodimers and Orphan Receptors. Cell 83:841-850.
Martin Y C (1992) 3d Database Searching in Drug Design. J Med Chem 35:2145-2154.
McCree D M et al. (1992) J. Mol. Graphics 10:44-46.
McKenna N J & O'Malley B W (2000) From Ligand to Response: Generating Diversity in Nuclear Receptor Coregulator Function. J Steroid Biochem Mol Biol 74:351-356.
McPherson et al. (1989) Preparation and Analysis of Protein Crystals, Robert E. Krieger Publishing Company, Malabar, Fla., United States of America.
McPherson, Eur J Biochem 189:1-23, 1990.
McRee, J Mol Graphics 10: 4447, 1992
McRee, Practical Protein Crystallography, Academic Press, New York, 1993.
Miller R, DeTitta G T, Jones R, Langs D A, Weeks C M & Hauptman H A (1993) On the Application of the Minimal Principle to Solve Unknown Structures. Science 259:1430.
Miranker A & Karplus M (1991) Functionality Maps of Binding Sites: A Multiple Copy Simultaneous Search Method. Proteins 11:29-34.
Moore L B, Parks D J, Jones S A, Bledsoe R K, Consler T G, Stimmel J B, Goodwin B, Liddle C, Blanchard S G, Willson T M, Collins J L & Kliewer S A (2000) Orphan Nuclear Receptors Constitutive Androstane Receptor and Pregnane X Receptor Share Xenobiotic and Steroid Ligands. J Biol Chem 275:15122-15127.
Moras D & Gronemeyer H (1998) The Nuclear Receptor Ligand-binding Domain: Structure and Function. Curr Opin Cell Biol 10:384-391.
Murshudov G N, Vagin M, Lebedev A, Wilson K S & Dodson E J (1999) Efficient anisotropic refinement of macromolecular structures using FFT. Acta Cryst D Biol Crystallogr 55:247-255.
Navaza, (1994) AMoRe: an automated package for molecular replacement, Acta. Cryst. A50: 157-163
Navia M A & Murcko M A (1992) Use of structural information in drug design Curr Opin Struct Biol 2:202-210.
Needleman S B & Wunsch C D (1970) A General Method Applicable to the Search for Similarities in the Amino Acid Sequence of Two Proteins. J Mol Biol 48:443-453.
Nicholls A, Sharp K A & Honig B (1991) Protein folding and association: insights from the interfacial and thermodynamic properties of hydrocarbons. Proteins 11:281-96.
Nishibata & ltai, (1991) Tetrahedron 47:8985-8990.
Nolte R T, Wisely G B, Westin S, Cobb J E, Lambert M H, Kurokawa R, Rosenfeld M G, Willson T M, Glass C K & Milburn M V (1998) Ligand-binding and Co-Activator Assembly of the Peroxisome Proliferator-Activated Receptor-Gamma. Nature 395:137-143.
Otwinowski (1993) in Proceedings of the CCP4 Study Weekend: Data Collection and Processing. Sawyer et al., eds., pp. 56-62, SERC Daresbury Laboratory, England.
Paech K, Webb P, Kuiper G G J M, Nilsson S, Gustafsson J-A, Kushner P J & Scanlan T S (1997) Differential Ligand Activation of Estrogen Receptors ERα and ER at Ap1 Sites. Science 277:1508-1510.
Parks D J, Blanchard S G, Bledsoe R K, Chandra G, Consler T G, Kliewer S A, Stimmel J B, Willson T M, Zavacki A M, Moore D D & Lehmann J M (1999) Bile Acids: Natural Ligands for an Orphan Nuclear Receptor. Science 284:1365-1368.
Pearlman et al., (1995) AMBER, a package of computer programs for applying molecular mechanics, a normal mode analysis, molecular dynamics and free energy calculations to simulate the structural and energetic properties of molecules Comput. Phys. Commun. 91: 1-41.
Perrakis A, Morris R & Lamzin V S (1999) Automated protein model building combined with iterative structure refinement. Nature Structural Biology 6:458-463.
Petsko G A (1985) Methods in Enzymology, Vol. 114, pp. 147-156, Academic Press, Orlando, Fla., United States of America.
Rarey et al., J Comput Aid Mol Des, 10:41-54, 1996.
Rossmann, ed, (1972) The Molecular Replacement Method, Gordon & Breach, New York, N.Y., United States of America.
Sambrook J & Russell D W (2001) Molecular Cloning: A Laboratory Manual, 3rd ed. Cold Spring Harbor Laboratory Press, Cold Spring Harbor, N.Y., United States of America.
Schulz G E & Schirmer R H (1979) Principles of Protein Structure, Springer-Verlag, New York, N.Y., United States of America.
Schwartz et al., eds., (1979), Atlas of Protein Sequence and Structure, National Biomedical Research Foundation, pp. 357-358.
Scott J K & Smith G P (1990) Searching for Peptide Ligands with an Epitope Library. Science 249:386-390.
Sheldrick, Acta Cryst, A46: 467, 1990.
Shiau et al., (1998) Cell 95:927.
Smith T F & Waterman M (1981) Comparison of Biosequences. Adv Appl Math 2:482489.
Stein B, Yang M X. Repression of the interleukin-6 promoter by estrogen receptor is mediated by NF-kB and C/EBP beta. Mol. Cell. Biol. 15, 4971-4979 (1995).
Stoddard B L (1998) New results using Laue diffraction and time-resolved crystallography. Curr Opin Struct Biol 8:612-8.
Tibanyenda N, De Bruin S H, Haasnoot C A, van der Marel G A, van Boom J H & Hilbers C W (1984) The effect of single base-pair mismatches on the duplex stability of d(T-A-T-T-A-A-T-A-T-C-A-A-G-T-T-G) . d(C-A-A-C-T-T-G-A-T-A-T-T-A-A-T-A). Eur J Biochem 139:19-27.
Tijssen P (1993) Overview of principles of hybridization and the strategy of nucleic acid probe assays in Laboratory Techniques in Biochemistry and Molecular Biology-Hybridization With Nucleic Acid Probes., Elsevier, New York, N.Y., United States of America.
Tora et al., (1988) Nature 333: 677-684.
Tosatto S C, Bindewald E, Hesser J & Manner R. (2002) A divide and conquer approach to fast loop modeling, Protein Eng 15:279-86.
Tsai and O'Malley, (1994) Molecular mechanisms of action of steroid/thyroid receptor superfamily members, Ann. Rev. Biochem. 63: 451-486
Tzukerman M T, Esty A, Santiso-Mere D, Danielian P, Parker M G, Stein R B, Pike J W & McDonnell D P (1994) Human Estrogen Receptor Transactivational Capacity Is Determined by Both Cellular and Promoter Context and Mediated by Two Functionally Distinct Intramolecular Regions. Mol Endocrinol 8:21-30.
Villieux F M D & Read R J (1997) Methods in Enzymology, Vol. 277, pp. 18-52, Academic Press, Orlando, Fla., United States of America.
Waxman D J (1999) P450 Gene Induction by Structurally Diverse Xenochemicals: Central Role of Nuclear Receptors Car, Pxr, and Ppar. Arch Biochem Biophys 369:11-23.
Weatherman R V, Fletterick R J & Scanlan T S (1999) Nuclear-Receptor Ligands and Ligand-binding Domains. Annu Rev Biochem 68:559-581.
Weber P C (1991) Physical Principles of Protein Crystallization. Adv Protein Chem 41:1-36.
Weeks C M, DeTitta G T, Hauptman H A, Thuman P & Miller R (1994) Structure solution by minimal-function phase refinement and Fourier filtering. II.

Implementation and applications. Acta Crystallogr A 50:210-20.
Wellner, (1971) Anal. Chem. 43:597.
West M L, Fairlie D P (1995) Targeting HIV-1 protease: a test of drug-design methodologies. Trends Pharmacol Sci 16:67-74.
Westbrook E M & Naday 1 (1997) Methods in Enzymology, Vol. 276, pp. 244-268, Academic Press, San Diego, Calif., United States of America.
Wlodawer A & Erickson J W (1993) Structure-based inhibitors of HIV-1 protease. Ann Rev Biochem 62:543-585.
Xiao, Li, Xiaoming, Cui, Madison, Vincent, White, Ronald E, and Cheng, K-C, (2002) Insights from a three-dimensional model into ligand-binding to Constitutive Active Receptor, Drug Metabolism and Disposition 30:951-956.
Xie W, Barwick J L, Simon C M, Pierce A M, Safe S, Blumberg B, Guzelian P S & Evans R M (2000a) Reciprocal Activation of Xenobiotic Response Genes by Nuclear Receptors Sxr/Pxr and Car. Genes Dev 14:3014-3023.
Xie W, Barwick J L, Downes M, Blumberg B, Simon C M, Nelson M C, Neuschwander-Tetri B A, Brunt E M, Guzelian P S & Evans R M (2000b) Humanized Xenobiotic Response in Mice Expressing Nuclear Receptor Sxr. Nature 406:435-439.
Zhang K Y J, Cowtan K & Main P (1997) Methods in Enzymology, Vol. 277, pp. 53-64, Academic Press, Orlando, Fla., United States of America.
U.S. Pat. No. 4,554,101
U.S. Pat. No. 6,008,033

It will be understood that various details of the invention can be changed without departing from the scope of the invention. Furthermore, the foregoing description is for the purpose of illustration only, and not for the purpose of limitation, the invention being defined by the claims.

What is claimed is:

1.-43. (canceled)

44. A method of screening a plurality of compounds for a ligand of a constitutive androstane receptor (CAR) ligand-binding domain polypeptide, the method comprising:
(a) providing a library of test samples;
(b) contacting a crystalline form comprising a constitutive androstane receptor (CAR) polypeptide in complex with a ligand with each test sample;
(c) detecting an interaction between a test sample and the crystalline constitutive androstane receptor (CAR) polypeptide in complex with a ligand;
(d) identifying a test sample that interacts with the crystalline constitutive androstane receptor (CAR) polypeptide in complex with a ligand; and
(e) isolating a test sample that interacts with the crystalline constitutive androstane receptor (CAR) polypeptide in complex with a ligand, whereby a plurality of compounds is screened for a ligand of a constitutive androstane receptor (CAR) ligand-binding domain polypeptide.

45. The method of claim 44, wherein the constitutive androstane receptor (CAR) polypeptide comprises a constitutive androstane receptor (CAR) ligand-binding domain.

46. The method of claim 44, wherein the constitutive androstane receptor (CAR) polypeptide is a human constitutive androstane receptor (CAR) polypeptide.

47. The method of claim 46, wherein the constitutive androstane receptor (CAR) polypeptide comprises the amino acid sequence of SEQ ID NO: 4.

48. The method of claim 44, wherein the library of test samples is bound to a substrate.

49. The method of claim 44, wherein the library of test samples is synthesized directly on a substrate.

50. The method of claim 44, wherein the ligand has a structure comprising Compound 1.

51.-120. (canceled)

121. The compound of Formula A 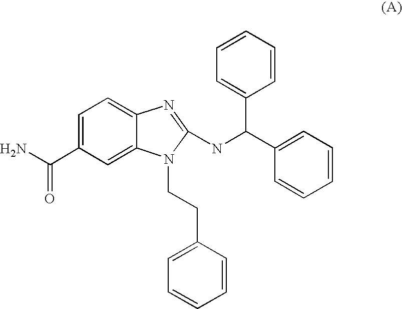
or a pharmaceutically acceptable salt thereof.
